(19) United States
(12) Patent Application Publication
(10) Pub. No.: US 2026/0151404 A1
(43) Pub. Date: Jun. 4, 2026

(54) BICYCLIC TETRAHYDROTHIAZEPINE DERIVATIVES

(71) Applicant: Hoffmann-La Roche Inc., Little Falls, NJ (US)

(72) Inventors: Marco BRANDSTAETTER, Basel (CH); Roman HUTTER, Allschwil (CH); Holger KUEHNE, Lörrach (DE); Thomas LUEBBERS, Hausen im Wiesental (DE); Nenad MANEVSKI, Riehen (CH); Laetitia Janine MARTIN, Saint-Louis (FR); Barbara Johanna MUELLER, Basel (CH)

(73) Assignee: Hoffmann-La Roche Inc., Little Falls, NJ (US)

(21) Appl. No.: 19/280,486

(22) Filed: Jul. 25, 2025

Related U.S. Application Data
(63) Continuation of application No. 19/101,062.

(30) Foreign Application Priority Data
Aug. 11, 2022 (EP) 22189820.8

(51) Int. Cl. A61K 31/554; A61P 35/00; C07D 417/04; C07D 417/14

(52) U.S. Cl. CPC A61K 31/554; A61P 35/00; C07D 417/04; C07D 417/14

(57) ABSTRACT

The present invention provides new bicyclic tetrahydrothiazepine derivatives having the general formula (I)

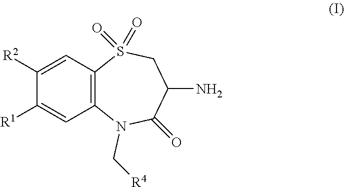

wherein R¹, R² and R⁴ are as defined herein, compositions including the compounds, processes of manufacturing the compounds and methods of using the compounds.

### FIELD OF THE INVENTION

The present invention relates to bicyclic tetrahydrothiazepine compounds which inhibit Diacylglycerol kinases (DGK) α and ζ and are useful as T-Cell activators, their manufacture and pharmaceutical compositions comprising said compounds.

The present compounds may be useful as immunotherapeutic agents for the treatment of human diseases. More specifically, the present compounds can be used alone or in combination with other immunotherapeutic agents in order to boost anti-cancer immunity.

### BACKGROUND OF THE INVENTION

Cancer immunity is a multistep process that is regulated by a series of negative immune checkpoint and positive co-stimulatory receptors and related intracellular signaling cascades that when effectively triggered can achieve antitumor response (Mellman, I., et al. (2011) Cancer Immunotherapy Comes of Age, Nature 480(7378), 480-489). Indeed, PD1/PDL1 targeting and other immune-checkpoint inhibitors have revolutionized cancer immunotherapy, but still more than 70% of patients do not benefit from immune-checkpoint inhibition.

Similarly, for T-cell bispecific antibodies, even in the most promising indication (Non-Hodgkin lymphoma), these T-cell binders (TCBs) achieve complete remissions in less than 50% of patients. T-cell exhaustion seems to play an important role in many of these examples of primary or secondary resistance to cancer immunotherapy. A possible reason for this lack of efficacy is that T-cell activation occurs via targeting and crosslinking of CD3 (signal 1), but co-stimulation e.g. via CD28 or 4-1BB (signal 2) is missing. This hypothesis was verified clinically for CAR T-cell therapy where it was shown that only after the incorporation of co-stimulatory domains, clinically relevant efficacy was observed.

Diacylglycerol kinases (DGKs) are lipid kinases that catalyze the conversion of Diacylglycerol (DAG) to phosphatidic acid (PA), thus limiting DAG-regulated and promoting PA-dependent functions (Merida, I., Avila-Flores, A., and Merino, E. 2008: Diacylglycerol kinases: at the hub of cell signalling. Biochem. J. 409 (1), 1-18). The DGK family consist of ten isoforms that can be grouped into five subtypes based on the presence of different regulatory domains within their structure. Beyond that, the lack of structural data as of now still hinders a more thorough understanding of the DGKs mode of action. Also information on certain prokaryotic DGK and other lipid kinases like sphingosine kinase and phosphatidylinositol-3-kinase (PI3K) has provided only limited insight into the DGK catalytic mechanisms which seems to be distinct from classical kinases (Arranz-Nicolás, J. and Mérida, I., 2020. Biological regulation of diacylglycerol kinases in normal and neoplastic tissues: New opportunities for cancer immunotherapy, Advances in Biological Regulation, Volume 75; Ma, Q., Gabelli, S. B., Raben, D. M., 2019: Diacylglycerol kinases: relationship to other lipid kinases. Adv Biol Regul 71, 104-110).

Although several isoforms within the DGK family have been described to play a role in cancer, the α and ζ isoforms are the ones that have been most deeply studied in this regard. As PA producers, both enzymes have been implicated in various processes promoting tumor growth and metastasis. On the other hand, as DAG consumers, DGKα and ζ have been extensively characterized as negative regulators of T cell responses (Riese, M. J., Moon, E. K., Johnson, B. D., Albelda, S. M., 2016. Diacylglycerol kinases (DGKs): novel targets for improving T cell activity in cancer. Front Cell Dev Biol 4,108; Noessner, E., 2017. DGK-alpha: a checkpoint in cancer-mediated immuno-inhibition and target for immunotherapy. Front Cell Dev Biol 5, 16; Sakane, F., Mizuno, S., Komenoi, S., 2016. Diacylglycerol kinases as emerging potential drug targets for a variety of diseases: an update. Front Cell Dev Biol 4, 82; Arranz-Nicolás, J. and Mérida, I., 2020. Biological regulation of diacylglycerol kinases in normal and neoplastic tissues: New opportunities for cancer immunotherapy, Advances in Biological Regulation, Volume 75).

These two isozymes DGKα and DGKζ are active downstream of CD28 and other costimulatory receptors as well as the T cell receptor (TCR), and their function is to limit the amount of DAG generated—and ultimately T-cell activation (Merida, I., Andrada, E., Gharbi, S. I., Avila-Flores, A., 2015. Redundant and specialized roles for diacylglycerol kinases alpha and zeta in the control of T cell functions. Sci. Signal. 8 (374); Shulga, Y. V., Topham, M. K., Epand, R. M., 2011. Regulation and functions of diacylglycerol kinases. Chem. Rev. 111 (10), 6186-6208.) A summary of representative DGK-regulated signaling pathways is shown in FIG. 1 (Sim, J. A.; Kim, J.; Yang, D. Beyond Lipid Signaling: Pleiotropic Effects of Diacylglycerol Kinases in Cellular Signaling. Int. J. Mol. Sci. 2020, 21, 6861): Activated PLC1 cleaves PIP2 in the plasma membrane to generate two secondary messengers, DAG and IP3. DAG activates PKC, Ras/MEK/ERK/AP-1 and NF-kB, while IP3 is involved in the activation of intracellular Ca2+ flux. The upregulated Ca2+ signaling in turn activates the transcription factor NFAT. In short, DAG production and levels determine the duration and intensity of the Ras/MEK/ERK and PKC-dependent signaling pathways, and they are central to T-cell activation. Thus, DGKs serve as intracellular checkpoints and inhibition of DGKs is expected to enhance T cell signaling pathways and T cell activation.

Experimental evidence suggests that enhanced DGK function and/or expression in tumor infiltrating T-cells (TILs) limits tumor destruction. Experiments with CAR T cells directed against human mesothelioma engrafted into nude mice demonstrated that tumor-infiltrating CAR T cells express elevated concentrations of surface inhibitory receptors, as well as the inhibitory enzymes SHIP-1, DGKα and DGKζ (Moon et al., 2014). Further, high DGKα expression was also observed in TIL isolated from human renal tumors (Prinz et al., 2012). In mouse mesoCAR T cells, dual deletion of DGKα and DGKζ results in enhanced cytokine expression and cytotoxicity against tumor cells (Riese et al., 2013). Similar results have been reported for human CAR T cells in which both DGKα and DGKζ expression were silenced using CRISPR/Cas9 (Jung et al., 2018). All these studies support a rationale for targeting DGKα/ζ in the development of anti-cancer therapies (Arranz-Nicolás, J. and Mérida, I., 2020. Biological regulation of diacylglycerol kinases in normal and neoplastic tissues: New opportunities for cancer immunotherapy, Advances in Biological Regulation, Volume 75; Riese, M. J., Moon, E. K., Johnson, B. D., Albelda, S. M., 2016. Diacylglycerol kinases (DGKs): novel targets for improving T cell activity in cancer. Front Cell Dev Biol 4, 108). Knock out mouse models provide further evidence: Mice lacking either DGKα or DGKζ showed a hyper-responsive T cell phenotype and improved anti-tumor immune activity (Riese, M. J., Grewal, J., Das, J., Zou, T., Patil, V., Chakraborty, A. K., Koretzky, G. A., 2011. Decreased diacylglycerol metabolism enhances ERK activation and augments CD8+ T cell functional responses. J. Biol. Chem. 286 (7), 5254-5265; Zha, Y., Marks, R., Ho, A. W., Peterson, A. C., Janardhan, S., Brown, I., Praveen, K., Stang, S., Stone, J. C., Gajewski, T. F., 2006. T cell anergy is reversed by active Ras and is regulated by diacylglycerol kinase-alpha. Nat. Immunol. 7 (11), 1166-1173; Olenchock, B. A., Guo, R., Carpenter, J. H., Jordan, M., Topham, M. K., Koretzky, G. A., Zhong, X. P., 2006a. Disruption of diacylglycerol metabolism impairs the induction of T cell anergy. Nat. Immunol. 7 (11), 1174-1181.)

Taken together, there is substantial evidence that DGKα and DGKζ are high value targets for cancer immunotherapy. At the same time, there is a lack of compounds with the ability to potently inhibit both DGKα and DGKζ with good selectivity over other diacylglycerol kinases, protein kinases, and/or other lipid kinases.

This invention describes such dual DGK α/ζ inhibitors with excellent selectivity over other protein kinases, across safety/off-target panels and vs. other lipid kinases. These compounds potently activate suboptimally stimulated T-cells and thereby act as intracellular enhancers of co-stimulatory signaling cascades. These DGK α/ζ inhibitors have the potential to increase proliferation, cytotoxicity and the life span of targeted T-cells which may result in improved anticancer activity of CPIs, CD3 engaging T-cell bispecifics and CAR T-cells.

Further, by engaging a signaling node central to both TCR and co-stimulatory receptors, it is plausible that these molecules enhance both signals 1 and 2 and thus single agent activity can be achieved, e.g. in inflamed tumors.

There is an ongoing need for new compounds capable of activating and proliferating T-cells, thus enabling the treatment, prevention and/or delay of progression of cancer.

It is, therefore, an object of this invention to provide compounds useful as DGKα/(inhibitors for the treatment or prevention or amelioration of such diseases with improved therapeutic properties, in particular improved pharmacokinetic properties.

### SUMMARY OF THE INVENTION

A first object of the present invention is a compound of formula (I)

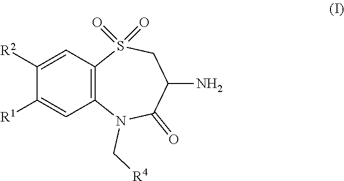

or a pharmaceutically acceptable salt thereof, wherein:
R¹ is oxadiazole, wherein R¹ is optionally substituted with one or more R¹⁰ which can be the same or different;
R² is selected from hydrogen and halogen;
R⁴ is selected from C₅₋₁₄-aryl and 5-14 membered heteroaryl, wherein R⁴ is optionally substituted with one or more R¹¹ which can be the same or different;
R¹⁰ is selected from:
   i) C₁₋₁₀-alkyl, optionally substituted with one or more halogen, amino, hydroxy, C₁₋₆-alkoxy, 3-10 membered cycloalkyl, phenyl, cyano;
   ii) C₃₋₁₀-cycloalkyl, optionally substituted with one or more halogen, cyano, amino;
   iii) 3-10 membered heterocyclyl, optionally substituted with one or more halogen, C₁₋₁₀-alkyl, amino, halo-C₁₋₆-alkyl, hydroxy, cyano, —C(O)O—(R^{10q}), C₃₋₁₀-cycloalkyl, wherein C₁₋₁₀-alkyl is optionally substituted with one or more hydroxy, C₁₋₆-alkoxy;
   iv) —N(R^{10e}R^{10f});
   v) heteroaryl, optionally substituted with one or more C₁₋₁₀-alkyl, halogen;
R^{10e} and R^{10f} are each independently selected from:
   i) hydrogen;
   ii) C₁₋₆-alkyl, optionally substituted with one or more, cyano, halogen, hydroxy;
   iii) C₃₋₁₀-cycloalkyl, optionally substituted with one or more halogen, C₁₋₁₀-alkyl;
R^{10q} is C₁₋₅-alkyl, wherein C₁₋₅-alkyl is optionally substituted with one or more hydroxy;
R¹¹ is selected from:
i) 5-6 membered heteroaryl, optionally substituted with one or more C₁₋₆-alkyl, C₃₋₁₀ cycloalkyl, halo-C₁₋₆-alkyl, C₁₋₆-alkoxy, halo-C₁₋₆-alkoxy, wherein C₃₋₁₀ cycloalkyl is optionally substituted with one or more halogen;
ii) phenyl, optionally substituted with one or more C₁₋₆-alkoxy, —OH, halo-C₁₋₆-alkyl.

A second object of the present invention is a process for the preparation of a compound of formula (I) as described above, or a pharmaceutically acceptable salt thereof, comprising reacting a compound of formula (IX)

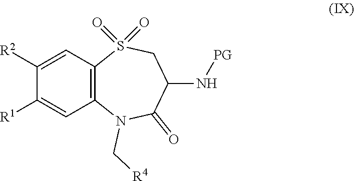

wherein R¹, R² and R⁴ are as defined herein and PG is an amino protecting group, with a suitable deprotection agent to form said compound of formula (I).

A third object of the present invention is a pharmaceutical composition comprising a compound of formula (I) as described above, or a pharmaceutically acceptable salt thereof, and a pharmaceutically acceptable excipient.

A forth object of the present invention is a compound of formula (I) as described herein, or a pharmaceutically acceptable salt thereof, for use in the treatment, prevention and/or delay of progression of cancer.

Unless otherwise defined, all technical and scientific terms used herein have the same meaning as commonly understood by one of ordinary skill in the art to which this invention belongs.

Although methods and materials similar or equivalent to those described herein can be used in the practice or testing of the invention, suitable methods and materials are described below.

All publications, patent applications, patents, and other references mentioned herein are incorporated by reference in their entirety.

The nomenclature used in this application is based on IUPAC systematic nomenclature, unless indicated otherwise.

### DETAILED DESCRIPTION OF THE INVENTION

### Definitions

“Alkoxy” refers to an alkyl group, as previously defined, attached to the parent molecular moiety via an oxygen atom. Unless otherwise specified, the alkoxy group contains 1 to 12 carbon atoms (“C₁₋₁₂-alkoxy”), preferably 1 to 10 carbon atoms (“C₁₋₁₀-alkoxy”), more preferably 1 to 6 carbon atoms (“C₁₋₆-alkoxy”). In some preferred embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy.

“Alkoxyalkyl” refers toan alkyl group, wherein at least one of the hydrogen atoms of the alkyl group has been replaced by an alkoxy group. Preferably, “alkoxyalkyl” refers to an alkyl group wherein 1, 2 or 3 hydrogen atoms, most preferably one hydrogen atom of the alkyl group have been replaced by an alkoxy group. Particularly preferred, yet non-limiting examples of alkoxyalkyl is methoxymethyl and 2-methoxyethyl.

“Alkyl” refers to a saturated linear (i.e. unbranched) or branched univalent hydrocarbon chain or combination thereof, having the number of carbon atoms designated (i.e., C₁₋₁₀ means one to ten carbon atoms). Particular alkyl groups are those having 1 to 20 carbon atoms (a “C₁₋₂₀ alkyl”), having 1 to 12 carbon atoms (a “C₁₋₁₂ alkyl”), having 1 to 10 carbon atoms (a “C₁₋₁₀ alkyl”), having 1 to 8 carbon atoms (a “C₁₋₈ alkyl”), having 1 to 6 carbon atoms (a “C₁₋₆ alkyl”), having 2 to 6 carbon atoms (a “C₂₋₆ alkyl”), or having 1 to 4 carbon atoms (a “C₁₋₄ alkyl”). Examples of alkyl group include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkynyl” refers to an unsaturated linear (i.e. unbranched) or branched univalent hydrocarbon chain or combination thereof, having at least one site of acetylenic unsaturation (i.e., having at least one moiety of the formula C≡C) having the number of carbon atoms designated (i.e. C₂₋₁₀ means two to ten carbon atoms). Particular alkynyl groups are those having 2 to 20 carbon atoms (a “C₂₋₂₀ alkynyl”), having 2 to 8 carbon atoms (a “C₂₋₈ alkynyl”), having 2 to 6 carbon atoms (a “C₂₋₆ alkynyl”), having 2 to 4 carbon atoms (a “C₂₄ alkynyl”). Examples of alkynyl group include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Amino”, alone or in combination with other groups, refers to NH₂.

“Aminoalkyl” refers to an alkyl group wherein one or more of the hydrogen atoms of the alkyl group have been replaced by an amino moiety.

“Aromatic” denotes the conventional idea of aromaticity as defined in the literature, in particular in IUPAC—Compendium of Chemical Terminology, 2^{nd} Edition, A. D. McNaught & A. Wilkinson (Eds). Blackwell Scientific Publications, Oxford (1997).

“Aryl” refers to a cyclic aromatic hydrocarbon moiety having a mono-, bi- or tricyclic aromatic ring of 5 to 14 carbon ring atoms (“C₅₋₁₄-aryl”). Bicyclic aryl ring systems include fused bicyclics having two fused five-membered aryl rings (denoted as 5-5), having a five-membered aryl ring and a fused six-membered aryl ring (denoted as 5-6 and as 6-5), and having two fused six-membered aryl rings (denoted as 6-6). The aryl group can be optionally substituted as defined herein. Examples of aryl substituent include, but are not limited to, phenyl, naphthyl, phenanthryl, fluorenyl, indenyl, pentalenyl, azulenyl, and the like. The term “aryl” also includes partially hydrogenated derivatives of the cyclic aromatic hydrocarbon moiety provided that at least one ring of the cyclic aromatic hydrocarbon moiety is aromatic, each being optionally substituted.

“Cancer” refers to a disease characterized by the presence of a neoplasm or tumor resulting from abnormal uncontrolled growth of cells (such cells being “cancer cells”). As used herein, the term cancer explicitly includes, but is not limited to, hepatocellular cancer, malignancies and hyperproliferative disorders of the colon (colon cancer), lung cancer, breast cancer, prostate cancer, melanoma, and ovarian cancer.

“Cyano”, alone or in combination with other groups, refers to CN (i.e. nitrile).

“Cyanoalkyl” refers to an alkyl group wherein one or more of the hydrogen atoms of the alkyl group have been replaced by a cyano moiety.

“Cycloalkyl” refers to a saturated or partially unsaturated carbocyclic moiety having mono-, bi- (including bridged bicyclic and cycloalkyl spiro substituent) or tricyclic rings and 3 to 10 carbon atoms i.e., (C₃-C₁₀)cycloalkyl) in the ring. The cycloalkyl moiety can optionally be substituted with one or more substituents. In particular aspects cycloalkyl contains from 3 to 8 carbon atoms (i.e., (C₃-C₈)cycloalkyl). In other particular aspects cycloalkyl contains from 3 to 6 carbon atoms (i.e., (C₃-C₆)cycloalkyl). Examples of cycloalkyl substituent include, but are not limited to, cyclopropyl, cyclobutyl, cyclopentyl, cyclohexyl, cycloheptyl, and partially unsaturated (cycloalkenyl) derivatives thereof (e.g. cyclopentenyl, cyclohexenyl, and cycloheptenyl), bicyclo[3.1.0]hexanyl, bicyclo[3.1.0]hexenyl, bicyclo[3.1.1]heptanyl, bicyclo[3.1.1]heptenyl and bicyclo[1.1.1]pentane. The cycloalkyl moiety can be attached in a “spiro-cycloalkyl” or “cycloalkyl spiro” fashion such as “spirocycloprpyl”

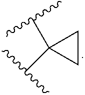 [SMILES: *C1(*)CC1]

“ECₓ” refers to the effective concentration e.g. in medium or in the plasma of a particular compound required for obtaining x % of the maximum of a particular effect in vitro or in vivo. Examples of “ECₓ” are EC₂₀, EC₅₀ and EC₁₀₀ denoting the concentration of a particular compound in medium or plasma required for obtaining 20%, 50% and 100%, respectively, of the maximum of a particular effect in vitro or in vivo. “Haloalkoxy” refers to an alkoxy group in which at least one Halogen takes the place of each H in the hydrocarbon making up the alkyl moiety of the alkoxy group. An example of a haloalkoxy group is difluoromethoxy (—OCHF₂), trifluoromethoxy (—OCF₃).

“Haloaryl” refers to an aryl wherein at least one hydrogen has been substituted with an halogen.

“Halogen” or “Halo” refers to fluoro, chloro, bromo and/or iodo. Where a residue is substituted with more than one halogen, it can be referred to by using a prefix corresponding to the number of halogen substituent attached, e.g., dihaloaryl, dihaloalkyl, trihaloaryl etc. refer to aryl and alkyl substituted with two (“di”) or three (“tri”) Halogen groups, which can be but are not necessarily the same Halogen; thus 4-chloro-3-fluorophenyl is within the scope of dihaloaryl. An alkyl group in which one or more hydrogen is replaced with a Halogen group is referred to as a “haloalkyl”, for example, “C₁₋₆ haloalkyl.” A preferred haloalkyl group is trifluoroalkyl (—CF₃).

“Heteroaryl” refers to an aromatic heterocyclic mono-, bi- or tricyclic ring system of 5 to 14 ring atoms, preferably from 5 to 10 ring atoms, more preferably from 5 to 6 ring atoms, comprising 1, 2, 3 or 4 heteroatoms selected from N, O and S, the remaining ring atoms being carbon. In some aspects, monocyclic heteroaryl rings may be 5-6 membered. Bicyclic heteroaryl ring systems include fused bicyclics having two fused five-membered heteroaryl rings (denoted as 5-5), having a five-membered heteroaryl ring and a fused six-membered heteroaryl ring (denoted as 5-6 and 6-5), and having two fused six-membered heteroaryl rings (denoted as 6-6). The heteroaryl group can be optionally substituted as defined herein. Examples of heteroaryl substituent include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, isoxazolyl, benzofuranyl, isothiazolyl, benzothienyl, benzothiophenyl, indolyl, aza-indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, pyrrolopyridinyl, furopyridinyl, thienopyridinyl, pyrrolopyridazinyl, pyrrolopyrimidinyl, pyrrolopyrazinyl, thienopyridazinyl, thienopyrimidinyl, thienopyrazinyl, furopyridazinyl, furopyrimidinyl, and furopyrazinyl. Most preferably, “5-membered heteroaryl” refers to the following groups:

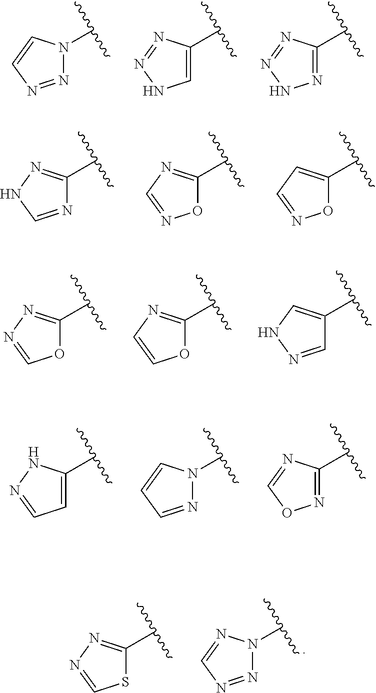

“Heterocycle” or “heterocyclyl” refers to a 3, 4, 5, 6, 7, 8, 9, 10-membered monocyclic, 7, 8, 9 and 10-membered bicyclic (including bridged bicyclic and cycloalkyl spiro substituent) or 10, 11, 12, 13, 14 and 15-membered bicyclic heterocyclic moiety that is saturated or partially unsaturated, and has one or more (e.g., 1, 2, 3 or 4 heteroatoms selected from oxygen, nitrogen and sulfur in the ring with the remaining ring atoms being carbon. In some aspects, the heterocycle is a heterocycloalkyl. In particular aspects heterocycle or heterocyclyl refers to a 4, 5, 6 or 7-membered heterocycle. When used in reference to a ring atom of a heterocycle, a nitrogen or sulfur may also be in an oxidized form, and a nitrogen may be substituted with one or more (C₁-C₆)alkyl or groups. The heterocycle can be attached to its pendant group at any heteroatom or carbon atom that results in a stable structure. Any of the heterocycle ring atoms can be optionally substituted with one or more substituents described herein. Examples of such saturated or partially unsaturated heterocyclyl include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, pyrrolidine 1-oxide, N-hydroxypiperidine, 1-methylpyrrolidine N-oxide, diazirinyl and quinuclidinyl. The term heterocycle also includes groups in which a heterocycle is fused to one or more aryl, heteroaryl, or cycloalkyl rings, such as indolinyl, 3H-indolyl, chromanyl, azabicyclo[2.2.1]heptanyl, azabicyclo[3.1.0]hexanyl, azabicyclo[3.1.1]heptanyl, octahydroindolyl, or tetrahydroquinolinyl.

“Hydroxy”, alone or in combination with other groups, refers to OH.

“Hydroxyalkyl” refers to an alkyl group wherein one or more of the hydrogen atoms of the alkyl group have been replaced by a hydroxy moiety. Examples include alcohols and diols.

“Moiety” and “substituent” refer to an atom or group of chemically bonded atoms that is attached to another atom or molecule by one or more chemical bonds thereby forming part of a molecule.

When indicating the number of substituents, the term “one or more” refers to the range from one substituent to the highest possible number of substitution, i.e. replacement of one hydrogen up to replacement of all hydrogens by substituents, in particular wherein “one or more” refers to one, two or three, most particularly “one or more” refers to one or two.

“Optional” or “Optionally” means that the subsequently described event or circumstance may but need not occur, and that the description includes instances where the event or circumstance occurs and instances in which it does not. For example, “aryl group optionally substituted with an alkyl group” means that the alkyl may but need not be present, and the description includes situations where the aryl group is substituted with an alkyl group and situations where the aryl group is not substituted with the alkyl group.

“Optionally substituted” refers to means unsubstituted or substituted. Generally these substituents can be the same or different.

“Oxo”, alone or in combination with other groups, refers to ═O.

“Pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein.

Particularly preferred pharmaceutically acceptable salts of compounds of formula (I) are the salts of hydrochloric acid, hydrobromic acid, sulfuric acid, phosphoric acid and methanesulfonic acid.

“Protecting group” (PG) denotes the group which selectively blocks a reactive site in a multifunctional compound such that a chemical reaction can be carried out selectively at another unprotected reactive site in the meaning conventionally associated with it in synthetic chemistry. Protective groups can be removed at the appropriate point. Exemplary protective groups are amino-protective groups, carboxy-protective groups or hydroxy-protective groups. Particular protective groups are the tert-butoxycarbonyl (Boc), benzyloxycarbonyl (Cbz), fluorenylmethoxycarbonyl (Fmoc) and benzyl (Bn). Further particular protective groups are the tert-butoxycarbonyl (Boc) and the fluorenylmethoxycarbonyl (Fmoc). More particular protective group is the tert-butoxycarbonyl (Boc). Exemplary protective groups and their application in organic synthesis are described, for example, in “Protective Groups in Organic Chemistry” by T. W. Greene and P. G. M. Wutts, 5th Ed., 2014, John Wiley & Sons, N.Y.

“Prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

“Substituted” refers to the replacement of at least one of hydrogen atoms of a compound or moiety with another substituent or moiety. Examples of such substituents include, without limitation, halogen, —OH, —CN, oxo, alkoxy, alkyl, alkylene, aryl, heteroaryl, haloalkyl, haloalkoxy, cycloalkyl and heterocycle. For example, the term “haloalkyl” refers to the fact that one or more hydrogen atoms of an alkyl (as defined below) is replaced by one or more halogen atoms (e.g., trifluoromethyl, difluoromethyl, fluoromethyl, chloromethyl, etc.). In one aspect, substituted as used herein can refer to replacement of at least one hydrogen atom of a compound or moiety described herein with halogen or alkyl.

“Therapeutically effective amount” refers to an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

“Therapeutically inert carrier” refers to any ingredient having no therapeutic activity and being non-toxic such as disintegrators, binders, fillers, solvents, buffers, tonicity agents, stabilizers, antioxidants, surfactants or lubricants used in formulating pharmaceutical products.

In particular, the chemical groups whose definitions are given above are those specifically exemplified in the examples.

The following abbreviations are used in the present text: AIBN=2,2-azobis(2-methylpropionitrile), BOP=benzotriazol-1-yloxytris(dimethylamino)phosphonium hexafluorophosphate, Brine=saturated aqueous NaCl solution, CAS=chemical abstracts registration number, CDI=1,1′-Carbonyldiimidazole, DBU=1,8-diazabicyclo[5,4,0]undec-7-ene, DCM=dichloromethane, DDQ=2,3-dichloro-5,6-dicyano-1,4-benzoquinone, DMF=N,N-dimethylformamide, DIPEA=N,N-diisopropylethylamine, EDC=1-ethyl-3-(3-dimethylaminopropyl)carbodiimide, ESI=electrospray ionization, EtOAc=ethyl acetate, EtOH=ethanol, h=hour(s), HATU=1-[bis(dimethylamino)methylene]-1H-1,2,3-triazolo[4,5-b]pyridinium-3-oxide hexafluorophosphate, HBTU=O-benzotriazole-N,N,N′,N′-tetramethyl-uronium-hexafluorophosphate, HFIP=hexafluoroisopropanol, HOBt=hydroxybenzotriazole, HPLC=high performance liquid chromatography, m-CPBA=meta-chloroperoxybenzoic acid, MeCN=acetonitrile, Mel=methyliodide, MeOH=methanol, min=minute(s), MS=mass spectrum, NBS=N-bromosuccinimide, PE=petroleum ether, PyBroP=bromo-tris-pyrrolidino-phosphonium hexafluorophosphate, RT=room temperature, RP=reverse phase, TBAF=tetrabutylammonium fluoride, TBAOH=tetrabutylammonium hydroxide, TBDMS=tert-butyldimethylsilyl, TEA=triethylamine, TFA=trifluoroacetic acid, THF=tetrahydrofuran, TMSOTF=trifluoromethanesulfonic acid trimethylsilylester, TLC=thin layer chromatography, T₃P=2,4,6-tripropyl-1,3,5,2,4,6-trioxatriphosphinane 2,4,6-trioxide.

In the description herein, if there is a discrepancy between a depicted structure and a name given to that structure, then the depicted structure controls. Additionally, if the stereochemistry of a structure or a portion of a structure is not indicated with, for example, bold wedged, or dashed lines, the structure or portion of the structure is to be interpreted as encompassing all stereoisomers of it. In some cases, however, where more than one chiral center exists, the structures and names may be represented as single enantiomers to help describe the relative stereochemistry.

Unless otherwise indicated, the terms “a compound of the formula” or “a compound of formula” or “compounds of the formula” or “compounds of formula” refer to any compound selected from the genus of compounds as defined by the formula (including any pharmaceutically acceptable salt of any such compound if not otherwise noted).

Certain compounds may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—↔—C(—OH)═CH—), amide/imidic acid (—C(═O)—NH—↔—C(—OH)═N—) and amidine (—C(═NR)—NH—↔—C(—NHR)═N—) tautomers. The latter two are particularly common in heteroaryl and heterocyclic rings and the present invention encompasses all tautomeric forms of the compounds.

Furthermore, the invention includes all optical isomers, i.e. diastereoisomers, diastereomeric mixtures, racemic mixtures, all their corresponding enantiomers and/or tautomers as well as their solvates of the compounds of formula (I).

The compounds of formula (I) may contain one or more asymmetric centers and can therefore occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within this invention. The present invention is meant to encompass all such isomeric forms of these compounds. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where optically pure enantiomers are provided, optically pure enantiomer means that the compound contains >90% of the desired isomer by weight, particularly >95% of the desired isomer by weight, or more particularly >99% of the desired isomer by weight, said weight percent based upon the total weight of the isomer(s) of the compound. Chirally pure or chirally enriched compounds may be prepared by chirally selective synthesis or by separation of enantiomers. The separation of enantiomers may be carried out on the final product or alternatively on a suitable intermediate.

In some embodiments, the compounds of formula (I) are isotopically-labeled by having one or more atoms therein replaced by an atom having a different atomic mass or mass number.

Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Compounds of the Invention

In one embodiment, there is provided a compound of formula (I),

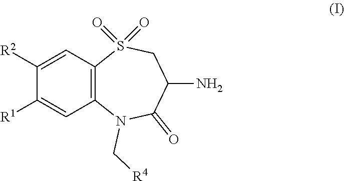

or a pharmaceutically acceptable salt thereof, wherein:
R¹ is oxadiazole, wherein R¹ is optionally substituted with one or more R¹⁰ which can be the same or different;
R² is selected from hydrogen and halogen;
R⁴ is selected from C₅₋₁₄-aryl and 5-14 membered heteroaryl, wherein R⁴ is optionally substituted with one or more R¹¹ which can be the same or different;
R¹⁰ is selected from:
   i) C₁₋₁₀-alkyl, optionally substituted with one or more halogen, amino, hydroxy, C₁₋₆-alkoxy, 3-10 membered cycloalkyl, phenyl, cyano;
   ii) C₃₋₁₀-cycloalkyl, optionally substituted with one or more halogen, cyano, amino;
   iii) 3-10 membered heterocyclyl, optionally substituted with one or more halogen, C₁₋₁₀-alkyl, amino, halo-C₁₋₆-alkyl, hydroxy, cyano, —C(O)O—(R^{10q}), C₃₋₁₀-cycloalkyl, wherein C₁₋₁₀-alkyl is optionally substituted with one or more hydroxy, C₁₋₆-alkoxy;
   iv) —N(R^{10e}R^{10f});
   v) heteroaryl, optionally substituted with one or more C₁₋₁₀-alkyl, halogen;
R^{10e} and R^{10f} are each independently selected from:
   i) hydrogen;
   ii) C₁₋₆-alkyl, optionally substituted with one or more, cyano, halogen, hydroxy;
   iii) C₃₋₁₀-cycloalkyl, optionally substituted with one or more halogen, C₁₋₁₀-alkyl;
   R^{10q} is C₁₋₅-alkyl, wherein C₁₋₅-alkyl is optionally substituted with one or more hydroxy;
R¹¹ is selected from:
   i) 5-6 membered heteroaryl, optionally substituted with one or more C₁₋₆-alkyl, C₃₋₁₀ cycloalkyl, halo-C₁₋₆-alkyl, C₁₋₆-alkoxy, halo-C₁₋₆-alkoxy, wherein C₃₋₁₀ cycloalkyl is optionally substituted with one or more halogen;
   ii) phenyl, optionally substituted with one or more C₁₋₆-alkoxy, —OH, halo-C₁₋₆-alkyl.

In another embodiment, there is provided a compound of formula (I) as described herein, or a pharmaceutically acceptable salt thereof, wherein R¹¹ is selected from:
(i) 5-6 membered heteroaryl, optionally substituted with one or more C₁₋₆-alkyl, C₃₋₁₀ cycloalkyl, halo-C₁₋₆-alkyl;
(ii) phenyl, optionally substituted with one or more C₁₋₆-alkoxy, halo-C₁₋₆-alkyl, halo-C₁₋₆-alkoxy.

In another embodiment, there is provided a compound of formula (I) as described herein, or a pharmaceutically acceptable salt thereof, wherein R² is selected from hydrogen and fluorine.

In another embodiment, there is provided a compound of formula (I) as described herein, or a pharmaceutically acceptable salt thereof, wherein R⁴ is selected from phenyl and pyridinyl, wherein R⁴ is optionally substituted with one or more R¹¹ which can be the same or different.

In another embodiment, there is provided a compound of formula (I) as described herein, or a pharmaceutically acceptable salt thereof, wherein R¹⁰ is tert-butyl, pyrrolidinyl, tetrafluoro-methoxy-ethyl, methyl-propanenitrile, difluoromorpholinyl, oxa-azaspiro[2.5]octan-yl, (trifluoromethyl)morpholinyl, aminocyclohexyl, cyclopropanecarbonitrile, difluoro-piperidyl, ethoxy-tetrafluoro-ethyl, (hydroxymethyl)tetrahydrofuranyl, azabicyclo[3.1.1]heptane-methylcarboxylate, amino-trifluoromethyl-ethyl, difluoro-piperidine-methylcarboxylate, fluoro-methyl-piperidyl, aminooxetanyl, (difluoro-methyl-cyclobutyl)aminoyl, cyclopropyltetrahydrofuranyl, amino-dimethyl-propyl, propanenitrile, isopropylaminoyl, fluoro-methyl-pyridyl, methyl-pyridyl, chloro-pyridyl, tetrafluoroethyl, trifluoro-dihydroxy-ethyl, hydroxy-(trifluoromethyl)propyl, pentafluoroethyl, trifluoro-dimethyl-ethyl, trifluoro-phenyl-ethyl, benzyl-trifluoroethyl, (trifluoromethyl)oxetanyl, trifluoro(hydroxymethyl)ethyl, amino-cyclopropyl-trifluoro-ethyl, trifluoro-hydroxy-methyl-ethyl, trifluoroethyl, morpholino, hexahydro-2H-pyrano[4,3-b]pyrrolyl, hexahydro-2H-cyclopenta[b][1,4]oxazinyl, dioxaazabicyclo[3.3.1]nonanyl, morpholinyl-carbonitrile, (methoxymethyl)morpholinyl, (hydroxymethyl)morpholinyl, (hydroxyethyl)morpholinyl, oxazepanyl, difluoro-(methoxyethyl)-piperidyl, aminocyclohexyl, amino-trifluoro-methyl-ethyl, methyloxetanyl, trifluoro-hydroxy-(trifluoromethyl)ethyl, (trifluoromethyl)oxetan-3-yl, trifluoro-(hydroxymethyl)ethyl, amino-trifluoro-methyl-ethyl, hexahydrofuro[3,2-b]pyrrolyl, difluoro-azabicyclo[4.1.0]heptanyl, hexahydrofuro[2,3-b][1,4]oxazinyl, hexahydro-2H-cyclopenta[b][1,4]oxazinyl, hexahydro-2H-pyrano[4,3-b][1,4]oxazinyl, hexahydro-2H-cyclopenta[b][1,4]oxazinyl, oxa-azabicyclo[3.2.1]octanyl, cyclopropyl-difluoro-tetrahydrofuranyl, difluorocyclohexyl, amino-trifluoro-ethyl, difluoroethyl(hydroxyethyl)amino, difluoroethyl-aminoyl-acetonitrile, cyclopropyl(difluoroethyl)amino, difluoropyrrolidinyl, (trifluoro-methyl-ethyl)amino, trifluoroethylamino, methyl(trifluoroethyl)amino, ethyl-difluoro-piperidyl, dimethyl-pyridyl, trifluoro-methoxy-ethyl.

In another embodiment, there is provided a compound of formula (I) as described herein, or a pharmaceutically acceptable salt thereof, wherein R¹⁰ is tert-butyl, tetrafluoro-methoxy-ethyl, methyl-propanenitrile, difluoromorpholinyl, oxa-azaspiro[2.5]octan-yl, (trifluoromethyl)morpholinyl, cyclopropanecarbonitrile, difluoro-piperidyl, (hydroxymethyl)tetrahydrofuranyl, amino-trifluoromethyl-ethyl, aminooxetanyl, cyclopropyltetrahydrofuranyl, propanenitrile, aminocyclohexyl.

In another embodiment, there is provided a compound of formula (I) as described herein, or a pharmaceutically acceptable salt thereof, wherein R¹¹ is selected from (hydroxymethyl)phenyl, (trifluoromethyl)oxadiazolyl, cyclopropyl-oxadiazolyl, (trifluoromethyl)pyridyl, (trifluoromethyl)phenyl, methoxyphenyl, (trifluoromethyl)pyridyl, dimethylpyrazolyl, tert-butyl-oxadiazolyl, methyl-oxadiazolyl, methylpyrazolyl, (difluoromethyl)-oxadiazolyl, (trifluoromethyl)oxazolyl, methyl-(trifluoromethyl)pyrazolyl, (trifluoromethyl)pyrazolyl, (trifluoromethyl)isoxazolyl, (trifluoromethyl-ethyl)oxadiazolyl, (trifluoroethyl)oxadiazolyl, (trifluoromethoxy)phenyl, cyclopropyl-triazolyl, (trifluoromethoxy)pyridyl, (trifluoromethoxy)pyrimidinyl, (pentafluoroethoxy)pyridyl, (trifluoromethoxy), pyridyl, methyl-(trifluoromethoxy)pyrazolyl.

In another embodiment, there is provided a compound of formula (I) as described herein, or a pharmaceutically acceptable salt thereof, wherein R¹¹ is selected from (hydroxymethyl)phenyl, (trifluoromethyl)oxadiazolyl, cyclopropyl-oxadiazolyl, (trifluoromethyl)pyridyl, (trifluoromethyl)phenyl, methoxyphenyl, (trifluoromethyl)pyridyl, dimethylpyrazolyl, tert-butyl-oxadiazolyl, methyl-oxadiazolyl, methylpyrazolyl, (difluoromethyl)-oxadiazolyl, (trifluoromethyl)oxazolyl, methyl-(trifluoromethyl)pyrazolyl, (trifluoromethyl)pyrazolyl, (trifluoromethyl)isoxazolyl, (trifluoromethyl-ethyl)oxadiazolyl, (trifluoroethyl)oxadiazolyl, (trifluoromethoxy)phenyl, cyclopropyl-triazolyl.

In another embodiment, there is provided a compound of formula (I) as described herein, or a pharmaceutically acceptable salt thereof, wherein R¹¹ is selected from (trifluoromethyl)oxadiazolyl, cyclopropyl-oxadiazolyl, (trifluoromethyl)pyridyl, methoxyphenyl, (trifluoromethoxy)pyridyl, (trifluoromethoxy)pyrimidinyl, (pentafluoroethoxy)pyridyl, (trifluoromethoxy), pyridyl, methyl-(trifluoromethoxy)pyrazolyl.

In another embodiment, there is provided a compound of formula (I) as described herein, or a pharmaceutically acceptable salt thereof, wherein R¹¹ is selected from (trifluoromethyl)oxadiazolyl, cyclopropyl-oxadiazolyl, (trifluoromethyl)pyridyl, methoxyphenyl.

In another embodiment, there is provided a compound of formula (I) as described herein, or a pharmaceutically acceptable salt thereof, wherein:
R¹ is oxadiazole, wherein R¹ is optionally substituted with one or more R¹⁰ which can be the same or different;
R² is selected from hydrogen and fluorine;
R⁴ is selected from phenyl and pyridinyl, wherein R⁴ is optionally substituted with one or more R¹¹ which can be the same or different;
R¹⁰ is selected from:
   i) C₁₋₁₀-alkyl, optionally substituted with one or more halogen, amino, hydroxy, C₁₋₆-alkoxy, 3-10 membered cycloalkyl, phenyl, cyano;
   ii) C₃₋₁₀-cycloalkyl, optionally substituted with one or more halogen, cyano, amino;
   iii) 3-10 membered heterocyclyl, optionally substituted with one or more halogen, C₁₋₁₀-alkyl, amino, halo-C₁₋₆-alkyl, hydroxy, cyano, —C(O)O—(R^{10q}), C₃₋₁₀-cycloalkyl, wherein C₁₋₁₀-alkyl is optionally substituted with one or more hydroxy, C₁₋₆-alkoxy;
   iv) —N(R^{10e}R^{10f});
   v) heteroaryl, optionally substituted with one or more C₁₋₁₀-alkyl, halogen;
      R^{10e} and R^{10f} are each independently selected from:
   i) hydrogen;
   ii) C₁₋₆-alkyl, optionally substituted with one or more, cyano, halogen, hydroxy;
   iii) C₃₋₁₀-cycloalkyl, optionally substituted with one or more halogen, C₁₋₁₀-alkyl;
      R^{10q} is C₁₋₅-alkyl, wherein C₁₋₅-alkyl is optionally substituted with one or more hydroxy;
R¹¹ is selected from:
   i) 5-6 membered heteroaryl, optionally substituted with one or more C₁₋₆-alkyl, C₃₋₁₀ cycloalkyl, halo-C₁₋₆-alkyl;
   ii) phenyl, optionally substituted with one or more C₁₋₆-alkoxy, halo-C₁₋₆-alkyl, halo-C₁₋₆-alkoxy.

In another embodiment, there is provided a compound of formula (I) as described herein, or a pharmaceutically acceptable salt thereof, wherein:
R¹ is oxadiazole, wherein R¹ is optionally substituted with one or more R¹⁰ which can be the same or different;
R² is selected from hydrogen and fluorine;
R⁴ is selected from phenyl and pyridine, wherein R⁴ is optionally substituted with one or more R¹¹ which can be the same or different;
R¹⁰ is tert-butyl, pyrrolidinyl, tetrafluoro-methoxy-ethyl, methyl-propanenitrile, difluoromorpholinyl, oxa-azaspiro[2.5]octan-yl, (trifluoromethyl)morpholinyl, aminocyclohexyl, cyclopropanecarbonitrile, difluoro-piperidyl, ethoxy-tetrafluoro-ethyl, (hydroxymethyl)tetrahydrofuranyl, azabicyclo[3.1.1]heptane-methylcarboxylate, amino-trifluoromethyl-ethyl, difluoro-piperidine-methylcarboxylate, fluoro-methyl-piperidyl, aminooxetanyl, (difluoro-methyl-cyclobutyl)aminoyl, cyclopropyltetrahydrofuranyl, amino-dimethyl-propyl, propanenitrile, isopropylaminoyl, fluoro-methyl-pyridyl, methyl-pyridyl, chloro-pyridyl, tetrafluoroethyl, trifluoro-dihydroxy-ethyl, hydroxy-(trifluoromethyl)propyl, pentafluoroethyl, trifluoro-dimethyl-ethyl, trifluoro-phenyl-ethyl, benzyl-trifluoroethyl, (trifluoromethyl)oxetanyl, trifluoro(hydroxymethyl)ethyl, amino-cyclopropyl-trifluoro-ethyl, trifluoro-hydroxy-methyl-ethyl, trifluoroethyl, morpholino, hexahydro-2H-pyrano[4,3-b]pyrrolyl, hexahydro-2H-cyclopenta[b][1,4]oxazinyl, dioxaazabicyclo[3.3.1]nonanyl, morpholinyl-carbonitrile, (methoxymethyl)morpholinyl, (hydroxymethyl)morpholinyl, (hydroxyethyl)morpholinyl, oxazepanyl, difluoro-(methoxyethyl)-piperidyl, aminocyclohexyl, amino-trifluoro-methyl-ethyl, methyloxetanyl, trifluoro-hydroxy-(trifluoromethyl)ethyl, (trifluoromethyl)oxetan-3-yl, trifluoro-(hydroxymethyl)ethyl, amino-trifluoro-methyl-ethyl, hexahydrofuro[3,2-b]pyrrolyl, difluoro-azabicyclo[4.1.0]heptanyl, hexahydrofuro[2,3-b][1,4]oxazinyl, hexahydro-2H-cyclopenta[b][1,4]oxazinyl, hexahydro-2H-pyrano[4,3-b][1,4]oxazinyl, hexahydro-2H-cyclopenta[b][1,4]oxazinyl, oxa-azabicyclo[3.2.1]octanyl, cyclopropyl-difluoro-tetrahydrofuranyl, difluorocyclohexyl, amino-trifluoro-ethyl, difluoroethyl(hydroxyethyl)amino, difluoroethyl-aminoyl-acetonitrile, cyclopropyl(difluoroethyl)amino, difluoropyrrolidinyl, (trifluoro-methyl-ethyl)amino, trifluoroethylamino, methyl(trifluoroethyl)amino, ethyl-difluoro-piperidyl, dimethyl-pyridyl, trifluoro-methoxy-ethyl;
R¹¹ is selected from (hydroxymethyl)phenyl, (trifluoromethyl)oxadiazolyl, cyclopropyl-oxadiazolyl, (trifluoromethyl)pyridyl, (trifluoromethyl)phenyl, methoxyphenyl, (trifluoromethyl)pyridyl, dimethylpyrazolyl, tert-butyl-oxadiazolyl, methyl-oxadiazolyl, methylpyrazolyl, (difluoromethyl)-oxadiazolyl, (trifluoromethyl)oxazolyl, methyl-(trifluoromethyl)pyrazolyl, (trifluoromethyl)pyrazolyl, (trifluoromethyl)isoxazolyl, (trifluoromethyl-ethyl)oxadiazolyl, (trifluoroethyl)oxadiazolyl, (trifluoromethoxy)phenyl, cyclopropyl-triazolyl.

In another embodiment, there is provided a compound of formula (I) as described herein, or a pharmaceutically acceptable salt thereof, wherein:
R¹ is oxadiazole, wherein R¹ is optionally substituted with one or more R¹⁰ which can be the same or different;
R² is selected from hydrogen and fluorine;
R⁴ is selected from phenyl and pyridine, wherein R⁴ is optionally substituted with one or more R¹¹ which can be the same or different;
R¹⁰ is tert-butyl, tetrafluoro-methoxy-ethyl, methyl-propanenitrile, difluoromorpholinyl, oxa-azaspiro[2.5]octan-yl, (trifluoromethyl)morpholinyl, cyclopropanecarbonitrile, difluoro-piperidyl, (hydroxymethyl)tetrahydrofuranyl, amino-trifluoromethyl-ethyl, aminooxetanyl, cyclopropyltetrahydrofuranyl, propanenitrile, aminocyclohexyl;
R¹¹ is selected from (trifluoromethyl)oxadiazolyl, cyclopropyl-oxadiazolyl, (trifluoromethyl)pyridyl, methoxyphenyl.

In another embodiment, there is provided a compound of formula (I) as described herein, or a pharmaceutically acceptable salt thereof, wherein:
R¹ is oxadiazole, wherein R¹ is optionally substituted with one or more R¹⁰ which can be the same or different;
R² is selected from hydrogen and fluorine;
R⁴ is selected from phenyl and pyridinyl, wherein R⁴ is optionally substituted with one or more R¹¹ which can be the same or different;
R¹⁰ is tert-butyl, pyrrolidinyl, tetrafluoro-methoxy-ethyl, methyl-propanenitrile, difluoromorpholinyl, oxa-azaspiro[2.5]octan-yl, (trifluoromethyl)morpholinyl, aminocyclohexyl, cyclopropanecarbonitrile, difluoro-piperidyl, ethoxy-tetrafluoro-ethyl, (hydroxymethyl)tetrahydrofuranyl, azabicyclo[3.1.1]heptane-methylcarboxylate, amino-trifluoromethyl-ethyl, difluoro-piperidine-methylcarboxylate, fluoro-methyl-piperidyl, aminooxetanyl, (difluoro-methyl-cyclobutyl)aminoyl, cyclopropyltetrahydrofuranyl, amino-dimethyl-propyl, propanenitrile, isopropylaminoyl, fluoro-methyl-pyridyl, methyl-pyridyl, chloro-pyridyl, tetrafluoroethyl, trifluoro-dihydroxy-ethyl, hydroxy-(trifluoromethyl)propyl, pentafluoroethyl, trifluoro-dimethyl-ethyl, trifluoro-phenyl-ethyl, benzyl-trifluoroethyl, (trifluoromethyl)oxetanyl, trifluoro(hydroxymethyl)ethyl, amino-cyclopropyl-trifluoro-ethyl, trifluoro-hydroxy-methyl-ethyl, trifluoroethyl, morpholino, hexahydro-2H-pyrano[4,3-b]pyrrolyl, hexahydro-2H-cyclopenta[b][1,4]oxazinyl, dioxaazabicyclo[3.3.1]nonanyl, morpholinyl-carbonitrile, (methoxymethyl)morpholinyl, (hydroxymethyl)morpholinyl, (hydroxyethyl)morpholinyl, oxazepanyl, difluoro-(methoxyethyl)-piperidyl, aminocyclohexyl, amino-trifluoro-methyl-ethyl, methyloxetanyl, trifluoro-hydroxy-(trifluoromethyl)ethyl, (trifluoromethyl)oxetan-3-yl, trifluoro-(hydroxymethyl)ethyl, amino-trifluoro-methyl-ethyl, hexahydrofuro[3,2-b]pyrrolyl, difluoro-azabicyclo[4.1.0]heptanyl, hexahydrofuro[2,3-b][1,4]oxazinyl, hexahydro-2H-cyclopenta[b][1,4]oxazinyl, hexahydro-2H-pyrano[4,3-b][1,4]oxazinyl, hexahydro-2H-cyclopenta[b][1,4]oxazinyl, oxa-azabicyclo[3.2.1]octanyl, cyclopropyl-difluoro-tetrahydrofuranyl, difluorocyclohexyl, amino-trifluoro-ethyl, difluoroethyl(hydroxyethyl)amino, difluoroethyl-aminoyl-acetonitrile, cyclopropyl(difluoroethyl)amino, difluoropyrrolidinyl, (trifluoro-methyl-ethyl)amino, trifluoroethylamino, methyl(trifluoroethyl)amino, ethyl-difluoro-piperidyl, dimethyl-pyridyl, trifluoro-methoxy-ethyl;
R¹¹ is selected from (hydroxymethyl)phenyl, (trifluoromethyl)oxadiazolyl, cyclopropyl-oxadiazolyl, (trifluoromethyl)pyridyl, (trifluoromethyl)phenyl, methoxyphenyl, (trifluoromethyl)pyridyl, dimethylpyrazolyl, tert-butyl-oxadiazolyl, methyl-oxadiazolyl, methylpyrazolyl, (difluoromethyl)-oxadiazolyl, (trifluoromethyl)oxazolyl, methyl-(trifluoromethyl)pyrazolyl, (trifluoromethyl)pyrazolyl, (trifluoromethyl)isoxazolyl, (trifluoromethyl-ethyl)oxadiazolyl, (trifluoroethyl)oxadiazolyl, (trifluoromethoxy)phenyl, cyclopropyl-triazolyl, (trifluoromethoxy)pyridyl, (trifluoromethoxy)pyrimidinyl, (pentafluoroethoxy)pyridyl, (trifluoromethoxy), pyridyl, methyl-(trifluoromethoxy)pyrazolyl.

In another embodiment, there is provided a compound of formula (I) as described herein, or a pharmaceutically acceptable salt thereof, wherein:
R¹ is oxadiazole, wherein R¹ is optionally substituted with one or more R¹⁰ which can be the same or different;
R² is selected from hydrogen and fluorine;
R⁴ is selected from phenyl and pyridinyl, wherein R⁴ is optionally substituted with one or more R¹¹ which can be the same or different;
R¹⁰ is tert-butyl, tetrafluoro-methoxy-ethyl, methyl-propanenitrile, difluoromorpholinyl, oxa-azaspiro[2.5]octan-yl, (trifluoromethyl)morpholinyl, cyclopropanecarbonitrile, difluoro-piperidyl, (hydroxymethyl)tetrahydrofuranyl, amino-trifluoromethyl-ethyl, aminooxetanyl, cyclopropyltetrahydrofuranyl, propanenitrile, aminocyclohexyl;
R¹¹ is selected from (trifluoromethyl)oxadiazolyl, cyclopropyl-oxadiazolyl, (trifluoromethyl)pyridyl, methoxyphenyl, (trifluoromethoxy)pyridyl, (trifluoromethoxy)pyrimidinyl, (pentafluoroethoxy)pyridyl, (trifluoromethoxy), pyridyl, methyl-(trifluoromethoxy)pyrazolyl.

In another embodiment, there is provided a compound of formula (I) as described herein, or a pharmaceutically acceptable salt thereof, selected from:
(3R)-3-amino-7-(5-tert-butyl-1,3,4-oxadiazol-2-yl)-8-fluoro-5-[[4-(1-methylpyrazol-3-yl)phenyl]methyl]-1,1-dioxo-2,3-dihydro-1λ⁶,5-benzothiazepin-4-one
(3R)-3-amino-7-(5-tert-butyl-1,3,4-oxadiazol-2-yl)-8-fluoro-5-[[4-(3-methyl-1,2,4-oxadiazol-5-yl)phenyl]methyl]-1,1-dioxo-2,3-dihydro-1λ⁶,5-benzothiazepin-4-one
2-[5-[(3R)-3-amino-8-fluoro-1,1,4-trioxo-5-[[4-[5-(trifluoromethyl)-2-pyridyl]phenyl]methyl]-2,3-dihydro-1λ⁶,5-benzothiazepin-7-yl]-1,3,4-oxadiazol-2-yl]-2-methyl-propanenitrile
(3R)-3-amino-7-[5-[(3,3-difluoro-1-methyl-cyclobutyl)amino]-1,3,4-oxadiazol-2-yl]-8-fluoro-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-8-fluoro-7-[5-(4-oxa-7-azaspiro[2.5]octan-7-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(4,4-difluoro-1-piperidyl)-1,3,4-oxadiazol-2-yl]-8-fluoro-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-8-fluoro-7-[5-(isopropylamino)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-(5-tert-butyl-1,2,4-oxadiazol-3-yl)-8-fluoro-5-[[6-[4-(hydroxymethyl)phenyl]-3-pyridyl]methyl]-1,1-dioxo-2,3-dihydro-1λ⁶,5-benzothiazepin-4-one
(3R)-3-amino-7-[5-(3-aminooxetan-3-yl)-1,2,4-oxadiazol-3-yl]-8-fluoro-5-[[4-(4-methoxyphenyl)phenyl]methyl]-1,1-dioxo-2,3-dihydro-1λ⁶,5-benzothiazepin-4-one
(3R)-3-amino-7-[5-(3-aminooxetan-3-yl)-1,2,4-oxadiazol-3-yl]-8-fluoro-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(3-aminooxetan-3-yl)-1,2,4-oxadiazol-3-yl]-8-fluoro-1,1-dioxo-5-[[4-[5-(trifluoromethyl)-2-pyridyl]phenyl]methyl]-2,3-dihydro-1λ⁶,5-benzothiazepin-4-one
(3R)-3-amino-8-fluoro-7-[5-(4-oxa-7-azaspiro[2.5]octan-7-yl)-1,2,4-oxadiazol-3-yl]-1,1-dioxo-5-[[6-[4-(trifluoromethyl)phenyl]-3-pyridyl]methyl]-2,3-dihydro-1λ⁶,5-benzothiazepin-4-one
(3R)-3-amino-7-[5-(2,2-difluoromorpholin-4-yl)-1,2,4-oxadiazol-3-yl]-8-fluoro-1,1-dioxo-5-[[6-[5-(trifluoromethyl)-1,2,4-oxadiazol-3-yl]-3-pyridyl]methyl]-2,3-dihydro-1λ⁶,5-benzothiazepin-4-one
(3R)-3-amino-7-[5-(2,2-difluoromorpholin-4-yl)-1,2,4-oxadiazol-3-yl]-8-fluoro-1,1-dioxo-5-[[6-[4-(trifluoromethyl)phenyl]-3-pyridyl]methyl]-2,3-dihydro-1λ⁶,5-benzothiazepin-4-one
(3R)-3-amino-8-fluoro-7-[5-(4-oxa-7-azaspiro[2.5]octan-7-yl)-1,2,4-oxadiazol-3-yl]-1,1-dioxo-5-[[6-[5-(trifluoromethyl)-1,2,4-oxadiazol-3-yl]-3-pyridyl]methyl]-2,3-dihydro-1λ⁶,5-benzothiazepin-4-one
(3R)-3-amino-8-fluoro-1,1-dioxo-7-(5-pyrrolidin-1-yl-1,2,4-oxadiazol-3-yl)-5-[[6-[5-(trifluoromethyl)-1,2,4-oxadiazol-3-yl]-3-pyridyl]methyl]-2,3-dihydro-1λ⁶,5-benzothiazepin-4-one
1-[3-[(3R)-3-amino-8-fluoro-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,2,4-oxadiazol-5-yl]cyclopropanecarbonitrile
1-[3-[(3R)-3-amino-8-fluoro-1,1,4-trioxo-5-[[4-[5-(trifluoromethyl)-2-pyridyl]phenyl]methyl]-2,3-dihydro-1λ⁶,5-benzothiazepin-7-yl]-1,2,4-oxadiazol-5-yl]cyclopropanecarbonitrile
(3R)-3-amino-7-(5-tert-butyl-1,3,4-oxadiazol-2-yl)-5-[[4-(5-tert-butyl-1,3,4-oxadiazol-2-yl)phenyl]methyl]-1,1-dioxo-2,3-dihydro-1λ⁶,5-benzothiazepin-4-one
(3R)-3-amino-7-(5-tert-butyl-1,3,4-oxadiazol-2-yl)-5-[[4-(3-methyl-1,2,4-oxadiazol-5-yl)phenyl]methyl]-1,1-dioxo-2,3-dihydro-1λ⁶,5-benzothiazepin-4-one
(3R)-3-amino-7-(5-tert-butyl-1,3,4-oxadiazol-2-yl)-5-[[4-(1-methylpyrazol-3-yl)phenyl]methyl]-1,1-dioxo-2,3-dihydro-1λ⁶,5-benzothiazepin-4-one
(3R)-3-amino-7-(5-tert-butyl-1,3,4-oxadiazol-2-yl)-5-[[4-(3,5-dimethylpyrazol-1-yl)phenyl]methyl]-1,1-dioxo-2,3-dihydro-1λ⁶,5-benzothiazepin-4-one
(3R)-3-amino-7-(5-tert-butyl-1,3,4-oxadiazol-2-yl)-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-(5-tert-butyl-1,3,4-oxadiazol-2-yl)-1,1-dioxo-5-[[4-[5-(trifluoromethyl)-1,3,4-oxadiazol-2-yl]phenyl]methyl]-2,3-dihydro-1λ⁶,5-benzothiazepin-4-one
(3R)-3-amino-7-(5-tert-butyl-1,3,4-oxadiazol-2-yl)-1,1-dioxo-5-[[4-[4-(trifluoromethyl)pyrazol-1-yl]phenyl]methyl]-2,3-dihydro-1λ⁶,5-benzothiazepin-4-one
(3R)-3-amino-7-(5-tert-butyl-1,3,4-oxadiazol-2-yl)-1,1-dioxo-5-[[4-[3-(trifluoromethyl)-1,2,4-oxadiazol-5-yl]phenyl]methyl]-2,3-dihydro-1λ⁶,5-benzothiazepin-4-one
(3R)-3-amino-7-(5-tert-butyl-1,3,4-oxadiazol-2-yl)-1,1-dioxo-5-[[4-[3-(trifluoromethyl)pyrazol-1-yl]phenyl]methyl]-2,3-dihydro-1λ⁶,5-benzothiazepin-4-one
(3R)-3-amino-7-(5-tert-butyl-1,3,4-oxadiazol-2-yl)-1,1-dioxo-5-[[4-[4-(trifluoromethyl)imidazol-1-yl]phenyl]methyl]-2,3-dihydro-1λ⁶,5-benzothiazepin-4-one
(3R)-3-amino-7-(5-tert-butyl-1,3,4-oxadiazol-2-yl)-5-[[4-(3-cyclopropyl-1,2,4-oxadiazol-5-yl)phenyl]methyl]-1,1-dioxo-2,3-dihydro-1λ⁶,5-benzothiazepin-4-one
(3R)-3-amino-7-(5-tert-butyl-1,3,4-oxadiazol-2-yl)-1,1-dioxo-5-[[4-[5-(trifluoromethyl)isoxazol-3-yl]phenyl]methyl]-2,3-dihydro-1λ⁶,5-benzothiazepin-4-one
(3R)-3-amino-7-(5-tert-butyl-1,3,4-oxadiazol-2-yl)-1,1-dioxo-5-[[4-[3-(trifluoromethyl)isoxazol-5-yl]phenyl]methyl]-2,3-dihydro-1λ⁶,5-benzothiazepin-4-one
(3R)-3-amino-7-(5-tert-butyl-1,3,4-oxadiazol-2-yl)-1,1-dioxo-5-[[4-[4-(trifluoromethyl)oxazol-2-yl]phenyl]methyl]-2,3-dihydro-1λ⁶,5-benzothiazepin-4-one
(3R)-3-amino-7-(5-tert-butyl-1,3,4-oxadiazol-2-yl)-1,1-dioxo-5-[[4-[5-(trifluoromethyl)tetrazol-2-yl]phenyl]methyl]-2,3-dihydro-1λ⁶,5-benzothiazepin-4-one
(3R)-3-amino-7-(5-tert-butyl-1,3,4-oxadiazol-2-yl)-1,1-dioxo-5-[[4-[3-(trifluoromethyl)-1,2,4-triazol-1-yl]phenyl]methyl]-2,3-dihydro-1λ⁶,5-benzothiazepin-4-one
(3R)-3-amino-7-(5-tert-butyl-1,3,4-oxadiazol-2-yl)-5-[[4-[2-methyl-5-(trifluoromethyl)pyrazol-3-yl]phenyl]methyl]-1,1-dioxo-2,3-dihydro-1λ⁶,5-benzothiazepin-4-one
(3R)-3-amino-7-(5-tert-butyl-1,3,4-oxadiazol-2-yl)-1,1-dioxo-5-[[4-[5-(trifluoromethyl)oxazol-2-yl]phenyl]methyl]-2,3-dihydro-1λ⁶,5-benzothiazepin-4-one
(3R)-3-amino-7-[5-(1-amino-2,2,2-trifluoro-1-methyl-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(1-amino-2,2,2-trifluoro-1-methyl-ethyl)-1,3,4-oxadiazol-2-yl]-1,1-dioxo-5-[[4-[5-(2,2,2-trifluoroethyl)-1,2,4-oxadiazol-3-yl]phenyl]methyl]-2,3-dihydro-1λ⁶,5-benzothiazepin-4-one
(3R)-3-amino-7-[5-(1-amino-2,2,2-trifluoro-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-[1-hydroxy-1-(trifluoromethyl)propyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-1,1-dioxo-7-[5-(2,2,2-trifluoro-1-hydroxy-1-methyl-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(2-cyclopropyltetrahydrofuran-2-yl)-1,3,4-oxadiazol-2-yl]-1,1-dioxo-5-[[4-[5-(trifluoromethyl)-1,3,4-oxadiazol-2-yl]phenyl]methyl]-2,3-dihydro-1λ⁶,5-benzothiazepin-4-one (*)
(3R)-3-amino-7-[5-(1-amino-2,2-dimethyl-propyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
2-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]propanenitrile
(3R)-3-amino-7-[5-(2-cyclopropyltetrahydrofuran-2-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-1,1-dioxo-7-[5-(1,2,2,2-tetrafluoro-1-methoxy-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one
2-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]-2-methyl-propanenitrile
(3R)-3-amino-7-[5-(1-aminocyclohexyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
2-[5-[(3R)-3-amino-5-[[4-[5-(difluoromethyl)-2-pyridyl]phenyl]methyl]-1,1,4-trioxo-2,3-dihydro-1λ⁶,5-benzothiazepin-7-yl]-1,3,4-oxadiazol-2-yl]-2-methyl-propanenitrile
(3R)-3-amino-7-[5-(1-aminocyclohexyl)-1,3,4-oxadiazol-2-yl]-1,1-dioxo-5-[[4-[5-(trifluoromethyl)-2-pyridyl]phenyl]methyl]-2,3-dihydro-1λ⁶,5-benzothiazepin-4-one
(3R)-3-amino-7-[5-(1-aminocyclohexyl)-1,3,4-oxadiazol-2-yl]-1,1-dioxo-5-[[4-[4-(trifluoromethyl)phenyl]phenyl]methyl]-2,3-dihydro-1λ⁶,5-benzothiazepin-4-one
(3R)-3-amino-7-[5-(1-aminocyclohexyl)-1,3,4-oxadiazol-2-yl]-5-[[4-(4-methoxyphenyl)phenyl]methyl]-1,1-dioxo-2,3-dihydro-1λ⁶,5-benzothiazepin-4-one
(3R)-3-amino-7-[5-(1-aminocyclohexyl)-1,3,4-oxadiazol-2-yl]-5-[[4-[5-(3,3-difluorocyclopentyl)-1,2,4-oxadiazol-3-yl]phenyl]methyl]-1,1-dioxo-2,3-dihydro-1λ⁶,5-benzothiazepin-4-one
(3R)-3-amino-7-[5-(1-aminocyclohexyl)-1,3,4-oxadiazol-2-yl]-1,1-dioxo-5-[[4-[5-(2,2,2-trifluoro-1-methyl-ethyl)-1,2,4-oxadiazol-3-yl]phenyl]methyl]-2,3-dihydro-1λ⁶,5-benzothiazepin-4-one
2-[5-[(3R)-3-amino-5-[[4-(4-methoxyphenyl)phenyl]methyl]-1,1,4-trioxo-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(trifluoromethyl)-2-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[4-(trifluoromethyl)phenyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[2-(trifluoromethyl)pyrimidin-5-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-5-[[4-[5-(4,4-difluorocyclohexyl)-1,2,4-oxadiazol-3-yl]phenyl]methyl]-1,1,4-trioxo-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[4-(trifluoromethyl)pyrazol-1-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-5-[[4-[5-(4,4-difluoro-1-piperidyl)-1,2,4-oxadiazol-3-yl]phenyl]methyl]-1,1,4-trioxo-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(trifluoromethyl)oxazol-2-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[6-[4-(trifluoromethyl)phenyl]-3-pyridyl]methyl]-2,3-dihydro-1λ⁶,5-benzothiazepin-7-yl]-1,3,4-oxadiazol-2-yl]-2-methyl-propanenitrile (*)
2-[5-[(3R)-3-amino-5-[[4-[2-methyl-5-(trifluoromethyl)pyrazol-3-yl]phenyl]methyl]-1,1,4-trioxo-2,3-dihydro-1λ⁶,5-benzothiazepin-7-yl]-1,3,4-oxadiazol-2-yl]-2-methyl-propanenitrile
2-[5-[(3R)-3-amino-5-[[4-[5-(difluoromethoxy)-2-pyridyl]phenyl]methyl]-1,1,4-trioxo-2,3-dihydro-1λ⁶,5-benzothiazepin-7-yl]-1,3,4-oxadiazol-2-yl]-2-methyl-propanenitrile
2-[5-[(3R)-3-amino-5-[[4-[5-(1,1-difluoroethyl)-2-pyridyl]phenyl]methyl]-1,1,4-trioxo-2,3-dihydro-1λ⁶,5-benzothiazepin-7-yl]-1,3,4-oxadiazol-2-yl]-2-methyl-propanenitrile
2-[5-[(3R)-3-amino-5-[[4-[4-(difluoromethoxy)-2-pyridyl]phenyl]methyl]-1,1,4-trioxo-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[2-(trifluoromethyl)-4-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(trifluoromethoxy)pyrazin-2-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[6-(trifluoromethyl)-3-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-5-[[4-[6-methyl-5-(trifluoromethyl)-2-pyridyl]phenyl]methyl]-1,1,4-trioxo-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[1-(trifluoromethyl)pyrazol-4-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[4-(trifluoromethyl)-2-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(trifluoromethoxy)pyrimidin-2-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-5-[[4-(5-cyclopropyl-2-pyridyl)phenyl]methyl]-1,1,4-trioxo-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[6-[4-(trifluoromethoxy)phenyl]-3-pyridyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(trifluoromethyl)tetrazol-2-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[3-(trifluoromethyl)pyrazol-1-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[4-(trifluoromethyl)oxazol-2-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-5-[[4-(5-methoxy-2-pyridyl)phenyl]methyl]-1,1,4-trioxo-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[4-(trifluoromethoxy)-2-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(trifluoromethyl)pyrimidin-2-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(1,1,2,2,2-pentafluoroethoxy)-2-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(2,2,2-trifluoroethoxy)-2-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[6-(trifluoromethoxy)-3-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[4-(trifluoromethoxy)pyrazol-1-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-5-[[4-(3-cyclopropyl-1,2,4-oxadiazol-5-yl)phenyl]methyl]-1,1,4-trioxo-2,3-dihydro-1λ⁶,5-benzothiazepin-7-yl]-1,3,4-oxadiazol-2-yl]-2-methyl-propanenitrile
2-[5-[(3R)-3-amino-5-[[4-[4-methyl-5-(trifluoromethoxy)-2-pyridyl]phenyl]methyl]-1,1,4-trioxo-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[4-(trifluoromethyl)triazol-1-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-5-[[4-[2-methyl-5-(trifluoromethoxy)pyrazol-3-yl]phenyl]methyl]-1,1,4-trioxo-2,3-dihydro-1λ⁶,5-benzothiazepin-7-yl]-1,3,4-oxadiazol-2-yl]-2-methyl-propanenitrile
(3R)-3-amino-7-[5-(2-methyloxetan-2-yl)-1,3,4-oxadiazol-2-yl]-1,1-dioxo-5-[[4-[4-(trifluoromethyl)phenyl]phenyl]methyl]-2,3-dihydro-1lambda6,5-benzothiazepin-4-one
(3R)-3-amino-7-[5-(3-methyloxetan-3-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(3-methyloxetan-3-yl)-1,3,4-oxadiazol-2-yl]-1,1-dioxo-5-[[4-[5-(trifluoromethyl)-2-pyridyl]phenyl]methyl]-2,3-dihydro-1λ⁶,5-benzothiazepin-4-one
(3R)-3-amino-7-[5-(1-amino-2,2,2-trifluoro-ethyl)-1,3,4-oxadiazol-2-yl]-1,1-dioxo-5-[[4-[4-(trifluoromethyl)phenyl]phenyl]methyl]-2,3-dihydro-1λ⁶,5-benzothiazepin-4-one
(3R)-3-amino-7-[5-[3-(difluoromethyl)azetidin-3-yl]-1,3,4-oxadiazol-2-yl]-1,1-dioxo-5-[[4-[4-(trifluoromethyl)phenyl]phenyl]methyl]-2,3-dihydro-1λ⁶,5-benzothiazepin-4-one
(3R)-3-amino-7-[5-(3-fluoro-1-methyl-3-piperidyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(3-fluoro-1-methyl-3-piperidyl)-1,3,4-oxadiazol-2-yl]-5-[[4-(4-methoxyphenyl)phenyl]methyl]-1,1-dioxo-2,3-dihydro-1λ⁶,5-benzothiazepin-4-one
(3R)-3-amino-7-[5-(3-fluoro-1-methyl-3-piperidyl)-1,3,4-oxadiazol-2-yl]-1,1-dioxo-5-[[4-[5-(trifluoromethyl)-2-pyridyl]phenyl]methyl]-2,3-dihydro-1λ⁶,5-benzothiazepin-4-one
(3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-[(1S)-1-amino-2,2,2-trifluoro-1-methyl-ethyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethoxy)-2-pyridyl]phenyl]methyl]-2,3-dihydro-1λ⁶,5-benzothiazepin-4-one
(3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethyl)-2-pyridyl]phenyl]methyl]-2,3-dihydro-1λ⁶,5-benzothiazepin-4-one
(3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethyl)tetrazol-2-yl]phenyl]methyl]-2,3-dihydro-1λ⁶,5-benzothiazepin-4-one
(3R)-3-amino-7-[5-[(1R)-1-amino-2,2,2-trifluoro-1-methyl-ethyl]-1,3,4-oxadiazol-2-yl]-1,1-dioxo-5-[[4-[3-(trifluoromethyl)pyrazol-1-yl]phenyl]methyl]-2,3-dihydro-1λ⁶,5-benzothiazepin-4-one
(3R)-3-amino-7-[5-[2-(hydroxymethyl)tetrahydrofuran-2-yl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-[2-(hydroxymethyl)tetrahydrofuran-2-yl]-1,3,4-oxadiazol-2-yl]-1,1-dioxo-5-[[4-[4-(trifluoromethyl)phenyl]phenyl]methyl]-2,3-dihydro-1λ⁶,5-benzothiazepin-4-one
methyl 3,3-difluoro-5-[5-[(3R)-3-amino-5-[[4-(5-tert-butyl-1,2,4-oxadiazol-3-yl)phenyl]methyl]-1,1,4-trioxo-2,3-dihydro-1λ⁶,5-benzothiazepin-7-yl]-1,3,4-oxadiazol-2-yl]piperidine-1-carboxylate methyl 3,3-difluoro-5-[5-[(3R)-3-amino-1,1,4-trioxo-5-[[6-[4-(trifluoromethyl)phenyl]-3-pyridyl]methyl]-2,3-dihydro-1λ⁶,5- benzothiazepin-7-yl]-1,3,4-oxadiazol-2-yl]piperidine-1-carboxylate
methyl 1-[5-[(3R)-3-amino-1,1,4-trioxo-5-[[6-[4-(trifluoromethyl)phenyl]-3-pyridyl]methyl]-2,3-dihydro-1λ⁶,5-benzothiazepin-7-yl]-1,3,4-oxadiazol-2-yl]-3-azabicyclo[3.1.1]heptane-3-carboxylate
methyl 1-[5-[(3R)-3-amino-5-[[4-(4-methoxyphenyl)phenyl]methyl]-1,1,4-trioxo-2,3-dihydro-1λ⁶,5-benzothiazepin-7-yl]-1,3,4-oxadiazol-2-yl]-3-azabicyclo[3.1.1]heptane-3-carboxylate
methyl 1-[5-[(3R)-3-amino-1,1,4-trioxo-5-[[4-[4-(trifluoromethyl)phenyl]phenyl]methyl]-2,3-dihydro-1λ⁶,5-benzothiazepin-7-yl]-1,3,4-oxadiazol-2-yl]-3-azabicyclo[3.1.1]heptane-3-carboxylate
methyl 1-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]-3-azabicyclo[3.1.1]heptane-3-carboxylate
1-[5-[(3R)-3-amino-1,1,4-trioxo-5-[[4-[5-(trifluoromethyl)-2-pyridyl]phenyl]methyl]-2,3-dihydro-1λ⁶,5-benzothiazepin-7-yl]-1,3,4-oxadiazol-2-yl]cyclopropanecarbonitrile
methyl 1-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]-3-azabicyclo[3.1.1]heptane-3-carboxylate
methyl 1-[5-[(3R)-3-amino-1,1,4-trioxo-5-[[4-[5-(trifluoromethyl)-2-pyridyl]phenyl]methyl]-2,3-dihydro-1λ⁶,5-benzothiazepin-7-yl]-1,3,4-oxadiazol-2-yl]-3-azabicyclo[3.1.1]heptane-3-carboxylate
(3R)-3-amino-7-[5-[2-(hydroxymethyl)tetrahydrofuran-2-yl]-1,3,4-oxadiazol-2-yl]-5-[[4-(4-methoxyphenyl)phenyl]methyl]-1,1-dioxo-2,3-dihydro-1λ⁶ 5-benzothiazepin-4-one
(3R)-3-amino-7-[5-(1-ethoxy-1,2,2,2-tetrafluoro-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
((3R)-3-amino-5-[[4-(5-cyclopropyl-1,3,4-oxadiazol-2-yl)phenyl]methyl]-1,1-dioxo-7-[5-(1,2,2,2-tetrafluoro-1-methoxy-ethyl)-1,3,4-oxadiazol-2-yl]-2,3-dihydro-1λ⁶,5-benzothiazepin-4-one
(3R)-3-amino-5-[[4-(1-cyclopropyl-1,2,4-triazol-3-yl)phenyl]methyl]-1,1-dioxo-7-[5-(1,2,2,2-tetrafluoro-1-methoxy-ethyl)-1,3,4-oxadiazol-2-yl]-2,3-dihydro-1λ⁶,5-benzothiazepin-4-one
(3R)-3-amino-1,1-dioxo-7-[5-(1,2,2,2-tetrafluoro-1-methoxy-ethyl)-1,3,4-oxadiazol-2-yl]-5-[[4-[5-(trifluoromethyl)-1,3,4-oxadiazol-2-yl]phenyl]methyl]-2,3-dihydro-1λ⁶,5-benzothiazepin-4-one
(3R)-3-amino-7-[5-(4,4-difluoro-1-piperidyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-5-[[4-(5-cyclopropyl-1,2,4-oxadiazol-3-yl)phenyl]methyl]-7-[5-(4,4-difluoro-1-piperidyl)-1,3,4-oxadiazol-2-yl]-1,1-dioxo-2,3-dihydro-1λ⁶,5-benzothiazepin-4-one
(3R)-3-amino-5-[[4-[5-(difluoromethyl)-1,3,4-oxadiazol-2-yl]phenyl]methyl]-7-[5-(4,4-difluoro-1-piperidyl)-1,3,4-oxadiazol-2-yl]-1,1-dioxo-2,3-dihydro-1λ⁶,5-benzothiazepin-4-one
(3R)-3-amino-7-[5-(4,4-difluoro-1-piperidyl)-1,3,4-oxadiazol-2-yl]-1,1-dioxo-5-[[4-[5-(trifluoromethyl)-1,3,4-oxadiazol-2-yl]phenyl]methyl]-2,3-dihydro-1λ⁶,5-benzothiazepin-4-one
(3R)-3-amino-7-[5-(4,4-difluoro-1-piperidyl)-1,3,4-oxadiazol-2-yl]-1,1-dioxo-5-[[4-[5-(trifluoromethyl)-2-pyridyl]phenyl]methyl]-2,3-dihydro-1λ⁶,5-benzothiazepin-4-one
(3R)-3-amino-7-[5-(4,4-difluoro-1-piperidyl)-1,3,4-oxadiazol-2-yl]-1,1-dioxo-5-[[4-[3-(trifluoromethyl)-1,2,4-oxadiazol-5-yl]phenyl]methyl]-2,3-dihydro-1λ⁶,5-benzothiazepin-4-one
(3R)-3-amino-7-[5-(4,4-difluoro-1-piperidyl)-1,3,4-oxadiazol-2-yl]-1,1-dioxo-5-[[4-[3-(trifluoromethyl)pyrazol-1-yl]phenyl]methyl]-2,3-dihydro-1λ⁶,5-benzothiazepin-4-one
4-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]morpholine-2-carbonitrile
(3R)-3-amino-7-[5-[2-(methoxymethyl)morpholin-4-yl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-[(3,3-difluoro-1-methyl-cyclobutyl)amino]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-1,1-dioxo-7-[5-[2-(trifluoromethyl)morpholin-4-yl]-1,3,4-oxadiazol-2-yl]-5-[[4-[4-(trifluoromethyl)phenyl]phenyl]methyl]-2,3-dihydro-1λ⁶,5-benzothiazepin-4-one
(3R)-3-amino-1,1-dioxo-7-[5-[2-(trifluoromethyl)morpholin-4-yl]-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one
(3R)-3-amino-1,1-dioxo-7-[5-[2-(trifluoromethyl)morpholin-4-yl]-1,3,4-oxadiazol-2-yl]-5-[[4-[5-(trifluoromethyl)-2-pyridyl]phenyl]methyl]-2,3-dihydro-1λ⁶,5-benzothiazepin-4-one
(3R)-3-amino-7-[5-(3,3-difluoropyrrolidin-1-yl)-1,3,4-oxadiazol-2-yl]-5-[[4-(4-methoxyphenyl)phenyl]methyl]-1,1-dioxo-2,3-dihydro-1λ⁶,5-benzothiazepin-4-one
(3R)-3-amino-7-[5-(4-oxa-7-azaspiro[2.5]octan-7-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(2,2-difluoromorpholin-4-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(2,2-difluoromorpholin-4-yl)-1,3,4-oxadiazol-2-yl]-5-[[4-(4-methoxyphenyl)phenyl]methyl]-1,1-dioxo-2,3-dihydro-1λ⁶,5-benzothiazepin-4-one
(3R)-3-amino-7-[5-(2,2-difluoromorpholin-4-yl)-1,3,4-oxadiazol-2-yl]-1,1-dioxo-5-[[4-[5-(trifluoromethyl)-1,3,4-oxadiazol-2-yl]phenyl]methyl]-2,3-dihydro-1λ⁶,5-benzothiazepin-4-one
(3R)-3-amino-5-[[4-(4-methoxyphenyl)phenyl]methyl]-7-[5-[methyl(2,2,2-trifluoroethyl)amino]-1,3,4-oxadiazol-2-yl]-1,1-dioxo-2,3-dihydro-1λ⁶,5-benzothiazepin-4-one
(3R)-3-amino-5-[[4-[5-(difluoromethyl)-1,3,4-oxadiazol-2-yl]phenyl]methyl]-7-[5-(2,2-difluoromorpholin-4-yl)-1,3,4-oxadiazol-2-yl]-1,1-dioxo-2,3-dihydro-1λ⁶,5-benzothiazepin-4-one
(3R)-3-amino-7-(5-tert-butyl-1,3,4-oxadiazol-2-yl)-5-[[4-(6-methoxy-3-pyridyl)phenyl]methyl]-1,1-dioxo-2,3-dihydro-1λ⁶,5-benzothiazepin-4-one
(3R)-3-amino-1,1-dioxo-7-[5-(2,2,2-trifluoro-1,1-dimethyl-ethyl)-1,3,4-oxadiazol-2-yl]-5-[[4-[5-(trifluoromethoxy)-2-pyridyl]phenyl]methyl]-2,3-dihydro-1λ⁶,5-benzothiazepin-4-one
(3R)-3-amino-1,1-dioxo-7-[5-(2,2,2-trifluoro-1,1-dimethyl-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one
(3R)-3-amino-5-[[4-(5-cyclopropyl-1,2,4-oxadiazol-3-yl)phenyl]methyl]-1,1-dioxo-7-[5-(2,2,2-trifluoro-1,1-dimethyl-ethyl)-1,3,4-oxadiazol-2-yl]-2,3-dihydro-1λ⁶,5-benzothiazepin-4-one
(3R)-3-amino-5-[[4-(5-cyclopropyl-1,2,4-oxadiazol-3-yl)phenyl]methyl]-1,1-dioxo-7-[5-(1,2,2,2-tetrafluoroethyl)-1,3,4-oxadiazol-2-yl]-2,3-dihydro-1λ⁶,5-benzothiazepin-4-one
(3R)-3-amino-5-[[4-(5-cyclopropyl-1,2,4-oxadiazol-3-yl)phenyl]methyl]-7-[5-[1-hydroxy-1-(trifluoromethyl)propyl]-1,3,4-oxadiazol-2-yl]-1,1-dioxo-2,3-dihydro-1λ⁶,5-benzothiazepin-4-one
(3R)-3-amino-5-[[4-(5-cyclopropyl-1,2,4-oxadiazol-3-yl)phenyl]methyl]-1,1-dioxo-7-[5-(2,2,2-trifluoro-1-hydroxy-1-methyl-ethyl)-1,3,4-oxadiazol-2-yl]-2,3-dihydro-1λ⁶,5-benzothiazepin-4-one
(3R)-3-amino-7-[5-(1-amino-1-cyclopropyl-2,2,2-trifluoro-ethyl)-1,3,4-oxadiazol-2-yl]-5-[[4-(5-cyclopropyl-1,2,4-oxadiazol-3-yl)phenyl]methyl]-1,1-dioxo-2,3-dihydro-1λ⁶,5-benzothiazepin-4-one
1-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]cyclobutanecarbonitrile
4-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]tetrahydropyran-4-carbonitrile
3-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-2,2-dimethyl-propanenitrile
3-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-3-methyl-butanenitrile
2-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-2-ethyl-butanenitrile
3-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]tetrahydrofuran-3-carbonitrile
(3R)-3-amino-7-[5-(1-amino-4,4-difluoro-cyclohexyl)-1,3,4-oxadiazol-2-yl]-1,1-dioxo-5-[[4-[5-(trifluoromethoxy)-2-pyridyl]phenyl]methyl]-2,3-dihydro-1λ⁶,5-benzothiazepin-4-one
(3R)-3-amino-7-[5-(1-amino-3,3-difluoro-cyclobutyl)-1,3,4-oxadiazol-2-yl]-1,1-dioxo-5-[[4-[5-(trifluoromethoxy)-2-pyridyl]phenyl]methyl]-2,3-dihydro-1λ⁶,5-benzothiazepin-4-one
4-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-1-methyl-piperidine-4-carbonitrile
(3R)-3-amino-5-[[4-(5-tert-butyl-1,2,4-oxadiazol-3-yl)phenyl]methyl]-7-[5-(1-ethyl-5,5-difluoro-3-piperidyl)-1,3,4-oxadiazol-2-yl]-1,1-dioxo-2,3-dihydro-1λ⁶,5-benzothiazepin-4-one
(3R)-3-amino-7-[5-[5,5-difluoro-1-(2-methoxyethyl)-3-piperidyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-[5,5-difluoro-1-(2-methoxyethyl)-3-piperidyl]-1,3,4-oxadiazol-2-yl]-5-[[4-(4-methoxyphenyl)phenyl]methyl]-1,1-dioxo-2,3-dihydro-1λ⁶,5-benzothiazepin-4-one
(3R)-3-amino-7-[5-(3-aminooxetan-3-yl)-1,2,4-oxadiazol-3-yl]-1,1-dioxo-5-[[4-[5-(trifluoromethyl)-1,2,4-oxadiazol-3-yl]phenyl]methyl]-2,3-dihydro-1lambda6,5-benzothiazepin-4-one
(3R)-3-amino-7-[5-(2-chloro-3-pyridyl)-1,2,4-oxadiazol-3-yl]-5-[[4-(5-cyclopropyl-1,2,4-oxadiazol-3-yl)phenyl]methyl]-1,1-dioxo-2,3-dihydro-1λ⁶,5-benzothiazepin-4-one
(3R)-3-amino-5-[[4-(5-cyclopropyl-1,2,4-oxadiazol-3-yl)phenyl]methyl]-7-[5-(6-fluoro-2-methyl-3-pyridyl)-1,2,4-oxadiazol-3-yl]-1,1-dioxo-2,3-dihydro-1λ⁶,5-benzothiazepin-4-one
(3R)-3-amino-5-[[4-(5-cyclopropyl-1,2,4-oxadiazol-3-yl)phenyl]methyl]-1,1-dioxo-7-[5-[2-(trifluoromethyl)-3-pyridyl]-1,2,4-oxadiazol-3-yl]-2,3-dihydro-1λ⁶,5-benzothiazepin-4-one
In another embodiment, there is provided a compound of formula (I) as described herein, or a pharmaceutically acceptable salt thereof, selected from:
(3R)-3-amino-8-fluoro-7-[5-(4-oxa-7-azaspiro[2.5]octan-7-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(4,4-difluoro-1-piperidyl)-1,3,4-oxadiazol-2-yl]-8-fluoro-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(3-aminooxetan-3-yl)-1,2,4-oxadiazol-3-yl]-8-fluoro-1,1-dioxo-5-[[4-[5-(trifluoromethyl)-1,2,4-oxadiazol-3-yl]phenyl]methyl]-2,3-dihydro-1λ⁶,5-benzothiazepin-4-one
1-[3-[(3R)-3-amino-8-fluoro-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,2,4-oxadiazol-5-yl]cyclopropanecarbonitrile
1-[3-[(3R)-3-amino-8-fluoro-1,1,4-trioxo-5-[[4-[5-(trifluoromethyl)-2-pyridyl]phenyl]methyl]-2,3-dihydro-1λ⁶,5-benzothiazepin-7-yl]-1,2,4-oxadiazol-5-yl]cyclopropanecarbonitrile
(3R)-3-amino-8-fluoro-7-[5-[2-(hydroxymethyl)tetrahydrofuran-2-yl]-1,2,4-oxadiazol-3-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-(5-tert-butyl-1,3,4-oxadiazol-2-yl)-1,1-dioxo-5-[[4-[5-(trifluoromethyl)-1,3,4-oxadiazol-2-yl]phenyl]methyl]-2,3-dihydro-1λ⁶,5-benzothiazepin-4-one
(3R)-3-amino-7-(5-tert-butyl-1,3,4-oxadiazol-2-yl)-1,1-dioxo-5-[[4-[3-(trifluoromethyl)-1,2,4-oxadiazol-5-yl]phenyl]methyl]-2,3-dihydro-1λ⁶,5-benzothiazepin-4-one
(3R)-3-amino-7-(5-tert-butyl-1,3,4-oxadiazol-2-yl)-5-[[4-(3-cyclopropyl-1,2,4-oxadiazol-5-yl)phenyl]methyl]-1,1-dioxo-2,3-dihydro-1λ⁶,5-benzothiazepin-4-one
(3R)-3-amino-7-[5-(1-amino-2,2,2-trifluoro-1-methyl-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
2-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]propanenitrile
(3R)-3-amino-7-[5-(2-cyclopropyltetrahydrofuran-2-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-1,1-dioxo-7-[5-(1,2,2,2-tetrafluoro-1-methoxy-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one
2-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]-2-methyl-propanenitrile
(3R)-3-amino-7-[5-(1-aminocyclohexyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
2-[5-[(3R)-3-amino-5-[[4-(4-methoxyphenyl)phenyl]methyl]-1,1,4-trioxo-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[4-(trifluoromethyl)phenyl]phenyl]methyl]-2,3-dihydro-1λ⁶,5-benzothiazepin-7-yl]-1,3,4-oxadiazol-2-yl]-2-methyl-propanenitrile
(3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-[(1S)-1-amino-2,2,2-trifluoro-1-methyl-ethyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-[2-(hydroxymethyl)tetrahydrofuran-2-yl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
methyl 1-[5-[(3R)-3-amino-5-[[4-(4-methoxyphenyl)phenyl]methyl]-1,1,4-trioxo-2,3-dihydro-1λ⁶,5-benzothiazepin-7-yl]-1,3,4-oxadiazol-2-yl]-3-azabicyclo[3.1.1]heptane-3-carboxylate
methyl 1-[5-[(3R)-3-amino-1,1,4-trioxo-5-[[4-[4-(trifluoromethyl)phenyl]phenyl]methyl]-2,3-dihydro-1λ⁶,5-benzothiazepin-7-yl]-1,3,4-oxadiazol-2-yl]-3-azabicyclo[3.1.1]heptane-3-carboxylate
methyl 1-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]-3-azabicyclo[3.1.1]heptane-3-carboxylate
(3R)-3-amino-7-[5-(1-ethoxy-1,2,2,2-tetrafluoro-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(4,4-difluoro-1-piperidyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-[(3,3-difluoro-1-methyl-cyclobutyl)amino]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-1,1-dioxo-7-[5-[2-(trifluoromethyl)morpholin-4-yl]-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(2,2-difluoromorpholin-4-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(2,2-difluoromorpholin-4-yl)-1,3,4-oxadiazol-2-yl]-1,1-dioxo-5-[[4-[5-(trifluoromethyl)-1,3,4-oxadiazol-2-yl]phenyl]methyl]-2,3-dihydro-1λ⁶,5-benzothiazepin-4-one
(3R)-3-amino-5-[[4-(4-methoxyphenyl)phenyl]methyl]-7-[5-[methyl(2,2,2-trifluoroethyl)amino]-1,3,4-oxadiazol-2-yl]-1,1-dioxo-2,3-dihydro-1λ⁶,5-benzothiazepin-4-on
(3R)-3-amino-1,1-dioxo-7-[5-(2,2,2-trifluoro-1,1-dimethyl-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one
2-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-2-methyl-propanenitrile
(3R)-3-amino-1,1-dioxo-7-[5-(2,2,2-trifluoro-1-hydroxy-1-methyl-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one
2-[5-[(3R)-3-amino-8-fluoro-1,1,4-trioxo-5-[[4-[5-(trifluoromethyl)-2-pyridyl]phenyl]methyl]-2,3-dihydro-1λ⁶,5-benzothiazepin-7-yl]-1,3,4-oxadiazol-2-yl]-2-methyl-propanenitrile
(3R)-3-amino-1,1-dioxo-7-[5-(2,2,2-trifluoro-1,1-dimethyl-ethyl)-1,3,4-oxadiazol-2-yl]-5-[[4-[5-(trifluoromethoxy)-2-pyridyl]phenyl]methyl]-2,3-dihydro-1λ⁶,5-benzothiazepin-4-one
2-[5-[(3R)-3-amino-1,1,4-trioxo-5-[[4-[5-(trifluoromethoxy)pyrimidin-2-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(1,1,2,2,2-pentafluoroethoxy)-2-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[4-(trifluoromethoxy)pyrazol-1-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-2-ethyl-butanenitrile
2-[5-[(3R)-3-amino-5-[[4-[2-methyl-5-(trifluoromethoxy)pyrazol-3-yl]phenyl]methyl]-1,1,4-trioxo-2,3-dihydro-1λ⁶,5-benzothiazepin-7-yl]-1,3,4-oxadiazol-2-yl]-2-methyl-propanenitrile.

In another embodiment, there is provided a compound of formula (I) as described herein, or a pharmaceutically acceptable salt thereof, selected from:
(3R)-3-amino-8-fluoro-7-[5-(4-oxa-7-azaspiro[2.5]octan-7-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-7-[5-(4,4-difluoro-1-piperidyl)-1,3,4-oxadiazol-2-yl]-8-fluoro-1,1-dioxo-5-[[4-[5-(trifluoromethyl)-1,2,4-oxadiazol-3-yl]phenyl]methyl]-2,3-dihydro-1λ⁶,5-benzothiazepin-4-one
1-[3-[(3R)-3-amino-8-fluoro-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,2,4-oxadiazol-5-yl]cyclopropanecarbonitrile
1-[3-[(3R)-3-amino-8-fluoro-1,1,4-trioxo-5-[[4-[5-(trifluoromethyl)-2-pyridyl]phenyl]methyl]-2,3-dihydro-1λ⁶,5-benzothiazepin-7-yl]-1,2,4-oxadiazol-5-yl]cyclopropanecarbonitrile
(3R)-3-amino-7-(5-tert-butyl-1,3,4-oxadiazol-2-yl)-1,1-dioxo-5-[[4-[3-(trifluoromethyl)-1,2,4-oxadiazol-5-yl]phenyl]methyl]-2,3-dihydro-1λ⁶,5-benzothiazepin-4-one
(3R)-3-amino-7-[5-(2-cyclopropyltetrahydrofuran-2-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-1,1-dioxo-7-[5-(1,2,2,2-tetrafluoro-1-methoxy-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one
2-[5-[(3R)-3-amino-5-[[4-(4-methoxyphenyl)phenyl]methyl]-1,1,4-trioxo-2,3-dihydro-1λ⁶,5-benzothiazepin-7-yl]-1,3,4-oxadiazol-2-yl]-2-methyl-propanenitrile
(3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
methyl 1-[5-[(3R)-3-amino-5-[[4-(4-methoxyphenyl)phenyl]methyl]-1,1,4-trioxo-2,3-dihydro-1λ⁶,5-benzothiazepin-7-yl]-1,3,4-oxadiazol-2-yl]-3-azabicyclo[3.1.1]heptane-3-carboxylate
methyl 1-[5-[(3R)-3-amino-1,1,4-trioxo-5-[[4-[4-(trifluoromethyl)phenyl]phenyl]methyl]-2,3-dihydro-1λ⁶,5-benzothiazepin-7-yl]-1,3,4-oxadiazol-2-yl]-3-azabicyclo[3.1.1]heptane-3-carboxylate
(3R)-3-amino-7-[5-(1-ethoxy-1,2,2,2-tetrafluoro-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-1,1-dioxo-7-[5-(2,2,2-trifluoro-1,1-dimethyl-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one
2-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-2-methyl-propanenitrile
(3R)-3-amino-1,1-dioxo-7-[5-(2,2,2-trifluoro-1-hydroxy-1-methyl-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one
2-[5-[(3R)-3-amino-8-fluoro-1,1,4-trioxo-5-[[4-[5-(trifluoromethyl)-2-pyridyl]phenyl]methyl]-2,3-dihydro-1λ⁶,5-benzothiazepin-7-yl]-1,3,4-oxadiazol-2-yl]-2-methyl-propanenitrile
(3R)-3-amino-1,1-dioxo-7-[5-(2,2,2-trifluoro-1,1-dimethyl-ethyl)-1,3,4-oxadiazol-2-yl]-5-[[4-[5-(trifluoromethoxy)-2-pyridyl]phenyl]methyl]-2,3-dihydro-1λ⁶,5-benzothiazepin-4-one
2-[5-[(3R)-3-amino-1,1,4-trioxo-5-[[4-[5-(trifluoromethoxy)pyrimidin-2-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(1,1,2,2,2-pentafluoroethoxy)-2-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[4-(trifluoromethoxy)pyrazol-1-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-2-ethyl-butanenitrile
2-[5-[(3R)-3-amino-5-[[4-[2-methyl-5-(trifluoromethoxy)pyrazol-3-yl]phenyl]methyl]-1,1,4-trioxo-2,3-dihydro-1λ⁶,5-benzothiazepin-7-yl]-1,3,4-oxadiazol-2-yl]-2-methyl-propanenitrile
In another embodiment, there is provided a compound of formula (I) as described herein, or a pharmaceutically acceptable salt thereof, selected from:
(3R)-3-amino-7-[5-(2-cyclopropyltetrahydrofuran-2-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
2-[5-[(3R)-3-amino-5-[[4-(4-methoxyphenyl)phenyl]methyl]-1,1,4-trioxo-2,3-dihydro-1λ⁶,5-benzothiazepin-7-yl]-1,3,4-oxadiazol-2-yl]-2-methyl-propanenitrile
(3R)-3-amino-7-[5-(1-ethoxy-1,2,2,2-tetrafluoro-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one
(3R)-3-amino-1,1-dioxo-7-[5-(2,2,2-trifluoro-1,1-dimethyl-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one
2-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-8-fluoro-1,1,4-trioxo-5-[[4-[5-(trifluoromethyl)-2-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(1,1,2,2,2-pentafluoroethoxy)-2-pyridyl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[4-(trifluoromethoxy)pyrazol-1-yl]phenyl]methyl]-2,3-dihydro-1λ⁶,5-benzothiazepin-7-yl]-1,3,4-oxadiazol-2-yl]-2-methyl-propanenitrile
2-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-2-ethyl-butanenitrile
2-[5-[(3R)-3-amino-5-[[4-[2-methyl-5-(trifluoromethoxy)pyrazol-3-yl]phenyl]methyl]-1,1,4-trioxo-2,3-dihydro-1λ⁶,5-benzothiazepin-7-yl]-1,3,4-oxadiazol-2-yl]-2-methyl-propanenitrile.

### Processes of Manufacturing

Processes for the manufacture of compounds of formula (I), or pharmaceutically acceptable salts thereof, as described herein are also an object of the invention.

The present invention provides a process for the preparation of a compound of formula (I) as described above, or a pharmaceutically acceptable salt thereof, comprising reacting of compound of formula (IX)

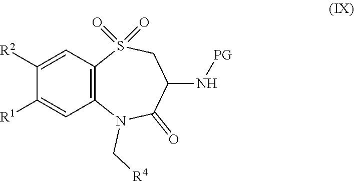

wherein R¹, R² and R⁴ are as defined herein and PG is an amino protecting group, with a suitable deprotection agent to form said compound of formula (I).

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

The present compounds of formula (I), or their pharmaceutically acceptable salts, may be prepared by a process described below (Scheme 1), together with synthetic methods known in the art of organic chemistry, or modifications and derivatizations that are familiar to those of ordinary skilled in the art.

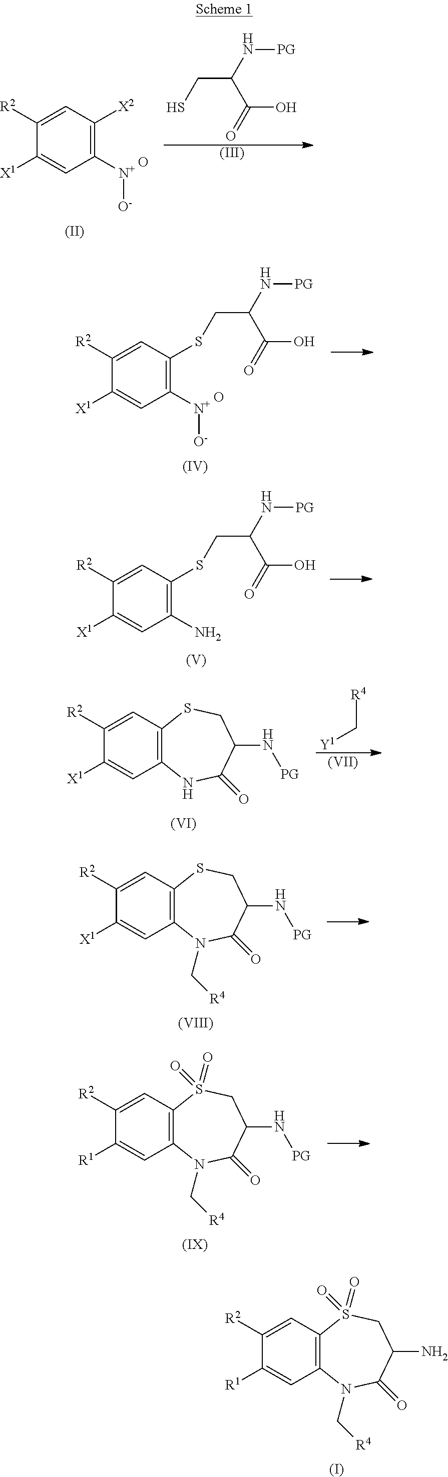

Suitable starting materials for the preparation of compounds of formula (I) are nitro compounds of formula (II) wherein X² is F or Cl and X¹ is either already R¹ or a group such as Br, CN or —CO₂Alkyl which can later be elaborated into R¹. Compounds of formula (II) can be reacted with suitably protected cysteine derivatives (III) in the presence of a base such as DIPEA at elevated temperatures in a solvent such as 1,2-dichloroethane to obtain compounds of formula (IV). The preferred protecting group (PG) of the cysteine derivative (III) is Boc. The nitro group in formula (IV) compounds can be reduced using iron in the presence of either hydrogen chloride or ammonium chloride at elevated temperatures in a solvent mixture of water and ethanol to obtain compounds of formula (V). Alternatively, this conversion can be achieved by catalytic hydrogenation. Compounds of formula (V) can be cyclized to compounds of formula (VI) using standard amide coupling conditions. Preferably, this cyclization is conducted using 2,4,6-tripropyl-1,3,5,2,4,6-trioxatriphosphinane 2,4,6-trioxide (50% solution in EtOAc) and employing a base such as DIPEA in a solvent such as DMF at room temperature. Reaction of formula (VI) compounds with compounds of formula (VII) wherein Y¹ is Cl, Br, I or a sulfonate group in the presence of a base such as potassium carbonate and if necessary with an additive such as potassium iodide in a solvent such as DMSO or DMF at room temperature affords compounds of formula (VIII). Alternatively, compounds of formula (VI) can be reacted with compounds of formula (VII) wherein Y¹ is OH in the presence of PPh₃ with an additive such as DIAD in a solvent such as toluene at elevated temperature to afford compounds of formula (VIII). For compounds of formula (VIII) wherein X¹ is Br, CN or —CO₂Alkyl, these groups can be elaborated into substituents R¹ at this stage as described in the schemes below. Compounds of formula (VIII) can then be converted into compounds of formula (IX) by reaction with an appropriate amount of an oxidant such as m-CPBA in a solvent such as DCM at room temperature. Final deprotection provides compounds of formula (I). If the N-protecting group (PG) is Boc, typical conditions for this deprotection step include TFA in a solvent such as DCM or hexafluoroisopropanol at room temperature, hydrogen chloride in solvents such as dioxane, diethyl ether or ethyl acetate at room temperature or hexafluoroisopropanol at reflux temperature. Additionally, substituents R¹ and R⁴ might contain functional groups that could be either modified prior to the removal of the N-protecting group (PG) or that might require the use of suitable protecting groups during the synthesis. These protecting groups might be removed prior to the removal of the N-protecting group (PG) or they might be removed simultaneously using suitable methods [Peter G. M. Wuts, Greene's protective groups in organic synthesis, 5th edition, Hoboken, N.J.: Wiley-Interscience]. Alternatively, compounds of formula (I) may be prepared as illustrated in scheme 2.

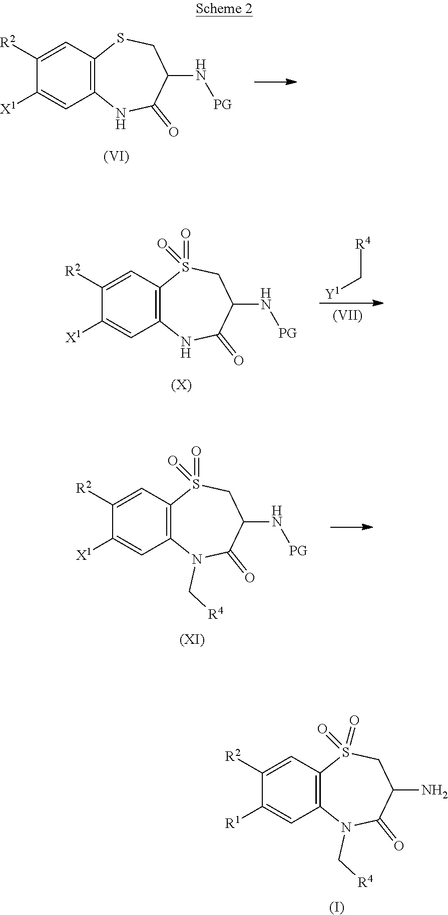

Compounds of formula (VI) can be converted into compounds of formula (X) upon reaction with an oxidant such as m-CPBA in a solvent such as DCM at room temperature. The reaction of formula (X) compounds with compounds of formula (VII) to afford compounds of formula (XI) and the subsequent conversion into compounds of formula (I) can be achieved using reaction conditions as described for the similar steps in scheme 1. If X¹ is Br, CN or —CO₂Alkyl, these groups can be elaborated into substituents R¹ at any stage of the synthesis (for compounds of formula (VI), (X) or (XI)) using methods as described for the schemes below).

Compounds of formula (I) wherein the 5-membered heteroaryl R¹ is a 1,3,4-oxadiazolyl group may be prepared as illustrated in scheme 3.

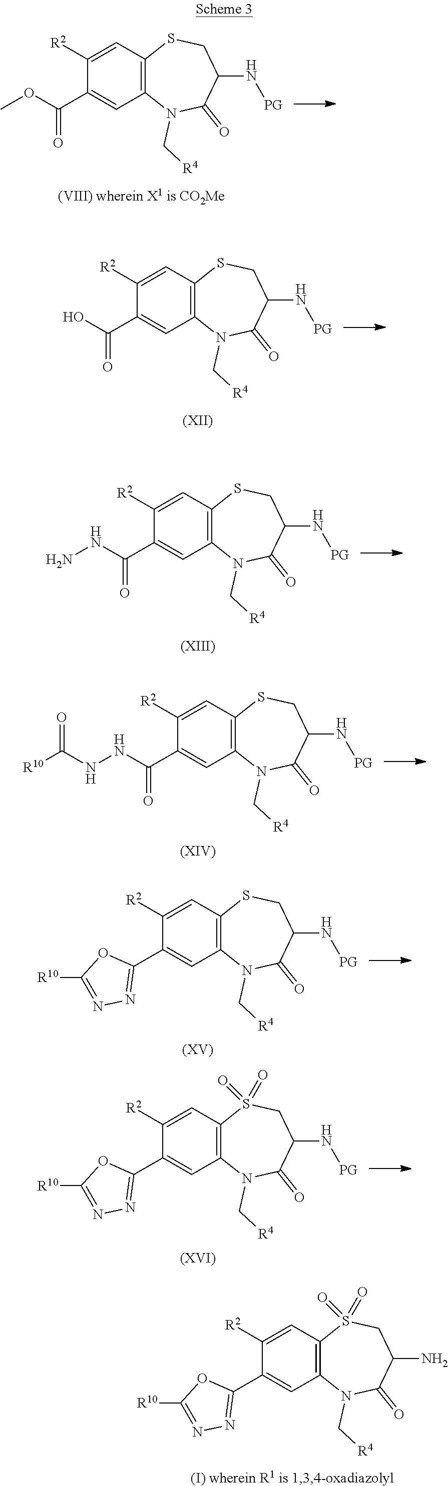

Compounds of formula (VIII) wherein X¹ is CO₂Me can be converted into compounds of formula (XII) by reaction with alkali hydroxides such as LiOH, NaOH or KOH in a mixture of solvents such as MeOH, THF and water at room temperature. Compounds of formula (XII) can be reacted with hydrazine hydrate after activation with suitable reagents such as CDI in a solvent such as THF at room temperature to obtain compounds of formula (XIII). Compounds of formula (XIII) can be reacted with carboxylic acids R¹⁰CO₂H using standard amide coupling conditions such as HATU in the presence of a base such as DIPEA in a solvent such as THF at room temperature. The coupling products of formula (XIV) can be cyclized to compounds of formula (XV) using a dehydrating reagent such as Burgess reagent or can be reacted with tosyl chloride in the presence of a base such as DIPEA at room temperature. The conversion of compounds of formula (XV) into compounds of formula (XVI) and the subsequent conversion into compounds of formula (I) can be achieved using reaction conditions as described for the similar steps in scheme 1.

Compounds of formula (I) wherein the 5-membered heteroaryl R¹ is a 1,3,4-oxadiazolyl group, R¹⁰ is N(R^{10e}R^{10f}) may be prepared as illustrated in scheme 4.

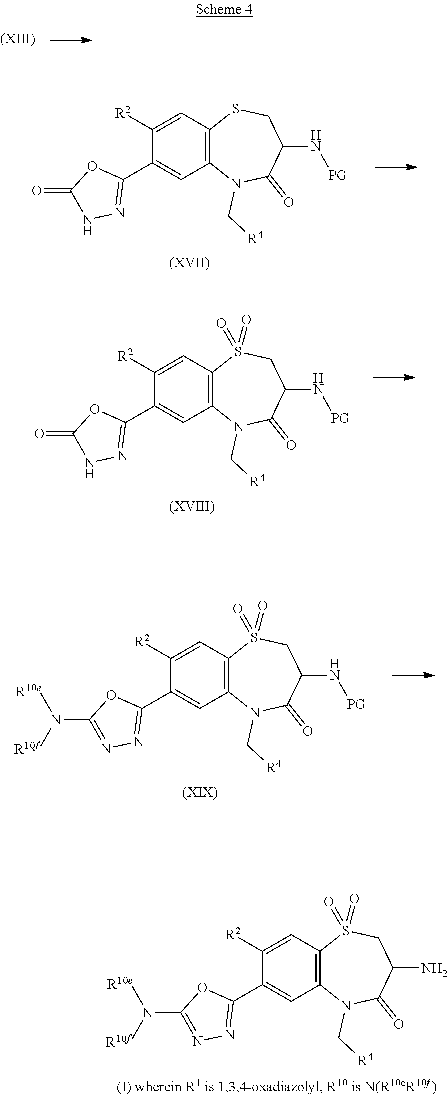

Compounds of formula (XIII) can be reacted with CDI in TIFF in the presence of a base such as TEA at room temperature to obtain compounds of formula (XVII). Oxidation of compounds of formula (XVII) with an oxidation agent such as m-CPBA in a solvent such as DCM at room temperature provides compounds of formula (XVIII). Compounds of formula (XVIII) can be transformed into compounds of formula (XIX) by reaction with amines HN(R^{10e}R^{10f}) in presence of DIPEA and BOP or PyBroP in a solvent such as DMF or dioxane at room temperature or at elevated temperatures [Org. Lett., 2008, Vol. 10, 1755-1758]. Cleavage of the N-protecting group (PG) affords compounds of formula (I).

Compounds of formula (I) wherein the 5-membered heteroaryl R¹ is a 1,2,4-oxadiazolyl group may be prepared as illustrated in scheme 5.

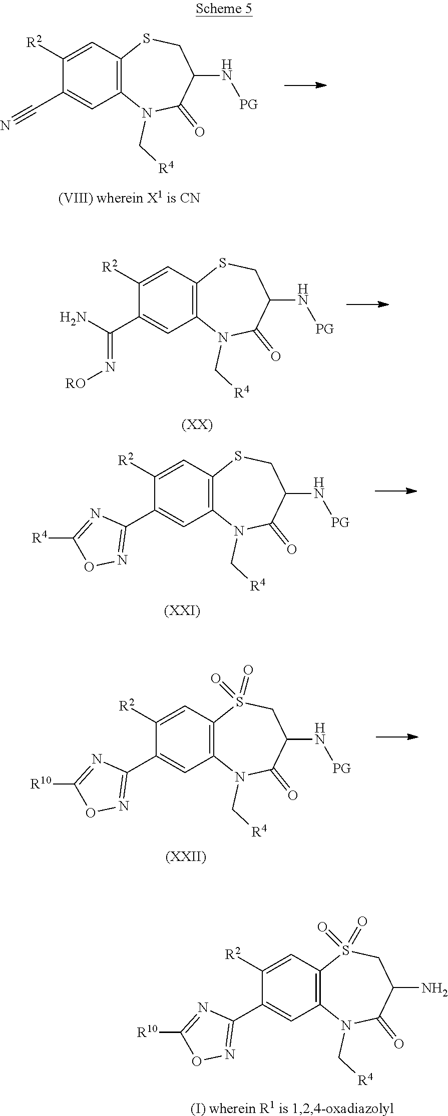

Compounds of formula (VIII) wherein X¹ is CN can be reacted with hydroxylamine hydrochloride in presence of a base such as potassium carbonate in a solvent such as ethanol at elevated temperatures to obtain amidoxime compounds of formula (XX wherein R is H). Reaction of compounds of formula (XX wherein R is H) with carboxylic acids R¹⁰CO₂H using standard amide coupling conditions such as CDI, HATU or EDCI and HOBt in the presence of a base such as DIPEA in a solvent such as acetonitrile, DMF or THF provides coupling intermediates (XX wherein R is —C(O)R¹⁰) which upon heating cyclize to the corresponding compounds of formula (XXI). Alternatively, coupling intermediates (XX wherein R is —C(O)R¹⁰) can be isolated and the cyclization step can be conducted either by heating in a solvent such as toluene or reaction with TBAOH in a solvent such as THF. The conversion of compounds of formula (XXI) into compounds of formula (XXII) and the subsequent conversion into compounds of formula (I) can be achieved using reaction conditions as described for the similar steps in scheme 1.

Compounds of formula (I) wherein the 5-membered heteroaryl R¹ is a 1,2,4-oxadiazolyl group, R¹⁰ is N(R^{10e}R^{10f}) may be prepared as illustrated in scheme 6.

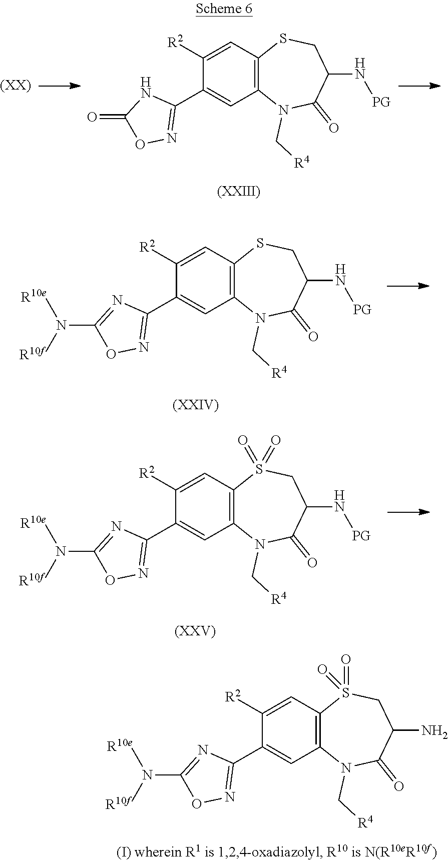

Compounds of formula (XX) can be reacted with CDI in THF in the presence of a base such as TEA at room temperature or elevated temperature to obtain compounds of formula (XXIII). Compounds of formula (XXIII) can be transformed into compounds of formula (XXIV) by reaction with amines HN(R^{10e}R^{10f}) in presence of DIPEA and PyBroP in a solvent such as dioxane at elevated temperatures. Oxidation of compounds of formula (XXIV) with an oxidation agent such as m-CPBA in a solvent such as DCM at room temperature provides compounds of formula (XXV). Cleavage of the N-protecting group (PG) affords compounds of formula (I).

Alternatively, compounds of formula (I) wherein the 5-membered heteroaryl R¹ is a 1,2,4-oxadiazolyl group, R⁴ is N(R^{4b}R^{4c}) may be prepared as illustrated in scheme 7.

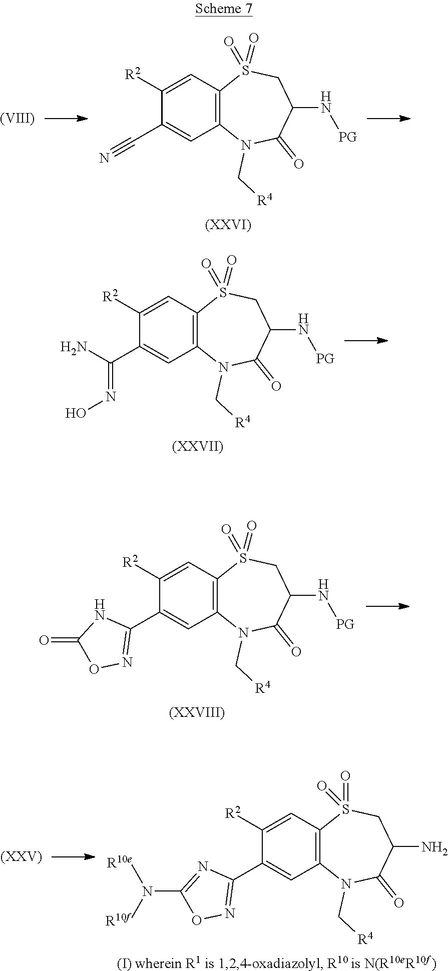

Compounds of formula (VIII) wherein X¹ is CN can be reacted with an oxidation agent such as m-CPBA in a solvent such as DCM at room temperature to provide compounds of formula (XXVI). Compounds of formula (XXVI) can be converted into compounds of formula (XXVII) by reaction with hydroxylamine hydrochloride in presence of a base such as sodium bicarbonate in a solvent such as methanol at elevated temperatures. The subsequent conversion of formula (XXVII) compounds into compounds of formula (I) can be achieved using reaction conditions as described for the similar steps in scheme 6.

### Pharmaceutical Compositions and Administration

Another object of the present invention is a pharmaceutical composition comprising a compound of formula (I) as described herein, or a pharmaceutically acceptable salt thereof, and a pharmaceutically acceptable excipient.

The compounds of formula (I) and their pharmaceutically acceptable salts can be used as medicaments, in the form of pharmaceutical preparations. The pharmaceutical preparations can be administered internally, such as orally (e.g. in the form of tablets, coated tablets, dragées, hard and soft gelatine capsules, solutions, emulsions or suspensions), nasally (e.g. in the form of nasal sprays) or rectally (e.g. in the form of suppositories). However, the administration can also be effected parenterally, such as intramuscularly or intravenously (e.g. in the form of injection solutions). The administration can also be effected topically, e.g. transdermal administration, or in form of eye drops or ear drops.

The compounds of formula (I) and their pharmaceutically acceptable salts can be processed with pharmaceutically inert, inorganic or organic carriers for the production of pharmaceutical preparations, such as tablets, coated tablets, dragées, hard gelatin capsules, injection solutions or topical formulations. Lactose, corn starch or derivatives thereof, talc, stearic acids or salts thereof, and the like can be used, for example, as such carriers for tablets, coated tablets, dragées and hard gelatin capsules.

Suitable carriers for soft gelatin capsules are, for example, vegetable oils, waxes, fats, semi-solid substances and liquid polyols and the like. Depending on the nature of the active substance no carriers are, however, usually required in the case of soft gelatin capsules.

Suitable carriers for the production of solutions and syrups are, for example, water, alcohols, polyols, saccharose, glucose, invert sugar, vegetable oil, etc.

Suitable carriers for injection solutions are, for example, water, alcohols, polyols, glycerol, vegetable oils, etc.

Suitable carriers for suppositories are, for example, natural or hardened oils, waxes, fats, semi-liquid or liquid polyols, etc.

Suitable carriers for topical ocular formulations are, for example, cyclodextrins, mannitol or many other carriers and excipients known in the art.

Moreover, the pharmaceutical preparations can contain preservatives, solubilizers, viscosity increasing substances, stabilizers, wetting agents, emulsifiers, sweeteners, colorants, flavorants, salts for varying the osmotic pressure, buffers, masking agents or antioxidants. They can also contain other therapeutically valuable substances.

Medicaments containing a compound of formula (I) or a pharmaceutically acceptable salt thereof and a pharmaceutically acceptable excipient are also an object of the present invention, as is a process for their production, which comprises bringing one or more compounds of formula (I) and/or pharmaceutically acceptable salts thereof and, if desired, one or more other therapeutically valuable substances into a galenical administration form together with one or more pharmaceutically acceptable excipients.

The dosage can vary within wide limits and will, of course, have to be adjusted to the individual requirements in each particular case. In general, in the case of oral administration a daily dosage of about 0.1 mg to 20 mg per kg body weight, preferably 0.5 mg to 4 mg per kg body weight (e.g. about 300 mg per person), divided into preferably 1-3 individual doses, which can consist, for example, of the same amounts, should be appropriate. In the case of topical administration, the formulation can contain 0.001% to 15% by weight of medicament and the required dose, which can be between 0.1 and 25 mg, and can be administered either by single dose per day or per week, or by multiple doses (2 to 4) per day, or by multiple doses per week. It will, however, be clear that the upper or lower limit given herein can be exceeded when this is shown to be indicated.

The pharmaceutical composition according to the invention may be prepared as follows.

### Preparation of Pharmaceutical Compositions Comprising Compounds of the Invention

TABLE t
Formulation (Wet Granulation)

mg/tablet

| Ingredient | 5 | 25 | 100 | 500 |
|---|---|---|---|---|
| 1) Compound of formula (I) | 5 | 25 | 100 | 500 |
| 2) Lactose Anhydrous DTG | 125 | 105 | 30 | 150 |
| 3) Sta-Rx 1500 | 6 | 6 | 6 | 30 |
| 4) Microcrystalline Cellulose | 30 | 30 | 30 | 150 |
| 5) Magnesium Stearate | 1 | 1 | 1 | 1 |
| Total | 167 | 167 | 167 | 831 |

### Manufacturing Procedure:

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

Capsule Formulation

mg/capsule

| Ingredient | 5 | 25 | 100 | 500 |
|---|---|---|---|---|
| 1) Compound of formula (I) | 5 | 25 | 100 | 500 |
| 2) Hydrous Lactose | 159 | 123 | 148 | — |
| 3) Corn Starch | 25 | 35 | 40 | 70 |
| 4) Talc | 10 | 15 | 10 | 25 |
| 5) Magnesium Stearate | 1 | 2 | 2 | 5 |
| Total | 200 | 200 | 300 | 600 |

### Manufacturing Procedure:

1. Mix ingredients 1, 2 and 3 in a suitable mixer for 30 minutes.
2. Add ingredients 4 and 5 and mix for 3 minutes.
3. Fill into a suitable capsule.

Injection Solutions

| Ingredient | mg/injection solution |
|---|---|
| Compound of formula I | 3 |
| Polyethylene Glycol 400 | 150 |
| acetic acid | q.s. ad pH 5.0 |
| water for injection solutions | ad 1.0 ml |

### Manufacturing Procedure:

A compound of formula (I) is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Indications

The compounds of formula (I) can be used in an effective amount to treat a subject, in particular a human, affected by cancer.

In one aspect, the present invention provides a compound of formula (I) described herein, or a pharmaceutically acceptable thereof, for use as a therapeutically active substance.

In a further aspect, the present invention provides a compound of formula (I) as described herein, or a pharmaceutically acceptable thereof, for use in the treatment, prevention and/or delay of progression of cancer.

In a further aspect, the present invention provides the use of a compound of formula (I) as described herein, or a pharmaceutically acceptable salt thereof, for the treatment, prevention and/or delay of progression of cancer.

In a further aspect, the present invention provides the use of a compound of formula (I) as described herein, or a pharmaceutically acceptable salt thereof, for the preparation of a medicament for the treatment, prevention and/or delay of progression of cancer.

In a further aspect, the present invention provides a method for the treatment, prevention and/or delay of progression of cancer, which method comprises administering a therapeutically effective amount of a compound of formula (I) as described herein, or a pharmaceutically acceptable salt thereof.

By the term “treatment” or “treating” and grammatical variations thereof as used herein, is meant therapeutic therapy. In reference to a particular condition, treating means: (1) to ameliorate the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy using the methods and/or compositions of the invention is also contemplated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As immunotherapeutic agents acting on immune cells rather than directly acting on the cancer cells, the present disclosure could also be foreseen for the use as anti-cancer vaccines. This also comprises approaches in which immune cells are cultured and manipulated ex vivo and the herein disclosed molecules are used as a way of conferring co-stimulation of the ex vivo manipulated cells.

In one embodiment, the cancer is a hematologic cancer such as lymphoma, a leukemia or a myeloma. A hematologic cancer contemplated herein includes, but is not limited to, one or more leukemias such as B-cell acute lymphoid leukemia (“BALL”), T-cell acute lymphoid leukemia (“TALL”), acute lymphoid leukemia (ALL); one or more chronic leukemias including but not limited to chronic myelogenous leukemia (CML) and chronic lymphocytic leukemia (CLL); additional hematologic cancers or hematologic conditions including, but not limited to B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, mucosa-associated lymphoid tissue (MALT) lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia,” which are a diverse collection of hematological conditions united by ineffective production (or dysplasia) of myeloid blood cells.

In a further embodiment, the cancer is a non-hematologic cancer such as a sarcoma, a carcinoma, or a melanoma. A non-hematologic cancer contemplated herein includes, but is not limited to, a neuroblastoma, renal cell carcinoma, colon cancer, colorectal cancer, breast cancer, epithelial squamous cell cancer, melanoma, stomach cancer, brain cancer, lung cancer (e.g. non-small cell lung cancer—NSCLC), pancreatic cancer, cervical cancer, ovarian cancer, liver cancer, bladder cancer, prostate cancer, testicular cancer, thyroid cancer, uterine cancer, adrenal cancer and head and neck cancer.

### Co-Administration of Compounds of Formula (I) and Other Agents

The compounds of formula (I) or salts thereof or a compound disclosed herein or a pharmaceutically acceptable salt thereof may be employed alone or in combination with other agents for treatment. For example, the second agent of the pharmaceutical combination formulation or dosing regimen may have complementary activities to the compound of formula (I) such that they do not adversely affect each other. The compounds may be administered together in a unitary pharmaceutical composition or separately. In one embodiment a compound or a pharmaceutically acceptable salt can be co-administered with a cytotoxic agent to treat proliferative diseases and cancer.

The term “co-administering” refers to either simultaneous administration, or any manner of separate sequential administration, of a compound of formula (I) or a salt thereof or a compound disclosed herein or a pharmaceutically acceptable salt thereof and a further active pharmaceutical ingredient or ingredients, including cytotoxic agents and radiation treatment. If the administration is not simultaneous, the compounds are administered in a close time proximity to each other. Furthermore, it does not matter if the compounds are administered in the same dosage form, e.g. one compound may be administered topically and another compound may be administered orally.

Typically, any agent that has anti-cancer activity may be co-administered. Examples of such agents can be found in Cancer Principles and Practice of Oncology by V. T. Devita and S. Heilman (editors), 6th edition (Feb. 15, 2001), Lippincott Williams & Wilkins Publishers. A person of ordinary skill in the art would be able to discern which combinations of agents would be useful based on the particular characteristics of the drugs and the disease involved.

In one aspect, the present invention provides a pharmaceutical composition described herein, further comprising an additional therapeutic agent.

In one embodiment, said additional therapeutic agent is a chemotherapeutic agent.

In one embodiment, said additional therapeutic agent is a cytotoxic agent.

In one embodiment, said additional therapeutic agent is an immuno-oncology agent.

The term “cytotoxic agent” as used herein refers to a substance that inhibits or prevents a cellular function and/or causes cell death or destruction. Cytotoxic agents include, but are not limited to, radioactive isotopes (At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG(geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®., Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; Eiziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin I and cryptophycin 8); adrenocorticosteroids (including prednisone and prednisolone); cyproterone acetate; 5a-reductases including finasteride and dutasteride); vorinostat, romidepsin, panobinostat, valproic acid, mocetinostat dolastatin; aldesleukin, talc duocarmycin (including the synthetic analogs, KW-2189 and CBI-TM I); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γiI and calicheamicin coll (Angew Chem. Inti. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, 111), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-I I; topoisomerase inhibitor RFS 2000; difluoromethylomithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY1 17018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase I inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idee), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgGi λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind toEGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as EL1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al, J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (Cl 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(I -phenylethyl)amino]-1 H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule FIER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-I inhibitors such as antisense agent ISIS-5132 available from ISIS Pharmaceuticals which inhibit Raf-I signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor C1-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Wamer-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI- 1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-ICl I (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (PEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFa) blockers such as etanercept (Enbrel), infliximab (Remicade), adalimumab (Humira), certolizumab pegol (Cimzia), golimumab (Simponi), Interleukin I (IL-I) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-Ml prime; Secreted homotrimeric LTa3 and membrane bound heterotrimer LTa I/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², p³², Pb²¹² and radioactive isotopes of Lu); miscellaneous investigational agents such as thioplatin, PS-341, phenylbutyrate, ET-18-OCH3, or famesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTQNEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifamib (R11577); orafenib, ABT510; Bcl-2 inhibitor such as oblimersen sodium (GENASENSE®); pixantrone; famesyltransferase inhibitors such as lonafamib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

In another embodiment, compounds of formula (I) can be co-formulated with an immuno-oncology agent. Immuno-oncology agents include, for example, a small molecule drug, antibody, or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human. In another aspect, the antibody is a bispecific antibody.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTfiR, LIGHT, DcR3, HVEM, VEGI/TL1A, TRAMP/DR3, EDAR, EDA1, XEDAR, EDA2, TNFR1, Lymphotoxin α/TNPβ, TNFR2, TNF a, LT R, Lymphotoxin a 1β2, FAS, FASL, RELT, DR6, TROY, NGFR.

In one aspect, T cell responses can be stimulated by a combination of a compound of formula (I) and one or more of (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

Other agents that can be combined with compounds of formula (I) for the treatment of cancer include antagonists of inhibitory receptors on NK cells or agonists of activating receptors on NK cells. For example, compounds of formula (I) can be combined with antagonists of KIR, such as lirilumab.

Yet other agents for combination therapies include agents that inhibit or deplete macrophages or monocytes, including but not limited to CSF-1R antagonists such as CSF-1R antagonist antibodies including RG7155 or FPA-008.

In another aspect, compounds of formula (I) can be used with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In some embodiments, the immuno-oncology agent is a CTLA-4 antagonist, such as an antagonistic CTLA-4 antibody. Suitable CTLA-4 antibodies include, for example, YERVOY (ipilimumab) or tremelimumab. In another aspect, the immuno-oncology agent is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514; WO2012/145493). The immuno-oncology agent may also include pidilizumab (CT-011), though its specificity for PD-1 binding has been questioned. Another approach to target the PD-1 receptor is the recombinant protein composed of the extracellular domain of PD-L2 (B7-DC) fused to the Fc portion of IgG1, called AMP-224 In another aspect, the immuno-oncology agent is a PD-L1 antagonist, such as an antagonistic PD-L1 antibody. Suitable PD-L1 antibodies include, for example, TECENTRIQ (atezolizumab) (RG7446; WO2010/077634), durvalumab (MEDI4736), BMS-936559 (WO2007/005874), and MSB0010718C (WO2013/79174).

In another aspect, the immuno-oncology agent is a LAG-3 antagonist, such as an antagonistic LAG-3 antibody. Suitable LAG3 antibodies include, for example, BMS-986016 (WO2010/19570, WO2014/08218), or IMP-731 or IMP-321 (WO2008/132601, WO2009/44273).

In another aspect, the immuno-oncology agent is a CD137 (4-1BB) agonist, such as an agonistic CD137 antibody. Suitable CD137 antibodies include, for example, urelumab and PF-05082566 (WO2012/32433).

In another aspect, the immuno-oncology agent is a GITR agonist, such as an agonistic GITR antibody. Suitable GITR antibodies include, for example, BMS-986153, BMS-986156, TRX-518 (WO2006/105021, WO2009/009116) and MK-4166 (WO2011/028683).

In another aspect, the immuno-oncology agent is an IDO antagonist. Suitable IDO antagonists include, for example, INCB-024360 (WO2006/122150, WO2007/75598, WO2008/36653, WO2008/36642), indoximod, or NLG-919 (WO2009/73620, WO2009/1156652, WO2011/56652, WO2012/142237).

In another aspect, the immuno-oncology agent is an OX40 agonist, such as an agonistic OX40 antibody. Suitable OX40 antibodies include, for example, MEDI-6383 or MEDI-6469. In another aspect, the immuno-oncology agent is an OX40L antagonist, such as an antagonistic OX40 antibody. Suitable OX40L antagonists include, for example, RG-7888 (WO06/029879).

In another aspect, the immuno-oncology agent is a CD40 agonist, such as an agonistic CD40 antibody. In yet another embodiment, the immuno-oncology agent is a CD40 antagonist, such as an antagonistic CD40 antibody. Suitable CD40 antibodies include, for example, lucatumumab or dacetuzumab.

In another aspect, the immuno-oncology agent is a CD27 agonist, such as an agonistic CD27 antibody. Suitable CD27 antibodies include, for example, varlilumab.

In another aspect, the immuno-oncology agent is MGA271 (to B7H3) (WO2011/109400).

### EXAMPLES

The invention will be more fully understood by reference to the following examples. The claims should not, however, be construed as limited to the scope of the examples.

### 1) Preparative Examples

All reaction examples and intermediates were prepared under an argon atmosphere if not specified otherwise.

### 1.1) General Procedures

Alkylation: General procedure 1a

To a solution of an intermediate of formula (VI) (2.74 mmol) in DMF (10 mL) were added at RT potassium carbonate (1.14 g, 8.23 mmol), potassium iodide (228 mg, 1.37 mmol) and a reagent of formula (VII) (3.29 mmol). The reaction was stirred at RT for 2 h, quenched with water and extracted twice with DCM. The combined organic layers were washed with water, saturated aqueous sodium chloride solution, dried over sodium sulfate, filtered and the solvent was evaporated under reduced pressure. The desired product (VIII) was used crude in the next step or was purified by flash column chromatography on silica gel or by reverse phase preparative HPLC.

### Alkylation: General Procedure 1b

To a solution of an intermediate of formula (VI) (0.2 mmol) in toluene (3 ml) under inert atmosphere was added a reagent of formula (VII) (0.22 mmol), PPh₃ (0.4 mmol) and DIAD (0.4 mmol). The mixture was then heated to 50° C. for 4 h. After cooling to RT, the mixture was concentrated and diluted with EtOAc. This solution was then washed with brine (3×), dried over sodium sulfate, filtered and the solvent evaporated under reduced pressure. The desired product (VIII) was used crude in the next step or purified by flash column chromatography on silica gel or by reverse phase preparative HPLC.

### Saponification: General Procedure 2

To a solution of an intermediate of formula (VIII, wherein X¹ is CO₂Me) (4 mmol) in THF (18 ml), MeOH (3 ml) and water (6 ml) was added LiOH hydrate (8 mmol) and stirred at RT for 2 h. 1N HCl was added and the resulting suspension extracted three times with EtOAc. The combined organic layers were washed with brine and then dried over sodium sulfate, filtered and the solvent evaporated under reduced pressure. The desired product (XII) was used crude in the next step or purified by flash column chromatography on silica gel or by reverse phase preparative HPLC.

### Hydrazide Formation: General Procedure 3

To a solution of an intermediate of formula (XII) (4.5 mmol) in THF (20 ml) was added CDI (5.7 mmol) and stirred at RT for 90 min. To this solution was then added a mixture of hydrazine hydrate (13.5 mmol) in THF (3.3 ml) and stirred for 1 h. The reaction mixture was diluted with water and EtOAc. The layers were separated and the aqueous phase washed twice with EtOAc. The combined organic layers were washed with brine and then dried over sodium sulfate, filtered and the solvent evaporated under reduced pressure. The desired product (XIII) was used crude in the next step or purified by flash column chromatography on silica gel or by reverse phase preperative HPLC.

### Hydrazide Coupling: General Procedure 4a

To a solution of an intermediate of formula (XIII) (0.3 mmol) in THF (3 ml) was added a carboxylic acid of formula R¹⁰CO₂H (0.45 mmol), DIPEA (0.6 mmol) and HATU (0.45 mmol). The resulting solution was stirred for 4 h at RT. The reaction mixture was diluted with EtOAc and water. The layers were separated and the aqueous phase extracted twice with EtOAc. The combined organic layers were washed with brine and then dried over sodium sulfate, filtered and the solvent evaporated under reduced pressure. The desired product (XIV) was used crude in the next step or purified by flash column chromatography on silica gel or by reverse phase preperative HPLC.

### Hydrazide Coupling: General Procedure 4b

To a solution of an intermediate of formula (XIII) (0.5 mmol) in THF (5 ml) was added a carboxylic acid of formula R¹⁰CO₂H (0.5 mmol), DIPEA (1.5 mmol) and T₃P (50% in EtOAc, 1.5 mmol). The resulting solution was stirred at 60° C. for 2 h. The reaction was then cooled to RT and diluted with water. The mixture was extracted three times with EtOAc. The combined organic layers were washed with brine and then dried over sodium sulfate, filtered and the solvent evaporated under reduced pressure. The desired product (XIV) was used crude in the next step or purified by flash column chromatography on silica gel or by reverse phase preperative HPLC.

### 1,3,4-Oxadiazole Cyclization: General Procedure 5a

To a solution of an intermediate of formula (XIV) (0.3 mmol) in THF (3 ml) was added Burgess reagent (0.9 mmol). The resulting solution was stirred at RT overnight. Water was added and the mixture was extracted three times with EtOAc. The combined organic layers were washed with brine and then dried over sodium sulfate, filtered and the solvent evaporated under reduced pressure. The desired product (XV) was used crude in the next step or purified by flash column chromatography on silica gel or by reverse phase preperative HPLC.

### 1,3,4-Oxadiazole Cyclization: General Procedure 5b

To a solution of an intermediate of formula (XIV) (0.1 mmol) in acetonitrile (1.3 ml) was added p-toluenesulfonyl chloride (0.3 mmol) and DIPEA (0.2 mmol). The resulting solution was stirred at RT for 30 min. The reaction was diluted with water and extracted three times with EtOAc. The combined organic layers were washed with brine and then dried over sodium sulfate, filtered and the solvent evaporated under reduced pressure. The desired product (XV) was used crude in the next step or purified by flash column chromatography on silica gel or by reverse phase preperative HPLC.

### Amidoxime Formation: General Procedure 6

To a solution of an intermediate of formula (VIII, wherein X¹ is CN) (0.3 mmol) in EtOH (2.5 ml) was added solid NaHCO₃ (1.5 mmol) and hydroxylamine hydrochloride (0.6 mmol). The resulting suspension was heated to 80° C. for 90 min and then allowed to cool to RT. The suspension was filtered and the filter cake washed with EtOH and DCM. The filtrate was concentrated under reduced pressure and the remaining solid was dissolved in DCM and washed with water and brine, dried over anhydrous sodium sulfate, filtered and the solvent evaporated under reduced pressure. The desired product (XX wherein R is H) was used crude in the next step or purified by flash column chromatography on silica gel or by reverse phase preperative HPLC.

Direct 1,2,4-Oxadiazole Formation from Amidoxime: General Procedure 7a

To a solution of a carboxylic acid of formula R¹⁰CO₂H (2.4 mmol) in DMF (5 ml) was added CDI (2.64 mmol) and stirred for 60 min. Then, a solution of an intermediate of formula (XX, wherein R is H) (1.2 mmol) in DMF (5 ml) was added and the resulting mixture heated to 120° C. for 4 h. The reaction was allowed to cool RT and water and EtOAc were added. The layers were separated and the aqueous phase extracted twice with EtOAc. The combined organic layers were washed with 1N HCl, dried over anhydrous sodium sulfate, filtered and the solvent evaporated under reduced pressure. The desired product (XXI) was used crude in the next step or purified by flash column chromatography on silica gel or by reverse phase preperative HPLC.

Direct 1,2,4-Oxadiazole Formation from Amidoxime: General Procedure 7b

To a solution of of an intermediate of formula (XX, wherein R is H) (0.3 mmol) in THF (5 mL) were added a carboxylic acid of formula R¹⁰CO₂H (0.45 mmol), DIPEA (0.76 mmol) and 2,4,6-tripropyl-1,3,5,2,4,6-trioxatriphosphinane 2,4,6-trioxide 50% solution in EtOAc (0.6 mmol) and the reaction was stirred at RT for 16 h. The reaction mixture was quenched with water and extracted twice with EtOAc, washed with 1M aqueous NaOH solution, 1M aqueous HCl solution and brine, dried over sodium sulfate, filtered and concentrated in vacuo. The desired product (XXI) was used crude in the next step or was purified by flash column chromatography on silica gel or by reverse phase preparative HPLC.

Direct 1,2,4-Oxadiazole Formation from Amidoxime: General Procedure 7c

To solution of an intermediate of formula (XX, wherein R is H) (0.2 mmol) in DMF (1.5 ml) was added a carboxylic acid of formula R¹⁰CO₂H (0.24 mmol), EDC hydrochloride (0.4 mmol), DIPEA (0.6 mmol) and HOBt (0.3 mmol) and the resulting mixture was heated to 80° C. for 8 h. The reaction was allowed to cool RT and water and EtOAc were added. The layers were separated and the aqueous phase extracted twice with EtOAc. The combined organic layers were washed with brine, dried over anhydrous sodium sulfate, filtered and the solvent evaporated under reduced pressure. The desired product (XXI) was used crude in the next step or purified by flash column chromatography on silica gel or by reverse phase preperative HPLC.

Amidoxime Coupling with RCO₂H: General Procedure 8a

To a solution of an intermediate of formular (XX, wherein R is H) (1.0 mmol) in THF (8.5 ml) was added a carboxylic acid of formula R¹⁰CO₂H (0.12 mmol), DIPEA (2.0 mmol) and HATU (0.15 mmol) and the reaction was stirred at RT for 4 h. Water and EtOAc were added and the layers were separated. The aqueous phase was extracted twice with EtOAc. The combined organic layers were washed with brine, dried over anhydrous sodium sulfate, filtered and the solvent evaporated under reduced pressure. The desired product (XX, wherein R is —CO(R¹⁰)) was used crude in the next step or purified by flash column chromatography on silica gel or by reverse phase preperative HPLC.

Amidoxime Coupling with RCO₂H: General Procedure 8b

To solution of an intermediate of formula (XX, wherein R is H) (0.2 mmol) in DMF (1.5 ml) was added a carboxylic acid of formula R¹⁰CO₂H (0.24 mmol), EDC hydrochloride (0.4 mmol), DIPEA (0.6 mmol) and HOBt (0.3 mmol) and the resulting mixture was stirred at RT for 16 h. Water and EtOAc were added, the layers were separated and the aqueous phase extracted twice with EtOAc. The combined organic layers were washed with brine, dried over anhydrous sodium sulfate, filtered and the solvent evaporated under reduced pressure. The desired product (XX, wherein R is —CO(R¹⁰)) was used crude in the next step or purified by flash column chromatography on silica gel or by reverse phase preperative HPLC.

Amidoxime Coupling with RCO₂H: General procedure 8c

To a solution of a carboxylic acid of formula R¹⁰CO₂H (0.11 mmol) in acetonitrile (0.33 ml) was added CDI (0.12 mmol) and stirred at RT for 60 min. To this mixture was then added a solution of an intermediate of formula (XX, wherein R is H) (0.1 mmol) in acetonitrile (0.33 ml) and stirred for 60 min at RT. The reaction was diluted with DCM and water was added. The layers were separated and the aqueous phase extracted twice with DCM. The combined organic layers were washed with brine, dried over anhydrous sodium sulfate, filtered and the solvent evaporated under reduced pressure. The desired product (XX, wherein R is —CO(R¹⁰)) was used crude in the next step or purified by flash column chromatography on silica gel or by reverse phase preperative HPLC.

### 1,2,4-Oxadiazole Cyclization: General Procedure 9a

A solution of an intermediate of formula (XX, wherein R is —CO(R¹⁰)) (0.15 mmol) in toluene (1 ml) was heated to 120° C. for 16 h. The solvent was then evaporated under reduced pressure. The desired product (XXI) was used crude in the next step or purified by flash column chromatography on silica gel or by reverse phase preperative HPLC.

1,2,4-Oxadiazole cyclization: General procedure 9b

To a solution of an intermediate of formula (XX, wherein R is —CO(R¹⁰)) (0.12 mmol) in THF (1.2 ml) was added tetrabutylammonium hydroxide (0.06 mmol) and stirred at RT for 30 min. The reaction was diluted with EtOAc and washed with sat. aq. NaHCO₃. The aqueous phase was then washed twice with EtOAc and the combined organic layers dried over anhydrous sodium sulfate, filtered and the solvent evaporated under reduced pressure. The desired product (XXI) was used crude in the next step or purified by flash column chromatography on silica gel or by reverse phase preperative HPLC.

### Oxidation: General Procedure 10

A solution of an intermediate of formula (VIII) (2.74 mmol) and m-CPBA (1.18 g, 6.85 mmol) in DCM (10 mL) was stirred at RT for 1 day. The reaction was diluted with EtOAc and THF, washed with 2N aqueous sodium hydroxide solution, 1N aqueous HCl solution and saturated aqueous sodium chloride solution, dried over sodium sulfate, filtered and the solvent was removed under reduced pressure. The desired product (IX) was used crude in the next step or was purified by flash column chromatography on silica gel or by reverse phase preparative HPLC.

### Boc De-Protection: General Procedure 11a

To a solution of an intermediate of formula (IX) (0.250 mmol) in 1,1,1,3,3,3-hexafluoropropan-2-ol (4 mL) was added HCl/dioxane or HCl/Et₂O (0.5 mmol, 2 eq) at 0° C. The reaction mixture was stirred at 20° C. for 2 h. The solvent was evaporated and the resulting solid taken up in DCM and concentrated again to remove trace 1,1,1,3,3,3-hexafluoropropan-2-ol. This process was repeated two times followed by drying under high vacuum to obtain the desired product (I).

### Boc De-Protection: General Procedure 11b

A solution of an intermediate of formula (IX) (22.7 μmol) in 1,1,1,3,3,3-hexafluoropropan-2-ol (1.5 mL) was stirred at reflux for 5 days. The solvent was evaporated and the remaining residue was dried under high vacuum to yield desired product (I).

### Boc De-Protection: General Procedure 11c

To a solution of an intermediate of formula (IX) (0.250 mmol) in EtOAc (4 mL) was added HCl/EtOAc (4.0 mL, 16 mmol, 63 eq) at 0° C. The reaction mixture was stirred at 20° C. for 3 h and then concentrated in vacuo. The remaining residue was purified by prep-HPLC and dried by lyophilization to obtain the desired product (I).

### Bromination: General Procedure 12

A solution of an intermediate of formula (VII, wherein Y¹ is H) (1.34 mmol) in acetonitrile (5.3 mL) was added N-bromosuccinimide (1.6 mmol) and 2,2′-azobis(2-methylpropionitrile) (0.13 mmol) and stirred at 80° C. for 3 h. The reaction was quenched upon addition of sat. aqueous sodium thiosulfate and EtOAc and the mixture was stirred vigorously for 5 min and the phases separated. The aqueous phase was extracted twice with EtOAc. The combined organic phase was dried over sodium sulfate, filtered and concentrated under reduced pressure. The desired product (VII, wherein Y¹ is Br) was used crude in the next step or purified by flash column chromatography on silica gel.

### Amination: General Procedure 13

To a solution of an intermediate of formula (XVIII) (0.06 mmol) in 1,4-dioxane (0.6 ml) was added an amine of formula HN(R¹⁰ eR¹⁰f) (0.12 mmol), DIPEA (0.18 mmol) and PyBroP (0.072 mmol). The mixture was heated to 50° C. for 90 min. After cooling to RT, EtOAc and water were added and the reaction stirred vigorously for 5 min. The layers were separated and the aqueous phase extracted twice with EtOAc. The combined organic layers were dried over anhydrous sodium sulfate, filtered and concentrated in vacuo. The desired product (XIX) was used crude in the next step or purified by flash column chromatography on silica gel or by reverse phase preparative HPLC.

### Example 1

### (3R)-3-amino-7-(5-tert-butyl-1,3,4-oxadiazol-2-yl)-8-fluoro-5-[[4-(1-methylpyrazol-3-yl)phenyl]methyl]-1,1-dioxo-2,3-dihydro-1λ⁶,5-benzothiazepin-4-one

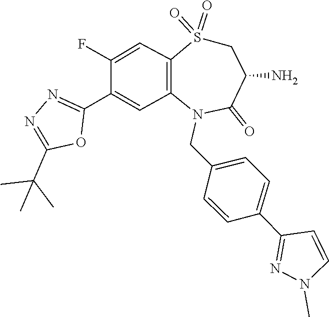 [SMILES: Cn1ccc(-c2ccc(CN3C(=O)[C@@H](N)CS(=O)(=O)c4cc(F)c(-c5nnc(C(C)(C)C)o5)cc43)cc2)n1]

### Step a) (3R)-3-(tert-butoxycarbonylamino)-8-fluoro-4-oxo-3,5-dihydro-2H-1,5-benzothiazepine-7-carboxylic Acid

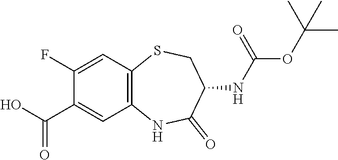 [SMILES: CC(C)(C)OC(=O)N[C@H]1CSc2cc(F)c(C(=O)O)cc2NC1=O]

The title compound was prepared from methyl (3R)-3-(tert-butoxycarbonylamino)-8-fluoro-4-oxo-3,5-dihydro-2H-1,5-benzothiazepine-7-carboxylate (9.5 g, 25.65 mmol, CAS 202449-38-7) in analogy to general procedure 2 and was obtained as brown solid (9.5 g, 26.66 mmol, 104% yield). MS (ESI): 301.0 [M-isobutene+H]⁺

### Step b) tert-butyl N-[(3R)-8-fluoro-7-(hydrazinecarbonyl)-4-oxo-3,5-dihydro-2H-1,5-benzothiazepin-3-yl]carbamate

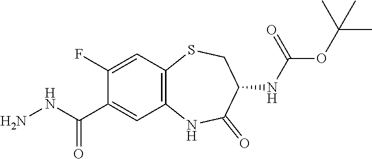 [SMILES: CC(C)(C)OC(=O)N[C@H]1CSc2cc(F)c(C(=O)NN)cc2NC1=O]

The title compound was prepared from (3R)-3-(tert-butoxycarbonylamino)-8-fluoro-4-oxo-3,5-dihydro-2H-1,5-benzothiazepine-7-carboxylic acid (1300 mg, 2.81 mmol) in analogy to general procedure 3 and was obtained as light brown oil (1300 mg, 3.51 mmol, 95% yield). MS (ESI): 315.2 [M-isobutene+H]⁺

### Step c) tert-butyl N-[(3R)-7-[(2,2-dimethylpropanoylamino)carbamoyl]-8-fluoro-4-oxo-3,5-dihydro-2H-1,5-benzothiazepin-3-yl]carbamate

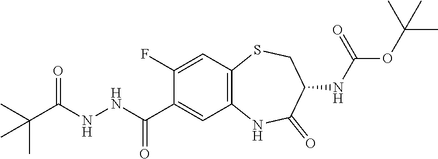 [SMILES: CC(C)(C)OC(=O)N[C@H]1CSc2cc(F)c(C(=O)NNC(=O)C(C)(C)C)cc2NC1=O]

The title compound was prepared from tert-butyl N-[(3R)-8-fluoro-7-(hydrazinecarbonyl)-4-oxo-3,5-dihydro-2H-1,5-benzothiazepin-3-yl]carbamate (1287 mg, 2.66 mmol, 1 eq) and pivalic acid (0.31 mL, 2.68 mmol, 1.01 eq) in analogy to general procedure 4a and was obtained as yellow solid (1240 mg, 2.73 mmol, 97% yield). MS (ESI): 399.1 [M-isobutene+H]⁺

### Step d) tert-butyl N-[(3R)-7-(5-tert-butyl-1,3,4-oxadiazol-2-yl)-8-fluoro-4-oxo-3,5-dihydro-2H-1,5-benzothiazepin-3-yl]carbamate

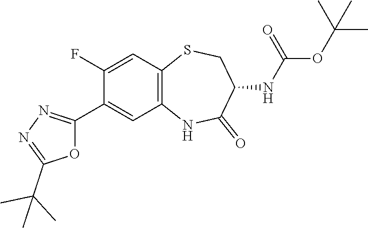 [SMILES: CC(C)(C)OC(=O)N[C@H]1CSc2cc(F)c(-c3nnc(C(C)(C)C)o3)cc2NC1=O]

The title compound was prepared from tert-butyl N-[(3R)-7-[(2,2-dimethylpropanoylamino)carbamoyl]-8-fluoro-4-oxo-3,5-dihydro-2H-1,5-benzothiazepin-3-yl]carbamate (2.69 g, 5.92 mmol) in analogy to general procedure 5a and was obtained as light yellow solid (2.57 g, 5.36 mmol, 91% yield). MS (ESI): 381.1 [M-isobutene+H]⁺

### Step e) tert-butyl N-[(3R)-7-(5-tert-butyl-1,3,4-oxadiazol-2-yl)-8-fluoro-1,1,4-trioxo-3,5-dihydro-2H-1λ⁶,5-benzothiazepin-3-yl]carbamate

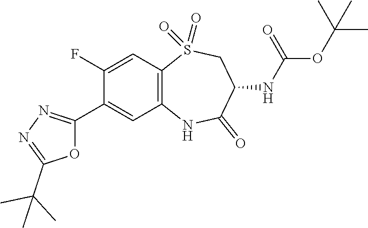 [SMILES: CC(C)(C)OC(=O)N[C@H]1CS(=O)(=O)c2cc(F)c(-c3nnc(C(C)(C)C)o3)cc2NC1=O]

The title compound was prepared from tert-butyl N-[(3R)-7-(5-tert-butyl-1,3,4-oxadiazol-2-yl)-8-fluoro-4-oxo-3,5-dihydro-2H-1,5-benzothiazepin-3-yl]carbamate (800 mg, 1.83 mmol) in analogy to general procedure 10 and was obtained as light yellow solid (830 mg, 1.77 mmol, 97% yield). MS (ESI): 413.1 [M-isobutene+H]⁺

### Step f) tert-butyl N-[(3R)-7-(5-tert-butyl-1,3,4-oxadiazol-2-yl)-8-fluoro-5-[[4-(1-methylpyrazol-3-yl)phenyl]methyl]-1,1,4-trioxo-2,3-dihydro-1λ⁶, 5-benzothiazepin-3-yl]carbamate

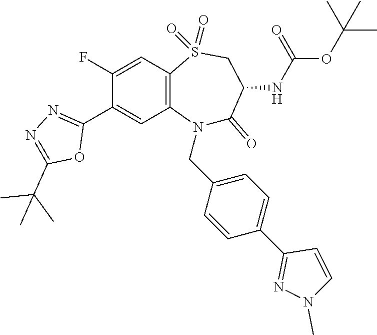 [SMILES: Cn1ccc(-c2ccc(CN3C(=O)[C@@H](NC(=O)OC(C)(C)C)CS(=O)(=O)c4cc(F)c(-c5nnc(C(C)(C)C)o5)cc43)cc2)n1]

The title compound was prepared from tert-butyl N-[(3R)-7-(5-tert-butyl-1,3,4-oxadiazol-2-yl)-8-fluoro-1,1,4-trioxo-3,5-dihydro-2H-1λ⁶,5-benzothiazepin-3-yl]carbamate (100 mg, 0.21 mmol, 1.0 eq) and 4-(1-methyl-1H-pyrazolo-3-yl)benzene methanol (44 mg, 0.23 mmol, 1.1 eq, CAS 179055-20-0) in analogy to general procedure 1b and was obtained as white solid (180 mg, 0.28 mmol, 62% yield). MS (ESI): 639.2 [M+H]⁺

### Step g) (3R)-3-amino-7-(5-tert-butyl-1,3,4-oxadiazol-2-yl)-8-fluoro-5-[[4-(1-methylpyrazol-3-yl)phenyl]methyl]-1,1-dioxo-2,3-dihydro-1λ⁶, 5-benzothiazepin-4-one

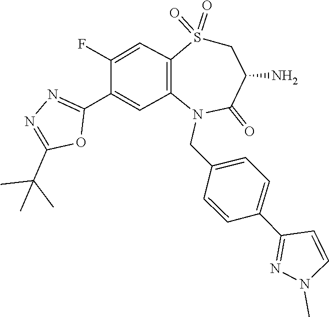 [SMILES: Cn1ccc(-c2ccc(CN3C(=O)[C@@H](N)CS(=O)(=O)c4cc(F)c(-c5nnc(C(C)(C)C)o5)cc43)cc2)n1]

The title compound was prepared from tert-butyl N-[(3R)-7-(5-tert-butyl-1,3,4-oxadiazol-2-yl)-8-fluoro-5-[[4-(1-methylpyrazol-3-yl)phenyl]methyl]-1,1,4-trioxo-2,3-dihydro-1λ⁶,5-benzothiazepin-3-yl]carbamate (180 mg, 0.28 mmol) in analogy to general procedure 11c and was obtained as white solid (15.5 mg, 0.03 mmol, 10% yield). MS (ESI): 539.2 [M+H]⁺

The example of the following table were prepared in analogy to Example 1, using the benzyl alcohol building block.

| | | | | MS, ESI: |
|---|---|---|---|---|
| Ex. | Structure | Systematic Name | Building Block | m/z |
| 2 | 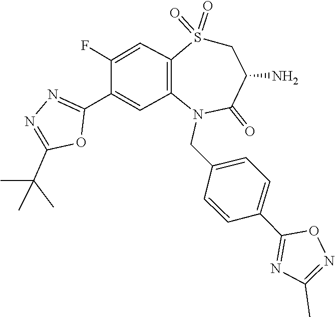 [SMILES: Cc1noc(-c2ccc(CN3C(=O)[C@@H](N)CS(=O)(=O)c4cc(F)c(-c5nnc(C(C)(C)C)o5)cc43)cc2)n1] | (3R)-3-amino-7-(5- tert-butyl-1,3,4- oxadiazol-2-yl)-8- fluoro-5-[[4-(3- methyl-1,2,4- oxadiazol-5- yl)phenyl]methyl]- 1,1-dioxo-2,3- dihydro-1λ⁶,5- benzothiazepin-4- one (*) | 4-(3-methyl- 1,2,4- oxadiazol-5- yl)benzene metnahol (CAS 362529-02-0) | 541.2 [M + H]⁺ |

* as hydrochloride salt

### Example 88

### 2-[5-[(3R)-3-amino-8-fluoro-1,1,4-trioxo-5-[[4-[5-(trifluoromethyl)-2-pyridyl]phenyl]methyl]-2,3-dihydro-1λ⁶,5-benzothiazepin-7-yl]-1,3,4-oxadiazol-2-yl]-2-methyl-propanenitrile

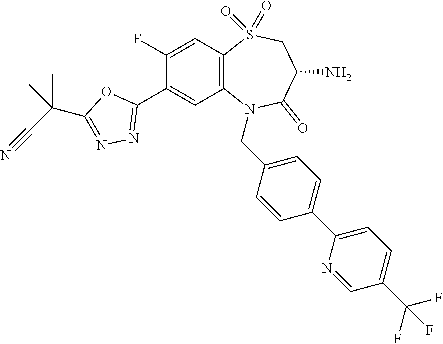 [SMILES: CC(C)(C#N)c1nnc(-c2cc3c(cc2F)S(=O)(=O)C[C@H](N)C(=O)N3Cc2ccc(-c3ccc(C(F)(F)F)cn3)cc2)o1]

### Step a) tert-butyl N-[(3R)-7-[[(2-cyano-2-methyl-propanoyl)amino]carbamoyl]-8-fluoro-4-oxo-3,5-dihydro-2H-1,5-benzothiazepin-3-yl]carbamate

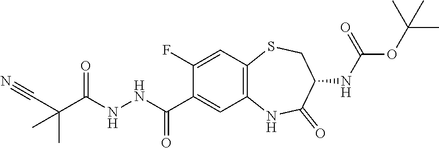 [SMILES: CC(C)(C)OC(=O)N[C@H]1CSc2cc(F)c(C(=O)NNC(=O)C(C)(C)C#N)cc2NC1=O]

To a suspension of tert-butyl N-[(3R)-8-fluoro-7-(hydrazinecarbonyl)-4-oxo-3,5-dihydro-2H-1,5-benzothiazepin-3-yl]carbamate (150 mg, 404.97 μmol, 1.0 eq, Example 1, step b) and 2-cyano-2-methyl-propionic acid (55 mg, 485.96 μmol, 1.2 eq) in THF (2.85 ml) was added HATU (231 mg, 607.45 μmol, 1.5 eq) and DIPEA (130.9 mg, 176.8 uL, 1.01 mmol, 2.5 eq) at room temperature and the mixture was stirred for 4 h. The mixture was partitioned between EtOAc and water. The aqueous layer was extracted with EtOAc (2×). The combined organic layers were washed once with brine, dried over sodium sulfate, filtered and concentrated under reduced pressure to afford a light yellow oil (331 mg) containing the title compound. The crude material was used in the next step without further purification. MS (ESI): 464.2 [M−H]⁻

### Step b) tert-butyl N-[(3R)-7-[5-(1-cyano-1-methyl-ethyl)-1,3,4-oxadiazol-2-yl]-8-fluoro-4-oxo-3,5-dihydro-2H-1,5-benzothiazepin-3-yl]carbamate

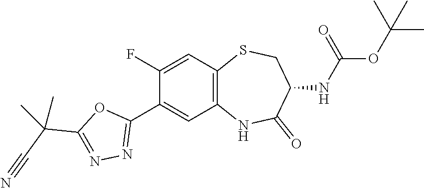 [SMILES: CC(C)(C)OC(=O)N[C@H]1CSc2cc(F)c(-c3nnc(C(C)(C)C#N)o3)cc2NC1=O]

The title compound was prepared from tert-butyl N-[(3R)-7-[[(2-cyano-2-methyl-propanoyl)amino]carbamoyl]-8-fluoro-4-oxo-3,5-dihydro-2H-1,5-benzothiazepin-3-yl]carbamate (188 mg, 403.87 mol) in analogy to general procedure 5b and was obtained as white solid (83.4 mg, 46%). MS (ESI): 392.1 [M+H-isobutene]⁺

### Step c) tert-butyl N-[(3R)-7-[5-(1-cyano-1-methyl-ethyl)-], 3,4-oxadiazol-2-yl]-8-fluoro-1,1,4-trioxo-3,5-dihydro-2H-1λ⁶,5-benzothiazepin-3-yl]carbamate

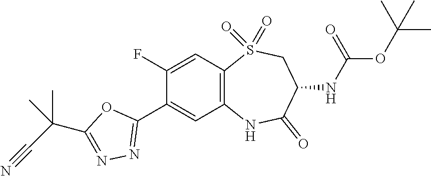 [SMILES: CC(C)(C)OC(=O)N[C@H]1CS(=O)(=O)c2cc(F)c(-c3nnc(C(C)(C)C#N)o3)cc2NC1=O]

The title compound was prepared from tert-butyl J-[(3R)-7-[5-(1-cyano-1-methyl-ethyl)-1,3,4-oxadiazol-2-yl]-8-fluoro-4-oxo-3,5-dihydro-2H-1,5-benzothiazepin-3-yl]carbamate (83.4 mg, 186.38 mol) in analogy to general procedure 10 and was obtained as white solid (47.2 mg, 49% yield). MS (ESI): 424.1 [M+H-isobutene]⁺.

### Step d) tert-butyl N-[(3R)-7-[5-(1-cyano-1-methyl-ethyl)-1,3,4-oxadiazol-2-yl]-8-fluoro-1,1,4-trioxo-5-[[4-[5-(trifluoromethyl)-2-pyridyl]phenyl]methyl]-2,3-dihydro-1λ⁶, 5-benzothiazepin-3-yl]carbamate

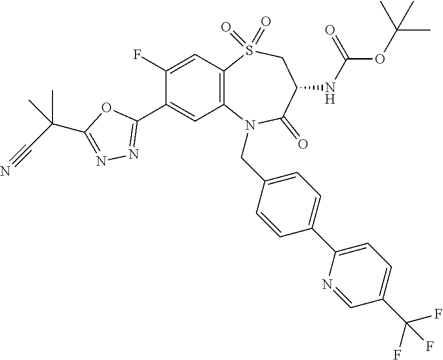 [SMILES: CC(C)(C)OC(=O)N[C@H]1CS(=O)(=O)c2cc(F)c(-c3nnc(C(C)(C)C#N)o3)cc2N(Cc2ccc(-c3ccc(C(F)(F)F)cn3)cc2)C1=O]

The title compound was prepared from tert-butyl N-[(3R)-7-[5-(1-cyano-1-methyl-ethyl)-1,3,4-oxadiazol-2-yl]-8-fluoro-1,1,4-trioxo-3,5-dihydro-2H-1λ⁶,5-benzothiazepin-3-yl]carbamate (47.2 mg, 0.091 mmol) and 2-[4-(bromomethyl)phenyl]-5-(trifluoromethyl)pyridine (42.94 mg, 135.9 mol, 1.5 eq, CAS 1056641-21-4) in analogy to general procedure 1a and was obtained as white solid (29.1 mg, 45%). MS (ESI): 715.5 [M+H]⁺

### Step e) 2-[5-[(3R)-3-amino-8-fluoro-1,1,4-trioxo-5-[[4-[5-(trifluoromethyl)-2-pyridyl]phenyl]methyl]-2,3-dihydro-1λ⁶,5-benzothiazepin-7-yl]-1,3,4-oxadiazol-2-yl]-2-methyl-propanenitrile

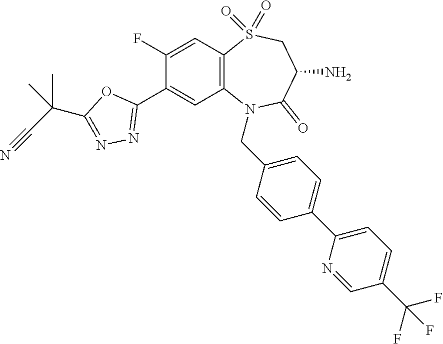 [SMILES: CC(C)(C#N)c1nnc(-c2cc3c(cc2F)S(=O)(=O)C[C@H](N)C(=O)N3Cc2ccc(-c3ccc(C(F)(F)F)cn3)cc2)o1]

The title compound was prepared from tert-butyl N-[(3R)-7-[5-(1-cyano-1-methyl-ethyl)-1,3,4-oxadiazol-2-yl]-8-fluoro-1,1,4-trioxo-5-[[4-[5-(trifluoromethyl)-2-pyridyl]phenyl]methyl]-2,3-dihydro-1λ⁶,5-benzothiazepin-3-yl]carbamate (29 mg, 40.58 mol) in analogy to general procedure 11a and was obtained as off-white solid (21 mg, 80%), as hydrochloride salt. MS (ESI): 659.2 [M−H+HCO₂H]⁻

### Example 3

### (3R)-3-amino-7-[5-[(3,3-difluoro-1-methyl-cyclobutyl)amino]-1,3,4-oxadiazol-2-yl]-8-fluoro-1,1-dioxo-5-[[4-[5-(trifluoromethyl)-1,2,4-oxadiazol-3-yl]phenyl]methyl]-2,3-dihydro-1α⁶,5-benzothiazepin-4-one

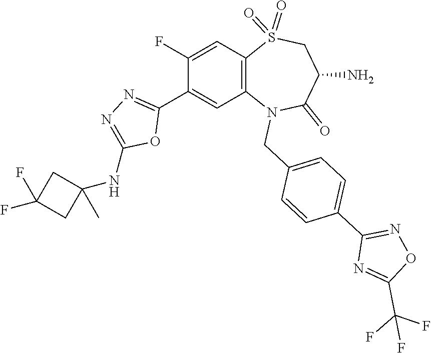 [SMILES: CC1(Nc2nnc(-c3cc4c(cc3F)S(=O)(=O)C[C@H](N)C(=O)N4Cc3ccc(-c4noc(C(F)(F)F)n4)cc3)o2)CC(F)(F)C1]

### Step a) methyl (3R)-3-(tert-butoxycarbonylamino)-8-fluoro-4-oxo-5-[[4-[5-(trifluoromethyl)-1,2,4-oxadiazol-3-yl]phenyl]methyl]-2,3-dihydro-1,5-benzothiazepine-7-carboxylate

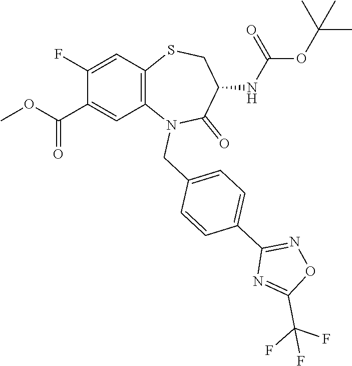 [SMILES: COC(=O)c1cc2c(cc1F)SC[C@H](NC(=O)OC(C)(C)C)C(=O)N2Cc1ccc(-c2noc(C(F)(F)F)n2)cc1]

The title compound was prepared from methyl (3R)-3-(tert-butoxycarbonylamino)-8-fluoro-4-oxo-3,5-dihydro-2H-1,5-benzothiazepine-7-carboxylate (500.0 mg, 1.35 mmol, 1.0 eq, CAS 202449-38-7) and 3-[4-(bromomethyl)phenyl]-5-(trifluoromethyl)-1,2,4-oxadiazole (414.51 mg, 1.35 mmol, 1.0 eq, CAS 2093101-98-3), in analogy to general procedure 1a and was obtained as light yellow foam (749 mg, 1.26 mmol, 93% yield. MS (ESI): 541.1 [M-isobutene-H]⁺

### Step b) methyl (3R)-3-(tert-butoxycarbonylamino)-8-fluoro-1,1,4-trioxo-5-[[4-[5-(trifluoromethyl)-1,2,4-oxadiazol-3-yl]phenyl]methyl]-2,3-dihydro-1λ⁶, 5-benzothiazepine-7-carboxylate

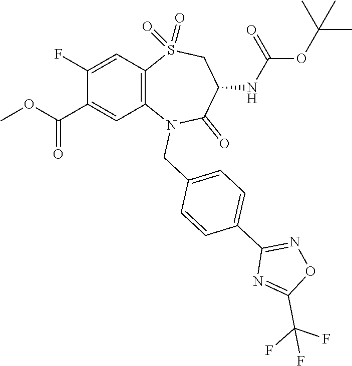 [SMILES: COC(=O)c1cc2c(cc1F)S(=O)(=O)C[C@H](NC(=O)OC(C)(C)C)C(=O)N2Cc1ccc(-c2noc(C(F)(F)F)n2)cc1]

The title compound was prepared from methyl (3R)-3-(tert-butoxycarbonylamino)-8-fluoro-4-oxo-5-[[4-[5-(trifluoromethyl)-1,2,4-oxadiazol-3-yl]phenyl]methyl]-2,3-dihydro-1,5-benzothiazepine-7-carboxylate (749 mg, 1.26 mmol) in analogy to general procedure 10 and was obtained as white solid (780 mg, 1.24 mmol, 99% yield). MS (ESI): 572.9 [M-isobutene+H]⁺

### Step c) (3R)-3-(tert-butoxycarbonylamino)-8-fluoro-1,1,4-trioxo-5-[[4-[5-(trifluoromethyl)-1,2,4-oxadiazol-3-yl]phenyl]methyl]-2,3-dihydro-1λ⁶, 5-benzothiazepine-7-carboxylic acid

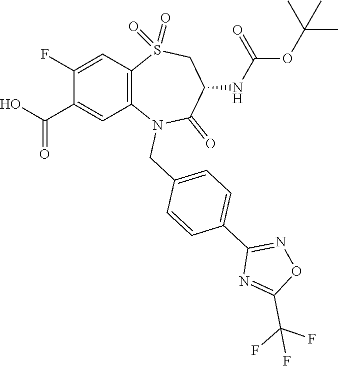 [SMILES: CC(C)(C)OC(=O)N[C@H]1CS(=O)(=O)c2cc(F)c(C(=O)O)cc2N(Cc2ccc(-c3noc(C(F)(F)F)n3)cc2)C1=O]

The title compound was prepared from methyl (3R)-3-(tert-butoxycarbonylamino)-8-fluoro-1,1,4-trioxo-5-[[4-[5-(trifluoromethyl)-1,2,4-oxadiazol-3-yl]phenyl]methyl]-2,3-dihydro-1λ⁶,5-benzothiazepine-7-carboxylate (200 mg, 0.32 mmol) in analogy to general procedure 2 and was obtained as white powder (170 mg, 0.28 mmol, 61% yield). MS (ESI): 514.8 [M−Boc+H]⁺

### Step d) tert-butyl N-[(3R)-8-fluoro-7-(hydrazinecarbonyl)-1,1,4-trioxo-5-[[4-[5-(trifluoromethyl)-1,2,4-oxadiazol-3-yl]phenyl]methyl]-2,3-dihydro-1λ⁶, 5-benzothiazepin-3-yl]carbamate

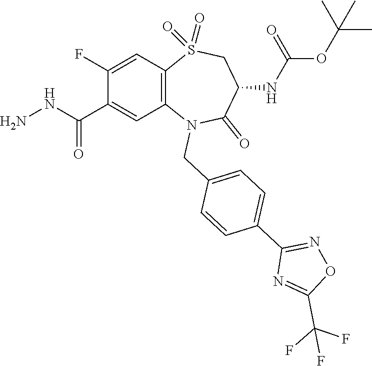 [SMILES: CC(C)(C)OC(=O)N[C@H]1CS(=O)(=O)c2cc(F)c(C(=O)NN)cc2N(Cc2ccc(-c3noc(C(F)(F)F)n3)cc2)C1=O]

The title compound was prepared from (3R)-3-(tert-butoxycarbonylamino)-8-fluoro-1,1,4-trioxo-5-[[4-[5-(trifluoromethyl)-1,2,4-oxadiazol-3-yl]phenyl]methyl]-2,3-dihydro-1λ⁶,5-benzothiazepine-7-carboxylic acid (3700 mg, 6.02 mmol) in analogy to general procedure 3 and was obtained as (2500 mg, 3.98 mmol, 66% yield). MS (ESI): 573.0 [M-isobutene+H]⁺

### Step e) tert-butyl N-[(3R)-8-fluoro-1,1,4-trioxo-7-(2-oxo-3H-1,3,4-oxadiazol-5-yl)-5-[[4-[5-(trifluoromethyl)-1,2,4-oxadiazol-3-yl]phenyl]methyl]-2,3-dihydro-1λ⁶, 5-benzothiazepin-3-yl]carbamate

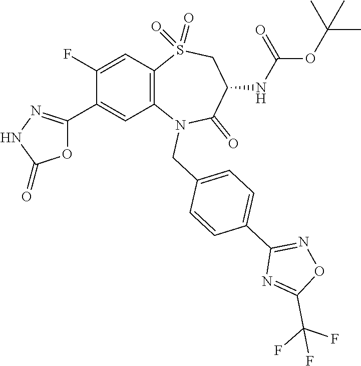 [SMILES: CC(C)(C)OC(=O)N[C@H]1CS(=O)(=O)c2cc(F)c(-c3n[nH]c(=O)o3)cc2N(Cc2ccc(-c3noc(C(F)(F)F)n3)cc2)C1=O]

To a solution of tert-butyl N-[(3R)-8-fluoro-7-(hydrazinecarbonyl)-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (1000 mg, 1.59 mmol, 1.0 eq) and triethylamine (0.44 mL, 3.18 mmol, 2.0 eq) in THF (20 mL) was added CDI (387 mg, 2.39 mmol, 1.5 eq) at RT and the mixture was stirred for 3 h. The solution was poured into water (20 mL). The aqueous phase was extracted with EtOAc (3×). The combined organic phase was washed with brine (2×), dried over anhydrous sodium sulfate, filtered and concentrated under vacuum. The remaining crude was purified by silica gel chromatography on silica gel (30-100% EtOAc in petroleum ether) to afford the title compound (80 mg, 0.14 mmol, 76% yield). MS (ESI): 598.9 [M-isobutene+H]⁺

### Step f) tert-butyl N-[(3R)-7-[5-[(3,3-difluoro-1-methyl-cyclobutyl)amino]-1,3,4-oxadiazol-2-yl]-8-fluoro-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate

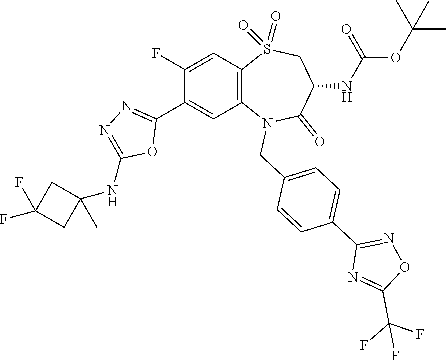 [SMILES: CC1(Nc2nnc(-c3cc4c(cc3F)S(=O)(=O)C[C@H](NC(=O)OC(C)(C)C)C(=O)N4Cc3ccc(-c4noc(C(F)(F)F)n4)cc3)o2)CC(F)(F)C1]

The title compound was prepared from tert-butyl N-[(3R)-8-fluoro-1,1,4-trioxo-7-(2-oxo-3H-1,3,4-oxadiazol-5-yl)-5-[[4-[5-(trifluoromethyl)-1,2,4-oxadiazol-3-yl]phenyl]methyl]-2,3-dihydro-1λ⁶,5-benzothiazepin-3-yl]carbamate (190.0 mg, 0.29 mmol, 1.0 eq) and 3,3-difluoro-1-methyl-cyclobutanamine hydrochloride (68.62 mg, 0.44 mmol, 1.5 eq) in analogy to general procedure 13 and was obtained as colorless foam (168.0 mg, 0.22 mmol, 76% yield). MS (ESI): 758.1 [M+H]⁺

### Step g) (3R)-3-amino-7-[5-[(3,3-difluoro-1-methyl-cyclobutyl)amino]-1,3,4-oxadiazol-2-yl]-8-fluoro-1,1-dioxo-5-[[4-[5-(trifluoromethyl)-1,2,4-oxadiazol-3-yl]phenyl]methyl]-2,3-dihydro-1λ⁶,5-benzothiazepin-4-one

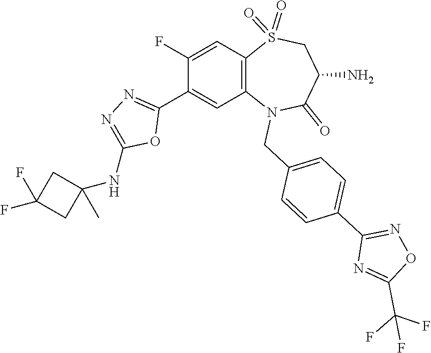 [SMILES: CC1(Nc2nnc(-c3cc4c(cc3F)S(=O)(=O)C[C@H](N)C(=O)N4Cc3ccc(-c4noc(C(F)(F)F)n4)cc3)o2)CC(F)(F)C1]

The title compound was prepared from tert-butyl N-[(3R)-7-[5-[(3,3-difluoro-1-methyl-cyclobutyl)amino]-1,3,4-oxadiazol-2-yl]-8-fluoro-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (168 mg, 0.22 mmol) in analogy to general procedure 11c and was obtained as white solid, as hydrochloride salt (131.6 mg, 0.19 mmol, 85% yield). MS (ESI): 657.8 [M+H]+

The examples of the following table were prepared in analogy to Example 3, using the appropriate amine building block.

| | | | | MS, ESI: |
|---|---|---|---|---|
| Ex | Structure | Systematic Name | Building Block | m/z |
| 4 | 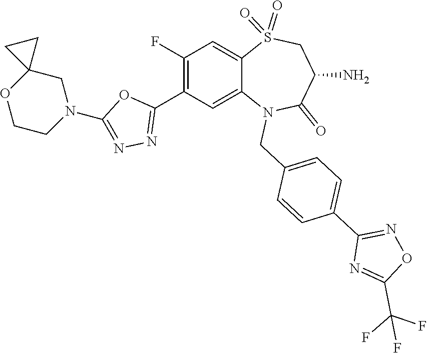 [SMILES: N[C@H]1CS(=O)(=O)c2cc(F)c(-c3nnc(N4CCOC5(CC5)C4)o3)cc2N(Cc2ccc(-c3noc(C(F)(F)F)n3)cc2)C1=O] | (3R)-3-amino-8- fluoro-7-[5-(4- oxa-7-azaspiro[2.5] octan-7-yl)-1,3,4- oxadiazol-2-yl]- 1,1-dioxo-5-[[4- [5-(trifluoromethyl)- 1,2,4-oxadiazol-3- yl]phenyl]methyl]- 2,3-dihydro-1λ⁶,5- benzothiazepin-4- one (*) | 4-oxa-7- azaspiro[2.5]octane hydrochloride (CAS 1427195-23-0) | 650.1 [M + H]⁺ |
| 5 | 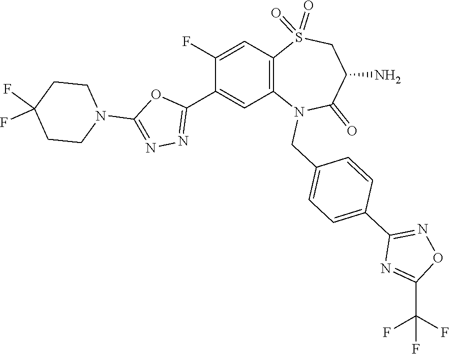 [SMILES: N[C@H]1CS(=O)(=O)c2cc(F)c(-c3nnc(N4CCC(F)(F)CC4)o3)cc2N(Cc2ccc(-c3noc(C(F)(F)F)n3)cc2)C1=O] | (3R)-3-amino-7- [5-(4,4-difluoro-1- piperidyl)-1,3,4- oxadiazol-2-yl]-8- fluoro-1,1-dioxo- 5-[[4-[5- (trifluoromethyl)- 1,2,4-oxadiazol-3- yl]phenyl]methyl]- 2,3-dihydro-1λ⁶,5- benzothiazepin-4- one (*) | difluoropiperidine hydrochloride (CAS 144230-52-4) | 657.9 [M + H]⁺ |

(*) as hydrochloride salt

### Example 6

### (3R)-3-amino-8-fluoro-7-[5-(isopropylamino)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

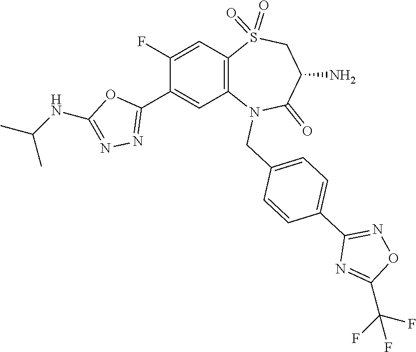 [SMILES: CC(C)Nc1nnc(-c2cc3c(cc2F)S(=O)(=O)C[C@H](N)C(=O)N3Cc2ccc(-c3noc(C(F)(F)F)n3)cc2)o1]

### Step a) tert-butyl N-[(3R)-8-fluoro-7-[5-(isopropylamino)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶, 5-benzothiazepin-3-yl]carbamate

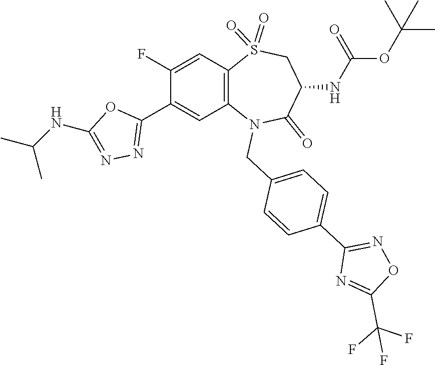 [SMILES: CC(C)Nc1nnc(-c2cc3c(cc2F)S(=O)(=O)C[C@H](NC(=O)OC(C)(C)C)C(=O)N3Cc2ccc(-c3noc(C(F)(F)F)n3)cc2)o1]

To a solution of tert-butyl N-[(3R)-8-fluoro-7-(hydrazinecarbonyl)-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (250.0 mg, 0.4 mmol, 1.0 eq, Example 3, step d) in DCM (5 mL) was added isopropyl isocyanate (0.07 mL, 0.68 mmol, 1.72 eq) at 0° C. The mixture was stirred for 3 h at RT. Then, 4-toluenesulfonyl chloride (130.4 mg, 0.68 mmol, 1.72 eq) and triethylamine (0.14 mL, 1.03 mmol, 2.6 eq) were added. The mixture was stirred for 1 h at RT. The reaction mixture was concentrated and purified by column chromatography on silica gel (25-75% EtOAc in petroleum ether) to afford the title compound as light yellow solid (142 mg, 0.2 mmol, 51% yield). MS (ESI): 696.0 [M+H]⁺

### Step b) (3R)-3-amino-8-fluoro-7-[5-(isopropylamino)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one

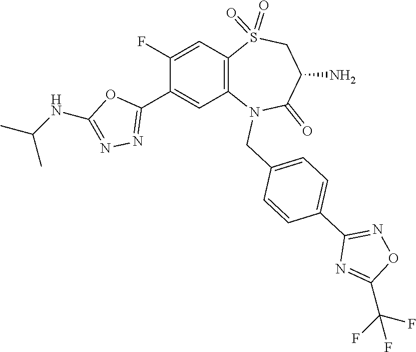 [SMILES: CC(C)Nc1nnc(-c2cc3c(cc2F)S(=O)(=O)C[C@H](N)C(=O)N3Cc2ccc(-c3noc(C(F)(F)F)n3)cc2)o1]

The title compound was prepared from tert-butyl N-[(3R)-8-fluoro-7-[5-(isopropylamino)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (122 mg, 0.18 mmol) in analogy to general procedure 11c and was obtained as white solid ((73.7 mg, 0.12 mmol, 70% yield). MS (ESI): 595.9 [M+H]⁺

### Example 7

### (3R)-3-amino-7-(5-tert-butyl-1,2,4-oxadiazol-3-yl)-8-fluoro-5-[[6-[4-(hydroxymethyl)phenyl]-3-pyridyl]methyl]-1,1-dioxo-2,3-dihydro-1λ⁶,5-benzothiazepin-4-one

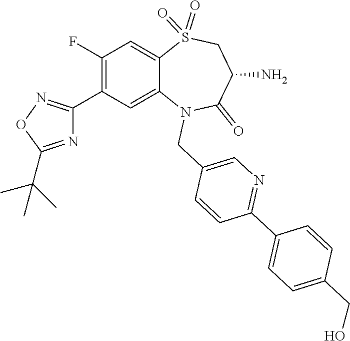 [SMILES: CC(C)(C)c1nc(-c2cc3c(cc2F)S(=O)(=O)C[C@H](N)C(=O)N3Cc2ccc(-c3ccc(CO)cc3)nc2)no1]

### Step a) (2R)-2-(tert-butoxycarbonylamino)-3-(4-cyano-5-fluoro-2-nitro-phenyl)sulfanyl-propanoic Acid

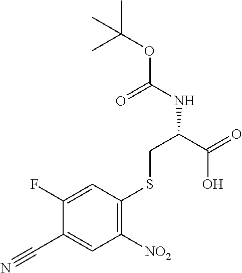 [SMILES: CC(C)(C)OC(=O)N[C@@H](CSc1cc(F)c(C#N)cc1[N+](=O)[O-])C(=O)O]

To a solution of 2,4-difluoro-5-nitro-benzonitrile (9.4 g, 50 mmol) and (tert-butoxycarbonyl)-L-cysteine (11.07 g, 50 mmol) in DCM (157 mL) was added DIPEA (17.48 mL, 100 mmol, Eq: 2). The reaction mixture was stirred for 24 hours at 22° C., diluted with DCM (40 mL) and washed once with 1N aqueous HCl solution and extracted twice with DCM. The combined organic layers were washed with brine, dried over sodium sulfate, filtered and concentrated in vacuo to yield the title compound as a yellow solid (23.5 g, 118% yield). MS (ESI): 286.1 [M-Boc+H]⁺.

### Step b) (2R)-3-(2-amino-4-cyano-5-fluoro-phenyl)sulfanyl-2-(tert-butoxycarbonylamino)propanoic Acid

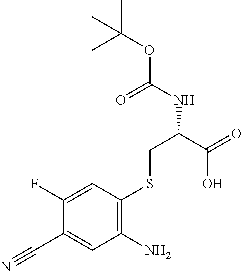 [SMILES: CC(C)(C)OC(=O)N[C@@H](CSc1cc(F)c(C#N)cc1N)C(=O)O]

To a solution of (2R)-2-(tert-butoxycarbonylamino)-3-(4-cyano-5-fluoro-2-nitro-phenyl)sulfanyl-propanoic acid (14.0 g, 36.3 mmol, 1.0 eq) in MeOH (140 mL) was added a solution of NH₄Cl (5.83 g, 109 mmol, 3.0 eq) in water (28 mL), followed by addition of Fe (10.71 mL, 145.31 mmol, 4.0 eq) portion wise. Then the mixture was stirred at 70° C. for 2 h. The reaction was allowed to cool to RT and then filtered through a celite plug, washing with MeOH (200 mL). The filtrate was concentrated to give (2R)-3-(2-amino-4-cyano-5-fluoro-phenyl)sulfanyl-2-(tert-butoxycarbonylamino)propanoic acid as a black solid (23 g, 48.1 mmol, 80% yield). MS (ESI): 300.1 [M-isobutene+H]⁺

### Step c) tert-butyl N-[(3R)-7-cyano-8-fluoro-4-oxo-3,5-dihydro-2H-1,5-benzothiazepin-3-yl]carbamate

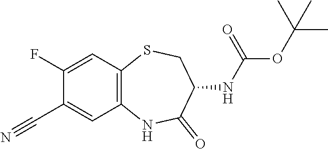 [SMILES: CC(C)(C)OC(=O)N[C@H]1CSc2cc(F)c(C#N)cc2NC1=O]

To a solution of (2R)-3-(2-amino-4-cyano-5-fluoro-phenyl)sulfanyl-2-(tert-butoxycarbonylamino)propanoic acid (15.0 g, 42.21 mmol, 1.0 eq) and N,N-diisopropylethylamine (14.7 mL, 84.42 mmol, 2.0 eq) in THF (300 mL) was added T₃P in EtOAc (40.29 g, 63.31 mmol, 1.5 eq) at RT and the mixture was stirred for 4 h. The reaction mixture was diluted with EtOAc (300 ml) and poured into water (600 ml). The layers were separated and the aqueous phase washed twice with EtOAc. The combined organic layers were washed with brine, dried over anhydrous sodium sulfate, filtered and concentrated. The remaining residue was purified by reverse phase preparative HPLC to afford tert-butyl N-[(3R)-7-cyano-8-fluoro-4-oxo-3,5-dihydro-2H-1,5-benzothiazepin-3-yl]carbamate as a light yellow solid (8.4 g, 24.9 mmol, 48% yield). MS (ESI): 282.1 [M-isobutene+H]⁺

### Step d) tert-butyl N-[(3R)-8-fluoro-7-[(Z)-N′-hydroxycarbamimidoyl]-4-oxo-3,5-dihydro-2H-1,5-benzothiazepin-3-yl]carbamate

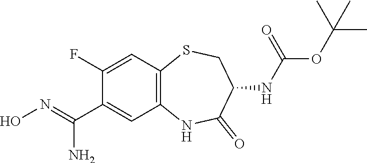 [SMILES: CC(C)(C)OC(=O)N[C@H]1CSc2cc(F)c(/C(N)=N/O)cc2NC1=O]

To a solution of tert-butyl N-[(3R)-7-cyano-8-fluoro-4-oxo-3,5-dihydro-2H-1,5-benzothiazepin-3-yl]carbamate(2R)-3-(2-amino-4-cyano-5-fluoro-phenyl)sulfanyl-2-(tert-butoxycarbonylamino)propanoic acid (200 mg, 0.59 mmol) in MeOH (2 mL) were added hydroxylamine hydrochloride (63.7 mg, 0.89 mmol, Eq: 1.5) and sodium bicarbonate (249 mg, 2.96 mmol, Eq: 5). The mixture was stirred for 16 hours at 70° C., cooled to RT, filtered and the filter cake was washed with DCM. The combined filtrates were concentrated in vacuo. The reaction was diluted with DCM and washed with water and brine. The organic layer was then dried over sodium sulfate, filtered and concentrated in vacuo to afford the title compound (444 mg, 1.19 mmol, 74% yield) as yellow solid. MS (ESI): 315.1 [M-isobutene+H]⁺

### Step e) [(Z)-[amino-[(3R)-3-(tert-butoxycarbonylamino)-8-fluoro-4-oxo-3,5-dihydro-2H-1,5-benzothiazepin-7-yl]methylene]amino]2,2-dimethylpropanoate

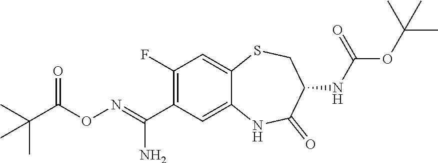 [SMILES: CC(C)(C)OC(=O)N[C@H]1CSc2cc(F)c(/C(N)=N/OC(=O)C(C)(C)C)cc2NC1=O]

The title compound was prepared from tert-butyl N-[(3R)-8-fluoro-7-[(Z)-N′-hydroxycarbamimidoyl]-4-oxo-3,5-dihydro-2H-1,5-benzothiazepin-3-yl]carbamate (1000 mg, 2.7 mmol) in analogy to general procedure 8b and was obtained as white solid (900 mg, 1.98 mmol, 73% yield. MS (ESI): 455.1 [M+H]⁺

### Step f) tert-butyl N-[(3R)-7-(5-tert-butyl-1,2,4-oxadiazol-3-yl)-8-fluoro-4-oxo-3,5-dihydro-2H-1,5-benzothiazepin-3-yl]carbamate

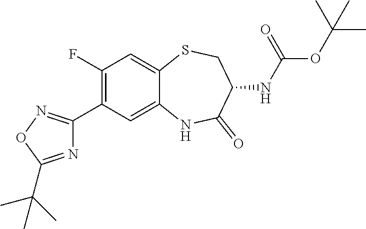 [SMILES: CC(C)(C)OC(=O)N[C@H]1CSc2cc(F)c(-c3noc(C(C)(C)C)n3)cc2NC1=O]

The title compound was prepared from [(Z)-[amino-[(3R)-3-(tert-butoxycarbonylamino)-8-fluoro-4-oxo-3,5-dihydro-2H-1,5-benzothiazepin-7-yl]methylene]amino]2,2-dimethylpropanoate (2.12 g, 4.75 mmol, 1 eq) in analogy to general procedure 9a and was obtained as orange solid (1.94 g, 89%). MS (ESI): 381.1 [M-isobutene+H]⁺

### Step g) tert-butyl N-[(3R)-7-(5-tert-butyl-1,2,4-oxadiazol-3-yl)-8-fluoro-1,1,4-trioxo-3,5-dihydro-2H-1λ⁶,5-benzothiazepin-3-yl]carbamate

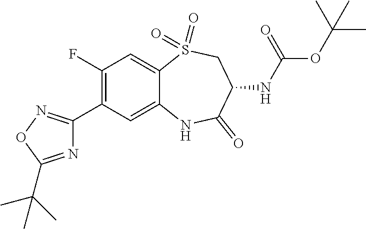 [SMILES: CC(C)(C)OC(=O)N[C@H]1CS(=O)(=O)c2cc(F)c(-c3noc(C(C)(C)C)n3)cc2NC1=O]

The title compound was prepared from tert-butyl N-[(3R)-7-(5-tert-butyl-1,2,4-oxadiazol-3-yl)-8-fluoro-4-oxo-3,5-dihydro-2H-1,5-benzothiazepin-3-yl]carbamate (500 mg, 1.09 mmol) in analogy to general procedure 10 and was obtained as light yellow solid (426 mg, 84%). MS (ESI): 413.2 [M+H]⁺

### Step h) tert-butyl N-[(3R)-5-[[6-[4-[[tert-butyl(dimethyl)silyl]oxymethyl]phenyl]-3-pyridyl]methyl]-7-(5-tert-butyl-1,2,4-oxadiazol-3-yl)-8-fluoro-1,1,4-trioxo-2,3-dihydro-1λ⁶, 5-benzothiazepin-3-yl]carbamate

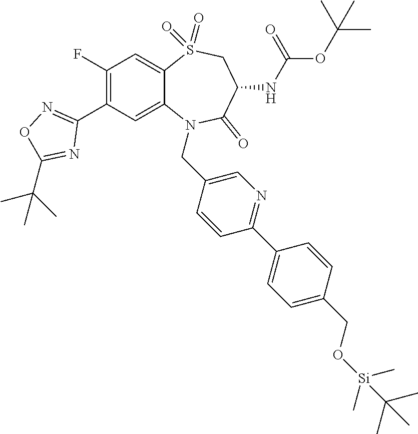 [SMILES: CC(C)(C)OC(=O)N[C@H]1CS(=O)(=O)c2cc(F)c(-c3noc(C(C)(C)C)n3)cc2N(Cc2ccc(-c3ccc(CO[Si](C)(C)C(C)(C)C)cc3)nc2)C1=O]

The title compound was prepared from tert-butyl N-[(3R)-7-(5-tert-butyl-1,2,4-oxadiazol-3-yl)-8-fluoro-1,1,4-trioxo-3,5-dihydro-2H-1λ⁶,5-benzothiazepin-3-yl]carbamate (35 mg, 0.075 mmol, 1.0 eq) and Intermediate 1 (92.27 mg, 0.075 mmol, 1.0 eq) in analogy to general procedure 1a and was obtained as light yellow solid (10 mg, 17%). MS (ESI): 780.5 [M+H]⁺

### Step i) (3R)-3-amino-7-(5-tert-butyl-1,2,4-oxadiazol-3-yl)-8-fluoro-5-[[6-[4-(hydroxymethyl)phenyl]-3-pyridyl]methyl]-1,1-dioxo-2,3-dihydro-1λ⁶, 5-benzothiazepin-4-one

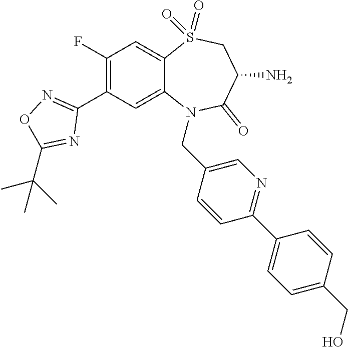 [SMILES: CC(C)(C)c1nc(-c2cc3c(cc2F)S(=O)(=O)C[C@H](N)C(=O)N3Cc2ccc(-c3ccc(CO)cc3)nc2)no1]

The title compound was prepared from tert-butyl N-[(3R)-5-[[6-[4-[[tert-butyl(dimethyl)silyl]oxymethyl]phenyl]-3-pyridyl]methyl]-7-(5-tert-butyl-1,2,4-oxadiazol-3-yl)-8-fluoro-1,1,4-trioxo-2,3-dihydro-1λ⁶,5-benzothiazepin-3-yl]carbamate (10 mg, 0.013 mmol) in analogy to general procedure 11a and was obtained as white solid (4.1 mg, 57%). MS (ESI): 566.3 [M+H]⁺

### Example 8

### (3R)-3-amino-7-[5-(3-aminooxetan-3-yl)-1,2,4-oxadiazol-3-yl]-8-fluoro-5-[[4-(4-methoxyphenyl)phenyl]methyl]-1,1-dioxo-2,3-dihydro-1λ⁶,5-benzothiazepin-4-one

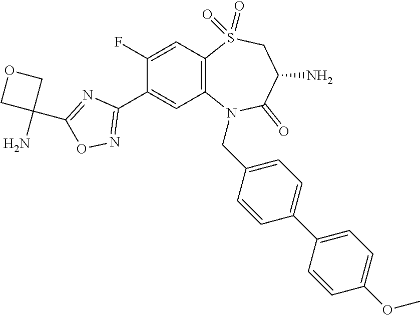 [SMILES: COc1ccc(-c2ccc(CN3C(=O)[C@@H](N)CS(=O)(=O)c4cc(F)c(-c5noc(C6(N)COC6)n5)cc43)cc2)cc1]

### Step a) tert-butyl N-[3-[3-[(3R)-3-(tert-butoxycarbonylamino)-8-fluoro-4-oxo-3,5-dihydro-2H-1,5-benzothiazepin-7-yl]-1,2,4-oxadiazol-5-yl]oxetan-3-yl]carbamate

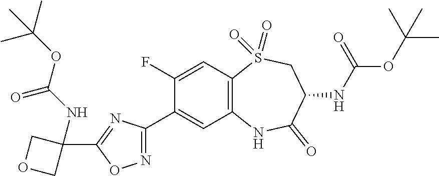 [SMILES: CC(C)(C)OC(=O)N[C@H]1CS(=O)(=O)c2cc(F)c(-c3noc(C4(NC(=O)OC(C)(C)C)COC4)n3)cc2NC1=O]

The title compound was prepared in analogy to general procedure 7b from tert-butyl N-[(3R)-8-fluoro-7-[(Z)-N′-hydroxycarbamimidoyl]-4-oxo-3,5-dihydro-2H-1,5-benzothiazepin-3-yl]carbamate (186 mg, 0.462 mmol, Example 7, step d) and 3-(tert-butoxycarbonylamino)oxetane-3-carboxylic acid (150 mg, 0.69 mmol, Eq: 1.5) and was obtained as alight yellow solid (166 mg, 0.3 mmol, 60% yield). MS (ESI): 550.5 [M−H]

### Step b) tert-butyl N-[3-[3-[(3R)-3-(tert-butoxycarbonylamino)-8-fluoro-5-[[4-(4-methoxyphenyl)phenyl]methyl]-4-oxo-2,3-dihydro-1,5-benzothiazepin-7-yl]-1,2,4-oxadiazol-5-yl]oxetan-3-yl]carbamate

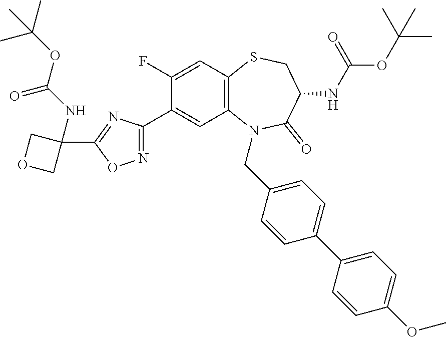 [SMILES: COc1ccc(-c2ccc(CN3C(=O)[C@@H](NC(=O)OC(C)(C)C)CSc4cc(F)c(-c5noc(C6(NC(=O)OC(C)(C)C)COC6)n5)cc43)cc2)cc1]

The title compound was prepared in analogy to general procedure 1a from tert-butyl N-[3-[3-[(3R)-3-(tert-butoxycarbonylamino)-8-fluoro-4-oxo-3,5-dihydro-2H-1,5-benzothiazepin-7-yl]-1,2,4-oxadiazol-5-yl]oxetan-3-yl]carbamate (40 mg, 0.067 mmol, Eq: 1) and 1-(chloromethyl)-4-(4-methoxyphenyl)benzene (CAS 93258-73-2) (23.5 mg, 0.1 mmol, Eq: 1.5) and was obtained as a white solid (45 mg, 82% yield). MS (ESI): 746.4 [M−H]

### Step c) tert-butyl N-[3-[3-[(3R)-3-(tert-butoxycarbonylamino)-8-fluoro-5-[[4-(4-methoxyphenyl)phenyl]methyl]-1,1,4-trioxo-2,3-dihydro-1λ⁶,5-benzothiazepin-7yl]-1,2,4-oxadiazol-5-yl]oxetan-3-yl]carbamate

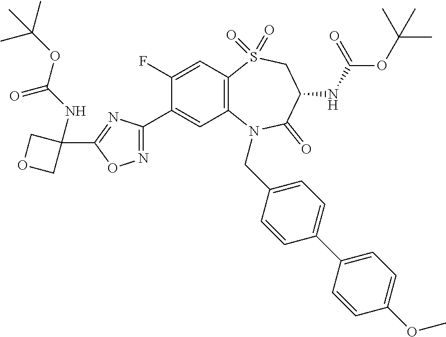

The title compound was prepared in analogy to general procedure 10 from tert-butyl N-[3-[3-[(3R)-3-(tert-butoxycarbonylamino)-8-fluoro-5-[[4-(4-methoxyphenyl)phenyl]methyl]-4-oxo-2,3-dihydro-1,5-benzothiazepin-7-yl]-1,2,4-oxadiazol-5-yl]oxetan-3-yl]carbamate (45 mg, 0.055 mmol) and was obtained as a white solid (30 mg, 69% yield). MS (ESI): 778.5 [M−H]

### Step d) (3R)-3-amino-7-[5-(3-aminooxetan-3-yl)-1,2,4-oxadiazol-3-yl]-8-fluoro-5-[[4-(4-methoxyphenyl)phenyl]methyl]-1,1-dioxo-2,3-dihydro-1λ⁶,5-benzothiazepin-4-one

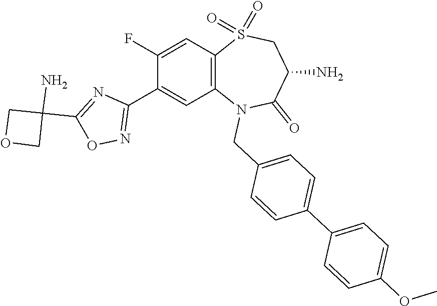 [SMILES: COc1ccc(-c2ccc(CN3C(=O)[C@@H](N)CS(=O)(=O)c4cc(F)c(-c5noc(C6(N)COC6)n5)cc43)cc2)cc1]

The title compound was prepared in analogy to general procedure 11a from tert-butyl N-[3-[3-[(3R)-3-(tert-butoxycarbonylamino)-8-fluoro-5-[[4-(4-methoxyphenyl)phenyl]methyl]-1,1,4-trioxo-2,3-dihydro-1λ⁶,5-benzothiazepin-7-yl]-1,2,4-oxadiazol-5-yl]oxetan-3-yl]carbamate (30 mg, 0.038 mmol) and was obtained as a white solid (9 mg, 40.3% yield). MS (ESI): 580.3 [M+H]⁺.

### Example 9 of the Following Table was Prepared in Analogy to Example 8 Using the Appropriate Benzyl Bromide Building Block

| | | | | MS, ESI: |
|---|---|---|---|---|
| Ex. | Structure | Systematic Name | Building Blocks | m/z |
| 9 | 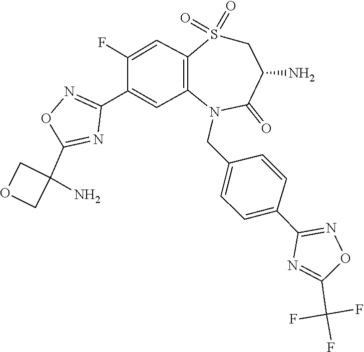 [SMILES: N[C@H]1CS(=O)(=O)c2cc(F)c(-c3noc(C4(N)COC4)n3)cc2N(Cc2ccc(-c3noc(C(F)(F)F)n3)cc2)C1=O] | (3R)-3-amino-7-[5- (3-aminooxetan-3- yl)-1,2,4- oxadiazol-3-yl]-8- fluoro-1,1-dioxo-5- [[4-[5- (trifluoromethyl)- 1,2,4-oxadiazol-3- yl]phenyl]methyl]- 2,3-dihydro-1λ⁶,5- benzothiazepin-4- one | 3-[4- (chloromethyl) phenyl]-5- (trifluoromethyl)- 1,2,4-oxadiazole (CAS 2246965- 50-2) | 610.3 [M + H]⁺ |

### Example 10

### (3R)-3-amino-7-[5-(3-aminooxetan-3-yl)-1,2,4-oxadiazol-3-yl]-8-fluoro-1,1-dioxo-5-[[4-[5-(trifluoromethyl)-2-pyridyl]phenyl]methyl]-2,3-dihydro-1λ⁶,5-benzothiazepin-4-one

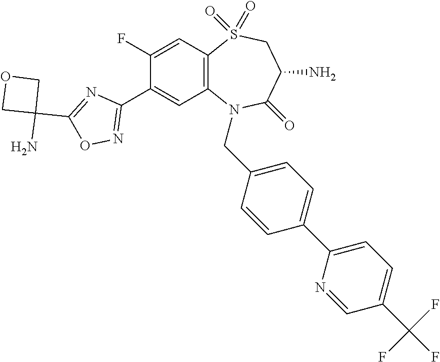 [SMILES: N[C@H]1CS(=O)(=O)c2cc(F)c(-c3noc(C4(N)COC4)n3)cc2N(Cc2ccc(-c3ccc(C(F)(F)F)cn3)cc2)C1=O]

### Step a) tert-butyl N-[3-[3-[(3R)-3-(tert-butoxycarbonylamino)-8-fluoro-1,1,4-trioxo-3,5-dihydro-2H-1λ⁶,5-benzothiazepin-7-yl]-1,2,4-oxadiazol-5-yl]oxetan-3-yl]carbamate

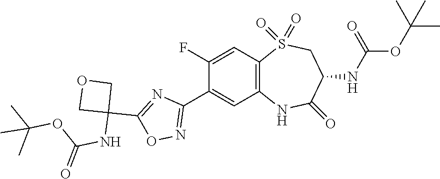 [SMILES: CC(C)(C)OC(=O)N[C@H]1CS(=O)(=O)c2cc(F)c(-c3noc(C4(NC(=O)OC(C)(C)C)COC4)n3)cc2NC1=O]

The title compound was prepared in analogy to general procedure 10 from tert-butyl N-[3-[3-[(3R)-3-(tert-butoxycarbonylamino)-8-fluoro-4-oxo-3,5-dihydro-2H-1,5-benzothiazepin-7-yl]-1,2,4-oxadiazol-5-yl]oxetan-3-yl]carbamate (Example 8, step a) (40 mg, 0.067 mmol) and was obtained as white solid (29 mg, 73% yield). MS (ESI): 472.2 [M-isobutene+H]⁺.

### Step b) tert-butyl N-[3-[3-[(3R)-3-(tert-butoxycarbonylamino)-8-fluoro-1,1,4-trioxo-5-[[4-[5-(trifluoromethyl)-2-pyridyl]phenyl]methyl]-2,3-dihydro-1λ⁶,5-benzothiazepin-7yl]-1,2,4-oxadiazol-5-yl]oxetan-3-yl]carbamate

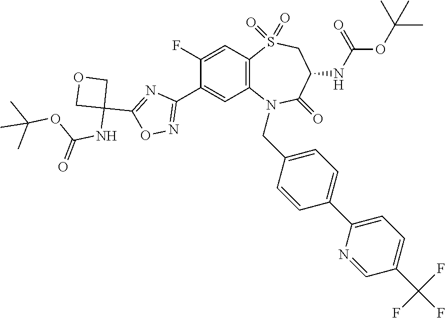 [SMILES: CC(C)(C)OC(=O)N[C@H]1CS(=O)(=O)c2cc(F)c(-c3noc(C4(NC(=O)OC(C)(C)C)COC4)n3)cc2N(Cc2ccc(-c3ccc(C(F)(F)F)cn3)cc2)C1=O]

The title compound was prepared in analogy to general procedure 1a from tert-butyl N-[3-[3-[(3R)-3-(tert-butoxycarbonylamino)-8-fluoro-1,1,4-trioxo-3,5-dihydro-2H-1λ⁶,5-benzothiazepin-7-yl]-1,2,4-oxadiazol-5-yl]oxetan-3-yl]carbamate (29 mg, 0.050 mmol) and 2-[4-(chloromethyl)phenyl]-5-(trifluoromethyl)pyridine (20.25 mg, 0.075 mmol, Eq: 1.5) and was obtained as a white solid (10 mg, 18% yield). MS (ESI): 819.6 [M+H]⁺.

### Step c) (3R)-3-amino-7-[5-(3-aminooxetan-3-yl)-1,2,4-oxadiazol-3-yl]-8-fluoro-1,1-dioxo-5-[[4-[5-(trifluoromethyl)-2-pyridyl]phenyl]methyl]-2,3-dihydro-1λ⁶,5-benzothiazepin-4-one

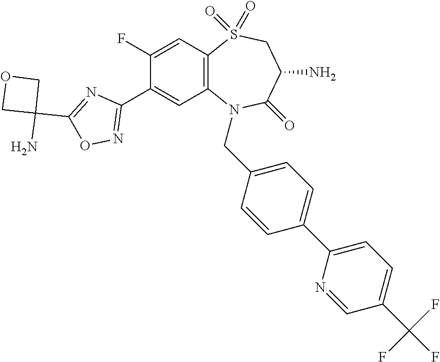 [SMILES: N[C@H]1CS(=O)(=O)c2cc(F)c(-c3noc(C4(N)COC4)n3)cc2N(Cc2ccc(-c3ccc(C(F)(F)F)cn3)cc2)C1=O]

The title compound was prepared in analogy to general procedure 11a from tert-butyl N-[3-[3-[(3R)-3-(tert-butoxycarbonylamino)-8-fluoro-1,1,4-trioxo-5-[[4-[5-(trifluoromethyl)-2-pyridyl]phenyl]methyl]-2,3-dihydro-1λ⁶,5-benzothiazepin-7-yl]-1,2,4-oxadiazol-5-yl]oxetan-3-yl]carbamate (10 mg, 0.01 mmol) and was obtained as a white solid (3 mg, 47% yield) MS (ESI): 619.3 [M+H]⁺.

### Example 11

### (3R)-3-amino-8-fluoro-7-[5-(4-oxa-7-azaspiro[2.5]octan-7-yl)-1,2,4-oxadiazol-3-yl]-1,1-dioxo-5-[[6-[4-(trifluoromethyl)phenyl]-3-pyridyl]methyl]-2,3-dihydro-1λ⁶,5-benzothiazepin-4-one

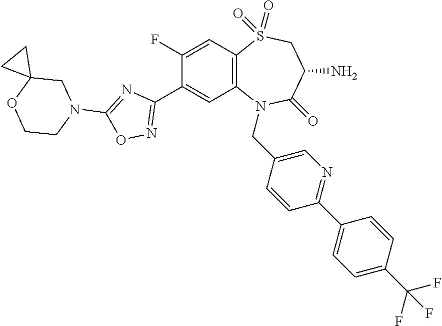 [SMILES: N[C@H]1CS(=O)(=O)c2cc(F)c(-c3noc(N4CCOC5(CC5)C4)n3)cc2N(Cc2ccc(-c3ccc(C(F)(F)F)cc3)nc2)C1=O]

### Step a) tert-butyl N-[(3R)-8-fluoro-4-oxo-7-(5-oxo-4H-1,2,4-oxadiazol-3-yl)-3,5-dihydro-2H-1,5-benzothiazepin-3-yl]carbamate

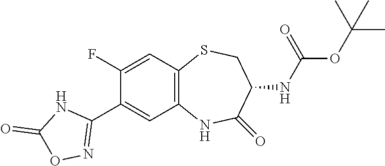 [SMILES: CC(C)(C)OC(=O)N[C@H]1CSc2cc(F)c(-c3noc(=O)[nH]3)cc2NC1=O]

To the solution of tert-butyl N-[(3R)-8-fluoro-7-[(Z)-N′-hydroxycarbamimidoyl]-4-oxo-3,5-dihydro-2H-1,5-benzothiazepin-3-yl]carbamate (580 mg, 1.57 mmol, 1.0 eq, Example 7, step d) and DIPEA (0.82 mL, 4.7 mmol, 3.0 eq) in DMF (10 mL) was added N.N′-carbonyldiimidazole (380.9 mg, 2.35 mmol, 1.5 eq) at 0° C. The mixture was stirred at 50° C. for 16 h. The mixture was poured into water (5 mL) and the pH adjusted to pH 4 with 2M HCl. The aqueous phase was extracted with EtOAc (10 mL×3). The combined organic phase was washed with brine (30 mL×3), dried over anhydrous sodium sulfate, filtered and concentrated under reduced pressure. The residue was triturated with (5 ml, 10% EtOAc in petroleum ether) for 10 min, then filtered and the filter cake dried in vacuo to afford the title compound (190 mg, 0.48 mmol, 22% yield) as light yellow solid. MS (ESI): 341.1 [M-isobutene+H]⁺

### Step b) tert-butyl N-[(3R)-8-fluoro-1,1,4-trioxo-7-(5-oxo-4H-1,2,4-oxadiazol-3-yl)-3,5-dihydro-2H-1λ⁶,5-benzothiazepin-3-yl]carbamate

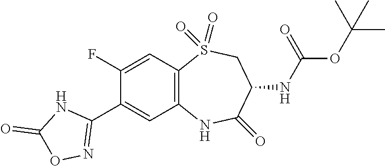 [SMILES: CC(C)(C)OC(=O)N[C@H]1CS(=O)(=O)c2cc(F)c(-c3noc(=O)[nH]3)cc2NC1=O]

The title compound was prepared from tert-butyl N-[(3R)-8-fluoro-7-(5-hydroxy-1,2,4-oxadiazol-3-yl)-4-oxo-3,5-dihydro-2H-1,5-benzothiazepin-3-yl]carbamate (20 mg, 0.05 mmol) in analogy to general procedure 10 and was obtained as light yellow solid (18 mg, 0.04 mmol, 80% yield). MS (ESI): 329.0 [M-Boc+H]⁺

### Step c) tert-butyl N-[(3R)-8-fluoro-7-[5-(4-oxa-7-azaspiro[2.5]octan-7-yl)-1,2,4-oxadiazol-3-yl]-1,1,4-trioxo-3,5-dihydro-2H-1λ⁶,5-benzothiazepin-3-yl]carbamate

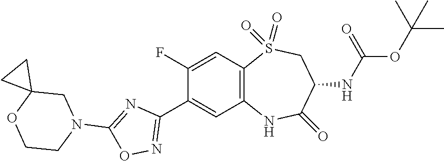 [SMILES: CC(C)(C)OC(=O)N[C@H]1CS(=O)(=O)c2cc(F)c(-c3noc(N4CCOC5(CC5)C4)n3)cc2NC1=O]

The title compound was prepared from tert-butyl N-[(3R)-8-fluoro-7-(5-hydroxy-1,2,4-oxadiazol-3-yl)-1,1,4-trioxo-3,5-dihydro-2H-1λ6,5-benzothiazepin-3-yl]carbamate (84 mg, 0.2 mmol, 1.0 eq) and 4-oxa-7-azaspiro[2.5]octane hydrochloride (44 mg, 0.29 mmol, 1.5 eq) in analogy to general procedure 13 and was obtained as white solid (79 mg, 0.15 mmol, 65% yield). MS (ESI): 468.1 [M-isobutene+H]⁺

### Step d) tert-butyl N-[(3R)-8-fluoro-7-[5-(4-oxa-7-azaspiro[2.5]octan-7yl)-1,2,4-oxadiazol-3-yl]-1,1,4-trioxo-5-[[6-[4-(trifluoromethyl)phenyl]-3-pyridyl]methyl]-2,3-dihydro-1λ⁶, 5-benzothiazepin-3-yl]carbamate

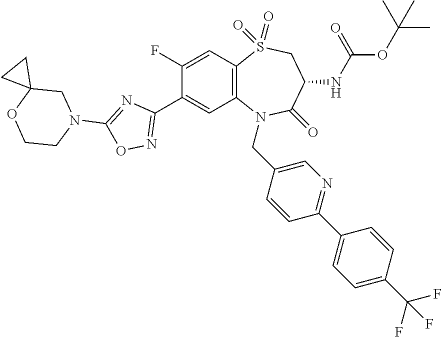 [SMILES: CC(C)(C)OC(=O)N[C@H]1CS(=O)(=O)c2cc(F)c(-c3noc(N4CCOC5(CC5)C4)n3)cc2N(Cc2ccc(-c3ccc(C(F)(F)F)cc3)nc2)C1=O]

The title compound was prepared from tert-butyl N-[(3R)-8-fluoro-7-[5-(4-oxa-7-azaspiro[2.5]octan-7-yl)-1,2,4-oxadiazol-3-yl]-1,1,4-trioxo-3,5-dihydro-2H-1λ6,5-benzothiazepin-3-yl]carbamate (94 mg, 0.18 mmol, 1.0 eq) and [6-[4-(trifluoromethyl)phenyl]-3-pyridyl]methanol (50 mg, 0.2 mmol, 1.1 eq, CAS 356058-13-4) in analogy to general procedure 1b and was obtained as white solid (60 mg, 0.08 mmol, 44% yield). MS (ESI): 688.2 [M+H]⁺

### Step e) (3R)-3-amino-8-fluoro-7-[5-(4-oxa-7-azaspiro[2.5]octan-7-yl)-1,2,4-oxadiazol-3-yl]-1,1-dioxo-5-[[6-[4-(trifluoromethyl)phenyl]-3-pyridyl]methyl]-2,3-dihydro-1λ⁶, 5-benzothiazepin-4-one

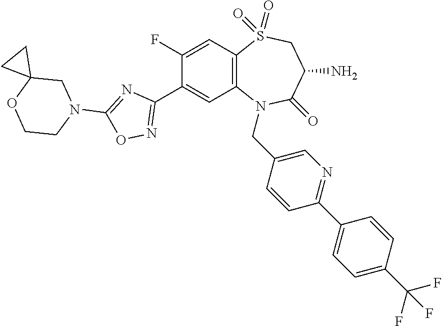 [SMILES: N[C@H]1CS(=O)(=O)c2cc(F)c(-c3noc(N4CCOC5(CC5)C4)n3)cc2N(Cc2ccc(-c3ccc(C(F)(F)F)cc3)nc2)C1=O]

The title compound was prepared from tert-butyl N-[(3R)-8-fluoro-7-[5-(4-oxa-7-azaspiro[2.5]octan-7-yl)-1,2,4-oxadiazol-3-yl]-1,1,4-trioxo-5-[[6-[4-(trifluoromethyl)phenyl]-3-pyridyl]methyl]-2,3-dihydro-1λ6,5-benzothiazepin-3-yl]carbamate (55 mg, 0.07 mmol) in analogy to general procedure 11c and was obtained as white solid. MS (ESI): 659.2 [M+H]⁺

The examples of the following table were prepared in analogy to Example 11 using the appropriate amine and benzyl bromide building block with the indicated general procedure.

| | | | | MS, ESI: |
|---|---|---|---|---|
| Ex. | Structure | Systematic Name | Building Blocks | m/z |
| 12 | 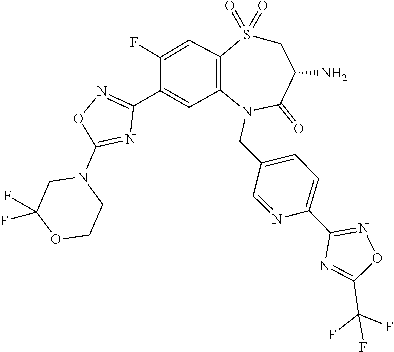 [SMILES: N[C@H]1CS(=O)(=O)c2cc(F)c(-c3noc(N4CCOC(F)(F)C4)n3)cc2N(Cc2ccc(-c3noc(C(F)(F)F)n3)nc2)C1=O] | (3R)-3-amino-7-[5-(2,2- difluoromorpholin-4- yl)-1,2,4-oxadiazol-3- yl]-8-fluoro-1,1-dioxo- 5-[[6-[5- (trifluoromethyl)-1,2,4- oxadiazol-3-yl]-3- pyridyl]methyl]-2,3- dihydro-1λ⁶,5- benzothiazepin-4-one | 2,2- difluoromorpholine (CAS 1263180- 85-3, step c), Intermediate 2 (step d, general procedure 1a) | 661.2 [M + H]⁺ |
| 13 | 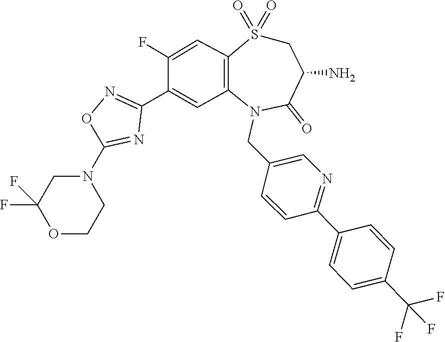 [SMILES: N[C@H]1CS(=O)(=O)c2cc(F)c(-c3noc(N4CCOC(F)(F)C4)n3)cc2N(Cc2ccc(-c3ccc(C(F)(F)F)cc3)nc2)C1=O] | (3R)-3-amino-7-[5-(2,2- difluoromorpholin-4- yl)-1,2,4-oxadiazol-3- yl]-8-fluoro-1,1-dioxo- 5-[[6-[4-(trifluoromethyl) phenyl]-3-pyridyl]methyl]- 2,3-dihydro-1λ⁶,5- benzothiazepin-4-one (*) | 2,2- difluoromorpholine (CAS 1263180- 85-3, step c) | 713.2 [M − H + HCO₂H] |

(*) as hydrochloride salt

### Example 14

### (3R)-3-amino-8-fluoro-7-[5-(4-oxa-7-azaspiro[2.5]octan-7-yl)-1,2,4-oxadiazol-3-yl]-1,1-dioxo-5-[[6-[5-(trifluoromethyl)-1,2,4-oxadiazol-3-yl]-3-pyridyl]methyl]-2,3-dihydro-1λ⁶,5-benzothiazepin-4-one

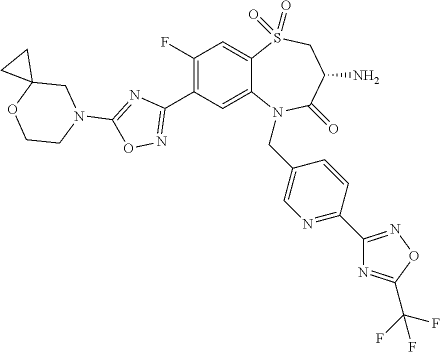 [SMILES: N[C@H]1CS(=O)(=O)c2cc(F)c(-c3noc(N4CCOC5(CC5)C4)n3)cc2N(Cc2ccc(-c3noc(C(F)(F)F)n3)nc2)C1=O]

### Step a) tert-butyl N-[(3R)-7-cyano-8-fluoro-1,1,4-trioxo-3,5-dihydro-2H-1λ⁶, 5-benzothiazepin-3-yl]carbamate

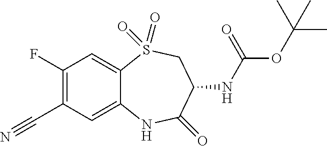 [SMILES: CC(C)(C)OC(=O)N[C@H]1CS(=O)(=O)c2cc(F)c(C#N)cc2NC1=O]

The title compound was prepared from tert-butyl N-[(3R)-7-cyano-8-fluoro-4-oxo-3,5-dihydro-2H-1,5-benzothiazepin-3-yl]carbamate (3.0 g, 8.89 mmol, Example 7, step c) in analogy to general procedure 10 and was obtained as white solid (3.2 g, 8.66 mmol, 88% yield). MS (ESI): 314.1 [M-isobutene+H]⁺

### Step b) tert-butyl N-[(3R)-7-cyano-8-fluoro-1,1,4-trioxo-5-[[6-[5-(trifluoromethyl)-1,2,4-oxadiazol-3-yl]-3-pyridyl]methyl]-2,3-dihydro-1λ⁶, 5-benzothiazepin-3-yl]carbamate

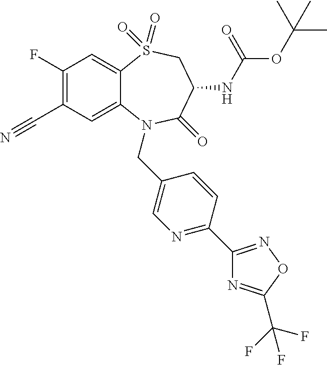 [SMILES: CC(C)(C)OC(=O)N[C@H]1CS(=O)(=O)c2cc(F)c(C#N)cc2N(Cc2ccc(-c3noc(C(F)(F)F)n3)nc2)C1=O]

The title compound was prepared from tert-butyl N-[(3R)-7-cyano-8-fluoro-1,1,4-trioxo-3,5-dihydro-2H-1λ6,5-benzothiazepin-3-yl]carbamate (100 mg, 0.27 mmol, 1.0 eq) and Intermediate 2 (166.7 mg, 0.3 mmol, 1.1 eq) in analogy to general procedure 1a and was obtained as white solid (100 mg, 0.17 mmol, 5900 yield). MS (ESI): 597.1 [M+H]⁺

### Step c) tert-butyl N-[(3R)-8-fluoro-7-[(Z)-N′-hydroxycarbamimidoyl]-1,1,4-trioxo-5-[[6-[3-(trifluoromethyl)-1,2,4-oxadiazol-5-yl]-3-pyridyl]methyl]-2,3-dihydro-1λ⁶, 5-benzothiazepin-3-yl]carbamate and tert-butyl N-[(3R)-8-fluoro-7-[(Z)-N′-hydroxycarbamimidoyl]-1,1,4-trioxo-5-[[6-[5-(trifluoromethyl)-1,2,4-oxadiazol-3-yl]-3-pyridyl]methyl]-2,3-dihydro-1λ⁶, 5-benzothiazepin-3-yl]carbamate

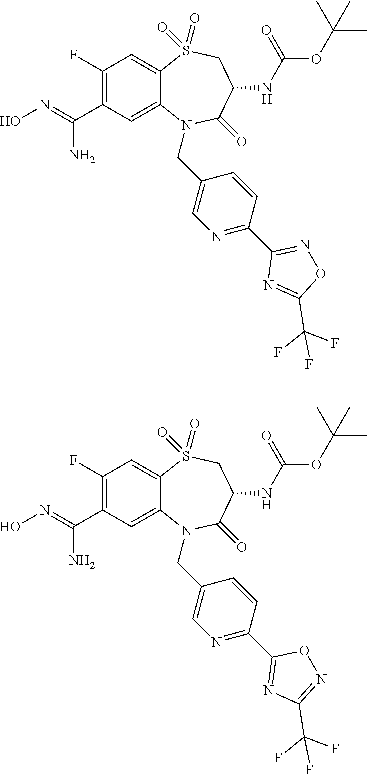

The title compounds were prepared from tert-butyl N-[(3R)-7-cyano-8-fluoro-1,1,4-trioxo-5-[[6-[5-(trifluoromethyl)-1,2,4-oxadiazol-3-yl]-3-pyridyl]methyl]-2,3-dihydro-1λ6,5-benzothiazepin-3-yl]carbamate (300 mg, 0.5 mmol, 1.0 eq) in analogy to general procedure 6 and were obtained as an inseparable mixture and light yellow solid (200 mg, 54% yield). MS (ESI): 630.2 [M+H]⁺

### Step d) tert-butyl N-[(3R)-8-fluoro-1,1,4-trioxo-7-(5-oxo-4H-1,2,4-oxadiazol-3-yl)-5-[[6-[3-(trifluoromethyl)-1,2,4-oxadiazol-5-yl]-3-pyridyl]methyl]-2,3-dihydro-1λ⁶, 5-benzothiazepin-3-yl]carbamate;tert-butyl and N-[(3R)-8-fluoro-1,1,4-trioxo-7-(5-oxo-4H-1,2,4-oxadiazol-3-yl)-5-[[6-[5-(trifluoromethyl)-1,2,4-oxadiazol-3-yl]-3-pyridyl]methyl]-2,3-dihydro-1λ⁶, 5-benzothiazepin-3-yl]carbamate

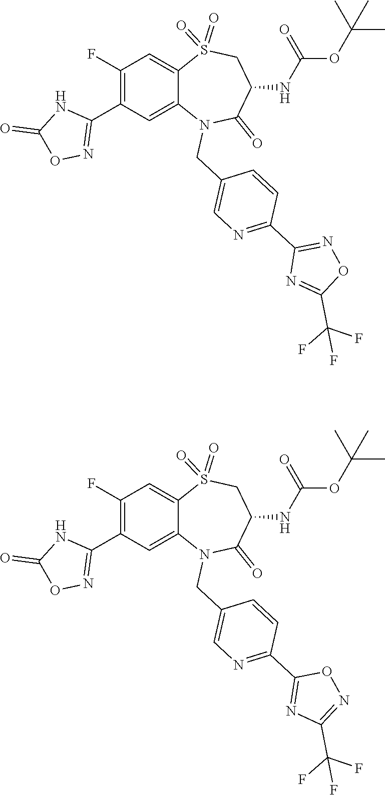 [SMILES: CC(C)(C)OC(=O)N[C@H]1CS(=O)(=O)c2cc(F)c(-c3noc(=O)[nH]3)cc2N(Cc2ccc(-c3noc(C(F)(F)F)n3)nc2)C1=O ; CC(C)(C)OC(=O)N[C@H]1CS(=O)(=O)c2cc(F)c(-c3noc(=O)[nH]3)cc2N(Cc2ccc(-c3nc(C(F)(F)F)no3)nc2)C1=O]

To a solution of a mixture of tert-butyl N-[(3R)-8-fluoro-7-[(Z)-N′-hydroxycarbamimidoyl]-1,1,4-trioxo-5-[[6-[3-(trifluoromethyl)-1,2,4-oxadiazol-5-yl]-3-pyridyl]methyl]-2,3-dihydro-1λ⁶,5-benzothiazepin-3-yl]carbamate and tert-butyl N-[(3R)-8-fluoro-7-[(Z)-N′-hydroxycarbamimidoyl]-1,1,4-trioxo-5-[[6-[5-(trifluoromethyl)-1,2,4-oxadiazol-3-yl]-3-pyridyl]methyl]-2,3-dihydro-1λ⁶,5-benzothiazepin-3-yl]carbamate (180 mg, 0.3 mmol, 1.0 eq) in DCM (9 ml) was added triethylamine (0.08 mL, 0.57 mmol, 2.0 eq) and N.N′-carbonyldiimidazole (69.5 mg, 0.43 mmol, 1.5 eq) RT and the mixture was stirred for 3 h. The reaction mixture was concentrated under reduced pressure. The remaining residue was dissolved in EtOAc (10 mL) and washed with brine (2×10 mL), dried over anhydrous sodium sulfate, and concentrated under reduced pressure. The remaining crude was purified by column chromatography on silica gel (30-100% EtOAc in petroleum ether) to afford an inseparable mixture of tert-butyl N-[(3R)-8-fluoro-1,1,4-trioxo-7-(5-oxo-4H-1,2,4-oxadiazol-3-yl)-5-[[6-[3-(trifluoromethyl)-1,2,4-oxadiazol-5-yl]-3-pyridyl]methyl]-2,3-dihydro-1λ⁶,5-benzothiazepin-3-yl]carbamate and tert-butyl N-[(3R)-8-fluoro-1,1,4-trioxo-7-(5-oxo-4H-1,2,4-oxadiazol-3-yl)-5-[[6-[5-(trifluoromethyl)-1,2,4-oxadiazol-3-yl]-3-pyridyl]methyl]-2,3-dihydro-1λ⁶,5-benzothiazepin-3-yl]carbamate (160 mg, 79% yield) as orange solid. MS (ESI): 656.2 [M+H]⁺

### Step e) tert-butyl N-[(3R)-8-fluoro-7-[5-(4-oxa-7-azaspiro[2.5]octan-7-yl)-1,2,4-oxadiazol-3-yl]-1,1,4-trioxo-5-[[6-[3-(trifluoromethyl)-1,2,4-oxadiazol-5-yl]-3-pyridyl]methyl]-2,3-dihydro-1λ⁶,5-benzothiazepin-3-yl]carbamate and tert-butyl N-[(3R)-8-fluoro-7-[5-(4-oxa-7-azaspiro[2.5]octan-7-yl)-1,2,4-oxadiazol-3-yl]-1,1,4-trioxo-5-[[6-[5-(trifluoromethyl)-1,2,4-oxadiazol-3-yl]-3-pyridyl]methyl]-2,3-dihydro-1λ⁶,5-benzothiazepin-3-yl]carbamate [mixture A] and tert-butyl N-[(3R)-8-fluoro-1,1,4-trioxo-7-(5-pyrrolidin-1-yl-1,2,4-oxadiazol-3-yl)-5-[[6-[3-(trifluoromethyl)-1,2,4-oxadiazol-5-yl]-3-pyridyl]methyl]-2,3-dihydro-1λ⁶, 5-benzothiazepin-3-yl]carbamate and tert-butyl N-[(3R)-8-fluoro-1,1,4-trioxo-7-(5-pyrrolidin-1-yl-1,2,4-oxadiazol-3-yl)-5-[[6-[5-(trifluoromethyl)-1,2,4-oxadiazol-3-yl]-3-pyridyl]methy]-2,3-dihydro-1λ⁶, 5-benzothiazepin-3-yl]carbamate [mixture B]

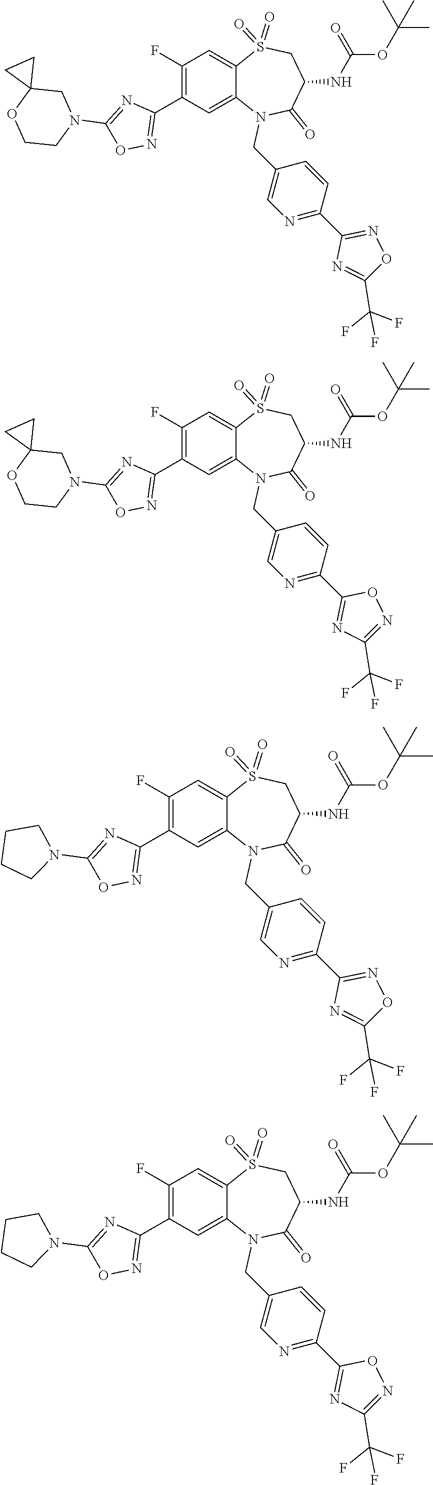

The title compounds were prepared from tert-butyl N-[(3R)-8-fluoro-1,1,4-trioxo-7-(5-oxo-4H-1,2,4-oxadiazol-3-yl)-5-[[6-[3-(trifluoromethyl)-1,2,4-oxadiazol-5-yl]-3-pyridyl]methyl]-2,3-dihydro-1λ⁶,5-benzothiazepin-3-yl]carbamate;tert-butyl and N-[(3R)-8-fluoro-1,1,4-trioxo-7-(5-oxo-4H-1,2,4-oxadiazol-3-yl)-5-[[6-[5-(trifluoromethyl)-1,2,4-oxadiazol-3-yl]-3-pyridyl]methyl]-2,3-dihydro-1λ⁶,5-benzothiazepin-3-yl]carbamate (60 mg, 0.09 mmol, 1.0 eq) and 4-oxa-7-azaspiro[2.5]octane; (15.06 mg, 0.1 mmol, 1.1 eq) in analogy to general procedure 13 and were obtained as an inseparable mixture and white solid [mixture A](30 mg, 23%, MS (ESI): 751.2 [M+H]⁺) and as an inseparable mixture and white solid [mixture B](20 mg, 19%, MS (ESI): 709.3 [M+H]⁺).

### Step f) (3R)-3-amino-8-fluoro-7-[5-(4-oxa-7-azaspiro[2.5]octan-7-yl)-1,2,4-oxadiazol-3-yl]-1,1-dioxo-5-[[6-[5-(trifluoromethyl)-1,2,4-oxadiazol-3-yl]-3-pyridyl]methyl]-2,3-dihydro-1λ⁶,5-benzothiazepin-4-one

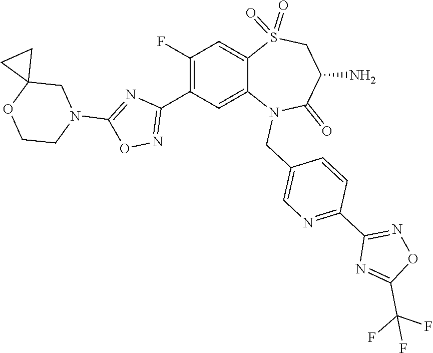 [SMILES: N[C@H]1CS(=O)(=O)c2cc(F)c(-c3noc(N4CCOC5(CC5)C4)n3)cc2N(Cc2ccc(-c3noc(C(F)(F)F)n3)nc2)C1=O]

To a solution of tert-butyl N-[(3R)-8-fluoro-7-[5-(4-oxa-7-azaspiro[2.5]octan-7-yl)-1,2,4-oxadiazol-3-yl]-1,1,4-trioxo-5-[[6-[3-(trifluoromethyl)-1,2,4-oxadiazol-5-yl]-3-pyridyl]methyl]-2,3-dihydro-1λ⁶,5-benzothiazepin-3-yl]carbamate and tert-butyl N-[(3R)-8-fluoro-7-[5-(4-oxa-7-azaspiro[2.5]octan-7-yl)-1,2,4-oxadiazol-3-yl]-1,1,4-trioxo-5-[[6-[5-(trifluoromethyl)-1,2,4-oxadiazol-3-yl]-3-pyridyl]methyl]-2,3-dihydro-1λ⁶,5-benzothiazepin-3-yl]carbamate [mixture A](30 mg, 0.04 mmol, 1.0 eq) in EtOAc (1 mL) was added 4N HCl in EtOAc (1.0 mL, 4.0 mmol, 100 eq) at 0° C. and the mixture was stirred at RT for 1 h. The mixture was concentrated under reduced pressure and the remaining residue was purified by chiral SFC (retention time 1.77 min; conditions: column Regis (S,S) Whelk-O 1, 250 mm*25 mm I.D., 10 μm, mobile phase: phase A for CO₂, phase B for EtOH (0.1% NH₃(aq)), eluent 40% EtOH (0.1% NH₃(aq)) in CO₂, flow rate 75 mL/min, detector: PDA) to afford pure title compound as white solid (7 mg) which was dissolved in EtOAc (1 ml). To this solution was added 4N HCl in EtOAc (1.0 mL, 4 mmol) and stirred for 1 h. The mixture was then concentrated under reduced pressure to afford the title compound (7.0 mg, 86% yield) as white solid, as hydrochloride salt. MS (ESI): 651.1 [M+H]⁺

### Example 15

### (3R)-3-amino-8-fluoro-1,1-dioxo-7-(5-pyrrolidin-1-yl-1,2,4-oxadiazol-3-yl)-5-[[6-[5-(trifluoromethyl)-1,2,4-oxadiazol-3-yl]-3-pyridyl]methyl]-2,3-dihydro-1λ⁶,5-benzothiazepin-4-one

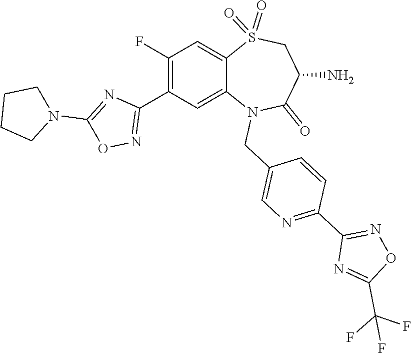 [SMILES: N[C@H]1CS(=O)(=O)c2cc(F)c(-c3noc(N4CCCC4)n3)cc2N(Cc2ccc(-c3noc(C(F)(F)F)n3)nc2)C1=O]

### Step a) (3R)-3-amino-8-fluoro-1,1-dioxo-7-(5-pyrrolidin-1-yl-1,2,4-oxadiazol-3-yl)-5-[[6-[5-(trifluoromethyl)-1,2,4-oxadiazol-3-yl]-3-pyridyl]methyl]-23-dihydro-1λ⁶, 5-benzothiazepin-4-one

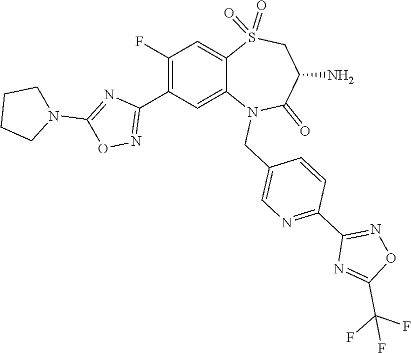 [SMILES: N[C@H]1CS(=O)(=O)c2cc(F)c(-c3noc(N4CCCC4)n3)cc2N(Cc2ccc(-c3noc(C(F)(F)F)n3)nc2)C1=O]

To a solution of tert-butyl N-[(3R)-8-fluoro-1,1,4-trioxo-7-(5-pyrrolidin-1-yl-1,2,4-oxadiazol-3-yl)-5-[[6-[3-(trifluoromethyl)-1,2,4-oxadiazol-5-yl]-3-pyridyl]methyl]-2,3-dihydro-1λ⁶,5-benzothiazepin-3-yl]carbamate and tert-butyl N-[(3R)-8-fluoro-1,1,4-trioxo-7-(5-pyrrolidin-1-yl-1,2,4-oxadiazol-3-yl)-5-[[6-[5-(trifluoromethyl)-1,2,4-oxadiazol-3-yl]-3-pyridyl]methyl]-2,3-dihydro-1λ⁶,5-benzothiazepin-3-yl]carbamate [mixture B](20.0 mg, 0.03 mmol, 1.0 eq) in EtOAc (1 mL) was added 4N HCl in EtOAc (1.0 mL, 4.0 mmol, 130 eq) at 0° C. and the mixture was stirred at RT for 1 h. The mixture was concentrated under reduced pressure and the remaining residue was purified by chiral SFC to afford a white solid (4.5 mg) which was dissolved in EtOAc (1 ml). To this solution was added 4N HCl in EtOAc (1.0 mL, 4 mmol) and stirred for 1 h. The mixture was then concentrated under reduced pressure to afford the title compound (4.8 mg, 91% yield) as white solid, as hydrochloride salt. MS (ESI): 609.2 [M+H]⁺

### Example 16

### 1-[3-[(3R)-3-amino-8-fluoro-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,2,4-oxadiazol-5-yl]cyclopropanecarbonitrile

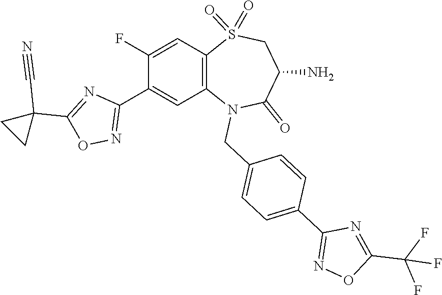 [SMILES: N#CC1(c2nc(-c3cc4c(cc3F)S(=O)(=O)C[C@H](N)C(=O)N4Cc3ccc(-c4noc(C(F)(F)F)n4)cc3)no2)CC1]

### Step a) [(Z)-[amino-[(3R)-3-(tert-butoxycarbonylamino)-8-fluoro-4-oxo-3,5-dihydro-2H-1,5-benzothiazepin-7-yl]methylene]amino]1-cyanocyclopropanecarboxylate

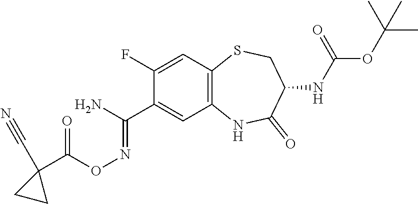 [SMILES: CC(C)(C)OC(=O)N[C@H]1CSc2cc(F)c(/C(N)=N/OC(=O)C3(C#N)CC3)cc2NC1=O]

The title compound was prepared in analogy to general procedure 8a from tert-butyl N-[(3R)-8-fluoro-7-[(Z)-N′-hydroxycarbamimidoyl]-4-oxo-3,5-dihydro-2H-1,5-benzothiazepin-3-yl]carbamate (Example 7, step d) (1 g, 2.7 mmol, 1.0 eq) and 1-cyano-1-cyclopropanecarboxylic acid (300 mg, 2.7 mmol, 1.0 eq, CAS: 6914-79-0) and was obtained as a white solid (650 mg, 35% yield). MS (ESI) 408.1 [M-isobutene+H]⁺

### Step b) tert-butyl N-[(3R)-7-[5-(I-cyanocyclopropyl)-1,2,4-oxadiazol-3-yl]-8-fluoro-4-oxo-3,5-dihydro-2H-1,5-benzothiazepin-3-yl]carbamate

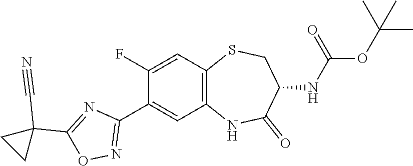 [SMILES: CC(C)(C)OC(=O)N[C@H]1CSc2cc(F)c(-c3noc(C4(C#N)CC4)n3)cc2NC1=O]

The title compound was prepared in analogy to general procedure 9a from [(Z)-[amino-[(3R)-3-(tert-butoxycarbonylamino)-8-fluoro-4-oxo-3,5-dihydro-2H-1,5-benzothiazepin-7-yl]methylene]amino]1-cyanocyclopropanecarboxylate (650 g, 1.4 mmol, 1.0) and was obtained as yellow solid (160 mg, 25% yield). MS (ESI) 390.1 [M-isobutene+H]⁺

### Step c) tert-butyl N-[(3R)-7-[5-(I-cyanocyclopropyl)-1,2,4-oxadiazol-3-yl]-8-fluoro-1,1,4-trioxo-3,5-dihydro-2H-1λ⁶,5-benzothiazepin-3-yl]carbamate

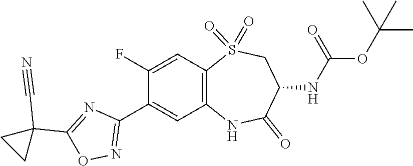 [SMILES: CC(C)(C)OC(=O)N[C@H]1CS(=O)(=O)c2cc(F)c(-c3noc(C4(C#N)CC4)n3)cc2NC1=O]

The title compound was prepared in analogy to general procedure 10 from tert-butyl N-[(3R)-7-[5-(1-cyanocyclopropyl)-1,2,4-oxadiazol-3-yl]-8-fluoro-4-oxo-3,5-dihydro-2H-1,5-benzothiazepin-3-yl]carbamate (160 mg, 0.36 mmol) and was obtained as yellow oil (140 mg, 60% yield). MS (ESI): 378.1 [M-Boc+H]⁺

### Step d) tert-butyl N-[(3R)-7-[5-(1-cyanocyclopropyl)-1,2,4-oxadiazol-3-yl]-8-fluoro-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate

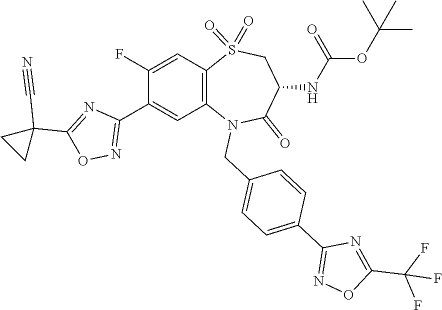 [SMILES: CC(C)(C)OC(=O)N[C@H]1CS(=O)(=O)c2cc(F)c(-c3noc(C4(C#N)CC4)n3)cc2N(Cc2ccc(-c3noc(C(F)(F)F)n3)cc2)C1=O]

The title compound was prepared in analogy to general procedure 1a from tert-butyl N-[(3R)-7-[5-(1-cyanocyclopropyl)-1,2,4-oxadiazol-3-yl]-8-fluoro-1,1,4-trioxo-3,5-dihydro-2H-1λ⁶,5-benzothiazepin-3-yl]carbamate (70 mg, 0.15 mmol, 1.0 eq) and 3-[4-(bromomethyl)phenyl]-5-(trifluoromethyl)-1,2,4-oxadiazole (49.5 mg, 0.2 mmol, 2 eq, CAS: 2093101-98-3) and was obtained as yellow oil (80 mg, 73% yield). MS (ESI): 604.2 [M-Boc+H]⁺

### Step e) 1-[3-[(3R)-3-amino-8-fluoro-1,1,4-trioxo-5-[[4-[5-(trifluoromethyl)-1,2,4-oxadiazol-3-yl]phenyl]methyl]-2,3-dihydro-1λ⁶, 5-benzothiazepin-7-yl]-1,2,4-oxadiazol-5-yl]cyclopropanecarbonitrile

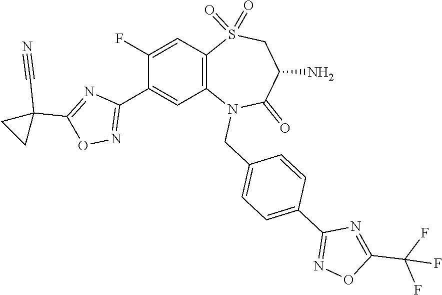 [SMILES: N#CC1(c2nc(-c3cc4c(cc3F)S(=O)(=O)C[C@H](N)C(=O)N4Cc3ccc(-c4noc(C(F)(F)F)n4)cc3)no2)CC1]

The title compound was prepared in analogy to general procedure 11c from tert-butyl N-[(3R)-7-[5-(1-cyanocyclopropyl)-1,2,4-oxadiazol-3-yl]-8-fluoro-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (80 mg, 0.11 mmol) and was obtained as white solid, as hydrochloride salt (24.9 mg, 34% yield). MS (ESI): 604.2 [M+H]⁺

### Example 17 of the Following Table was Prepared in Analogy to Example 16 Using the Appropriate Benzyl Bromide Building Block

| | | | | MS, ESI: |
|---|---|---|---|---|
| Ex. | Structure | Systematic Name | Building Blocks | m/z |
| 17 | 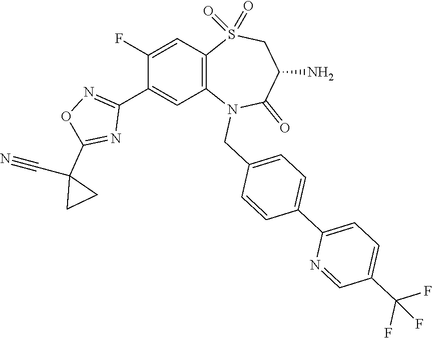 [SMILES: N#CC1(c2nc(-c3cc4c(cc3F)S(=O)(=O)C[C@H](N)C(=O)N4Cc3ccc(-c4ccc(C(F)(F)F)cn4)cc3)no2)CC1] | 1-[3-[(3R)-3-amino-8- fluoro-1,1,4-trioxo-5- [4-[5- (trifluoromethyl)-2- pyridyl]phenyl]methyl]- 2,3-dihydro-1λ⁶,5- benzothiazepin-7-yl]- 1,2,4-oxadiazol-5-yl] cyclopropanecarbonitrile (*) | 2-[4- (bromomethyl) phenyl]-5- (trifluoromethyl) pyridine) (CAS: 1056641-21-4) | 613.2 [M + H]⁺ |

(*) as hydrochloride salt

### Example 18

### (3R)-3-amino-8-fluoro-7-[5-[2-(hydroxymethyl)tetrahydrofuran-2-yl]-1,2,4-oxadiazol-3-yl]-1,1-dioxo-5-[[4-[5-(trifluoromethyl)-1,2,4-oxadiazol-3-yl]phenyl]methyl]-2,3-dihydro-1σ⁶,5-benzothiazepin-4-one

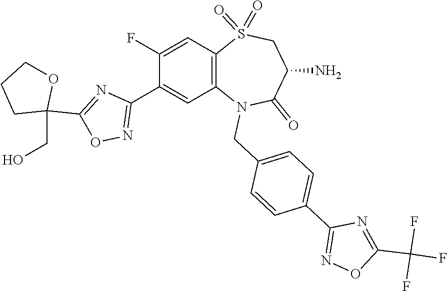 [SMILES: N[C@H]1CS(=O)(=O)c2cc(F)c(-c3noc(C4(CO)CCCO4)n3)cc2N(Cc2ccc(-c3noc(C(F)(F)F)n3)cc2)C1=O]

### Step a) tert-butyl N-[(3R)-7-cyano-8-fluoro-4-oxo-5-[[4-[5-(trifluoromethyl)-1,2,4-oxadiazol-3-yl]phenyl]methyl]-2,3-dihydro-1,5-benzothiazepin-3-yl]carbamate

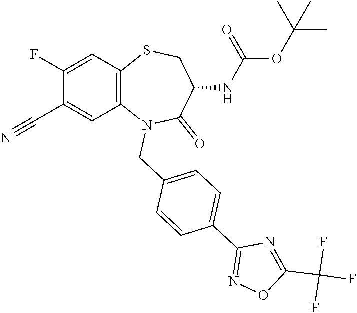 [SMILES: CC(C)(C)OC(=O)N[C@H]1CSc2cc(F)c(C#N)cc2N(Cc2ccc(-c3noc(C(F)(F)F)n3)cc2)C1=O]

The title compound was prepared in analogy to general procedure 1a tert-butyl N-[(3R)-7-cyano-8-fluoro-4-oxo-3,5-dihydro-2H-1,5-benzothiazepin-3-yl]carbamate (Example 104 step c) (150 mg, 0.445 mmol) and 3-[4-(bromomethyl)phenyl]-5-(trifluoromethyl)-1,2,4-oxadiazole (CAS 2093101-98-3) (204 mg, 0.67 mmol) and was obtained as a white solid (216 mg, 74% yield). MS (ESI): 508.2 [M-isobutene+H]⁺.

### Step b) tert-butyl N-[(3R)-8-fluoro-7-[(Z)-N′-hydroxycarbamimidoyl]-4-oxo-5-[[4-[5-(trifluoromethyl)-1,2,4-oxadiazol-3-yl]phenyl]methyl]-2,3-dihydro-1,5-benzothiazepin-3-yl]carbamate

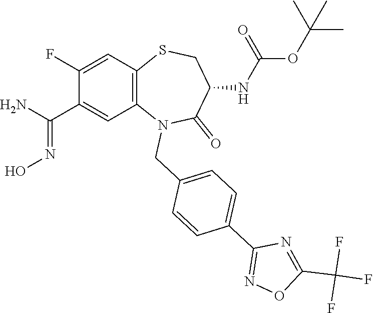 [SMILES: CC(C)(C)OC(=O)N[C@H]1CSc2cc(F)c(/C(N)=N/O)cc2N(Cc2ccc(-c3noc(C(F)(F)F)n3)cc2)C1=O]

The title compound was prepared in analogy to general procedure 6 from tert-butyl N-[(3R)-7-cyano-8-fluoro-4-oxo-5-[[4-[5-(trifluoromethyl)-1,2,4-oxadiazol-3-yl]phenyl]methyl]-2,3-dihydro-1,5-benzothiazepin-3-yl]carbamate (200 mg, 0.302 mmol) and was obtained as a white solid (200 mg, 94% yield). MS (ESI): 597.4 [M+H]⁺.

### Step c) [(Z)-[amino-[(3R)-3-(tert-butoxycarbonylamino)-8-fluoro-4-oxo-5-[[4-[5-(trifluoromethyl)-1,2,4-oxadiazol-3-yl]phenyl]methyl]-2,3-dihydro-1,5-benzothiazepin-7-yl]methylene]amino]2-(hydroxymethyl)tetrahydrofuran-2-carboxylate

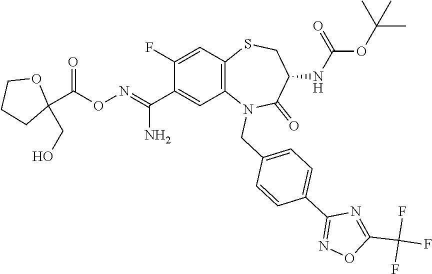 [SMILES: CC(C)(C)OC(=O)N[C@H]1CSc2cc(F)c(/C(N)=N/OC(=O)C3(CO)CCCO3)cc2N(Cc2ccc(-c3noc(C(F)(F)F)n3)cc2)C1=O]

The title compound was prepared in analogy to general procedure 8a from tert-butyl N-[(3R)-8-fluoro-7-[(Z)-N′-hydroxycarbamimidoyl]-4-oxo-5-[[4-[5-(trifluoromethyl)-1,2,4-oxadiazol-3-yl]phenyl]methyl]-2,3-dihydro-1,5-benzothiazepin-3-yl]carbamate (100 mg, 0.142 mmol) and 2-(hydroxymethyl)tetrahydrofuran-2-carboxylic acid (CAS 442877-01-2) (25 mg, 0.17 mmol) and was obtained as an white powder (72.7 mg, 53% yield). MS (ESI): 723.5 [M−H]

### Step d) tert-butyl N-[(3R)-8-fluoro-7-[5-[2-(hydroxymethyl)tetrahydrofuran-2-yl]-1,2,4-oxadiazol-3-yl]-4-oxo-5-[[4-[5-(trifluoromethyl)-1,2,4-oxadiazol-3-yl]phenyl]methyl]-2,3-dihydro-1,5-benzothiazepin-3-yl]carbamate

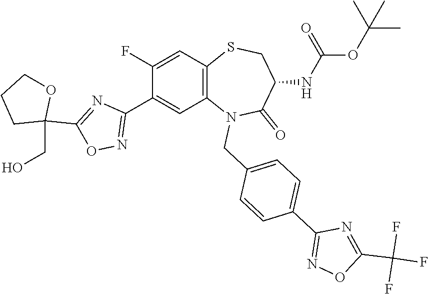 [SMILES: CC(C)(C)OC(=O)N[C@H]1CSc2cc(F)c(-c3noc(C4(CO)CCCO4)n3)cc2N(Cc2ccc(-c3noc(C(F)(F)F)n3)cc2)C1=O]

The title compound was prepared in analogy to general procedure 9a from [(Z)-[amino-[(3R)-3-(tert-butoxycarbonylamino)-8-fluoro-4-oxo-5-[[4-[5-(trifluoromethyl)-1,2,4-oxadiazol-3-yl]phenyl]methyl]-2,3-dihydro-1,5-benzothiazepin-7-yl]methylene]amino]2-(hydroxymethyl)tetrahydrofuran-2-carboxylate (72.7 mg, 0.075 mmol) and was obtained as a white solid (50.3 mg, 94% yield). MS (ESI): 651.2 [M-isobutene+H]⁺.

### Step e) tert-butyl N-[(3R)-8-fluoro-7-[5-[2-(hydroxymethyl)tetrahydrofuran-2-yl]-1,2,4-oxadiazol-3-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate

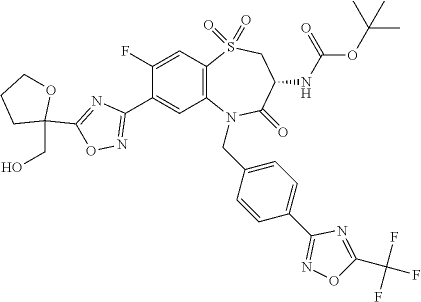 [SMILES: CC(C)(C)OC(=O)N[C@H]1CS(=O)(=O)c2cc(F)c(-c3noc(C4(CO)CCCO4)n3)cc2N(Cc2ccc(-c3noc(C(F)(F)F)n3)cc2)C1=O]

The title compound was prepared in analogy to general procedure 10 from tert-butyl N-[(3R)-8-fluoro-7-[5-[2-(hydroxymethyl)tetrahydrofuran-2-yl]-1,2,4-oxadiazol-3-yl]-4-oxo-5-[[4-[5-(trifluoromethyl)-1,2,4-oxadiazol-3-yl]phenyl]methyl]-2,3-dihydro-1,5-benzothiazepin-3-yl]carbamate (50.3 mg, 0.071 mmol) and was obtained as a white powder (23.7 mg, 45% yield). MS (ESI): 683.2 [M-isobutene+H]⁺.

### Step e) (3R)-3-amino-8-fluoro-7-[5-[2-(hydroxymethyl)tetrahydrofuran-2-yl]-1,2,4-oxadiazol-3-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

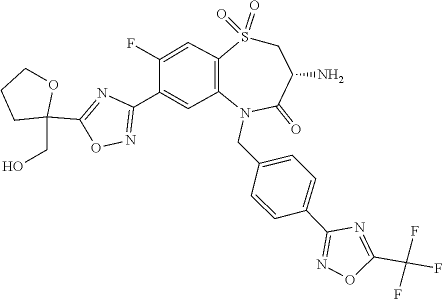 [SMILES: N[C@H]1CS(=O)(=O)c2cc(F)c(-c3noc(C4(CO)CCCO4)n3)cc2N(Cc2ccc(-c3noc(C(F)(F)F)n3)cc2)C1=O]

The title compound was prepared in analogy to general procedure 11b from tert-butyl N-[(3R)-8-fluoro-7-[5-[2-(hydroxymethyl)tetrahydrofuran-2-yl]-1,2,4-oxadiazol-3-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (23.7 mg, 0.032 mmol) and was obtained as an off-white powder, as a hydrochloride salt (20.9 mg, 96% yield). MS (ESI): 639.2 [M+H]⁺.

### Example 19

### (3R)-3-amino-7-(5-tert-butyl-1,3,4-oxadiazol-2-yl)-5-[[4-(5-tert-butyl-1,3,4-oxadiazol-2-yl)phenyl]methyl]-1,1-dioxo-2,3-dihydro-1λ⁶,5-benzothiazepin-4-one

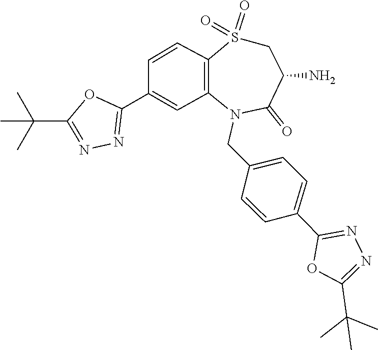 [SMILES: CC(C)(C)c1nnc(-c2ccc(CN3C(=O)[C@@H](N)CS(=O)(=O)c4ccc(-c5nnc(C(C)(C)C)o5)cc43)cc2)o1]

### Step a) (3R)-3-(tert-butoxycarbonylamino)-4-oxo-3,5-dihydro-2H-1,5-benzothiazepine-7-carboxylic acid

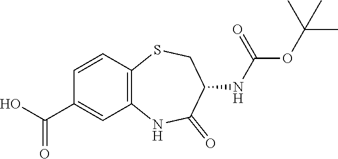 [SMILES: CC(C)(C)OC(=O)N[C@H]1CSc2ccc(C(=O)O)cc2NC1=O]

The title compound was prepared from methyl (3R)-3-(tert-butoxycarbonylamino)-4-oxo-3,5-dihydro-2H-1,5-benzothiazepine-7-carboxylate (2.5 g, 7.09 mmol, CAS 2089150-62-7) in analogy to general procedure 2 and was obtained as orange amorphous solid (2.26 g, 89%). MS (ESI): 283.0 [M-isobutene+H]⁺

### Step b) tert-butyl N-[(3R)-7-(hydrazinecarbonyl)-4-oxo-3,5-dihydro-2H-1,5-benzothiazepin-3-yl]carbamate

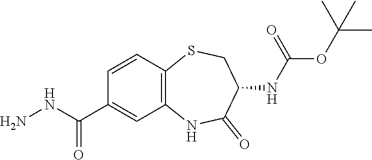 [SMILES: CC(C)(C)OC(=O)N[C@H]1CSc2ccc(C(=O)NN)cc2NC1=O]

The title compound was prepared from (3R)-3-(tert-butoxycarbonylamino)-4-oxo-3,5-dihydro-2H-1,5-benzothiazepine-7-carboxylic acid (1120 mg, 3.31 mmol) in analogy to general procedure 3 and was obtained as light yellow solid (1036 mg, 80%). MS (ESI): 351.2 [M−H]

### Step c) tert-butyl N-[(3R)-7-[(2,2-dimethylpropanoylamino)carbamoyl]-4-oxo-3,5-dihydro-2H-1,5-benzothiazepin-3-yl]carbamate

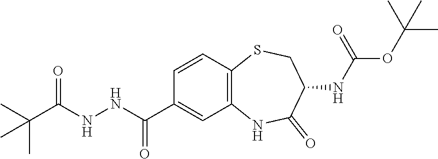 [SMILES: CC(C)(C)OC(=O)N[C@H]1CSc2ccc(C(=O)NNC(=O)C(C)(C)C)cc2NC1=O]

The title compound was prepared from tert-butyl N-[(3R)-7-(hydrazinecarbonyl)-4-oxo-3,5-dihydro-2H-1,5-benzothiazepin-3-yl]carbamate (4.5 g, 7.39 mmol, 1 eq) and pivalic acid (889.0 mg, 8.7 mmol, 1.18 eq) in analogy to general procedure 4a and was obtained as light yellow solid (2.95 g, 6.76 mmol, 84% yield). MS (ESI): 381.1 [M+H-isobutene]⁺

### Step d) tert-butyl N-[(3R)-7-(5-tert-butyl-1,3,4-oxadiazol-2-yl)-4-oxo-3,5-dihydro-2H-1λ⁶,5-benzothiazepin-3-yl]carbamate

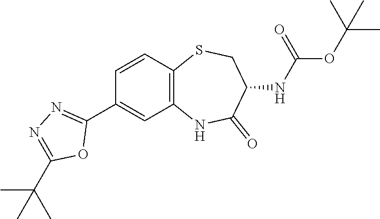 [SMILES: CC(C)(C)OC(=O)N[C@H]1CSc2ccc(-c3nnc(C(C)(C)C)o3)cc2NC1=O]

The title compound was prepared from tert-butyl N-[(3R)-7-[(2,2-dimethylpropanoylamino)carbamoyl]-4-oxo-3,5-dihydro-2H-1,5-benzothiazepin-3-yl]carbamate (2.9 g, 6.64 mmol) in analogy to general procedure 5a and was obtained as light yellow solid (1.6 g, 3.82 mmol, 54% yield). MS (ESI): 363.1 [M+H-isobutene]⁺

### Step e) tert-butyl N-[(3R)-7-(5-tert-butyl-1,3,4-oxadiazol-2-yl)-1,1,4-trioxo-3,5-dihydro-2H-1λ⁶,5-benzothiazepin-3-yl]carbamate

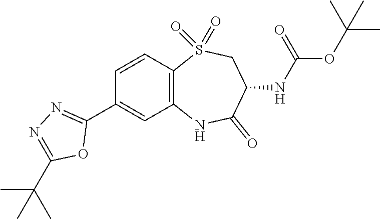 [SMILES: CC(C)(C)OC(=O)N[C@H]1CS(=O)(=O)c2ccc(-c3nnc(C(C)(C)C)o3)cc2NC1=O]

The title compound was prepared from tert-butyl N-[(3R)-7-(5-tert-butyl-1,3,4-oxadiazol-2-yl)-4-oxo-3,5-dihydro-2H-1,5-benzothiazepin-3-yl]carbamate (241 mg, 0.58 mmol) in analogy to general procedure 10 and was obtained as white solid (270 mg, 0.6 mmol, 94.5% yield). MS (ESI): 395.2 [M+H-isobutene]⁺

### Step f) tert-butyl N-[(3R)-7-(5-tert-butyl-1,3,4-oxadiazol-2-yl)-5-[[4-(5-tert-butyl-1,3,4-oxadiazol-2-yl)phenyl]methyl]-1,1,4-trioxo-2,3-dihydro-1λ⁶,5-benzothiazepin-3-yl]carbamate

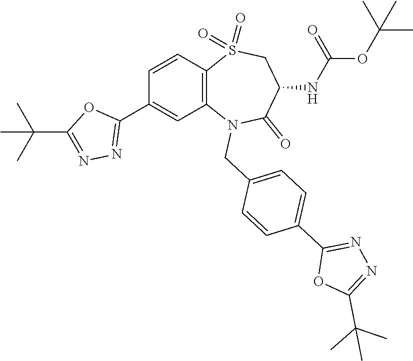 [SMILES: CC(C)(C)OC(=O)N[C@H]1CS(=O)(=O)c2ccc(-c3nnc(C(C)(C)C)o3)cc2N(Cc2ccc(-c3nnc(C(C)(C)C)o3)cc2)C1=O]

The title compound was prepared from tert-butyl N-[(3R)-7-(5-tert-butyl-1,3,4-oxadiazol-2-yl)-1,1,4-trioxo-3,5-dihydro-2H-1λ⁶,5-benzothiazepin-3-yl]carbamate (100 mg, 0.22 mmol, 1.0 eq) and Intermediate 3 (78.94 mg, 0.22 mmol, 1.0 eq) in analogy to general procedure 1a and was obtained as light yellow solid (150 mg, 0.23 mmol, 77% yield). MS (ESI): 665.3 [M+H]⁺

### Step g) (3R)-3-amino-7-(5-tert-butyl-1,3,4-oxadiazol-2-yl)-5-[[4-(5-tert-butyl-1,3,4-oxadiazol-2-yl)phenyl]methyl]-1,1-dioxo-2,3-dihydro-1λ⁶,5-benzothiazepin-4-one

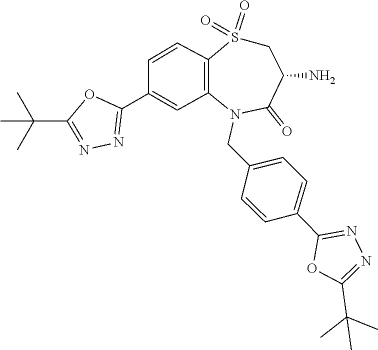 [SMILES: CC(C)(C)c1nnc(-c2ccc(CN3C(=O)[C@@H](N)CS(=O)(=O)c4ccc(-c5nnc(C(C)(C)C)o5)cc43)cc2)o1]

The title compound was prepared from tert-butyl N-[(3R)-7-(5-tert-butyl-1,3,4-oxadiazol-2-yl)-5-[[4-(5-tert-butyl-1,3,4-oxadiazol-2-yl)phenyl]methyl]-1,1,4-trioxo-2,3-dihydro-1λ⁶,5-benzothiazepin-3-yl]carbamate (150 mg, 0.23 mmol) in analogy to general procedure 11c and was obtained as yellow solid, as hydrochloride salt (23.4 mg, 0.04 mmol, 17% yield). MS (ESI): 565.2 [M+H]⁺

The examples of the following table were prepared in analogy to Example 19, using the appropriate benzyl bromide building block.

| | | | | MS,ESI: |
|---|---|---|---|---|
| Ex. | Structure | Systematic Name | Building Blocks | m/z |
| 20 | 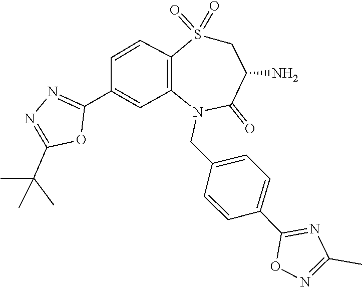 [SMILES: Cc1noc(-c2ccc(CN3C(=O)[C@@H](N)CS(=O)(=O)c4ccc(-c5nnc(C(C)(C)C)o5)cc43)cc2)n1] | (3R)-3-amino-7-(5-tert- butyl-1,3,4-oxadiazol-2- yl)-5-[4-(3-methyl- 1,2,4-oxadiazol-5- yl)phenyl]methyl]-1,1- dioxo-2,3-dihydro- 1λ⁶,5-benzothiazepin-4- one | 5-[4- (bromomethyl)phenyl]- 3-methyl-1,2,4- oxadiazole (CAS 362529-03-1) | 523.1 [M + H]⁺ |
| 21 | 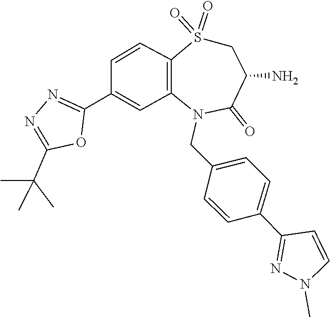 [SMILES: Cn1ccc(-c2ccc(CN3C(=O)[C@@H](N)CS(=O)(=O)c4ccc(-c5nnc(C(C)(C)C)o5)cc43)cc2)n1] | (3R)-3-amino-7-(5-tert- butyl-1,3,4-oxadiazol-2- yl)-5-[[4-(1- methylpyrazol-3- yl)phenyl]methyl]-1,1- dioxo-2,3-dihydro- 1λ⁶,5-benzothiazepin-4- one | 3-[4- (bromomethyl)phenyl]- 1-methyl-1H- pyrazole (CAS 1820815-38-0) | 521.1 [M + H]⁺ |
| 22 | 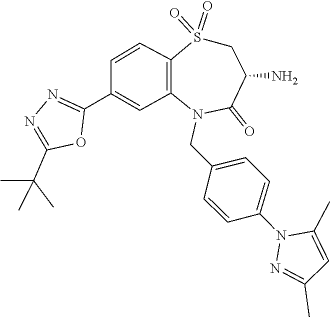 [SMILES: Cc1cc(C)n(-c2ccc(CN3C(=O)[C@@H](N)CS(=O)(=O)c4ccc(-c5nnc(C(C)(C)C)o5)cc43)cc2)n1] | (3R)-3-amino-7-(5-tert- butyl-1,3,4-oxadiazol-2- yl)-5-[4-(3,5- dimethylpyrazol-1- yl)phenyl]methyl]-1,1- dioxo-2,3-dihydro- 1λ⁶,5-benzothiazepin-4- one | 1-[4- (bromomethyl)phenyl]- 3,5-diemthyl-1H- pyrazole (CAS 937796-05-9) | 535.1 [M + H]⁺ |
| 23 | 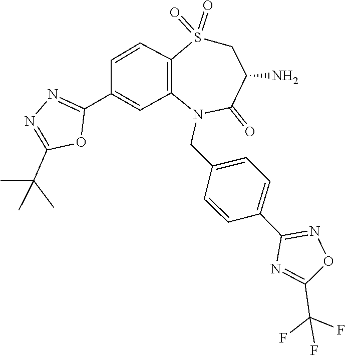 [SMILES: CC(C)(C)c1nnc(-c2ccc3c(c2)N(Cc2ccc(-c4noc(C(F)(F)F)n4)cc2)C(=O)[C@@H](N)CS3(=O)=O)o1] | (3R)-3-amino-7-(5-tert- butyl-1,3,4-oxadiazol-2- yl)-1,1-dioxo-5-[[4-[5- (trifluoromethyl)-1,2,4- oxadiazol-3- yl]phenyl]methyl]-2,3- dihydro-1λ⁶,5- benzothiazepin-4-one | Intermediate 4 | 577.1 [M + H]⁺ |
| 24 | 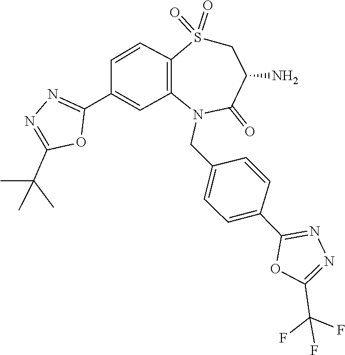 [SMILES: CC(C)(C)c1nnc(-c2ccc3c(c2)N(Cc2ccc(-c4nnc(C(F)(F)F)o4)cc2)C(=O)[C@@H](N)CS3(=O)=O)o1] | (3R)-3-amino-7-(5-tert- butyl-1,3,4-oxadiazol-2- yl)-1, 1-dioxo-5-[[4-[5- (trifluoromethyl)-1,3,4- oxadiazol-2- yl]phenyl]methyl]-2,3- dihydro-1λ⁶,5- benzothiazepin-4-one (*) | 3-[4- (bromomethyl)phenyl]- 5-(trifluoromethyl)- 1,2,4-oxadiazole (CAS 2093101-98-3) | 577.2 [M + H]⁺ |
| 25 | 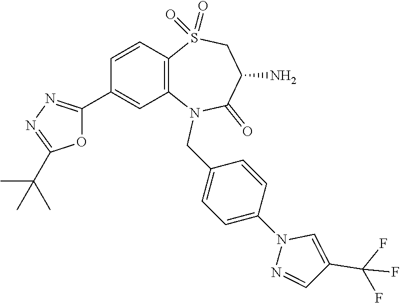 [SMILES: CC(C)(C)c1nnc(-c2ccc3c(c2)N(Cc2ccc(-n4cc(C(F)(F)F)cn4)cc2)C(=O)[C@@H](N)CS3(=O)=O)o1] | (3R)-3-amino-7-(5-tert- butyl-1,3,4-oxadiazol-2- yl)-1,1-dioxo-5-[[4-[4- (trifluoromethyl)pyrazol- 1-yl]phenyl]methyl]- 2,3-dihydro-1λ⁶,5- benzothiazepin-4-one (*) | 1-[4- (bromomethyl)phenyl]- 4-(trifluoromethyl)- 1H-pyrazole (CAS 1928384-61-5) | 575.2 [M + H]⁺ |
| 26 | 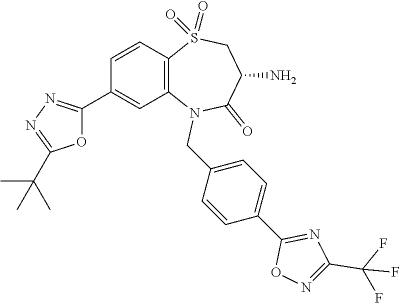 [SMILES: CC(C)(C)c1nnc(-c2ccc3c(c2)N(Cc2ccc(-c4nc(C(F)(F)F)no4)cc2)C(=O)[C@@H](N)CS3(=O)=O)o1] | (3R)-3-amino-7-(5-tert- butyl-1,3,4-oxadiazol-2- yl)-1, 1-dioxo-5-[[4-[3- (trifluoromethyl)-1,2,4- oxadiazol-5- yl]phenyl]methyl]-2,3- dihydro-1λ⁶,5- benzothiazepin-4-one | Intermediate 7 | 577.2 [M + H]⁺ |
| 27 | 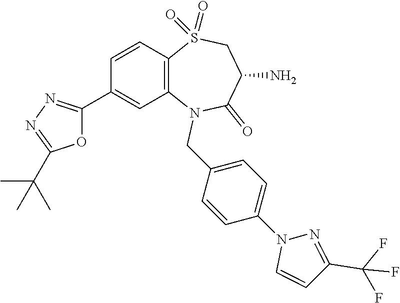 [SMILES: CC(C)(C)c1nnc(-c2ccc3c(c2)N(Cc2ccc(-n4ccc(C(F)(F)F)n4)cc2)C(=O)[C@@H](N)CS3(=O)=O)o1] | (3R)-3-amino-7-(5-tert- butyl-1,3,4-oxadiazol-2- yl)-1,1-dioxo-5-[[4-[3- (trifluoromethyl)pyrazol- 1-yl]phenyl]methyl]- 2,3-dihydro-1λ⁶,5- benzothiazepin-4-one (*) | 1-[4- (Chloromethyl)phenyl]- 1-3-(trifluoromethyl)- 1H-pyrazole (CAS 1284983-51-2) | 575.2 [M + H]⁺ |
| 28 | 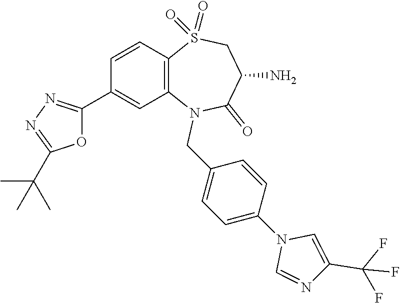 [SMILES: CC(C)(C)c1nnc(-c2ccc3c(c2)N(Cc2ccc(-n4cnc(C(F)(F)F)c4)cc2)C(=O)[C@@H](N)CS3(=O)=O)o1] | (3R)-3-amino-7-(5-tert- butyl-1,3,4-oxadiazol-2- yl)-1,1-dioxo-5-[[4-[4- (trifluoromethyl)imidazol- 1-yl]phenyl]methyl]- 2,3-dihydro-1λ⁶,5- benzothiazepin-4-one (*) | Intermediate 8 | 575.2 [M + H]⁺ |
| 29 | 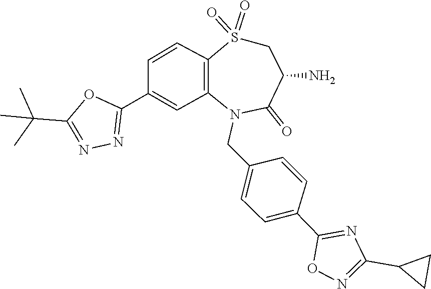 [SMILES: CC(C)(C)c1nnc(-c2ccc3c(c2)N(Cc2ccc(-c4nc(C5CC5)no4)cc2)C(=O)[C@@H](N)CS3(=O)=O)o1] | (3R)-3-amino-7-(5-tert- butyl-1,3,4-oxadiazol-2- yl)-5-[4-(3- cyclopropyl-1,2,4- oxadiazol-5- yl)phenyl]methyl]-1,1- dioxo-2,3-dihydro- 1λ⁶,5-benzothiazepin-4- one | Intermediate 5 | 549.3 [M + H]⁺ |
| 30 | 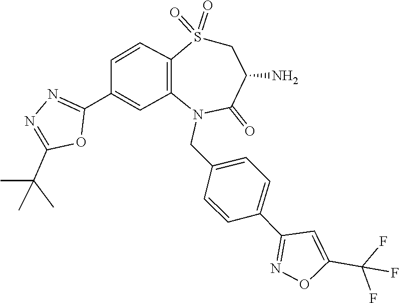 [SMILES: CC(C)(C)c1nnc(-c2ccc3c(c2)N(Cc2ccc(-c4cc(C(F)(F)F)on4)cc2)C(=O)[C@@H](N)CS3(=O)=O)o1] | (3R)-3-amino-7-(5-tert- butyl-1,3,4-oxadiazol-2- yl)-1,1-dioxo-5-[[4-[5- (trifluoromethyl)isoxazo 1-3-yl]phenyl]methyl]- 2,3-dihydro-1λ⁶,5- benzothiazepin-4-one (*) | Intermediate 6 | 576.4 [M + H]⁺ |
| 31 | 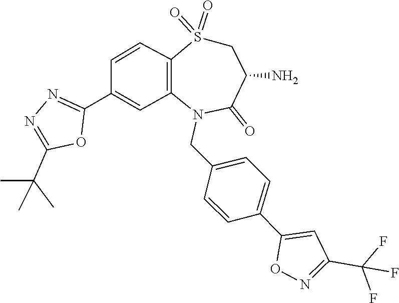 [SMILES: CC(C)(C)c1nnc(-c2ccc3c(c2)N(Cc2ccc(-c4cc(C(F)(F)F)no4)cc2)C(=O)[C@@H](N)CS3(=O)=O)o1] | (3R)-3-amino-7-(5-tert- butyl-1,3,4-oxadiazol-2- yl)-1,1-dioxo-5-[[4-[3- (trifluoromethyl)isoxazo 1-5-yl]phenyl]methyl]- 2,3-dihydro-1λ⁶,5- benzothiazepin-4-one | Intermediate 9 | 576.4 [M + H]⁺ |
| 89 | 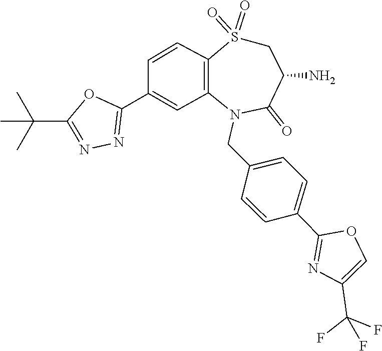 [SMILES: CC(C)(C)c1nnc(-c2ccc3c(c2)N(Cc2ccc(-c4nc(C(F)(F)F)co4)cc2)C(=O)[C@@H](N)CS3(=O)=O)o1] | (3R)-3-amino-7-(5-tert- butyl-1,3,4-oxadiazol-2- yl)-1,1-dioxo-5-[[4-[4- (trifluoromethyl)oxazol- 2-yl]phenyl]methyl]- 2,3-dihydro-1λ⁶,5- benzothiazepin-4-one | Intermediate 37 | 576.3 [M + H]⁺ |
| 90 | 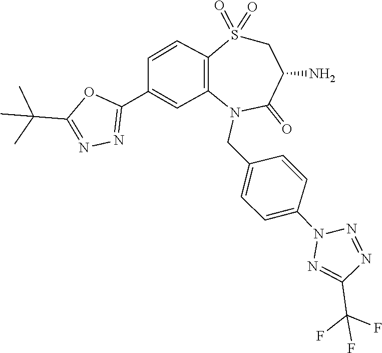 [SMILES: CC(C)(C)c1nnc(-c2ccc3c(c2)N(Cc2ccc(-n4nnc(C(F)(F)F)n4)cc2)C(=O)[C@@H](N)CS3(=O)=O)o1] | (3R)-3-amino-7-(5-tert- butyl-1,3,4-oxadiazol-2- yl)-1,1-dioxo-5-[[4-[5- (trifluoromethyl)tetrazol- 2-yl]phenyl]methyl]- 2,3-dihydro-1λ⁶,5- benzothiazepin-4-one | Intermediate 35 | 577.3 [M + H]⁺ |
| 91 | 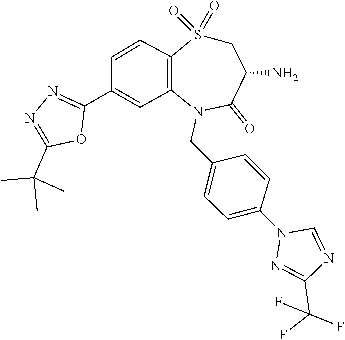 [SMILES: CC(C)(C)c1nnc(-c2ccc3c(c2)N(Cc2ccc(-n4cnc(C(F)(F)F)n4)cc2)C(=O)[C@@H](N)CS3(=O)=O)o1] | (3R)-3-amino-7-(5-tert- butyl-1,3,4-oxadiazol-2- yl)-1, 1-dioxo-5-[[4-[3- (trifluoromethyl)-1,2,4- triazol-1- yl]phenyl]methyl]-2,3- dihydro-1λ⁶,5- benzothiazepin-4-one (*) | Intermediate 22 | 576.3 [M + H]⁺ |
| 92 | 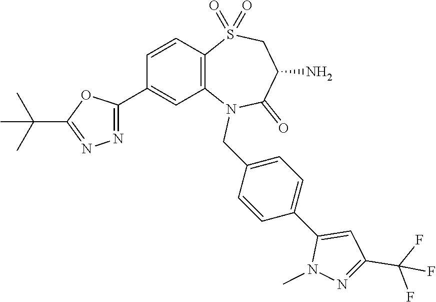 [SMILES: Cn1nc(C(F)(F)F)cc1-c1ccc(CN2C(=O)[C@@H](N)CS(=O)(=O)c3ccc(-c4nnc(C(C)(C)C)o4)cc32)cc1] | (3R)-3-amino-7-(5-tert- butyl-1,3,4-oxadiazol-2- yl)-5-[4-[2-methyl-5- (trifluoromethyl)pyrazol- 3-yl]phenyl]methyl]- 1,1-dioxo-2,3-dihydro- 1λ⁶,5-benzothiazepin-4- one (*) | Intermediate 24 | 589.2 [M + H]⁺ |
| 93 | 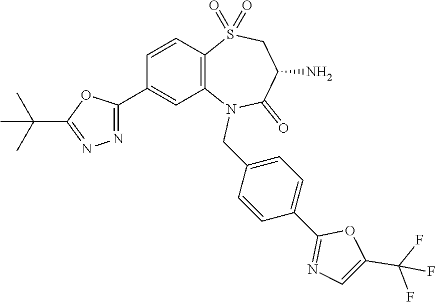 [SMILES: CC(C)(C)c1nnc(-c2ccc3c(c2)N(Cc2ccc(-c4ncc(C(F)(F)F)o4)cc2)C(=O)[C@@H](N)CS3(=O)=O)o1] | (3R)-3-amino-7-(5-tert- butyl-1,3,4-oxadiazol-2- yl)-1, 1-dioxo-5-[[4-[5- (trifluoromethyl)oxazol- 2-yl]phenyl]methyl]- 2,3-dihydro-1λ⁶,5- benzothiazepin-4-one (*) | Intermediate 23 | 576.3 [M + H]⁺ |

(*) as hydrochloride salt

### Example 32

### (3R)-3-amino-7-[5-(1-amino-2,2,2-trifluoro-1-methyl-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

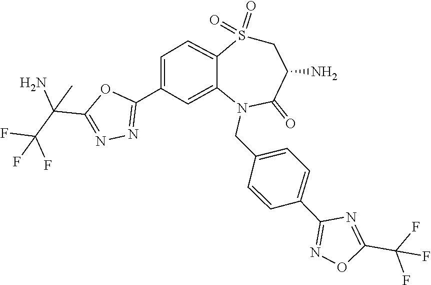 [SMILES: CC(N)(c1nnc(-c2ccc3c(c2)N(Cc2ccc(-c4noc(C(F)(F)F)n4)cc2)C(=O)[C@@H](N)CS3(=O)=O)o1)C(F)(F)F]

### Step a) (3R)-3-(tert-butoxycarbonylamino)-1,1,4-trioxo-3,5-dihydro-2H-1λ⁶, 5-benzothiazepine-7-carboxylic acid

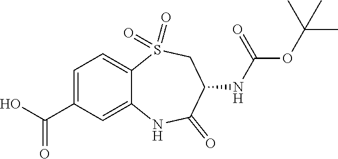 [SMILES: CC(C)(C)OC(=O)N[C@H]1CS(=O)(=O)c2ccc(C(=O)O)cc2NC1=O]

NaIO₄ (2.28 g, 10.66 mmol, 2.283 eq) was dissolved in water (22.37 mL) and cooled to 0° C. under inert atmosphere. RuCl₃ 3H₂O (12.21 mg, 46.69 μmol, 0.01 eq) was added and the reaction stirred for min. Then, a solution of (3R)-3-(tert-butoxycarbonylamino)-4-oxo-3,5-dihydro-2H-1,5-benzothiazepine-7-carboxylic acid (1580 mg, 4.67 mmol, 1.0 eq, Example 19, step a) in acetonitrile (25 mL) was added and the resulting grey suspension stirred for 3 h. The reaction was quenched by addition of isopropanol (2 ml) and then diluted with EtOAc and 2M HCl. The biphasic mixture was filtered through a plug of celite. The resulting filtrate was transferred to a separating funnel and the phases separated. The organic phase was washed with water, dried over anhydrous sodium sulfate and concentrated afford (3R)-3-(tert-butoxycarbonylamino)-1,1,4-trioxo-3,5-dihydro-2H-1λ⁶,5-benzothiazepine-7-carboxylic acid (1287 mg, 74%) as light yellow solid. MS (ESI): 369.1 [M−H]

### Step b) tert-butyl N-[(3R)-7-(hydrazinecarbonyl)-1,1,4-trioxo-3,5-dihydro-2H-1λ⁶,5-benzothiazepin-3-yl]carbamate

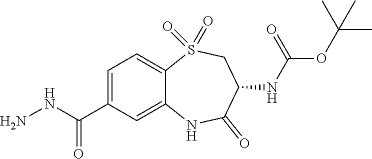 [SMILES: CC(C)(C)OC(=O)N[C@H]1CS(=O)(=O)c2ccc(C(=O)NN)cc2NC1=O]

The title compound was prepared from (3R)-3-(tert-butoxycarbonylamino)-1,1,4-trioxo-3,5-dihydro-2H-1λ⁶,5-benzothiazepine-7-carboxylic acid (500 mg, 877.48 μmol, 1 eq) in analogy to general procedure 3 and was obtained as yellow solid (282 mg, 72%). MS (ESI): 383.2 [M+H]⁺

### Step c) tert-butyl N-[(3R)-7-[[[2-(tert-butoxycarbonylamino)-3,3,3-trifluoro-2-methyl-propanoyl]amino]carbamoyl]-1,1,4-trioxo-3,5-dihydro-2H-1λ⁶,5-benzothiazepin-3-yl]carbamate

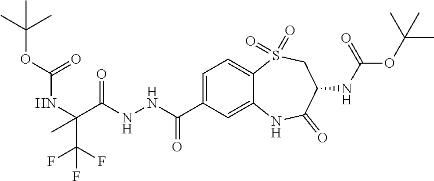 [SMILES: CC(C)(C)OC(=O)N[C@H]1CS(=O)(=O)c2ccc(C(=O)NNC(=O)C(C)(NC(=O)OC(C)(C)C)C(F)(F)F)cc2NC1=O]

The title compound was prepared from tert-butyl N-[(3R)-7-(hydrazinecarbonyl)-1,1,4-trioxo-3,5-dihydro-2H-1λ⁶,5-benzothiazepin-3-yl]carbamate (94 mg, 210.3 mol) and 2-(tert-butoxycarbonylamino)-3,3,3-trifluoro-2-methyl-propionic acid (64.91 mg, 252.36 μmol, 1.2 eq, CAS 170462-68-7) in analogy to general procedure 4a and was obtained as white powder (63.3 mg, 45%). MS (ESI): 622.2 [M+H]⁺

### Step d) tert-butyl N-[(3R)-7-[5-[1-(tert-butoxycarbonylamino)-2,2,2-trifluoro-1-methyl-ethyl]-1,3,4-oxadiazol-2-yl]-1,1,4-trioxo-3,5-dihydro-2H-1λ⁶,5-benzothiazepin-3-yl]carbamate

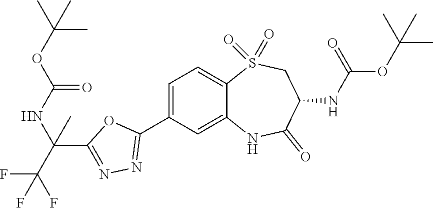 [SMILES: CC(C)(C)OC(=O)N[C@H]1CS(=O)(=O)c2ccc(-c3nnc(C(C)(NC(=O)OC(C)(C)C)C(F)(F)F)o3)cc2NC1=O]

The title compound was prepared from tert-butyl N-[(3R)-7-[[[2-(tert-butoxycarbonylamino)-3,3,3-trifluoro-2-methyl-propanoyl]amino]carbamoyl]-1,1,4-trioxo-3,5-dihydro-2H-1λ⁶,5-benzothiazepin-3-yl]carbamate (60 mg, 0.106 mmol, 1 eq) in analogy to general procedure 5b and was obtained as white solid (50.9 mg, 83%). MS (ESI): 604.2 [M+H]⁺

### Step e) tert-butyl N-[(3R)-7-[5-[1]-(tert-butoxycarbonylamino)-2,2,2-trifluoro-1-methyl-ethyl]-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate

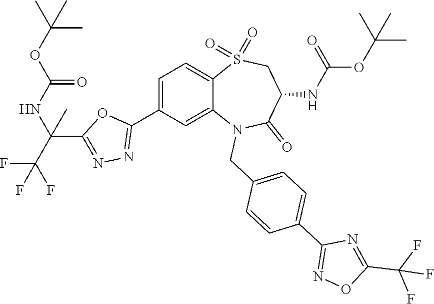 [SMILES: CC(C)(C)OC(=O)N[C@H]1CS(=O)(=O)c2ccc(-c3nnc(C(C)(NC(=O)OC(C)(C)C)C(F)(F)F)o3)cc2N(Cc2ccc(-c3noc(C(F)(F)F)n3)cc2)C1=O]

The title compound was prepared from tert-butyl N-[(3R)-7-[5-[1-(tert-butoxycarbonylamino)-2,2,2-trifluoro-1-methyl-ethyl]-1,3,4-oxadiazol-2-yl]-1,1,4-trioxo-3,5-dihydro-2H-1λ⁶,5-benzothiazepin-3-yl]carbamate (75.3 mg, 0.124 mmol, 1.0 eq) and 3-[4-(bromomethyl)phenyl]-5-(trifluoromethyl)-1,2,4-oxadiazole (57.27 mg, 0.187 mmol, 1.5 eq, CAS 2093101-98-3) in analogy to general procedure 1a and was obtained as white solid (73.5 mg, 71%). MS (ESI): 830.4 [M−H]

### Step f) (3R)-3-amino-7-[5-(1-amino-2,2,2-trifluoro-]-methyl-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one

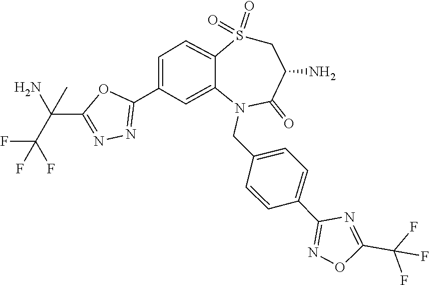 [SMILES: CC(N)(c1nnc(-c2ccc3c(c2)N(Cc2ccc(-c4noc(C(F)(F)F)n4)cc2)C(=O)[C@@H](N)CS3(=O)=O)o1)C(F)(F)F]

The title compound was prepared from tert-butyl N-[(3R)-7-[5-[1-(tert-butoxycarbonylamino)-2,2,2-trifluoro-1-methyl-ethyl]-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (75.3 mg, 0.091 mmol) in analogy to general procedure 11a and was obtained as white solid, as hydrochloride salt (53.8 mg, 89%). MS (ESI): 632.4 [M+H]⁺

The examples of the following table were prepared in analogy to Example 32, using the appropriate benzyl bromide building block.

| | | | Building | MS, |
|---|---|---|---|---|
| Ex. | Structure | Systematic Name | Block | ESI: m/z |
| 33 | 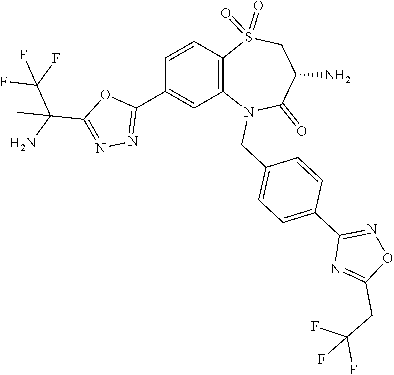 [SMILES: CC(N)(c1nnc(-c2ccc3c(c2)N(Cc2ccc(-c4noc(CC(F)(F)F)n4)cc2)C(=O)[C@@H](N)CS3(=O)=O)o1)C(F)(F)F] | (3R)-3-amino-7-[5-(1- amino-2,2,2-trifluoro-1- methyl-ethyl)-1,3,4- oxadiazol-2-yl]-1,1-dioxo- 5-[[4-[5-(2,2,2- trifluoroethyl)-1,2,4- oxadiazol-3- yl]phenyl]methyl]-2,3- dihydro-126,5- benzothiazepin-4-one (*) | Intermediate 10 | 646.3 [M + H]⁺ |

(*) as hydrochloride salt

The examples of the following table were prepared in analogy to Example 32, using the appropriate benzyl bromide or carboxylic acid building block.

| | | | | MS, |
|---|---|---|---|---|
| | | | Building | ESI: |
| Ex. | Structure | Systematic Name | Block | m/z |
| 94 | 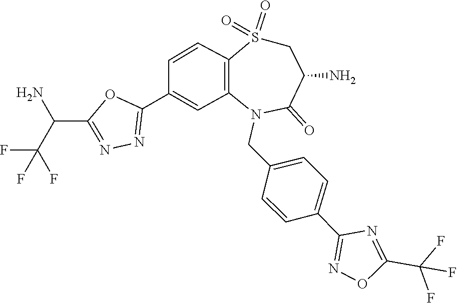 [SMILES: NC(c1nnc(-c2ccc3c(c2)N(Cc2ccc(-c4noc(C(F)(F)F)n4)cc2)C(=O)[C@@H](N)CS3(=O)=O)o1)C(F)(F)F] | (3R)-3-amino-7-[5-(1- amino-2,2,2-trifluoro- ethyl)-1,3,4-oxadiazol- 2-yl]-1,1-dioxo-5-[[4- [5-(trifluoromethyl)- 1,2,4-oxadiazol-3- yl]phenyl]methyl]-2,3- dihydro-1λ⁶,5- benzothiazepin-4-one (*) | 2-((tert- butoxycarbonyl) amino)- 3,3,3- trifluoropro- panoic acid (CAS 188030-43- 5) | 618.0 [M + H]⁺ |
| 95 | 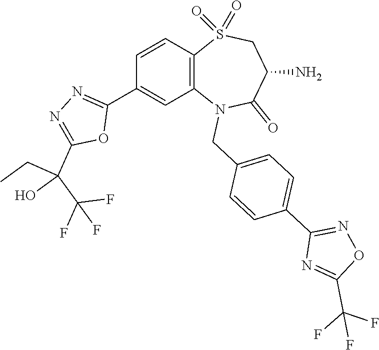 [SMILES: CCC(O)(c1nnc(-c2ccc3c(c2)N(Cc2ccc(-c4noc(C(F)(F)F)n4)cc2)C(=O)[C@@H](N)CS3(=O)=O)o1)C(F)(F)F] | (3R)-3-amino-7-[5-[1- hydroxy-1- (trifluoromethyl)propyl]- 1,3,4-oxadiazol-2-yl]- 1,1-dioxo-5-[[4-[5- (trifluoromethyl)-1,2,4- oxadiazol-3- yl]phenyl]methyl]-2,3- dihydro-1λ⁶,5- benzothiazepin-4-one (*) | 2-hydroxy- 2- trifluoromethyl- butyric acid (CAS 72114-82-0) | 647.3 [M + H]⁺ |
| 96 | 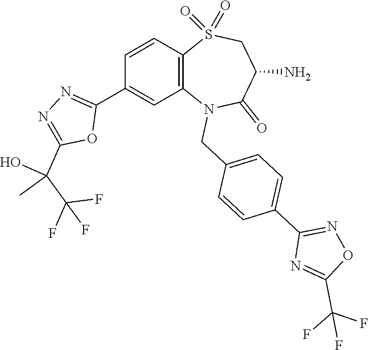 [SMILES: CC(O)(c1nnc(-c2ccc3c(c2)N(Cc2ccc(-c4noc(C(F)(F)F)n4)cc2)C(=O)[C@@H](N)CS3(=O)=O)o1)C(F)(F)F] | (3R)-3-amino-1,1- dioxo-7-[5-(2,2,2- trifluoro-1-hydroxy-1- methyl-ethyl)-1,3,4- oxadiazol-2-yl]-5-[[4- [5-(trifluoromethyl)- 1,2,4-oxadiazol-3- yl]phenyl]methyl]-2,3- dihydro-1λ⁶,5- benzothiazepin-4-one (*) | 3,3,3- trifluoro-2- hydroxy-2- methylpro- pionic acid (CAS 374- 35-6) | 633.3 [M + H]⁺ |
| 97 | 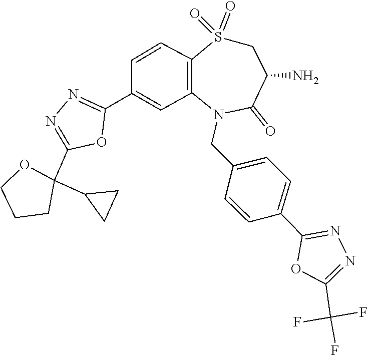 [SMILES: N[C@H]1CS(=O)(=O)c2ccc(-c3nnc(C4(C5CC5)CCCO4)o3)cc2N(Cc2ccc(-c3nnc(C(F)(F)F)o3)cc2)C1=O] | (3R)-3-amino-7-[5-(2- cyclopropyltetrahydro- furan-2-yl)-1,3,4- oxadiazol-2-yl]-1,1- dioxo-5-[[4-[5- (trifluoromethyl)-1,3,4- oxadiazol-2- yl]phenyl]methyl]-2,3- dihydro-1λ⁶,5- benzothiazepin-4-one (*) | 2- cyclopropyl tetrahydro- 2-furancarboxylic acid(CAS 1936371- 80-0, step c), 2-[4- (bromomethyl) phenyl]- 5-(trifluoro- methyl)-1,3,4- oxadiazole (CAS 2750234- 21-8, step e) | 631.3 [M + H]⁺ |

(*) as hydrochloride salt

### Example 34

### (3R)-3-amino-7-[5-(1-amino-2,2-dimethyl-propyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1)⁶,5-benzothiazepin-4-one

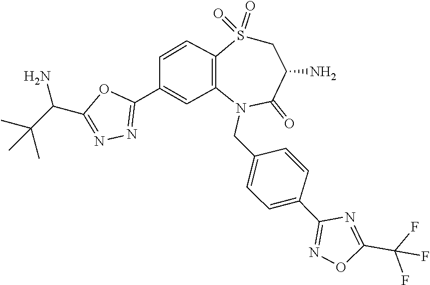 [SMILES: CC(C)(C)C(N)c1nnc(-c2ccc3c(c2)N(Cc2ccc(-c4noc(C(F)(F)F)n4)cc2)C(=O)[C@@H](N)CS3(=O)=O)o1]

### Step a) tert-butyl N-[(3R)-7-[[[2-(tert-butoxycarbonylamino)-3,3-dimethyl-butanoyl]amino]carbamoyl]-4-oxo-5-[[4-[5-(trifluoromethyl)-1,2,4-oxadiazol-3-yl]phenyl]methyl]-2,3-dihydro-1,5-benzothiazepin-3-yl]carbamate

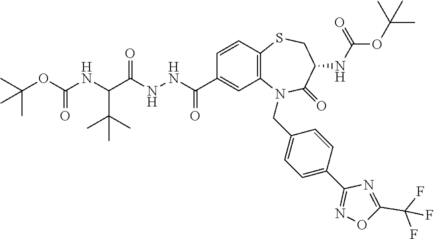 [SMILES: CC(C)(C)OC(=O)NC(C(=O)NNC(=O)c1ccc2c(c1)N(Cc1ccc(-c3noc(C(F)(F)F)n3)cc1)C(=O)[C@@H](NC(=O)OC(C)(C)C)CS2)C(C)(C)C]

The title compound was prepared from tert-butyl N-[(3R)-7-(hydrazinecarbonyl)-4-oxo-5-[[4-[5-(trifluoromethyl)-1,2,4-oxadiazol-3-yl]phenyl]methyl]-2,3-dihydro-1,5-benzothiazepin-3-yl]carbamate (100 mg, 0.168 mmol, 1.0 eq, Example 54, step c) and N-Boc-tert-leucine (255.05 mg, 1.1 mmol, 1.1 eq) in analogy to general procedure 4b and was obtained as light brown solid (650 mg, 0.82 mmol, 82% yield). MS (ESI): 692.3 [M+H-Boc]⁺

### Step b) tert-butyl N-[(3R)-7-[5-[1]-(tert-butoxycarbonylamino)-2,2-dimethyl-propyl]-1,3,4-oxadiazol-2-yl]-4-oxo-5-[[4-[5-(trifluoromethyl)-1,2,4-oxadiazol-3-yl]phenyl]methyl]-2,3-dihydro-1,5-benzothiazepin-3-yl]carbamate

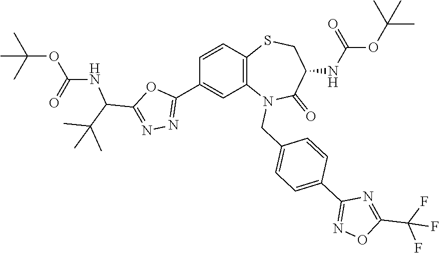 [SMILES: CC(C)(C)OC(=O)NC(c1nnc(-c2ccc3c(c2)N(Cc2ccc(-c4noc(C(F)(F)F)n4)cc2)C(=O)[C@@H](NC(=O)OC(C)(C)C)CS3)o1)C(C)(C)C]

The title compound was prepared from tert-butyl N-[(3R)-7-[[[2-(tert-butoxycarbonylamino)-3,3-dimethyl-butanoyl]amino]carbamoyl]-4-oxo-5-[[4-[5-(trifluoromethyl)-1,2,4-oxadiazol-3-yl]phenyl]methyl]-2,3-dihydro-1,5-benzothiazepin-3-yl]carbamate (630 mg, 0.8 mmol) in analogy to general procedure 5a and was obtained as light yellow solid (800 mg, 1.03 mmol, 123% yield). MS (ESI): 774.4 [M+H]⁺.

### Step c) tert-butyl N-[(3R)-7-[5-[1]-(tert-butoxycarbonylamino)-2,2-dimethyl-propyl]-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate

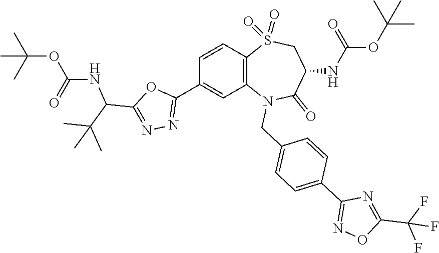 [SMILES: CC(C)(C)OC(=O)NC(c1nnc(-c2ccc3c(c2)N(Cc2ccc(-c4noc(C(F)(F)F)n4)cc2)C(=O)[C@@H](NC(=O)OC(C)(C)C)CS3(=O)=O)o1)C(C)(C)C]

The title compound was prepared from tert-butyl N-[(3R)-7-[5-[1-(tert-butoxycarbonylamino)-2,2-dimethyl-propyl]-1,3,4-oxadiazol-2-yl]-4-oxo-5-[[4-[5-(trifluoromethyl)-1,2,4-oxadiazol-3-yl]phenyl]methyl]-2,3-dihydro-1,5-benzothiazepin-3-yl]carbamate (750 mg, 0.97 mmol) in analogy to general procedure 10 and was obtained as yellow solid (500 mg, 0.62 mmol, 64% yield). MS (ESI): 828.3 [M+H]⁺.

### Step d) (3R)-3-amino-7-[5-(1-amino-2,2-dimethyl-propyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one

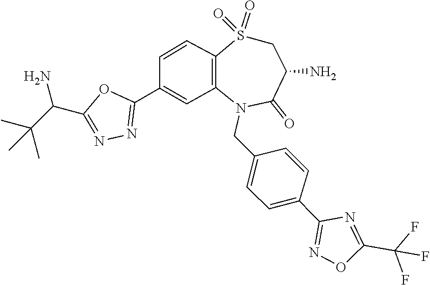 [SMILES: CC(C)(C)C(N)c1nnc(-c2ccc3c(c2)N(Cc2ccc(-c4noc(C(F)(F)F)n4)cc2)C(=O)[C@@H](N)CS3(=O)=O)o1]

The title compound was prepared form tert-butyl N-[(3R)-7-[5-[1-(tert-butoxycarbonylamino)-2,2-dimethyl-propyl]-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (150 mg, 0.19 mmol) in analogy to general procedure 11c and was obtained as light yellow solid (111.7 mg, 0.16 mmol, 86% yield). MS (ESI): 606.0 [M+H]⁺.

The examples of the following table were prepared in analogy to Example 34, using the appropriate carboxylic acid building block.

| | | | | MS, ESI: |
|---|---|---|---|---|
| Ex. | Structure | Systematic Name | Building Block | m/z |
| 35 | 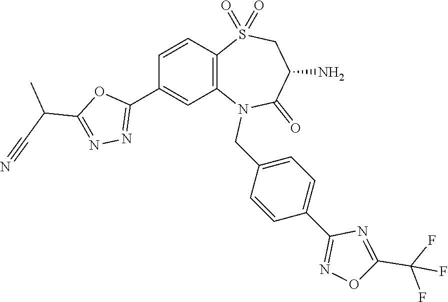 [SMILES: CC(C#N)c1nnc(-c2ccc3c(c2)N(Cc2ccc(-c4noc(C(F)(F)F)n4)cc2)C(=O)[C@@H](N)CS3(=O)=O)o1] | 2-[5-[(3R)-3-amino-1,1,4- trioxo-5-[[4-[5-(trifluoromethyl)- 1,2,4-oxadiazol-3- yl]phenyl]methyl]- 2,3-dihydro-1λ⁶,5- benzothiazepin-7-yl]-1,3,4- oxadiazol-2-yl]propanenitrile | 2-cyanopropanoic acid (CAS 632- 07-5) | 573.9 [M + H]⁺ |
| 36 | 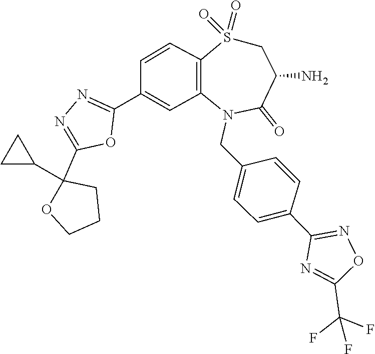 [SMILES: N[C@H]1CS(=O)(=O)c2ccc(-c3nnc(C4(C5CC5)CCCO4)o3)cc2N(Cc2ccc(-c3noc(C(F)(F)F)n3)cc2)C1=O] | (3R)-3-amino-7-[5-(2- cyclopropyltetrahydrofuran- 2-yl)-1,3,4-oxadiazol-2- yl]-1,1-dioxo-5-[[4-[5- (trifluoromethyl)-1,2,4- oxadiazol-3-yl]phenyl] methyl]-2,3-dihydro-1λ⁶,5- benzothiazepin-4-one (*) | 2- cyclopropyloxolane- 2-carboxylic acid (CAS 1936371- 80-0) | 631.3 [M + H]⁺ |
| 37 | 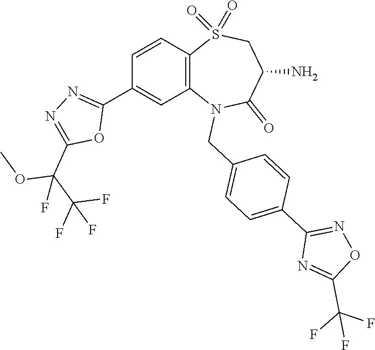 [SMILES: COC(F)(c1nnc(-c2ccc3c(c2)N(Cc2ccc(-c4noc(C(F)(F)F)n4)cc2)C(=O)[C@@H](N)CS3(=O)=O)o1)C(F)(F)F] | (3R)-3-amino-1,1-dioxo- 7-[5-(1,2,2,2-tetrafluoro-1- methoxy-ethyl)-1,3,4- oxadiazol-2-yl]-5-[[4-[5- (trifluoromethyl)-1,2,4- oxadiazol-3-yl]phenyl] methyl]-2,3-dihydro-1λ⁶,5- benzothiazepin-4-one (*) | 2,3,3,3- tetrafluoro-2- methoxypropanoic acid (CAS 10186-64-8) | 651.3 [M + H]⁺ |

(*) as hydrochloride salt

### Example 38

### 2-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]-2-methyl-propanenitrile

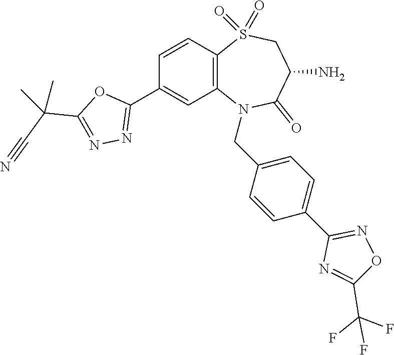 [SMILES: CC(C)(C#N)c1nnc(-c2ccc3c(c2)N(Cc2ccc(-c4noc(C(F)(F)F)n4)cc2)C(=O)[C@@H](N)CS3(=O)=O)o1]

### Step a) tert-butyl N-[(3R)-7-[[(2-cyano-2-methyl-propanoyl)amino]carbamoyl]-4-oxo-3,5-dihydro-2H-1,5-benzothiazepin-3-yl]carbamate

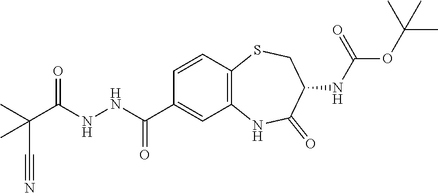 [SMILES: CC(C)(C)OC(=O)N[C@H]1CSc2ccc(C(=O)NNC(=O)C(C)(C)C#N)cc2NC1=O]

The title compound was prepared tert-butyl N-[(3R)-7-(hydrazinecarbonyl)-4-oxo-3,5-dihydro-2H-1,5-benzothiazepin-3-yl]carbamate (100 mg, 0.270 mmol, 1.0 eq, Example 19, step b) and 2-cyano-2-methylpropionic acid (36.59 mg, 0.323 mmol, 1.2 eq, CAS 22426-30-8) in analogy to general procedure 4a and was obtained as white solid (114.5 mg, 95%). MS (ESI): 446.2 [M−H]

### Step b) tert-butyl N-[(3R)-7-[5-(1-cyano-1-methyl-ethyl)-1,3,4-oxadiazol-2-yl]-4-oxo-3,5-dihydro-2H-1,5-benzothiazepin-3-yl]carbamate

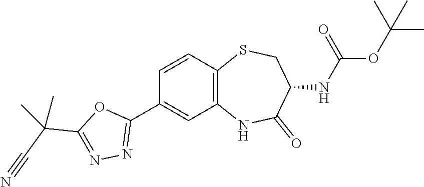 [SMILES: CC(C)(C)OC(=O)N[C@H]1CSc2ccc(-c3nnc(C(C)(C)C#N)o3)cc2NC1=O]

The title compound was prepared from tert-butyl N-[(3R)-7-[[(2-cyano-2-methyl-propanoyl)amino]carbamoyl]-4-oxo-3,5-dihydro-2H-1,5-benzothiazepin-3-yl]carbamate (114.5 mg, 0.256 mmol) in analogy to general procedure 5b and was obtained as white solid (83.3 mg, 76%). MS (ESI): 374.1 [M-isobutene+H]⁺.

### Step c) tert-butyl N-[(3R)-7-[5-(1-cyano-1-methyl-ethyl)-1,3,4-oxadiazol-2-yl]1,1,4-trioxo-3,5-dihydro-2H-1λ⁶,5-benzothiazepin-3-yl]carbamate

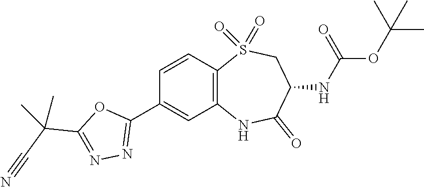 [SMILES: CC(C)(C)OC(=O)N[C@H]1CS(=O)(=O)c2ccc(-c3nnc(C(C)(C)C#N)o3)cc2NC1=O]

The title compound was prepared from tert-butyl N-[(3R)-7-[5-(1-cyano-1-methyl-ethyl)-1,3,4-oxadiazol-2-yl]-4-oxo-3,5-dihydro-2H-1,5-benzothiazepin-3-yl]carbamate (83.3 mg, 0.194 mmol) in analogy to general procedure 10 and was obtained as white solid (72.7 mg, 81%). MS (ESI): 406.1 [M-isobutene+H]⁺

### Step d) tert-butyl N-[(3R)-7-[5-(1-cyano-1-methyl-ethyl)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶, 5-benzothiazepin-3-yl]carbamate

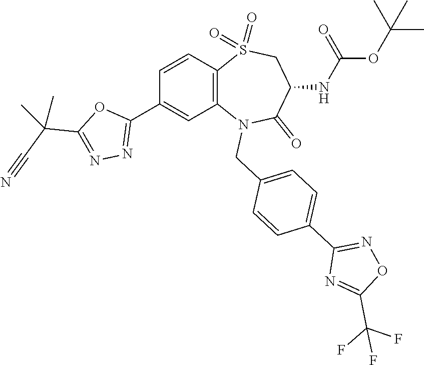 [SMILES: CC(C)(C)OC(=O)N[C@H]1CS(=O)(=O)c2ccc(-c3nnc(C(C)(C)C#N)o3)cc2N(Cc2ccc(-c3noc(C(F)(F)F)n3)cc2)C1=O]

The title compound was prepared from tert-butyl N-[(3R)-7-[5-(1-cyano-1-methyl-ethyl)-1,3,4-oxadiazol-2-yl]-1,1,4-trioxo-3,5-dihydro-2H-1λ⁶,5-benzothiazepin-3-yl]carbamate (36 mg, 0.078 mmol, 1.0) and 3-[4-(bromomethyl)phenyl]-5-(trifluoromethyl)-1,2,4-oxadiazole (35.9 mg, 0.117 mmol, 1.5 eq, CAS 2093101-98-3) in analogy ot general procedure 1a and was obtained as white solid (33 mg, 62%). MS (ESI): 632.1 [M-isobutene+H]⁺

### Step e) 2-[5-[(3R)-3-amino-1,1,4-trioxo-5-[[4-[5-(trifluoromethyl)-1,2,4-oxadiazol-3-yl]phenyl]methyl]-2,3-dihydro-1λ⁶, 5-benzothiazepin-7-yl]-1,3,4-oxadiazol-2-yl]-2-methyl-propanenitrile

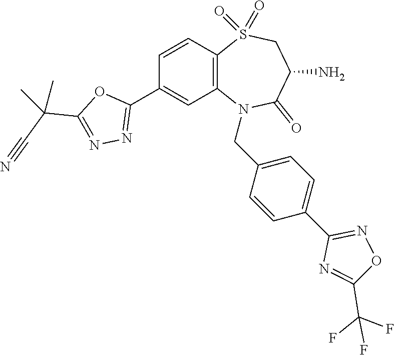 [SMILES: CC(C)(C#N)c1nnc(-c2ccc3c(c2)N(Cc2ccc(-c4noc(C(F)(F)F)n4)cc2)C(=O)[C@@H](N)CS3(=O)=O)o1]

The title compound was prepared from tert-butyl N-[(3R)-7-[5-(1-cyano-1-methyl-ethyl)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (33 mg, 0.048 mmol) in analogy to general procedure 11a and was obtained as off-white solid, as hydrochloride salt (29.5 mg, 99%). MS (ESI): 632.4 [M+H]⁺

The examples of the following table were prepared in analogy to Example 38, using the appropriate carboxylic acid and/or benzyl bromide building block in the indicated synthesis step.

| | | | Building | MS, |
|---|---|---|---|---|
| Ex. | Structure | Systematic Name | Block | ESI: m/z |
| 39 | 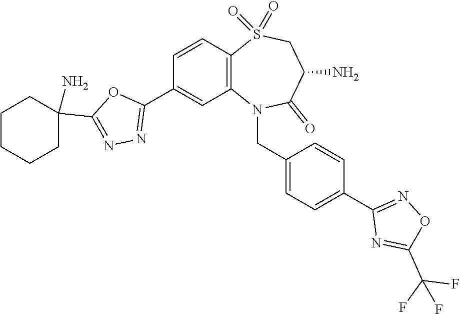 [SMILES: N[C@H]1CS(=O)(=O)c2ccc(-c3nnc(C4(N)CCCCC4)o3)cc2N(Cc2ccc(-c3noc(C(F)(F)F)n3)cc2)C1=O] | (3R)-3-amino-7-[5- (1- aminocyclohexyl)- 1,3,4-oxadiazol-2- yl]-1,1-dioxo-5-[[4- [5- (trifluoromethyl)- 1,2,4-oxadiazol-3- yl]phenyl]methyl]- 2,3-dihydro-1λ⁶,5- benzothiazepin-4- one (*) | 1-(tert- butoxycarbon ylamino)-1- cyclohexane- carboxylic acid (CAS 115951-16-1, step a) | 618.3 [M + H]⁺ |
| 40 | 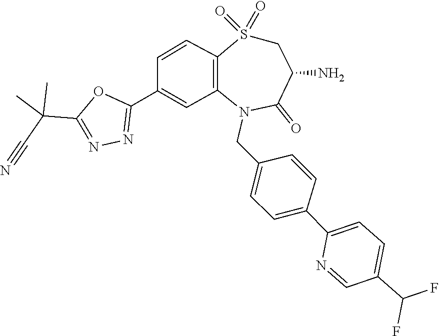 [SMILES: CC(C)(C#N)c1nnc(-c2ccc3c(c2)N(Cc2ccc(-c4ccc(C(F)F)cn4)cc2)C(=O)[C@@H](N)CS3(=O)=O)o1] | 2-[5-[(3R)-3- amino-5-[[4-[5- (difluoromethyl)-2- pyridyl]phenyl] methyl]-1,1,4-trioxo- 2,3-dihydro-1λ⁶,5- benzothiazepin-7- yl]-1,3,4- oxadiazol-2-yl]-2- methyl- propanenitrile (*) | Intermediate 11 (step d) | 579.3 [M + H]⁺ |

(*) as hydrochloride salt

### Example 41

### (3R)-3-amino-7-[5-(1-aminocyclohexyl)-1,3,4-oxadiazol-2-yl]-1,1-dioxo-5-[[14-[5-(trifluoromethyl)-2-pyridyl]phenyl]methyl]-2,3-dihydro-1λ⁶,5-benzothiazepin-4-one

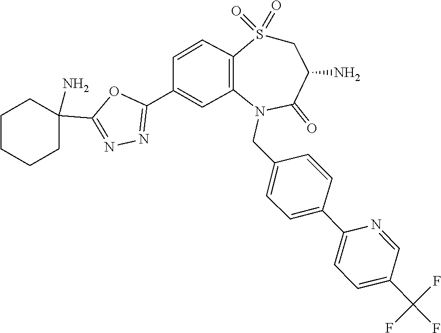 [SMILES: N[C@H]1CS(=O)(=O)c2ccc(-c3nnc(C4(N)CCCCC4)o3)cc2N(Cc2ccc(-c3ccc(C(F)(F)F)cn3)cc2)C1=O]

### Step a) tert-butyl N-[(3R)-7-[[[1-(tert-butoxycarbonylamino)cyclohexanecarbonyl]amino]carbamoyl]-4-oxo-3,5-dihydro-2H-1,5-benzothiazepin-3-yl]carbamate

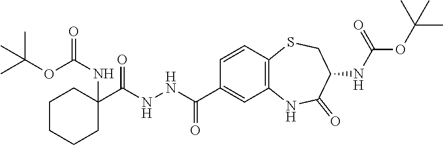 [SMILES: CC(C)(C)OC(=O)N[C@H]1CSc2ccc(C(=O)NNC(=O)C3(NC(=O)OC(C)(C)C)CCCCC3)cc2NC1=O]

The title compound was prepared from tert-butyl N-[(3R)-7-(hydrazinecarbonyl)-4-oxo-3,5-dihydro-2H-1,5-benzothiazepin-3-yl]carbamate (150 mg, 0.425 mmol, 1.0 eq, Example 19, step b) and 1-(tert-butoxycarbonylamino)-1-cyclohexanecarboxylic acid (124.27 mg, 0.51 mmol, 1.2 eq, CAS 115951-16-1) in analogy to general procedure 4a and was obtained as light yellow solid (207.5 mg, 76%). MS (ESI): 576.3 [M−H]

### Step b) tert-butyl N-[(3R)-7-[5-[1-(tert-butoxycarbonylamino)cyclohexyl]-1,3,4-oxadiazol-2-yl]-4-oxo-3,5-dihydro-2H-1,5-benzothiazepin-3-yl]carbamate

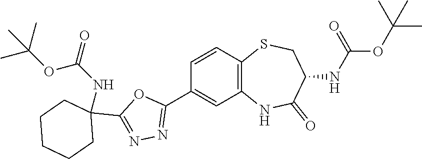 [SMILES: CC(C)(C)OC(=O)N[C@H]1CSc2ccc(-c3nnc(C4(NC(=O)OC(C)(C)C)CCCCC4)o3)cc2NC1=O]

The title compound was prepared from tert-butyl N-[(3R)-7-[[[1-(tert-butoxycarbonylamino)cyclohexanecarbonyl]amino]carbamoyl]-4-oxo-3,5-dihydro-2H-1,5-benzothiazepin-3-yl]carbamate (67 mg, 0.095 mmol) in analogy to general procedure 5b and was obtained as white solid (48 mg, 80%). MS (ESI): 560.3 [M+H]⁺

### Step c) tert-butyl N-[(3R)-7-[5-[1-(tert-butoxycarbonylamino)cyclohexyl]-1,3,4-oxadiazol-2-yl]-1,1,4-trioxo-3,5-dihydro-2H-1λ⁶,5-benzothiazepin-3-yl]carbamate

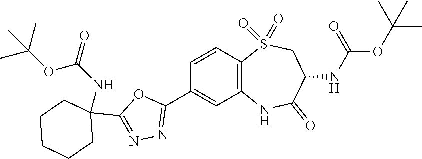 [SMILES: CC(C)(C)OC(=O)N[C@H]1CS(=O)(=O)c2ccc(-c3nnc(C4(NC(=O)OC(C)(C)C)CCCCC4)o3)cc2NC1=O]

The title compound was prepared from tert-butyl N-[(3R)-7-[5-[1-(tert-butoxycarbonylamino)cyclohexyl]-1,3,4-oxadiazol-2-yl]-4-oxo-3,5-dihydro-2H-1,5-benzothiazepin-3-yl]carbamate (48 mg, 0.076 mmol) in analogy to general procedure 10 and was obtained as white solid (46 mg, 96%). MS (ESI): 480.1 [M-2xisobutene+H]⁺

### Step d) tert-butyl N-[(3R)-7-[5-[1-(tert-butoxycarbonylamino)cyclohexyl]-1,3,4-oxadiazol-2-yl]-1,1,4-trioxo-5-[[4-[5-(trifluoromethyl)-2-pyridyl]phenyl]methyl]-2,3-dihydro-1λ⁶, 5-benzothiazepin-3-yl]carbamate

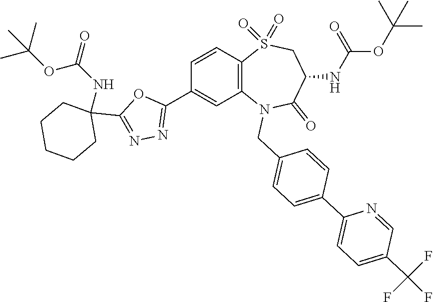 [SMILES: CC(C)(C)OC(=O)N[C@H]1CS(=O)(=O)c2ccc(-c3nnc(C4(NC(=O)OC(C)(C)C)CCCCC4)o3)cc2N(Cc2ccc(-c3ccc(C(F)(F)F)cn3)cc2)C1=O]

The title compound was prepared from tert-butyl N-[(3R)-7-[5-[1-(tert-butoxycarbonylamino)cyclohexyl]-1,3,4-oxadiazol-2-yl]-1,1,4-trioxo-5-[[4-[5-(trifluoromethyl)-2-pyridyl]phenyl]methyl]-2,3-dihydro-1λ⁶,5-benzothiazepin-3-yl]carbamate (46 mg, 0.073 mmol, 1.0 eq) and 2-[4-(bromomethyl)phenyl]-5-(trifluoromethyl)pyridine (23.1 mg, 0.073 μmol, 1.0 eq, CAS 1056641-21-4) in analogy to general procedure 1a and was obtained as white solid (49 mg, 81%). MS (ESI): 827.7 [M+H]⁺

### Step e) (3R)-3-amino-7-[5-(1-aminocyclohexyl)-1,3,4-oxadiazol-2-yl]-1,1-dioxo-5-[[4-[5-(trifluoromethyl)-2-pyridyl]phenyl]methyl]-2,3-dihydro-1λ⁶,5-benzothiazepin-4-one

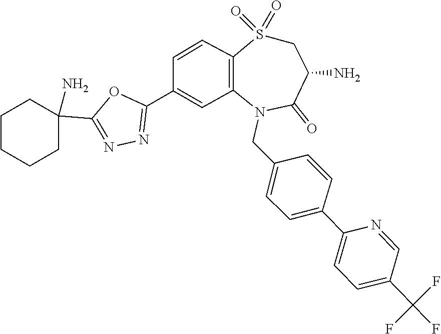 [SMILES: N[C@H]1CS(=O)(=O)c2ccc(-c3nnc(C4(N)CCCCC4)o3)cc2N(Cc2ccc(-c3ccc(C(F)(F)F)cn3)cc2)C1=O]

The title compound was prepared from tert-butyl N-[(3R)-7-[5-[1-(tert-butoxycarbonylamino)cyclohexyl]-1,3,4-oxadiazol-2-yl]-1,1,4-trioxo-5-[[4-[5-(trifluoromethyl)-2-pyridyl]phenyl]methyl]-2,3-dihydro-1λ⁶,5-benzothiazepin-3-yl]carbamate (49 mg, 0.059 mmol) in analogy to general procedure 11a and was obtained as off-white solid, as hydrochloride salt (42 mg, 101%). MS (ESI): 627.3 [M+H]⁺

The examples of the following table were prepared in analogy to Example 41, using the appropriate benzyl bromide building block.

| | | | | MS, |
|---|---|---|---|---|
| | | Systematic | Building | ESI: |
| Ex. | Structure | Name | Block | m/z |
| 42 | 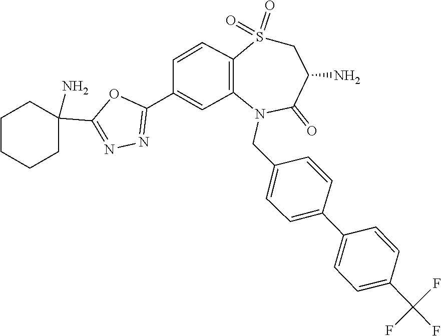 [SMILES: N[C@H]1CS(=O)(=O)c2ccc(-c3nnc(C4(N)CCCCC4)o3)cc2N(Cc2ccc(-c3ccc(C(F)(F)F)cc3)cc2)C1=O] | (3R)-3-amino-7-[5-(1- aminocyclohexyl)- 1,3,4-oxadiazol-2-yl]- 1,1-dioxo-5-[[4-[4- (trifluoromethyl)phenyl] phenyl]methyl]-2,3- dihydro-1λ⁶,5- benzothiazepin-4-one (*) | 4- (bromomethyl)- 4′-(trifluoro- methyl)- 1,1′- biphenyl (CAS 613241- 14-8) | 626.3 [M + H]⁺ |
| 43 | 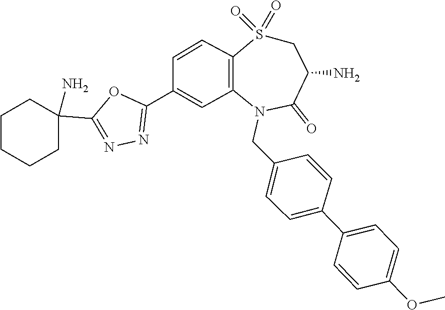 [SMILES: COc1ccc(-c2ccc(CN3C(=O)[C@@H](N)CS(=O)(=O)c4ccc(-c5nnc(C6(N)CCCCC6)o5)cc43)cc2)cc1] | (3R)-3-amino-7-[5-(1- aminocyclohexyl)- 1,3,4-oxadiazol-2-yl]- 5-[4-(4- methoxyphenyl)phenyl] methyl]-1,1-dioxo-2,3- dihydro-1λ⁶,5- benzothiazepin-4-one (*) | 4- (bromomethyl)- 4′-methoxy- 1,1′- biphenyl (CAS 20854-61- 9) | 588.3 [M + H]⁺ |
| 44 | 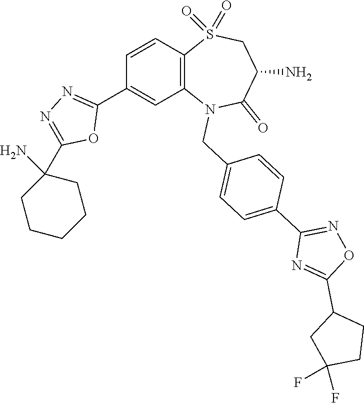 [SMILES: N[C@H]1CS(=O)(=O)c2ccc(-c3nnc(C4(N)CCCCC4)o3)cc2N(Cc2ccc(-c3noc(C4CCC(F)(F)C4)n3)cc2)C1=O] | (3R)-3-amino-7-[5-(1- aminocyclohexyl)- 1,3,4-oxadiazol-2-yl]- 5-[[4-[5-(3,3- difluorocyclopentyl)- 1,2,4-oxadiazol-3- yl]phenyl]methyl]-1,1- dioxo-2,3-dihydro- 1λ⁶,5-benzothiazepin-4- one (*) | Intermediate 12 | 654.3 [M + H]⁺ |
| 98 | 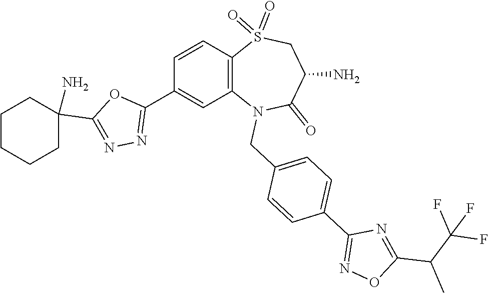 [SMILES: CC(c1nc(-c2ccc(CN3C(=O)[C@@H](N)CS(=O)(=O)c4ccc(-c5nnc(C6(N)CCCCC6)o5)cc43)cc2)no1)C(F)(F)F] | (3R)-3-amino-7-[5-(1- aminocyclohexyl)- 1,3,4-oxadiazol-2-yl]- 1,1-dioxo-5-[[4-[5- (2,2,2-trifluoro-1- methyl-ethyl)-1,2,4- oxadiazol-3- yl]phenyl]methyl]-2,3- dihydro-1λ⁶,5- benzothiazepin-4-one (*) | Intermediate 49 | 646.3 [M + H]⁺ |

(*) as hydrochloride salt

### Example 45

### 2-[5-[(3R)-3-amino-5-[[4-(4-methoxyphenyl)phenyl]methyl]-1,1,4-trioxo-2,3-dihydro-1λ⁶,5-benzothiazepin-7-yl]-1,3,4-oxadiazol-2-yl]-2-methyl-propanenitrile

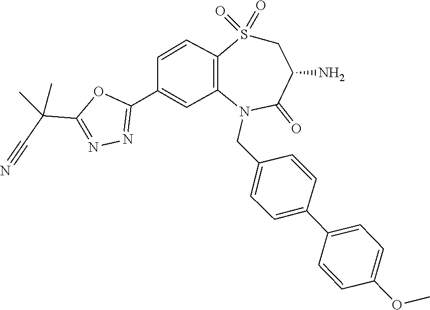 [SMILES: COc1ccc(-c2ccc(CN3C(=O)[C@@H](N)CS(=O)(=O)c4ccc(-c5nnc(C(C)(C)C#N)o5)cc43)cc2)cc1]

### Step a) tert-butyl N-[(3R)-7-[5-(1-cyano-1-methyl-ethyl)-1,3,4-oxadiazol-2-yl]-5-[[4-(4-methoxyphenyl)phenyl]methyl]-1,1,4-trioxo-2,3-dihydro-1λ⁶, 5-benzothiazepin-3-yl]carbamate

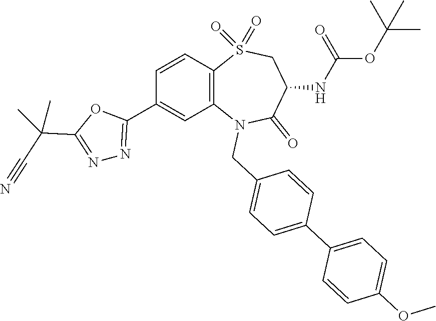 [SMILES: COc1ccc(-c2ccc(CN3C(=O)[C@@H](NC(=O)OC(C)(C)C)CS(=O)(=O)c4ccc(-c5nnc(C(C)(C)C#N)o5)cc43)cc2)cc1]

To a solution tert-butyl N-[(3R)-7-[5-(1-cyano-1-methyl-ethyl)-1,3,4-oxadiazol-2-yl]-1,1,4-trioxo-3,5-dihydro-2H-1λ⁶,5-benzothiazepin-3-yl]carbamate (36 mg, 0.078 mmol, 1.0 eq, Example 38, step c) and 4-(chloromethyl)-4′-methoxy-1,1′-biphenyl (27.2 mg, 0.12 mmol, 1.5 eq, CAS 93258-73-2) in analogy to general procedure 1a and was obtained as white solid (35.7 mg, 70%). MS (ESI): 602.2 [M-isobutene+H]⁺

### Step b) 2-[5-[(3R)-3-amino-5-[[4-(4-methoxyphenyl)phenyl]methyl]-1,1,4-trioxo-2,3-dihydro-1λ⁶,5-benzothiazepin-7-yl]-1,3,4-oxadiazol-2-yl]-2-methyl-propanenitrile

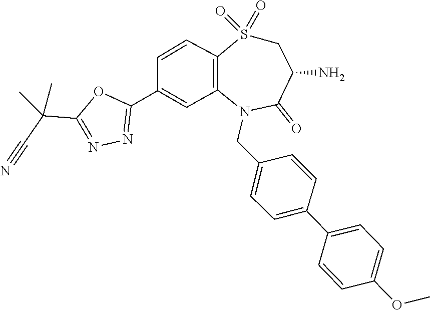 [SMILES: COc1ccc(-c2ccc(CN3C(=O)[C@@H](N)CS(=O)(=O)c4ccc(-c5nnc(C(C)(C)C#N)o5)cc43)cc2)cc1]

The title compound was prepared from tert-butyl N-[(3R)-7-[5-(1-cyano-1-methyl-ethyl)-1,3,4-oxadiazol-2-yl]-5-[[4-(4-methoxyphenyl)phenyl]methyl]-1,1,4-trioxo-2,3-dihydro-1λ⁶, 5 benzothiazepin-3-yl]carbamate (35 mg, 0.053 mmol) in analogy to general procedure a and was obtained as off-white solid, as hydrochloride salt (31 mg, 980%). MS (ESI): 558.3 [M-isobutene+H]⁺

The examples of the following table were prepared in analogy to Example 45, using the appropriate benzyl bromide building block.

| | | | | MS, |
|---|---|---|---|---|
| | | | Building | ESI: |
| Ex. | Structure | Systematic Name | Block | m/z |
| 46 | 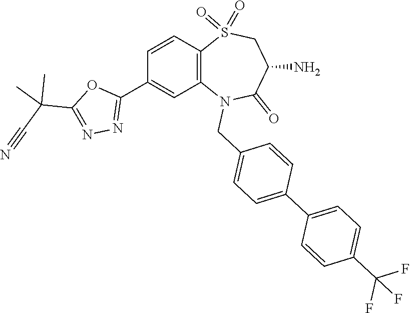 [SMILES: CC(C)(C#N)c1nnc(-c2ccc3c(c2)N(Cc2ccc(-c4ccc(C(F)(F)F)cc4)cc2)C(=O)[C@@H](N)CS3(=O)=O)o1] | 2-[5-[(3R)-3-amino- 1,1,4-trioxo-5-[[4-[5- (trifluoromethyl)-2- pyridyl]phenyl]methyl]- 2,3-dihydro-1λ⁶,5- benzothiazepin-7-yl]- 1,3,4-oxadiazol-2-yl]- 2-methyl- propanenitrile (*) | 4- (bromo- methyl)-4′- (trifluoro- methyl)-1,1′- biphenyl (CAS 613241- 14-8) | 596.2 [M + H]⁺ |
| 47 | 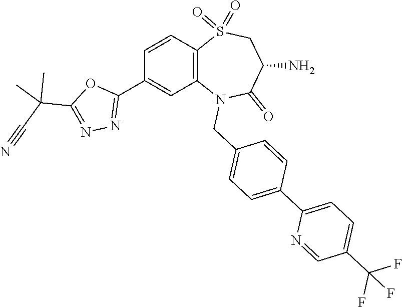 [SMILES: CC(C)(C#N)c1nnc(-c2ccc3c(c2)N(Cc2ccc(-c4ccc(C(F)(F)F)cn4)cc2)C(=O)[C@@H](N)CS3(=O)=O)o1] | 2-[5-[(3R)-3-amino- 1,1,4-trioxo-5-[[4-[4- (trifluoromethyl)phenyl] phenyl]methyl]- 2,3-dihydro- 1λ⁶,5-benzothiazepin- 7-yl]-1,3,4-oxadiazol-2- yl]-2-methyl propane- nitrile (*) | 2-[4- (bromo- methyl) phenyl]- 5- (trifluoro- methyl) pyridine (CAS 1056641- 21-4) | 597.2 [M + H]⁺] |
| 100 | 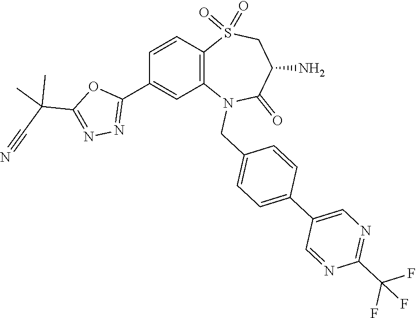 [SMILES: CC(C)(C#N)c1nnc(-c2ccc3c(c2)N(Cc2ccc(-c4cnc(C(F)(F)F)nc4)cc2)C(=O)[C@@H](N)CS3(=O)=O)o1] | 2-[5-[(3R)-3-amino- 1,1,4-trioxo-5-[[4-[2- (trifluoromethyl) pyrimidin-5-yl] phenyl]methyl]-2,3- dihydro-1λ⁶,5- benzothiazepin-7-yl]- 1,3,4-oxadiazol-2-yl]- 2-methyl- propanenitrile (*) | Inter- mediate 19 | 598.3 [M + H]⁺ |
| 101 | 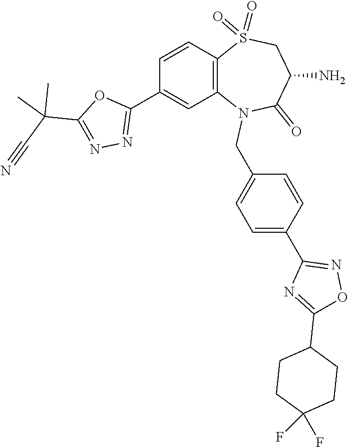 [SMILES: CC(C)(C#N)c1nnc(-c2ccc3c(c2)N(Cc2ccc(-c4noc(C5CCC(F)(F)CC5)n4)cc2)C(=O)[C@@H](N)CS3(=O)=O)o1] | 2-[5-[(3R)-3-amino- 5-[[4-[5-(4,4- difluorocyclohexyl)- 1,2,4-oxadiazol-3- yl]phenyl]methyl]- 1,1,4-trioxo-2,3- dihydro-1λ⁶,5- benzothiazepin-7-yl]- 1,3,4-oxadiazol-2-yl]- 2-methyl- propanenitrile (*) | Inter- mediate 20 | 638.2 [M + H]⁺ |
| 102 | 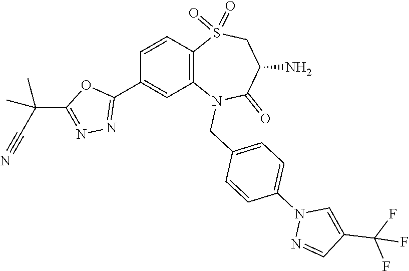 [SMILES: CC(C)(C#N)c1nnc(-c2ccc3c(c2)N(Cc2ccc(-n4cc(C(F)(F)F)cn4)cc2)C(=O)[C@@H](N)CS3(=O)=O)o1] | 2-[5-[(3R)-3-amino- 1,1,4-trioxo-5-[[4-[4- (trifluoromethyl) pyrazol-1-yl]phenyl] methyl]-2,3-dihydro- 1λ⁶,5-benzothiazepin- 7-yl]- 1,3,4-oxadiazol-2-yl]- 2-methyl- propanenitrile | 1-[4- (chloro- methyl) phenyl]-4- (trifluoro- methyl)- 1H- pyrazole (CAS 1486714- 19-5) | 586.4 [M + H]⁺ |
| 104 | 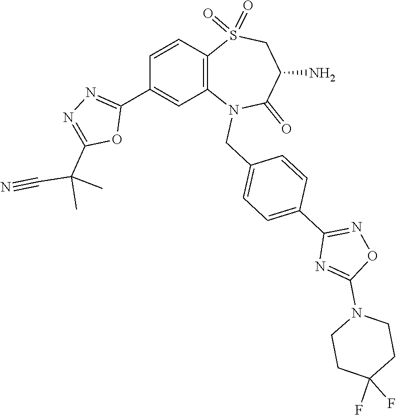 [SMILES: CC(C)(C#N)c1nnc(-c2ccc3c(c2)N(Cc2ccc(-c4noc(N5CCC(F)(F)CC5)n4)cc2)C(=O)[C@@H](N)CS3(=O)=O)o1] | 2-[5-[(3R)-3-amino- 5-[[4-[5-(4,4- difluoro-1-piperidyl)- 1,2,4-oxadiazol-3- yl]phenyl]methyl]- 1,1,4-trioxa-2,3- dihydro-1λ⁶,5- benzothiazepin-7-yl]- 1,3,4-oxadiazol-2-yl]- 2-methyl- propanenitrile (*) | Inter- mediate 22 | 639.3 [M + H]⁺ |
| 105 | 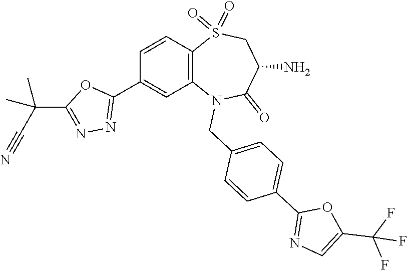 [SMILES: CC(C)(C#N)c1nnc(-c2ccc3c(c2)N(Cc2ccc(-c4ncc(C(F)(F)F)o4)cc2)C(=O)[C@@H](N)CS3(=O)=O)o1] | 2-[5-[(3R)-3-amino- 1,1,4-trioxo-5-[[4-[5- (trifluoromethyl)oxazol- 2-yl]phenyl]methyl]- 2,3-dihydro-1λ⁶,5- benzothiazepin-7-yl]- 1,3,4-oxadiazol-2-yl]- 2-methyl- propanenitrile (*) | Inter- mediate 23 | 587.3 [M + H]⁺ |
| 106 | 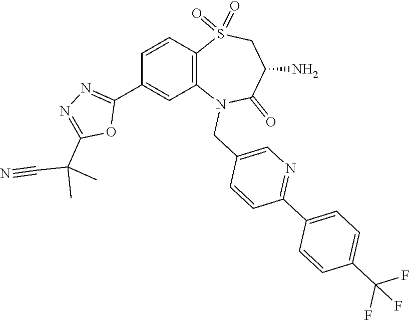 [SMILES: CC(C)(C#N)c1nnc(-c2ccc3c(c2)N(Cc2ccc(-c4ccc(C(F)(F)F)cc4)nc2)C(=O)[C@@H](N)CS3(=O)=O)o1] | 2-[5-[(3R)-3-amino- 1,1,4-trioxo-5-[[6-[4- (trifluoromethyl) phenyl]-3- pyridyl]methyl]-2,3- dihydro-1λ⁶,5- benzothiazepin-7-yl]- 1,3,4-oxadiazol-2-yl]- 2-methyl- propanenitrile (*) | 5-(Chloro- methyl)- 2- [4- (trifluoro- methyl) phenyl] pyridine (CAS 851507- 54-5) | 597.2 [M + H]⁺ |
| 107 | 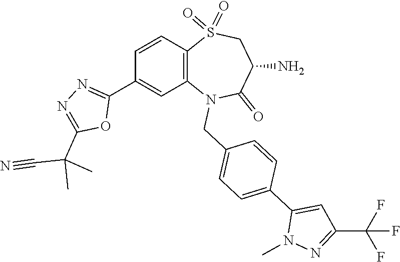 [SMILES: Cn1nc(C(F)(F)F)cc1-c1ccc(CN2C(=O)[C@@H](N)CS(=O)(=O)c3ccc(-c4nnc(C(C)(C)C#N)o4)cc32)cc1] | 2-[5-[(3R)-3-amino- 5-[[4-[2-methyl-5- (trifluoromethyl) pyrazol-3- yl]phenyl]methyl]- 1,1,4-trioxo-2,3- dihydro-1λ⁶,5- benzothiazepin-7-yl]- 1,3,4-oxadiazol-2-yl]- 2-methyl- propanenitrile (*) | Inter- mediate 24 | 600.2 [M + H]⁺ |
| 108 | 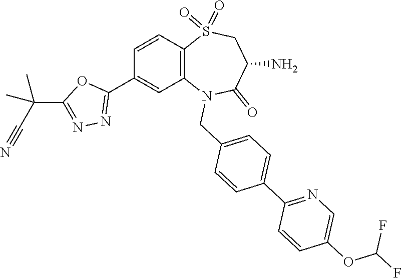 [SMILES: CC(C)(C#N)c1nnc(-c2ccc3c(c2)N(Cc2ccc(-c4ccc(OC(F)F)cn4)cc2)C(=O)[C@@H](N)CS3(=O)=O)o1] | 2-[5-[(3R)-3-amino- 5-[[4-[5- (difluoromethoxy)-2- pyridyl]phenyl] methyl]- 1,1,4-trioxo-2,3- dihydro-1λ⁶,5- benzothiazepin-7-yl]- 1,3,4-oxadiazol-2-yl]- 2-methyl- propanenitrile (*) | Inter- mediate 25 | 595.1 [M + H]⁺ |
| 109 | 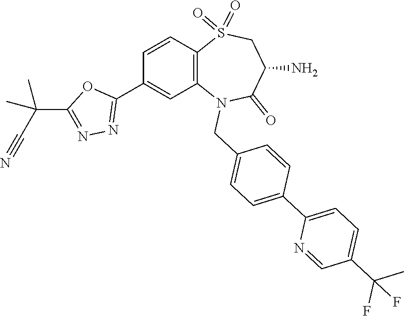 [SMILES: CC(F)(F)c1ccc(-c2ccc(CN3C(=O)[C@@H](N)CS(=O)(=O)c4ccc(-c5nnc(C(C)(C)C#N)o5)cc43)cc2)nc1] | 2-[5-[(3R)-3-amino- 5-[[4-[5-(1,1- difluoroethyl)-2- pyridyl]phenyl] methyl]- 1,1,4-trioxo-2,3- dihydro-1λ⁶,5- benzothiazepin-7-yl]- 1,3,4-oxadiazol-2-yl]- 2-methyl- propanenitrile (*) | Inter- mediate 26 | 593.3 [M + H]⁺ |
| 110 | 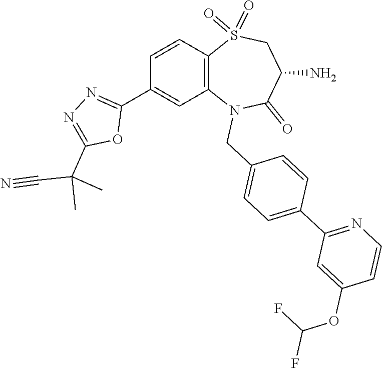 [SMILES: CC(C)(C#N)c1nnc(-c2ccc3c(c2)N(Cc2ccc(-c4cc(OC(F)F)ccn4)cc2)C(=O)[C@@H](N)CS3(=O)=O)o1] | 2-[5-[(3R)-3-amino- 5-[[4-[4- (difluoromethoxy)-2- pyridyl]phenyl] methyl]- 1,1,4-trioxo-2,3- dihydro-1λ⁶,5- benzothiazepin-7-yl]- 1,3,4-oxadiazol-2-yl]- 2-methyl- propanenitrile (*) | Inter- mediate 27 | 595.16 [M + H]⁺ |
| 111 | 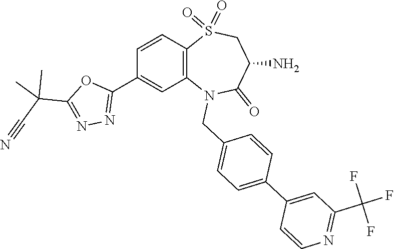 [SMILES: CC(C)(C#N)c1nnc(-c2ccc3c(c2)N(Cc2ccc(-c4ccnc(C(F)(F)F)c4)cc2)C(=O)[C@@H](N)CS3(=O)=O)o1] | 2-[5-[(3R)-3-amino- 1,1,4-trioxo-5-[[4-[2- (trifluoromethyl)-4- pyridyl]phenyl] methyl]- 2,3-dihydro-1λ⁶,5- benzothiazepin-7-yl]- 1,3,4-oxadiazol-2-yl]- 2-methyl- propanenitrile (*) | Inter- mediate 28 | 597.2 [M + H]⁺ |
| 112 | 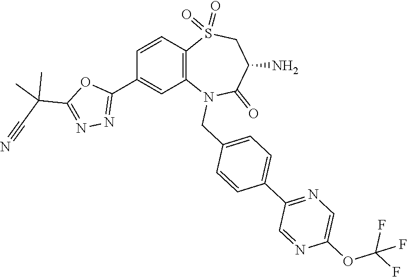 [SMILES: CC(C)(C#N)c1nnc(-c2ccc3c(c2)N(Cc2ccc(-c4cnc(OC(F)(F)F)cn4)cc2)C(=O)[C@@H](N)CS3(=O)=O)o1] | 2-[5-[(3R)-3-amino- 1,1,4-trioxo-5-[[4-[5- (trifluoromethoxy) pyrazin-2- yl]phenyl]methyl]- 2,3-dihydro-1λ⁶,5- benzothiazepin-7-yl]- 1,3,4-oxadiazol-2-yl]- 2-methyl- propanenitrile (*) | Inter- mediate 29 | 614.0 [M + H]⁺ |
| 113 | 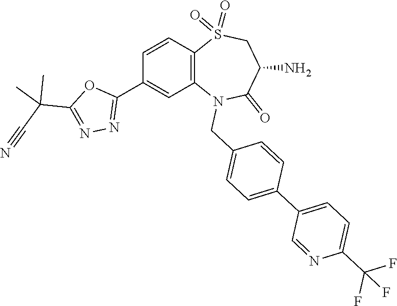 [SMILES: CC(C)(C#N)c1nnc(-c2ccc3c(c2)N(Cc2ccc(-c4ccc(C(F)(F)F)nc4)cc2)C(=O)[C@@H](N)CS3(=O)=O)o1] | 2-[5-[(3R)-3-amino- 1,1,4-trioxo-5-[[4-[6- (trifluoromethyl)-3- pyridyl]phenyl] methyl]- 2,3-dihydro-1λ⁶,5- benzothiazepin-7-yl]- 1,3,4-oxadiazol-2-yl]- 2-methyl- propanenitrile (*) | 5-[4- (bromo- methyl) phenyl]-2- (trifluoro- methyl) pyridine (CAS 960064- 88-4) | 597.2 [M + H]⁺ |
| 114 | 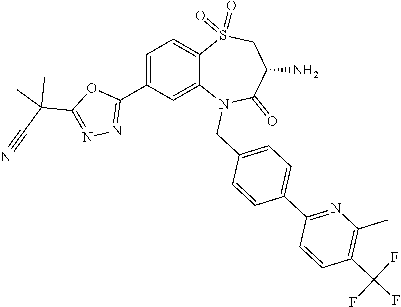 [SMILES: Cc1nc(-c2ccc(CN3C(=O)[C@@H](N)CS(=O)(=O)c4ccc(-c5nnc(C(C)(C)C#N)o5)cc43)cc2)ccc1C(F)(F)F] | 2-[5-[(3R)-3-amino- 5-[[4-[6-methyl-5- (trifluoromethyl)-2- pyridyl]phenyl] methyl]- 1,1,4-trioxo-2,3- dihydro-1λ⁶,5- benzothiazepin-7-yl]- 1,3,4-oxadiazol-2-yl]- 2-methyl- propanenitrile (*) | Inter- mediate 30 | 611.1 [M + H]⁺ |
| 115 | 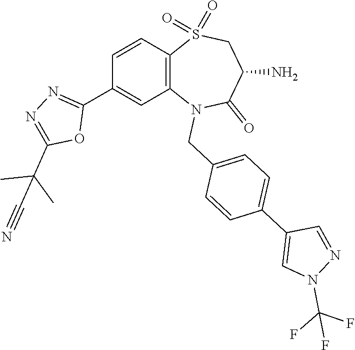 [SMILES: CC(C)(C#N)c1nnc(-c2ccc3c(c2)N(Cc2ccc(-c4cnn(C(F)(F)F)c4)cc2)C(=O)[C@@H](N)CS3(=O)=O)o1] | 2-[5-[(3R)-3-amino- 1,1,4-trioxo-5-[[4-[1- (trifluoromethyl) pyrazol-4- yl]phenyl]methyl]- 2,3-dihydro-1λ⁶,5- benzothiazepin-7-yl]- 1,3,4-oxadiazol-2-yl]- 2-methyl- propanenitrile (*) | Inter- mediate 31 | 586.3 [M + H]⁺ |
| 116 | 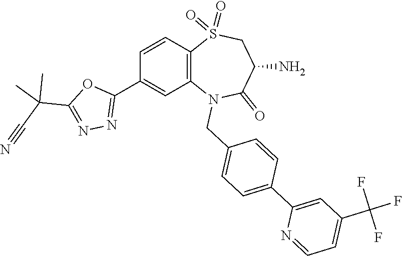 [SMILES: CC(C)(C#N)c1nnc(-c2ccc3c(c2)N(Cc2ccc(-c4cc(C(F)(F)F)ccn4)cc2)C(=O)[C@@H](N)CS3(=O)=O)o1] | 2-[5-[(3R)-3-amino- 1,1,4-trioxo-5-[[4-[4- (trifluoromethyl)-2- pyridyl]phenyl] methyl]- 2,3-dihydro-1λ⁶,5- benzothiazepin-7-yl]- 1,3,4-oxadiazol-2-yl]- 2-methyl- propanenitrile (*) | Inter- mediate 32 | 597.3 [M + H]⁺ |
| 117 | 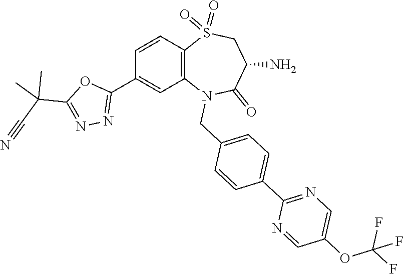 [SMILES: CC(C)(C#N)c1nnc(-c2ccc3c(c2)N(Cc2ccc(-c4ncc(OC(F)(F)F)cn4)cc2)C(=O)[C@@H](N)CS3(=O)=O)o1] | 2-[5-[(3R)-3-amino- 1,1,4-trioxo-5-[[4-[5- (trifluoromethoxy) pyrimidin-2- yl]phenyl]methyl]- 2,3-dihydro-1λ⁶,5- benzothiazepin-7-yl]- 1,3,4-oxadiazol-2-yl]- 2-methyl- propanenitrile (*) | Inter- mediate 33 | 614.1 [M + H]⁺ |
| 118 | 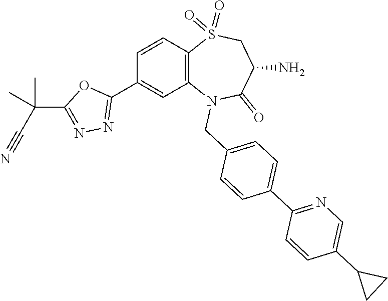 [SMILES: CC(C)(C#N)c1nnc(-c2ccc3c(c2)N(Cc2ccc(-c4ccc(C5CC5)cn4)cc2)C(=O)[C@@H](N)CS3(=O)=O)o1] | 2-[5-[(3R)-3-amino- 5-[[4-(5-cyclopropyl- 2-pyridyl) phenyl]methyl]- 1,1,4-trioxo-2,3- dihydro-1λ⁶,5- benzothiazepin-7-yl]- 1,3,4-oxadiazol-2-yl]- 2-methyl- propanenitrile (*) | Inter- mediate 34 | 569.1 [M + H]⁺ |
| 119 | 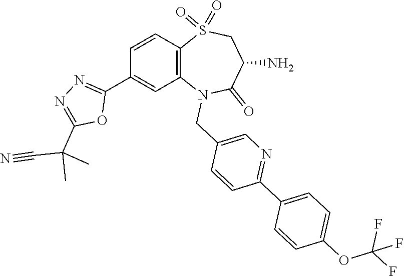 [SMILES: CC(C)(C#N)c1nnc(-c2ccc3c(c2)N(Cc2ccc(-c4ccc(OC(F)(F)F)cc4)nc2)C(=O)[C@@H](N)CS3(=O)=O)o1] | 2-[5-[(3R)-3-amino- 1,1,4-trioxo-5-[[6-[4- (trifluoromethoxy) phenyl]-3- pyridyl]methyl]-2,3- dihydro-1λ⁶,5- benzothiazepin-7-yl]- 1,3,4-oxadiazol-2-yl]- 2-methyl- propanenitrile (*) | 5-(Chloro- methyl)- 2-[4- (trifluoro- methoxy) phenyl] pyridine (CAS 851069- 98-2) | 613.2 [M + H]⁺ |
| 120 | 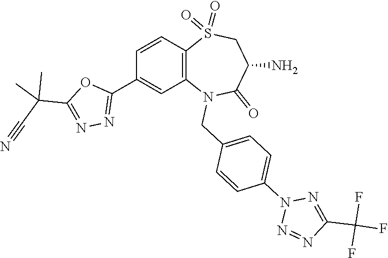 [SMILES: CC(C)(C#N)c1nnc(-c2ccc3c(c2)N(Cc2ccc(-n4nnc(C(F)(F)F)n4)cc2)C(=O)[C@@H](N)CS3(=O)=O)o1] | 2-[5-[(3R)-3-amino- 1,1,4-trioxo-5-[[4-[5- (trifluoromethyl) tetrazol-2- yl]phenyl]methyl]- 2,3-dihydro-1λ⁶,5- benzothiazepin-7-yl]- 1,3,4-oxadiazol-2-yl]- 2-methyl- propanenitrile (*) | Inter- mediate 35 | 588.2 [M + H]⁺ |
| 121 | 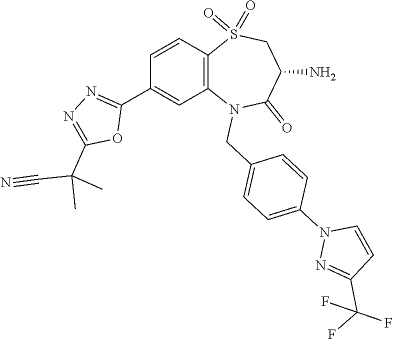 [SMILES: CC(C)(C#N)c1nnc(-c2ccc3c(c2)N(Cc2ccc(-n4ccc(C(F)(F)F)n4)cc2)C(=O)[C@@H](N)CS3(=O)=O)o1] | 2-[5-[(3R)-3-amino- 1,1,4-trioxo-5-[[4-[3- (trifluoromethyl) pyrazol-1- yl]phenyl]methyl]- 2,3-dihydro-1λ⁶,5- benzothiazepin-7-yl]- 1,3,4-oxadiazol-2-yl]- 2-methyl- propanenitrile | 1-[4- (Chloro- methyl) phenyl]-3- (trifluoro- methyl)-1H- pyrazole (CAS 1284983- 51-2) | 586.3 [M + H]⁺ |
| 123 | 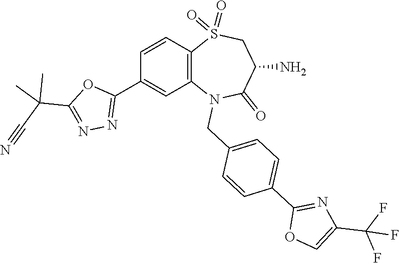 [SMILES: CC(C)(C#N)c1nnc(-c2ccc3c(c2)N(Cc2ccc(-c4nc(C(F)(F)F)co4)cc2)C(=O)[C@@H](N)CS3(=O)=O)o1] | 2-[5-[(3R)-3-amino- 1,1,4-trioxo-5-[[4-[4- (trifluoromethyl) oxazol-2-yl]phenyl] methyl]-2,3- dihydro-1λ⁶,5- benzothiazepin-7-yl]- 1,3,4-oxadiazol-2-yl]- 2-methyl- propanenitrile (*) | Inter- mediate 37 | 587.0 [M + H]⁺ |
| 124 | 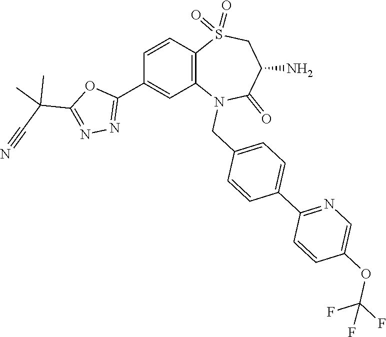 [SMILES: CC(C)(C#N)c1nnc(-c2ccc3c(c2)N(Cc2ccc(-c4ccc(OC(F)(F)F)cn4)cc2)C(=O)[C@@H](N)CS3(=O)=O)o1] | 2-[5-[(3R)-3-amino- 1,1,4-trioxo-5-[[4-[5- (trifluoromethoxy)-2- pyridyl]phenyl] methyl]- 2,3-dihydro-1λ⁶,5- benzothiazepin-7-yl]- 1,3,4-oxadiazol-2-yl]- 2-methyl- propanenitrile (*) | Inter- mediate 38 | 613.3 [M + H]⁺ |
| 125 | 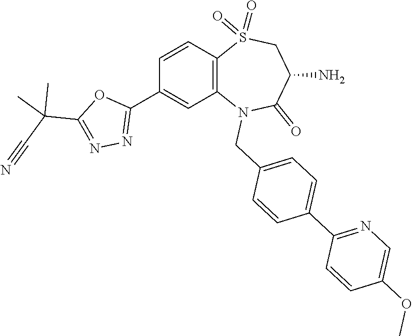 [SMILES: COc1ccc(-c2ccc(CN3C(=O)[C@@H](N)CS(=O)(=O)c4ccc(-c5nnc(C(C)(C)C#N)o5)cc43)cc2)nc1] | 2-[5-[(3R)-3-amino- 5-[[4-(5-methoxy-2- pyridyl)phenyl] methyl]- 1,1,4-trioxo-2,3- dihydro-1λ⁶,5- benzothiazepin-7-yl]- 1,3,4-oxadiazol-2-yl]- 2-methyl- propanenitrile (*) | 2-[4- (bromo- methyl) phenyl]-5- methoxy- pyridine (CAS 1160430- 89-6) | 559.3 [M + H]⁺ |
| 126 | 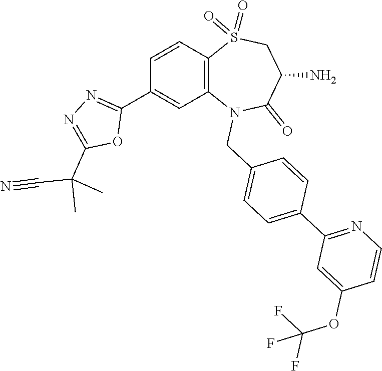 [SMILES: CC(C)(C#N)c1nnc(-c2ccc3c(c2)N(Cc2ccc(-c4cc(OC(F)(F)F)ccn4)cc2)C(=O)[C@@H](N)CS3(=O)=O)o1] | 2-[5-[(3R)-3-amino- 1,1,4-trioxo-5-[[4-[4- (trifluoromethoxy)-2- pyridyl]phenyl] methyl]- 2,3-dihydro-1λ⁶,5- benzothiazepin-7-yl]- 1,3,4-oxadiazol-2-yl]- 2-methyl- propanenitrile (*) | Inter- mediate 39 | 613.2 [M + H]⁺ |
| 127 | 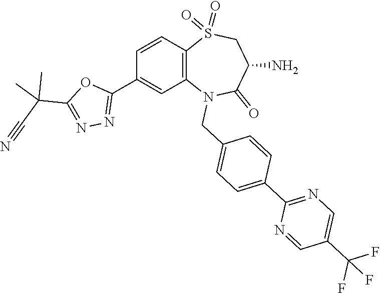 [SMILES: CC(C)(C#N)c1nnc(-c2ccc3c(c2)N(Cc2ccc(-c4ncc(C(F)(F)F)cn4)cc2)C(=O)[C@@H](N)CS3(=O)=O)o1] | 2-[5-[(3R)-3-amino- 1,1,4-trioxo-5-[[4-[5- (trifluoromethyl) pyrimidin-2- yl]phenyl]methyl]- 2,3-dihydro-1λ⁶,5- benzothiazepin-7-yl]- 1,3,4-oxadiazol-2-yl]- 2-methyl- propanenitrile (*) | Inter- mediate 40 | 598.3 [M + H]⁺ |
| 129 | 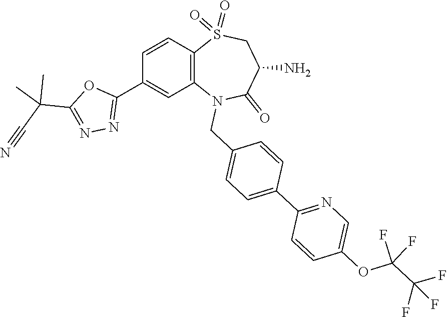 [SMILES: CC(C)(C#N)c1nnc(-c2ccc3c(c2)N(Cc2ccc(-c4ccc(OC(F)(F)C(F)(F)F)cn4)cc2)C(=O)[C@@H](N)CS3(=O)=O)o1] | 2-[5-[(3R)-3-amino- 1,1,4-trioxo-5-[[4-[5- (1,1,2,2,2- pentafluoroethoxy)-2- pyridyl]phenyl] methyl]- 2,3-dihydro-1λ⁶,5- benzothiazepin-7-yl]- 1,3,4-oxadiazol-2-yl]- 2-methyl- propanenitrile (*) | Inter- mediate 42 | 663.3 [M + H]⁺ |
| 135 | 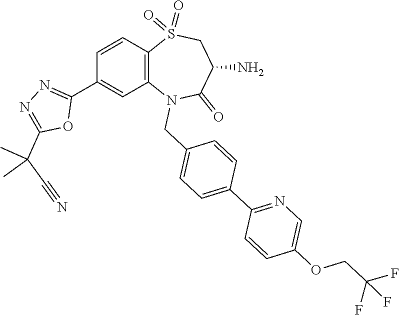 [SMILES: CC(C)(C#N)c1nnc(-c2ccc3c(c2)N(Cc2ccc(-c4ccc(OCC(F)(F)F)cn4)cc2)C(=O)[C@@H](N)CS3(=O)=O)o1] | 2-[5-[(3R)-3-amino- 1,1,4-trioxo-5-[[4-[5- (2,2,2- trifluoroethoxy)-2- pyridyl]phenyl] methyl]- 2,3-dihydro-1λ⁶,5- benzothiazepin-7-yl]- 1,3,4-oxadiazol-2-yl]- 2-methyl- propanenitrile (*) | Inter- mediate 50 | 627.2 [M + H]⁺ |
| 137 | 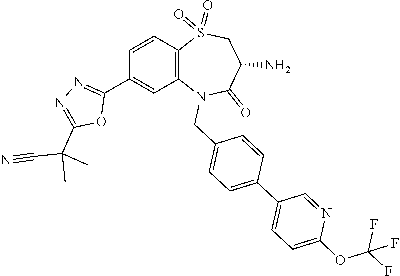 [SMILES: CC(C)(C#N)c1nnc(-c2ccc3c(c2)N(Cc2ccc(-c4ccc(OC(F)(F)F)nc4)cc2)C(=O)[C@@H](N)CS3(=O)=O)o1] | 2-[5-[(3R)-3-amino- 1,1,4-trioxo-5-[[4-[6- (trifluoromethoxy)-3- pyridyl]phenyl] methyl]- 2,3-dihydro-1λ⁶,5- benzothiazepin-7-yl]- 1,3,4-oxadiazol-2-yl]- 2-methyl- propanenitrile (*) | Inter- mediate 52 | 613.2 [M + H]⁺ |
| 138 | 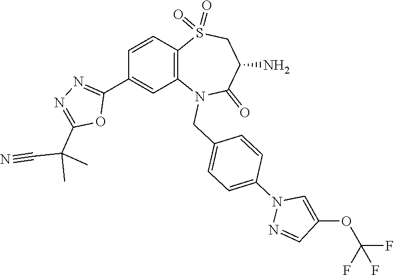 [SMILES: CC(C)(C#N)c1nnc(-c2ccc3c(c2)N(Cc2ccc(-n4cc(OC(F)(F)F)cn4)cc2)C(=O)[C@@H](N)CS3(=O)=O)o1] | 2-[5-[(3R)-3-amino- 1,1,4-trioxo-5-[[4-[4- (trifluoromethoxy) pyrazol-1- yl]phenyl]methyl]- 2,3-dihydro-1λ⁶,5- benzothiazepin-7-yl]- 1,3,4-oxadiazol-2-yl]- 2-methyl- propanenitrile (*) | Inter- mediate 53 | 602.2 [M + H]⁺ |
| 139 | 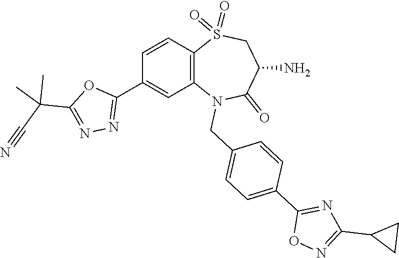 [SMILES: CC(C)(C#N)c1nnc(-c2ccc3c(c2)N(Cc2ccc(-c4nc(C5CC5)no4)cc2)C(=O)[C@@H](N)CS3(=O)=O)o1] | 2-[5-[(3R)-3-amino- 5-[[4-(3-cyclopropyl- 1,2,4-oxadiazol-5- yl)phenyl]methyl]- 1,1,4-trioxo-2,3- dihydro-1λ⁶,5- benzothiazepin-7-yl]- 1,3,4-oxadiazol-2-yl]- 2-methyl- propanenitrile (*) | Inter- mediate 5 | 560.2 [M + H]⁺ |
| 144 | 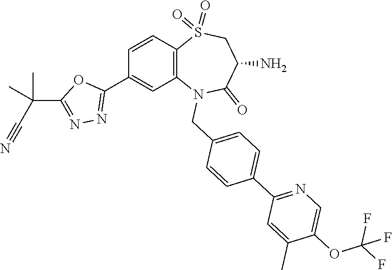 [SMILES: Cc1cc(-c2ccc(CN3C(=O)[C@@H](N)CS(=O)(=O)c4ccc(-c5nnc(C(C)(C)C#N)o5)cc43)cc2)ncc1OC(F)(F)F] | 2-[5-[(3R)-3-amino- 5-[[4-[4-methyl-5- (trifluoromethoxy)-2- pyridyl]phenyl] methyl]- 1,1,4-trioxo-2,3- dihydro-1λ⁶,5- benzothiazepin-7-yl]- 1,3,4-oxadiazol-2-yl]- 2-methyl- propanenitrile (*) | Inter- mediate 59 | 627.2 [M + H]⁺ |
| 145 | 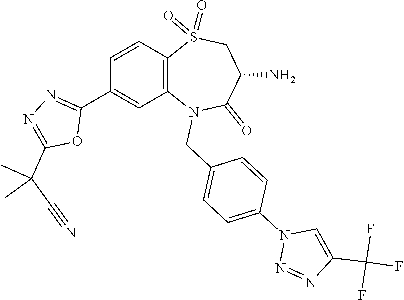 [SMILES: CC(C)(C#N)c1nnc(-c2ccc3c(c2)N(Cc2ccc(-n4cc(C(F)(F)F)nn4)cc2)C(=O)[C@@H](N)CS3(=O)=O)o1] | 2-[5-[(3R)-3-amino- 1,1,4-trioxo-5-[[4-[4- (trifluoromethyl) triazol-1- yl]phenyl]methyl]- 2,3-dihydro-1λ⁶,5- benzothiazepin-7-yl]- 1,3,4-oxadiazol-2-yl]- 2-methyl- propanenitrile (*) | Inter- mediate 60 | 587.5 [M + H]⁺ |
| 148 | 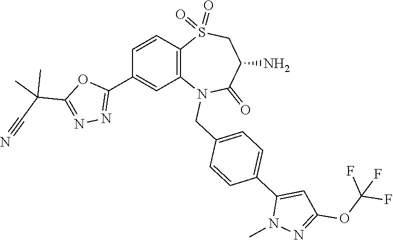 [SMILES: Cn1nc(OC(F)(F)F)cc1-c1ccc(CN2C(=O)[C@@H](N)CS(=O)(=O)c3ccc(-c4nnc(C(C)(C)C#N)o4)cc32)cc1] | 2-[5-[(3R)-3-amino- 5-[[4-[2-methyl-5- (trifluoromethoxy) pyrazol-3-yl] phenyl]methyl]-1,1,4- trioxo-2,3- dihydro-1λ⁶,5- benzothiazepin-7-yl]- 1,3,4-oxadiazol-2-yl]- 2-methyl- propanenitrile (*) | Inter- mediate 63 | 616.2 [M + H]⁺ |

(*) as hydrochloride salt

### Example 48

### (3R)-3-amino-7-[5-(2-methyloxetan-2-yl)-1,3,4-oxadiazol-2-yl]-1,1-dioxo-5-[[4-[4-(trifluoromethyl)phenyl]phenyl]methyl]-2,3-dihydro-1lambda6,5-benzothiazepin-4-one

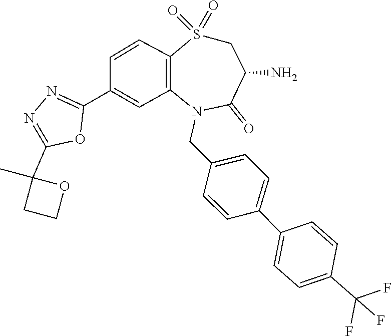 [SMILES: CC1(c2nnc(-c3ccc4c(c3)N(Cc3ccc(-c5ccc(C(F)(F)F)cc5)cc3)C(=O)[C@@H](N)CS4(=O)=O)o2)CCO1]

### Step a) tert-butyl N-[(3R)-7-[[(2-methyloxetane-2-carbonyl)amino]carbamoyl]-1,1,4-trioxo-3,5-dihydro-2H-1lambda6,5-benzothiazepin-3-yl]carbamate

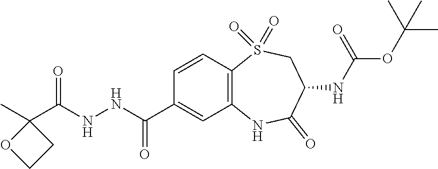 [SMILES: CC(C)(C)OC(=O)N[C@H]1CS(=O)(=O)c2ccc(C(=O)NNC(=O)C3(C)CCO3)cc2NC1=O]

The title compound was prepared from tert-butyl N-[(3R)-7-(hydrazinecarbonyl)-1,1,4-trioxo-3,5-dihydro-2H-1λ⁶,5-benzothiazepin-3-yl]carbamate (90 mg, 0.234 mmol, 1.0 eq, Example 32, step b) and 2-methyl-2-oxetanecarboxylic acid (32.6 mg, 0.28 mmol, 1.2 eq, CAS 1305207-92-4) in analogy to general procedure 4a and was obtained as off-white powder (60 mg, 53%). MS (ESI): 481.2 [M+H]⁺

### Step b) tert-butyl N-[(3R)-7-[5-(2-methyloxetan-2-yl)-1,3,4-oxadiazol-2-yl]-1,1,4-trioxo-3,5-dihydro-2H-1lambda6,5-benzothiazepin-3-yl]carbamate

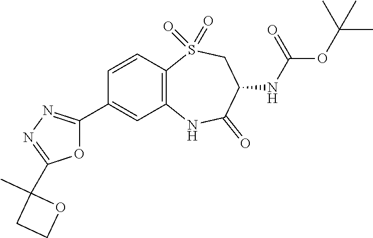 [SMILES: CC(C)(C)OC(=O)N[C@H]1CS(=O)(=O)c2ccc(-c3nnc(C4(C)CCO4)o3)cc2NC1=O]

The title compound was prepared from tert-butyl N-[(3R)-7-[[(2-methyloxetane-2-carbonyl)amino]carbamoyl]-1,1,4-trioxo-3,5-dihydro-2H-1lambda6,5-benzothiazepin-3-yl]carbamate (60 mg, 0.106 mmol) in analogy to general procedure 5b and was obtained as white powder (25 mg, 43%). MS (ESI): 463.1 [M+H]⁺

### Step c) tert-butyl N-[(3R)-7-[5-(2-methyloxetan-2-yl)-1,3,4-oxadiazol-2-yl]-1,1,4-trioxo-5-[[4-[4-(trifluoromethyl)phenyl]phenyl]methyl]-2,3-dihydro-1lambda6,5-benzothiazepin-3-yl]carbamate

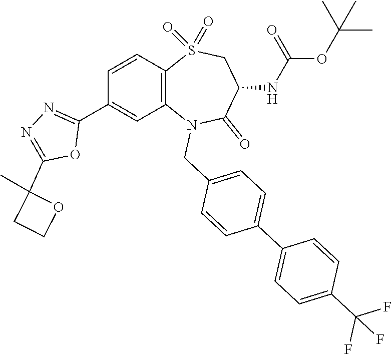 [SMILES: CC(C)(C)OC(=O)N[C@H]1CS(=O)(=O)c2ccc(-c3nnc(C4(C)CCO4)o3)cc2N(Cc2ccc(-c3ccc(C(F)(F)F)cc3)cc2)C1=O]

The title compound was prepared from tert-butyl N-[(3R)-7-[5-(2-methyloxetan-2-yl)-1,3,4-oxadiazol-2-yl]-1,1,4-trioxo-3,5-dihydro-2H-1lambda6,5-benzothiazepin-3-yl]carbamate (25 mg, 0.054 mmol, 1.0 eq) and 4-(bromomethyl)-4′-(trifluoromethyl)-1,1′-biphenyl (22 mg, 0.070 μmol, 1.3 eq, CAS 613241-14-8) in analogy to general procedure 1a and was obtained as white powder (17 mg, 44%). MS (ESI): 643.2 [M-isobutene+H]⁺

### Step d) (3R)-3-amino-7-[5-(2-methyloxetan-2-yl)-1,3,4-oxadiazol-2-yl]-1,1-dioxo-5-[[4-[4-(trifluoromethyl)phenyl]phenyl]methyl]-2,3-dihydro-1lambda6,5-benzothiazepin-4-one

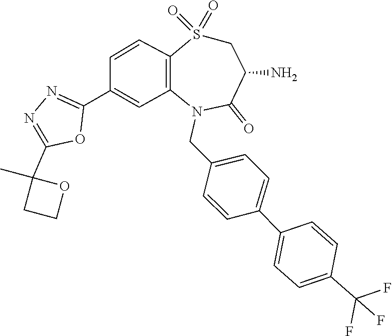 [SMILES: CC1(c2nnc(-c3ccc4c(c3)N(Cc3ccc(-c5ccc(C(F)(F)F)cc5)cc3)C(=O)[C@@H](N)CS4(=O)=O)o2)CCO1]

The title compound was prepared from tert-butyl N-[(3R)-7-[5-(2-methyloxetan-2-yl)-1,3,4-oxadiazol-2-yl]-1,1,4-trioxo-5-[[4-[4-(trifluoromethyl)phenyl]phenyl]methyl]-2,3-dihydro-1lambda6,5-benzothiazepin-3-yl]carbamate (24.8 mg, 0.035 mmol) in analogy to general procedure 11a and was obtained as white powder (8.5 mg, 40%). MS (ESI): 599.2 [M+H]⁺

The examples of the following table were prepared in analogy to Example 48, using the appropriate benzyl bromide building block.

| | | | | MS, |
|---|---|---|---|---|
| | | | Building | ESI: |
| Ex. | Structure | Systematic Name | Block | m/z |
| 149 | 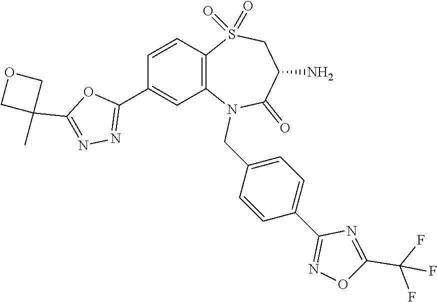 [SMILES: CC1(c2nnc(-c3ccc4c(c3)N(Cc3ccc(-c5noc(C(F)(F)F)n5)cc3)C(=O)[C@@H](N)CS4(=O)=O)o2)COC1] | (3R)-3-amino-7-[5- (3-methyloxetan-3- yl)-1,3,4- oxadiazol-2-yl]- 1,1-dioxo-5-[[4-[5- (trifluoromethyl)- 1,2,4-oxadiazol-3- yl]phenyl]methyl]- 2,3-dihydro-1λ⁶,5- benzothiazepin-4- one | 3-[4- (bromomethyl) phenyl]-5- (trifluoromethyl)- 1,2,4- oxadiazole (CAS 2093101-98-3) | 591.2 [M + H]⁺ |
| 150 | 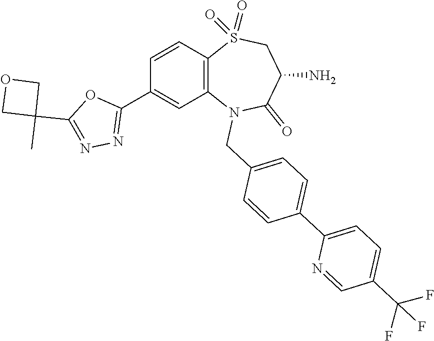 [SMILES: CC1(c2nnc(-c3ccc4c(c3)N(Cc3ccc(-c5ccc(C(F)(F)F)cn5)cc3)C(=O)[C@@H](N)CS4(=O)=O)o2)COC1] | (3R)-3-amino-7-[5- (3-methyloxetan-3- yl)-1,3,4- oxadiazol-2-yl]- 1,1-dioxo-5-[[4-[5- (trifluoromethyl)- 2- pyridyl]phenyl] methyl]-2,3- dihydro-1λ⁶,5- benzothiazepin- 4-one | 2-[4- (bromomethyl) phenyl]-5- (trifluoromethyl) pyridine (CAS 1056641-21-4) | 600.3 [M + H]⁺ |

The examples of the following table were prepared in analogy to Example 48, using the appropriate carboxylic acid building block.

| | | | Building | MS, ESI: |
|---|---|---|---|---|
| Ex. | Structure | Systematic Name | Block | m/z |
| 151 | 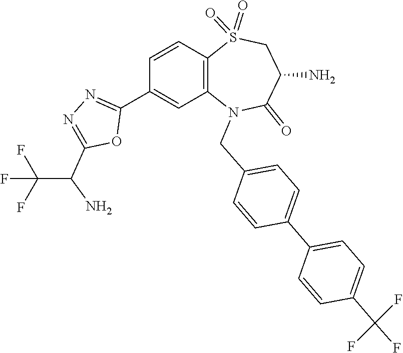 [SMILES: NC(c1nnc(-c2ccc3c(c2)N(Cc2ccc(-c4ccc(C(F)(F)F)cc4)cc2)C(=O)[C@@H](N)CS3(=O)=O)o1)C(F)(F)F] | (3R)-3-amino-7-[5- (1-amino-2,2,2- trifluoro-ethyl)- 1,3,4-oxadiazol-2- yl]-1,1-dioxo-5- [[4-[4- (trifluoromethyl) phenyl]phenyl] methyl]-2,3- dihydro-1λ⁶,5- benzothiazepin-4- one (*) | 2-((tert- butoxycarbonyl) amino)-3,3,3- trifluoropropanoic acid (CAS 188030-43-5) | 626.2 [M + H]⁺ |
| 152 | 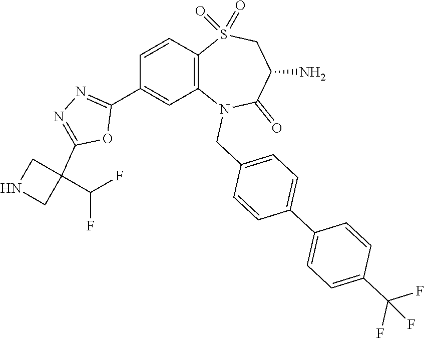 [SMILES: N[C@H]1CS(=O)(=O)c2ccc(-c3nnc(C4(C(F)F)CNC4)o3)cc2N(Cc2ccc(-c3ccc(C(F)(F)F)cc3)cc2)C1=O] | (3R)-3-amino-7-[5- [3- (difluoromethyl) azetidin-3-yl]-1,3,4- oxadiazol-2-yl]- 1,1-dioxo-5-[[4-[4- (trifluoromethyl) phenyl]phenyl] methyl]-2,3- dihydro-1λ⁶,5- benzothiazepin-4- one (*) | 1-[(tert- butoxy)carbonyl]- 3- (difluoromethyl) azetidine-3- carboxylic acid (CAS 1784562-69-1) | 678.3 [M + H]⁺ |

(*) as hydrochloride salt

### Example 49

### (3R)-3-amino-7-[5-(3-fluoro-1-methyl-3-piperidyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

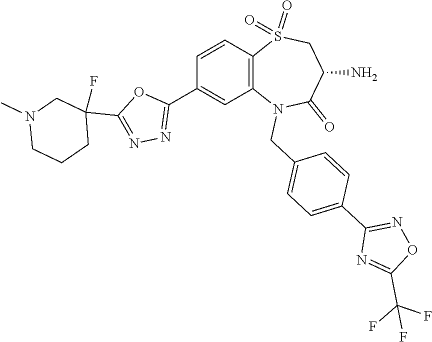 [SMILES: CN1CCCC(F)(c2nnc(-c3ccc4c(c3)N(Cc3ccc(-c5noc(C(F)(F)F)n5)cc3)C(=O)[C@@H](N)CS4(=O)=O)o2)C1]

### Step a) benzyl 3-[[[(3R)-3-(tert-butoxycarbonylamino)-4-oxo-3,5-dihydro-2H-1,5-benzothiazepine-7-carbonyl]amino]carbamoyl]-3-fluoro-piperidine-1-carboxylate

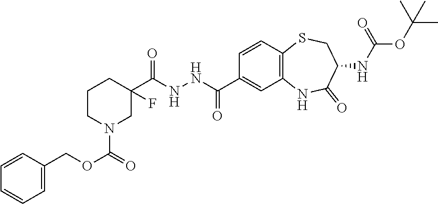 [SMILES: CC(C)(C)OC(=O)N[C@H]1CSc2ccc(C(=O)NNC(=O)C3(F)CCCN(C(=O)OCc4ccccc4)C3)cc2NC1=O]

The title compound was prepared from tert-butyl N-[(3R)-7-(hydrazinecarbonyl)-4-oxo-3,5-dihydro-2H-1,5-benzothiazepin-3-yl]carbamate (400 mg, 1.14 mmol, 1.0 eq, Example 19, step b) and 1-(phenylmethyl) 3-fluoro-1,3-piperidinedicarboxylate (351 mg, 1.25 mmol, 1.1 eq, CAS 1363166-38-4) in analogy to general procedure 4a and was obtained as white solid (680 mg, 97%). MS (ESI): 516.2 [M-Boc+H]⁺

### Step b) benzyl 3-[5-[(3R)-3-(tert-butoxycarbonylamino)-4-oxo-3,5-dihydro-2H-1,5-benzothiazepin-7-yl]-1,3,4-oxadiazol-2-yl]-3-fluoro-piperidine-1-carboxylate

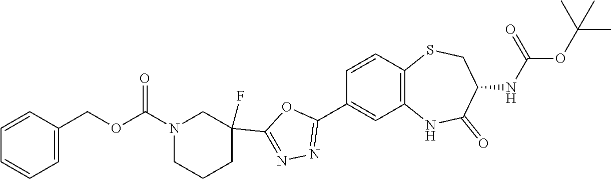 [SMILES: CC(C)(C)OC(=O)N[C@H]1CSc2ccc(-c3nnc(C4(F)CCCN(C(=O)OCc5ccccc5)C4)o3)cc2NC1=O]

The title compound was prepared from benzyl 3-[[[(3R)-3-(tert-butoxycarbonylamino)-4-oxo-3,5-dihydro-2H-1,5-benzothiazepine-7-carbonyl]amino]carbamoyl]-3-fluoro-piperidine-1-carboxylate (680 mg, 1.1 mmol, 1.0 eq) in analogy to general procedure 5b and was obtained as white solid (505 mg, 77%). MS (ESI): 542.1 [M-isobutene+H]⁺

### Step c) benzyl 3-[5-[(3R)-3-(tert-butoxycarbonylamino)-4-oxo-3,5-dihydro-2H-1,5-benzothiazepin-7yl]-1,3,4-oxadiazol-2-yl]-3-fluoro-piperidine-1-carboxylate

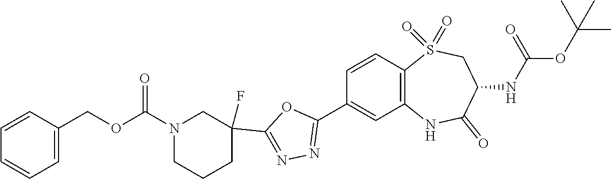 [SMILES: CC(C)(C)OC(=O)N[C@H]1CS(=O)(=O)c2ccc(-c3nnc(C4(F)CCCN(C(=O)OCc5ccccc5)C4)o3)cc2NC1=O]

The title compound was prepared from benzyl 3-[5-[(3R)-3-(tert-butoxycarbonylamino)-4-oxo-3,5-dihydro-2H-1,5-benzothiazepin-7-yl]-1,3,4-oxadiazol-2-yl]-3-fluoro-piperidine-1-carboxylate (180 mg, 0.295 mmol, 1 eq) in analogy to general procedure 10 and was obtained as white solid (180 mg, 97%). MS (ESI): 628.4 [M+H]⁺

### Step d) benzyl 3-[5-[(3R)-3-(tert-butoxycarbonylamino)-1,1,4-trioxo-5-[[4-[5-(trifluoromethyl)-1,2,4-oxadiazol-3-yl]phenyl]methyl]-2,3-dihydro-1λ⁶, 5-benzothiazepin-7-yl]-1,3,4-oxadiazol-2-yl]-3-fluoro-piperidine-1-carboxylate

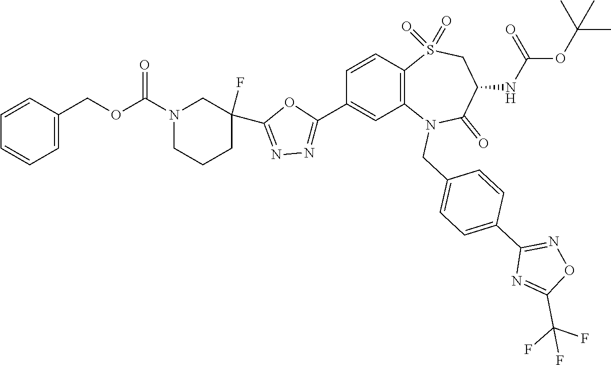 [SMILES: CC(C)(C)OC(=O)N[C@H]1CS(=O)(=O)c2ccc(-c3nnc(C4(F)CCCN(C(=O)OCc5ccccc5)C4)o3)cc2N(Cc2ccc(-c3noc(C(F)(F)F)n3)cc2)C1=O]

The title compound was prepared from benzyl 3-[5-[(3R)-3-(tert-butoxycarbonylamino)-4-oxo-3,5-dihydro-2H-1,5-benzothiazepin-7-yl]-1,3,4-oxadiazol-2-yl]-3-fluoro-piperidine-1-carboxylate (80 mg, 0.127 mmol, 1.0 eq) and 3-[4-(bromomethyl)phenyl]-5-(trifluoromethyl)-1,2,4-oxadiazole (50.7 mg, 0.165 mmol, 1.3 eq, CAS 2093101-98-3) in analogy to general procedure 1a and was obtained as white solid (89.6 mg, 81%). MS (ESI): 800.4 [M-isobutene+H]⁺

### Step e) tert-butyl N-[(3R)-7-[5-(3-fluoro-3-piperidyl)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶, 5-benzothiazepin-3-yl]carbamate

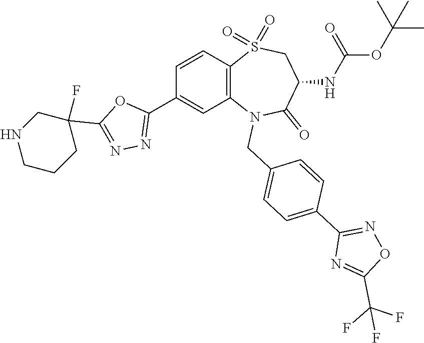 [SMILES: CC(C)(C)OC(=O)N[C@H]1CS(=O)(=O)c2ccc(-c3nnc(C4(F)CCCNC4)o3)cc2N(Cc2ccc(-c3noc(C(F)(F)F)n3)cc2)C1=O]

To a solution benzyl 3-[5-[(3R)-3-(tert-butoxycarbonylamino)-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]-3-fluoro-piperidine-1-carboxylate (89.6 mg, 0.103 mmol, 1.0 eq) in MeOH (1.7 mL) and THF (0.5 mL) was added Pd/C (22 mg) under inert atmosphere. The reaction mixture was stirred under hydrogen atmosphere for 5 hours. The mixture was filtered through a plug of celite, washing with THF and MeOH. The filtrate was concentrated to afford tert-butyl N-[(3R)-7-[5-(3-fluoro-3-piperidyl)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (81.7 mg, 55%) as light yellow solid. MS (ESI): 722.6 [M+H]⁺

### Step f) tert-butyl N-[(3R)-7-[5-(3-fluoro-1-methyl-3-piperidyl)-], 3,4-oxadiazol-2-yl]-1,1,4-trioxo-5-[[[4-[5-(trifluoromethyl)-1,2,4-oxadiazol-3-yl]phenyl]methyl]-2,3-dihydro-1λ⁶, 5-benzothiazepin-3-yl]carbamate

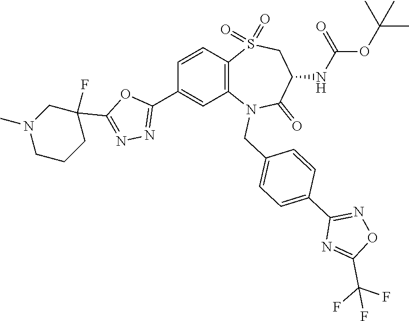 [SMILES: CN1CCCC(F)(c2nnc(-c3ccc4c(c3)N(Cc3ccc(-c5noc(C(F)(F)F)n5)cc3)C(=O)[C@@H](NC(=O)OC(C)(C)C)CS4(=O)=O)o2)C1]

To a solution tert-butyl N-[(3R)-7-[5-(3-fluoro-3-piperidyl)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (81.7 mg, 0.057 mmol, 1.0 eq) in MeOH (0.57 mL) was added Formalin (37% aq., 56 uL, 0.57 mmol, 10 eq) and sodium triacetoxyborohydride (120 mg, 0.57 mmol, 10 eq). The mixture was stirred at RT for two hours. The reaction was poured on water and extracted with DCM (3×). The combined organic phase was dried over magnesium sulfate, filtered and concentrated under reduced pressure. The remaining crude was purified using flash column chromatography on silica gel (0-100% EtOAc in heptane) to afford tert-butyl N-[(3R)-7-[5-(3-fluoro-1-methyl-3-piperidyl)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (15.9 mg, 38%) as white solid. MS (ESI): 736.6 [M+H]⁺

### Step g) (3R)-3-amino-7-[5-(3-fluoro-1-methyl-3-piperidyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶ 5-benzothiazepin-4-one

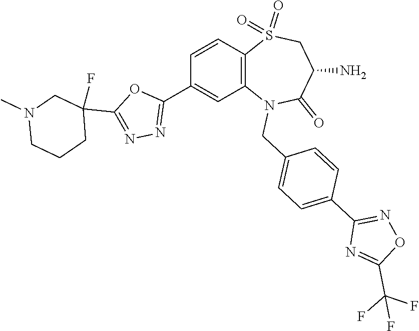 [SMILES: CN1CCCC(F)(c2nnc(-c3ccc4c(c3)N(Cc3ccc(-c5noc(C(F)(F)F)n5)cc3)C(=O)[C@@H](N)CS4(=O)=O)o2)C1]

The title compound was prepared from tert-butyl N-[(3R)-7-[5-(3-fluoro-1-methyl-3-piperidyl)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (15.9 mg, 0.022 mmol) in analogy to general procedure 11a and was obtained as off-white solid, as hydrochloride salt (9.8 mg, 68%). MS (ESI): 636.3 [M+H]⁺

The examples of the following table were prepared in analogy to Example 49, using the appropriate benzyl bromide building block.

| | | | | MS, |
|---|---|---|---|---|
| | | | Building | ESI: |
| Ex. | Structure | Systematic Name | Block | m/z |
| 50 | 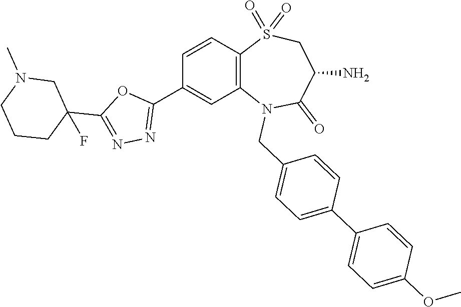 [SMILES: COc1ccc(-c2ccc(CN3C(=O)[C@@H](N)CS(=O)(=O)c4ccc(-c5nnc(C6(F)CCCN(C)C6)o5)cc43)cc2)cc1] | (3R)-3-amino-7-[5-(3- fluoro-1-methyl-3- piperidyl)-1,3,4- oxadiazol-2-yl]-5-[[4-(4- methoxyphenyl)phenyl] methyl]-1,1-dioxo-2,3- dihydro-1λ⁶,5- benzothiazepin-4-one (*) | 4- (Bromomethyl)- 4′-methoxy- 1,1′-biphenyl (CAS 20854-61-9) | 606.2 [M + H]⁺ |
| 51 | 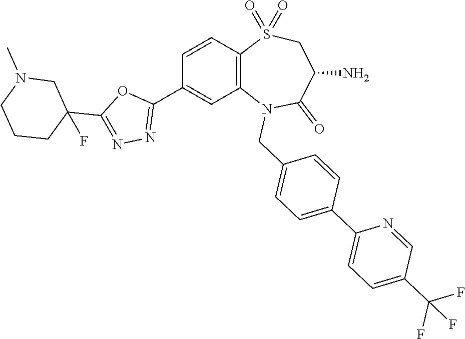 [SMILES: CN1CCCC(F)(c2nnc(-c3ccc4c(c3)N(Cc3ccc(-c5ccc(C(F)(F)F)cn5)cc3)C(=O)[C@@H](N)CS4(=O)=O)o2)C1] | (3R)-3-amino-7-[5-(3- fluoro-1-methyl-3- piperidyl)-1,3,4- oxadiazol-2-yl]-1,1- dioxo-5-[[4-[5- (trifluoromethyl)-2- pyridyl]phenyl]methyl]- 2,3-dihydro-1λ⁶,5- benzothiazepin-4-one (*) | 2-[4- (bromomethyl) phenyl]-5- (trifluoromethyl) pyridine (CAS 1056641-21-4) | 643.3 [M − H] |

(*) as hydrochloride salt

### Example 52

### (3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

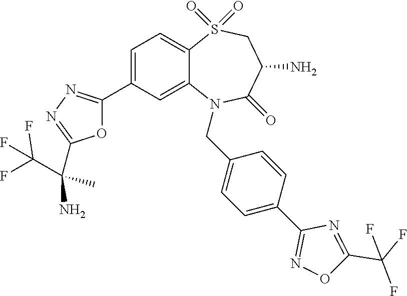 [SMILES: C[C@@](N)(c1nnc(-c2ccc3c(c2)N(Cc2ccc(-c4noc(C(F)(F)F)n4)cc2)C(=O)[C@@H](N)CS3(=O)=O)o1)C(F)(F)F]

### Step a) (2R)-2-(tert-butoxycarbonylamino)-3,3,3-trifluoro-2-methyl-propanoic Acid

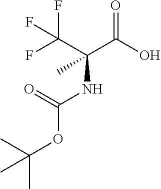 [SMILES: CC(C)(C)OC(=O)N[C@](C)(C(=O)O)C(F)(F)F]

To a solution of (2R)-2-amino-3,3,3-trifluoro-2-methyl-propoionic acid (1 g, 6.37 mmol, 1.0 eq, CAS 102210-02-6) in THF (20 ml) at RT was added DMAP (233 mg, 1.91 mmol, 0.3 eq) and Di-tert-butyl dicarbonate (1.67 g, 7.64 mmol, 1.2 eq) and the reaction mixture was stirred overnight. The resulting solution was poured into sat. NaHCO₃ and washed twice with EtOAc. The aqueous phase was then acidified with 1N HCl and extracted with EtOAc (3×). The combined organic phases were dried over sodium sulfate, filtered and concentrated to afford (2R)-2-(tert-butoxycarbonylamino)-3,3,3-trifluoro-2-methyl-propanoic acid (1.32 g, 81%) as white solid. MS (ESI): 158.0 [M-Boc+H]⁺

### Step b) tert-butyl N-[(1R)-1-[[[(3R)-3-(tert-butoxycarbonylamino)-4-oxo-3,5-dihydro-2H-1,5-benzothiazepine-7-carbonyl]amino]carbamoyl]-2,2,2-trifluoro-1-methyl-ethyl]carbamate

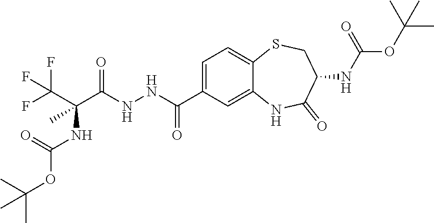 [SMILES: CC(C)(C)OC(=O)N[C@H]1CSc2ccc(C(=O)NNC(=O)[C@@](C)(NC(=O)OC(C)(C)C)C(F)(F)F)cc2NC1=O]

The title compound was prepared from tert-butyl N-[(3R)-7-(hydrazinecarbonyl)-4-oxo-3,5-dihydro-2H-1,5-benzothiazepin-3-yl]carbamate (180 mg, 0.510 mmol, 1.0 eq) and (2R)-2-(tert-butoxycarbonylamino)-3,3,3-trifluoro-2-methyl-propanoic acid (131.37 mg, 0.510 mmol, 1.0 eq, CAS) in analogy to general procedure 4b and was obtained as white solid (88 mg, 29%). MS (ESI): 590.3 [M−H]

### Step c) tert-butyl N-[(1R)-1-[5-[(3R)-3-(tert-butoxycarbonylamino)-4-oxo-3,5-dihydro-2H-1,5-benzothiazepin-7-yl]-1,3,4-oxadiazol-2-yl]-2,2,2-trifluoro-1-methyl-ethyl]carbamate

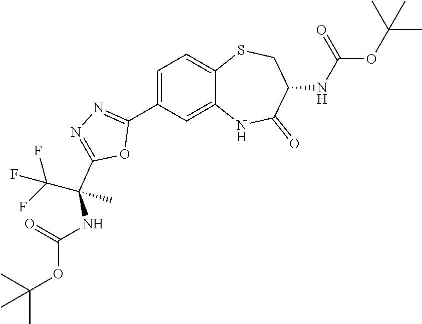 [SMILES: CC(C)(C)OC(=O)N[C@H]1CSc2ccc(-c3nnc([C@@](C)(NC(=O)OC(C)(C)C)C(F)(F)F)o3)cc2NC1=O]

The title compound was prepared from tert-butyl N-[(1R)-1-[[[(3R)-3-(tert-butoxycarbonylamino)-4-oxo-3,5-dihydro-2H-1,5-benzothiazepine-7-carbonyl]amino]carbamoyl]-2,2,2-trifluoro-1-methyl-ethyl]carbamate (750 mg, 0.3 mmol, 1.0 eq) in analogy to general procedure 5a and was obtained as white solid (82 mg, 46%). MS (ESI): 572.3 [M−H]

### Step d) tert-butyl N-[(1R)-1-[5-[(3R)-3-(tert-butoxycarbonylamino)-4-oxo-5-[[4-[5-(trifluoromethyl)-1,2,4-oxadiazol-3-yl]phenyl]methyl]-2,3-dihydro-1,5-benzothiazepin-7-yl]-1,3,4-oxadiazol-2-yl]-2,2,2-trifluoro-1-methyl-ethyl]carbamate

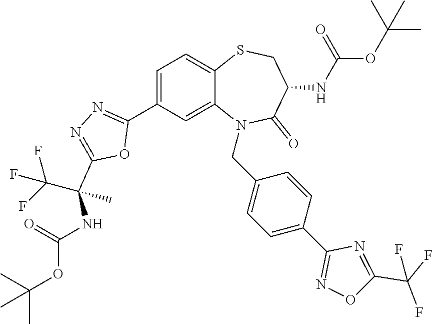 [SMILES: CC(C)(C)OC(=O)N[C@H]1CSc2ccc(-c3nnc([C@@](C)(NC(=O)OC(C)(C)C)C(F)(F)F)o3)cc2N(Cc2ccc(-c3noc(C(F)(F)F)n3)cc2)C1=O]

The title compound was prepared from tert-butyl N-[(1R)-1-[5-[(3R)-3-(tert-butoxycarbonylamino)-4-oxo-3,5-dihydro-2H-1,5-benzothiazepin-7-yl]-1,3,4-oxadiazol-2-yl]-2,2,2-trifluoro-1-methyl-ethyl]carbamate (144 mg, 0.251 mmol, 1.0 eq) and 3-[4-(bromomethyl)phenyl]-5-(trifluoromethyl)-1,2,4-oxadiazole (92.5 mg, 0.301 mmol, 1.2 eq, CAS 2093101-98-3) in analogy to general procedure 1a and was obtained as white solid (153 mg, 76%). MS (ESI): 688.2 [M-Boc+H]⁺

### Step e) tert-butyl N-[(3R)-7-[5-[(1R)-1-(tert-butoxycarbonylamino)-2,2,2-trifluoro-1-methyl-ethyl]-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate

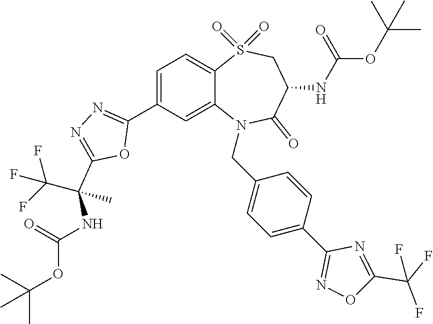 [SMILES: CC(C)(C)OC(=O)N[C@H]1CS(=O)(=O)c2ccc(-c3nnc([C@@](C)(NC(=O)OC(C)(C)C)C(F)(F)F)o3)cc2N(Cc2ccc(-c3noc(C(F)(F)F)n3)cc2)C1=O]

The title compound was prepared from tert-butyl N-[(1R)-1-[5-[(3R)-3-(tert-butoxycarbonylamino)-4-oxo-5-[[4-[5-(trifluoromethyl)-1,2,4-oxadiazol-3-yl]phenyl]methyl]-2,3-dihydro-1,5-benzothiazepin-7-yl]-1,3,4-oxadiazol-2-yl]-2,2,2-trifluoro-1-methyl-ethyl]carbamate (153 mg, 191.31 μmol, 1.0 eq) in analogy to general procedure 10 and was obtained as white solid (122 mg, 77%). MS (ESI): 830.4 [M−H]

### Step f) (3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

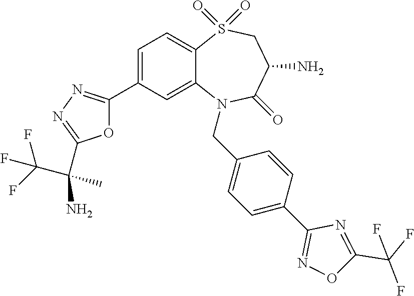 [SMILES: C[C@@](N)(c1nnc(-c2ccc3c(c2)N(Cc2ccc(-c4noc(C(F)(F)F)n4)cc2)C(=O)[C@@H](N)CS3(=O)=O)o1)C(F)(F)F]

The title compound was prepared from tert-butyl N-[(3R)-7-[5-[(1R)-1-(tert-butoxycarbonylamino)-2,2,2-trifluoro-1-methyl-ethyl]-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (40 mg, 0.048 mmol, 1.0 eq) in analogy to general procedure 11a and was obtained as white solid, as hydrochloride salt (31 mg, 97%). MS (ESI): 632.1 [M+H]⁺

The examples of the following table were prepared in analogy to Example 52, using the appropriate carboxylic acid building block.

| | | | | MS, |
|---|---|---|---|---|
| | | | Building | ESI: |
| Ex. | Structure | Systematic Name | Block | m/z |
| 53 | 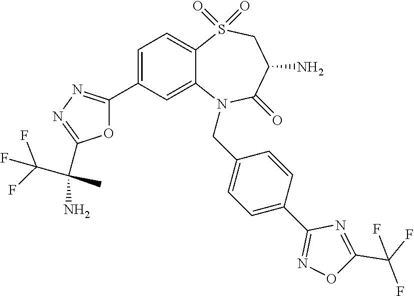 [SMILES: C[C@](N)(c1nnc(-c2ccc3c(c2)N(Cc2ccc(-c4noc(C(F)(F)F)n4)cc2)C(=O)[C@@H](N)CS3(=O)=O)o1)C(F)(F)F] | (3R)-3-amino-7-[5- [(1S)-1-amino- 2,2,2-trifluoro-1- methyl-ethyl]- 1,3,4-oxadiazol-2- yl]-1,1-dioxo-5- [[4-[5- (trifluoromethyl)- 1,2,4-oxadiazol-3- yl]phenyl]methyl]- 2,3-dihydro-1λ⁶,5- benzothiazepin-4- one (*) | (2S)-2- amino-3,3,3- trifluoro-2- methyl- propionic acid (CAS 102210-03-7) | MS (ESI): 632.1 [M + H]⁺ |

(*) as hydrochloride salt

### Example 153

### (3R)-3-amino-7-[5-[(1R)-1-amino-2,2,2-trifluoro-1-methyl-ethyl]-1,3,4-oxadiazol-2-yl]-1,1-dioxo-5-[1[4-5-(trifluoromethoxy)-2-pyridyl]phenyl]methyl]-2,3-dihydro-1λ⁶,5-benzothiazepin-4-one

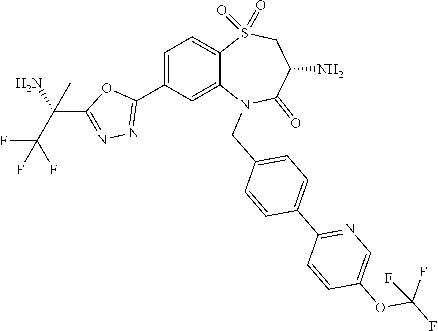 [SMILES: C[C@@](N)(c1nnc(-c2ccc3c(c2)N(Cc2ccc(-c4ccc(OC(F)(F)F)cn4)cc2)C(=O)[C@@H](N)CS3(=O)=O)o1)C(F)(F)F]

### Step a) tert-butyl N-[(3R)-7-[5-[(1R)-1-(tert-butoxycarbonylamino)-2,2,2-trifluoro-1-methyl-ethyl]-1,3,4-oxadiazol-2-yl]-1,1,4-trioxo-3,5-dihydro-2H-1λ⁶,5-benzothiazepin-3-yl]carbamate

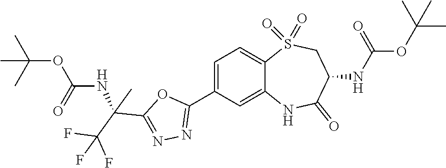 [SMILES: CC(C)(C)OC(=O)N[C@H]1CS(=O)(=O)c2ccc(-c3nnc([C@@](C)(NC(=O)OC(C)(C)C)C(F)(F)F)o3)cc2NC1=O]

The title compound was prepared from tert-butyl N-[(1R)-1-[5-[(3R)-3-(tert-butoxycarbonylamino)-4-oxo-3,5-dihydro-2H-1,5-benzothiazepin-7-yl]-1,3,4-oxadiazol-2-yl]-2,2,2-trifluoro-1-methyl-ethyl]carbamate (142 mg, 247.6 μmol, example 52, step c) in analogy to general procedure 10 and was obtained as white solid (135 mg, 90%) as white solid. MS (ESI): 604.2 [M+H]⁺

### Step b) tert-butyl N-[(3R)-7-[5-[(1R)-1-(tert-butoxycarbonylamino)-2,2,2-trifluoro-1-methyl-ethyl]-1,3,4-oxadiazol-2-yl]-1,1,4-trioxo-5-[[4-[5-(trifluoromethoxy)-2-pyridyl]phenyl]methyl]-2,3-dihydro-1λ⁶, 5-benzothiazepin-3-yl]carbamate

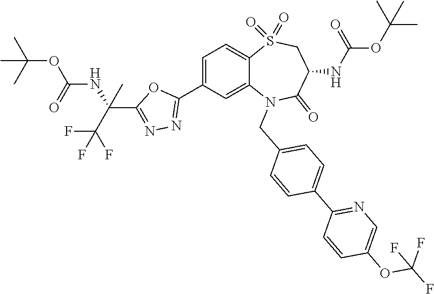 [SMILES: CC(C)(C)OC(=O)N[C@H]1CS(=O)(=O)c2ccc(-c3nnc([C@@](C)(NC(=O)OC(C)(C)C)C(F)(F)F)o3)cc2N(Cc2ccc(-c3ccc(OC(F)(F)F)cn3)cc2)C1=O]

The title compound as prepared from tert-butyl N-[(3R)-7-[5-[(1R)-1-(tert-butoxycarbonylamino)-2,2,2-trifluoro-1-methyl-ethyl]-1,3,4-oxadiazol-2-yl]-1,1,4-trioxo-3,5-dihydro-2H-1λ⁶,5-benzothiazepin-3-yl]carbamate (25 mg, 41.28 μmol, 1.0 eq) and Intermediate 38 (15.1 mg, 45.4 μmol, 1.1 eq) in analogy to general procedure 1a and was obtained as white solid (32 mg, 91%). MS (ESI): 857.5 [M+H]⁺

### Step c) (3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethoxy)-2-pyridyl]phenyl]methyl]-2,3-dihydro-1λ⁶, 5-benzothiazepin-4-one

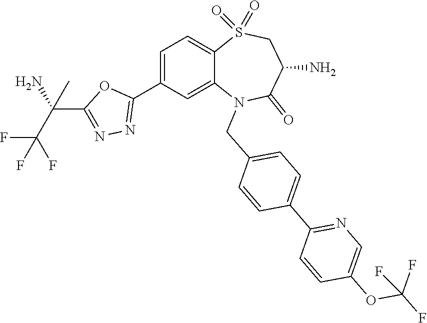 [SMILES: C[C@@](N)(c1nnc(-c2ccc3c(c2)N(Cc2ccc(-c4ccc(OC(F)(F)F)cn4)cc2)C(=O)[C@@H](N)CS3(=O)=O)o1)C(F)(F)F]

The title compound was prepared from tert-butyl N-[(3R)-7-[5-[(1R)-1-(tert-butoxycarbonylamino)-2,2,2-trifluoro-1-methyl-ethyl]-1,3,4-oxadiazol-2-yl]-1,1,4-trioxo-5-[[4-[5-(trifluoromethoxy)-2-pyridyl]phenyl]methyl]-2,3-dihydro-1λ⁶,5-benzothiazepin-3-yl]carbamate (32 mg, 37.35 mol) in analogy to general procedure 11a and was obtained as white solid, as hydrochloride salt (24 mg, 89%). MS (ESI): 657.2 [M+H]⁺

The examples of the following table were prepared in analogy to Example 153, using the appropriate benzyl bromide building block.

| | | | Building | MS, ESI: |
|---|---|---|---|---|
| Ex. | Structure | Systematic Name | Block | m/z |
| 154 | 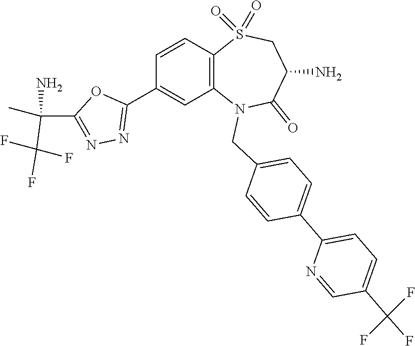 [SMILES: C[C@@](N)(c1nnc(-c2ccc3c(c2)N(Cc2ccc(-c4ccc(C(F)(F)F)cn4)cc2)C(=O)[C@@H](N)CS3(=O)=O)o1)C(F)(F)F] | (3R)-3-amino-7-[5- [(1R)-1-amino-2,2,2- trifluoro-1-methyl- ethyl]-1,3,4-oxadiazol- 2-yl]-1,1-dioxo-5-[[4- [5-(trifluoromethyl)-2- pyridyl]phenyl]methyl]- 2,3-dihydro-1λ⁶,5- benzothiazepin-4-one (*) | 2-[4- (bromomethyl) phenyl]- 5- (trifluoromethyl) pyridine (CAS 1056641- 21-4) | 641.3 [M + H]⁺ |
| 156 | 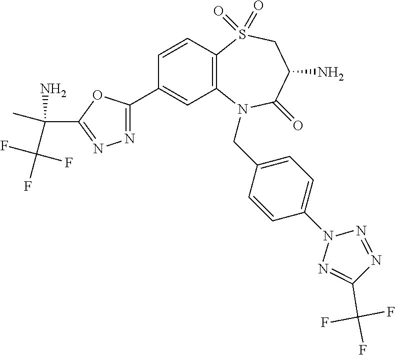 [SMILES: C[C@@](N)(c1nnc(-c2ccc3c(c2)N(Cc2ccc(-n4nnc(C(F)(F)F)n4)cc2)C(=O)[C@@H](N)CS3(=O)=O)o1)C(F)(F)F] | (3R)-3-amino-7-[5- [(1R)-1-amino-2,2,2- trifluoro-1-methyl- ethyl]-1,3,4-oxadiazol- 2-yl]-1,1-dioxo-5-[[4- [5- (trifluoromethyl) tetrazol-2-yl]phenyl] methyl]-2,3-dihydro- 1λ⁶,5-benzothiazepin- 4-one (*) | Intermediate 35 | 632.3 [M + H]⁺ |
| 157 | 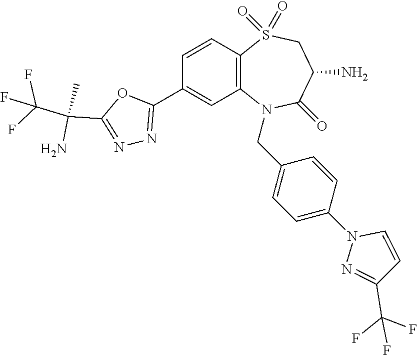 [SMILES: C[C@@](N)(c1nnc(-c2ccc3c(c2)N(Cc2ccc(-n4ccc(C(F)(F)F)n4)cc2)C(=O)[C@@H](N)CS3(=O)=O)o1)C(F)(F)F] | (3R)-3-amino-7-[5- [(1R)-1-amino-2,2,2- trifluoro-1-methyl- ethyl]-1,3,4-oxadiazol- 2-yl]-1,1-dioxo-5-[[4- [3- (trifluoromethyl) pyrazol-1-yl]phenyl] methyl]-2,3-dihydro- 1λ⁶,5-benzothiazepin- 4-one (*) | 1-[4- (Chloromethyl) phenyl]- 3-(trifluoromethyl)- 1H- pyrazole (CAS 1284983- 51-2) | 630.2 [M + H]⁺ |

(*) as hydrochloride salt

### Example 54

### (3R)-3-amino-7-[5-[2-(hydroxymethyl)tetrahydrofuran-2-yl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

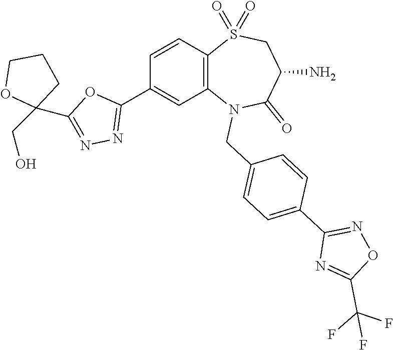 [SMILES: N[C@H]1CS(=O)(=O)c2ccc(-c3nnc(C4(CO)CCCO4)o3)cc2N(Cc2ccc(-c3noc(C(F)(F)F)n3)cc2)C1=O]

### Step a) methyl (3R)-3-(tert-butoxycarbonylamino)-4-oxo-5-[[4-[5-(trifluoromethyl)-1,2,4-oxadiazol-3-yl]phenyl]methyl]-2,3-dihydro-1,5-benzothiazepine-7-carboxylate

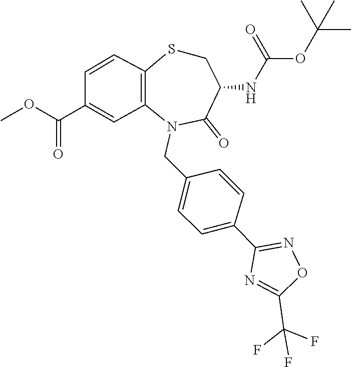 [SMILES: COC(=O)c1ccc2c(c1)N(Cc1ccc(-c3noc(C(F)(F)F)n3)cc1)C(=O)[C@@H](NC(=O)OC(C)(C)C)CS2]

The title compound was prepared from methyl (3R)-3-(tert-butoxycarbonylamino)-4-oxo-3,5-dihydro-2H-1,5-benzothiazepine-7-carboxylate (500 mg, 1.4 mmol, 1.0 eq) and 3-[4-(bromomethyl)phenyl]-5-(trifluoromethyl)-1,2,4-oxadiazole (496 mg, 1.62 mmol, 1.15 eq, CAS 2093101-98-3) in analogy to general procedure 1a and was obtained as white solid (784 mg, 94%). MS (ESI): 523.2 [M-isobutene+H]⁺

### Step b) (3R)-3-(tert-butoxycarbonylamino)-4-oxo-5-[[4-[5-(trifluoromethyl)-1,2,4-oxadiazol-3-yl]phenyl]methyl]-2,3-dihydro-1,5-benzothiazepine-7-carboxylic acid

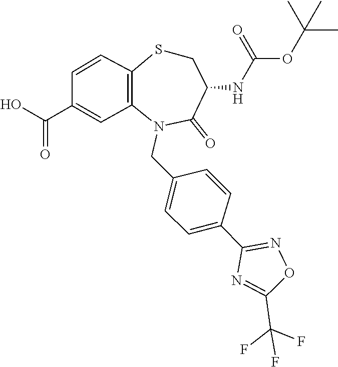 [SMILES: CC(C)(C)OC(=O)N[C@H]1CSc2ccc(C(=O)O)cc2N(Cc2ccc(-c3noc(C(F)(F)F)n3)cc2)C1=O]

The title compound was prepared from methyl (3R)-3-(tert-butoxycarbonylamino)-4-oxo-5-[[4-[5-(trifluoromethyl)-1,2,4-oxadiazol-3-yl]phenyl]methyl]-2,3-dihydro-1,5-benzothiazepine-7-carboxylate (770 mg, 1.33 mmol) in analogy to general procedure 2 and was obtained as light yellow solid (756 mg, 82%). MS (ESI): 509.1 [M-isobutene+H]⁺

### Step c) tert-butyl N-[(3R)-7-(hydrazinecarbonyl)-4-oxo-5-[[4-[5-(trifluoromethyl)-1,2,4-oxadiazol-3-yl]phenyl]methyl]-2,3-dihydro-1,5-benzothiazepin-3-yl]carbamate

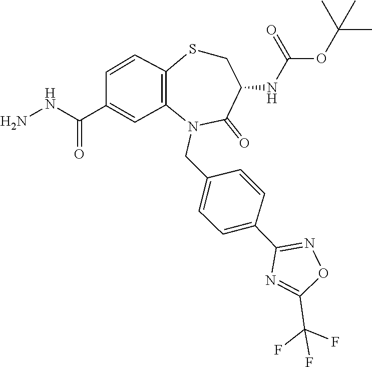 [SMILES: CC(C)(C)OC(=O)N[C@H]1CSc2ccc(C(=O)NN)cc2N(Cc2ccc(-c3noc(C(F)(F)F)n3)cc2)C1=O]

The title compound was prepared from (3R)-3-(tert-butoxycarbonylamino)-4-oxo-5-[[4-[5-(trifluoromethyl)-1,2,4-oxadiazol-3-yl]phenyl]methyl]-2,3-dihydro-1,5-benzothiazepine-7-carboxylic acid (750 mg, 1.08 mmol) in analogy to general procedure 3 and was obtained as white solid (552 mg, 86%). MS (ESI): 523.1 [M-isobutene+H]⁺

### Step d) tert-butyl N-[(3R)-7-[[[2-(hydroxymethyl)tetrahydrofuran-2-carbonyl]amino]carbamoyl]-4-oxo-5-[[4-[5-(trifluoromethyl)-1,2,4-oxadiazol-3-yl]phenyl]methyl]-2,3-dihydro-1,5-benzothiazepin-3-yl]carbamate

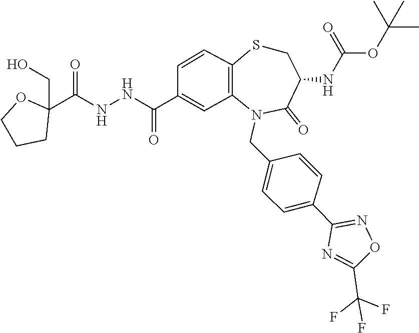 [SMILES: CC(C)(C)OC(=O)N[C@H]1CSc2ccc(C(=O)NNC(=O)C3(CO)CCCO3)cc2N(Cc2ccc(-c3noc(C(F)(F)F)n3)cc2)C1=O]

The title compound was prepared from tert-butyl N-[(3R)-7-(hydrazinecarbonyl)-4-oxo-5-[[4-[5-(trifluoromethyl)-1,2,4-oxadiazol-3-yl]phenyl]methyl]-2,3-dihydro-1,5-benzothiazepin-3-yl]carbamate (100 mg, 0.168 mmol, 1.0 eq) and 2-(hydroxymethyl)tetrahydrofruna-2-carboxylic acid (49 mg, 0.335 mmol, 2.0 eq, CAS 442877-01-2) in analogy to general procedure 4a and was obtained as white solid (97 mg, 60%). MS (ESI): 607.1 [M−Boc+H]⁺

### Step e) tert-butyl N-[(3R)-7-[[[2-[[tert-butyl(dimethyl)silyl]oxymethyl]tetrahydrofuran-2-carbonyl]amino]carbamoyl]-4-oxo-5-[[4-[5-(trifluoromethyl)-1,2,4-oxadiazol-3-yl]phenyl]methyl]-2,3-dihydro-1,5-benzothiazepin-3-yl]carbamate

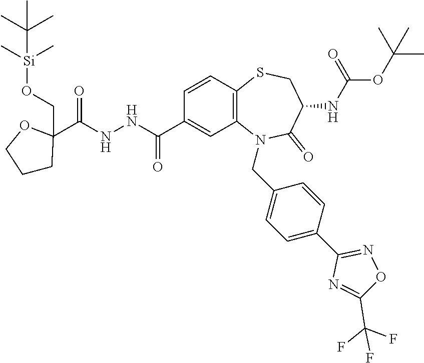 [SMILES: CC(C)(C)OC(=O)N[C@H]1CSc2ccc(C(=O)NNC(=O)C3(CO[Si](C)(C)C(C)(C)C)CCCO3)cc2N(Cc2ccc(-c3noc(C(F)(F)F)n3)cc2)C1=O]

To a solution of tert-butyl N-[(3R)-7-[[[2-(hydroxymethyl)tetrahydrofuran-2-carbonyl]amino]carbamoyl]-4-oxo-5-[[4-[5-(trifluoromethyl)-1,2,4-oxadiazol-3-yl]phenyl]methyl]-2,3-dihydro-1,5-benzothiazepin-3-yl]carbamate (97 mg, 0.1 mmol, 1.0 eq) in DCM (1 ml) was added TBDMS-Cl (22.65 mg, 0.15 mmol, 1.5 eq) and imidazole (17.05 mg, 0.25 mmol, 2.5 eq) at RT overnight. The reaction mixture was directly concentrated and purified by flash column chromatography on silica gel (0-60% EtOAc in heptane) to afford the title compound as white solid (66 mg, 80%). MS (ESI): 765.3 [M-isobutene+H]⁺

### Step f) tert-butyl N-[(3R)-7-[5-[2-[[tert-butyl(dimethyl)silyl]oxymethyl]tetrahydrofuran-2-yl]-1,3,4-oxadiazol-2-yl]-4-oxo-5-[[4-[5-(trifluoromethyl)-1,2,4-oxadiazol-3-yl]phenyl]methyl]-2,3-dihydro-1,5-benzothiazepin-3-ylcarbamate

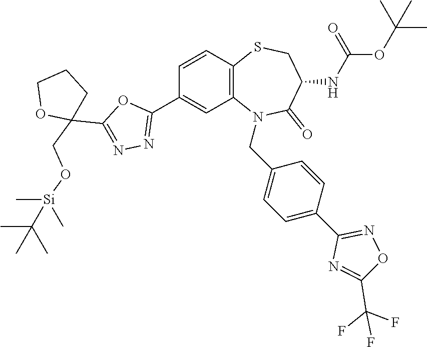 [SMILES: CC(C)(C)OC(=O)N[C@H]1CSc2ccc(-c3nnc(C4(CO[Si](C)(C)C(C)(C)C)CCCO4)o3)cc2N(Cc2ccc(-c3noc(C(F)(F)F)n3)cc2)C1=O]

The title compound was prepared from tert-butyl N-[(3R)-7-[[[2-[[tert-butyl(dimethyl)silyl]oxymethyl]tetrahydrofuran-2-carbonyl]amino]carbamoyl]-4-oxo-5-[[4-[5-(trifluoromethyl)-1,2,4-oxadiazol-3-yl]phenyl]methyl]-2,3-dihydro-1,5-benzothiazepin-3-yl]carbamate (66 mg, 0.08 mmol) in analogy to general procedure 5b and was obtained as colorless solid (41 mg, 61%). MS (ESI): 803.5 [M+H]⁺

### Step g) tert-butyl N-[(3R)-7-[5-[2-[[tert-butyl(dimethyl)silyl]oxymethyl]tetrahydrofuran-2-yl]-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶, 5-benzothiazepin-3-yl]carbamate

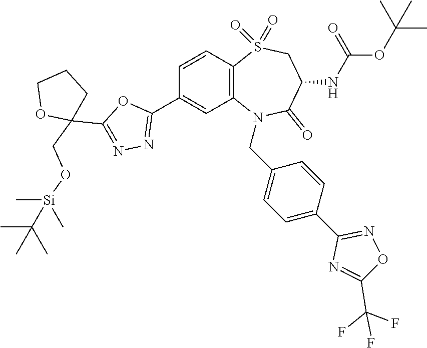 [SMILES: CC(C)(C)OC(=O)N[C@H]1CS(=O)(=O)c2ccc(-c3nnc(C4(CO[Si](C)(C)C(C)(C)C)CCCO4)o3)cc2N(Cc2ccc(-c3noc(C(F)(F)F)n3)cc2)C1=O]

The title compound was prepared from tert-butyl N-[(3R)-7-[5-[2-[[tert-butyl(dimethyl)silyl]oxymethyl]tetrahydrofuran-2-yl]-1,3,4-oxadiazol-2-yl]-4-oxo-5-[[4-[5-(trifluoromethyl)-1,2,4-oxadiazol-3-yl]phenyl]methyl]-2,3-dihydro-1,5-benzothiazepin-3-yl]carbamate (41 mg, 0.049 mmol) in analogy to general procedure 10 and was obtained as white solid (40 mg, 98%). MS (ESI): 835.3 [M+H]⁺

### Step h) (3R)-3-amino-7-[5-[2-(hydroxymethyl)tetrahydrofuran-2-yl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one

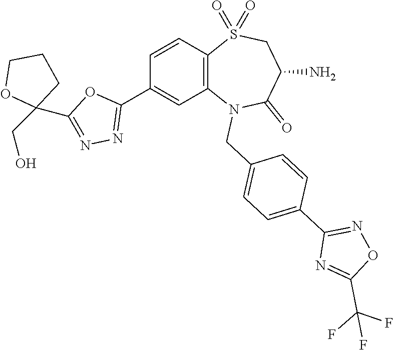 [SMILES: N[C@H]1CS(=O)(=O)c2ccc(-c3nnc(C4(CO)CCCO4)o3)cc2N(Cc2ccc(-c3noc(C(F)(F)F)n3)cc2)C1=O]

The title compound was prepared from tert-butyl N-[(3R)-7-[5-[2-[[tert-butyl(dimethyl)silyl]oxymethyl]tetrahydrofuran-2-yl]-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (40 mg, 0.048 mmol) in analog to general procedure 11a and was obtained as white solid, as hydrochloride salt (18 mg, 57%). MS (ESI): 621.1 [M+H]⁺

The examples of the following table were prepared in analogy to Example 54, using the appropriate benzyl bromide building block.

| | | | | MS, |
|---|---|---|---|---|
| | | | Building | ESI: |
| Ex. | Structure | Systematic Name | Block | m/z |
| 55 | 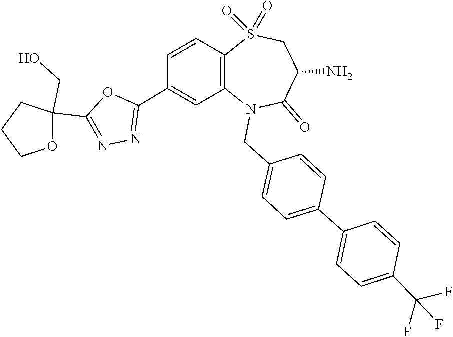 [SMILES: N[C@H]1CS(=O)(=O)c2ccc(-c3nnc(C4(CO)CCCO4)o3)cc2N(Cc2ccc(-c3ccc(C(F)(F)F)cc3)cc2)C1=O] | (3R)-3-amino-7- [5-[2- (hydroxymethyl) tetrahydrofuran-2- yl]-1,3,4-oxadiazol- 2-yl]-1,1-dioxo-5- [[4-[4- (trifluoromethyl) phenyl] phenyl]methyl]-2,3- dihydro-1λ⁶,5- benzothiazepin-4- one | 4- (bromomethyl)- 4′- (trifluoromethyl)- 1,1′- biphenyl (CAS 613241-14-8) | 629.3 [M + H]⁺ |

### Example 56

### methyl 3,3-difluoro-5-[5-[(3R)-3-amino-5-[[4-(5-tert-butyl-1,2,4-oxadiazol-3-yl)phenyl]methyl]-1,1,4-trioxo-2,3-dihydro-1λ⁶,5-benzothiazepin-7-yl]-1,3,4-oxadiazol-2-yl]piperidine-1-carboxylate

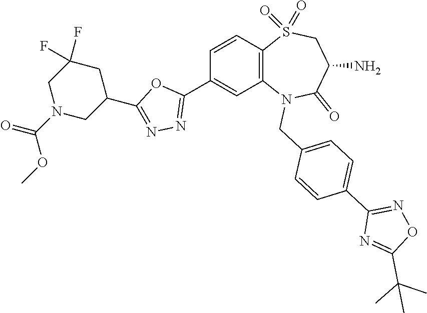 [SMILES: COC(=O)N1CC(c2nnc(-c3ccc4c(c3)N(Cc3ccc(-c5noc(C(C)(C)C)n5)cc3)C(=O)[C@@H](N)CS4(=O)=O)o2)CC(F)(F)C1]

### Step a) benzyl 3,3-difluoro-5-[[[(3R)-3-(tert-butoxycarbonylamino)-4-oxo-3,5-dihydro-2H-1λ⁶,5-benzothiazepine-7-carbonyl]amino]carbamoyl]piperidine-1-carboxylate

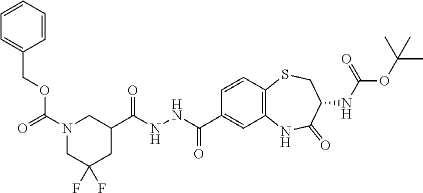 [SMILES: CC(C)(C)OC(=O)N[C@H]1CSc2ccc(C(=O)NNC(=O)C3CN(C(=O)OCc4ccccc4)CC(F)(F)C3)cc2NC1=O]

The title compound was prepared from tert-butyl N-[(3R)-7-(hydrazinecarbonyl)-4-oxo-3,5-dihydro-2H-1,5-benzothiazepin-3-yl]carbamate (980 mg, 3.27 mmol, Example 19, step b) and 5,5-difluoro-1-[(E)-2-vinylbut-2-enoxy]carbonyl-piperidine-3-carboxylic acid (980 mg, 3.3 mmol, 1,2 eq. CAS 1356338-81-2) in analogy to general procedure 4b and was obtained as light-yellow solid (1.08 g, 63% yield. MS (ESI): 534.1 [M+H-isobutene]⁺

### Step b) benzyl 3,3-difluoro-5-[5-[(3R)-3-(tert-butoxycarbonylamino)-4-oxo-3,5-dihydro-2H-1λ⁶,5-benzothiazepin-7-yl]-1,3,4-oxadiazol-2-yl]piperidine-1-carboxylate

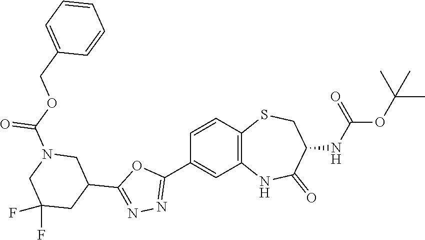 [SMILES: CC(C)(C)OC(=O)N[C@H]1CSc2ccc(-c3nnc(C4CN(C(=O)OCc5ccccc5)CC(F)(F)C4)o3)cc2NC1=O]

The title compound was prepared from benzyl 3,3-difluoro-5-[[[(3R)-3-(tert-butoxycarbonylamino)-4-oxo-3,5-dihydro-2H-1λ⁶,5-benzothiazepine-7-carbonyl]amino]carbamoyl]piperidine-1-carboxylate (1060 mg, 1.67 mmol) in analogy to general procedure 5a and was obtained as light yellow foam (721 mg, 70% yield). MS (ESI): 560.1 [M+H-isobutene]⁺

### Step c) benzyl 3,3-difluoro-5-[5-[(3R)-3-(tert-butoxycarbonylamino)-5-[[4-(5-tert-butyl-1,2,4-oxadiazol-3-yl)phenyl]methyl]-4-oxo-2,3-dihydro-1,5-benzothiazepin-7yl]-1,3,4-oxadiazol-2-yl]piperidine-]-carboxylate

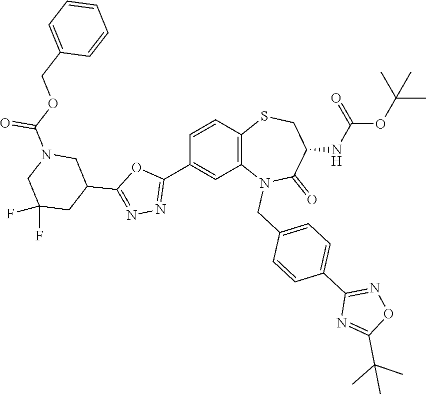 [SMILES: CC(C)(C)OC(=O)N[C@H]1CSc2ccc(-c3nnc(C4CN(C(=O)OCc5ccccc5)CC(F)(F)C4)o3)cc2N(Cc2ccc(-c3noc(C(C)(C)C)n3)cc2)C1=O]

The title compound was prepared from benzyl 3,3-difluoro-5-[5-[(3R)-3-(tert-butoxycarbonylamino)-4-oxo-3,5-dihydro-2H-1λ⁶,5-benzothiazepin-7-yl]-1,3,4-oxadiazol-2-yl]piperidine-1-carboxylate (615 mg, 0.6 mmol, 1.0 eq,) and Intermediate 13 (184.6 mg, 0.6 mmol, 1,1 eq.) in analogy to general procedure 1a and was obtained as light yellow foam (721 mg, 70% yield). MS (ESI): 830.3 [M+H]⁺

### Step d) benzyl 3,3-difluoro-5-[5-[(3R)-3-(tert-butoxycarbonylamino)-5-[[4-(5-tert-butyl-1,2,4-oxadiazol-3-yl)phenyl]methyl]-1,1,4-trioxo-2,3-dihydro-1λ⁶,5-benzothiazepin-7-yl]-1,3,4-oxadiazol-2-yl]piperidine-1-carboxylate

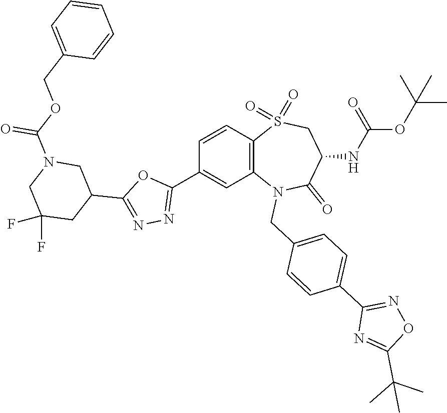 [SMILES: CC(C)(C)OC(=O)N[C@H]1CS(=O)(=O)c2ccc(-c3nnc(C4CN(C(=O)OCc5ccccc5)CC(F)(F)C4)o3)cc2N(Cc2ccc(-c3noc(C(C)(C)C)n3)cc2)C1=O]

The title compound was prepared from benzyl 3,3-difluoro-5-[5-[(3R)-3-(tert-butoxycarbonylamino)-5-[[4-(5-tert-butyl-1,2,4-oxadiazol-3-yl)phenyl]methyl]-4-oxo-2,3-dihydro-1,5-benzothiazepin-7-yl]-1,3,4-oxadiazol-2-yl]piperidine-1-carboxylate (363 mg, 0.4 mmol) in analogy to general procedure 10 and was obtained as light yellow solid (374 mg, 0.43 mmol, 99% yield). MS (ESI): 762.2 [M+H-isobutene]⁺

### Step e) tert-butyl N-[(3R)-5-[[4-(5-tert-butyl-1,2,4-oxadiazol-3-yl)phenyl]methyl]-7-[5-(5,5-difluoro-3-piperidyl)-1,3,4-oxadiazol-2-yl]-1,1,4-trioxo-2,3-dihydro-1λ⁶, 5-benzothiazepin-3-yl]carbamate

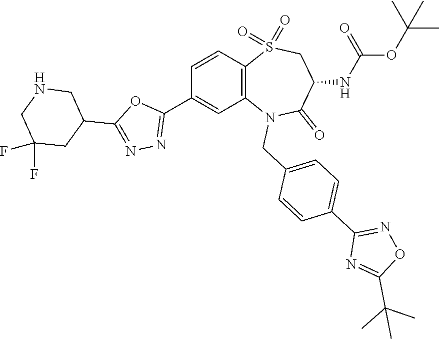 [SMILES: CC(C)(C)OC(=O)N[C@H]1CS(=O)(=O)c2ccc(-c3nnc(C4CNCC(F)(F)C4)o3)cc2N(Cc2ccc(-c3noc(C(C)(C)C)n3)cc2)C1=O]

To a solution of benzyl 3,3-difluoro-5-[5-[(3R)-3-(tert-butoxycarbonylamino)-5-[[4-(5-tert-butyl-1,2,4-oxadiazol-3-yl)phenyl]methyl]-1,1,4-trioxo-2,3-dihydro-1λ⁶,5-benzothiazepin-7-yl]-1,3,4-oxadiazol-2-yl]piperidine-1-carboxylate (324 mg, 0.4 mmol, 1.0 eq) in MeOH (10 mL) under inert atmosphere. The reaction mixture was stirred under hydrogen atmosphere for 5 hours. The mixture was filtered through a plug of celite, washing with MeOH. The filtrate was concentrated to afford tert-butyl N-[(3R)-5-[[4-(5-tert-butyl-1,2,4-oxadiazol-3-yl)phenyl]methyl]-7-[5-(5,5-difluoro-3-piperidyl)-1,3,4-oxadiazol-2-yl]-1,1,4-trioxo-2,3-dihydro-1λ⁶,5-benzothiazepin-3-yl]carbamate (244 mg, 0.34 mmol, 89% yield) as yellow foam. MS (ESI): 728.2 [M+H]⁺

### Step f) methyl 3,3-difluoro-5-[5-[(3R)-3-(tert-butoxycarbonylamino)-5-[[4-(5-tert-butyl-1,2,4-oxadiazol-3-yl)phenyl]methyl]-1,1,4-trioxo-2,3-dihydro-1λ⁶, 5-benzothiazepin-7yl]-1,3,4-oxadiazol-2-yl]piperidine-1-carboxylate

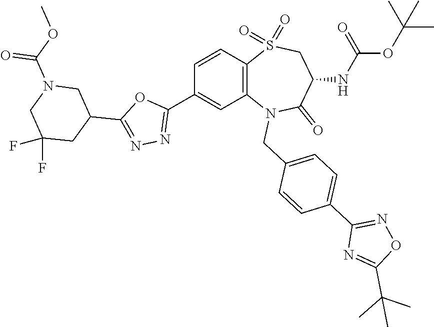 [SMILES: COC(=O)N1CC(c2nnc(-c3ccc4c(c3)N(Cc3ccc(-c5noc(C(C)(C)C)n5)cc3)C(=O)[C@@H](NC(=O)OC(C)(C)C)CS4(=O)=O)o2)CC(F)(F)C1]

To a solution of tert-butyl N-[(3R)-5-[[4-(5-tert-butyl-1,2,4-oxadiazol-3-yl)phenyl]methyl]-7-[5-(5,5-difluoro-3-piperidyl)-1,3,4-oxadiazol-2-yl]-1,1,4-trioxo-2,3-dihydro-1λ⁶,5-benzothiazepin-3-yl]carbamate (122 mg, 0.2 mmol, 1.0 eq) and N,N-diisopropylethylamine (0.0876 mL, 0.5 mmol, 3.0 eq) in DCM (3.0 mL) was added methoxycarbonyl methyl carbonate (0.02 mL, 0.17 mmol, 1.0 eq. CAS 4525-33-1) at 0° C. The mixture was stirred for 30 min at RT. The reaction mixture was concentrated in vacuo to afford crude product. The crude product was purified by prep-HPLC (Neutral), the eluent was concentrated in vacuo to remove part of acetonitrile, then dried by lyophilization to afford the desired title compound (70 mg, 0.09 mmol, 53% yield) as white solid. MS (ESI): 786.2 [M+H]⁺

### Step g) methyl 3,3-difluoro-5-[5-[(3R)-3-amino-5-[[4-(5-tert-butyl-1,2,4-oxadiazol-3-yl)phenyl]methyl]-1,1,4-trioxo-2,3-dihydro-1λ⁶,5-benzothiazepin-7-yl]-1,3,4-oxadiazol-2-yl]piperidine-1-carboxylate

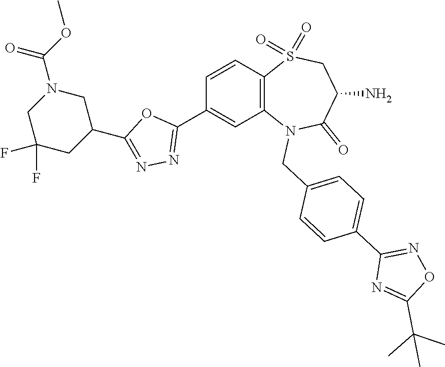 [SMILES: COC(=O)N1CC(c2nnc(-c3ccc4c(c3)N(Cc3ccc(-c5noc(C(C)(C)C)n5)cc3)C(=O)[C@@H](N)CS4(=O)=O)o2)CC(F)(F)C1]

The title compound was prepared from methyl 3,3-difluoro-5-[5-[(3R)-3-(tert-butoxycarbonylamino)-5-[[4-(5-tert-butyl-1,2,4-oxadiazol-3-yl)phenyl]methyl]-1,1,4-trioxo-2,3-dihydro-1λ⁶,5-benzothiazepin-7-yl]-1,3,4-oxadiazol-2-yl]piperidine-1-carboxylate (60 mg, 0.1 mmol) in analogy to general procedure 11c and was obtained as a white solid, as hydrochloride salt (42.3 mg, 0.06 mmol, 75% yield). MS (ESI): 686.3 [M+H]⁺

### Example 57

### methyl 3,3-difluoro-5-[5-[(3R)-3-amino-1,1,4-trioxo-5-[[6-[4-(trifluoromethyl)phenyl]-3-pyridyl]methyl]-2,3-dihydro-1λ⁶,5-benzothiazepin-7-yl]-1,3,4-oxadiazol-2-yl]piperidine-1-carboxylate

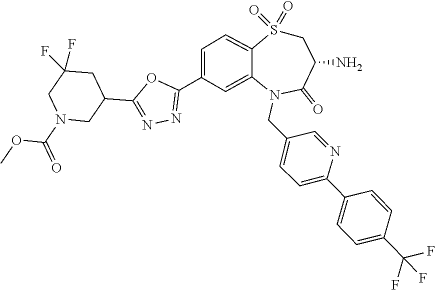 [SMILES: COC(=O)N1CC(c2nnc(-c3ccc4c(c3)N(Cc3ccc(-c5ccc(C(F)(F)F)cc5)nc3)C(=O)[C@@H](N)CS4(=O)=O)o2)CC(F)(F)C1]

### Step a) benzyl 3,3-difluoro-5-[[[(3R)-3-(tert-butoxycarbonylamino)-4-oxo-3,5-dihydro-2H-1,5-benzothiazepine-7-carbonyl]amino]carbamoyl]piperidine-1-carboxylate

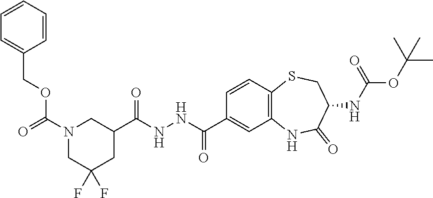 [SMILES: CC(C)(C)OC(=O)N[C@H]1CSc2ccc(C(=O)NNC(=O)C3CN(C(=O)OCc4ccccc4)CC(F)(F)C3)cc2NC1=O]

The title compound was prepared from tert-butyl N-[(3R)-7-(hydrazinecarbonyl)-4-oxo-3,5-dihydro-2H-1,5-benzothiazepin-3-yl]carbamate (980 mg, 3.27 mmol, Example 19, step b) and 5,5-difluoro-1-[(E)-2-vinylbut-2-enoxy]carbonyl-piperidine-3-carboxylic acid (980 mg, 3.3 mmol, 1,2 eq. CAS 1356338-81-2) in analogy to general procedure 4b and was obtained as light yellow foam (1.08 g, 63% yield) MS (ESI): 534.1 [M+H-isobutene]⁺

### Step b) benzyl 3,3-difluoro-5-[5-[(3R)-3-(tert-butoxycarbonylamino)-4-oxo-3,5-dihydro-2H-1λ⁶,5-benzothiazepin-7-yl]-1,3,4-oxadiazol-2-yl]piperidine-1-carboxylate

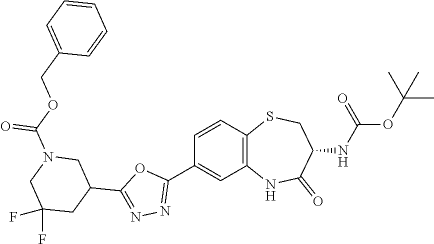 [SMILES: CC(C)(C)OC(=O)N[C@H]1CSc2ccc(-c3nnc(C4CN(C(=O)OCc5ccccc5)CC(F)(F)C4)o3)cc2NC1=O]

The title compound was prepared from benzyl 3,3-difluoro-5-[[[(3R)-3-(tert-butoxycarbonylamino)-4-oxo-3,5-dihydro-2H-1λ⁶,5-benzothiazepine-7-carbonyl]amino]carbamoyl]piperidine-1-carboxylate (1060 mg, 1.67 mmol) in analogy to general procedure 5a and was obtained as light yellow foam (721 mg, 70% yield). MS (ESI): 560.1 [M+H-isobutene]⁺

### Step c) benzyl 3,3-difluoro-5-[5-[(3R)-3-(tert-butoxycarbonylamino)-1,1,4-trioxo-3,5-dihydro-2H-1λ⁶,5-benzothiazepin-7-yl]-1,3,4-oxadiazol-2-yl]piperidine-1-carboxylate

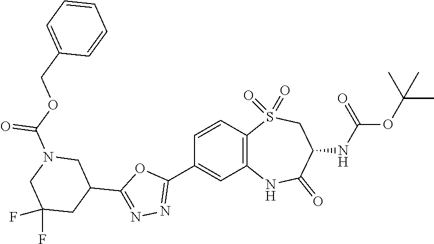 [SMILES: CC(C)(C)OC(=O)N[C@H]1CS(=O)(=O)c2ccc(-c3nnc(C4CN(C(=O)OCc5ccccc5)CC(F)(F)C4)o3)cc2NC1=O]

The title compound was prepared from benzyl 3,3-difluoro-5-[5-[(3R)-3-(tert-butoxycarbonylamino)-4-oxo-3,5-dihydro-2H-1λ⁶,5-benzothiazepin-7-yl]-1,3,4-oxadiazol-2-yl]piperidine-1-carboxylate (300 mg, 0.49 mmol, 1.0 eq) in analogy to general procedure 10 and was obtained as light yellow solid (320 mg, 0.49 mmol, 92% yield). MS (ESI): 592.1 [M+H-isobutene]⁺

### Step d) benzyl 3,3-difluoro-5-[5-[(3R)-3-(tert-butoxycarbonylamino)-1,1,4-trioxo-5-[[6-[4-(trifluoromethyl)phenyl]-3-pyridyl]methyl]-2,3-dihydro-1λ⁶,5-benzothiazepin-7yl]-1,3,4-oxadiazol-2-yl]piperidine-1-carboxylate

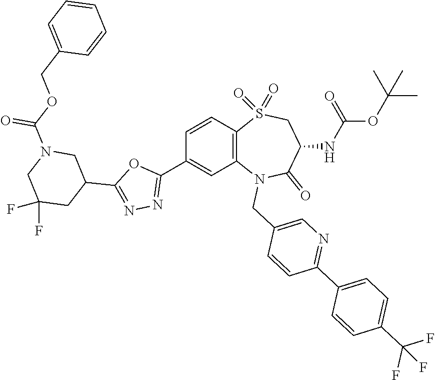 [SMILES: CC(C)(C)OC(=O)N[C@H]1CS(=O)(=O)c2ccc(-c3nnc(C4CN(C(=O)OCc5ccccc5)CC(F)(F)C4)o3)cc2N(Cc2ccc(-c3ccc(C(F)(F)F)cc3)nc2)C1=O]

The title compound was prepared from benzyl 3,3-difluoro-5-[5-[(3R)-3-(tert-butoxycarbonylamino)-1,1,4-trioxo-3,5-dihydro-2H-1λ⁶,5-benzothiazepin-7-yl]-1,3,4-oxadiazol-2-yl]piperidine-1-carboxylate (300 mg, 0.46 mmol, 1,0 eq) and 5-(Chloromethyl)-2-[4-(trifluoromethyl)phenyl]pyridine hydrochloride (185.6 mg, 0.6 mmol, 1.3 eq. CAS 851507-54-5) in analogy to general procedure 1a and was obtained as light yellow oil (570 mg, 106% yield). MS (ESI): 883.2 [M+H]⁺

### Step e) tert-butyl N-[(3R)-7-[5-(5,5-difluoro-3-piperidyl)-1,3,4-oxadiazol-2-yl]-1,1,4-trioxo-5-[[6-[4-(trifluoromethyl)phenyl]-3-pyridyl]methyl]-2,3-dihydro-1λ⁶,5-benzothiazepin-3-yl]carbamate

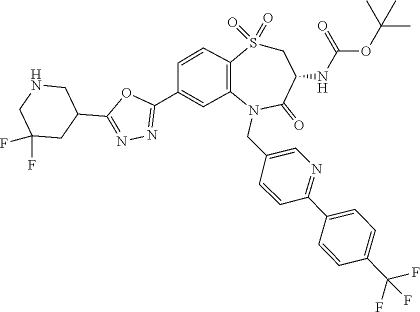 [SMILES: CC(C)(C)OC(=O)N[C@H]1CS(=O)(=O)c2ccc(-c3nnc(C4CNCC(F)(F)C4)o3)cc2N(Cc2ccc(-c3ccc(C(F)(F)F)cc3)nc2)C1=O]

To a solution of benzyl 3,3-difluoro-5-[5-[(3R)-3-(tert-butoxycarbonylamino)-1,1,4-trioxo-5-[[6-[4-(trifluoromethyl)phenyl]-3-pyridyl]methyl]-2,3-dihydro-1λ⁶,5-benzothiazepin-7-yl]-1,3,4-oxadiazol-2-yl]piperidine-1-carboxylate (50 mg, 0.06 mmol, 1.0 eq) in MeOH (5 mL) was added Pd/C (50 mg) under inert atmosphere. The reaction mixture was stirred under hydrogen atmosphere for 5 hours. The mixture was filtered through a plug of celite, washing with MeOH. The filtrate was concentrated to afford the title compound as a light yellow oil (30 mg, 0.04 mmol, 47% yield). MS (ESI): 749.3 [M+H]⁺

### Step f) methyl 3,3-difluoro-5-[5-[(3R)-3-(tert-butoxycarbonylamino)-1,1,4-trioxo-5-[[6-[4-(trifluoromethyl)phenyl]-3-pyridyl]methyl]-2,3-dihydro-1λ⁶,5-benzothiazepin-7-yl]-1,3,4-oxadiazol-2-yl]piperidine-1-carboxylate

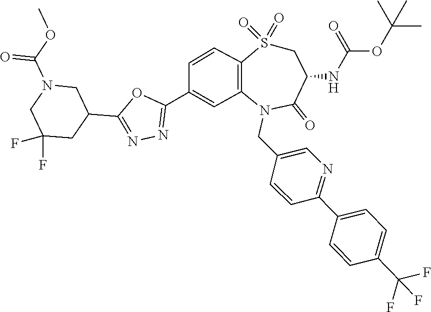 [SMILES: COC(=O)N1CC(c2nnc(-c3ccc4c(c3)N(Cc3ccc(-c5ccc(C(F)(F)F)cc5)nc3)C(=O)[C@@H](NC(=O)OC(C)(C)C)CS4(=O)=O)o2)CC(F)(F)C1]

To a solution of tert-butyl N-[(3R)-7-[5-(5,5-difluoro-3-piperidyl)-1,3,4-oxadiazol-2-yl]-1,1,4-trioxo-5-[[6-[4-(trifluoromethyl)phenyl]-3-pyridyl]methyl]-2,3-dihydro-1λ⁶,5-benzothiazepin-3-yl]carbamate (45 mg, 0.06 mmol, 1.0 eq) and N,N-diisopropylethylamine (0.03 mL, 0.18 mmol, 3.0 eq.) in DCM (2.0 mL) was added methoxycarbonyl methyl carbonate (16.12 mg, 0.17 mmol, 1.0 eq. CAS 4525-33-1) at 0° C. for 30 min. After complete addition, the mixture was stirred for 12 hour at RT. The reaction mixture was poured into water (10 mL) and the layers were separated. The aqueous phase was extracted with EtOAc (3×10 mL). The combined extracts were washed with brine (10 mL), dried over anhydrous sodium sulfate, filtered, and concentrated under reduced pressure. The remaining crude product was purified by prep-HPLC (Neutural). After drying by lyophilization, the title compound was obtained as light yellow oil (30 mg, 0.04 mmol, 56% yield). MS (ESI): 807.3 [M+H]⁺

### Step g) methyl 3,3-difluoro-5-[5-[(3R)-3-amino-1,1,4-trioxo-5-[[6-[4-(trifluoromethyl)phenyl]-3-pyridyl]methyl]-2,3-dihydro-1λ⁶, 5-benzothiazepin-7-yl]-1,3,4-oxadiazol-2-yl]piperidine-1-carboxylate

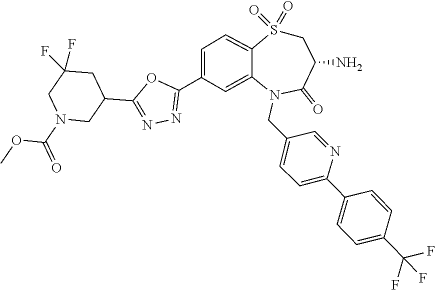 [SMILES: COC(=O)N1CC(c2nnc(-c3ccc4c(c3)N(Cc3ccc(-c5ccc(C(F)(F)F)cc5)nc3)C(=O)[C@@H](N)CS4(=O)=O)o2)CC(F)(F)C1]

The title compound was prepared from methyl 3,3-difluoro-5-[5-[(3R)-3-(tert-butoxycarbonylamino)-1,1,4-trioxo-5-[[6-[4-(trifluoromethyl)phenyl]-3-pyridyl]methyl]-2,3-dihydro-1λ⁶,5-benzothiazepin-7-yl]-1,3,4-oxadiazol-2-yl]piperidine-1-carboxylate (14 mg, 0.02 mmol) in analogy to general procedure 11c and was obtained as a white solid, as hydrochloride salt (9.2 mg, 0.01 mmol, 70% yield). MS (ESI): 707.2 [M+H]⁺

### Example 58

### methyl 1-[5-[(3R)-3-amino-1,1,4-trioxo-5-[[6-[4-(trifluoromethyl)phenyl]-3-pyridyl]methyl]-2,3-dihydro-1λ⁶,5-benzothiazepin-7-yl]-1,3,4-oxadiazol-2-yl]-3-azabicyclo[3.1.1]heptane-3-carboxylate

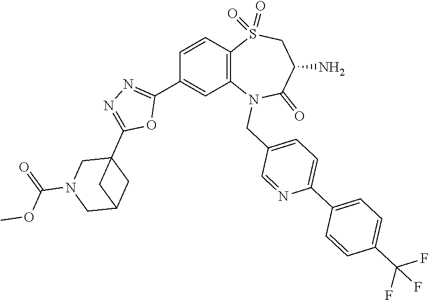 [SMILES: COC(=O)N1CC2CC(c3nnc(-c4ccc5c(c4)N(Cc4ccc(-c6ccc(C(F)(F)F)cc6)nc4)C(=O)[C@@H](N)CS5(=O)=O)o3)(C2)C1]

### Step a) methyl (3R)-3-(tert-butoxycarbonylamino)-1,1,4-trioxo-3,5-dihydro-2H-1λ⁶,5-benzothiazepine-7-carboxylate

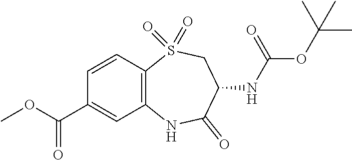 [SMILES: COC(=O)c1ccc2c(c1)NC(=O)[C@@H](NC(=O)OC(C)(C)C)CS2(=O)=O]

The title compound was prepared from methyl (3R)-3-(tert-butoxycarbonylamino)-4-oxo-3,5-dihydro-2H-1,5-benzothiazepine-7-carboxylate (1000 mg, 2.84 mmol, CAS 2089150-62-7) in analogy to general procedure 10 and was obtained as light yellow solid (900 mg, 2.34 mmol, 78% yield). MS (ESI): 285.1 [M+H-Boc]⁺

### Step b) methyl (3R)-3-(tert-butoxycarbonylamino)-1,1,4-trioxo-5-[[6-[4-(trifluoromethyl)phenyl]-3-pyridyl]methyl]-2,3-dihydro-1λ⁶, 5-benzothiazepine-7-carboxylate

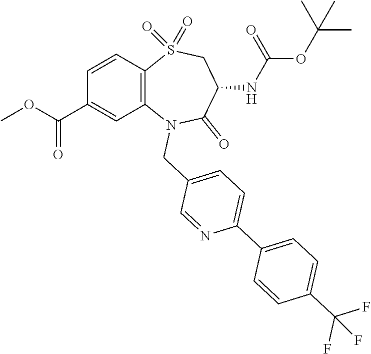 [SMILES: COC(=O)c1ccc2c(c1)N(Cc1ccc(-c3ccc(C(F)(F)F)cc3)nc1)C(=O)[C@@H](NC(=O)OC(C)(C)C)CS2(=O)=O]

The title compound was prepared from methyl (3R)-3-(tert-butoxycarbonylamino)-1,1,4-trioxo-3,5-dihydro-2H-1λ⁶,5-benzothiazepine-7-carboxylate (250 mg, 0.65 mmol) and 5-(Chloromethyl)-2-[4-(trifluoromethyl)phenyl]pyridine (260.5 mg, 0.85 mmol, 1.3 eq. CAS 851507-54-5) in analogy to general procedure 1a and was obtained as white solid (480 mg, 0.77 mmol, 83% yield). MS (ESI): 620.3 [M+H]⁺.

### Step c) (3R)-3-(tert-butoxycarbonylamino)-1,1,4-trioxo-5-[[6-[4-(trifluoromethyl)phenyl]-3-pyridyl]methyl]-2,3-dihydro-1λ⁶,5-benzothiazepine-7-carboxylic acid

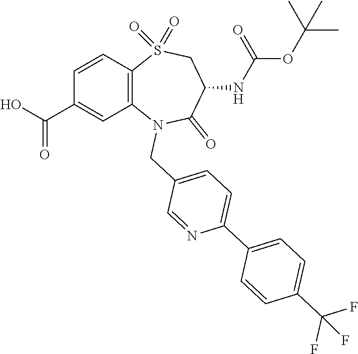 [SMILES: CC(C)(C)OC(=O)N[C@H]1CS(=O)(=O)c2ccc(C(=O)O)cc2N(Cc2ccc(-c3ccc(C(F)(F)F)cc3)nc2)C1=O]

The title compound was prepared from methyl (3R)-3-(tert-butoxycarbonylamino)-1,1,4-trioxo-5-[[6-[4-(trifluoromethyl)phenyl]-3-pyridyl]methyl]-2,3-dihydro-1λ⁶,5-benzothiazepine-7-carboxylate (430 mg, 0.69 mmol) in analogy to general procedure 2 and was obtained as light yellow solid (450 mg, 0.74 mmol, 94% yield). MS (ESI): 606.3 [M+H]⁺.

### Step d) tert-butyl N-[(3R)-7-(hydrazinecarbonyl)-1,1,4-trioxo-5-[[6-[4-(trifluoromethyl)phenyl]-3-pyridyl]methyl]-2,3-dihydro-1λ⁶, 5-benzothiazepin-3-yl]carbamate

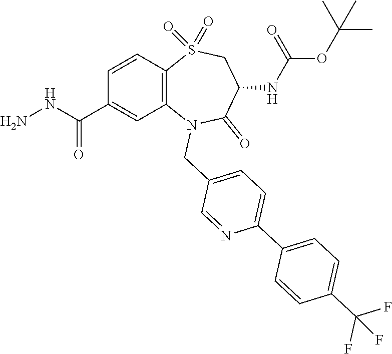 [SMILES: CC(C)(C)OC(=O)N[C@H]1CS(=O)(=O)c2ccc(C(=O)NN)cc2N(Cc2ccc(-c3ccc(C(F)(F)F)cc3)nc2)C1=O]

The title compound was prepared from (3R)-3-(tert-butoxycarbonylamino)-1,1,4-trioxo-5-[[6-[4-(trifluoromethyl)phenyl]-3-pyridyl]methyl]-2,3-dihydro-1λ⁶,5-benzothiazepine-7-carboxylic acid (560 mg, 0.9 mmol) in analogy to general procedure 3 and was obtained as light yellow solid (310 mg, 0.5 mmol, 54% yield). MS (ESI): 620.3 [M+H]⁺.

### Step e) tert-butyl 1-[[[(3R)-3-(tert-butoxycarbonylamino)-1,1,4-trioxo-5-[[6-[4-(trifluoromethyl)phenyl]-3-pyridyl]methyl]-2,3-dihydro-1λ⁶,5-benzothiazepine-7-carbonyl]amino]carbamoyl]-3-azabicyclo[3.1.1]heptane-3-carboxylate

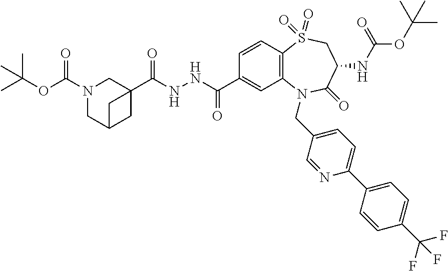 [SMILES: CC(C)(C)OC(=O)N[C@H]1CS(=O)(=O)c2ccc(C(=O)NNC(=O)C34CC(CN(C(=O)OC(C)(C)C)C3)C4)cc2N(Cc2ccc(-c3ccc(C(F)(F)F)cc3)nc2)C1=O]

The title compound was prepared from tert-butyl N-[(3R)-7-(hydrazinecarbonyl)-1,1,4-trioxo-5-[[6-[4-(trifluoromethyl)phenyl]-3-pyridyl]methyl]-2,3-dihydro-1λ⁶,5-benzothiazepin-3-yl]carbamate (150 mg, 0.24 mmol) and 3-tert-butoxycarbonyl-3-azabicyclo[3.1.1]heptane-1-carboxylic acid (75.9 mg, 0.31 mmol, 1.3 eq. CAS 1000931-22-5) in analogy to general procedure 4a and was obtained as light yellow solid (310 mg, 0.39 mmol, 74% yield). MS (ESI): 843.4 [M+H]⁺.

### Step f) tert-butyl 1-[5-[(3R)-3-(tert-butoxycarbonylamino)-1,1,4-trioxo-5-[[6-[4-(trifluoromethyl)phenyl]-3-pyridyl]methyl]-2,3-dihydro-1λ⁶, 5-benzothiazepin-7-yl]-1,3,4-oxadiazol-2-yl]-3-azabicyclo[3.1.1]heptane-3-carboxylate

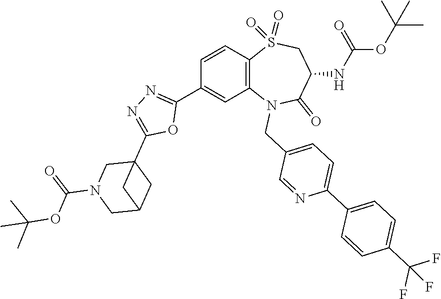 [SMILES: CC(C)(C)OC(=O)N[C@H]1CS(=O)(=O)c2ccc(-c3nnc(C45CC(CN(C(=O)OC(C)(C)C)C4)C5)o3)cc2N(Cc2ccc(-c3ccc(C(F)(F)F)cc3)nc2)C1=O]

The title compound was prepared from tert-butyl 1-[[[(3R)-3-(tert-butoxycarbonylamino)-1,1,4-trioxo-5-[[6-[4-(trifluoromethyl)phenyl]-3-pyridyl]methyl]-2,3-dihydro-1λ⁶,5-benzothiazepine-7-carbonyl]amino]carbamoyl]-3-azabicyclo[3.1.1]heptane-3-carboxylate (330 mg, 0.37 mmol) in analogy to general procedure 5a and was obtained as light yellow solid (150 mg, 0.18 mmol, 46% yield). MS (ESI): 825.1 [M+H]⁺.

### Step g) (3R)-3-amino-7-[5-(3-azabicyclo[3.1.]]heptan-1-yl)-1,3,4-oxadiazol-2-yl]-1,1-dioxo-5-[[6-[4-(trifluoromethyl)phenyl]-3-pyridyl]methyl]-2,3-dihydro-1λ⁶,5-benzothiazepin-4-one

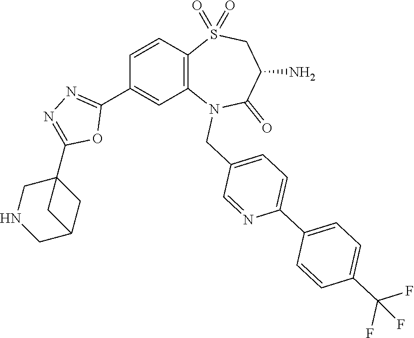 [SMILES: N[C@H]1CS(=O)(=O)c2ccc(-c3nnc(C45CNCC(C4)C5)o3)cc2N(Cc2ccc(-c3ccc(C(F)(F)F)cc3)nc2)C1=O]

The title compound was prepared from tert-butyl 1-[5-[(3R)-3-(tert-butoxycarbonylamino)-1,1,4-trioxo-5-[[6-[4-(trifluoromethyl)phenyl]-3-pyridyl]methyl]-2,3-dihydro-1λ⁶,5-benzothiazepin-7-yl]-1,3,4-oxadiazol-2-yl]-3-azabicyclo[3.1.1]heptane-3-carboxylate (145 mg, 0.18 mmol)) in analogy to general procedure 11c and was obtained as light yellow solid (70 mg, 0.1 mmol, 54% yield). MS (ESI): 625.3 [M+H]⁺.

### Step h) methyl 1-[5-[(3R)-3-amino-1,1,4-trioxo-5-[[6-[4-(trifluoromethyl)phenyl]-3-pyridyl]methyl]-2,3-dihydro-1λ⁶, 5-benzothiazepin-7yl]-1,3,4-oxadiazol-2-yl]-3-azabicyclo[3.1.1]heptane-3-carboxylate

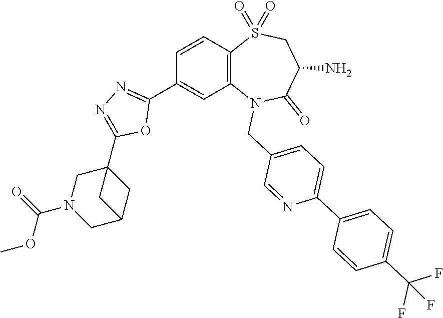 [SMILES: COC(=O)N1CC2CC(c3nnc(-c4ccc5c(c4)N(Cc4ccc(-c6ccc(C(F)(F)F)cc6)nc4)C(=O)[C@@H](N)CS5(=O)=O)o3)(C2)C1]

To a solution (3R)-3-amino-7-[5-(3-azabicyclo[3.1.1]heptan-1-yl)-1,3,4-oxadiazol-2-yl]-1,1-dioxo-5-[[6-[4-(trifluoromethyl)phenyl]-3-pyridyl]methyl]-2,3-dihydro-1λ⁶,5-benzothiazepin-4-one (60.0 mg, 0.08 mmol, 1.0 eq) and N,N-diisopropylethylamine (42.26 mg, 0.33 mmol, 4.0 eq) in D F (1 mL) was added dimethyl dicarbonate (11 ng, 0.08 mmol, 1.0 eq) at RT and stirred for-2 h. The reaction mixture was poured into water (10 mL) and EtOAc (20 ml) was added. The phases were separated and the aqueous phase was extracted with EtOAc (2×). The combined organic phase was washed with brine (5 mL), dried over anhydrous sodium sulfate, filtered, and concentrated under reduced pressure. The remaining residue was purified by prep-HPLC (HCs) to give the title compound as white solid, as hydrochloride salt (36.5 mg, 0.05 mmol, 60% yield). MS (ESI): 683.3 [M+H]⁺

The examples of the following table were prepared in analogy to Example 58, using the appropriate carboxylic acid and benzyl halide building block with the indicated general procedure.

| | | | Building | MS, |
|---|---|---|---|---|
| | | | Block and | ESI: |
| Ex. | Structure | Systematic Name | synthesis step | m/z |
| 59 | 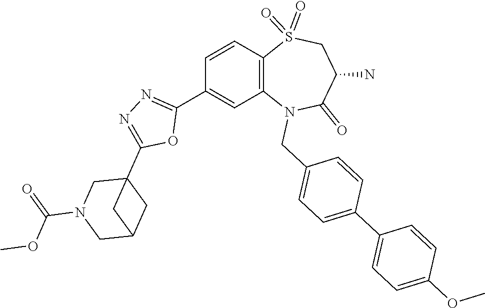 | methyl 1-[5-[(3R)-3- amino-5-[[4-(4- methoxyphenyl) phenyl]methyl]-1,1,4- trioxo-2,3-dihydro- 1λ⁶,5- benzothiazepin-7- yl]-1,3,4-oxadiazol- 2-yl]-3- azabicyclo[3.1.1] heptane-3-carboxylate | 4- (bromomethyl)- 4′-methoxy- 1,1′-biphenyl (CAS 20854- 61-9) | 644.2 [M + H]⁺ |
| 60 | 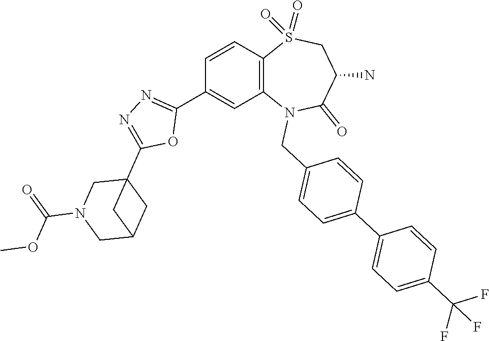 [SMILES: COC(=O)N1CC2CC(c3nnc(-c4ccc5c(c4)N(Cc4ccc(-c6ccc(C(F)(F)F)cc6)cc4)C(=O)[C@@H](N)CS5(=O)=O)o3)(C2)C1] | methyl 1-[5-[(3R)-3- amino-1,1,4-trioxo- 5-[[4-[4- (trifluoromethyl) phenyl]phenyl]methyl]- 2,3-dihydro-1λ⁶,5- benzothiazepin-7- yl]-1,3,4-oxadiazol- 2-yl]-3- azabicyclo[3.1.1] heptane-3-carboxylate | 4- (Bromomethyl)- 4′- (trifluoro- methyl)-1,1′- biphenyl (CAS 613241-14-8) | 682.2 [M + H]⁺ |
| 61 | 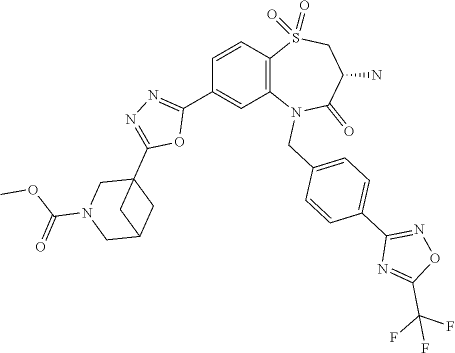 [SMILES: COC(=O)N1CC2CC(c3nnc(-c4ccc5c(c4)N(Cc4ccc(-c6noc(C(F)(F)F)n6)cc4)C(=O)[C@@H](N)CS5(=O)=O)o3)(C2)C1] | methyl 1-[5-[(3R)-3- amino-1,1,4-trioxo- 5-[[4-[5- (trifluoromethyl)- 1,2,4-oxadiazol-3- yl]phenyl]methyl]- 2,3-dihydro-1λ⁶,5- benzothiazepin-7- yl]-1,3,4-oxadiazol- 2-yl]-3- azabicyclo[3.1.1] heptane-3-carboxylate | 3-[4- (bromomethyl) phenyl]-5- (trifluoro- methyl)-1,2,4- oxadiazole (CAS 2093101-98- 3) | 674.1 [M + H]⁺ |
| 62 | 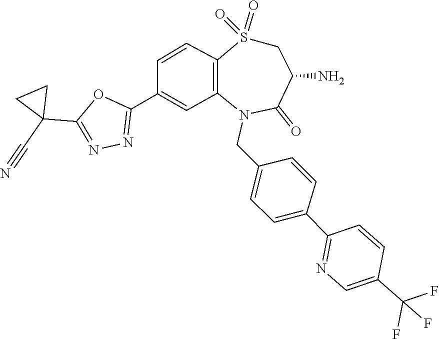 [SMILES: N#CC1(c2nnc(-c3ccc4c(c3)N(Cc3ccc(-c5ccc(C(F)(F)F)cn5)cc3)C(=O)[C@@H](N)CS4(=O)=O)o2)CC1] | 1-[5-[(3R)-3-amino- 1,1,4-trioxo-5-[[4- [5-(trifluoromethyl)- 2- pyridyl]phenyl] methyl]-2,3-dihydro- 1λ⁶,5- benzothiazepin-7- yl]-1,3,4-oxadiazol- 2- yl]cyclopropanecar- bonitrile (*) | Step a - f: 2- [4- (bromomethyl) phenyl]-5- (trifluoromethyl) pyridine (CAS 1056641-21- 4, step b); 1- cyanocyclopro- panecarboxylic acid (CAS 6914-79-0, step e) | 595.0 [M + H]⁺ |
| 158 | 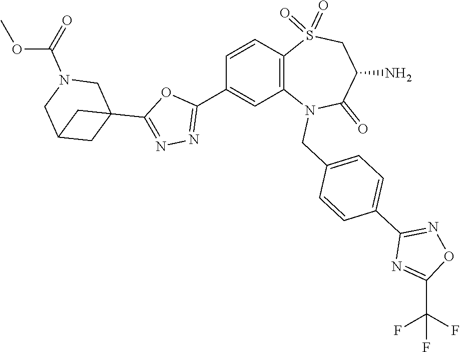 [SMILES: COC(=O)N1CC2CC(c3nnc(-c4ccc5c(c4)N(Cc4ccc(-c6noc(C(F)(F)F)n6)cc4)C(=O)[C@@H](N)CS5(=O)=O)o3)(C2)C1] | methyl 1-[5-[(3R)-3- amino-1,1,4-trioxo- 5-[[4-[5- (trifluoromethyl)- 1,2,4-oxadiazol-3- yl]phenyl]methyl]- 2,3-dihydro-1λ⁶,5- benzothiazepin-7- yl]-1,3,4-oxadiazol- 2-yl]-3- azabicyclo[3.1.1] heptane-3-carboxylate | 3-[4- (bromomethyl) phenyl]-5- (trifluoro- methyl)-1,2,4- oxadiazole (CAS 2093101-98- 3) | 674.4 [M + H]⁺ |
| 159 | 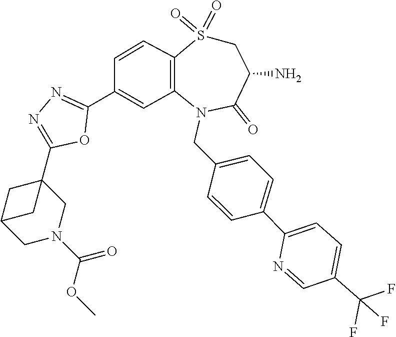 [SMILES: COC(=O)N1CC2CC(c3nnc(-c4ccc5c(c4)N(Cc4ccc(-c6ccc(C(F)(F)F)cn6)cc4)C(=O)[C@@H](N)CS5(=O)=O)o3)(C2)C1] | methyl 1-[5-[(3R)-3- amino-1,1,4-trioxo- 5-[[4-[5- (trifluoromethyl)-2- pyridyl]phenyl] methyl]-2,3-dihydro- 1λ⁶,5- benzothiazepin-7- yl]-1,3,4-oxadiazol- 2-yl]-3- azabicyclo[3.1.1 ] heptane-3-carboxylate | 2-[4- (bromomethyl) phenyl]-5- (trifluoromethyl) pyridine (CAS 1056641-21- 4) | 683.3 [M + H]⁺ |

(*) as hydrochloride salt

### Example 63

### (3R)-3-amino-7-[5-[2-(hydroxymethyl)tetrahydrofuran-2-yl]-1,3,4-oxadiazol-2-yl]-5-[[4-(4-methoxyphenyl)phenyl]methyl]-1,1-dioxo-2,3-dihydro-1λ⁶,5-benzothiazepin-4-one

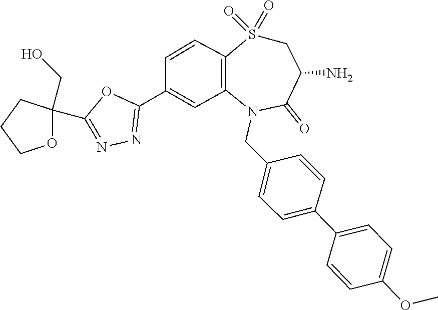 [SMILES: COc1ccc(-c2ccc(CN3C(=O)[C@@H](N)CS(=O)(=O)c4ccc(-c5nnc(C6(CO)CCCO6)o5)cc43)cc2)cc1]

### Step a) tert-butyl N-[(3R)-7-[[[2-(hydroxymethyl)tetrahydrofuran-2-carbonyl]amino]carbamoyl]-1,1,4-trioxo-3,5-dihydro-2H-1λ⁶, 5-benzothiazepin-3-yl]carbamate

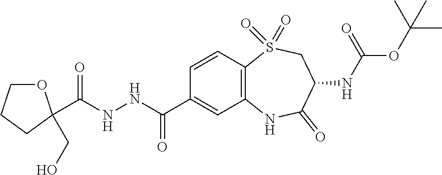 [SMILES: CC(C)(C)OC(=O)N[C@H]1CS(=O)(=O)c2ccc(C(=O)NNC(=O)C3(CO)CCCO3)cc2NC1=O]

The title compound was prepared in analogy to general procedure 4a from (3R)-3-(tert-butoxycarbonylamino)-1,1,4-trioxo-3,5-dihydro-2H-1λ⁶,5-benzothiazepine-7-carboxylic acid (Example 32, step a) (260 mg, 676.4 μmol, 1.0 eq) and 2-methyloltetrahydrofuran-2-carboxylic acid (CAS:61449-65-8) and was obtained as white solid (280 mg, 53% yield). MS (ESI): 413.1 [M-Boc+H]⁺

### Step b) tert-butyl N-[(3R)-7-[[[2-[[tert-butyl(dimethyl)silyl]oxymethyl]tetrahydrofuran-2-carbonyl]amino]carbamoyl]-1,1,4-trioxo-3,5-dihydro-2H-1λ⁶,5-benzothiazepin-3-yl]carbamate

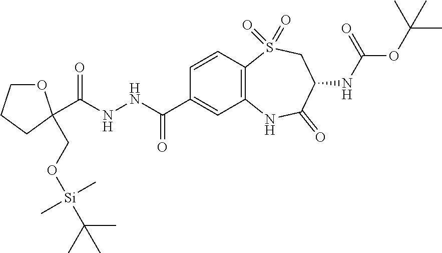 [SMILES: CC(C)(C)OC(=O)N[C@H]1CS(=O)(=O)c2ccc(C(=O)NNC(=O)C3(CO[Si](C)(C)C(C)(C)C)CCCO3)cc2NC1=O]

tert-butyl N-[(3R)-7-[[[2-(hydroxymethyl)tetrahydrofuran-2-carbonyl]amino]carbamoyl]-1,1,4-trioxo-3,5-dihydro-2H-1λ⁶,5-benzothiazepin-3-yl]carbamate (280 mg, 546.31 μmol, 1.0 eq) was stirred with TBDMS-Cl (123.51 mg, 819.46 μmol, 1.5 eq) and imidazole (92.98 mg, 1.37 mmol, 2.5 eq) in DCM (5.45 mL) at RT for 16 h. The reaction was directly concentrated and purified by column chromatography on silica gel (0-80% EtOAc in heptane) to afford the title compound as a white solid (167 mg, 49%). MS (ESI): 571.2 [M-isobutene+H]⁺

### Step c) tert-butyl N-[(3R)-7-[5-[2-[[tert-butyl(dimethyl)silyl]oxymethyl]tetrahydrofuran-2-yl]-1,3,4-oxadiazol-2-yl]-1,1,4-trioxo-3,5-dihydro-2H-1λ⁶,5-benzothiazepin-3-yl]carbamate

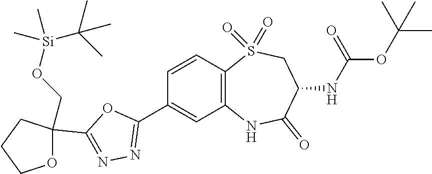 [SMILES: CC(C)(C)OC(=O)N[C@H]1CS(=O)(=O)c2ccc(-c3nnc(C4(CO[Si](C)(C)C(C)(C)C)CCCO4)o3)cc2NC1=O]

The title compound was prepared in analogy to general procedure 5b from tert-butyl N-[(3R)-7-[[[2-[[tert-butyl(dimethyl)silyl]oxymethyl]tetrahydrofuran-2-carbonyl]amino]carbamoyl]-1,1,4-trioxo-3,5-dihydro-2H-1λ⁶,5-benzothiazepin-3-yl]carbamate (160 mg, 0.255 mmol) and was obtained as white solid (135 mg, 87% yield). MS (ESI): 609.2 [M+H]⁺

### Step d) tert-butyl N-[(3R)-7-[5-[2-[[tert-butyl(dimethyl)silyl]oxymethyl]tetrahydrofuran-2-yl]-1,3,4-oxadiazol-2-yl]-5-[[4-(4-methoxyphenyl)phenyl]methyl]-1,1,4-trioxo-2,3-dihydro-1λ⁶,5-benzothiazepin-3-yl]carbamate

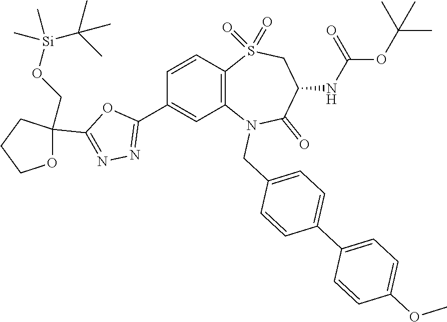 [SMILES: COc1ccc(-c2ccc(CN3C(=O)[C@@H](NC(=O)OC(C)(C)C)CS(=O)(=O)c4ccc(-c5nnc(C6(CO[Si](C)(C)C(C)(C)C)CCCO6)o5)cc43)cc2)cc1]

The title compound was prepared from tert-butyl N-[(3R)-7-[5-[2-[[tert-butyl(dimethyl)silyl]oxymethyl]tetrahydrofuran-2-yl]-1,3,4-oxadiazol-2-yl]-1,1,4-trioxo-3,5-dihydro-2H-1λ⁶,5-benzothiazepin-3-yl]carbamate (50 mg, 0.082 mmol, 1.0 eq) and 4-(bromomethyl)-4′-methoxy-1,1′-biphenyl (29.6 mg, 0.11 mmol, 1.3 eq, CAS: 20854-61-9) in analogy to general procedure 1a and was obtained as white solid (57.4 mg, 86% yield). MS (ESI): 805.4 [M+H]⁺

### Step e) (3R)-3-amino-7-[5-[2-(hydroxymethyl)tetrahydrofuran-2-yl]-1,3,4-oxadiazol-2-yl]-5-[[4-(4-methoxyphenyl)phenyl]methyl]-1,1-dioxo-2,3-dihydro-1λ⁶, 5-benzothiazepin-4-one

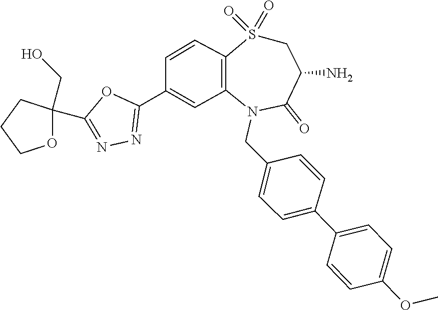 [SMILES: COc1ccc(-c2ccc(CN3C(=O)[C@@H](N)CS(=O)(=O)c4ccc(-c5nnc(C6(CO)CCCO6)o5)cc43)cc2)cc1]

tert-butyl N-[(3R)-7-[5-[2-[[tert-butyl(dimethyl)silyl]oxymethyl]tetrahydrofuran-2-yl]-1,3,4-oxadiazol-2-yl]-5-[[4-(4-methoxyphenyl)phenyl]methyl]-1,1,4-trioxo-2,3-dihydro-1λ⁶,5-benzothiazepin-3-yl]carbamate (57.4 mg, 71.3 μmol, 1.0 eq) was dissolved in 1,1,1,3,3,3-hexafluoro-2-propanol (6.43 mL) and 1 M HCl in Et₂O (165.12 mg, 221.04 uL, 221.04 μmol, 3.1 eq) was added at RT and stirred for 1 h. TBAF (1M in THF, 18.6 mg, 0.071 mmol, 1.0 eq) was then added and stirring was continued for 2 h. The reaction was concentrated under reduced pressure and directly purified by reverse-phase prep. HPLC to afford the title compound as white powder (13.5 mg, 32%). MS (ESI): 591.4 [M+H]⁺.

### Example 64

### (3R)-3-amino-7-[5-(1-ethoxy-1,2,2,2-tetrafluoro-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

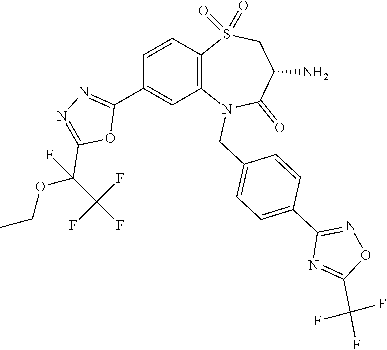 [SMILES: CCOC(F)(c1nnc(-c2ccc3c(c2)N(Cc2ccc(-c4noc(C(F)(F)F)n4)cc2)C(=O)[C@@H](N)CS3(=O)=O)o1)C(F)(F)F]

### Step a) 2-ethoxy-2,3,3,3-tetrafluoro-propanehydrazide

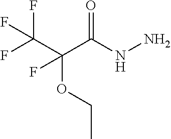

To a solution of 2-ethoxy-2,3,3,3-tetrafluoropropanoic acid (200 mg, 0.92 mmol, 1 eq, CAS 10186-67-1) in EtOH (1.5 ml) was added hydrazine monohydrate (54.25 mg, 1.1 mmol, 1.2 eq) and the mixture was heated to 80° C. for 8 h. The resulting colorless solution was concentrated under reduced pressure and dried under high vacuum to afford 2-ethoxy-2,3,3,3-tetrafluoro-propanehydrazide (119 mg, 60%) as white solid. MS (ESI): 205.1 [M+H]⁺

### Step b) tert-butyl N-[(3R)-7-[[(2-ethoxy-2,3,3,3-tetrafluoro-propanoyl)amino]carbamoyl]-4-oxo-5-[[4-[5-(trifluoromethyl)-1,2,4-oxadiazol-3-yl]phenyl]methyl]-2,3-dihydro-1,5-benzothiazepin-3-yl]carbamate

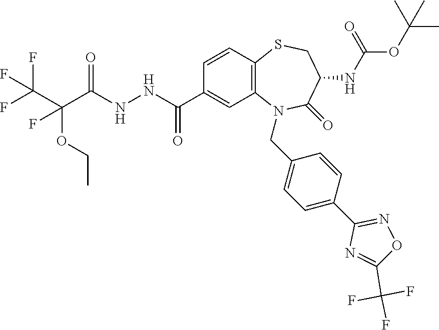 [SMILES: CCOC(F)(C(=O)NNC(=O)c1ccc2c(c1)N(Cc1ccc(-c3noc(C(F)(F)F)n3)cc1)C(=O)[C@@H](NC(=O)OC(C)(C)C)CS2)C(F)(F)F]

The title compound as prepared from (3R)-3-(tert-butoxycarbonylamino)-4-oxo-5-[[4-[5-(trifluoromethyl)-1,2,4-oxadiazol-3-yl]phenyl]methyl]-2,3-dihydro-1,5-benzothiazepine-7-carboxylic acid (145 mg, 0.257 mmol, 1 eq, Example 54, step b) and 2-ethoxy-2,3,3,3-tetrafluoro-propanehydrazide (68.16 mg, 0.334 mmol, 1.3 eq) in analogy to general procedure 4a and was obtained as yellow oil (247 mg, 99%). MS (ESI): 749.3 [M−H]

### Step c) tert-butyl N-[(3R)-7-[5-(1-ethoxy-1,2,2,2-tetrafluoro-ethyl)-1,3,4-oxadiazol-2-yl]-4-oxo-5-[[4-[5-(trifluoromethyl)-1,2,4-oxadiazol-3-yl]phenyl]methyl]-2,3-dihydro-1,5-benzothiazepin-3-yl]carbamate

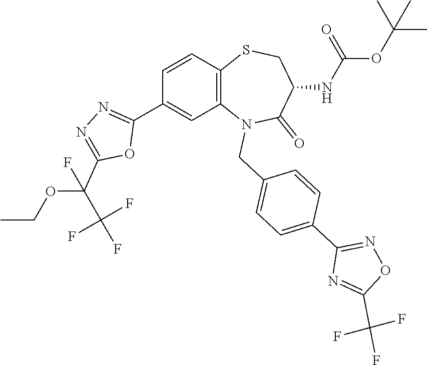 [SMILES: CCOC(F)(c1nnc(-c2ccc3c(c2)N(Cc2ccc(-c4noc(C(F)(F)F)n4)cc2)C(=O)[C@@H](NC(=O)OC(C)(C)C)CS3)o1)C(F)(F)F]

The title compound was prepared from tert-butyl N-[(3R)-7-[[(2-ethoxy-2,3,3,3-tetrafluoro-propanoyl)amino]carbamoyl]-4-oxo-5-[[4-[5-(trifluoromethyl)-1,2,4-oxadiazol-3-yl]phenyl]methyl]-2,3-dihydro-1,5-benzothiazepin-3-yl]carbamate (247 mg, 0.247 mmol) in analogy to general procedure 5b and was obtained as white (119 mg, 65%). MS (ESI): 677.3 [M-isobutene+H]⁺

### Step d) tert-butyl N-[(3R)-7-[5-(1-ethoxy-1,2,2,2-tetrafluoro-ethyl)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate

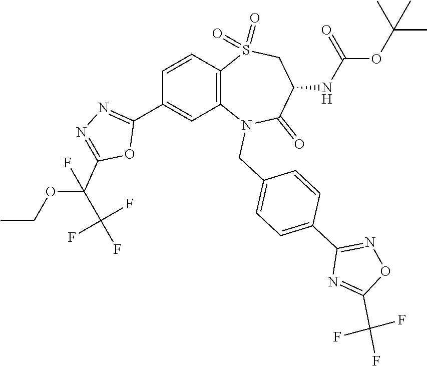 [SMILES: CCOC(F)(c1nnc(-c2ccc3c(c2)N(Cc2ccc(-c4noc(C(F)(F)F)n4)cc2)C(=O)[C@@H](NC(=O)OC(C)(C)C)CS3(=O)=O)o1)C(F)(F)F]

The title compound was prepared from tert-butyl N-[(3R)-7-[5-(1-ethoxy-1,2,2,2-tetrafluoro-ethyl)-1,3,4-oxadiazol-2-yl]-4-oxo-5-[[4-[5-(trifluoromethyl)-1,2,4-oxadiazol-3-yl]phenyl]methyl]-2,3-dihydro-1,5-benzothiazepin-3-yl]carbamate (112 mg, 0.15 mmol) in analogy to general procedure 10 and was obtained as white solid (96 mg, 83%). MS (ESI): 709.2 [M-isobutene+H]

### Step e) (3R)-3-amino-7-[5-(1-ethoxy-1,2,2,2-tetrafluoro-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one

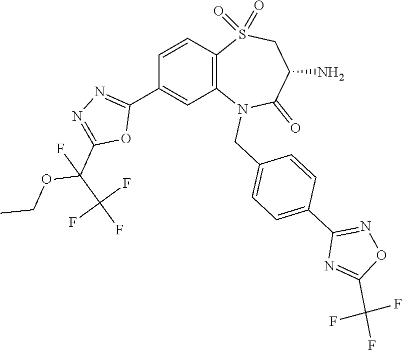 [SMILES: CCOC(F)(c1nnc(-c2ccc3c(c2)N(Cc2ccc(-c4noc(C(F)(F)F)n4)cc2)C(=O)[C@@H](N)CS3(=O)=O)o1)C(F)(F)F]

The title compound was prepared from tert-butyl N-[(3R)-7-[5-(1-ethoxy-1,2,2,2-tetrafluoro-ethyl)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (96 mg, 0.126 mmol) in analogy to general procedure 11a and was obtained as white solid, was hydrochloride (67.5 mg, 76%). MS (ESI): 665.5 [M+H]⁺

### Example 65

### ((3R)-3-amino-5-[[4-(5-cyclopropyl-1,3,4-oxadiazol-2-yl)phenyl]methyl]-1,1-dioxo-7-[5-(1,2,2,2-tetrafluoro-1-methoxy-ethyl)-1,3,4-oxadiazol-2-yl]-2,3-dihydro-1λ⁶,5-benzothiazepin-4-one

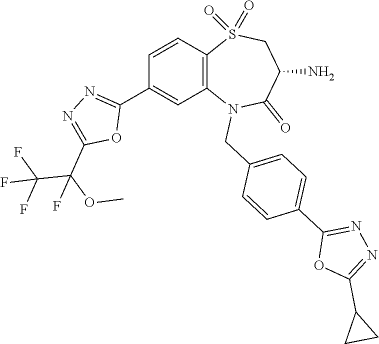 [SMILES: COC(F)(c1nnc(-c2ccc3c(c2)N(Cc2ccc(-c4nnc(C5CC5)o4)cc2)C(=O)[C@@H](N)CS3(=O)=O)o1)C(F)(F)F]

### Step a) tert-butyl N-[(3R)-4-oxo-7-[[(2,3,3,3-tetrafluoro-2-methoxy-propanoyl)amino]carbamoyl]-3,5-dihydro-2H-1,5-benzothiazepin-3-yl]carbamate

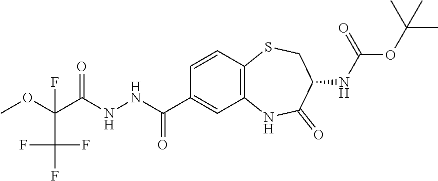 [SMILES: COC(F)(C(=O)NNC(=O)c1ccc2c(c1)NC(=O)[C@@H](NC(=O)OC(C)(C)C)CS2)C(F)(F)F]

The title compound was prepared in analogy to general procedure 4b from (3R)-3-(tert-butoxycarbonylamino)-4-oxo-3,5-dihydro-2H-1,5-benzothiazepine-7-carboxylic acid (Example 19, step a, 900 mg, 2.66 mmol, 1.0 eq) and 2,3,3,3-tetrafluoro-2-methoxy-propanehydrazide (505 mg, 2.7 mmol, 1.0 eq, Example 64, step a) and was obtained as a light yellow solid (1300 mg, 95% yield). MS (ESI) 509.2 [M−H]⁻

### Step b) tert-butyl N-[(3R)-4-oxo-7-[5-(1,2,2,2-tetrafluoro-1-methoxy-ethyl)-1,3,4-oxadiazol-2-yl]-3,5-dihydro-2H-1,5-benzothiazepin-3-yl]carbamate

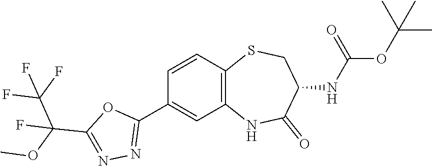 [SMILES: COC(F)(c1nnc(-c2ccc3c(c2)NC(=O)[C@@H](NC(=O)OC(C)(C)C)CS3)o1)C(F)(F)F]

The title compound was prepared in analogy to general procedure 4b from tert-butyl N-[(3R)-4-oxo-7-[[(2,3,3,3-tetrafluoro-2-methoxy-propanoyl)amino]carbamoyl]-3,5-dihydro-2H-1,5-benzothiazepin-3-yl]carbamate (400 mg, 0.78 mmol, 1.0 eq) and was obtained as a light yellow oil (560 mg, 116% yield), which was used without further purification. MS (ESI) 437.1 [M-isobutene+H]⁺

### Step c) tert-butyl N-[(3R)-1,1,4-trioxo-7-[5-(1,2,2,2-tetrafluoro-1-methoxy-ethyl)-1,3,4-oxadiazol-2-yl]-3,5-dihydro-2H-1λ⁶,5-benzothiazepin-3-yl]carbamate

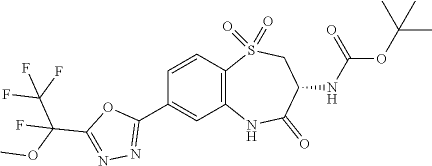 [SMILES: COC(F)(c1nnc(-c2ccc3c(c2)NC(=O)[C@@H](NC(=O)OC(C)(C)C)CS3(=O)=O)o1)C(F)(F)F]

The title compound was prepared in analogy to general procedure 10 from tert-butyl N-[(3R)-4-oxo-7-[5-(1,2,2,2-tetrafluoro-1-methoxy-ethyl)-1,3,4-oxadiazol-2-yl]-3,5-dihydro-2H-1,5-benzothiazepin-3-yl]carbamate (280 mg, 0.57 mmol, 1. eq) and was obtained as a light yellow solid (500 mg) containing the title compound. MS (ESI) 468.9 [M-isobutene+H]⁺

### Step d) tert-butyl N-[(3R)-5-[[4-(5-cyclopropyl-1,3,4-oxadiazol-2-yl)phenyl]methyl]-1,1,4-trioxo-7-[5-(1,2,2,2-tetrafluoro-1-methoxy-ethyl)-1,3,4-oxadiazol-2-yl]-2,3-dihydro-1λ⁶, 5-benzothiazepin-3-yl]carbamate

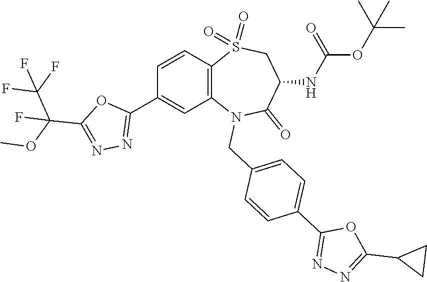 [SMILES: COC(F)(c1nnc(-c2ccc3c(c2)N(Cc2ccc(-c4nnc(C5CC5)o4)cc2)C(=O)[C@@H](NC(=O)OC(C)(C)C)CS3(=O)=O)o1)C(F)(F)F]

The title compound was prepared in analogy to general procedure 1a from tert-butyl N-[(3R)-1,1,4-trioxo-7-[5-(1,2,2,2-tetrafluoro-1-methoxy-ethyl)-1,3,4-oxadiazol-2-yl]-3,5-dihydro-2H-1λ⁶,5-benzothiazepin-3-yl]carbamate (15 mg, 0.029 mmol, 1 eq) and Intermediate 14 (10.4 mg, 0.037 mmol, 1.3 eq) and was obtained as a white powder (11 mg, 53% yield). MS (ESI): 723.2 [M-isobutene+H]⁺

### Step e) (3R)-3-amino-1,1-dioxo-7-[5-(1,2,2,2-tetrafluoro-1-methoxy-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one

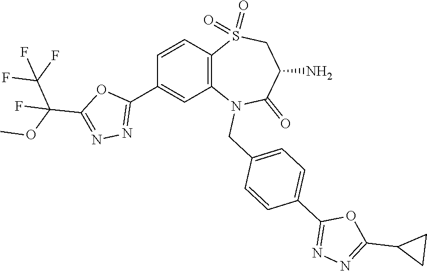 [SMILES: COC(F)(c1nnc(-c2ccc3c(c2)N(Cc2ccc(-c4nnc(C5CC5)o4)cc2)C(=O)[C@@H](N)CS3(=O)=O)o1)C(F)(F)F]

The title compound was prepared in analogy to general procedure 11c from tert-butyl N-[(3R)-5-[[4-(5-cyclopropyl-1,3,4-oxadiazol-2-yl)phenyl]methyl]-1,1,4-trioxo-7-[5-(1,2,2,2-tetrafluoro-1-methoxy-ethyl)-1,3,4-oxadiazol-2-yl]-2,3-dihydro-1λ⁶,5-benzothiazepin-3-yl]carbamate (11 mg, 0.015 mmol) and was obtained as white powder, as hydrochloride salt (8.4 mg, 84% yield). MS (ESI): 623.1 [M+H]⁺

### Example 66 of the Following Table was Prepared in Analogy to Example 65 Using the Appropriate Benzyl Bromide Building Block

| | | | | MS, |
|---|---|---|---|---|
| | | | Building | ESI: |
| Ex. | Structure | Systematic Name | Blocks | m/z |
| 66 | 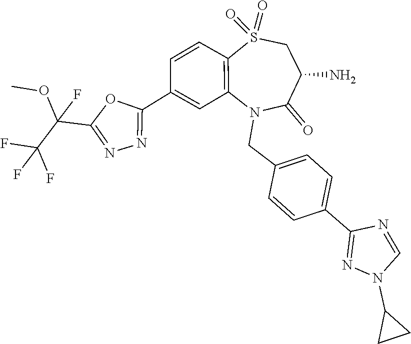 [SMILES: COC(F)(c1nnc(-c2ccc3c(c2)N(Cc2ccc(-c4ncn(C5CC5)n4)cc2)C(=O)[C@@H](N)CS3(=O)=O)o1)C(F)(F)F] | (3R)-3-amino-5-[[4- (1-cyclopropyl-1,2,4- triazol-3- yl)phenyl]methyl]- 1,1-dioxo-7-[5- (1,2,2,2-tetrafluoro-1- methoxy-ethyl)-1,3,4- oxadiazol-2-yl]-2,3- dihydro-1λ⁶,5- benzothiazepin-4-one (*) | Intermediate 15 | 622.3 [M + H]⁺ |
| 67 | 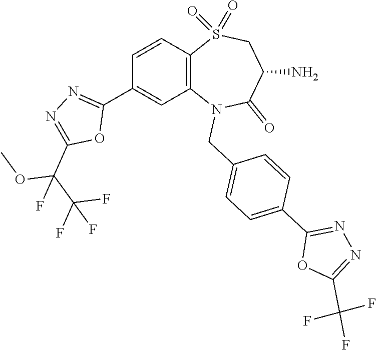 [SMILES: COC(F)(c1nnc(-c2ccc3c(c2)N(Cc2ccc(-c4nnc(C(F)(F)F)o4)cc2)C(=O)[C@@H](N)CS3(=O)=O)o1)C(F)(F)F] | (3R)-3-amino-1,1- dioxo-7-[5-(1,2,2,2- tetrafluoro-1- methoxy-ethyl)-1,3,4- oxadiazol-2-yl]-5-[[4- [5-(trifluoromethyl)- 1,3,4-oxadiazol-2- yl]phenyl]methyl]- 2,3-dihydro-1λ⁶,5- benzothiazepin-4-one (*) | Intermediate 4 | 651.4 [M + H]⁺ |

* as hydrochloride salt

### Example 68

### (3R)-3-amino-7-[5-(4,4-difluoro-1-piperidyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

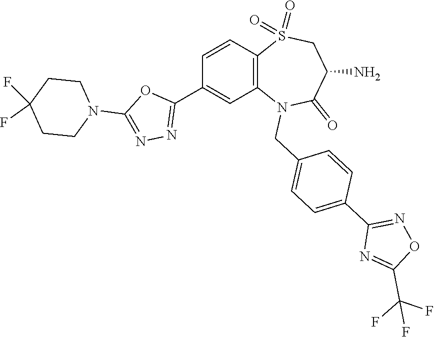 [SMILES: N[C@H]1CS(=O)(=O)c2ccc(-c3nnc(N4CCC(F)(F)CC4)o3)cc2N(Cc2ccc(-c3noc(C(F)(F)F)n3)cc2)C1=O]

### Step a) tert-butyl N-[(3R)-1,1,4-trioxo-7-(2-oxo-3H-1,3,4-oxadiazol-5-yl)-3,5-dihydro-2H-1λ⁶,5-benzothiazepin-3-yl]carbamate

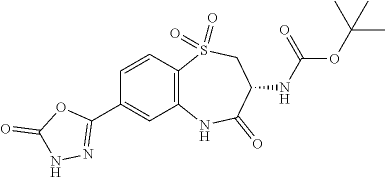 [SMILES: CC(C)(C)OC(=O)N[C@H]1CS(=O)(=O)c2ccc(-c3n[nH]c(=O)o3)cc2NC1=O]

To a solution of tert-butyl N-[(3R)-7-(hydrazinecarbonyl)-1,1,4-trioxo-3,5-dihydro-2H-1λ⁶,5-benzothiazepin-3-yl]carbamate (160 mg, 0.416 mmol, 1.0 eq, Example 7, step b) in THF (4.16 mL) was added CDI (80.99 mg, 0.499 mmol, 1.2 eq) and triethylamine (69.62 uL, 0.499 mmol, 1.2 eq). The solution was stirred at RT for 3 h. The reaction mixture was poured on water and 1N HCl was added to reach pH=3.0. The mixture was extracted with EtOAc three times. The combined organic layers were washed once with brine, dried over sodium sulfate, filtered and the solvent evaporated under reduced pressure. The remaining crude was purified by column chromatography on silica gel (10-70% EtOAc in heptane) to give the title compound as light yellow powder (183 mg, 87%). MS (ESI): 409.1 [M−H]

### Step b) tert-butyl N-[(3R)-7-[5-(4,4-difluoro-1-piperidyl)-], 3,4-oxadiazol-2-yl]-1,1,4-trioxo-3,5-dihydro-2H-1λ⁶,5-benzothiazepin-3-yl]carbamate

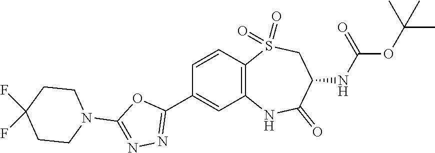 [SMILES: CC(C)(C)OC(=O)N[C@H]1CS(=O)(=O)c2ccc(-c3nnc(N4CCC(F)(F)CC4)o3)cc2NC1=O]

The title compound was prepared from tert-butyl N-[(3R)-1,1,4-trioxo-7-(2-oxo-3H-1,3,4-oxadiazol-5-yl)-3,5-dihydro-2H-1λ⁶,5-benzothiazepin-3-yl]carbamate (33 mg, 0.06 mmol) and difluoropiperidine hydrochloride (171 mg, 1.08 mmol, 2.5 eq, CAS 144230-52-4) in analogy to general procedure 13 and was obtained as yellow solid (192 mg, 59%). MS (ESI): 514.2 [M+H]⁺

### Step c) tert-butyl N-[(3R)-7-[5-(4,4-difluoro-1-piperidyl)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶, 5-benzothiazepin-3-yl]carbamate

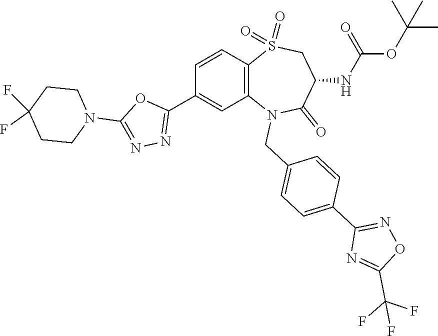 [SMILES: CC(C)(C)OC(=O)N[C@H]1CS(=O)(=O)c2ccc(-c3nnc(N4CCC(F)(F)CC4)o3)cc2N(Cc2ccc(-c3noc(C(F)(F)F)n3)cc2)C1=O]

The title compound was prepared from tert-butyl N-[(3R)-7-[5-(4,4-difluoro-1-piperidyl)-1,3,4-oxadiazol-2-yl]-1,1,4-trioxo-3,5-dihydro-2H-1λ⁶,5-benzothiazepin-3-yl]carbamate (20.5 mg, 0.04 mmol, 1.0 eq) and 3-[4-(bromomethyl)phenyl]-5-(trifluoromethyl)-1,2,4-oxadiazole (15.94 mg, 0.052 mmol, 1.3 eq, CAS 2093101-98-3) in analogy to general procedure 1a and was obtained as white solid (15.1 mg, 51%). MS (ESI): 740.4 [M+H]⁺

### Step d) (3R)-3-amino-7-[5-(4,4-difluoro-1-piperidyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

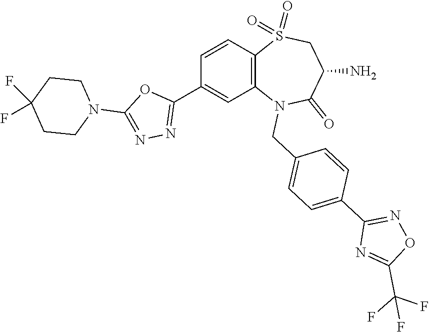 [SMILES: N[C@H]1CS(=O)(=O)c2ccc(-c3nnc(N4CCC(F)(F)CC4)o3)cc2N(Cc2ccc(-c3noc(C(F)(F)F)n3)cc2)C1=O]

The title compound was prepared from tert-butyl N-[(3R)-7-[5-(4,4-difluoro-1-piperidyl)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (15.1 mg, 0.02 mmol) in analogy to general procedure 11a and was obtained as white solid, as hydrochloride salt (3.3 mg, 24%). MS (ESI): 640.5 [M+H]⁺

The examples of the following table were prepared in analogy to Example 68, using the appropriate benzyl halide or amine building block.

| | | | Building Block | MS, |
|---|---|---|---|---|
| | | | (synthesis | ESI: |
| Ex. | Structure | Systematic Name | step) | m/z |
| 69 | 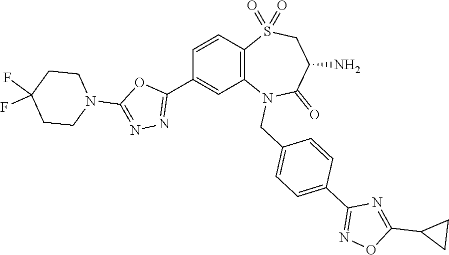 [SMILES: N[C@H]1CS(=O)(=O)c2ccc(-c3nnc(N4CCC(F)(F)CC4)o3)cc2N(Cc2ccc(-c3noc(C4CC4)n3)cc2)C1=O] | (3R)-3-amino-5-[[4-(5- cyclopropyl-1,2,4- oxadiazol-3- yl)phenyl]methyl]-7-[5- (4,4-difluoro-1- piperidyl)-1,3,4- oxadiazol-2-yl]-1,1- dioxo-2,3-dihydro- 1λ⁶,5-benzothiazepin-4- one (*) | Intermediate 16 | 612.3 [M + H]⁺ |
| 70 | 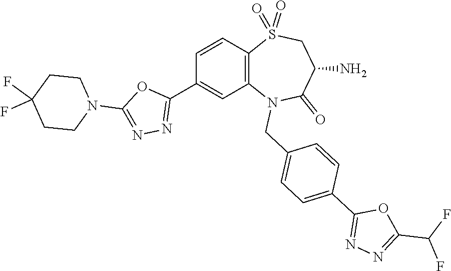 [SMILES: N[C@H]1CS(=O)(=O)c2ccc(-c3nnc(N4CCC(F)(F)CC4)o3)cc2N(Cc2ccc(-c3nnc(C(F)F)o3)cc2)C1=O] | (3R)-3-amino-5-[[4-[5- (difluoromethyl)-1,3,4- oxadiazol-2-yl]phenyl] methyl]-7-[5-(4,4-difluoro- 1-piperidyl)- 1,3,4-oxadiazol-2-yl]-1,1- dioxo- 2,3-dihydro-1λ⁶,5- benzothiazepin-4-one (*) | Intermediate 17 | 622.2 [M + H]⁺ |
| 71 | 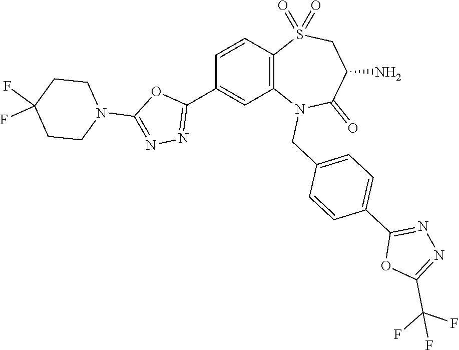 [SMILES: N[C@H]1CS(=O)(=O)c2ccc(-c3nnc(N4CCC(F)(F)CC4)o3)cc2N(Cc2ccc(-c3nnc(C(F)(F)F)o3)cc2)C1=O] | (3R)-3-amino-7-[5-(4,4- difluoro-1-piperidyl)-1,3,4- oxadiazol-2- yl]-1,1-dioxo-5-[[4-[5- (trifluoromethyl)-1,3,4- oxadiazol-2-yl]phenyl] methyl]- 2,3-dihydro-1λ⁶,5- benzothiazepin-4-one (*) | 2-[4- (bromomethyl) phenyl]-5- (trifluoromethyl)- 1,3,4- oxadiazole (CAS 2750234-21-8) | 640.2 [M + H]⁺ |
| 160 | 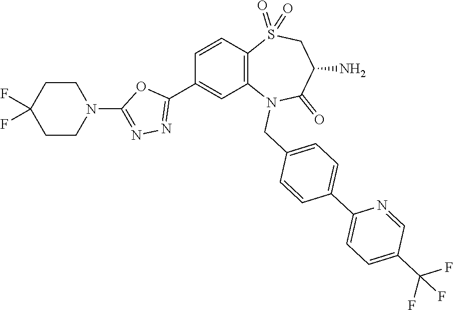 [SMILES: N[C@H]1CS(=O)(=O)c2ccc(-c3nnc(N4CCC(F)(F)CC4)o3)cc2N(Cc2ccc(-c3ccc(C(F)(F)F)cn3)cc2)C1=O] | (3R)-3-amino-7-[5-(4,4- difluoro-1-piperidyl)-1,3,4- oxadiazol-2-yl]-1,1-dioxo- 5-[[4-[5-(trifluoromethyl)-2- pyridyl]phenyl]methyl]-2,3- dihydro-1λ⁶,5- benzothiazepin-4-one (*) | 2-[4- (bromomethyl) phenyl]- 5- (trifluoromethyl) pyridine (CAS 1056641-21-4) | 649.1 [M + H]⁺ |
| 161 | 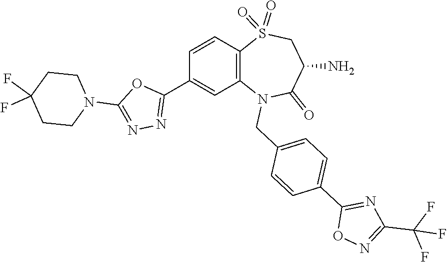 [SMILES: N[C@H]1CS(=O)(=O)c2ccc(-c3nnc(N4CCC(F)(F)CC4)o3)cc2N(Cc2ccc(-c3nc(C(F)(F)F)no3)cc2)C1=O] | (3R)-3-amino-7-[5-(4,4- difluoro-1-piperidyl)-1,3,4- oxadiazol-2-yl]-1,1-dioxo- 5-[[4-[3-(trifluoromethyl)- 1,2,4-oxadiazol-5- yl]phenyl]methyl]-2,3- dihydro-1λ⁶,5- benzothiazepin-4-one (*) | Intermediate 7 | 640.3 [M + H]⁺ |
| 162 | 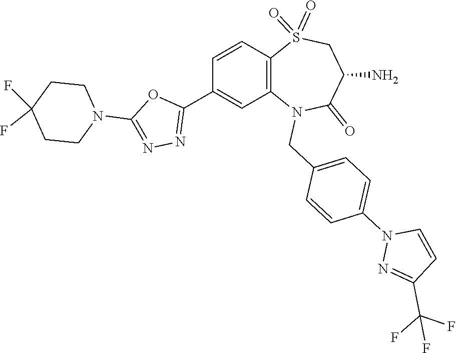 [SMILES: N[C@H]1CS(=O)(=O)c2ccc(-c3nnc(N4CCC(F)(F)CC4)o3)cc2N(Cc2ccc(-n3ccc(C(F)(F)F)n3)cc2)C1=O] | (3R)-3-amino-7-[5-(4,4- difluoro-1-piperidyl)-1,3,4- oxadiazol-2-yl]-1,1-dioxo- 5-[[4-[3- (trifluoromethyl)pyrazol-1- yl]phenyl]methyl]-2,3- dihydro-1λ⁶,5- benzothiazepin-4-one (*) | 1-[4- (Bromomethyl) phenyl]-4- (trifluoromethyl)- 1H- pyrazole (CAS 1928384-61-5) | 638.3 [M + H]⁺ |
| 163 | 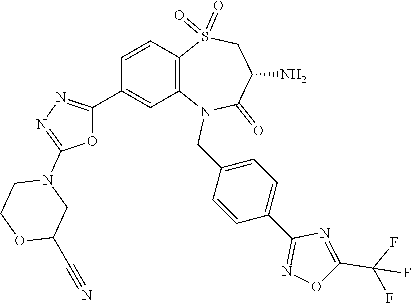 [SMILES: N#CC1CN(c2nnc(-c3ccc4c(c3)N(Cc3ccc(-c5noc(C(F)(F)F)n5)cc3)C(=O)[C@@H](N)CS4(=O)=O)o2)CCO1] | 4-[5-[(3R)-3-amino-1,1,4- trioxo-5-[4-[5- (trifluoromethyl)-1,2,4- oxadiazol-3- yl]phenyl]methyl]-2,3- dihydro-1λ⁶,5- benzothiazepin-7-yl]-1,3,4- oxadiazol-2-yl]morpholine- 2-carbonitrile | morpholine-2- carbonitrile (CAS 1205751-07-0) | 631.3 [M + H]⁺ |
| 164 | 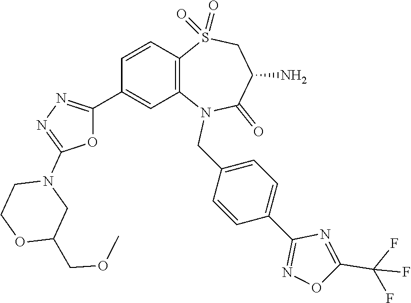 [SMILES: COCC1CN(c2nnc(-c3ccc4c(c3)N(Cc3ccc(-c5noc(C(F)(F)F)n5)cc3)C(=O)[C@@H](N)CS4(=O)=O)o2)CCO1] | (3R)-3-amino-7-[5-[2- (methoxymethyl)morpholin- 4-yl]-1,3,4-oxadiazol-2-yl]- 1,1-dioxo-5-[4-[5- (trifluoromethyl)-1,2,4- oxadiazol-3- yl]phenyl]methyl]-2,3- dihydro-1λ⁶,5- benzothiazepin-4-one (*) | 2- (methoxymethyl) morpholine (CAS 156121-15-2) | 650.3 [M + H]⁺ |

(*) as hydrochloride salt

### Example 72

### (3R)-3-amino-7-[5-[(3,3-difluoro-1-methyl-cyclobutyl)amino]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

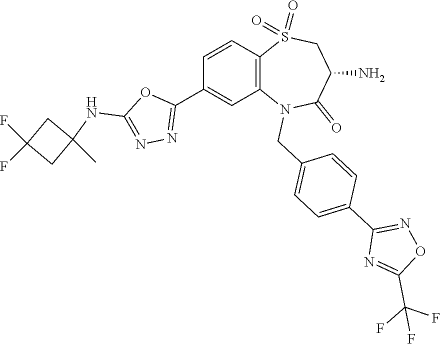 [SMILES: CC1(Nc2nnc(-c3ccc4c(c3)N(Cc3ccc(-c5noc(C(F)(F)F)n5)cc3)C(=O)[C@@H](N)CS4(=O)=O)o2)CC(F)(F)C1]

### Step a) tert-butyl N-[(3R)-7-(5-amino-1,3,4-oxadiazol-2-yl)-4-oxo-3,5-dihydro-2H-1,5-benzothiazepin-3-yl]carbamate

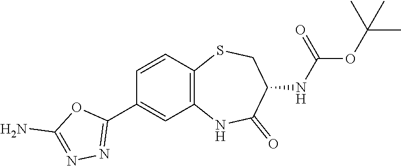 [SMILES: CC(C)(C)OC(=O)N[C@H]1CSc2ccc(-c3nnc(N)o3)cc2NC1=O]

A suspension of tert-butyl N-[(3R)-7-(hydrazinecarbonyl)-4-oxo-3,5-dihydro-2H-1,5-benzothiazepin-3-yl]carbamate (350 mg, 0.97 mmol, 1.0 eq, Example 19, step b), cyanogen bromide (318.8 mg, 2.92 mmol, 3.0 eq) and sodium bicarbonate (245.3 mg, 2.92 mmol, 3.0 eq) in 1,4-dioxane (6 mL) and water (4 mL) was stirred at RT overnight. The resulting yellow solution was partitioned between EtOAc-THF (1:1) and brine. The layers were separated and the aqueous layer was extracted with EtOAc (3×). The combined organic layers were washed with brine, dried over anhydrous magnesium sulfate and concentrated in vacuo to afford the title compound as orange solid (381 mg, 98%). MS (ESI): 378.2 [M+H]⁺

### Step b) tert-butyl N-[(3R)-7-(5-bromo-1,3,4-oxadiazol-2-yl)-4-oxo-3,5-dihydro-2H-1,5-benzothiazepin-3-yl]carbamate

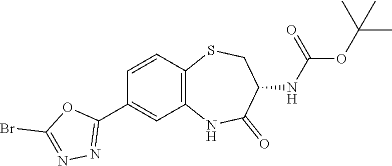 [SMILES: CC(C)(C)OC(=O)N[C@H]1CSc2ccc(-c3nnc(Br)o3)cc2NC1=O]

A solution of tert-butyl N-[(3R)-7-(5-amino-1,3,4-oxadiazol-2-yl)-4-oxo-3,5-dihydro-2H-1,5-benzothiazepin-3-yl]carbamate (381 mg, 1.01 mmol, 1.0 eq) in acetonitrile (5 mL) was degassed with argon for 5 min. Then copper (II) bromide (338 mg, 1.51 mmol, 1.5 eq) and isoamyl nitrite (141.9 mg, 163.12 uL, 1.21 mmol, 1.2 eq) were added at RT to form a dark green suspension. The reaction mixture was stirred for 1 h. The reaction mixture was diluted with EtOAc (100 ml) and 1N HCl (100 ml). The phases were separated and the aqueous phase was extracted with EtOAc (2×150 ml). The combined organic phases were dried over anhydrous sodium sulfate, filtered and concentrated in vacuo. The remaining solid was purified by column chromatography on silica gel (0-100% EtOAc in heptane) to afford the title compound (102 mg, 23%) as orange solid. MS (ESI): 439.1 [M−H]

### Step c) tert-butyl N-[(3R)-7-(5-bromo-1,3,4-oxadiazol-2-yl)-1,1,4-trioxo-3,5-dihydro-2H-1λ⁶,5-benzothiazepin-3-yl]carbamate

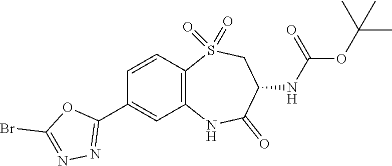 [SMILES: CC(C)(C)OC(=O)N[C@H]1CS(=O)(=O)c2ccc(-c3nnc(Br)o3)cc2NC1=O]

The title compound was prepared from tert-butyl N-[(3R)-7-(5-bromo-1,3,4-oxadiazol-2-yl)-4-oxo-3,5-dihydro-2H-1,5-benzothiazepin-3-yl]carbamate (905 mg, 2.05 mmol) in analogy to general procedure 10 and was obtained as white solid (524 mg, 53%). MS (ESI): 417.0 [M-isobutene+H]⁺

### Step d) tert-butyl N-[(3R)-7-[5-[(3,3-difluoro-1-methyl-cyclobutyl)amino]-1,3,4-oxadiazol-2-yl]-1,1,4-trioxo-3,5-dihydro-2H-1λ⁶, 5-benzothiazepin-3-yl]carbamate

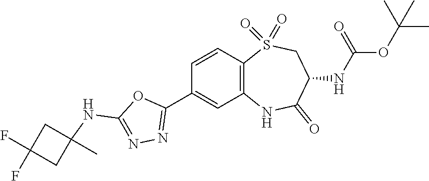 [SMILES: CC1(Nc2nnc(-c3ccc4c(c3)NC(=O)[C@@H](NC(=O)OC(C)(C)C)CS4(=O)=O)o2)CC(F)(F)C1]

The title compound was prepared from tert-butyl N-[(3R)-7-(5-bromo-1,3,4-oxadiazol-2-yl)-1,1,4-trioxo-3,5-dihydro-2H-1λ⁶,5-benzothiazepin-3-yl]carbamate (25 mg, 0.052 mmol, 1.0 eq) and (3,3-difluoro-1-methyl-cyclobutyl)amine hydrochloride (9.8 mg, 0.061 mmol, 1.2 eq) in analogy to general procedure 13 and was obtained as yellow amorphous solid (30.5 mg, 43%). MS (ESI): 514.2 [M+H]⁺

### Step e) tert-butyl N-[(3R)-7-[5-[(3,3-difluoro-1-methyl-cyclobutyl)amino]-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate

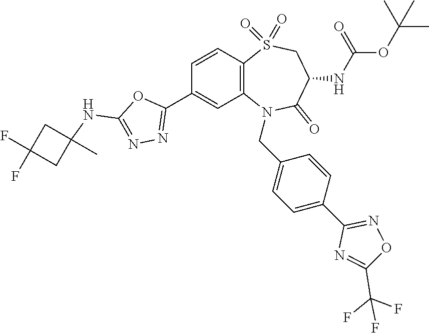 [SMILES: CC1(Nc2nnc(-c3ccc4c(c3)N(Cc3ccc(-c5noc(C(F)(F)F)n5)cc3)C(=O)[C@@H](NC(=O)OC(C)(C)C)CS4(=O)=O)o2)CC(F)(F)C1]

The title compound was prepared from tert-butyl N-[(3R)-7-[5-[(3,3-difluoro-1-methyl-cyclobutyl)amino]-1,3,4-oxadiazol-2-yl]-1,1,4-trioxo-3,5-dihydro-2H-1λ⁶,5-benzothiazepin-3-yl]carbamate (30.5 mg, 0.023 mmol, 1.0 eq) and 3-[4-(bromomethyl)phenyl]-5-(trifluoromethyl)-1,2,4-oxadiazole (10.1 mg, 0.033 mmol, 1.5 eq, CAS 2093101-98-3) in analogy to general procedure 1a and was obtained as white solid (5.7 mg, 32%). MS (ESI): 740.2 [M+H]⁺

### Step f) (3R)-3-amino-7-[5-[(3,3-difluoro-1-methyl-cyclobutyl)amino]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

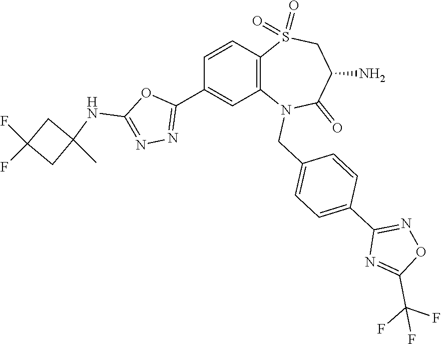 [SMILES: CC1(Nc2nnc(-c3ccc4c(c3)N(Cc3ccc(-c5noc(C(F)(F)F)n5)cc3)C(=O)[C@@H](N)CS4(=O)=O)o2)CC(F)(F)C1]

The title compound was prepared from tert-butyl N-[(3R)-7-[5-[(3,3-difluoro-1-methyl-cyclobutyl)amino]-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (5.7 mg, 0.007 mmol) in analogy to general procedure 11a and was obtained as white solid (3.2 mg, 66%). MS (ESI): 640.3 [M+H]⁺

The examples of the following table were prepared in analogy to Example 72, using the appropriate amine and benzyl halide building block with the indicated general procedure.

| | | | | MS, |
|---|---|---|---|---|
| | | | Building Block | ESI: |
| Ex. | Structure | Systematic Name | (synthesis step) | m/z |
| 73 | 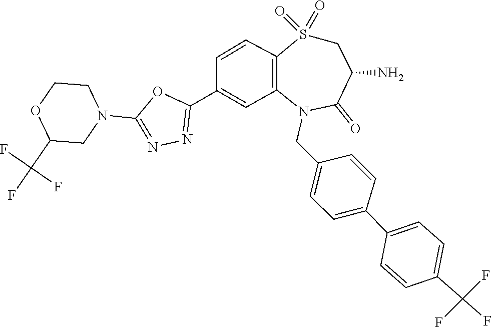 [SMILES: N[C@H]1CS(=O)(=O)c2ccc(-c3nnc(N4CCOC(C(F)(F)F)C4)o3)cc2N(Cc2ccc(-c3ccc(C(F)(F)F)cc3)cc2)C1=O] | (3R)-3-amino-1,1- dioxo-7-[5-[2- (trifluoromethyl) morpholin-4-yl]-1,3,4- oxadiazol-2-yl]-5-[[4- [4- (trifluoromethyl)phenyl] phenyl]methyl]-2,3- dihydro-1λ⁶,5- benzothiazepin-4-one (*) | 2- (trifluoromethyl) morpholine (CAS 1196532-95-2, step d); 4- (bromomethyl)- 4′- (trifluoromethyl)- 1,1′-biphenyl (CAS 613241-14- 8, step e) | 682.5 [M + H]⁺ |
| 74 | 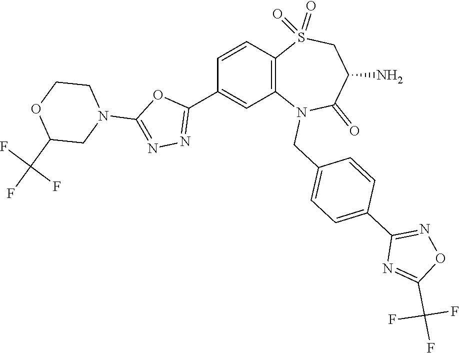 [SMILES: N[C@H]1CS(=O)(=O)c2ccc(-c3nnc(N4CCOC(C(F)(F)F)C4)o3)cc2N(Cc2ccc(-c3noc(C(F)(F)F)n3)cc2)C1=O] | (3R)-3-amino-1,1- dioxo-7-[5-[2- (trifluoromethyl) morpholin-4-yl]-1,3,4- oxadiazol-2-yl]-5-[[4- [5-(trifluoromethyl)- 1,2,4-oxadiazol-3- yl]phenyl]methyl]-2,3- dihydro-1λ⁶,5- benzothiazepin-4-one (*) | 2- (trifluoromethyl) morpholine (CAS 1196532-95-2, step d) | 674.4 [M + H]⁺ |
| 75 | 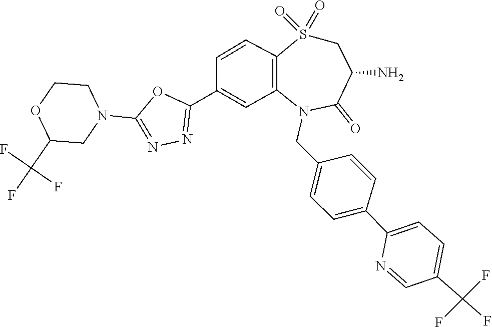 [SMILES: N[C@H]1CS(=O)(=O)c2ccc(-c3nnc(N4CCOC(C(F)(F)F)C4)o3)cc2N(Cc2ccc(-c3ccc(C(F)(F)F)cn3)cc2)C1=O] | (3R)-3-amino-1,1- dioxo-7-[5-[2- (trifluoromethyl) morpholin-4-yl]-1,3,4- oxadiazol-2-yl]-5-[[4- [5-(trifluoromethyl)-2- pyridyl]phenyl]methyl]- 2,3-dihydro-1λ⁶,5- benzothiazepin-4-one (*) | 2- (trifluoromethyl) morpholine (CAS 1196532-95-2, step d); 2-[4- (bromomethyl) phenyl]-5- (trifluoromethyl) pyridine (CAS 1056641-21-4, step e) | 683.4 [M + H]⁺ |
| 76 | 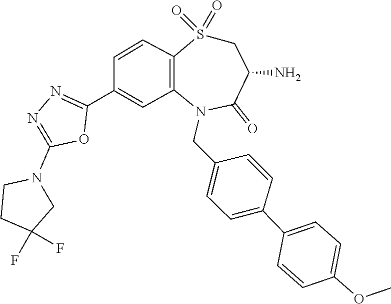 [SMILES: COc1ccc(-c2ccc(CN3C(=O)[C@@H](N)CS(=O)(=O)c4ccc(-c5nnc(N6CCC(F)(F)C6)o5)cc43)cc2)cc1] | (3R)-3-amino-7-[5-(3,3- difluoropyrrolidin-1-yl)- 1,3,4-oxadiazol-2-yl]-5- [[4-(4- methoxyphenyl)phenyl] methyl]-1,1-dioxo-2,3- dihydro-1λ⁶,5- benzothiazepin-4-one (*) | 3,3- difluoropyrro- lidine (CAS 316131- 01-8, step d); 4- (bromomethyl)- 4′-methoxy-1,1′- biphenyl CAS, 20854-61-9, step e) | 596.3 [M + H]⁺ |
| 77 | 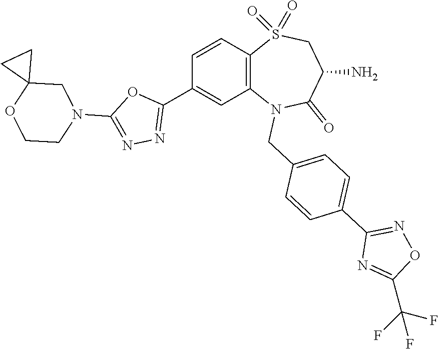 [SMILES: N[C@H]1CS(=O)(=O)c2ccc(-c3nnc(N4CCOC5(CC5)C4)o3)cc2N(Cc2ccc(-c3noc(C(F)(F)F)n3)cc2)C1=O] | (3R)-3-amino-7-[5-(4- oxa-7- azaspiro[2.5]octan-7- yl)-1,3,4-oxadiazol-2- yl]-1,1-dioxo-5-[4-[5- (trifluoromethyl)-1,2,4- oxadiazol-3- yl]phenyl]methyl]-2,3- dihydro-1λ⁶,5- benzothiazepin-4-one (*) | 4-oxa-7- azaspiro[2.5]octane hydrochloride (CAS 1427195- 23-0, step d) | 632.5 [M + H]⁺ |
| 78 | 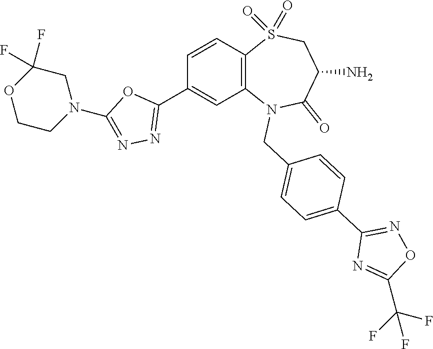 [SMILES: N[C@H]1CS(=O)(=O)c2ccc(-c3nnc(N4CCOC(F)(F)C4)o3)cc2N(Cc2ccc(-c3noc(C(F)(F)F)n3)cc2)C1=O] | (3R)-3-amino-7-[5-(2,2- difluoromorpholin-4- yl)-1,3,4-oxadiazol-2- yl]-1,1-dioxo-5-[[4-[5- (trifluoromethyl)-1,2,4- oxadiazol-3- yl]phenyl]methyl]-2,3- dihydro-1λ⁶,5- benzothiazepin-4-one (*) | 2,2- difluoromorpholine (CAS 1263180-85-3, step d) | 642.3 [M + H]⁺ |
| 79 | 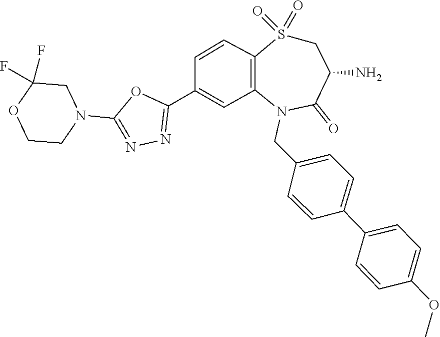 [SMILES: COc1ccc(-c2ccc(CN3C(=O)[C@@H](N)CS(=O)(=O)c4ccc(-c5nnc(N6CCOC(F)(F)C6)o5)cc43)cc2)cc1] | (3R)-3-amino-7-[5-(2,2- difluoromorpholin-4- yl)-1,3,4-oxadiazol-2- yl]-5-[[4-(4- methoxyphenyl)phenyl] methyl]-1,1-dioxo-2,3- dihydro-1λ⁶,5- benzothiazepin-4-one (*) | 2,2- difluoromorpholine (CAS 1263180-85-3, step d); 4- (bromomethyl)- 4′-methoxy-1,1′- biphenyl CAS, 20854-61-9, step e) | 612.4 [M + H]⁺ |
| 80 | 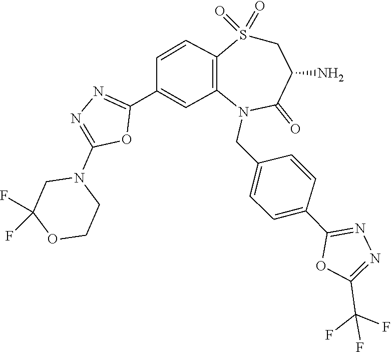 [SMILES: N[C@H]1CS(=O)(=O)c2ccc(-c3nnc(N4CCOC(F)(F)C4)o3)cc2N(Cc2ccc(-c3nnc(C(F)(F)F)o3)cc2)C1=O] | (3R)-3-amino-7-[5-(2,2- difluoromorpholin-4- yl)-1,3,4-oxadiazol-2- yl]-1,1-dioxo-5-[[4-[5- (trifluoromethyl)-1,3,4- oxadiazol-2- yl]phenyl]methyl]-2,3- dihydro-1λ⁶,5- benzothiazepin-4-one (*) | 2,2- difluoromorpholine (CAS 1263180-85-3, step d); Intermediate 4 (step e) | 642.1 [M + H]⁺ |
| 81 | 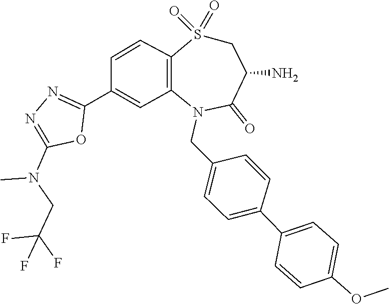 | (3R)-3-amino-5-[[4-(4- methoxyphenyl)phenyl] methyl]-7-[5- [methyl(2,2,2- trifluoroethyl)amino]- 1,3,4-oxadiazol-2-yl]- 1,1-dioxo-2,3-dihydro- 1λ⁶,5-benzothiazepin-4- one (*) | 2,2,2-Trifluoro- N- methylethanamine (CAS 2730-67- 8, step d); 4- (chloromethyl)- 4′-methoxy-1,1′- biphenyl (step e) | 602.3 [M + H]⁺ |
| 82 | 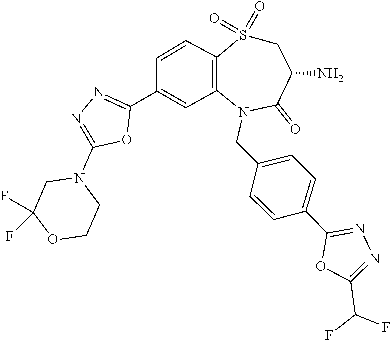 [SMILES: N[C@H]1CS(=O)(=O)c2ccc(-c3nnc(N4CCOC(F)(F)C4)o3)cc2N(Cc2ccc(-c3nnc(C(F)F)o3)cc2)C1=O] | (3R)-3-amino-5-[[4-[5- (difluoromethyl)-1,3,4- oxadiazol-2- yl]phenyl]methyl]-7-[5- (2,2-difluoromorpholin- 4-yl)-1,3,4-oxadiazol-2- yl]-1,1-dioxo-2,3- dihydro-1λ⁶,5- benzothiazepin-4-one (*) | 2,2- difluoromorpholine (CAS 1263180-85-3, step d); Intermediate 17 (step e) | 624.2 [M + H]⁺ |

(*) as hydrochloride salt

### Example 83

### (3R)-3-amino-7-(5-tert-butyl-1,3,4-oxadiazol-2-yl)-5-[[4-(6-methoxy-3-pyridyl)phenyl]methyl]-1,1-dioxo-2,3-dihydro-1λ⁶,5-benzothiazepin-4-one

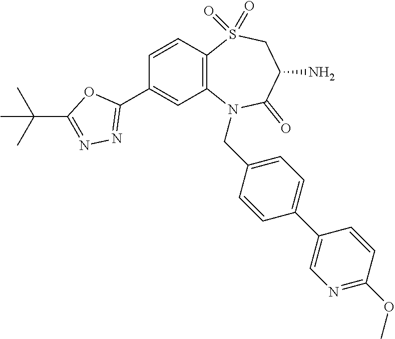 [SMILES: COc1ccc(-c2ccc(CN3C(=O)[C@@H](N)CS(=O)(=O)c4ccc(-c5nnc(C(C)(C)C)o5)cc43)cc2)cn1]

### Step a) methyl (3R)-3-(benzyloxycarbonylamino)-4-oxo-3,5-dihydro-2H-1,5-benzothiazepine-7-carboxylate

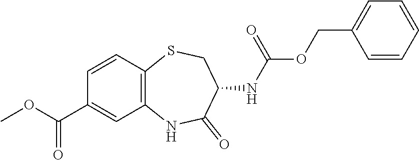 [SMILES: COC(=O)c1ccc2c(c1)NC(=O)[C@@H](NC(=O)OCc1ccccc1)CS2]

To a solution of methyl (3R)-3-(tert-butoxycarbonylamino)-4-oxo-3,5-dihydro-2H-1,5-benzothiazepine-7-carboxylate (300 mg, 0.851 mmol, 1 eq, CAS 2089150-62-7) in 1,4-dioxane (1.5 mL) was added 4 M HCl in dioxane (2.36 g, 1.97 mL, 7.88 mmol, 9.2 eq) at RT and stirred overnight. The reaction mixture was diluted with EtOAc (40 ml) and 0.5 M NaOH (40 ml). The layers were separated and the aqueous layer extracted with two 40 ml portions of EtOAc. The combined organic layers were washed with one 40 ml portion of brine, dried over anhydrous sodium sulfate and concentrated in vacuo to afford a crude yellow solid (141.7 mg). The crude solid was suspended in DCM (3 mL). DIEA (165 mg, 223 μL, 1.28 mmol, 1.5 eq) and benzyl chloroformate (95.3 mg, 79.8 uL, 0.56 mmol, 0.65 eq) were added at RT to give a dark yellow solution which was was stirred for 2 h. The reaction mixture was concentrated and purified by column chromatography on silica gel (0-40% EtOAc in heptane) to afford methyl (3R)-3-(benzyloxycarbonylamino)-4-oxo-3,5-dihydro-2H-1,5-benzothiazepine-7-carboxylate (196 mg, 59%) as white solid. MS (ESI): 387.2 [M+H]⁺

### Step b) (3R)-3-(benzyloxycarbonylamino)-4-oxo-3,5-dihydro-2H-1,5-benzothiazepine-7-carboxylic acid

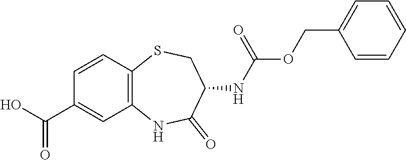 [SMILES: O=C(N[C@H]1CSc2ccc(C(=O)O)cc2NC1=O)OCc1ccccc1]

The title compound was prepared from methyl (3R)-3-(benzyloxycarbonylamino)-4-oxo-3,5-dihydro-2H-1,5-benzothiazepine-7-carboxylate (320 mg, 0.82 mmol) in analogy to general procedure 2 and was obtained as yellow solid (364 mg, 99%). MS (ESI): 373.1 [M+H]⁺

### Step c) benzyl N-[(3R)-7-[(2,2-dimethylpropanoylamino)carbamoyl]-4-oxo-3,5-dihydro-2H-1,5-benzothiazepin-3-yl]carbamate

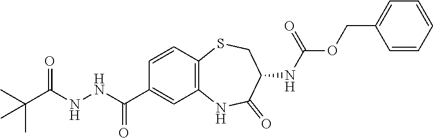 [SMILES: CC(C)(C)C(=O)NNC(=O)c1ccc2c(c1)NC(=O)[C@@H](NC(=O)OCc1ccccc1)CS2]

The title compound was prepared from (3R)-3-(benzyloxycarbonylamino)-4-oxo-3,5-dihydro-2H-1,5-benzothiazepine-7-carboxylic acid (364 mg, 0.81 mmol, 1 eq) and 2,2-dimethylpropionohydrazide (131.93 mg, 1.14 mmol, 1.4 eq) in analogy to general procedure 4a and was obtained as light yellow solid (687 mg, 99%). MS (ESI): 471.2 [M+H]⁺

### Step d) benzyl N-[(3R)-7-(5-tert-butyl-1,3,4-oxadiazol-2-yl)-1,1,4-trioxo-3,5-dihydro-2H-1λ⁶,5-benzothiazepin-3-yl]carbamate

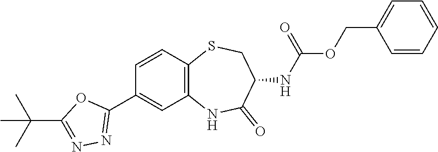 [SMILES: CC(C)(C)c1nnc(-c2ccc3c(c2)NC(=O)[C@@H](NC(=O)OCc2ccccc2)CS3)o1]

The title compound was prepared from benzyl N-[(3R)-7-[(2,2-dimethylpropanoylamino)carbamoyl]-4-oxo-3,5-dihydro-2H-1,5-benzothiazepin-3-yl]carbamate (687 mg, 0.803 mmol) in analogy to general procedure 5a and was obtained as yellow oil (321 mg, 85%). MS (ESI): 453.2 [M+H]⁺

### Step e)-N-[(3R)-7-(5-tert-butyl-1,3,4-oxadiazol-2-yl)-1,1,4-triketo-3,5-dihydro-2H-1?6,5-benzothiazepin-3-yl]carbamic acid benzyl ester

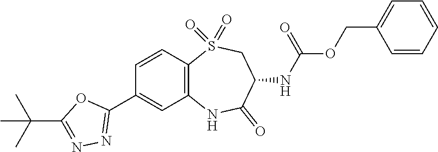 [SMILES: CC(C)(C)c1nnc(-c2ccc3c(c2)NC(=O)[C@@H](NC(=O)OCc2ccccc2)CS3(=O)=O)o1]

The title compound was prepared from benzyl N-[(3R)-7-(5-tert-butyl-1,3,4-oxadiazol-2-yl)-1,1,4-trioxo-3,5-dihydro-2H-1λ⁶,5-benzothiazepin-3-yl]carbamate (321 mg, 0.65 mmol) in analogy to general procedure 10 and was obtained as white solid (182 mg, 57%). MS (ESI): 485.2 [M+H]⁺

### Step f) benzyl N-[(3R)-7-(5-tert-butyl-1,3,4-oxadiazol-2-yl)-5-[[4-(6-methoxy-3-pyridyl)phenyl]methyl]-1,1,4-trioxo-2,3-dihydro-1λ⁶,5-benzothiazepin-3-yl]carbamate

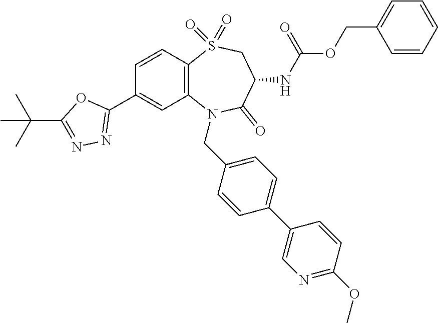 [SMILES: COc1ccc(-c2ccc(CN3C(=O)[C@@H](NC(=O)OCc4ccccc4)CS(=O)(=O)c4ccc(-c5nnc(C(C)(C)C)o5)cc43)cc2)cn1]

The title compound was prepared from N-[(3R)-7-(5-tert-butyl-1,3,4-oxadiazol-2-yl)-1,1,4-triketo-3,5-dihydro-2H-1?6,5-benzothiazepin-3-yl]carbamic acid benzyl ester (55 mg, 0.114 mmol, 1 equiv) and 5-[4-(Bromomethyl)phenyl]-2-methoxypyridine (47.36 mg, 0.17 mmol, 1.5 eq, CAS 234109-32-1) in analogy to general procedure 1a and was obtained as white solid (48 mg, 31%). MS (ESI): 682.5 [M+H]⁺

### Step g) (3R)-3-amino-7-(5-tert-butyl-1,3,4-oxadiazol-2-yl)-5-[[4-(6-methoxy-3-pyridyl)phenyl]methyl]-1,1-dioxo-2,3-dihydro-1λ⁶,5-benzothiazepin-4-one

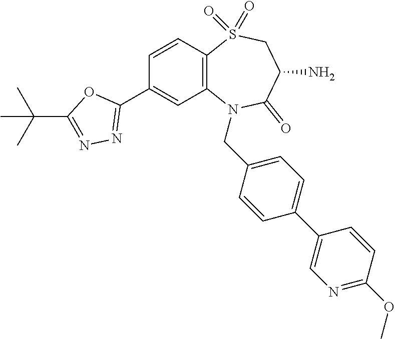 [SMILES: COc1ccc(-c2ccc(CN3C(=O)[C@@H](N)CS(=O)(=O)c4ccc(-c5nnc(C(C)(C)C)o5)cc43)cc2)cn1]

To a solution of benzyl N-[(3R)-7-(5-tert-butyl-1,3,4-oxadiazol-2-yl)-5-[[4-(6-methoxy-3-pyridyl)phenyl]methyl]-1,1,4-trioxo-2,3-dihydro-1λ⁶,5-benzothiazepin-3-yl]carbamate (48 mg, 0.035 mmol, 1 eq) in MeOH (1.76 mL) and THF (1.76 mL) under Ar atmosphere was added Pd/C (0.38 mg, 0.004 mmol, 0.1 eq) and the mixture was stirred under an atmosphere of hydrogen overnight. The mixture was filtered through a plug of celite, which was washed with MeOH and THF. The filtrate was concentrated and the remaining crude was purified using prep-HPLC to afford (3R)-3-amino-7-(5-tert-butyl-1,3,4-oxadiazol-2-yl)-1,1-diketo-5-[4-(6-methoxy-3-pyridyl)benzyl]-2,3-dihydro-1λ⁶,5-benzothiazepin-4-one (5.8 mg, 27%) as white solid. MS (ESI): 548.3 [M+H]⁺

### Example 165

### (3R)-3-amino-1,1-dioxo-7-[5-(2,2,2-trifluoro-1,1-dimethyl-ethyl)-1,3,4-oxadiazol-2-yl]-5-[[4-[5-(trifluoromethoxy)-2-pyridyl]phenyl]methyl]-2,3-dihydro-1λ⁶,5-benzothiazepin-4-one

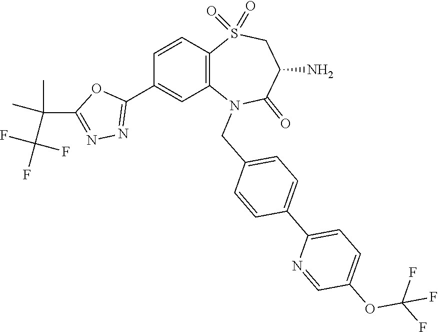 [SMILES: CC(C)(c1nnc(-c2ccc3c(c2)N(Cc2ccc(-c4ccc(OC(F)(F)F)cn4)cc2)C(=O)[C@@H](N)CS3(=O)=O)o1)C(F)(F)F]

### Step a) tert-butyl N-[(3R)-4-oxo-7-[5-(2,2,2-trifluoro-1,1-dimethyl-ethyl)-1,3,4-oxadiazol-2-yl]-3,5-dihydro-2H-1λ⁶,5-benzothiazepin-3-yl]carbamate

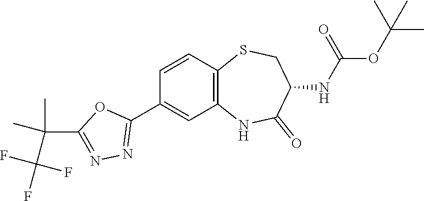 [SMILES: CC(C)(C)OC(=O)N[C@H]1CSc2ccc(-c3nnc(C(C)(C)C(F)(F)F)o3)cc2NC1=O]

3,3,3-trifluoro-2,2-dimethyl-propionic acid (132.88 mg, 851.3 μmol, 1.5 eq, CAS 889940-13-0) and tert-butyl N-[(3R)-7-(hydrazinecarbonyl)-4-oxo-3,5-dihydro-2H-1,5-benzothiazepin-3-yl]carbamate (200 mg, 567.52 μmol, 1.0 eq, Example 19, step b) were suspended in THF (5 mL). To this suspension was added DIPEA (220 mg, 297.4 uL, 1.7 mmol, 3.0 eq) to give a clear solution. Finally, HATU (323.7 mg, 851.3 μmol, 1.5 eq) was and the reaction mixture stirred for 2 h. The reaction was diluted with water and extracted with EtOAc (3×). The combined organic phases were dried over sodium sulfate, filtered, and concentrated under reduced pressure. The remaining crude material was suspended in acetonitrile (5 mL), DIPEA (146.7 mg, 198 μL, 1.14 mmol, 2.0 eq) and p-TsCl (324.6 mg, 1.7 mmol, 3.0 eq) were added. The resulting solution was stirred at room temperature for 90 min.

The reaction mixture was partitioned between EtOAc and 1 M NaOH. The layers were separated and the aqueous layer was extracted with EtOAc (2×). The combined organic layers were washed with brine, dried over sodium sulfate, filtered and concentrated under reduced pressure. The remaining residue was purified by column chromatography on silica gel (0-35% EtOAc in heptanes) to afford the title compound (207.9 mg, 74%) as white crystalline solid. MS (ESI): 471.2 [M+H]⁺

### Step b) tert-butyl N-[(3R)-1,1,4-trioxo-7-[5-(2,2,2-trifluoro-1,1-dimethyl-ethyl)-1,3,4-oxadiazol-2-yl]-3,5-dihydro-2H-1λ⁶, 5-benzothiazepin-3-yl]carbamate

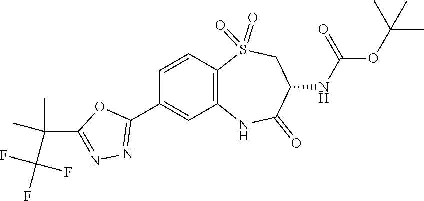 [SMILES: CC(C)(C)OC(=O)N[C@H]1CS(=O)(=O)c2ccc(-c3nnc(C(C)(C)C(F)(F)F)o3)cc2NC1=O]

The title compound as prepared from tert-butyl N-[(3R)-4-oxo-7-[5-(2,2,2-trifluoro-1,1-dimethyl-ethyl)-1,3,4-oxadiazol-2-yl]-3,5-dihydro-2H-1,5-benzothiazepin-3-yl]carbamate (210 mg, 0.44 mmol, 1.0 eq) in analogy to general procedure 10 and was obtained as a light yellow solid (230 mg, 0.46 mmol, 93% yield). MS (ESI): 449.1 [M+H]⁺

### Step c) tert-butyl N-[(3R)-1,1,4-trioxo-7-[5-(2,2,2-trifluoro-1,1-dimethyl-ethyl)-1,3,4-oxadiazol-2-yl]-5-[[4-[5-(trifluoromethoxy)-2-pyridyl]phenyl]methyl]-2,3-dihydro-1λ⁶, 5-benzothiazepin-3-yl]carbamate

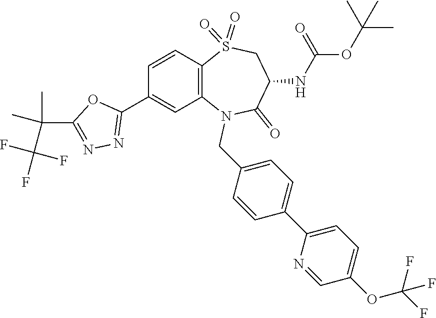 [SMILES: CC(C)(C)OC(=O)N[C@H]1CS(=O)(=O)c2ccc(-c3nnc(C(C)(C)C(F)(F)F)o3)cc2N(Cc2ccc(-c3ccc(OC(F)(F)F)cn3)cc2)C1=O]

The title compound was prepared from tert-butyl N-[(3R)-1,1,4-trioxo-7-[5-(2,2,2-trifluoro-1,1-dimethyl-ethyl)-1,3,4-oxadiazol-2-yl]-3,5-dihydro-2H-1λ⁶,5-benzothiazepin-3-yl]carbamate (100 mg, 0.2 mmol, 1.0 eq) in analogy to general procedure 1a using Intermediate 38 (13.5 mg, 0.04 mmol, 1.05 eq) and was obtained as light yellow oil (100 mg, 0.13 mmol, 65%). MS (ESI): 756.3 [M+H]⁺

### Step d) (3R)-3-amino-1,1-dioxo-7-[5-(2,2,2-trifluoro-1,1-dimethyl-ethyl)-1,3,4-oxadiazol-2-yl]-5-[[4-[5-(trifluoromethoxy)-2-pyridyl]phenyl]methyl]-2,3-dihydro-1λ⁶, 5-benzothiazepin-4-one

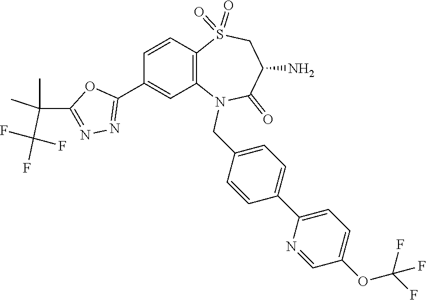 [SMILES: CC(C)(c1nnc(-c2ccc3c(c2)N(Cc2ccc(-c4ccc(OC(F)(F)F)cn4)cc2)C(=O)[C@@H](N)CS3(=O)=O)o1)C(F)(F)F]

The title compound was prepared from tert-butyl N-[(3R)-1,1,4-trioxo-7-[5-(2,2,2-trifluoro-1,1-dimethyl-ethyl)-1,3,4-oxadiazol-2-yl]-5-[[4-[5-(trifluoromethoxy)-2-pyridyl]phenyl]methyl]-2,3-dihydro-1λ⁶,5-benzothiazepin-3-yl]carbamate (98 mg, 0.13 mmol) in analogy to general procedure 11c and was obtained as light yellow solid (76.8 mg, 0.11 mmol, 83% yield). MS (ESI): 656.1 [M+H]⁺

The examples of the following table were prepared in analogy to Example 165, using the appropriate benzyl bromide building block.

| | | | | MS, |
|---|---|---|---|---|
| | | | | ESI: |
| Ex. | Structure | Systematic Name | Building Block | m/z |
| 166 | 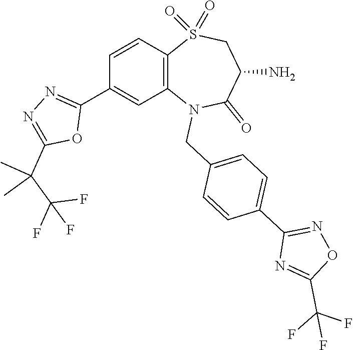 [SMILES: CC(C)(c1nnc(-c2ccc3c(c2)N(Cc2ccc(-c4noc(C(F)(F)F)n4)cc2)C(=O)[C@@H](N)CS3(=O)=O)o1)C(F)(F)F] | (3R)-3-amino-1,1- dioxo-7-[5-(2,2,2- trifluoro-1,1- dimethyl-ethyl)- 1,3,4-oxadiazol-2- yl]-5-[[4-[5- (trifluoromethyl)- 1,2,4-oxadiazol-3- yl]phenyl]methyl]- 2,3-dihydro-1λ⁶,5- benzothiazepin-4- one (*) | 3-[4- (bromomethyl) phenyl]- 5- (trifluoromethyl)- 1,2,4-oxadiazole (CAS 2093101-98-3) | 631.3 [M + H]⁺ |
| 167 | 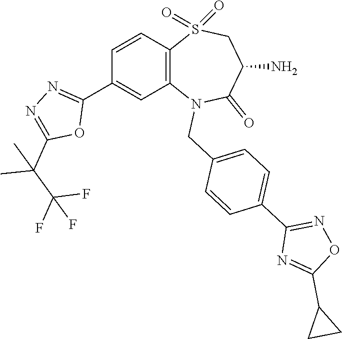 [SMILES: CC(C)(c1nnc(-c2ccc3c(c2)N(Cc2ccc(-c4noc(C5CC5)n4)cc2)C(=O)[C@@H](N)CS3(=O)=O)o1)C(F)(F)F] | (3R)-3-amino-5- [[4-(5- cyclopropyl-1,2,4- oxadiazol-3- yl)phenyl]methyl]- 1,1-dioxo-7-[5- (2,2,2-trifluoro- 1,1-dimethyl- ethyl)-1,3,4- oxadiazol-2-yl]- 2,3-dihydro-1λ⁶,5- benzothiazepin-4- one (*) | Intermediate 16 | 603.3 [M + H]⁺ |

(*) as hydrochloride salt

### Example 168

### (3R)-3-amino-5-[[4-(5-cyclopropyl-1,2,4-oxadiazol-3-yl)phenyl]methyl]-1,1-dioxo-7-[5-(1,2,2,2-tetrafluoroethyl)-1,3,4-oxadiazol-2-yl]-2,3-dihydro-1λ⁶,5-benzothiazepin-4-one

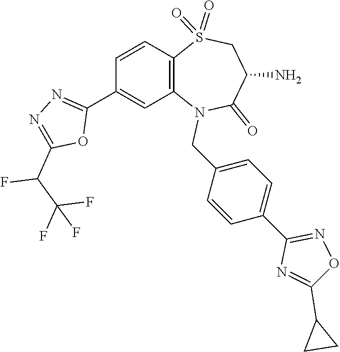 [SMILES: N[C@H]1CS(=O)(=O)c2ccc(-c3nnc(C(F)C(F)(F)F)o3)cc2N(Cc2ccc(-c3noc(C4CC4)n3)cc2)C1=O]

### Step a) Methyl (3R)-3-(tert-butoxycarbonylamino)-5-[[4-(5-cyclopropyl-1,2,4-oxadiazol-3-yl)phenyl]methyl]-4-oxo-2,3-dihydro-1,5-benzothiazepine-7-carboxylate

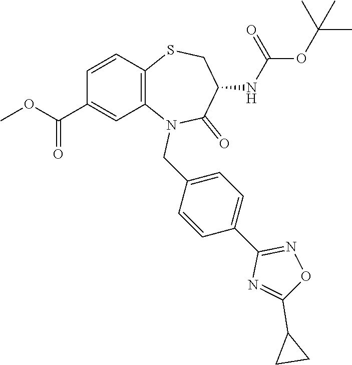 [SMILES: COC(=O)c1ccc2c(c1)N(Cc1ccc(-c3noc(C4CC4)n3)cc1)C(=O)[C@@H](NC(=O)OC(C)(C)C)CS2]

The title compound was prepared from methyl (3R)-3-(tert-butoxycarbonylamino)-4-oxo-3,5-dihydro-2H-1,5-benzothiazepine-7-carboxylate (939.3 mg, 2.67 mmol, 1.0 eq, CAS 2089150-62-7) in analogy to general procedure 1a using Intermediate 16 (800 mg, 2.67 mmol, 1.0 eq) and was obtained as light yellow solid (1.88 g, 99%). MS (ESI): 451.3 [M+H-Boc]⁺

### Step b) (3R)-3-(tert-butoxycarbonylamino)-5-[[4-(5-cyclopropyl-1,2,4-oxadiazol-3-yl)phenyl]methyl]-4-oxo-2,3-dihydro-1,5-benzothiazepine-7-carboxylic acid

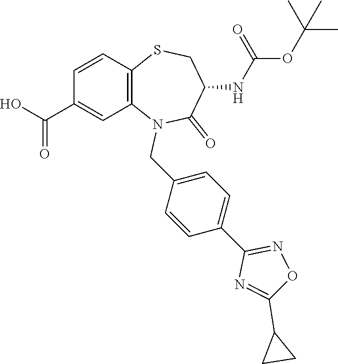 [SMILES: CC(C)(C)OC(=O)N[C@H]1CSc2ccc(C(=O)O)cc2N(Cc2ccc(-c3noc(C4CC4)n3)cc2)C1=O]

The title compound was prepared from methyl (3R)-3-(tert-butoxycarbonylamino)-5-[[4-(5-cyclopropyl-1,2,4-oxadiazol-3-yl)phenyl]methyl]-4-oxo-2,3-dihydro-1,5-benzothiazepine-7-carboxylate (1.04 g, 1.88 mmol) in analogy to general procedure 2 and was obtained as light yellow foam (1.62 g, 99%). MS (ESI): 437.2 [M+H-Boc]⁺

### Step c) tert-butyl N-[(3R)-5-[[4-(5-cyclopropyl-1,2,4-oxadiazol-3-yl)phenyl]methyl]-7-(hydrazinecarbonyl)-4-oxo-2,3-dihydro-1,5-benzothiazepin-3-yl]carbamate

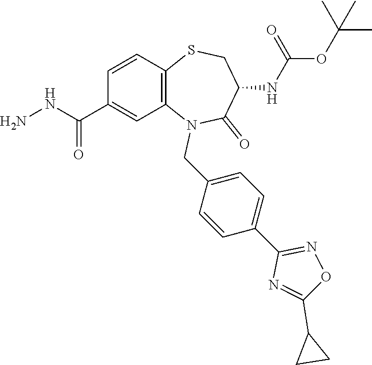 [SMILES: CC(C)(C)OC(=O)N[C@H]1CSc2ccc(C(=O)NN)cc2N(Cc2ccc(-c3noc(C4CC4)n3)cc2)C1=O]

The title compound was prepared from (3R)-3-(tert-butoxycarbonylamino)-5-[[4-(5-cyclopropyl-1,2,4-oxadiazol-3-yl)phenyl]methyl]-4-oxo-2,3-dihydro-1,5-benzothiazepine-7-carboxylic acid (1.62 g, 3.01 mmol) in analogy to general procedure 3 and was obtained as light yellow viscous oil (0.997 g, 60%). MS (ESI): 495.2 [M+H-isobutene]⁺

### Step d) tert-butyl N-[(3R)-5-[[4-(5-cyclopropyl-1,2,4-oxadiazol-3-yl)phenyl]methyl]-4-oxo-7-[5-(1,2,2,2-tetrafluoroethyl)-1,3,4-oxadiazol-2-yl]-2,3-dihydro-1,5-benzothiazepin-3-yl]carbamate

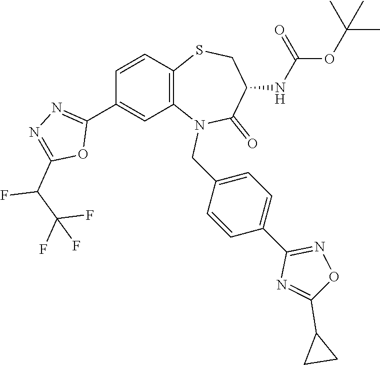 [SMILES: CC(C)(C)OC(=O)N[C@H]1CSc2ccc(-c3nnc(C(F)C(F)(F)F)o3)cc2N(Cc2ccc(-c3noc(C4CC4)n3)cc2)C1=O]

To a solution of tert-butyl N-[(3R)-5-[[4-(5-cyclopropyl-1,2,4-oxadiazol-3-yl)phenyl]methyl]-7-(hydrazinecarbonyl)-4-oxo-2,3-dihydro-1,5-benzothiazepin-3-yl]carbamate (80 mg, 145.3 μmol, 1.0 eq) in THF (969 uL) was added 2,3,3,3-tetrafluoropropionic acid (42.4 mg, 290.6 μmol, 2.0 eq, CAS 359-49-9)), DIPEA (37.6 mg, 50.8 uL, 290.6 μmol, 2.0 eq) and HATU (82.9 mg, 217.9 μmol, 1.5 eq). The resulting solution was stirred at room temperature for 2 h. The reaction was directly concentrated under reduced pressure until all volatiles were removed. The remaining residue was dissolved in THF (969 uL) and Burgess reagent (173 mg, 726 μmol, 5.0 eq) was added in one portion at room temperature and stirred for 2 h. The reaction was diluted with water and extracted with EtOAc (3×). The combined organic extracts were dried over sodium sulfate, filtered and concentrated under reduced pressure. The remaining crude material was purified by column chromatography on silica gel (0-50% EtOAc in heptanes) to afford the title compound (27 mg, 29%) as white solid. MS (ESI): 605.2 [M+H-isobutene]⁺

### Step e) tert-butyl N-[(3R)-5-[[4-(5-cyclopropyl-1,2,4-oxadiazol-3-yl)phenyl]methyl]-1,1,4-trioxo-7-[5-(1,2,2,2-tetrafluoroethyl)-1,3,4-oxadiazol-2-yl]-2,3-dihydro-1λ⁶, 5-benzothiazepin-3-yl]carbamate

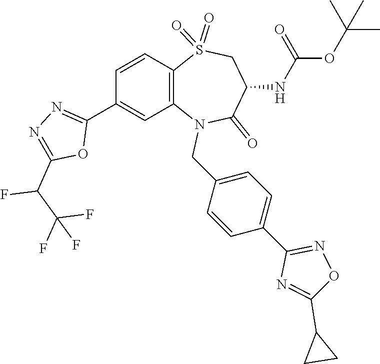 [SMILES: CC(C)(C)OC(=O)N[C@H]1CS(=O)(=O)c2ccc(-c3nnc(C(F)C(F)(F)F)o3)cc2N(Cc2ccc(-c3noc(C4CC4)n3)cc2)C1=O]

The title compound was prepared from tert-butyl N-[(3R)-5-[[4-(5-cyclopropyl-1,2,4-oxadiazol-3-yl)phenyl]methyl]-4-oxo-7-[5-(1,2,2,2-tetrafluoroethyl)-1,3,4-oxadiazol-2-yl]-2,3-dihydro-1,5-benzothiazepin-3-yl]carbamate (22.4 mg, 33.9 mol) in analogy to general procedure 10 and was obtained as colorless solid (13 mg, 53%) as colorless solid. MS (ESI): 691.3 [M−H]⁻

### Step 6: (3R)-3-amino-5-[[4-(5-cyclopropyl-1,2,4-oxadiazol-3-yl)phenyl]methyl]-1,1-dioxo-7-[5-(1,2,2,2-tetrafluoroethyl)-1,3,4-oxadiazol-2-yl]-2,3-dihydro-1λ⁶,5-benzothiazepin-4-one

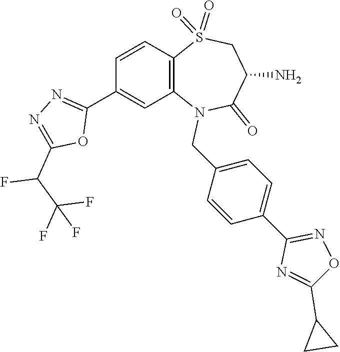 [SMILES: N[C@H]1CS(=O)(=O)c2ccc(-c3nnc(C(F)C(F)(F)F)o3)cc2N(Cc2ccc(-c3noc(C4CC4)n3)cc2)C1=O]

The title compound was prepared from tert-butyl N-[(3R)-5-[[4-(5-cyclopropyl-1,2,4-oxadiazol-3-yl)phenyl]methyl]-1,1,4-trioxo-7-[5-(1,2,2,2-tetrafluoroethyl)-1,3,4-oxadiazol-2-yl]-2,3-dihydro-1λ⁶,5-benzothiazepin-3-yl]carbamate (13 mg, 18.77 mol) in analogy to general procedure 11a and was obtained as white solid (12 mg, 98%), as hydrochloride salt. MS (ESI): 593.2 [M+H]⁺

The examples of the following table were prepared in analogy to Example 168, using the appropriate carboxylic acid building block.

| | | | | MS, |
|---|---|---|---|---|
| | | | | ESI: |
| Ex. | Structure | Systematic Name | Building Block | m/z |
| 169 | 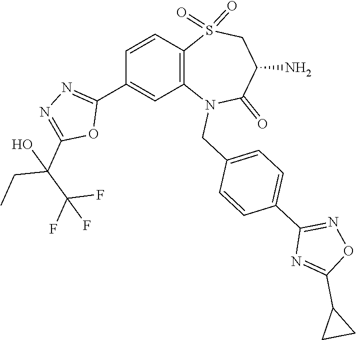 [SMILES: CCC(O)(c1nnc(-c2ccc3c(c2)N(Cc2ccc(-c4noc(C5CC5)n4)cc2)C(=O)[C@@H](N)CS3(=O)=O)o1)C(F)(F)F] | (3R)-3-amino-5-[4-(5- cyclopropyl-1,2,4- oxadiazol-3- yl)phenyl]methyl]-7-[5- [1-hydroxy-1- (trifluoromethyl)propyl]- 1,3,4-oxadiazol-2-yl]- 1,1-dioxo-2,3-dihydro- 1λ⁶,5-benzothiazepin-4- one (*) | 2-hydroxy-2- trifluoromethylbutyric acid (CAS 72114-82-0) | 619.3 [M + H]⁺ |
| 170 | 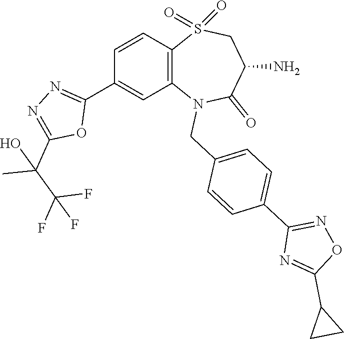 [SMILES: CC(O)(c1nnc(-c2ccc3c(c2)N(Cc2ccc(-c4noc(C5CC5)n4)cc2)C(=O)[C@@H](N)CS3(=O)=O)o1)C(F)(F)F] | (3R)-3-amino-5-[4-(5- cyclopropyl-1,2,4- oxadiazol-3- yl)phenyl]methyl]-1,1- dioxo-7-[5-(2,2,2- trifluoro-1-hydroxy-1- methyl-ethyl)-1,3,4- oxadiazol-2-yl]-2,3- dihydro-1λ⁶,5- benzothiazepin-4-one (*) | 3,3,3-trifluoro-2-hydroxy- 2-methylpropionic acid (CAS 374-35-6) | 605.3 [M + H]⁺ |
| 171 | 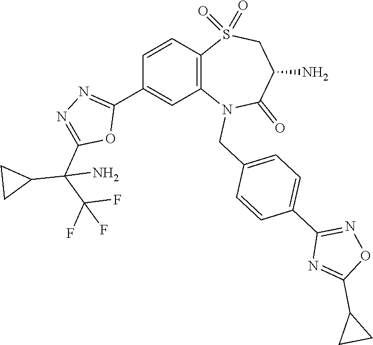 [SMILES: N[C@H]1CS(=O)(=O)c2ccc(-c3nnc(C(N)(C4CC4)C(F)(F)F)o3)cc2N(Cc2ccc(-c3noc(C4CC4)n3)cc2)C1=O] | (3R)-3-amino-7-[5-(1- amino-1-cyclopropyl- 2,2,2-trifluoro-ethyl)- 1,3,4-oxadiazol-2-yl]-5- [[4-(5-cyclopropyl- 1,2,4-oxadiazol-3- yl)phenyl]methyl]-1,1- dioxo-2,3-dihydro-1λ⁶,5- benzothiazepin-4-one (*) | 2-{[(tert- butoxy)carbonlyl]amino}- 2-cyclopropyl-3,3,3- trifluoropropanoic acid (CAS 2248296-37-7) | 630.3 [M + H]⁺ |

(*) as hydrochloride salt

### Example 172

### 1-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]cyclobutanecarbonitrile

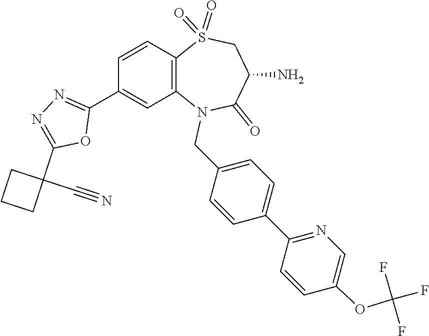 [SMILES: N#CC1(c2nnc(-c3ccc4c(c3)N(Cc3ccc(-c5ccc(OC(F)(F)F)cn5)cc3)C(=O)[C@@H](N)CS4(=O)=O)o2)CCC1]

### Step a) tert-butyl N-[(3R)-7-[[(]-cyanocyclobutanecarbonyl)amino]carbamoyl]-4-oxo-3,5-dihydro-2H-1,5-benzothiazepin-3-yl]carbamate

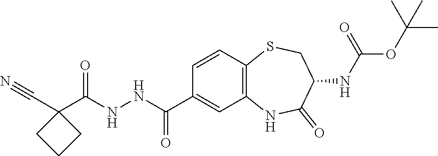 [SMILES: CC(C)(C)OC(=O)N[C@H]1CSc2ccc(C(=O)NNC(=O)C3(C#N)CCC3)cc2NC1=O]

The title compound was prepared from tert-butyl N-[(3R)-7-(hydrazinecarbonyl)-4-oxo-3,5-dihydro-2H-1,5-benzothiazepin-3-yl]carbamate (200 mg, 567.52 μmol, 1.0 eq, Example 19, step b) (70 mg, 198.6 μmol, 1.0 equiv) and 1-cyanocyclobutanecarboxylic acid (29.8 mg, 238.4 μmol, 1.2 eq) in analogy to general procedure 4a and was obtained as white solid (48 mg, 52%). MS (ESI): 404.2 [M+H-isobutene]⁺

### Step b) tert-butyl N-[(3R)-7-[5-(1-cyanocyclobutyl)-1,3,4-oxadiazol-2-yl]-4-oxo-3,5-dihydro-2H-1,5-benzothiazepin-3-yl]carbamate

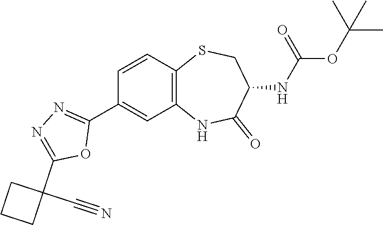 [SMILES: CC(C)(C)OC(=O)N[C@H]1CSc2ccc(-c3nnc(C4(C#N)CCC4)o3)cc2NC1=O]

The title compound was prepared from tert-butyl N-[(3R)-7-[[(1-cyanocyclobutanecarbonyl)amino]carbamoyl]-4-oxo-3,5-dihydro-2H-1,5-benzothiazepin-3-yl]carbamate (48 mg, 104.5 mol) in analogy to general procedure 5b and was obtained as white solid (25.7 mg, 54%). MS (ESI): 386.2 [M+H-isobutene]⁺

### Step c) tert-butyl N-[(3R)-7-[5-(1-cyanocyclobutyl)-1,3,4-oxadiazol-2-yl]-1,1,4-trioxo-3,5-dihydro-2H-1λ⁶,5-benzothiazepin-3-yl]carbamate

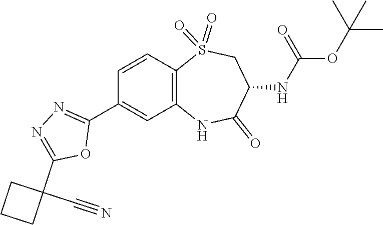 [SMILES: CC(C)(C)OC(=O)N[C@H]1CS(=O)(=O)c2ccc(-c3nnc(C4(C#N)CCC4)o3)cc2NC1=O]

The title compound was prepared from tert-butyl N-[(3R)-7-[5-(1-cyanocyclobutyl)-1,3,4-oxadiazol-2-yl]-4-oxo-3,5-dihydro-2H-1,5-benzothiazepin-3-yl]carbamate (25.7 mg, 58.2 mol) in analogy to general procedure 10 and was obtained as white solid (20.7 mg, 74%). MS (ESI): 418.1 [M+H-isobutene]⁺

### Step d) tert-butyl N-[(3R)-7-[5-(1-cyanocyclobutyl)-1,3,4-oxadiazol-2-yl]-1,1,4-trioxo-5-[[4-[5-(trifluoromethoxy)-2-pyridyl]phenyl]methyl-2,3-dihydro-1λ⁶, 5-benzothiazepin-3-yl]carbamate

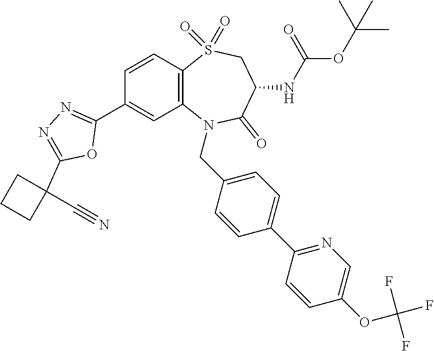 [SMILES: CC(C)(C)OC(=O)N[C@H]1CS(=O)(=O)c2ccc(-c3nnc(C4(C#N)CCC4)o3)cc2N(Cc2ccc(-c3ccc(OC(F)(F)F)cn3)cc2)C1=O]

The title compound was prepared from tert-butyl N-[(3R)-7-[5-(1-cyanocyclobutyl)-1,3,4-oxadiazol-2-yl]-1,1,4-trioxo-3,5-dihydro-2H-1λ⁶,5-benzothiazepin-3-yl]carbamate (20.7 mg, 43.7 μmol, 1.0 eq) and Intermediate 38 (19.6 mg, 59.0 μmol, 1.35 eq) in analogy to general procedure 1a and was obtained as white solid (29.3 mg, 88%). MS (ESI): 725.3 [M+H]⁺

### Step e) 1-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]cyclobutanecarbonitrile

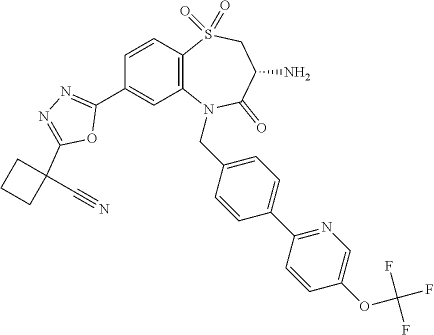 [SMILES: N#CC1(c2nnc(-c3ccc4c(c3)N(Cc3ccc(-c5ccc(OC(F)(F)F)cn5)cc3)C(=O)[C@@H](N)CS4(=O)=O)o2)CCC1]

The title compound was prepared from tert-butyl N-[(3R)-7-[5-(1-cyanocyclobutyl)-1,3,4-oxadiazol-2-yl]-1,1,4-trioxo-5-[[4-[5-(trifluoromethoxy)-2-pyridyl]phenyl]methyl]-2,3-dihydro-1λ⁶,5-benzothiazepin-3-yl]carbamate (29 mg, 40 mol) in analogy to general procedure 11a and was obtained as white solid (12.6 mg, 45%), as hydrochloride salt. MS (ESI): 625.3 [M+H]⁺

The examples of the following table were prepared in analogy to Example 172, using the appropriate carboxylic acid building block.

| | | | | MS, |
|---|---|---|---|---|
| | | | | ESI: |
| Ex. | Structure | Systematic Name | Building Block | m/z |
| 173 | 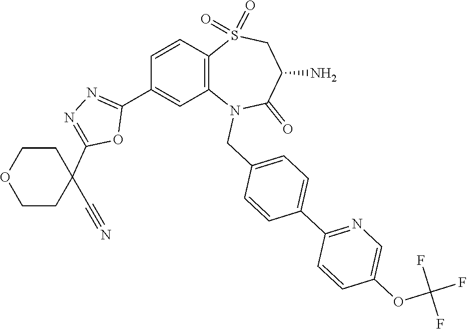 [SMILES: N#CC1(c2nnc(-c3ccc4c(c3)N(Cc3ccc(-c5ccc(OC(F)(F)F)cn5)cc3)C(=O)[C@@H](N)CS4(=O)=O)o2)CCOCC1] | 4-[5-[(3R)-3-amino- 1,1,4-trioxo-5-[[4- [5- (trifluoromethoxy)- 2- pyridyl]phenyl] methyl]-2,3-dihydro- 1λ⁶,5- benzothiazepin-7- yl]-1,3,4-oxadiazol- 2- yl]tetrahydropyran- 4-carbonitrile (*) | 4-cyanotetrahdro- 2H-pyran-4- carboxylic acid (CAS 848821-06- 7) | 655.1 [M + H]⁺ |
| 174 | 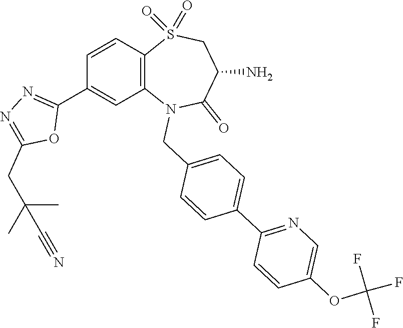 [SMILES: CC(C)(C#N)Cc1nnc(-c2ccc3c(c2)N(Cc2ccc(-c4ccc(OC(F)(F)F)cn4)cc2)C(=O)[C@@H](N)CS3(=O)=O)o1] | 3-[5-[(3R)-3-amino- 1, 1,4-trioxo-5-[[4- [5- (trifluoromethoxy)- 2- pyridyl]phenyl] methyl]-2,3-dihydro- 1λ⁶,5- benzothiazepin-7- yl]-1,3,4-oxadiazol- 2-yl]-2,2-dimethyl- propanenitrile (*) | 3-cyano-3- methylbutanoic acid (CAS 99839- 17-5) | 627.1 [M + H]⁺ |
| 175 | 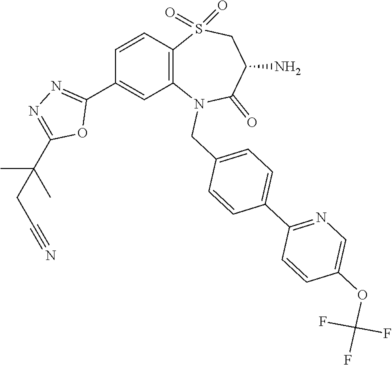 [SMILES: CC(C)(CC#N)c1nnc(-c2ccc3c(c2)N(Cc2ccc(-c4ccc(OC(F)(F)F)cn4)cc2)C(=O)[C@@H](N)CS3(=O)=O)o1] | 3-[5-[(3R)-3-amino- 1, 1,4-trioxo-5-[[4- [5- (trifluoromethoxy)- 2- pyridyl]phenyl] methyl]-2,3-dihydro- 1λ⁶,5- benzothiazepin-7- yl]-1,3,4-oxadiazol- 2-yl]-3-methyl- butanenitrile (*) | 3-cyano-2,2,- diemthylpropanoic acid (CAS 192327- 00-7) | 627.3 [M + H]⁺ |
| 176 | 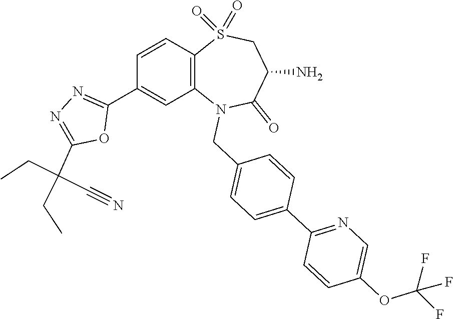 [SMILES: CCC(C#N)(CC)c1nnc(-c2ccc3c(c2)N(Cc2ccc(-c4ccc(OC(F)(F)F)cn4)cc2)C(=O)[C@@H](N)CS3(=O)=O)o1] | 2-[5-[(3R)-3-amino- 1,1,4-trioxo-5-[[4- [5- (trifluoromethoxy)- 2- pyridyl]phenyl] methyl]-2,3-dihydro- 1λ⁶,5- benzothiazepin-7- yl]-1,3,4-oxadiazol- 2-yl]-2-ethyl- butanenitrile (*) | 2-cyano-2- ethylbutanoic acid (CAS 4386-07-6) | 641.4 [M + H]⁺ |
| 177 | 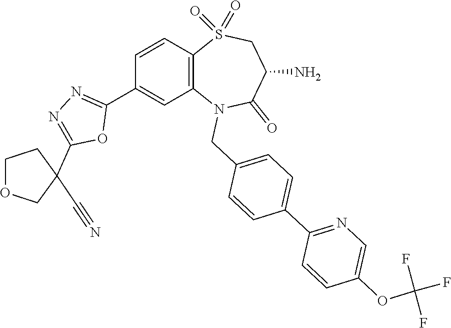 [SMILES: N#CC1(c2nnc(-c3ccc4c(c3)N(Cc3ccc(-c5ccc(OC(F)(F)F)cn5)cc3)C(=O)[C@@H](N)CS4(=O)=O)o2)CCOC1] | 3-[5-[(3R)-3-amino- 1,1,4-trioxo-5-[[4- [5- (trifluoromethoxy)- 2- pyridyl]phenyl] methyl]-2,3-dihydro- 1λ⁶,5- benzothiazepin-7- yl]-1,3,4-oxadiazol- 2- yl]tetrahydrofuran- 3-carbonitrile (*) | 3-cyanotetrahydro- 3-furancarboxylic acid (CAS 1500784-28-0) | 641.1 [M + H]⁺ |
| 178 | 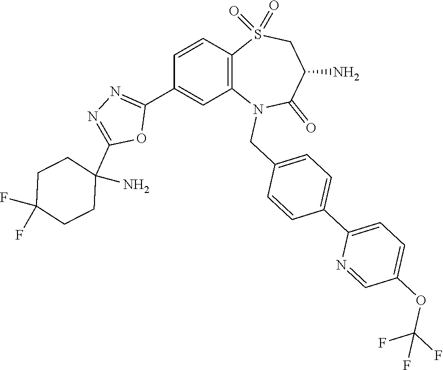 [SMILES: N[C@H]1CS(=O)(=O)c2ccc(-c3nnc(C4(N)CCC(F)(F)CC4)o3)cc2N(Cc2ccc(-c3ccc(OC(F)(F)F)cn3)cc2)C1=O] | (3R)-3-amino-7-[5- (1-amino-4,4- difluoro- cyclohexyl)-1,3,4- oxadiazol-2-yl]-1,1- dioxo-5-[[4-[5- (trifluoromethoxy)- 2- pyridyl]phenyl] methyl]-2,3-dihydro- 1λ⁶,5- benzothiazepin-4- one (*) | 1-[[(1,1- dimethylethoxy) carbonyl]amino]-3,3- difluorocyclohexane- carboxylic acid (CAS 1196151-58- 2) | 679.2 [M + H]⁺ |
| 179 | 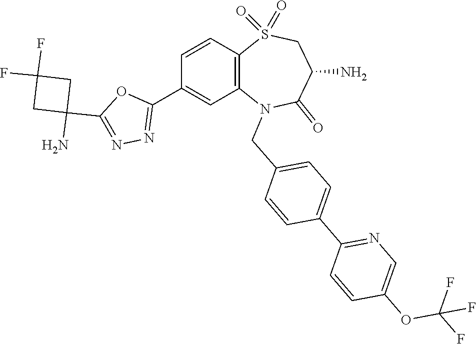 [SMILES: N[C@H]1CS(=O)(=O)c2ccc(-c3nnc(C4(N)CC(F)(F)C4)o3)cc2N(Cc2ccc(-c3ccc(OC(F)(F)F)cn3)cc2)C1=O] | (3R)-3-amino-7-[5- (1-amino-3,3- difluoro- cyclobutyl)-1,3,4- oxadiazol-2-yl]-1,1- dioxo-5-[[4-[5- (trifluoromethoxy)- 2- pyridyl]phenyl] methyl]-2,3-dihydro- 1λ⁶,5- benzothiazepin-4- one (*) | 1-[[(1,1- dimethylethoxy) carbonyl]amino]-3,3- difluorocyclobutane- carboxylic acid (CAS 1363380-83- 9) | 651.2 [M + H]⁺ |

(*) as hydrochloride salt

### Example 180

### 4-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-1-methyl-piperidine-4-carbonitrile

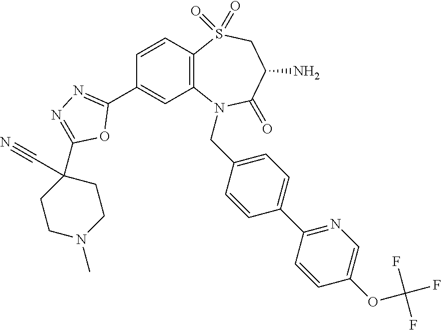 [SMILES: CN1CCC(C#N)(c2nnc(-c3ccc4c(c3)N(Cc3ccc(-c5ccc(OC(F)(F)F)cn5)cc3)C(=O)[C@@H](N)CS4(=O)=O)o2)CC1]

### Step a) benzyl 4-[[[(3R)-3-(tert-butoxycarbonylamino)-4-oxo-3,5-dihydro-2H-1,5-benzothiazepine-7-carbonyl]amino]carbamoyl]-4-cyano-piperidine-1-carboxylate

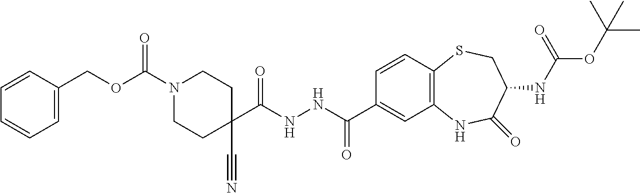 [SMILES: CC(C)(C)OC(=O)N[C@H]1CSc2ccc(C(=O)NNC(=O)C3(C#N)CCN(C(=O)OCc4ccccc4)CC3)cc2NC1=O]

The title product was prepared from tert-butyl N-[(3R)-7-(hydrazinecarbonyl)-4-oxo-3,5-dihydro-2H-1,5-benzothiazepin-3-yl]carbamate (74.4 mg, 211.2 μmol, Example 19, step b) in analogy to general procedure 4a and was obtained as off-white solid (58.9 mg, 47%). MS (ESI): 523.2 [M+H-Boc]⁺

### Step b) benzyl 4-[5-[(3R)-3-(tert-butoxycarbonylamino)-4-oxo-3,5-dihydro-2H-1,5-benzothiazepin-7-yl]-1,3,4-oxadiazol-2-yl]-4-cyano-piperidine-1-carboxylate

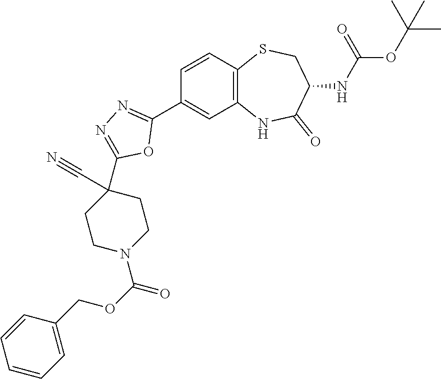 [SMILES: CC(C)(C)OC(=O)N[C@H]1CSc2ccc(-c3nnc(C4(C#N)CCN(C(=O)OCc5ccccc5)CC4)o3)cc2NC1=O]

The title compound was prepared from benzyl 4-[[[(3R)-3-(tert-butoxycarbonylamino)-4-oxo-3,5-dihydro-2H-1,5-benzothiazepine-7-carbonyl]amino]carbamoyl]-4-cyano-piperidine-1-carboxylate (58.9 mg, 94.6 mol) in analogy to general procedure 5b and was obtained as white solid (42.6 mg, 74%). MS (ESI): 549.2 [M+H-isobutene]⁺

### Step c) benzyl 4-[5-[(3R)-3-(tert-butoxycarbonylamino)-1,1,4-trioxo-3,5-dihydro-2H-16,5-benzothiazepin-7-yl]-1,3,4-oxadiazol-2-yl]-4-cyano-piperidine-1-carboxylate

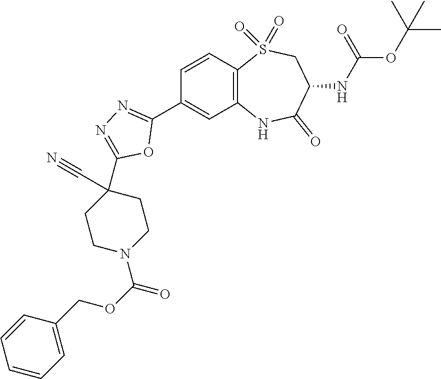 [SMILES: CC(C)(C)OC(=O)N[C@H]1CS(=O)(=O)c2ccc(-c3nnc(C4(C#N)CCN(C(=O)OCc5ccccc5)CC4)o3)cc2NC1=O]

The title compound was prepared from benzyl 4-[5-[(3R)-3-(tert-butoxycarbonylamino)-4-oxo-3,5-dihydro-2H-1,5-benzothiazepin-7-yl]-1,3,4-oxadiazol-2-yl]-4-cyano-piperidine-1-carboxylate (43.6 mg, 72 mol) in analogy to general procedure 10 and was obtained as white solid (32.6 mg, 70%). MS (ESI): 635.2 [M−H]⁻

### Step d) benzyl 4-[5-[(3R)-3-(tert-butoxycarbonylamino)-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-4-cyano-piperidine-]-carboxylate

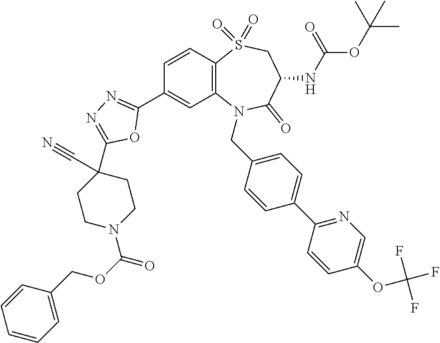 [SMILES: CC(C)(C)OC(=O)N[C@H]1CS(=O)(=O)c2ccc(-c3nnc(C4(C#N)CCN(C(=O)OCc5ccccc5)CC4)o3)cc2N(Cc2ccc(-c3ccc(OC(F)(F)F)cn3)cc2)C1=O]

The title compound was prepared from benzyl 4-[5-[(3R)-3-(tert-butoxycarbonylamino)-1,1,4-trioxo-3,5-dihydro-2H-1λ⁶,5-benzothiazepin-7-yl]-1,3,4-oxadiazol-2-yl]-4-cyano-piperidine-1-carboxylate (32.6 mg, 51.2 μmol, 1.0 eq) and Intermediate 38 (20.4 mg, 61.4 μmol, 1.2 eq) in analogy to general procedure 1a and was obtained as light yellow solid (40 mg, 87%). MS (ESI): 888.5 [M+H]⁺

### Step e) tert-butyl N-[(3R)-7-[5-(4-cyano-4-piperidyl)-1,3,4-oxadiazol-2-yl]-1,1,4-trioxo-5-[[4-[5-(trifluoromethoxy)-2-pyridyl]phenyl]methyl]-2,3-dihydro-1λ⁶, 5-benzothiazepin-3-yl]carbamate

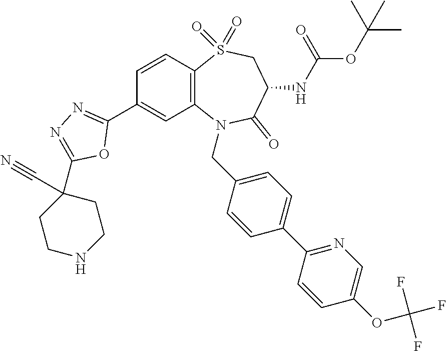 [SMILES: CC(C)(C)OC(=O)N[C@H]1CS(=O)(=O)c2ccc(-c3nnc(C4(C#N)CCNCC4)o3)cc2N(Cc2ccc(-c3ccc(OC(F)(F)F)cn3)cc2)C1=O]

Benzyl 4-[5-[(3R)-3-(tert-butoxycarbonylamino)-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-4-cyano-piperidine-1-carboxylate (40 mg, 45.1 μmol, 1.0 eq) was dissolved in MeOH (5 mL) under argon atmosphere. Pd/C (4.81 mg, 4.5 μmol, 0.1 eq) was added in one portion. The atmosphere was changed to hydrogen by the use of a hydrogen balloon (1 atm) and stirred for 16 h. The mixture was filtered directly over celite and the filter cake washed with MeOH. The filtrate was concentrated under reduced pressure to afford the title compound as white solid (27 mg, 71%). MS (ESI): 754.5 [M+H]⁺

### Step f) tert-butyl N-[(3R)-7-[5-(4-cyano-1-methyl-4-piperidyl)-], 3,4-oxadiazol-2-yl]-1,1,4-trioxo-5-[[[4-[5-(trifluoromethoxy)-2-pyridyl]phenyl]methyl]-2,3-dihydro-1λ⁶, 5-benzothiazepin-3-yl]carbamate

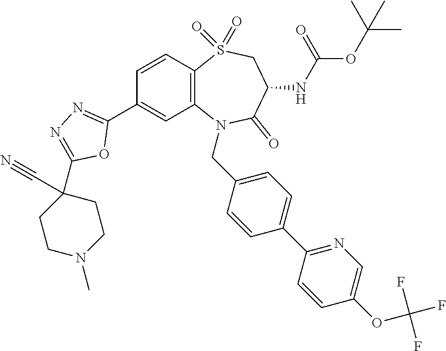 [SMILES: CN1CCC(C#N)(c2nnc(-c3ccc4c(c3)N(Cc3ccc(-c5ccc(OC(F)(F)F)cn5)cc3)C(=O)[C@@H](NC(=O)OC(C)(C)C)CS4(=O)=O)o2)CC1]

To a solution of tert-butyl N-[(3R)-7-[5-(4-cyano-4-piperidyl)-1,3,4-oxadiazol-2-yl]-1,1,4-trioxo-5-[[4-[5-(trifluoromethoxy)-2-pyridyl]phenyl]methyl]-2,3-dihydro-1λ⁶,5-benzothiazepin-3-yl]carbamate (27 mg, 35.8 μmol, 1.0 eq) in MeOH (0.44 ml) was added formaldehyde (37% in H₂O) (29.07 mg, 26.67 uL, 358.2 μmol, 10.0 eq) and sodium triacetoxyborohydride (75.9 mg, 358.2 μmol, 10.0 eq). After stirring for 1 h at room temperature, the reaction mixture was concentrated under reduced pressure and directly purified via prep. HPLC to afford the title compound (10.1 mg, 36%) as white solid. MS (ESI): 766.2 [M−H]⁻

### Step g) 4-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-1-methyl-piperidine-4-carbonitrile

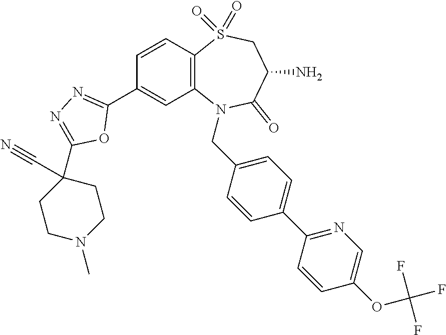 [SMILES: CN1CCC(C#N)(c2nnc(-c3ccc4c(c3)N(Cc3ccc(-c5ccc(OC(F)(F)F)cn5)cc3)C(=O)[C@@H](N)CS4(=O)=O)o2)CC1]

The title compound was prepared from tert-butyl N-[(3R)-7-[5-(4-cyano-1-methyl-4-piperidyl)-1,3,4-oxadiazol-2-yl]-1,1,4-trioxo-5-[[4-[5-(trifluoromethoxy)-2-pyridyl]phenyl]methyl]-2,3-dihydro-1λ⁶,5-benzothiazepin-3-yl]carbamate (10.1 mg, 13.2 mol) in analogy to general procedure 11a and was obtained as off-white solid (8.9 mg, 94%), as hydrochloride salt. MS (ESI): 668.2 [M+H]⁺

### Example 181

### (3R)-3-amino-5-[[4-(5-tert-butyl-1,2,4-oxadiazol-3-yl)phenyl]methyl]-7-[5-(1-ethyl-5,5-difluoro-3-piperidyl)-1,3,4-oxadiazol-2-yl]-1,1-dioxo-2,3-dihydro-1⁶,5-benzothiazepin-4-one

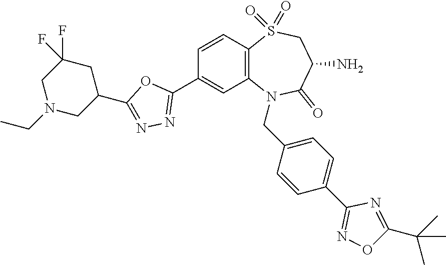 [SMILES: CCN1CC(c2nnc(-c3ccc4c(c3)N(Cc3ccc(-c5noc(C(C)(C)C)n5)cc3)C(=O)[C@@H](N)CS4(=O)=O)o2)CC(F)(F)C1]

### Step a) Methyl (3R)-3-(tert-butoxycarbonylamino)-5-[(4-cyanophenyl)methyl]-4-oxo-2,3-dihydro-1,5-benzothiazepine-7-carboxylate

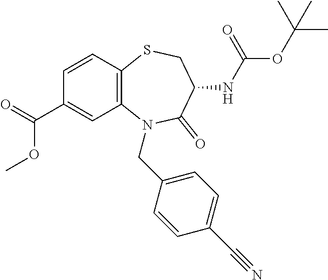 [SMILES: COC(=O)c1ccc2c(c1)N(Cc1ccc(C#N)cc1)C(=O)[C@@H](NC(=O)OC(C)(C)C)CS2]

The title compound was prepared from methyl (3R)-3-(tert-butoxycarbonylamino)-4-oxo-3,5-dihydro-2H-1,5-benzothiazepine-7-carboxylate (1000 mg, 2.84 mmol, 1.0 eq, CAS 2089150-67-7) and 4-(bromomethyl)benzonitrile (556.28 mg, 2.84 mmol, 1.0 eq, CAS 17201-43-3) in analogy to general procedure 1a and was obtained as yellow solid (1300 mg, 2.78 mmol, 98% yield). MS (ESI): 412.1 [M+H-isobutene]⁺

### Step b) (3R)-3-(tert-butoxycarbonylamino)-5-[(4-cyanophenyl)methyl]-4-oxo-2,3-dihydro-1,5-benzothiazepine-7-carboxylic acid

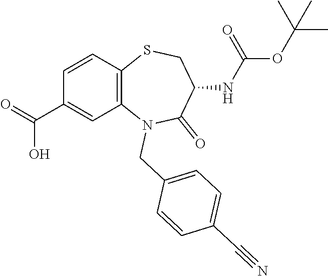 [SMILES: CC(C)(C)OC(=O)N[C@H]1CSc2ccc(C(=O)O)cc2N(Cc2ccc(C#N)cc2)C1=O]

To a solution of methyl (3R)-3-(tert-butoxycarbonylamino)-5-[(4-cyanophenyl)methyl]-1,1,4-trioxo-2,3-dihydro-1λ⁶,5-benzothiazepine-7-carboxylate (250 mg, 0.5 mmol, 1.0 eq) in THF (4 mL) and water (2 mL) was added LiBr (217 mg, 2.5 mmol, 5.0 eq) and triethylamine (253 mg, 2.5 mmol, 5.0 eq) at room temperature and the mixture was stirred at 50° C. for 48 h. The reaction mixture was cooled to room temperature and carefully acidified with 0.5 N HCl to pH=4-5 and extracted with EtOAc (50 mL×3). The combined extracts were washed with brine (30 mL), dried over sodium sulfate, filtered, and concentrated under reduced pressure to give (3R)-3-(tert-butoxycarbonylamino)-5-[(4-cyanophenyl)methyl]-1,1,4-trioxo-2,3-dihydro-1λ⁶,5-benzothiazepine-7-carboxylic acid (275 mg, 0.389 mmol, 78% yield) as light yellow solid. MS (ESI): 484.0 [M−H]⁻

### Step c) (3R)-3-(tert-butoxycarbonylamino)-5-[(4-cyanophenyl)methyl]-1,1,4-trioxo-2,3-dihydro-1λ⁶, 5-benzothiazepine-7-carboxylic acid

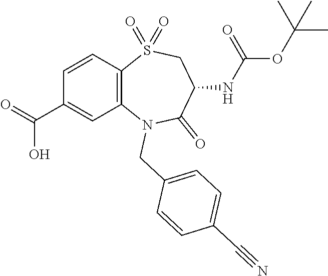 [SMILES: CC(C)(C)OC(=O)N[C@H]1CS(=O)(=O)c2ccc(C(=O)O)cc2N(Cc2ccc(C#N)cc2)C1=O]

To a solution of NaIO₄ (1084 mg, 5.07 mmol, 2.0 eq) in water (6 mL) was added RuCl₃ (52.6 mg, 0.25 mmol, 0.1 eq) in small portions at 0° C. and the mixture was stirred for 10 min. Then, a solution of 3R)-3-(tert-butoxycarbonylamino)-5-[(4-cyanophenyl)methyl]-4-oxo-2,3-dihydro-1,5-benzothiazepine-7-carboxylic acid (1150 mg, 2.54 mmol, 1.0 eq) in MeCN (10 mL) was added dropwise at 0° C. After complete addition, the mixture was allowed to stir at room temperature for 2 h. The reaction was quenched by addition of i-PrOH (5 mL) and stirring for 30 min. The reaction was diluted with EtAOc (50 mL) and filtered through celite. The filtrate was washed with water (50 mL) and the phases were separated. The aqueous phase was extracted with EtOAc (50 mL×3). The combined organic extracts were washed with brine (40 mL), dried with anhydrous sodium sulfate, filtered and concentrated in vacuum to afford (3R)-3-(tert-butoxycarbonylamino)-5-[(4-cyanophenyl)methyl]-1,1,4-trioxo-2,3-dihydro-1λ⁶,5-benzothiazepine-7-carboxylic acid (1200 mg, 2.47 mmol, 97% yield) as light yellow solid, MS (ESI): 430.1 [M+H-isobutene]⁺

### Step d) tert-butyl N-[(3R)-5-[(4-cyanophenyl)methyl]-7-(hydrazinecarbonyl)-1,1,4-trioxo-2,3-dihydro-1λ⁶, 5-benzothiazepin-3-yl]carbamate

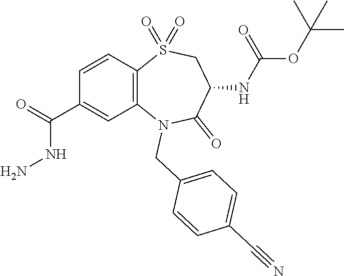 [SMILES: CC(C)(C)OC(=O)N[C@H]1CS(=O)(=O)c2ccc(C(=O)NN)cc2N(Cc2ccc(C#N)cc2)C1=O]

The title compound was prepared from (3R)-3-(tert-butoxycarbonylamino)-5-[(4-cyanophenyl)methyl]-1,1,4-trioxo-2,3-dihydro-1λ⁶,5-benzothiazepine-7-carboxylic acid (1200 mg, 2.4 mmol) in analogy to general procedure 3 and was obtained as light yellow oil (1100 mg, 2.2 mmol, 74% yield). MS (ESI): 444.1 [M+H-isobutene]⁺

### Step e) tert-butyl N-[(3R)-5-[(4-cyanophenyl)methyl]-7-[[(1-ethyl-5,5-difluoro-piperidine-3-carbonyl)amino]carbamoyl]-1,1,4-trioxo-2,3-dihydro-1λ⁶, 5-benzothiazepin-3-yl]carbamate

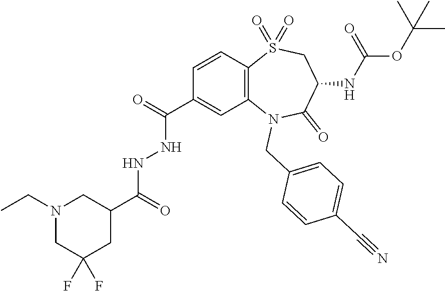 [SMILES: CCN1CC(C(=O)NNC(=O)c2ccc3c(c2)N(Cc2ccc(C#N)cc2)C(=O)[C@@H](NC(=O)OC(C)(C)C)CS3(=O)=O)CC(F)(F)C1]

The title compound was prepared from tert-butyl N-[(3R)-5-[(4-cyanophenyl)methyl]-7-(hydrazinecarbonyl)-1,1,4-trioxo-2,3-dihydro-1λ⁶,5-benzothiazepin-3-yl]carbamate (300 mg, 0.6 mmol, 1.0 eq) and 1-ethyl-5,5-difluoro-piperidine-3-carboxylic acid (232 mg, 1.2 mmol, 2.0 eq, CAS 2912473-22-2) in analogy to general procedure 4b and was obtained as yellow solid (360 mg, 0.53 mmol, 53% yield). MS (ESI): 675.4 [M+H]⁺

### Step f) tert-butyl N-[(3R)-5-[(4-cyanophenyl)methyl]-7-[5-(1-ethyl-5,5-difluoro-3-piperidyl)-1,3,4-oxadiazol-2-yl]-1,1,4-trioxo-2,3-dihydro-1λ⁶,5-benzothiazepin-3-yl]carbamate

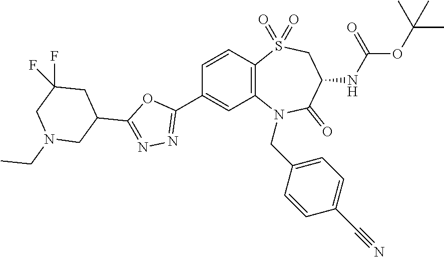 [SMILES: CCN1CC(c2nnc(-c3ccc4c(c3)N(Cc3ccc(C#N)cc3)C(=O)[C@@H](NC(=O)OC(C)(C)C)CS4(=O)=O)o2)CC(F)(F)C1]

The title compound was prepared from tert-butyl N-[(3R)-5-[(4-cyanophenyl)methyl]-7-[[(1-ethyl-5,5-difluoro-piperidine-3-carbonyl)amino]carbamoyl]-1,1,4-trioxo-2,3-dihydro-1λ⁶,5-benzothiazepin-3-yl]carbamate (360 mg, 0.53 mmol) in analogy to general procedure 5a and was obtained as yellow solid (270 mg, 0.41 mmol, 50% yield) as yellow solid. MS (ESI): 657.3 [M+H]⁺

### Step g) tert-butyl N-[(3R)-7-[5-(1-ethyl-5,5-difluoro-3-piperidyl)-1,3,4-oxadiazol-2-yl]-5-[[4-[(Z)-N′-hydroxycarbamimidoyl]phenyl]methyl]-1,1,4-trioxo-2,3-dihydro-1λ⁶,5-benzothiazepin-3-yl]carbamate

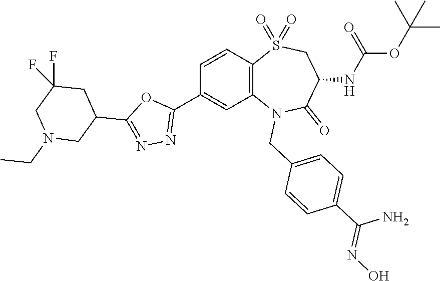 [SMILES: CCN1CC(c2nnc(-c3ccc4c(c3)N(Cc3ccc(/C(N)=N/O)cc3)C(=O)[C@@H](NC(=O)OC(C)(C)C)CS4(=O)=O)o2)CC(F)(F)C1]

To a suspension of tert-butyl N-[(3R)-5-[(4-cyanophenyl)methyl]-7-[5-(1-ethyl-5,5-difluoro-3-piperidyl)-1,3,4-oxadiazol-2-yl]-1,1,4-trioxo-2,3-dihydro-1λ⁶,5-benzothiazepin-3-yl]carbamate (270.0 mg, 0.41 mmol, 1.0 eq), K₂CO₃ (170 mg, 1.23 mmol, 3.0 eq) in EtOH (5 mL) was added hydroxylammonium chloride (42.86 mg, 0.62 mmol, 1.5 eq) and the mixture was stirred at 50° C. for 12 h. The mixture was allowed to cool to room temperature and then poured into water (50 mL). The mixture was extracted with EtOAc (40 mL×3). The combined organic layers were washed with with brine (100 mL), dried over sodium sulfate concentrated under reduced pressure to give the title compound (180 mg, 0.26 mmol, 43% yield) as yellow oil. MS (ESI): 690.2 [M+H]⁺

### Step h) [(Z)-[amino-[4-[[(3R)-3-(tert-butoxycarbonylamino)-7-[5-(1-ethyl-5,5-difluoro-3-piperidyl)-1,3,4-oxadiazol-2-yl]-1,1,4-trioxo-2,3-dihydro-1λ⁶,5-benzothiazepin-5-yl]methyl]phenyl]methylene]amino]2,2-dimethylpropanoate

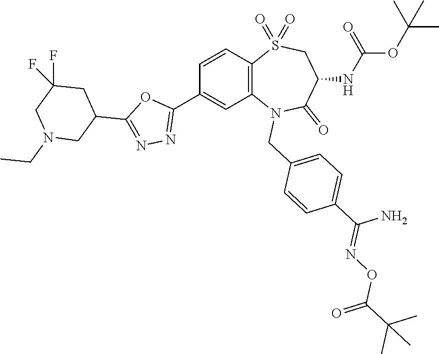 [SMILES: CCN1CC(c2nnc(-c3ccc4c(c3)N(Cc3ccc(/C(N)=N/OC(=O)C(C)(C)C)cc3)C(=O)[C@@H](NC(=O)OC(C)(C)C)CS4(=O)=O)o2)CC(F)(F)C1]

To a solution of tert-butyl N-[(3R)-7-[5-(1-ethyl-5,5-difluoro-3-piperidyl)-1,3,4-oxadiazol-2-yl]-1,1,4-trioxo-5-[[4-[(Z)-N′-hydroxycarbamimidoyl]phenyl]methyl]-2,3-dihydro-1λ⁶,5-benzothiazepin-3-yl]carbamate (170 mg, 0.25 mmol, 1.0 eq), triethylamine (0.1 mL, 0.74 mmol, 3.0 eq) in THF (5 mL) was added pivaloyl chloride (44.58 mg, 0.37 mmol, 1.5 eq) at room temperature and the mixture was stirred for 2 h. The solution was poured into water (20 mL) and the mixture extracted with EtOAc (15 mL×3). The combined organic phase was washed with brine (50 mL), dried over sodium sulfate, concentrated under reduced pressure to afford the title compound (190 mg, 0.25 mmol, 75% yield) as yellow oil. MS (ESI): 774.3 [M+H]⁺

### Step i) tert-butyl N-[(3R)-5-[[4-(5-tert-butyl-1,2,4-oxadiazol-3-yl)phenyl]methyl]-7-[5-(1-ethyl-5,5-difluoro-3-piperidyl)-1,3,4-oxadiazol-2-yl]-1,1,4-trioxo-2,3-dihydro-1λ⁶, 5-benzothiazepin-3-yl]carbamate

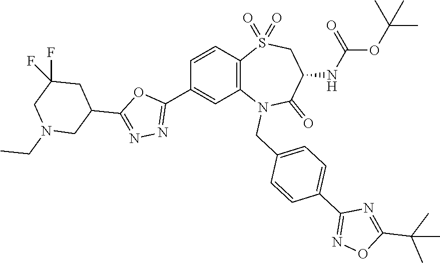 [SMILES: CCN1CC(c2nnc(-c3ccc4c(c3)N(Cc3ccc(-c5noc(C(C)(C)C)n5)cc3)C(=O)[C@@H](NC(=O)OC(C)(C)C)CS4(=O)=O)o2)CC(F)(F)C1]

A solution of [(Z)-[amino-[4-[[(3R)-3-(tert-butoxycarbonylamino)-7-[5-(1-ethyl-5,5-difluoro-3-piperidyl)-1,3,4-oxadiazol-2-yl]-1,1,4-trioxo-2,3-dihydro-1λ⁶,5-benzothiazepin-5-yl]methyl]phenyl]methylene]amino]2,2-dimethylpropanoate (190 mg, 0.25 mmol) in DMF (5 mL) was stirred at 120° C. for 12 h. The reaction mixture was directly purified by prep-HPLC to afford the title compound (70 mg, 0.09 mmol, 32% yield) as white solid. MS (ESI): 756.2 [M+H]⁺

### Step j) (3R)-3-amino-5-[[4-(5-tert-butyl-1,2,4-oxadiazol-3-yl)phenyl]methyl]-7-[5-(1-ethyl-5,5-difluoro-3-piperidyl)-1,3,4-oxadiazol-2-yl]-1,1-dioxo-2,3-dihydro-1λ⁶,5-benzothiazepin-4-one

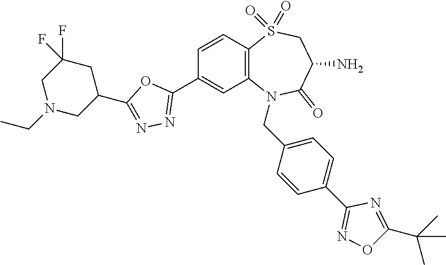 [SMILES: CCN1CC(c2nnc(-c3ccc4c(c3)N(Cc3ccc(-c5noc(C(C)(C)C)n5)cc3)C(=O)[C@@H](N)CS4(=O)=O)o2)CC(F)(F)C1]

The title compound was prepared from tert-butyl N-[(3R)-7-[5-(1-ethyl-5,5-difluoro-3-piperidyl)-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (50 mg, 0.07 mmol) in analogy to general procedure 11c and was obtained as yellow solid (15.2 mg, 0.02 mmol, 35% yield). MS (ESI): 656.2 [M+H]⁺

### Example 182

### (3R)-3-amino-7-[5-[5,5-difluoro-1-(2-methoxyethyl)-3-piperidyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

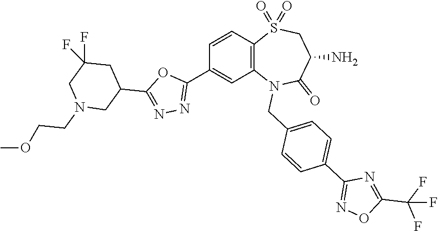 [SMILES: COCCN1CC(c2nnc(-c3ccc4c(c3)N(Cc3ccc(-c5noc(C(F)(F)F)n5)cc3)C(=O)[C@@H](N)CS4(=O)=O)o2)CC(F)(F)C1]

### Step a) tert-butyl N-[(3R)-5-[(4-cyanophenyl)methyl]-7-[[[5,5-difluoro-1-(2-methoxyethyl)piperidine-3-carbonyl]amino]carbamoyl]-1,1,4-trioxo-2,3-dihydro-1λ⁶,5-benzothiazepin-3-yl]carbamate

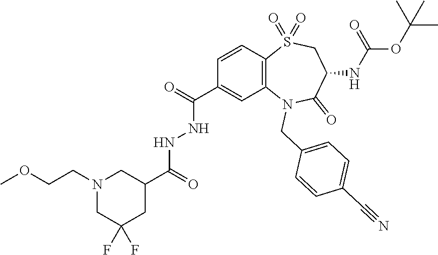 [SMILES: COCCN1CC(C(=O)NNC(=O)c2ccc3c(c2)N(Cc2ccc(C#N)cc2)C(=O)[C@@H](NC(=O)OC(C)(C)C)CS3(=O)=O)CC(F)(F)C1]

The title compound was prepared from tert-butyl N-[(3R)-5-[(4-cyanophenyl)methyl]-7-(hydrazinecarbonyl)-1,1,4-trioxo-2,3-dihydro-1λ⁶,5-benzothiazepin-3-yl]carbamate (140 mg, 0.28 mmol, 1.0 eq, Example 172, step d) and Intermediate 55 (125 mg, 0.56 mmol, 2.0 eq) in analogy to general procedure 4b and was obtained as yellow solid (160 mg, 0.23 mmol, 82% yield). MS (ESI): 705.3 [M+H]⁺

### Step b) tert-butyl N-[(3R)-5-[(4-cyanophenyl)methyl]-7-[5-[5,5-difluoro-1-(2-methoxyethyl)-3-piperidyl]-1,3,4-oxadiazol-2-yl]-1,1,4-trioxo-2,3-dihydro-1λ⁶,5-benzothiazepin-3-yl]carbamate

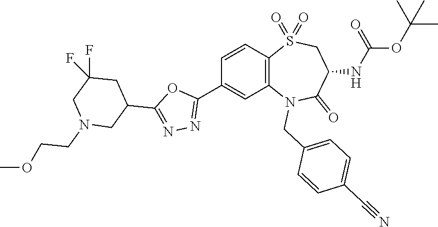 [SMILES: COCCN1CC(c2nnc(-c3ccc4c(c3)N(Cc3ccc(C#N)cc3)C(=O)[C@@H](NC(=O)OC(C)(C)C)CS4(=O)=O)o2)CC(F)(F)C1]

The title compound was prepared from tert-butyl N-[(3R)-5-[(4-cyanophenyl)methyl]-7-[[[5,5-difluoro-1-(2-methoxyethyl)piperidine-3-carbonyl]amino]carbamoyl]-1,1,4-trioxo-2,3-dihydro-1λ⁶,5-benzothiazepin-3-yl]carbamate (800 mg, 1.14 mmol, 1.0 eq) in analogy to general procedure 5a and was obtained as yellow solid (320 mg, 0.47 mmol, 41% yield). MS (ESI): 687.3 [M+H]⁺

### Step c) tert-butyl N-[(3R)-7-[5-[5,5-difluoro-1-(2-methoxyethyl)-3-piperidyl]-1,3,4-oxadiazol-2-yl]-5-[[4-[(Z)-N′-hydroxycarbamimidoyl]phenyl]methyl]-1,1,4-trioxo-2,3-dihydro-1λ⁶,5-benzothiazepin-3-yl]carbamate

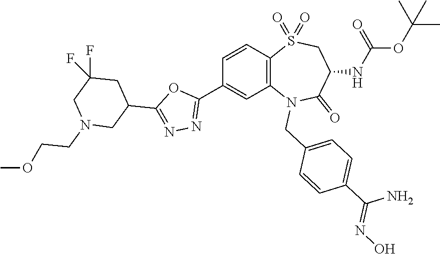 [SMILES: COCCN1CC(c2nnc(-c3ccc4c(c3)N(Cc3ccc(/C(N)=N/O)cc3)C(=O)[C@@H](NC(=O)OC(C)(C)C)CS4(=O)=O)o2)CC(F)(F)C1]

The title compound as prepared from tert-butyl N-[(3R)-5-[(4-cyanophenyl)methyl]-7-[5-[5,5-difluoro-1-(2-methoxyethyl)-3-piperidyl]-1,3,4-oxadiazol-2-yl]-1,1,4-trioxo-2,3-dihydro-1λ⁶,5-benzothiazepin-3-yl]carbamate (310 mg, 0.45 mmol, 1.0 eq) in analogy to example 172, step d and was obtained as yellow solid (260 mg, 0.36 mmol, 80% yield). MS (ESI): 720.3 [M+H]⁺

### Step d) tert-butyl N-[(3R)-7-[5-[5,5-difluoro-1-(2-methoxyethyl)-3-piperidyl]-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate

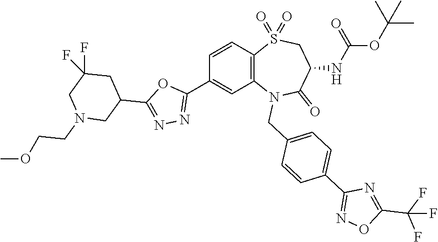 [SMILES: COCCN1CC(c2nnc(-c3ccc4c(c3)N(Cc3ccc(-c5noc(C(F)(F)F)n5)cc3)C(=O)[C@@H](NC(=O)OC(C)(C)C)CS4(=O)=O)o2)CC(F)(F)C1]

To a solution of tert-butyl N-[(3R)-7-[5-[5,5-difluoro-1-(2-methoxyethyl)-3-piperidyl]-1,3,4-oxadiazol-2-yl]-1,1,4-trioxo-5-[[4-[(Z)-N′-hydroxycarbamimidoyl]phenyl]methyl]-2,3-dihydro-1λ⁶,5-benzothiazepin-3-yl]carbamate (250 mg, 0.35 mmol, 1.0 eq) in THF (5 ml) at room temperature was added trifluoroacetic anhydride (0.07 mL, 0.52 mmol, 1.5 eq) and stirred for 3 h. The solution was poured into water (20 mL) and the pH was carefully adjusted to pH 7-8 by addition of solid NaHCO₃. The mixture was then extracted with EtOAc (15 mL×3). The combined organic phase was washed with brine (50 mL), dried over sodium sulfate and concentrated under reduce depressure. The remaining residue was purified by prep-HPLC to afford the title compound (60 mg, 0.08 mmol, 22% yield) as white solid. MS (ESI): 798.3 [M+H]⁺

### Step e) (3R)-3-amino-7-[5-[5,5-difluoro-1-(2-methoxyethyl)-3-piperidyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one

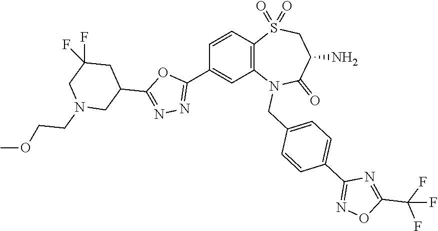 [SMILES: COCCN1CC(c2nnc(-c3ccc4c(c3)N(Cc3ccc(-c5noc(C(F)(F)F)n5)cc3)C(=O)[C@@H](N)CS4(=O)=O)o2)CC(F)(F)C1]

The title compound was prepared from tert-butyl N-[(3R)-7-[5-[5,5-difluoro-1-(2-methoxyethyl)-3-piperidyl]-1,3,4-oxadiazol-2-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (40 mg, 0.05 mmol) in analogy to general procedure 11c and was obtained as white solid (23.5 mg, 0.03 mmol, 63% yield), as hydrochloride salt. MS (ESI): 698.3 [M+H]⁺

### Example 183

### (3R)-3-amino-7-[5-[5,5-difluoro-1-(2-methoxyethyl)-3-piperidyl]-1,3,4-oxadiazol-2-yl]-5-[[4-(4-methoxyphenyl)phenyl]methyl]-1,1-dioxo-2,3-dihydro-1λ⁶, 5-benzothiazepin-4-one

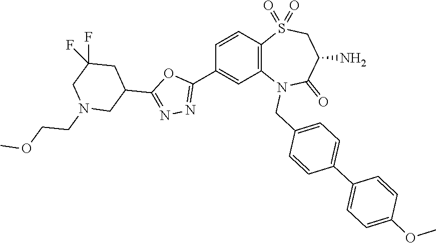

### Step a) methyl (3R)-3-(tert-butoxycarbonylamino)-5-[[4-(4-methoxyphenyl)phenyl]methyl]-4-oxo-2,3-dihydro-1,5-benzothiazepine-7-carboxylate

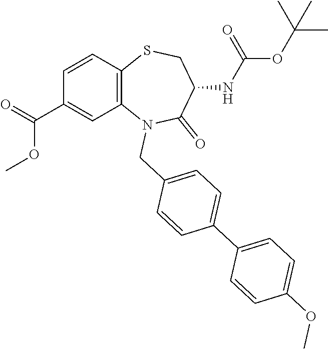 [SMILES: COC(=O)c1ccc2c(c1)N(Cc1ccc(-c3ccc(OC)cc3)cc1)C(=O)[C@@H](NC(=O)OC(C)(C)C)CS2]

The title compound was prepared from methyl (3R)-3-(tert-butoxycarbonylamino)-4-oxo-3,5-dihydro-2H-1,5-benzothiazepine-7-carboxylate (500 mg, 1.42 mmol, 1.0 eq, CAS 2089150-67-7) and 1-(chloromethyl)-4-(4-methoxyphenyl)benzene (396.2 mg, 1.7 mmol, 1.2 eq, CAS 93258-73-2) in analogy to general procedure 1a and was obtained as yellow oil (1600.0 mg, 2.92 mmol, 88% yield). MS (ESI). 493.1 [M+H-isobutene]³⁰

### Step b) (3R)-3-(tert-butoxycarbonylamino)-5-[[4-(4-methoxyphenyl)phenyl]methyl]-4-oxo-2,3-dihydro-1,5-benzothiazepine-7-carboxylic acid

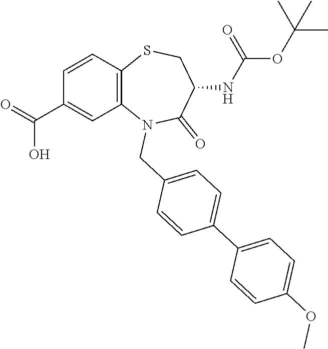 [SMILES: COc1ccc(-c2ccc(CN3C(=O)[C@@H](NC(=O)OC(C)(C)C)CSc4ccc(C(=O)O)cc43)cc2)cc1]

The title compound was prepared form methyl (3R)-3-(tert-butoxycarbonylamino)-5-[[4-(4-methoxyphenyl)phenyl]methyl]-4-oxo-2,3-dihydro-1,5-benzothiazepine-7-carboxylate (500 mg, 0.91 mmol) in analogy to general procedure 2 and was obtained as yellow oil (450 mg, 0.84 mmol, 92% yield). MS (ESI): 479.2 [M+H-isobutene]⁺

### Step c) (3R)-3-(tert-butoxycarbonylamino)-5-[[4-(4-methoxyphenyl)phenyl]methyl]-1,1,4-trioxo-2,3-dihydro-1λ⁶,5-benzothiazepine-7-carboxylic acid

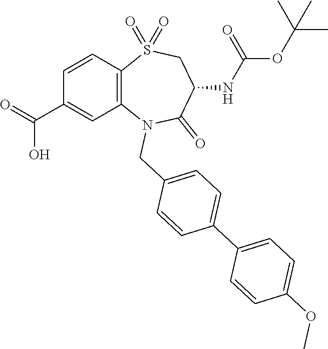 [SMILES: COc1ccc(-c2ccc(CN3C(=O)[C@@H](NC(=O)OC(C)(C)C)CS(=O)(=O)c4ccc(C(=O)O)cc43)cc2)cc1]

To a solution of NaIO₄ (360 mg, 1.68 mmol, 2.0 eq) in water (8 mL) was added RuCl₃ (17.5 mg, 0.08 mmol, 0.1 eq) in small portions at 0° C. and the mixture was stirred for 10 min. Then, a solution of (3R)-3-(tert-butoxycarbonylamino)-5-[[4-(4-methoxyphenyl)phenyl]methyl]-4-oxo-2,3-dihydro-1,5-benzothiazepine-7-carboxylic acid (450 mg, 0.84 mmol, 1.0 eq) in MeCN (8 mL) was added dropwise at 0° C. After complete addition, the mixture was allowed to stir at room temperature for 2 h. The reaction was quenched by addition of i-PrOH (5 mL) and stirring for 30 min. The reaction was diluted with EtAOc (50 mL) and filtered through celite. The filtrate was washed with water (50 mL) and the phases were separated. The aqueous phase was extracted with EtOAc (50 mL×3). The combined organic extracts were washed with brine (100 mL), dried with anhydrous sodium sulfate, filtered and concentrated in vacuum to afford (3R)-3-(tert-butoxycarbonylamino)-5-[[4-(4-methoxyphenyl)phenyl]methyl]-1,1,4-trioxo-2,3-dihydro-1λ⁶,5-benzothiazepine-7-carboxylic acid (370 mg, 0.65 mmol, 78% yield) as yellow solid. MS (ESI): 589.3.3 [M+Na]⁺

### Step d) tert-butyl N-[(3R)-7-(hydrazinecarbonyl)-5-[[4-(4-methoxyphenyl)phenyl]methyl]-1,1,4-trioxo-2,3-dihydro-1λ⁶,5-benzothiazepin-3-yl]carbamate

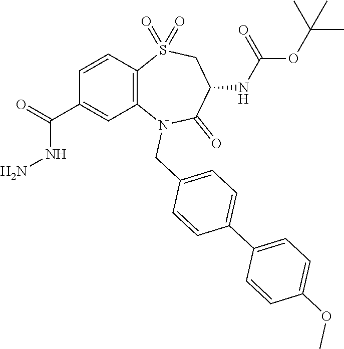 [SMILES: COc1ccc(-c2ccc(CN3C(=O)[C@@H](NC(=O)OC(C)(C)C)CS(=O)(=O)c4ccc(C(=O)NN)cc43)cc2)cc1]

The title compound was prepared from (3R)-3-(tert-butoxycarbonylamino)-5-[[4-(4-methoxyphenyl)phenyl]methyl]-1,1,4-trioxo-2,3-dihydro-1λ⁶,5-benzothiazepine-7-carboxylic acid (360 mg, 0.64 mmol) in analogy to general procedure 3 and was obtained as yellow solid (270 mg, 0.46 mmol, 73% yield). MS (ESI): 525.2 [M+H-isobutene]⁺

### Step e) tert-butyl N-[(3R)-7-[[[5,5-difluoro-1-(2-methoxyethyl)piperidine-3-carbonyl]amino]carbamoyl]-5-[[4-(4-methoxyphenyl)phenyl]methyl]-1,1,4-trioxo-2,3-dihydro-1λ⁶, 5-benzothiazepin-3-yl]carbamate

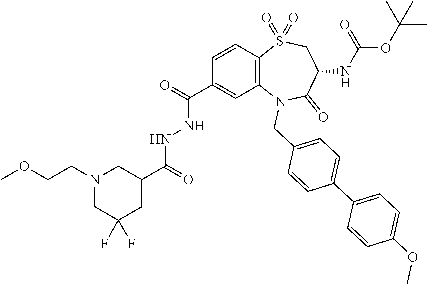 [SMILES: COCCN1CC(C(=O)NNC(=O)c2ccc3c(c2)N(Cc2ccc(-c4ccc(OC)cc4)cc2)C(=O)[C@@H](NC(=O)OC(C)(C)C)CS3(=O)=O)CC(F)(F)C1]

The title compound was prepared from tert-butyl N-[(3R)-7-(hydrazinecarbonyl)-5-[[4-(4-methoxyphenyl)phenyl]methyl]-1,1,4-trioxo-2,3-dihydro-1λ⁶,5-benzothiazepin-3-yl]carbamate (250 mg, 0.43 mmol, 1.0 eq) and Intermediate 55 (288 mg, 1.29 mmol, 3.0 eq) in analogy to general procedure 4b and was obtained as yellow solid (450 mg, 0.57 mmol, 57% yield) as yellow solid. MS (ESI): 786.4 [M+H]⁺

### Step f) tert-butyl N-[(3R)-7-[5-[5,5-difluoro-1-(2-methoxyethyl)-3-piperidyl]-1,3,4-oxadiazol-2-yl]-5-[[4-(4-methoxyphenyl)phenyl]methyl]-1,1,4-trioxo-2,3-dihydro-1λ⁶, 5-benzothiazepin-3-yl]carbamate

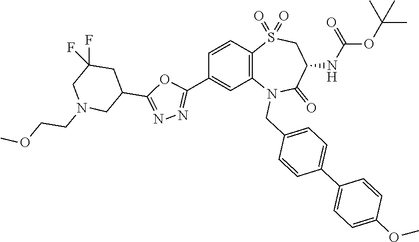

The title compound was prepared from tert-butyl N-[(3R)-7-[[[5,5-difluoro-1-(2-methoxyethyl)piperidine-3-carbonyl]amino]carbamoyl]-5-[[4-(4-methoxyphenyl)phenyl]methyl]-1,1,4-trioxo-2,3-dihydro-1λ⁶,5-benzothiazepin-3-yl]carbamate (200 mg, 0.25 mmol) in analogy to general procedure 5a and was obtained as white solid (55 mg, 0.07 mmol, 28% yield). MS (ESI): 768.4 [M+H]⁺

### Step g) (3R)-3-amino-7-[5-[5,5-difluoro-1-(2-methoxyethyl)-3-piperidyl]-1,3,4-oxadiazol-2-yl]-5-[[4-(4-methoxyphenyl)phenyl]methyl]-1,1-dioxo-2,3-dihydro-1λ⁶, 5-benzothiazepin-4-one

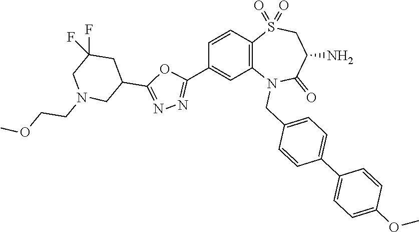

The title compound was prepared from tert-butyl N-[(3R)-7-[5-[5,5-difluoro-1-(2-methoxyethyl)-3-piperidyl]-1,3,4-oxadiazol-2-yl]-5-[[4-(4-methoxyphenyl)phenyl]methyl]-1,1,4-trioxo-2,3-dihydro-1λ⁶,5-benzothiazepin-3-yl]carbamate (50 mg, 0.07 mmol) in analogy to general procedure 11c and was obtained as yellow solid (28.5 mg, 0.04 mmol, 59% yield), as hydrochloride salt. MS (ESI): 668.2 [M+H]⁺

### Example 84

### (3R)-3-amino-7-[5-(3-aminooxetan-3-yl)-1,2,4-oxadiazol-3-yl]-1,1-dioxo-5-[[4-[5-(trifluoromethyl)-1,2,4-oxadiazol-3-yl]phenyl]methyl]-2,3-dihydro-1lambda6,5-benzothiazepin-4-one

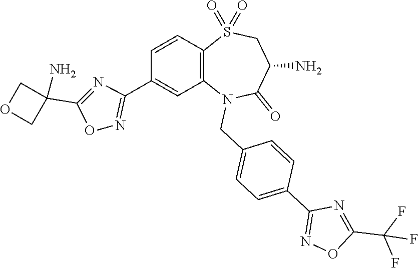 [SMILES: N[C@H]1CS(=O)(=O)c2ccc(-c3noc(C4(N)COC4)n3)cc2N(Cc2ccc(-c3noc(C(F)(F)F)n3)cc2)C1=O]

### Step a) tert-butyl N-[(3R)-7-[(Z)-N′-hydroxycarbamimidoyl]-4-oxo-3,5-dihydro-2H-1,5-benzothiazepin-3-yl]carbamate

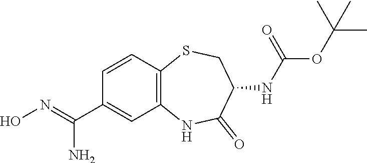 [SMILES: CC(C)(C)OC(=O)N[C@H]1CSc2ccc(/C(N)=N/O)cc2NC1=O]

The title compound was prepared from tert-butyl N-[(3R)-7-cyano-4-oxo-3,5-dihydro-2H-1,5-benzothiazepin-3-yl]carbamate (500 mg, 1.57 mmol, 1.0 eq) in analogy to general procedure 6 and was obtained as light yellow solid (600 mg, 1.7 mmol, 98% yield). MS (ESI): 353.3 [M+H]⁺

### Step b) [(Z)-[amino-[(3R)-3-(tert-butoxycarbonylamino)-4-oxo-3,5-dihydro-2H-1,5-benzothiazepin-7-yl]methylene]amino]3-(tert-butoxycarbonylamino)oxetane-3-carboxylate

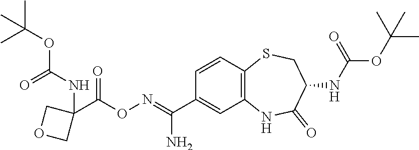 [SMILES: CC(C)(C)OC(=O)N[C@H]1CSc2ccc(/C(N)=N/OC(=O)C3(NC(=O)OC(C)(C)C)COC3)cc2NC1=O]

The title compound was prepared from tert-butyl N-[(3R)-7-[(Z)-N′-hydroxycarbamimidoyl]-4-oxo-3,5-dihydro-2H-1,5-benzothiazepin-3-yl]carbamate (550 mg, 1.56 mmol, 1.0 eq) and 3-((tert-butoxycarbonyl)amino)oxetane-3-carboxylic acid (508.52 mg, 2.34 mmol, 1.5 eq, CAS 1159736-25-0) in analogy to general procedure 8b and was obtained as yellow oil (1500 mg, 2.72 mmol, 65% yield). MS (ESI): 552.4 [M+H]⁺

### Step c) tert-butyl N-[(3R)-7-[5-[3-(tert-butoxycarbonylamino)oxetan-3-yl]-1,2,4-oxadiazol-3-yl]-4-oxo-3,5-dihydro-2H-1,5-benzothiazepin-3-yl]carbamate

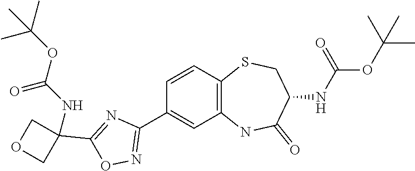 [SMILES: CC(C)(C)OC(=O)N[C@H]1CSc2ccc(-c3noc(C4(NC(=O)OC(C)(C)C)COC4)n3)cc2NC1=O]

The title compound was prepared from [(Z)-[amino-[(3R)-3-(tert-butoxycarbonylamino)-4-oxo-3,5-dihydro-2H-1,5-benzothiazepin-7-yl]methylene]amino]3-(tert-butoxycarbonylamino)oxetane-3-carboxylate (1.5 g, 2.72 mmol) in analogy to general procedure 9a and was obtained as white solid (550 mg, 38% yield). MS (ESI): 422.2 [M-2xisobutene+H]⁺

### Step d) tert-butyl N-[(3R)-7-[5-[3-(tert-butoxycarbonylamino)oxetan-3-yl]-1,2,4-oxadiazol-3-yl]-4-oxo-5-[[4-[5-(trifluoromethyl)-1,2,4-oxadiazol-3-yl]phenyl]methyl]-2,3-dihydro-1,5-benzothiazepin-3-yl]carbamate

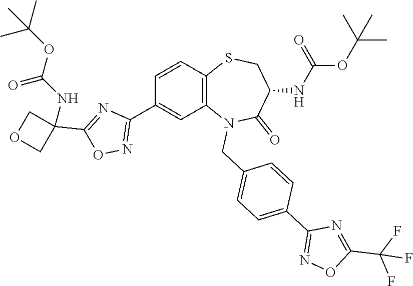 [SMILES: CC(C)(C)OC(=O)N[C@H]1CSc2ccc(-c3noc(C4(NC(=O)OC(C)(C)C)COC4)n3)cc2N(Cc2ccc(-c3noc(C(F)(F)F)n3)cc2)C1=O]

The title compound was prepared from tert-butyl N-[(3R)-7-[5-[3-(tert-butoxycarbonylamino)oxetan-3-yl]-1,2,4-oxadiazol-3-yl]-4-oxo-3,5-dihydro-2H-1,5-benzothiazepin-3-yl]carbamate (400 mg, 0.75 mmol, 1.0 eq) and 3-[4-(bromomethyl)phenyl]-5-(trifluoromethyl)-1,2,4-oxadiazole (276.22 mg, 0.9 mmol, 1.2 eq, CAS 2093101-98-3) in analogy to general procedure 1a and was obtained as white solid (550 mg, 59% yield). MS (ESI): 648.4 [M-2xisobutene+H]⁺

### Step e) tert-butyl N-[(3R)-7-[5-[3-(tert-butoxycarbonylamino)oxetan-3-yl]-1,2,4-oxadiazol-3-yl]-1,1,4-trioxo-5-[[4-[5-(trifluoromethyl)-1,2,4-oxadiazol-3-yl]phenyl]methyl]-2,3-dihydro-1lambda6,5-benzothiazepin-3-yl]carbamate

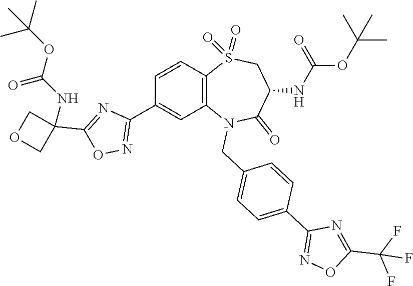 [SMILES: CC(C)(C)OC(=O)N[C@H]1CS(=O)(=O)c2ccc(-c3noc(C4(NC(=O)OC(C)(C)C)COC4)n3)cc2N(Cc2ccc(-c3noc(C(F)(F)F)n3)cc2)C1=O]

The title compound was prepared from tert-butyl N-[(3R)-7-[5-[3-(tert-butoxycarbonylamino)oxetan-3-yl]-1,2,4-oxadiazol-3-yl]-4-oxo-5-[[4-[5-(trifluoromethyl)-1,2,4-oxadiazol-3-yl]phenyl]methyl]-2,3-dihydro-1,5-benzothiazepin-3-yl]carbamate (500 mg, 0.66 mmol) in analogy to general procedure 10 and was obtained as white solid (110 mg, 20% yield). MS (ESI): 680.3 [M-2xisobutene+H]⁺

### Step f) (3R)-3-amino-7-[5-(3-aminooxetan-3-yl)-1,2,4-oxadiazol-3-yl]-1,1-dioxo-5-[[4-[5-(trifluoromethyl)-1,2,4-oxadiazol-3-yl]phenyl]methyl]-2,3-dihydro-1lambda6,5-benzothiazepin-4-one

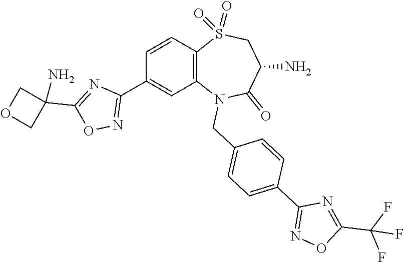 [SMILES: N[C@H]1CS(=O)(=O)c2ccc(-c3noc(C4(N)COC4)n3)cc2N(Cc2ccc(-c3noc(C(F)(F)F)n3)cc2)C1=O]

To a solution of tert-butyl N-[(3R)-7-[5-[3-(tert-butoxycarbonylamino)oxetan-3-yl]-1,2,4-oxadiazol-3-yl]-1,1,4-trioxo-5-[[4-[5-(trifluoromethyl)-1,2,4-oxadiazol-3-yl]phenyl]methyl]-2,3-dihydro-1λ⁶,5-benzothiazepin-3-yl]carbamate (100 mg, 0.13 mmol, 1.0 eq) in DCM (5 mL) was added trifluoroacetic acid (1.0 mL, 12.98 mmol, 103 eq) at RT and the mixture was stirred from 30 min. The pH was adjusted to around 9 by adding solid NaHCO₃. The mixture was poured into water (20 ml) and extracted with DCM (3×30 ml). The combined organic phase was washed with brine (30 ml), dried over anhydrous sodium sulfate, and concentrated under reduced pressure. The remaining crude was purified by reverse phase preparative HPLC to afford the title compound (11.7 mg, 0.02 mmol, 15% yield) as white solid. MS (ESI): 592.1 [M+H]⁺

### Example 85

### (3R)-3-amino-7-[5-(2-chloro-3-pyridyl)-1,2,4-oxadiazol-3-yl]-5-[[4-(5-cyclopropyl-1,2,4-oxadiazol-3-yl)phenyl]methyl]-1,1-dioxo-2,3-dihydro-1λ⁶,5-benzothiazepin-4-one

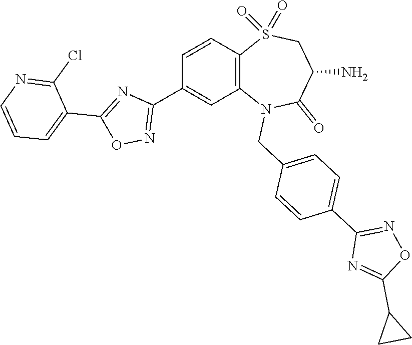 [SMILES: N[C@H]1CS(=O)(=O)c2ccc(-c3noc(-c4cccnc4Cl)n3)cc2N(Cc2ccc(-c3noc(C4CC4)n3)cc2)C1=O]

### Step a) tert-butyl N-[(3R)-7-cyano-5-[[4-(5-cyclopropyl-1,2,4-oxadiazol-3-yl)phenyl]methyl]-4-oxo-2,3-dihydro-1,5-benzothiazepin-3-yl]carbamate

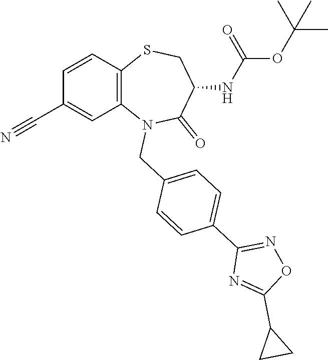 [SMILES: CC(C)(C)OC(=O)N[C@H]1CSc2ccc(C#N)cc2N(Cc2ccc(-c3noc(C4CC4)n3)cc2)C1=O]

The title compound was prepared in analogy to general procedure 1a from tert-butyl N-[(3R)-7-cyano-4-oxo-3,5-dihydro-2H-1,5-benzothiazepin-3-yl]carbamate(CAS: 2382087-69-4) (150 mg, 0.0.445 mmol, 1 eq) and Intermediate 16 and was obtained as yellow foam (823 mg, 42% yield). MS (ESI): 418.1 [M−Boc+H]⁺

### Step b) tert-butyl N-[(3R)-7-cyano-5-[[4-(5-cyclopropyl-1,2,4-oxadiazol-3-yl)phenyl]methyl]-1,1,4-trioxo-2,3-dihydro-1λ⁶, 5-benzothiazepin-3-yl]carbamate

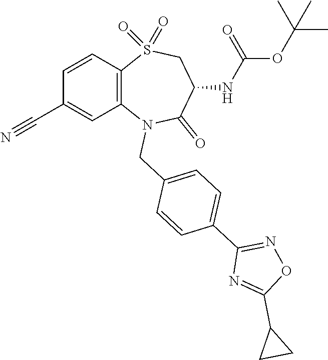 [SMILES: CC(C)(C)OC(=O)N[C@H]1CS(=O)(=O)c2ccc(C#N)cc2N(Cc2ccc(-c3noc(C4CC4)n3)cc2)C1=O]

The title compound was prepared in analogy to general procedure 10 from tert-butyl N-[(3R)-7-cyano-5-[[4-(5-cyclopropyl-1,2,4-oxadiazol-3-yl)phenyl]methyl]-4-oxo-2,3-dihydro-1,5-benzothiazepin-3-yl]carbamate (823 mg, 1.32 mmol) and was obtained as a light yellow solid (875 mg, 100% yield). MS (ESI): 494.1[M-isobutene+H]⁺

### Step c) tert-butyl N-[(3R)-5-[[4-(5-cyclopropyl-1,2,4-oxadiazol-3-yl)phenyl]methyl]-1,1,4-trioxo-7-[N′-hydroxycarbamimidoyl]-2,3-dihydro-1λ⁶, 5-benzothiazepin-3-yl]carbamate

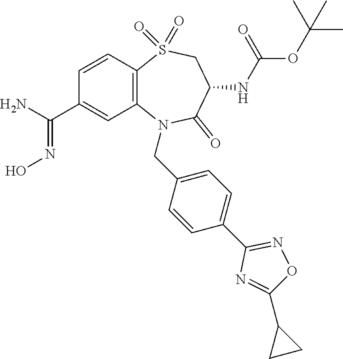 [SMILES: CC(C)(C)OC(=O)N[C@H]1CS(=O)(=O)c2ccc(/C(N)=N/O)cc2N(Cc2ccc(-c3noc(C4CC4)n3)cc2)C1=O]

The title compound was prepared in analogy to general procedure 6 from tert-butyl N-[(3R)-7-cyano-5-[[4-(5-cyclopropyl-1,2,4-oxadiazol-3-yl)phenyl]methyl]-1,1,4-trioxo-2,3-dihydro-1λ⁶,5-benzothiazepin-3-yl]carbamate (875 mg, 1.32 mmol) and was obtained as a light yellow foam (767 mg, 94% yield). MS (ESI): 583.3 [M+H]⁺

### Step d) [[amino-[(3R)-3-(tert-butoxycarbonylamino)-5-[[4-(5-cyclopropyl-1,2,4-oxadiazol-3-yl)phenyl]methyl]-1,1,4-trioxo-2,3-dihydro-1λ⁶, 5-benzothiazepin-7-yl]methylene]amino]2-chloropyridine-3-carboxylate

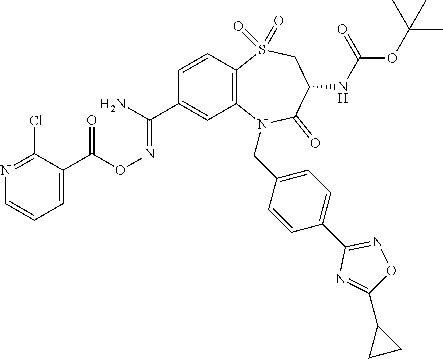 [SMILES: CC(C)(C)OC(=O)N[C@H]1CS(=O)(=O)c2ccc(/C(N)=N/OC(=O)c3cccnc3Cl)cc2N(Cc2ccc(-c3noc(C4CC4)n3)cc2)C1=O]

The title compound was prepared in analogy to general procedure 8a from tert-butyl N-[(3R)-5-[[4-(5-cyclopropyl-1,2,4-oxadiazol-3-yl)phenyl]methyl]-1,1,4-trioxo-7-[N′-hydroxycarbamimidoyl]-2,3-dihydro-1λ⁶,5-benzothiazepin-3-yl]carbamate (50 mg, 0.082 mmol, 1 eq) and 2-chloropyridine-3-carboxylic acid (13.1 mg, 0.09 mmol, 1.1 eq, CAS: 2942-59-8) and was obtained as white solid (51 mg, 83% yield). MS (ESI): 722.3 [M+H]⁺

### Step e) tert-butyl N-[(3R)-7-[5-(2-chloro-3-pyridyl)-1,2,4-oxadiazol-3-yl]-5-[[4-(5-cyclopropyl-1,2,4-oxadiazol-3-yl)phenyl]methyl]-1,1,4-trioxo-2,3-dihydro-1λ⁶, 5-benzothiazepin-3-yl]carbamate

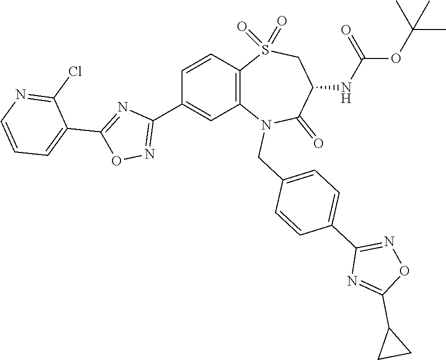 [SMILES: CC(C)(C)OC(=O)N[C@H]1CS(=O)(=O)c2ccc(-c3noc(-c4cccnc4Cl)n3)cc2N(Cc2ccc(-c3noc(C4CC4)n3)cc2)C1=O]

The title compound was prepared in analogy to general procedure 9a from [[amino-[(3R)-3-(tert-butoxycarbonylamino)-5-[[4-(5-cyclopropyl-1,2,4-oxadiazol-3-yl)phenyl]methyl]-1,1,4-trioxo-2,3-dihydro-1λ⁶,5-benzothiazepin-7-yl]methylene]amino]2-chloropyridine-3-carboxylate (51 mg, 0.071 mmol) and was obtained as a white solid (25 mg, 4200 yield). MS (ESI): 648.1 [M-isobutene+H]⁺

### Step f) (3R)-3-amino-7-[5-(2-chloro-3-pyridyl)-1,2,4-oxadiazol-3-yl]-5-[[4-(5-cyclopropyl-1,2,4-oxadiazol-3-yl)phenyl]methyl]-1,1-dioxo-2,3-dihydro-1λ⁶,5-benzothiazepin-4-one

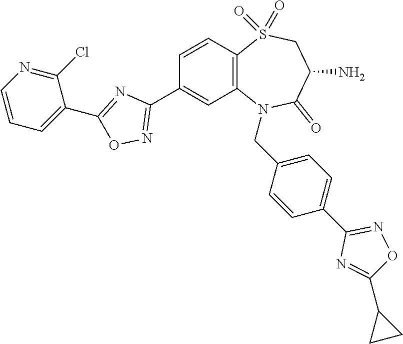 [SMILES: N[C@H]1CS(=O)(=O)c2ccc(-c3noc(-c4cccnc4Cl)n3)cc2N(Cc2ccc(-c3noc(C4CC4)n3)cc2)C1=O]

The title compound was prepared in analogy to general procedure 11a from tert-butyl N-I[(3R)-7-[5-(2-chloro-3-pyridyl)-1,2,4-oxadiazol-3-yl]-5-[[4-(5-cyclopropyl-1,2,4-oxadiazol-3-yl)phenyl]methyl]-1,1,4-trioxo-2,3-dihydro-1λ⁶,5-benzothiazepin-3-yl]carbamate (25 mg, 31.95 μmol) and was obtained as a white solid, as hydrochloride salt (21 mg, 100% yield). MS (ESI): 604.2 [M+H]⁺

### Examples 86 and 87 of the Following Table were Prepared in Analogy to Example 85 Using the Appropriate Carboxylic Acid Building Block

| | | Systematic | Building | MS, ESI: |
|---|---|---|---|---|
| Ex. | Structure | Name | Blocks | m/z |
| 86 | 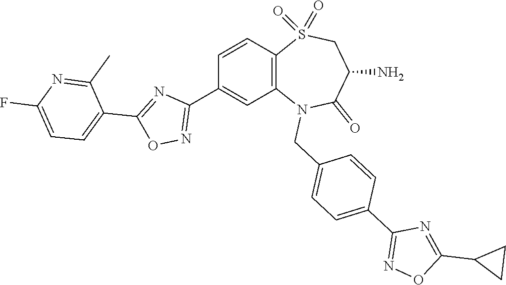 [SMILES: Cc1nc(F)ccc1-c1nc(-c2ccc3c(c2)N(Cc2ccc(-c4noc(C5CC5)n4)cc2)C(=O)[C@@H](N)CS3(=O)=O)no1] | (3R)-3-amino- 5-[[4-(5- cyclopropyl- 1,2,4- oxadiazol-3- yl)phenyl] methyl]-7-[5-(6- fluoro-2- methyl-3- pyridyl)-1,2,4- oxadiazol-3- yl]-1,1-dioxo- 2,3-dihydro- 1λ⁶,5- benzothiazepin- 4-one (*) | 6-fluoro- 2-methyl- pyridine- 3- carboxylic acid) (CAS: 884494- 97-7) | 602.3 [M + H]⁺ |
| 87 | 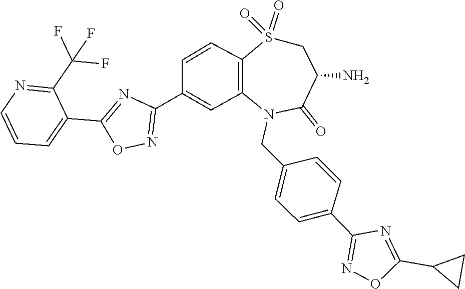 [SMILES: N[C@H]1CS(=O)(=O)c2ccc(-c3noc(-c4cccnc4C(F)(F)F)n3)cc2N(Cc2ccc(-c3noc(C4CC4)n3)cc2)C1=O] | (3R)-3-amino- 5-[[4-(5- cyclopropyl- 1,2,4- oxadiazol-3- yl)phenyl] methyl]-1,1- dioxo-7-[5-[2- (trifluoromethyl)- 3-pyridyl]- 1,2,4- oxadiazol-3- yl]-2,3-dihydro- 1λ⁶,5- benzothiazapin- 4-one (*) | 2- (trifluoro- methyl) pyridine-3- carboxylic acid (CAS: 131747- 43-8) | 638.4 [M + H]⁺ |

* as hydrochloride salt

### Intermediate 1

### [6-[4-[[tert-butyl(dimethyl)silyl]oxymethyl]phenyl]-3-pyridyl]methyl methanesulfonate

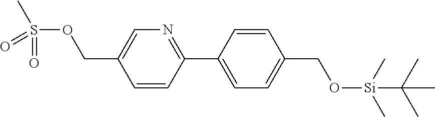 [SMILES: CC(C)(C)[Si](C)(C)OCc1ccc(-c2ccc(COS(C)(=O)=O)cn2)cc1]

### Step a) [6-[4-[[tert-butyl(dimethyl)silyl]oxymethyl]phenyl]-3-pyridyl]methanol

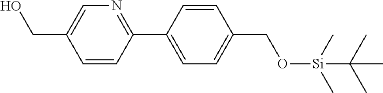 [SMILES: CC(C)(C)[Si](C)(C)OCc1ccc(-c2ccc(CO)cn2)cc1]

(6-bromo-3-pyridyl)methanol (100 mg, 0.532 mmol, 1.0 eq, CAS 122306-01-8) was combined with [4-[[tert-butyl(dimethyl)silyl]oxymethyl]phenyl]boronic acid (169.91 mg, 0.638 mmol, 1.2 eq, CAS 162356-89-0), 1,1-bis(diphenylphosphino)ferrocene dichloro palladium(II) CH₂Cl₂ adduct (43.43 mg, 0.053 mmol, 0.1 eq) and K₂CO₃ (147.02 mg, 1.06 mmol, 2.000 eq) in water (0.010 mL) and 1,4-dioxane (1 mL). The reaction was heated to 80° C. and was stirred for 6 h. The solution was directly concentrated and purified by flash column chromatography on silica gel (0-100% EtOAc in heptane) to afford [6-[4-[[tert-butyl(dimethyl)silyl]oxymethyl]phenyl]-3-pyridyl]methanol (75 mg, 35%) as light brown solid. MS (ESI): 330.2 [M+H]⁺

### Step b) [6-[4-[[tert-butyl(dimethyl)silyl]oxymethyl]phenyl]-3-pyridyl]methyl methanesulfonate

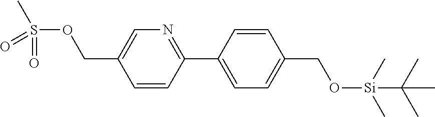 [SMILES: CC(C)(C)[Si](C)(C)OCc1ccc(-c2ccc(COS(C)(=O)=O)cn2)cc1]

[6-[4-[[tert-butyl(dimethyl)silyl]oxymethyl]phenyl]-3-pyridyl]methanol (71 mg, 0.18 mmol, 1.0 eq) was combined with triethylamine (24.33 uL, 0.18 mmol, 1.0 eq) in DCM (1 mL) and the solution was cooled down to 0° C. Then methanesulfonyl chloride (19.99 mg, 13.6 uL, 0.175 mmol, 1.0 eq) was added and the reaction allowed to warm to RT. After stirring for 2 h water was added and the mixture was extracted with DCM (3×). The organic layer was washed with brine, dried over magnesium sulfate, filtered and concentrated to afford [6-[4-[[tert-butyl(dimethyl)silyl]oxymethyl]phenyl]-3-pyridyl]methyl methanesulfonate (85 mg, 39%) as light brown solid. MS (ESI): 408.2 [M+H]⁺

### Intermediate 2

### 3-[5-(bromomethyl)-2-pyridyl]-5-(trifluoromethyl)-1,2,4-oxadiazole

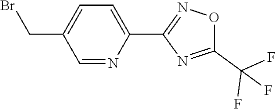 [SMILES: FC(F)(F)c1nc(-c2ccc(CBr)cn2)no1]

### Step a) 3-(5-methyl-2-pyridyl)-5-(trifluoromethyl)-1,2,4-oxadiazole

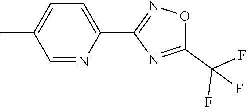 [SMILES: Cc1ccc(-c2noc(C(F)(F)F)n2)nc1]

To a solution of N′-hydroxy-5-methyl-pyridine-2-carboxamidine (500 mg, 3.31 mmol, 1.0 eq) in THF (15 mL) was added trifluoroacetic anhydride (0.71 mL, 5.03 mmol, 1.52 eq) at 0° C. and the mixture was stirred at RT for 16 h. The reaction mixture was diluted with water (10 mL) and extracted with EtOAc (10 mL×3). The combined organic layer was dried over anhydrous sodium sulfate, filtered and concentrated under reduced pressure to afford 3-(5-methyl-2-pyridyl)-5-(trifluoromethyl)-1,2,4-oxadiazole (480 mg, 2.09 mmol, 63% yield) as yellow oil. MS (ESI): 229.9 [M+H]⁺

### Step b) 3-[5-(bromomethyl)-2-pyridyl]-5-(trifluoromethyl)-1,2,4-oxadiazole

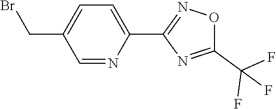 [SMILES: FC(F)(F)c1nc(-c2ccc(CBr)cn2)no1]

To a solution of 3-(5-methyl-2-pyridyl)-5-(trifluoromethyl)-1,2,4-oxadiazole (1.0 g, 4.36 mmol, 1.0 eq) and NBS (0.78 g, 4.36 mmol, 1.0 eq) in CCl₄ (20 mL) was added AIBN (0.01 g, 0.09 mmol, 0.02 eq) at RT and the mixture was heated to 80° C. and stirred for 16 h. After cooling to RT, the mixture was filtered and the filtrate was concentrated under reduced pressure. The remaining residue was purified by column chromatography on silica gel (0-10% EtOAc in petroleum ether). Before concentration of the obtained column fractions, 1N HCl in EtOAc (1.1 mL, 4.4 mmol, 1.01 eq) was added, followed by concentration under reduced pressure to afford 3-[5-(bromomethyl)-2-pyridyl]-5-(trifluoromethyl)-1,2,4-oxadiazole; (1000 mg, 2.9 mmol, 41% yield) as a yellow solid, as hydrochloride salt. MS (ESI): 308.1 [M+H]⁺

### Intermediate 3

### 2-[4-(bromomethyl)phenyl]-5-tert-butyl-1,3,4-oxadiazole

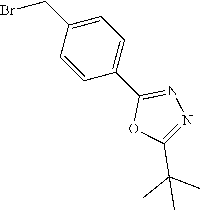 [SMILES: CC(C)(C)c1nnc(-c2ccc(CBr)cc2)o1]

### Step a) N′-(2,2-dimethylpropanoyl)-4-methyl-benzohydrazide

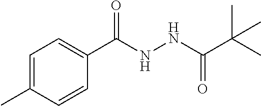 [SMILES: Cc1ccc(C(=O)NNC(=O)C(C)(C)C)cc1]

The title compound was prepared from p-toluic acid (2000 mg, 14.7 mmol, 1.0 eq) and pivalic acid hydrazide (2047 mg, 17.6 mmol, 1.2 eq) in analogy to general procedure 3 and was obtained as white solid (2200 mg, 9.39 mmol, 62% yield). MS (ESI): 235.3 [M+H]⁺.

### Step b) 2-tert-butyl-5-(p-tolyl)-1,3,4-oxadiazole

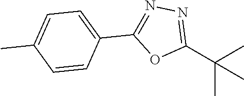 [SMILES: Cc1ccc(-c2nnc(C(C)(C)C)o2)cc1]

The title compound was prepared from N′-(2,2-dimethylpropanoyl)-4-methyl-benzohydrazide (1050 mg, 4.48 mmol) in analogy to general procedure 5a and was obtained as light yellow solid (1100 mg, 5.09 mmol, 95% yield). MS (ESI): 217.1 [M+H]⁺

### Step c) 2-[4-(bromomethyl)phenyl]-5-tert-butyl-1,3,4-oxadiazole

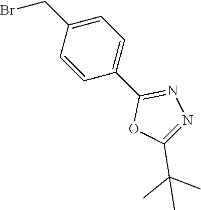 [SMILES: CC(C)(C)c1nnc(-c2ccc(CBr)cc2)o1]

To a solution 2-tert-butyl-5-(p-tolyl)-1,3,4-oxadiazole (289 mg, 1.34 mmol, 1.0 eq) in acetonitrile (5.3 mL) was added N-bromosuccinimide (285.4 mg, 1.6 mmol, 1.2 eq) and 2,2′-azobis(2-methylpropionitrile) (65.8 mg, 0.4 mmol, 0.3 eq). The mixture was stirred at 80° C. for 3 hours. The reaction was poured into brine and extracted 3× with EtOAc. The combined organic phases were dried over sodium sulfate, filtered and concentrated. The remaining crude was purified using flash column chromatography on silica gel (0-35% EtOAc in heptane) to afford 2-[4-(bromomethyl)phenyl]-5-tert-butyl-1,3,4-oxadiazole (177 mg, 40%) as light blue solid. MS (ESI): 295.0 [M+H]⁺

### Intermediate 4

### 2-[4-(bromomethyl)phenyl]-5-(trifluoromethyl)-1,3,4-oxadiazole

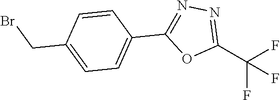 [SMILES: FC(F)(F)c1nnc(-c2ccc(CBr)cc2)o1]

To a solution of 2-(4-methylphenyl)-5-(trifluoromethyl)-1,3,4-oxadiazole (250 mg, 1.1 mmol, 1 eq, CAS 1352872-11-7) and NBS (156.01 mg, 0.88 mmol, 0.8 eq) in CCl₄ (5 mL) was added AIBN (89.96 mg, 0.55 mmol, 0.5 eq) at 25° C. under nitrogen atmosphere and the mixture was stirred at 80° C. for 12 h. The reaction mixture was poured into water (10 mL) and extracted with DCM (3×10 mL). The combined organic extracts were washed with brine (5 mL), dried over sodium sulfate, filtered, and concentrated to give 2-[4-(bromomethyl)phenyl]-5-(trifluoromethyl)-1,3,4-oxadiazole (400 mg, 1.3 mmol, 71% yield) as light yellow solid. MS (ESI): 307.0 [M+H]⁺.

The intermediates of the following table were prepared in analogy to Example Intermediate 4, using the indicated building block

| | | | | MS, |
|---|---|---|---|---|
| | | | | ESI: |
| Intermediate | Structure | Systematic Name | Building Block | m/z |
| 5 | 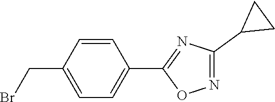 [SMILES: BrCc1ccc(-c2nc(C3CC3)no2)cc1] | 5-[4- (bromomethyl)phenyl]- 3-cyclopropyl-1,2,4- oxadiazole | 3-cyclopropyl-5-(4- methylphenyl)-1,2,4- oxadiazole (CAS 2386184-90-1) | 278.9 [M + H]⁺ |
| 6 | 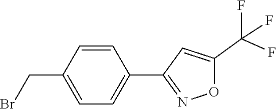 [SMILES: FC(F)(F)c1cc(-c2ccc(CBr)cc2)no1] | 3-[4- (bromomethyl)phenyl]- 5- (trifluoromethyl) isoxazole | 3-(4-Methylphenyl)-5- (trifluoromethyl) isoxazole (CAS 187812-15-3) | 308.1 [M + H]⁺ |

### Intermediate 7

### 5-[4-(bromomethyl)phenyl]-3-(trifluoromethyl)-1,2,4-oxadiazole

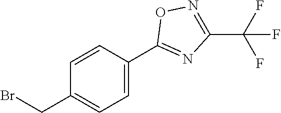 [SMILES: FC(F)(F)c1noc(-c2ccc(CBr)cc2)n1]

### Step a) [(Z)-[amino(p-tolyl)methylene]amino]2,2,2-trifluoroacetate

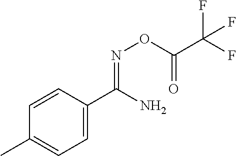 [SMILES: Cc1ccc(/C(N)=N/OC(=O)C(F)(F)F)cc1]

To a solution of 2,2,2-trifluoro-N′-hydroxy-acetamidine (150 mg, 1.17 mmol, 1.0 eq) and DIEA (379 mg, 2.93 mmol, 2.5 eq) in DCM (3 mL) was added dropwise a solution of 4-methylbenzoyl chloride (190 mg, 1.23 mmol, 1.05 eq) in DCM (2 mL) at 0° C. After full addition, the mixture was allowed to warm to RT and stirred for 1 h. The reaction mixture was directly concentrated under reduced pressure. The remaining residue was dissolved in EtOAc (20 mL) and water (10 mL) was added and the layers separated. The aqueous phase was extracted with EtOAc (10 mL×2). The combined organic phase was washed with brine (10 mL), dried over anhydrous sodium sulfate, filtered, and concentrated under vacuum to give [(Z)-(1-amino-2,2,2-trifluoro-ethylidene)amino]4-methylbenzoate (350 mg, 1.42 mmol, 102% yield) as off-white solid. MS (ESI): 247.1 [M+H]⁺

### Step b) 5-(p-tolyl)-3-(trifluoromethyl)-1,2,4-oxadiazole

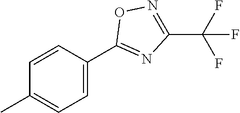 [SMILES: Cc1ccc(-c2nc(C(F)(F)F)no2)cc1]

To a solution of [(Z)-(1-amino-2,2,2-trifluoro-ethylidene)amino]4-methylbenzoate (350 mg, 1.42 mmol, 1.0 eq) in DMSO (4 mL) was added KOH (160 mg, 2.84 mmol, 2.0 eq) at RT and stirred for 1 h. The reaction mixture was poured into water (20 mL) and extracted with EtOAc (10 mL×3). The combined organic phase was washed with brine (10 mL), dried over anhydrous sulfate, filtered, and concentrated under reduced pressure. The remaining residue was purified by preparative TLC (20% EtOAc in petroleum ether) to afford 5-(p-tolyl)-3-(trifluoromethyl)-1,2,4-oxadiazole (190 mg, 0.83 mmol, 56% yield) as light yellow solid. MS (ESI): 229.1 [M+H]⁺

### Step c) 5-[4-(bromomethyl)phenyl]-3-(trifluoromethyl)-1,2,4-oxadiazole

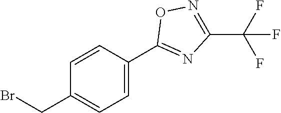 [SMILES: FC(F)(F)c1noc(-c2ccc(CBr)cc2)n1]

To a solution of 3-cyclopropyl-5-(p-tolyl)-1,2,4-oxadiazole (500 mg, 2.5 mmol, 1.0 eq) and NBS (533 mg, 3.0 mmol, 1.2 eq) in CCl₄ (5 mL) was added AIBN (82 mg, 0.5 mmol, 0.2 eq) and the mixture was heated to 90° C. for 12 h under inert atmosphere. After cooling to RT, the mixture was filtered and concentrated under reduced pressure to afford 5-[4-(bromomethyl)phenyl]-3-(trifluoromethyl)-1,2,4-oxadiazole (300 mg, 0.98 mmol, 74% yield) as light yellow oil. MS (ESI): 307.0 [M+H]⁺

### Intermediate 8

### 1-[4-(chloromethyl)phenyl]-4-(trifluoromethyl)imidazole

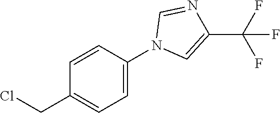 [SMILES: FC(F)(F)c1cn(-c2ccc(CCl)cc2)cn1]

### Step a) methyl 4-[4-(trifluoromethyl)imidazol-1-yl]benzoate

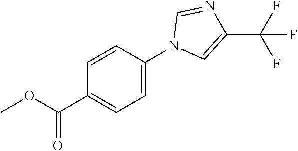 [SMILES: COC(=O)c1ccc(-n2cnc(C(F)(F)F)c2)cc1]

To a solution of 4-(trifluoromethyl)-1H-imidazole (1000 mg, 7.35 mmol, 1.0 eq) and methyl 4-fluorobenzoate (1359 mg, 8.82 mmol, 1.2 eq) in DMF (1 mL) was added potassium carbonate (2031 mg, 14.7 mmol, 2.0 eq) and the mixture was stirred at 100° C. for 12 h. The reaction was poured into water (60 mL). The aqueous phase was extracted with EtOAc (50 mL×3). The combined organic phase was washed with brine (100 mL×2), dried with anhydrous sodium sulfate, filtered and concentrated in vacuum. The remaining residue was purified by column chromatography on silica gel (0-20% EtOAc in petroleum ether) to afford methyl 4-[4-(trifluoromethyl)imidazol-1-yl]benzoate (1100 mg, 4.07 mmol, 55% yield) as white solid. MS (ESI): 271.0 [M+H]⁺

### Step b) [4-[4-(trifluoromethyl)imidazol-1-yl]phenyl]methanol

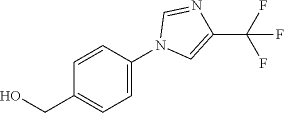 [SMILES: OCc1ccc(-n2cnc(C(F)(F)F)c2)cc1]

To a solution of methyl 4-[4-(trifluoromethyl)imidazol-1-yl]benzoate (500 mg, 1.85 mmol, 1.0 eq) in THF (10 mL) was added diisobutylaluminium hydride (5.55 mL, 5.55 mmol, 3.0 eq) at 0° C. The mixture was allowed to warm to RT and stirred for 1 h. The reaction was quenched with water (0.24 mL), 15% NaOH (aq. 0.24 mL) and water (0.72 mL) sequentially, dried with anhydrous sodium sulfate, filtered and concentrated in vacuum to afford [4-[4-(trifluoromethyl)imidazol-1-yl]phenyl]methanol (280.0 mg, 1.16 mmol, 63% yield) as yellow solid. MS (ESI): 243.1 [M+H]⁺

### Step c) 1-[4-(chloromethyl)phenyl]-4-(trifluoromethyl)imidazole

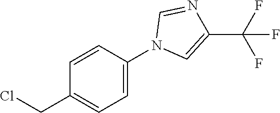 [SMILES: FC(F)(F)c1cn(-c2ccc(CCl)cc2)cn1]

To a solution of [4-[4-(trifluoromethyl)imidazol-1-yl]phenyl]methanol (100.0 mg, 0.41 mmol, 1.0 eq) in DCM (5 mL) was added thionyl chloride (0.09 mL, 1.24 mmol, 3.0 eq) slowly via syringe at RT and the solution was stirred for 1 h. The solution was concentrated directly to give 1-[4-(chloromethyl)phenyl]-4-(trifluoromethyl)imidazole (90 mg, 0.35 mmol, 83% yield) as a yellow oil. MS (ESI): 261.1 [M+H]⁺

### Intermediate 9

### 5-[4-(bromomethyl)phenyl]-3-(trifluoromethyl)isoxazole

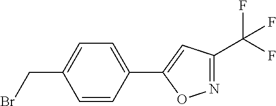 [SMILES: FC(F)(F)c1cc(-c2ccc(CBr)cc2)on1]

### Step a) 5-(p-tolyl)-3-(trifluoromethyl)isoxazole

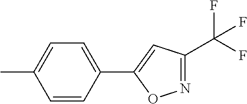 [SMILES: Cc1ccc(-c2cc(C(F)(F)F)no2)cc1]

To a solution of 4-ethynyltoluene (1.0 g, 8.61 mmol, 1.0 eq) in chloroform (20 mL) was successively added acetic acid (57 uL), 2,2,2-trifluoroethylamine (2.56 g, 25.83 mmol, 3.0 eq) and isoamyl nitrite (3.47 mL, 25.83 mmol, 3.0 eq) under inert atmosphere. Then CuI (166.7 mg, 0.88 mmol, 0.1 eq) and ZnBr₂ (3.88 g, 17.22 mmol, 2.0 eq) were added at RT and stirred for 24 h. The solution was poured into water and extracted with EtOAc (3×). The combined organic phase was washed with brine, dried over anhydrous sodium sulfate and concentrated under reduced pressure. The remaining crude was purified by column chromatography on silica gel (100% petroleum ether) to afford the tile compound as a yellow solid (1.1 g, 33%). MS (ESI): 228.0 [M+H]⁺

### Step b) 5-[4-(bromomethyl)phenyl]-3-(trifluoromethyl)isoxazole

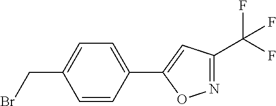 [SMILES: FC(F)(F)c1cc(-c2ccc(CBr)cc2)on1]

The title compound was prepared from 5-(p-tolyl)-3-(trifluoromethyl)isoxazole (150.0 mg, 0.66 mmol) in analogy to general procedure 12 and was obtained as white solid (50 mg, 25%). ¹H-NMR (CDCl₃, 400 MHz): 7.85-7.79 (m, 2H), 7.55 (d, J=8.1 Hz, 2H), 6.77 (s, 1H), 4.53 (s, 2H).

### Intermediate 10

### 3-[4-(bromomethyl)phenyl]-5-(2,2,2-trifluoroethyl)-1,2,4-oxadiazole

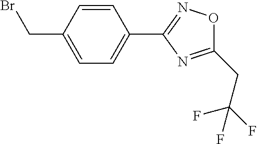 [SMILES: FC(F)(F)Cc1nc(-c2ccc(CBr)cc2)no1]

### Step a) [(Z)-[amino(p-tolyl)methylene]amino]3,3,3-trifluoropropanoate

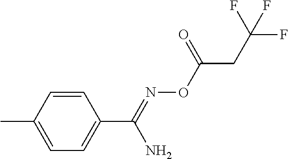 [SMILES: Cc1ccc(/C(N)=N/OC(=O)CC(F)(F)F)cc1]

To a solution of N′-hydroxy-4-methyl-benzamidine (300 mg, 1.88 mmol, 1.0 eq) in THF (9.39 mL) was added HATU (1.07 g, 2.82 mmol, 1.5 eq), DIEA (606 mg, 820 μL, 4.69 mmol, 2.5 eq) and 3,3,3-trifluoropropionic acid (288 mg, 199 μL, 2.25 mmol, 1.2 eq). The yellow solution was stirred at RT for 1 h. The reaction was diluted with EtOAc and brine. The phases were separated and the aqueous phase was washed twice with EtOAc. The combined organic phases were dried over sodium sulfate, filtered and concentrated under reduced pressure. The remaining crude was purified by column chromatography on silica gel (0 to 10% MeOH in DCM) to afford the title compound (310 mg, 64%) as white solid. MS (ESI): 261.1 [M+H]⁺

### Step b) 3-(p-tolyl)-5-(2,2,2-trifluoroethyl)-1,2,4-oxadiazole

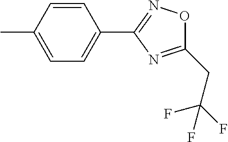 [SMILES: Cc1ccc(-c2noc(CC(F)(F)F)n2)cc1]

[(Z)-[amino(p-tolyl)methylene]amino]3,3,3-trifluoropropanoate (310 mg, 1.19 mmol, 1.0 eq) was suspended in toluene. The mixture was stirred at 110° C. for 6 h. The reaction mixture was then directly purified by column chromatography on silica gel (0-5% MeOH in DCM) to afford the title compound (246 mg, 80%) as white solid. MS (ESI): 243.1 [M+H]⁺

### Step c) 3-[4-(bromomethyl)phenyl]-5-(2,2,2-trifluoroethyl)-1,2,4-oxadiazole

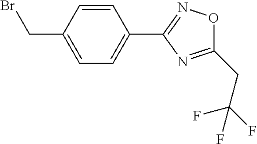 [SMILES: FC(F)(F)Cc1nc(-c2ccc(CBr)cc2)no1]

To a solution 3-(p-tolyl)-5-(2,2,2-trifluoroethyl)-1,2,4-oxadiazole (100 mg, 0.41 mmol, 1.0 eq) in acetonitrile (4.13 mL) was added NBS (77.16 mg, 0.43 mmol, 1.05 eq) and 2,2′-azobis(2-methylpropionitrile) (10.17 mg, 0.062 mmol, 0.15 eq). The mixture was stirred at 80° C. for 3 h. The reaction was poured into brine and extracted EtOAc (3×). The combined organic phases were dried over sodium sulfate, filtered and concentrated under reduced pressure. The remaining crude was purified using column chromatography on silica gel (0-75% DCM in heptane) to afford 3-[4-(bromomethyl)phenyl]-5-(2,2,2-trifluoroethyl)-1,2,4-oxadiazole (22.4 mg, 16%) as white solid. MS (ESI): 321.0 [M+H]⁺

### Intermediate 11

### 2-[4-(bromomethyl)phenyl]-5-(difluoromethyl)pyridine

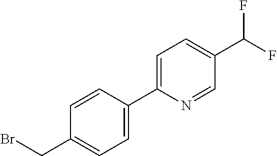 [SMILES: FC(F)c1ccc(-c2ccc(CBr)cc2)nc1]

### Step a) 5-(difluoromethyl)-2-(p-tolyl)pyridine

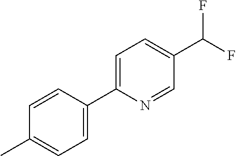 [SMILES: Cc1ccc(-c2ccc(C(F)F)cn2)cc1]

A mixture of p-tolylboronic acid (255 mg, 1.88 mmol, 1.0 eq), 2-chloro-5-(difluoromethyl)pyridine (368 mg, 2.25 mmol, 1.2 eq) and K₂CO₃ (778 mg, 5.63 mmol, 3.0 eq) in 1,4-dioxane (6.63 mL) and water (663.43 uL) was degassed with argon for 5 min. 1.1-bis(diphenylphosphino)ferrocene dichloro palladium(II) (27.45 mg, 37.51 μmol, 0.02 eq) was added at RT. The reaction mixture was heated to 80° C. and stirred for 3.5 h. The reaction mixture was partitioned between EtOAc (150 ml) and water (75 ml). The layers were separated and the aqueous layer extracted with one 50 ml portion of EtOAc. The combined organic layers were washed with one 150 ml portion of brine, dried over MgSO₄ and concentrated under reduced pressure. The crude material was purified by flash chromatography on silica gel (0-20% EtOAc in heptane) to afford 5-(difluoromethyl)-2-(p-tolyl)pyridine (325 mg, 78%) as white solid. MS (ESI): 220.1 [M+H]⁺

### Step b) 2-[4-(bromomethyl)phenyl]-5-(difluoromethyl)pyridine

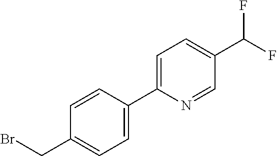 [SMILES: FC(F)c1ccc(-c2ccc(CBr)cc2)nc1]

To a mixture of 5-(difluoromethyl)-2-(p-tolyl)pyridine (3.49 g, 15.9 mmol, 1.0 eq) in acetonitrile (64 mL) was added N-bromosuccinimide (2.97 g, 16.7 mmol, 1.05 eq) and 2,2′-azobis(2-methylpropionitrile) (784.2 mg, 4.78 mmol, 0.3 eq) at RT. The mixture was stirred at 80° C. for 3 h. The reaction was poured into brine (100 ml) and extracted with three portions of EtOAc (100 ml). The combined organic phase was dried over anhydrous sodium sulfate, filtered and concentrated under reduced pressure. The remaining crude material was purified by flash column chromatography on silica gel (0-10% EtOAc in heptane) to afford 2-[4-(bromomethyl)phenyl]-5-(difluoromethyl)pyridine (325 mg, 70%) as white solid. MS (ESI): 299.9 [M+H]⁺

### Intermediate 12

### 3-[4-(bromomethyl)phenyl]-5-(3,3-difluorocyclopentyl)-1,2,4-oxadiazole

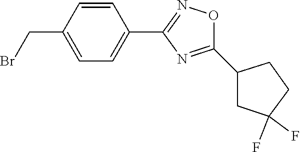 [SMILES: FC1(F)CCC(c2nc(-c3ccc(CBr)cc3)no2)C1]

### Step a) [(Z)-[amino(p-tolyl)methylene]amino]3,3-difluorocyclopentanecarboxylate

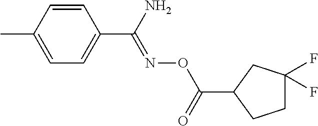 [SMILES: Cc1ccc(/C(N)=N/OC(=O)C2CCC(F)(F)C2)cc1]

To a solution of N′-hydroxy-4-methyl-benzamidine (300 mg, 1.88 mmol, 1.0 eq) in THF (9.39 mL) was added HATU (1.07 g, 2.82 mmol, 1.5 eq), DIEA (607 mg, 819.9 uL, 4.69 mmol, 2.5 eq) and 3,3-difluorocyclopentanecarboxylic acid (338.26 mg, 260.2 uL, 2.25 mmol, 1.2 eq). The yellow solution was stirred at RT for 2 h. The reaction was diluted with EtOAc and brine. The phases were separated and the aqueous phase washed twice with EtOAc. The combined organic phases were dried over sodium sulfate, filtered and concentrated under reduced pressure. The remaining crude was purified by column chromatography on silica gel (5-50% EtOAc in heptane) to afford the title compound as white sold (470 mg, 89%). MS (ESI): 283.1 [M+H]⁺

### Step b) 5-(3,3-difluorocyclopentyl)-3-(p-tolyl)-1,2,4-oxadiazole

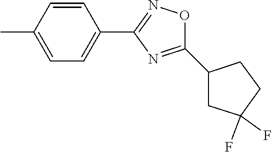 [SMILES: Cc1ccc(-c2noc(C3CCC(F)(F)C3)n2)cc1]

[(Z)-[amino(p-tolyl)methylene]amino]3,3-difluorocyclopentanecarboxylate (470 mg, 1.66 mmol, 1.0 eq) was suspended in toluene and heated to reflux for 6 h. The solution was directly purified by column chromatography on silica gel (0-5% MeOH in DCM) to afford the title compound as colorless liquid (392 mg, 89%). MS (ESI): 265.1 [M+H]⁺

### Step c) 3-[4-(bromomethyl)phenyl]-5-(3,3-difluorocyclopentyl)-1,2,4-oxadiazole

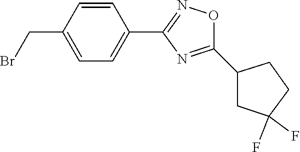 [SMILES: FC1(F)CCC(c2nc(-c3ccc(CBr)cc3)no2)C1]

To a solution of 5-(3,3-difluorocyclopentyl)-3-(p-tolyl)-1,2,4-oxadiazole (50 mg, 0.189 mmol, 1.0 eq) in acetonitrile (1.89 mL) was added N-bromosuccinimide (34 mg, 0.191 mmol, 1.01 eq) and 2,2′-azobis(2-methylpropionitrile) (4.66 mg, 0.028 mmol, 0.15 eq) and the mixture was stirred at 80° C. for 4 h. The reaction was quenched upon addition of sat. aqueous sodium thiosulfate and the mixture extracted with EtOAc (3×). The combined organic phases were dried over sodium sulfate, filtered and concentrated under reduced pressure. The remaining crude was purified using column chromatography on silica gel (5-75% DCM in heptane) to afford the title compound (29.6 mg, 46%) as white powder. MS (ESI): 343.1 [M+H]⁺

### Intermediate 13

### 3-[4-(bromomethyl)phenyl]-5-tert-butyl-1,2,4-oxadiazole

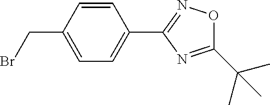 [SMILES: CC(C)(C)c1nc(-c2ccc(CBr)cc2)no1]

### Step a) 5-tert-butyl-3-(p-tolyl)-1,2,4-oxadiazole

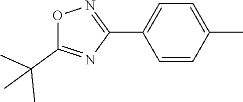 [SMILES: Cc1ccc(-c2noc(C(C)(C)C)n2)cc1]

A solution of N′-hydroxy-4-methyl-benzamidine (500 mg, 3.33 mmol, 1.0 eq. CAS 19227-13-5) and DIPEA (1.77 mL, 9.99 mmol, 3.0 eq) in toluene (10 mL) was added pivaloyl chloride (481.74 mg, 4.0 mmol, 1.2 eq. CAS 3282-30-2) at 0° C. After complete addition, the mixture was stirred at RT for 10 min. Then the temperature was increased to 80° C. and stirring continued for 16 h. The mixture was cooled and concentrated under reduced pressure. The residue was poured directly into water (10 mL). The aqueous phase was extracted with EtOAc (3×10 mL). The combined organic phase was washed with brine (25 mL), dried over anhydrous sodium sulfate, filtered and concentrated under reduced pressure. The residue was purified by column chromatography on silica gel (2-30% EtOAc in petroleum ether) to afford the title compound as colorless oil (814 mg, 3.76 mmol, 113% yield). MS (ESI): 217.0 [M+H]⁺

### Step b) 3-[4-(bromomethyl)phenyl]-5-tert-butyl-1,2,4-oxadiazole

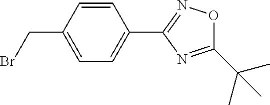 [SMILES: CC(C)(C)c1nc(-c2ccc(CBr)cc2)no1]

To a solution of 5-tert-butyl-3-(p-tolyl)-1,2,4-oxadiazole (300 mg, 1.39 mmol, 1.0 eq) in carbon tetrachloride (6 mL) was added AIBN (66.46 mg, 0.4 mmol, 0.29 eq), N-bromosuccinimide (296.3 mg, 1.66 mmol, 1.2 eq). Then the mixture was stirred at 80° C. for 2 h. The solution was poured into water (10 mL) and extracted with EtOAc (3×10 ml). The combined organic phase was washed with brine (15 mL), dried over sodium sulfate, filtered and concentrated in vacuo. The residue was purified by column chromatography on silica gel (0-5% EtOAc in heptane) to afford the title compound as a colorless oil (380 mg, 1.29 mmol, 93% yield).

MS (ESI): 295.1 [M+H]⁺

### Intermediate 14

### 2-[4-(bromomethyl)phenyl]-5-cyclopropyl-1,3,4-oxadiazole

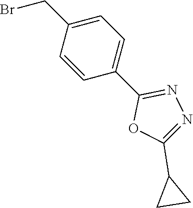 [SMILES: BrCc1ccc(-c2nnc(C3CC3)o2)cc1]

### Step a) 2-cyclopropyl-5-(p-tolyl)-1,3,4-oxadiazole

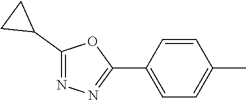

Cyclopropanecarboxylic acid (CAS: 1759-53-1)(300.95 mg, 278.7 uL, 3.5 mmol, 1.05 eq) was dissolved in THF(15 mL) and cooled to 0-5° C. Then CDI (566.8 mg, 3.4958 mmol, 1.05 eq) was added in one portion and the mixture was stirred for 90 min. To this mixture was then added a solution of 4-methylbenzohydrazide (CAS: 3619-22-5) (500 mg, 3.33 mmol, 1.0 eq) in THF (11 mL) via syringe and the mixture was stirred for 5 h. The reaction mixture was directly concentrated under reduced pressure and purified by column chromatography on silica gel (10-100% EtOAc in heptane) to afford the crude hydrazide intermediate (750 mg). This crude product was then suspended in toluene (11 mL) and Burgess Reagent (1.59 g, 6.66 mmol, 2.0 eq) was added. The resulting mixture was heated and stirred at 100° C. for 30 min. The reaction mixture was directly purified by column chromatography on silica gel (loaded as solution in toluene, 0-30% EtOAc in heptane) to afford 2-cyclopropyl-5-(p-tolyl)-1,3,4-oxadiazole (303 mg, 45%) as colorless oil. MS (ESI): 201.1 [M+H]⁺

### Step b) 2-[4-(bromomethyl)phenyl]-5-cyclopropyl-1,3,4-oxadiazole

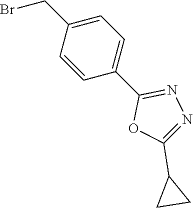 [SMILES: BrCc1ccc(-c2nnc(C3CC3)o2)cc1]

The title compound was prepared in analogy to general procedure 12 from 2-cyclopropyl-5-(p-tolyl)-1,3,4-oxadiazole (300 mg, 1.47 mmol, 1 eq) and was obtained as a white solid (240 mg, 55% yield).

MS (ESI): 279.1 [M+H]⁺.

### Intermediate 15

### 3-[4-(bromomethyl)phenyl]-1-cyclopropyl-1,2,4-triazole

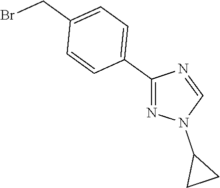 [SMILES: BrCc1ccc(-c2ncn(C3CC3)n2)cc1]

### Step a) N′-cyclopropyl-4-methyl-benzohydrazide

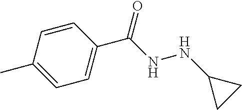

The solution of p-toluic acid (CAS: 99-94-5)(2.0 g, 14.69 mmol, 1.0 eq), HATU (11.0 g, 28.93 mmol, 1.97 eq) and N,N-diisopropylethylamine (10.23 mL, 58.76 mmol, 4.0 eq) in THF (30 mL) at RT was added cyclopropylhydrazine dihydrochloride (CAS: 213764-25-1 2.34 g, 16.16 mmol, 1.1 eq). The solution was stirred at RT for 2 h. The resulting mixture was poured into water (50 mL) and extracted with EtOAc (3×). The combined organic phases was washed with brine, dried over sodium sulfate and concentrated under reduced pressure. The crude material was purified by reversed-phase chromatography (Flash Column Welch Ultimate XB_C18 20-40 μm; 120 A, 60% MeCN in water). The resulting solution was extracted with EtOAc (300 mL). The organic phase was washed with brine (100 mL), dried over sodium sulfate, filtered and concentrated under reduced pressure to afford the title compound (600 mg, 3.15 mmol, 21% yield) as white solid. MS (ESI): 191.2 [M+H]⁺.

### Step b) 1-cyclopropyl-3-(p-tolyl)-1,2,4-triazole

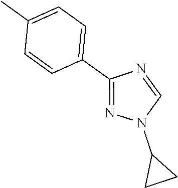

A solution of N′-cyclopropyl-4-methyl-benzohydrazide (500 mg, 2.63 mmol, 1.0 eq), ammonium acetate (1.5 g, 19.46 mmol, 7.4 eq) in trimethyl orthoformate (10.0 mL, 87.73 mmol, 33.38 eq) was stirred at 100° C. for 2 h. The mixture was poured into water (20 mL) and extracted with EtOAc (3×). The organic phase was washed with brine (50 mL), dried over sodium sulfate and concentrated under reduced pressure. The crude product was purified by column chromatography on silica gel (5-20% EtOAc in petroleum ether) to afford the title compound (120 mg, 0.6 mmol, 21% yield) as a light yellow oil. MS (ESI): 200.0 [M+H]⁺.

### Step c) 3-[4-(bromomethyl)phenyl]-1-cyclopropyl-1,2,4-triazole

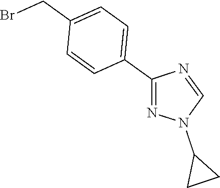 [SMILES: BrCc1ccc(-c2ncn(C3CC3)n2)cc1]

The title compound was prepared in analogy to general procedure 12 from 1-cyclopropyl-3-(p-tolyl)-1,2,4-triazole (80 mg, 0.4 mmol) and was obtained as a light yellow oil (75 mg, 43% yield). MS (ESI): 279.1 [M+H]⁺.

### Intermediate 16

### 3-[4-(bromomethyl)phenyl]-5-cyclopropyl-1,2,4-oxadiazole

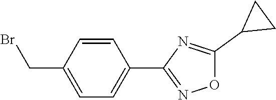 [SMILES: BrCc1ccc(-c2noc(C3CC3)n2)cc1]

### Step a) 5-cyclopropyl-3-(p-tolyl)-1,2,4-oxadiazole

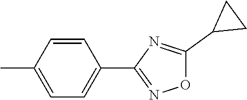

To a solution of N′-hydroxy-4-methyl-benzamidine (1.5 g, 9.49 mmol, 1.000 eq) in DMSO (10 mL) was added methyl cyclopropanecarboxylate (1.43 g, 1.44 mL, 14.23 mmol, 1.5 eq) at 0° C. Freshly powdered NaOH (569. mg, 14.23 mmol, 1.5 eq) was added and the mixture was stirred at RT overnight. Water was added and the mixture extracted three times with DCM. The combined organic layers were dried over MgSO₄, filtered, and the solvent removed under reduced pressure. The remaining crude material was purified by column chromatography on silica gel (0-30% EtOAc in heptane) to afford 5-cyclopropyl-3-(p-tolyl)-1,2,4-oxadiazole (1.75 g, 89%) as colourless liquid. MS (ESI): 201.0 [M+H]⁺

### Step b) 3-[4-(bromomethyl)phenyl]-5-cyclopropyl-1,2,4-oxadiazole

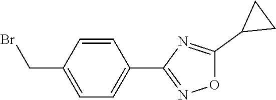 [SMILES: BrCc1ccc(-c2noc(C3CC3)n2)cc1]

To a solution of 5-cyclopropyl-3-(p-tolyl)-1,2,4-oxadiazole (1.74 g, 8.17 mmol, 1.0 eq) in MeCN (35 mL) was added N-bromosuccinimide (1.53 g, 8.58 mmol, 1.05 eq) and 2,2′-azobis(2-methylpropionitrile) (402 mg, 2.45 mmol, 0.3 eq). The mixture was stirred at 80° C. overnight. The reaction was poured into brine and extracted 3× with EtOAc. The combined organic phases were dried over sodium sulfate, filtered and concentrated. The remaining crude was purified using column chromatography on silica gel (0-20% EtOAc in heptane) to afford 3-[4-(bromomethyl)phenyl]-5-cyclopropyl-1,2,4-oxadiazole (2.11 g, 80%) as white crystalline solid. MS (ESI): 279.0 [M+H]⁺

### Intermediate 17

### 2-[4-(bromomethyl)phenyl]-5-(difluoromethyl)-1,3,4-oxadiazole

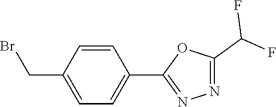 [SMILES: FC(F)c1nnc(-c2ccc(CBr)cc2)o1]

### Step a) N′-(2,2-difluoroacetyl)-4-methyl-benzohydrazide

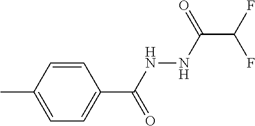 [SMILES: Cc1ccc(C(=O)NNC(=O)C(F)F)cc1]

To a solution of 4-methylbenzohydrazide (3 g, 19.98 mmol, 1 eq) and 2,2-difluoroacetic acid (1.92 g, 1.26 mL, 19.98 mmol, 1 eq) in THF (200 mL) at RT was added DIEA (5.16 g, 6.98 mL, 39.95 mmol, 2 eq) and HATU (8.36 g, 21.97 mmol, 1.1 eq). The mixture was stirred at RT for 3 h. Water and EtOAc were added and the phases separated. The aqueous phase was extracted twice with EtOAc. The combined organic layers were dried over MgSO₄, filtered and the solvent evaporated, to afford a yellow solid (14 g) containing the title compound which was used in the next reaction step without further purification. MS (ESI): 227.1 [M−H]

### Step b) 2-(difluoromethyl)-5-(p-tolyl)-1,3,4-oxadiazole

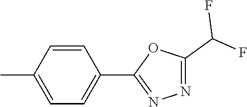

N′-(2,2-difluoroacetyl)-4-methyl-benzohydrazide (14 g, 39.88 mmol, 1.0 eq) was stirred with p-toluenesulfonyl chloride (15.21 g, 79.75 mmol, 2.0 eq) and DIEA (12.88 g, 17.41 mL, 99.69 mmol, 2.5 eq) in DCM (150 mL) for 90 min. Water was added and the phases separated. The aqueous phase was extracted twice with DCM. The combined organic layers were dried over MgSO₄, filtered and evaporated to dryness. The remaining crude material was purified by column chromatography on silica gel (0-30% EtOAC in heptane) to afford 2-(difluoromethyl)-5-(p-tolyl)-1,3,4-oxadiazole (3.1 g, 36%) as light yellow solid. MS (ESI): 211.1 [M+H]⁺

### Step c) 2-[4-(bromomethyl)phenyl]-5-(difluoromethyl)-1,3,4-oxadiazole

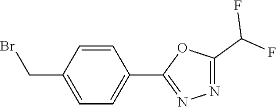 [SMILES: FC(F)c1nnc(-c2ccc(CBr)cc2)o1]

To a solution of 2-(difluoromethyl)-5-(p-tolyl)-1,3,4-oxadiazole (3.1 g, 14.16 mmol, 1.0 eq) in acetonitrile (70 mL) was added N-bromosuccinimide (2.52 g, 14.16 mmol, 1.0 eq) and AIBN (697.53 mg, 4.25 mmol, 0.3 eq). The mixture was stirred at 80° C. overnight. The reaction was poured into brine and extracted 3× with EtOAc. The combined organic phases were dried over anhydrous sodium sulfate, filtered and the solvent evaporated under reduced pressure. The crude was purified by column chromatography on silica gel (0-50% EtOAc in heptane) to afford 2-[4-(bromomethyl)phenyl]-5-(difluoromethyl)-1,3,4-oxadiazole (3.26 g, 70%) as white solid. MS (ESI): 289.0 [M+H]⁺

### Intermediate 19

### 5-[4-(bromomethyl)phenyl]-2-(trifluoromethyl)pyrimidine

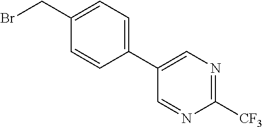 [SMILES: FC(F)(F)c1ncc(-c2ccc(CBr)cc2)cn1]

### Step a) 5-(p-tolyl)-2-(trifluoromethyl)pyrimidine

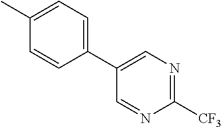 [SMILES: Cc1ccc(-c2cnc(C(F)(F)F)nc2)cc1]

A mixture of p-tolylboronic acid (250 mg, 1.84 mmol, 1.0 eq), 5-bromo-2-(trifluoromethyl)pyrimidine (500.8 mg, 2.21 mmol, 1.2 eq) and K₂CO₃ (762.41 mg, 5.52 mmol, 3.0 eq) in 1,4-dioxane (6.5 mL) and water (650.42 uL) was degassed with argon for 5 min. 1,1-bis(diphenylphosphino)ferrocene dichloro palladium(II) CH₂Cl₂ adduct (26.91 mg, 36.78 μmol, 0.02 eq) was added and the mixture was heated to 80° C. for 3.5 hours. The reaction mixture was partitioned between EtOAc (150 ml) and water (75 ml). The layers were separated and the aqueous layer extracted with EtOAc (2×). The combined organic layers were washed with brine, dried over sodium sulfate, and concentrated under reduced pressure. The remaining crude material was purified by column chromatography on silica geld (0-15% EtOAc in heptanes) to afford 5-(p-tolyl)-2-(trifluoromethyl)pyrimidine (343 mg, 78%) as white solid. MS (ESI): 239.1 [M+H]⁺

### Step b) 5-[4-(bromomethyl)phenyl]-2-(trifluoromethyl)pyrimidine

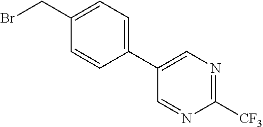 [SMILES: FC(F)(F)c1ncc(-c2ccc(CBr)cc2)cn1]

The title compound was prepared from 5-(p-tolyl)-2-(trifluoromethyl)pyrimidine (343 mg, 15.92 mmol, 1.0 eq) in analogy to general procedure 12 and was obtained as white solid (378 mg, 80%) as white solid. MS (ESI): 317.1 [M+H]⁺

### Intermediate 20

### 3-[4-(bromomethyl)phenyl]-5-(4,4-difluorocyclohexyl)-1,2,4-oxadiazole

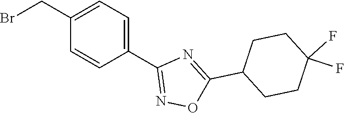 [SMILES: FC1(F)CCC(c2nc(-c3ccc(CBr)cc3)no2)CC1]

### Step a) 5-(4,4-difluorocyclohexyl)-3-(p-tolyl)-1,2,4-oxadiazole

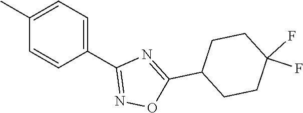 [SMILES: Cc1ccc(-c2noc(C3CCC(F)(F)CC3)n2)cc1]

To a solution of N′-hydroxy-4-methyl-benzamidine (250 mg, 1.66 mmol, 1.0 eq) and ethyl 4,4-difluorocyclohexanecarboxylate (480 mg, 2.5 mmol, 1.5 eq) in DMSO (4 mL) was added NaOH (100 mg, 2.5 mmol, 1.5 eq) at room temperature and the mixture was stirred for 12 h. The reaction mixture was poured into water, a yellow precipitate formed and the mixture was filtered. The filter cake was dissolved in EtOAc, dried over sodium sulfate, filtered, and concentrated under reduced pressure to give 5-(4,4-difluorocyclohexyl)-3-(p-tolyl)-1,2,4-oxadiazole (197 mg, 0.71 mmol, 37% yield) as light yellow solid. MS (ESI): 279.1 [M+H]⁺

### Step b) 3-[4-(bromomethyl)phenyl]-5-(4,4-difluorocyclohexyl)-1,2,4-oxadiazole

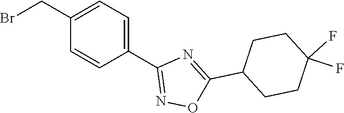 [SMILES: FC1(F)CCC(c2nc(-c3ccc(CBr)cc3)no2)CC1]

The tile compound was prepared from 5-(4,4-difluorocyclohexyl)-3-(p-tolyl)-1,2,4-oxadiazole (1130 mg, 4.06 mmol) in analogy to general procedure 12 and was obtained as light yellow solid (670 mg, 1.88 mmol, 38% yield). MS (ESI): 359.0 [M+H]⁺

### Intermediate 22

### 1-[4-(bromomethyl)phenyl]-3-(trifluoromethyl)-1,2,4-triazole

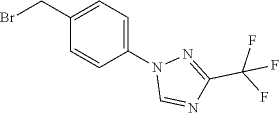 [SMILES: FC(F)(F)c1ncn(-c2ccc(CBr)cc2)n1]

### Step a) 1-(p-tolyl)-3-(trifluoromethyl)-1,2,4-triazole

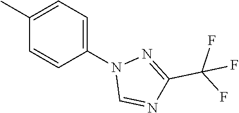 [SMILES: Cc1ccc(-n2cnc(C(F)(F)F)n2)cc1]

p-Tolylboronic acid (500 mg, 3.68 mmol, 1.0 eq) and 3-(trifluoromethyl)-1H-1,2,4-triazole (504 mg, 3.68 mmol, 1.0 eq) was dissolved in DMF (2 mL). Pyridine (581.8 mg, 595 μL, 7.36 mmol, 2.0 eq) and copper (powder) (46.7 mg, 735 μmol, 0.2 eq) was added. The reaction mixture was stirred at 50° C. overnight. 1N HCl was added and the mixture extracted EtOAC (3×), dried over MgSO₄, filtered, and concentrated under reduced pressure. The remaining crude material was purified by column chromatography on silica gel (0-50% EtOAc in heptanes) to give 1-(p-tolyl)-3-(trifluoromethyl)-1,2,4-triazole (316 mg, 38%) as white crystalline. MS (ESI): 228.2 [M+H]⁺

### Step b) 1-[4-(bromomethyl)phenyl]-3-(trifluoromethyl)-1,2,4-triazole

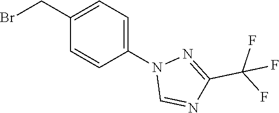 [SMILES: FC(F)(F)c1ncn(-c2ccc(CBr)cc2)n1]

The title compound was prepared from 1-(p-tolyl)-3-(trifluoromethyl)-1,2,4-triazole (315 mg, 1.39 mmol) in analogy to general procedure 12 and was obtained as white solid (351 mg, 68%). MS (ESI): 308.0 [M+H]⁺

### Intermediate 23

### 2-[4-(bromomethyl)phenyl]-5-(trifluoromethyl)oxazole

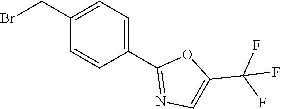 [SMILES: FC(F)(F)c1cnc(-c2ccc(CBr)cc2)o1]

### Step a) 4-methyl-N-(3,3,3-trifluoro-2-oxo-propyl)benzamide

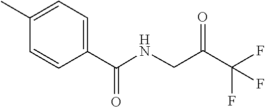 [SMILES: Cc1ccc(C(=O)NCC(=O)C(F)(F)F)cc1]

The solution of 4-methylbenzamide (500 mg, 3.7 mmol, 1.0 eq) in toluene (10 ml) was added 3-chloro-1,1,1-trifluoroacetone (1.08 g, 7.4 mmol, 2.0 eq) and the mixture was stirred at 110° C. for 24 h. The mixture was poured into water and extracted with EtOAc twice. The combined organic phase was washed with brine dried over sodium sulfate and concentrated to dryness. The remaining crude was purified by column chromatography on silica gel (0-95% EtOAc in petroleum ether) to afford 4-methyl-N-(3,3,3-trifluoro-2-oxo-propyl)benzamide (1.2 g mg, 99% yield) as light yellow solid. MS (ESI): 246.1 [M+H]⁺

### Step b) 2-(p-tolyl)-5-(trifluoromethyl)oxazole

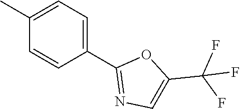 [SMILES: Cc1ccc(-c2ncc(C(F)(F)F)o2)cc1]

The solution of 4-methyl-N-(3,3,3-trifluoro-2-oxo-propyl)benzamide (1.2 g, 4.89 mmol, 1.0 eq) in pyridine (150 mL) was added POCl₃ (30.0 mL, 321.85 mmol) and the mixture was stirred at room temperature for 3 h. The reaction mixture poured into a cold (0° C.) solution of saturated aqueous NaHCO₃ (100 mL), and extracted with EtOAc (100 mL) twice. The combined organic layers were then washed with water (25 mL) and brine (25 mL), dried over sodium sulfate, and concentrated in vacuo. The remaining residue was purified by column chromatography on slica gel to afford the title product (400 mg, 36% yield) as white solid. MS (ESI): 228.1 [M+H]⁺

### Step c) 2-[4-(bromomethyl)phenyl]-5-(trifluoromethyl)oxazole

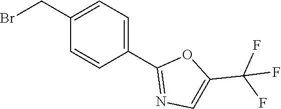 [SMILES: FC(F)(F)c1cnc(-c2ccc(CBr)cc2)o1]

The title compound was prepared from 2-(p-tolyl)-5-(trifluoromethyl)oxazole (566.0 mg, 2.49 mmol, 1.0 eq) in analogy to general procedure 12 and was obtained as colorless solid (450 mg, 45%). MS (ESI): 307.9 [M+H]⁺

### Intermediate 24

### 5-[4-(bromomethyl)phenyl]-1-methyl-3-(trifluoromethyl)pyrazole

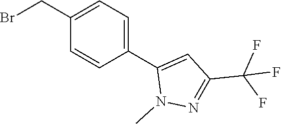 [SMILES: Cn1nc(C(F)(F)F)cc1-c1ccc(CBr)cc1]

### Step a) [4-[2-methyl-5-(trifluoromethyl)pyrazol-3-yl]phenyl]methanol

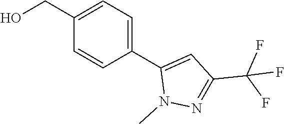 [SMILES: Cn1nc(C(F)(F)F)cc1-c1ccc(CO)cc1]

[2-methyl-5-(trifluoromethyl)pyrazol-3-yl]boronic acid (364.59 mg, 1.88 mmol, 1.1 eq) and 4-iodobenzyl alcohol (400 mg, 1.71 mmol, 1.0 eq) were dissolved in 1,4-dioxane (4.68 mL). To this mixture, a solution of Na₂CO₃ (453 mg, 4.27 mmol, 2.5 eq) in water (2.12 mL) was added and the mixture degassed by bubbling Argon through the reaction for 10 min. Pd(PPh₃)₄ (197.5 mg, 170.9 mol, 0.1 eq) was added and the mixture stirred at 100° C. overnight. The mixture was cooled down to room temperature and poured diluted with water and EtOAc. The aqueous layer was extracted with EtOAc twice, combined and dried with sodium sulfate. The mixture was filtered and concentrated under reduced pressure. The remaining crude was purified by column chromatography on silica gel (0-50% EtOAc in heptanes) to afford [4-[2-methyl-5-(trifluoromethyl)pyrazol-3-yl]phenyl]methanol (210 mg, 48%) as colorless oil. MS (ESI): 257.1 [M+H]⁺

### Step b) 5-[4-(bromomethyl)phenyl]-1-methyl-3-(trifluoromethyl)pyrazole

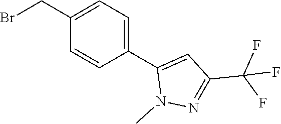 [SMILES: Cn1nc(C(F)(F)F)cc1-c1ccc(CBr)cc1]

To a solution of [4-[2-methyl-5-(trifluoromethyl)pyrazol-3-yl]phenyl]methanol (210 mg, 819.6 μmol, 1.0 eq) in DCM (5.46 mL) at 0° C. was added PBr₃ (110.9 mg, 38.65 uL, 409 μmol, 0.5 eq) dropwise. The resulting reaction was stirred at at 0° C. for 90 min. After 2 hr, PBr₃ (110.9 mg, 38.65 uL, 410 mol, 0.5 eq) was added and stirred for 16 h at room temperature. The reaction mixture was diluted with water and sat. aq. NaHCO₃. The organic layer was extracted with DCM three times, dried over sodium sulfate, filtered and concentrated under reduced pressure. The remaining crude was purified using column chromatography on silica gel (0-60% EtOAc in heptanes) to afford 5-[4-(bromomethyl)phenyl]-1-methyl-3-(trifluoromethyl)pyrazole (156.7 mg, 60%) as white solid. MS (ESI): 319.0 [M+H]⁺

### Intermediate 25

### 2-[4-(bromomethyl)phenyl]-5-(difluoromethoxy)pyridine

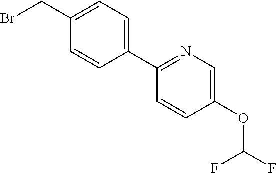 [SMILES: FC(F)Oc1ccc(-c2ccc(CBr)cc2)nc1]

### Step a) 5-(difluoromethoxy)-2-(p-tolyl)pyridine

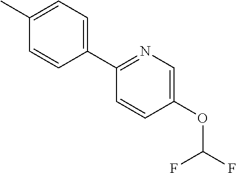 [SMILES: Cc1ccc(-c2ccc(OC(F)F)cn2)cc1]

A mixture of p-tolylboronic acid (500 mg, 3.68 mmol, 1.0 eq), 2-bromo-5-(difluoromethoxy)pyridine (988 mg, 4.41 mmol, 1.2 eq) and K₂CO₃ (1.52 g, 11.03 mmol, 3.0 eq) in 1,4-dioxane (13 mL) and water (1.3 mL) was degassed with argon for 5 min. 1,1-bis(diphenylphosphino)ferrocene dichloro palladium(II) CH₂Cl₂ adduct (53.82 mg, 73.55 μmol, 0.02 eq) was added at room temperature. The reaction mixture was heated to 80° C. and stirred for 1 hour. The reaction mixture was partitioned between EtOAc and water. The layers were separated and the aqueous layer was extracted with EtOAc (2×). The combined organic layers were washed with brine, dried over MgSO₄, filtered and concentrated under reduced pressure. The remaining crude material was purified by column chromatography (0-20% EtOAc in heptanes) to afford 5-(difluoromethoxy)-2-(p-tolyl)pyridine (613 mg, 71%) as colorless liquid. MS (ESI): 236.1 [M+H]⁺

### Step b) 2-[4-(bromomethyl)phenyl]-5-(difluoromethoxy)pyridine

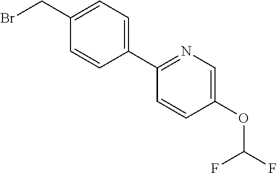 [SMILES: FC(F)Oc1ccc(-c2ccc(CBr)cc2)nc1]

The title compound was prepared from 5-(difluoromethoxy)-2-(p-tolyl)pyridine (613 mg, 2.61 mmol) in analogy to general procedure 12 and was obtained as light yellow liquid (456 mg, 56%). MS (ESI): 314.1 [M+H]⁺

### Intermediate 26

### 2-[4-(bromomethyl)phenyl]-5-(1,1-difluoroethyl)pyridine

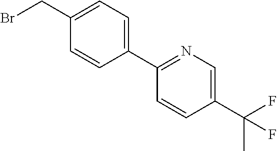 [SMILES: CC(F)(F)c1ccc(-c2ccc(CBr)cc2)nc1]

### Step a) 5-(1,1-difluoroethyl)-2-(p-tolyl)pyridine

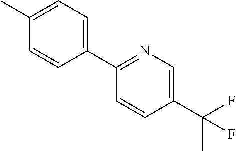 [SMILES: Cc1ccc(-c2ccc(C(C)(F)F)cn2)cc1]

A mixture of p-tolylboronic acid (500 mg, 3.68 mmol, 1.0 eq), 2-chloro-5-(1,1-difluoroethyl)pyridine (783.67 mg, 4.41 mmol, 1.2 eq) and K₂CO₃ (1.52 g, 11.03 mmol, 3.0 eq) in 1,4-dioxane (13 mL) and water (1.3 mL) was degassed with argon for 5 min. 1,1-bis(diphenylphosphino)ferrocene dichloro palladium(II) CH₂Cl₂ adduct (53.82 mg, 73.55 μmol, 0.02 eq) was added at room temperature. The reaction mixture was heated to 80° C. and stirred for 1.5 hours. The reaction mixture was partitioned between EtOAc and water. The layers were separated and the aqueous layer was extracted with EtOAc (2×). The combined organic layers were washed with brine, dried over MgSO₄, filtered and concentrated under reduced pressure. The remaining crude material was purified by column chromatography (0-20% EtOAc in heptanes) to afford 5-(1,1-difluoroethyl)-2-(p-tolyl)pyridine (740 mg, 79%) as white crystalline solid. MS (ESI): 234.2 [M+H]⁺

### Step b) 2-[4-(bromomethyl)phenyl]-5-(1,1-difluoroethyl)pyridine

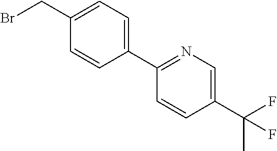 [SMILES: CC(F)(F)c1ccc(-c2ccc(CBr)cc2)nc1]

The title compound was prepared from 5-(1,1-difluoroethyl)-2-(p-tolyl)pyridine (740 mg, 3.17 mmol) in analogy to general procedure 12 and was obtained as white crystalline solid (609 mg, 49%). MS (ESI): 314.1 [M+H]⁺

### Intermediate 27

### 2-[4-(bromomethyl)phenyl]-4-(difluoromethoxy)pyridine

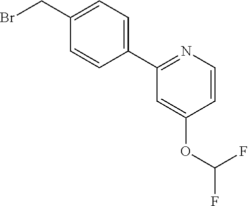 [SMILES: FC(F)Oc1ccnc(-c2ccc(CBr)cc2)c1]

### Step a) 4-(difluoromethoxy)-2-(p-tolyl)pyridine

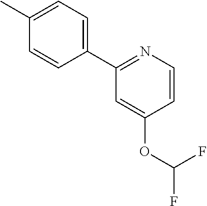 [SMILES: Cc1ccc(-c2cc(OC(F)F)ccn2)cc1]

A mixture of p-tolylboronic acid (250 mg, 1.84 mmol, 1.0 eq), 2-bromo-4-(difluoromethoxy)pyridine (494.26 mg, 2.21 mmol, 1.2 eq) and K₃PO₄ (741.6 mg, 3.49 mmol, 1.9 eq) in 1,4-dioxane (9.19 mL) and water (1.31 mL) was degased with argon for 10 min in the ultrasonic bath. Then, 1,1-bis(diphenylphosphino)ferrocene dichloro palladium(II) CH₂Cl₂ adduct (152 mg, 183.9 μmol, 0.1 eq) was added and the reaction heated to 80° C. for 3 hours. The reaction mixture was partitioned between EtOAc and water. The layers were separated and the aqueous layer extracted with EtAOc (2×). The combined organic layers were washed with brine, dried over sodium sulfate and concentrated under reduced pressure. The remaining residue was purified by column chromatography on silica gel (0-20% EtOAc in heptanes) to yield the title compound as colorless liquid (461.5 mg, 96%). MS (ESI): 236.0 [M+H]⁺

### Step b) 2-[4-(bromomethyl)phenyl]-4-(difluoromethoxy)pyridine

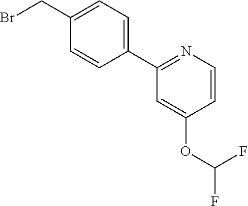 [SMILES: FC(F)Oc1ccnc(-c2ccc(CBr)cc2)c1]

The title compound was prepared from 4-(difluoromethoxy)-2-(p-tolyl)pyridine (440 mg, 1.68 mmol) in analogy to general procedure 12 and was obtained as white solid (171 mg, 31%). MS (ESI): 315.9 [M+H]⁺

### Intermediate 28

### 4-[4-(bromomethyl)phenyl]-2-(trifluoromethyl)pyridine

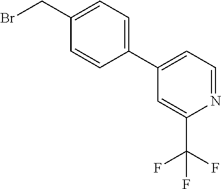 [SMILES: FC(F)(F)c1cc(-c2ccc(CBr)cc2)ccn1]

### Step a) 2-(trifluoromethyl)-4-(p-tolyl)pyridine

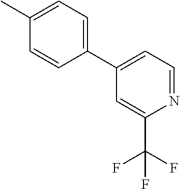 [SMILES: Cc1ccc(-c2ccnc(C(F)(F)F)c2)cc1]

A mixture of p-tolylboronic acid (250 mg, 1.84 mmol, 1.0 eq), 2-bromo-4-(trifluoromethyl)pyridine (514 mg, 2.21 mmol, 1.2 eq) and K₂CO₃ (762.41 mg, 5.52 mmol, 3.0 eq) in 1,4-dioxane (6.5 mL) and water (650.42 uL) was degassed with argon for 5 min. 1,1-bis(diphenylphosphino)ferrocene dichloro palladium(II) CH₂Cl₂ adduct (26.91 mg, 36.78 μmol, 0.02 eq) was added at room temperature. The reaction mixture was heated to 80° C. and stirred for 3.5 hours. The reaction mixture was partitioned between EtOAc and water. The layers were separated and the aqueous layer was extracted with EtOAc (2×). The combined organic layers were washed with brine, dried over MgSO₄, filtered and concentrated under reduced pressure. The remaining crude material was purified by column chromatography (0-20% EtOAc in heptanes) to afford 2-(trifluoromethyl)-4-(p-tolyl)pyridine (403 mg, 90%) as white solid. MS (ESI): 238.1 [M+H]⁺

### Step b) 4-[4-(bromomethyl)phenyl]-2-(trifluoromethyl)pyridine

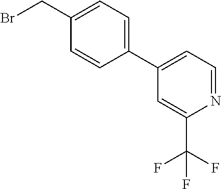 [SMILES: FC(F)(F)c1cc(-c2ccc(CBr)cc2)ccn1]

The title compound was prepared from 2-(trifluoromethyl)-4-(p-tolyl)pyridine (403 mg, 1.65 mmol) in analogy to general procedure 12 and was obtained as white solid (475 mg, 81%). MS (ESI): 316.0 [M+H]⁺

### Intermediate 29

### 2-[4-(bromomethyl)phenyl]-5-(trifluoromethoxy)pyrazine

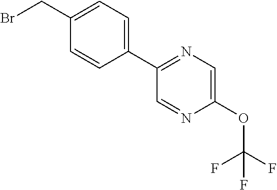 [SMILES: FC(F)(F)Oc1cnc(-c2ccc(CBr)cc2)cn1]

### Step a) 2-(p-tolyl)-5-(trifluoromethoxy)pyrazine

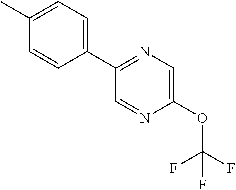 [SMILES: Cc1ccc(-c2cnc(OC(F)(F)F)cn2)cc1]

2-chloro-5-(trifluoromethoxy)pyrazine (50 mg, 252 μmol, 1.0 eq) was dissolved in 1,4-dioxane (1.26 mL) and water (1.26 mL). K₂CO₃ (87 mg, 630 μmol, 2.5 eq), p-tolylboronic acid (34.24 mg, 251.85 mol, 1.0 eq) and 1,1-bis(diphenylphosphino)ferrocene dichloro palladium(II) CH₂Cl₂ adduct (10.28 mg, 12.6 μmol, 0.05 eq) were added and the reaction mixture was heated up to 80° C. and stirred for 2 h. The reaction was poured into water and extracted twice with EtOAc. The organic layers were combined, washed with brine, dried over sodium sulfate and concentrated under reduced pressure. The remaining crude material was purified by column chromatography on silica gel (0-10% EtOAc in heptanes) to afford the title compound as white solid (49.8 mg, 78%). MS (ESI): 255.0 [M+H]⁺

### Step b) 2-[4-(bromomethyl)phenyl]-5-(trifluoromethoxy)pyrazine

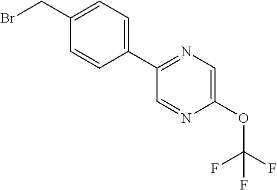 [SMILES: FC(F)(F)Oc1cnc(-c2ccc(CBr)cc2)cn1]

The title compound was prepared from 2-(p-tolyl)-5-(trifluoromethoxy)pyrazine (49.8 mg, 0.2 mmol) in analogy to general procedure 12 and was obtained as white solid (55.4 mg, 78%). MS (ESI): 332.9 [M+H]⁺

### Intermediate 30

### 6-[4-(bromomethyl)phenyl]-2-methyl-3-(trifluoromethyl)pyridine

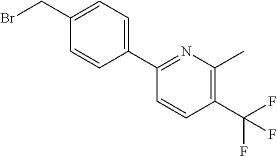 [SMILES: Cc1nc(-c2ccc(CBr)cc2)ccc1C(F)(F)F]

### Step a) 2-methyl-6-(p-tolyl)-3-(trifluoromethyl)pyridine

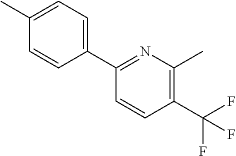 [SMILES: Cc1ccc(-c2ccc(C(F)(F)F)c(C)n2)cc1]

6-chloro-2-methyl-3-(trifluoromethyl)pyridine (431.53 mg, 2.21 mmol, 1.0 eq) was stirred with p-tolylboronic acid (300 mg, 2.21 mmol, 1.0 eq), 1,1-bis(diphenylphosphino)ferrocene dichloro palladium(II) CH₂Cl₂ adduct (90 mg, 110.3 μmol, 0.05 eq) and K₂CO₃ (762.4 mg, 5.52 mmol, 2.5 eq) in 1,4-dioxane (7.2 mL) and water (720 uL) at 80° C. for 3 h. The reaction was concentrated under reduced pressure and directly purified by column chromatography on silica (0-30% EtOAc in heptanes) to afford 2-methyl-6-(p-tolyl)-3-(trifluoromethyl)pyridine (305 mg, 53%) as light yellow solid. MS (ESI): 252.1 [M+H]⁺

### Step b) 6-[4-(bromomethyl)phenyl]-2-methyl-3-(trifluoromethyl)pyridine

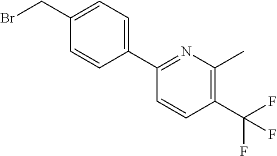 [SMILES: Cc1nc(-c2ccc(CBr)cc2)ccc1C(F)(F)F]

The title compound was prepared from 2-methyl-6-(p-tolyl)-3-(trifluoromethyl)pyridine (305 mg, 1.21 mmol) in analogy to general procedure 12 and was obtained as white solid (344 mg, 76%). MS (ESI): 332.0 [M+H]⁺

### Intermediate 31

### 4-[4-(bromomethyl)phenyl]-1-(trifluoromethyl)pyrazole

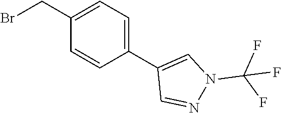 [SMILES: FC(F)(F)n1cc(-c2ccc(CBr)cc2)cn1]

### Step a) 4-(p-tolyl)-1-(trifluoromethyl)pyrazole

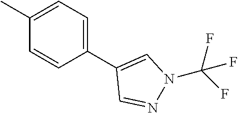 [SMILES: Cc1ccc(-c2cnn(C(F)(F)F)c2)cc1]

4-bromo-1-(trifluoromethyl)pyrazole (63.25 mg, 294 μmol, 1.0 eq) was dissolved in 1,4-dioxane (1.47 mL) and water (1.47 mL). K₂CO₃ (101.65 mg, 735.51 μmol, 2.5 eq), p-tolylboronic acid (40 mg, 294.2 mol, 1.0 eq) and 1,1-bis(diphenylphosphino)ferrocene dichloro palladium(II) CH₂Cl₂ adduct (12.01 mg, 14.71 μmol, 0.05 eq) were added to the solution and the reaction mixture was heated to 100° C. for 1 h. The reaction mixture was cooled to room temperature, diluted with water and washed 3× with EtOAc. The organic layers were combined, dried with sodium sulfate, filtered and concentrated under reduced pressure. The remaining cruded was purified by column chromatography on silica geld (0-20% EtOAc in heptanes). to give 4-(p-tolyl)-1-(trifluoromethyl)pyrazole (41 mg, 59%) as light yellow oil. MS (ESI): 227.0 [M+H]⁺

### Step b) 4-[4-(bromomethyl)phenyl]-1-(trifluoromethyl)pyrazole

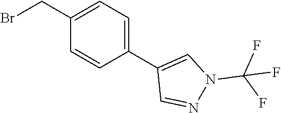 [SMILES: FC(F)(F)n1cc(-c2ccc(CBr)cc2)cn1]

The title compound as prepared from 4-(p-tolyl)-1-(trifluoromethyl)pyrazole (29.9 mg, 132 mol) in analogy to general procedure 12 and was obtained as white solid (14.4 mg, 31%) formation. MS (ESI): 306.9 [M+H]⁺

### Intermediate 32

### 2-[4-(bromomethyl)phenyl]-4-(trifluoromethyl)pyridine

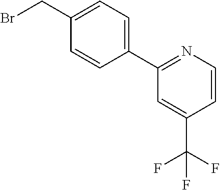 [SMILES: FC(F)(F)c1ccnc(-c2ccc(CBr)cc2)c1]

### Step a) 2-(p-tolyl)-4-(trifluoromethyl)pyridine

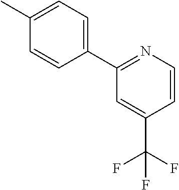 [SMILES: Cc1ccc(-c2cc(C(F)(F)F)ccn2)cc1]

A mixture of p-tolylboronic acid (250 mg, 1.84 mmol, 1.0 eq), 2-bromo-4-(trifluoromethyl)pyridine (514 mg, 2.21 mmol, 1.2 eq) and K₂CO₃ (762.41 mg, 5.52 mmol, 3.0 eq) in 1,4-dioxane (6.5 mL) and water (650 uL) was degassed with argon for 5 min. 1,1-bis(diphenylphosphino)ferrocene dichloro palladium(II) CH₂Cl₂ adduct (26.91 mg, 36.78 μmol, 0.02 eq) was added at room temperature. The reaction mixture was heated to 80° C. and stirred for 3.5 hours. The reaction mixture was partitioned between EtOAc and water. The layers were separated and the aqueous layer was extracted with EtOAc (2×). The combined organic layers were washed with brine, dried over MgSO₄, filtered and concentrated under reduced pressure. The remaining crude material was purified by column chromatography (0-15% EtOAc in heptanes) to afford 2-(p-tolyl)-4-(trifluoromethyl)pyridine (374 mg, 80%) as colorless oil. MS (ESI): 238.2 [M+H]⁺

### Step b) 2-[4-(bromomethyl)phenyl]-4-(trifluoromethyl)pyridine

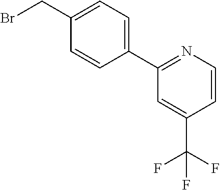 [SMILES: FC(F)(F)c1ccnc(-c2ccc(CBr)cc2)c1]

The title compound was prepared from 2-(p-tolyl)-4-(trifluoromethyl)pyridine (3.49 g, 15.92 mmol) in analogy to general procedure 12 and was obtained as white solid (371 mg, 68%). MS (ESI): 316.0 [M+H]⁺

### Intermediate 33

### 2-[4-(bromomethyl)phenyl]-5-(trifluoromethoxy)pyrimidine

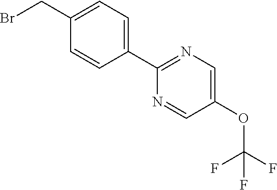 [SMILES: FC(F)(F)Oc1cnc(-c2ccc(CBr)cc2)nc1]

### Step a) 2-(p-tolyl)-5-(trifluoromethoxy)pyrimidine

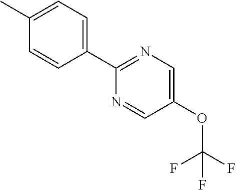 [SMILES: Cc1ccc(-c2ncc(OC(F)(F)F)cn2)cc1]

2-chloro-5-(trifluoromethoxy)pyrimidine (50 mg, 251.85 μmol, 1.0 eq) was dissolved in 1,4-dioxane (1.26 mL) and water (1.26 mL). p-tolylboronic acid (34.24 mg, 251.85 μmol, 1.0 eq), K₂CO₃ (87.02 mg, 629.63 μmol, 2.5 eq) and 1,1-bis(diphenylphosphino)ferrocene dichloro palladium(II) CH₂Cl₂ adduct (10.28 mg, 12.59 μmol, 0.05 eq) were added and the reaction mixture was heated to 80° C. and stirred for 2 h. The reaction was poured into water and extracted twice with EtOAc. The organic phases were combined, washed with brine, dried over sodium sulfate and concentrated under reduced pressure. The crude material was purified by column chromatography on silica gel (0-10% EtOAc in heptanes) to afford 2-(p-tolyl)-5-(trifluoromethoxy)pyrimidine (52.8 mg, 83%) as white solid. MS (ESI): 255.0 [M+H]⁺

### Step b) 2-[4-(bromomethyl)phenyl]-5-(trifluoromethoxy)pyrimidine

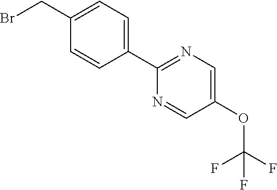 [SMILES: FC(F)(F)Oc1cnc(-c2ccc(CBr)cc2)nc1]

The title compound was prepared from 2-(p-tolyl)-5-(trifluoromethoxy)pyrimidine (52.8 mg, 207.7 mol) in analogy to general procedure 12 and was obtained as white solid (56.4 mg, 73%). MS (ESI): 333.0 [M+H]⁺

### Intermediate 34

### 2-[4-(bromomethyl)phenyl]-5-cyclopropyl-pyridine

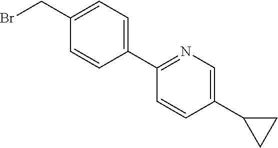 [SMILES: BrCc1ccc(-c2ccc(C3CC3)cn2)cc1]

### Step a) [4-(5-cyclopropyl-2-pyridyl)phenyl]methanol

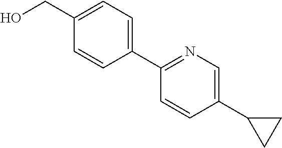 [SMILES: OCc1ccc(-c2ccc(C3CC3)cn2)cc1]

2-bromo-5-cyclopropyl-pyridine (46.9 mg, 236.8 μmol, 1.0 eq) was dissolved in 1,4-dioxane (1.18 mL) and water (1.18 mL). [4-(4,4,5,5-tetramethyl-1,3,2-dioxaborolan-2-yl)phenyl]methanol (55.43 mg, 236.8 μmol, 1.0 eq), K₂CO₃ (81.82 mg, 591.99 μmol, 2.5 eq) and 1,1-bis(diphenylphosphino)ferrocene dichloro palladium(II) CH₂Cl₂ adduct (9.67 mg, 11.84 μmol, 0.05 eq) were added to the solution. The reaction mixture was heated up to 80° C. and stirred for 45 minutes. The reaction was poured into water and extracted twice with EtOAc. The organic phases were combined, washed with brine, dried over sodium sulfate and concentrated under reduced pressure. The remaining crude material was purified by column chromatography on silica geld (0-100% EtOAc in heptanes) to afford [4-(5-cyclopropyl-2-pyridyl)phenyl]methanol (33.2 mg, 62%) as white solid. MS (ESI): 226.0 [M+H]⁺

### Step b) 2-[4-(bromomethyl)phenyl]-5-cyclopropyl-pyridine

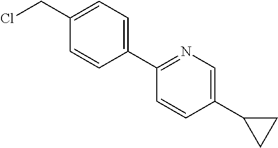 [SMILES: ClCc1ccc(-c2ccc(C3CC3)cn2)cc1]

[4-(5-cyclopropyl-2-pyridyl)phenyl]methanol (33.2 mg, 147.37 μmol, 1.0 eq) and Et₃N (29.82 mg, 41.08 uL, 294.73 μmol, 2.0 eq) were dissolved in DCM (1000 uL). The solution was cooled to 0° C. and p-TsCl (28.1 mg, 147.37 μmol, 1.0 eq) was added to the solution. The reaction was allowed to warm up to RT and stirred for 3 h. p-TsCl (84.3 mg, 442 μmol, 3.0 eq) was added to the reaction mixture and stirred for at room temperature overnight. The reaction was poured into ice water and extracted twice with EtOAc. The organic phases were combined, washed with brine, dried over sodium sulfate and concentrated under reduced pressure. The remaining crude material was purified by column chromatography on silica gel (0-100% EtOAc in heptanes) to afford 2-[4-(chloromethyl)phenyl]-5-cyclopropyl-pyridine (15.6 mg, 43%) as white solid. MS (ESI): 244.0 [M+H]⁺

### Intermediate 35

### 2-[4-(bromomethyl)phenyl]-5-(trifluoromethyl)tetrazole

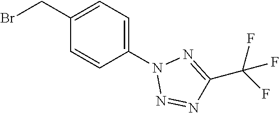 [SMILES: FC(F)(F)c1nnn(-c2ccc(CBr)cc2)n1]

### Step a) 2-(p-tolyl)-5-(trifluoromethyl)tetrazole

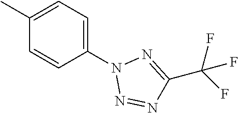 [SMILES: Cc1ccc(-n2nnc(C(F)(F)F)n2)cc1]

4-methylbenzenediazonium;tetrafluoroborate (100 mg, 485.55 μmol, 1.0 eq), 4-methyl-N-[(E)-2,2,2-trifluoroethylideneamino]benzenesulfonamide (161.59 mg, 606.94 μmol, 1.25 eq) and sodium tert-butoxide (93.32 mg, 971.11 μmol, 2.0 eq) were dissolved in DMSO (1.94 mL) and the reaction was stirred at 80° C. for 2 hours. The reaction was quenched by addition of water and extracted with EtOAc (2×). The combined organic layer was washed water and brine. The organic phase was dried over sodium sulfate, filtered and concentrated under reduced pressure. The remaining crude was purified by column chromatography on silica gel (0-30% EtOAc in heptanes) to afford 2-(n-tolyl)-5-(trifluoromethyl)tetrazole (52 mg, 45%) as light yellow solid. ¹H-NMR (CDCl3, 400 MHz): 8.04 (d, J=8.4 Hz, 2H), 7.40 (d, J=8.4 Hz, 2H), 2.48 (s, 3H).

### Step b) 2-[4-(bromomethyl)phenyl]-5-(trifluoromethyl)tetrazole

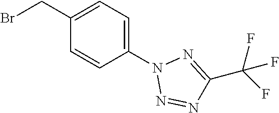 [SMILES: FC(F)(F)c1nnn(-c2ccc(CBr)cc2)n1]

The title compound was prepared from 2-(p-tolyl)-5-(trifluoromethyl)tetrazole (300 mg, 1.31 mmol) in analogy to general procedure 12 and was obtained as a yellow oil (200 mg, 0.65 mmol, 50% yield). ¹H-NMR (CDCl3, 400 MHz): 8.17 (d, J=8.8 Hz, 2H), 7.64 (d, J=8.8 Hz, 2H), 4.56 (s, 2H).

### Intermediate 37

### 2-[4-(chloromethyl)phenyl]-4-(trifluoromethyl)oxazole

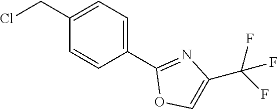 [SMILES: FC(F)(F)c1coc(-c2ccc(CCl)cc2)n1]

### Step a) [4-[4-(trifluoromethyl)oxazol-2-yl]phenyl]methanol

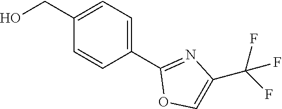 [SMILES: OCc1ccc(-c2nc(C(F)(F)F)co2)cc1]

The solution of 2-bromo-4-(trifluoromethyl)oxazole (1.3 g, 6.02 mmol, 1.0 eq), 1,1-bis(diphenylphosphino)ferrocene dichloro palladium(II) CH₂Cl₂ adduct (333.7 mg, 0.6 mmol, 0.1 eq), 4-(hydroxymethyl)phenylboronic acid (457.4 mg, 3.0 mmol, 0.5 eq), palladium (II) acetate (67.6 mg, 0.3 mmol, 0.05 eq), sodium carbonate (1914 mg, 18.06 mmol, 3.0 eq) in NMP (10 mL) and water (2 mL) was stirred at 90° C. for 8 h under inert atmosphere. The solution was poured into water and extracted with EtOAc (3×). The combined organic phase was washed with brine, dried over sodium sulfate, filtered and concentrated under reduced pressure. The remaining residue was purified by column chromatography on slica gel (5-30% EtOAc in petroleum ether) to give a light brown oil containing [4-[4-(trifluoromethyl)oxazol-2-yl]phenyl]methanol (2.2 g), which was used in the next step without further purification. MS (ESI): 244.1 [M+H]⁺

### Step b) 2-[4-(chloromethyl)phenyl]-4-(trifluoromethyl)oxazole

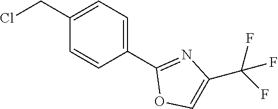 [SMILES: FC(F)(F)c1coc(-c2ccc(CCl)cc2)n1]

A solution of [4-[4-(trifluoromethyl)oxazol-2-yl]phenyl]methanol (2.0 g, 8.22 mmol, 1.0 eq) and thionyl chloride (0.6 mL, 8.22 mmol, 1.0 eq) in DCM (20 mL) was stirred at room temperature for 1 h. The reaction was quenched upon addition of sat. aq. NaHCO₃. The phases were separated and the aqueous phase washed twice with DCM. The combined organic phase was washed with brine, dried over sodium sulfate, filtered and concentrated under reduced pressure to afford the title product as light yellow oil (1.1 g, 4.2 mmol, 38% yield). MS (ESI): 262.0 [M+H]⁺

### Intermediate 38

### 2-[4-(bromomethyl)phenyl]-5-(trifluoromethoxy)pyridine

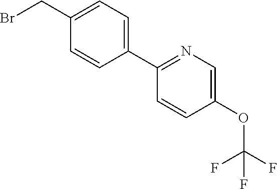 [SMILES: FC(F)(F)Oc1ccc(-c2ccc(CBr)cc2)nc1]

### Step a) 2-(p-tolyl)-5-(trifluoromethoxy)pyridine

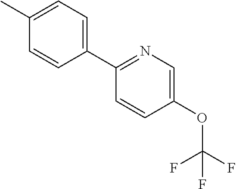 [SMILES: Cc1ccc(-c2ccc(OC(F)(F)F)cn2)cc1]

Palladium (II) acetate (22.73 mg, 101.25 μmol, 0.02 eq) was dissolved in THF (15.3 mL), which was previously degassed with Argon, and heated to 45° C. XantPhos (115.1 mg, 203 μmol, 0.04 eq) was added and the mixture was allowed to stir for 20 minutes. 2-chloro-5-(trifluoromethoxy)pyridine (1000 mg, 683.53 uL, 5.06 mmol, 1.0 eq), p-tolylboronic acid (825.9 mg, 6.07 mmol, 1.2 eq) and K₂CO₃ (1.4 g, 10.12 mmol, 2.0 eq) were added all at once along with water (3 mL) and the reaction mixture was stirred at 45° C. for 4 hours. The reaction mixture was diluted with water and concentrated under reduced pressure to remove most of the THF. The reaction mixture was further diluted with water and then extracted using EtOAc (3×). The combined organic layers were washed with water and brine, dried over sodium sulfate, filtered and concentrated under reduced pressure. The remaining residue was purified by column chromatography on silica gel (0-30% EtOAc in heptanes) to afford the title compound as white solid (1016 mg, 79%). MS (ESI): 254.1 [M+H]⁺

### Step b) 2-[4-(bromomethyl)phenyl]-5-(trifluoromethoxy)pyridine

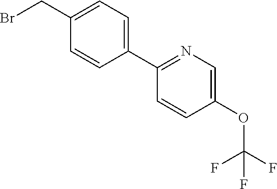 [SMILES: FC(F)(F)Oc1ccc(-c2ccc(CBr)cc2)nc1]

The title compound was prepared from 2-(p-tolyl)-5-(trifluoromethoxy)pyridine (1016 mg, 4.01 mmol) in analogy to general procedure 12 and was obtained as white solid (972 mg, 63%). MS (ESI): 332.0 [M+H]⁺

### Intermediate 39

### 2-[4-(bromomethyl)phenyl]-4-(trifluoromethoxy)pyridine

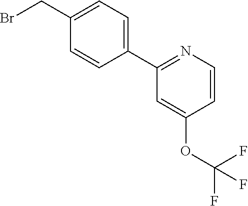 [SMILES: FC(F)(F)Oc1ccnc(-c2ccc(CBr)cc2)c1]

### Step a) 2-(p-tolyl)-4-(trifluoromethoxy)pyridine

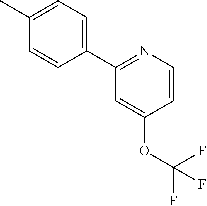 [SMILES: Cc1ccc(-c2cc(OC(F)(F)F)ccn2)cc1]

A mixture of p-tolylboronic acid (20 mg, 147.1 μmol, 1.0 eq), 2-bromo-4-(difluoromethoxy)pyridine (39.54 mg, 176.52 μmol, 1.2 eq) and K₃PO₄ (59.33 mg, 279.49 μmol, 1.9 eq) in 1,4-dioxane (736 uL) and water (105 uL) was degassed with argon for 10 min in the ultrasonic bath. Then, 1,1-bis(diphenylphosphino)ferrocene dichloro palladium(II) CH₂Cl₂ adduct (12.16 mg, 14.71 μmol, 0.1 eq) was added and the reaction heated to 80° C. for 3 hours. The reaction mixture was partitioned between EtOAc and water. The layers were separated and the aqueous layer extracted with EtAOc (2×). The combined organic layers were washed with brine, dried over sodium sulfate and concentrated under reduced pressure. The remaining residue was purified by column chromatography on silica gel (0-20% EtOAc in heptanes) to afford the title compound as colorless liquid (376 mg, 79%). MS (ESI): 254.0 [M+H]⁺

### Step b) 2-[4-(bromomethyl)phenyl]-4-(trifluoromethoxy)pyridine

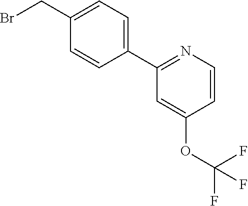 [SMILES: FC(F)(F)Oc1ccnc(-c2ccc(CBr)cc2)c1]

The title compound was prepared from 2-(p-tolyl)-4-(trifluoromethoxy)pyridine (355 mg, 1.37 mmol) in analogy to general procedure 12 and was obtained as off-white waxy solid (345 mg, 61%). MS (ESI): 333.9 [M+H]⁺

### Intermediate 40

### 2-[4-(bromomethyl)phenyl]-5-(trifluoromethyl)pyrimidine

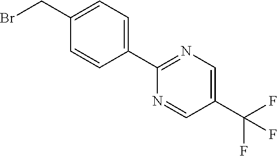 [SMILES: FC(F)(F)c1cnc(-c2ccc(CBr)cc2)nc1]

### Step a) 2-(p-tolyl)-5-(trifluoromethyl)pyrimidine

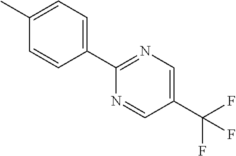 [SMILES: Cc1ccc(-c2ncc(C(F)(F)F)cn2)cc1]

A solution of palladium(II) acetate (12.3 mg, 54.8 μmol, 0.02 eq) THF (8.16 mL) was flushed with Argon and heated to 45° C. under inert atmosphere. Xantphos (60.4 mg, 109.6 μmol, 0.04 eq) was added at that temperature to give a dark green solution, which was stirred for 20 min. 2-Chloro-5-(trifluoromethyl)pyrimidine (500 mg, 2.74 mmol, 1.0 eq), p-tolylboronic acid (446.92 mg, 3.29 mmol, 1.2 eq), K₂CO₃ (757 mg, 5.48 mmol, 2.0 eq) and water (1.63 ml) were added sequentially and the mixture stirred at 45° C. for 4 h. The reaction mixture was partitioned between EtOAC and water. The phases were separated and the aqueous layer was extracted with EtOAC (2×). The combined organic layers were washed with brine, dried over magnesium sulfate and concentrated in vacuo. The remaining crude material was purified by column chromatography on silica gel (0-15% EtOAc in heptane) to afford the title product as white solid (521 mg, 80%). MS (ESI): 239.1 [M+H]⁺

### Step b) 2-[4-(bromomethyl)phenyl]-5-(trifluoromethyl)pyrimidine

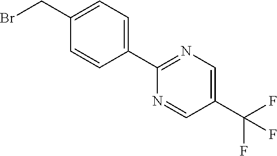 [SMILES: FC(F)(F)c1cnc(-c2ccc(CBr)cc2)nc1]

The title compound was prepared from 2-(p-tolyl)-5-(trifluoromethyl)pyrimidine (521 mg, 2.19 mmol) in analogy to general procedure 12 and was obtained as white solid (576 mg, 68%). MS (ESI): 317.0 [M+H]⁺

### Intermediate 42

### 2-[4-(bromomethyl)phenyl]-5-(1,1,2,2,2-pentafluoroethoxy)pyridine

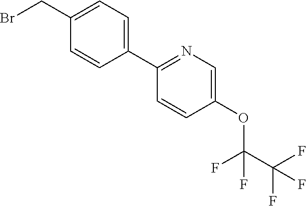 [SMILES: FC(F)(F)C(F)(F)Oc1ccc(-c2ccc(CBr)cc2)nc1]

### Step a) 5-(1,1,2,2,2-pentafluoroethoxy)-2-(p-tolyl)pyridine

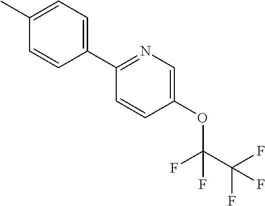 [SMILES: Cc1ccc(-c2ccc(OC(F)(F)C(F)(F)F)cn2)cc1]

2-chloro-5-(1,1,2,2,2-pentafluoroethoxy)pyridine (70 mg, 282.8 μmol, 1.0 eq) was dissolved in 1,4-dioxane (1.41 mL) and water (1.41 mL). p-tolylboronic acid (38.5 mg, 282.77 μmol, 1.0 eq), K₂CO₃ (97.7 mg, 706.9 μmol, 2.5 eq) and 1,1-bis(diphenylphosphino)ferrocene dichloro palladium(II) CH₂Cl₂ adduct (11.55 mg, 14.14 μmol, 0.05 eq) were added to the solution. The reaction was heated to 80° C. and stirred for 90 min. The reaction was poured into water and extracted twice with EtOAc. The organic layers were combined, washed with brine, dried over sodium sulfate and concentrated under reduced pressure. The remaining crude material was purified by column chromatography on silica gel (0-10% EtOAc in heptanes) to afford the title compound as (53.4 mg, 62%) as white solid. MS (ESI): 304.0 [M+H]⁺

### Step b) 2-[4-(bromomethyl)phenyl]-5-(1,1,2,2,2-pentafluoroethoxy)pyridine

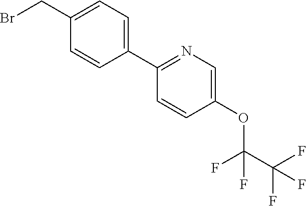 [SMILES: FC(F)(F)C(F)(F)Oc1ccc(-c2ccc(CBr)cc2)nc1]

The title compound was prepared from 5-(1,1,2,2,2-pentafluoroethoxy)-2-(p-tolyl)pyridine (53.4 mg, 176 mol) in analogy to general procedure 12 and was obtained as off-white solid (50 mg, 62% yield). MS (ESI): 381.9 [M+H]⁺

### Intermediate 48

### 5-[4-(bromomethyl)phenyl]-3-(trifluoromethyl)-1,2,4-oxadiazole

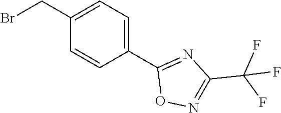 [SMILES: FC(F)(F)c1noc(-c2ccc(CBr)cc2)n1]

### Step a) [(Z)-(1-amino-2,2,2-trifluoro-ethylidene)amino]4-methylbenzoate

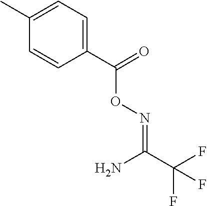 [SMILES: Cc1ccc(C(=O)O/N=C(\N)C(F)(F)F)cc1]

To a solution of 2,2,2-trifluoro-N′-hydroxy-acetamidine (150 mg, 1.17 mmol, 1.0 eq) and DIPEA (379 mg, 2.93 mmol, 2.5 eq) in DCM (3 mL) was added dropwise a solution of 4-methylbenzoyl chloride (190.14 mg, 1.23 mmol, 1.05 eq) in DCM (2 mL) at 0° C. After complete addition, the mixture was stirred at room temperature for 1 h. The reaction mixture was concentrated under reduced pressure. EtOAc (20 mL) and water (10 mL) were added to the remaining residue and the layers were separated. The aqueous phase was extracted with EtOAc (2×). The combined organic extracts were washed with brine, dried over sodium sulfate, filtered, and concentrated under reduced pressure to give the title compound (350 mg, 1.42 mmol, 102% yield) as off-white solid. MS (ESI): 247.1 [M+H]⁺

### Step b) 5-(p-tolyl)-3-(trifluoromethyl)-1,2,4-oxadiazole

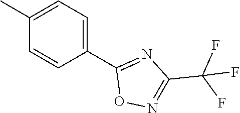 [SMILES: Cc1ccc(-c2nc(C(F)(F)F)no2)cc1]

To a solution of [(Z)-(1-amino-2,2,2-trifluoro-ethylidene)amino]4-methylbenzoate (350 mg, 1.42 mmol, 1.0 eq) in DMSO (4 mL) was added KOH (159.5 mg, 2.84 mmol, 2.0 eq) at room temperature and the mixture was stirred for 1 h. The reaction mixture was poured into water and extracted with EtOAc (3×). The combined extracts were washed with brine, dried over sodium sulfate, filtered, and concentrated under reduced pressure. The remaining residue was purified by preparative TLC (20% EtOAc in petroleum ether) to give 5-(p-tolyl)-3-(trifluoromethyl)-1,2,4-oxadiazole (190 mg, 0.83 mmol, 56% yield) as light yellow solid. MS (ESI): 229.1 [M+H]⁺

### Step c) 5-[4-(bromomethyl)phenyl]-3-(trifluoromethyl)-1,2,4-oxadiazole

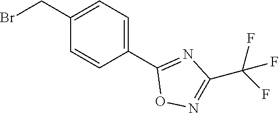 [SMILES: FC(F)(F)c1noc(-c2ccc(CBr)cc2)n1]

The title compound as prepared from 5-(p-tolyl)-3-(trifluoromethyl)-1,2,4-oxadiazole (500 mg, 2.5 mmol) in analogy to general procedure 12 and was obtained as light yellow oil (300 mg, 0.98 mmol, 74% yield). MS (ESI): 307.0 [M+H]⁺

### Intermediate 49

### 3-[4-(bromomethyl)phenyl]-5-(2,2,2-trifluoro-1-methyl-ethyl)-1,2,4-oxadiazole

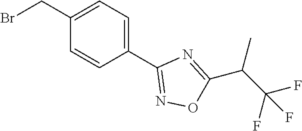 [SMILES: CC(c1nc(-c2ccc(CBr)cc2)no1)C(F)(F)F]

### Step a) [(Z)-[amino(p-tolyl)methylene]amino]3,3,3-trifluoro-2-methyl-propanoate

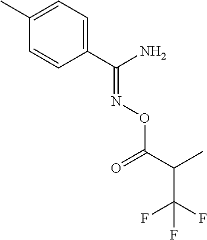 [SMILES: Cc1ccc(/C(N)=N/OC(=O)C(C)C(F)(F)F)cc1]

To a solution of N′-hydroxy-4-methyl-benzamidine (100 mg, 625.9 μmol, 1.0 eq) in THF (3.13 mL) was added HATU (357 mg, 938.87 μmol, 1.5 eq), DIEA (273.3 uL, 1.56 mmol, 2.5 eq) and 3,3,3-trifluoro-2-methyl-propionic acid (82.1 uL, 751.1 μmol, 1.2 eq). The resulting yellow solution was stirred at room temperature for 90 min. The reaction mixture was quenched upon addition of water. The mixture was extracted using EtOAc (3×). The combined organic phases was dried over sodium sulfate and concentrated under reduced pressure. The remaining crude was purified by column chromatography on silica gel (5% MeOH in DCM) to afford the title compound (131 mg, 76%) as white crystalline solid. MS (ESI): 275.2 [M+H]⁺

### Step b) 3-(p-tolyl)-5-(2,2,2-trifluoro-1-methyl-ethyl)-1,2,4-oxadiazole

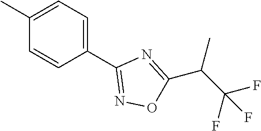 [SMILES: Cc1ccc(-c2noc(C(C)C(F)(F)F)n2)cc1]

[(Z)-[amino(p-tolyl)methylene]amino]3,3,3-trifluoro-2-methyl-propanoate (130.5 mg, 475.86 μmol, 1.0 eq) was suspended in toluene (9.5 ml). The mixture was refluxed for 20 h. The reaction solution was directly purified by column chromatography on silica geld (5-50% DCM in heptanes) to afford 3-(p-tolyl)-5-(2,2,2-trifluoro-1-methyl-ethyl)-1,2,4-oxadiazole (92 mg, 76%) as light yellow oil. MS (ESI): 257.2 [M+H]⁺

### Step c) 3-[4-(bromomethyl)phenyl]-5-(2,2,2-trifluoro-1-methyl-ethyl)-1,2,4-oxadiazole

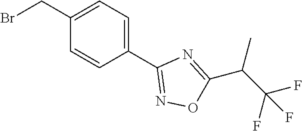 [SMILES: CC(c1nc(-c2ccc(CBr)cc2)no1)C(F)(F)F]

The title compound was prepared from 3-(p-tolyl)-5-(2,2,2-trifluoro-1-methyl-ethyl)-1,2,4-oxadiazole (107.9 mg, 421 mol) in analogy to general procedure 12 and was obtained as white solid (66.5 mg, 45%). MS (ESI): 335.9 [M+H]⁺

### Intermediate 50

### 2-[4-(chloromethyl)phenyl]-5-(2,2,2-trifluoroethoxy)pyridine

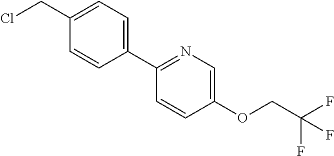 [SMILES: FC(F)(F)COc1ccc(-c2ccc(CCl)cc2)nc1]

### Step a) [4-[5-(2,2,2-trifluoroethoxy)-2-pyridyl]phenyl]methanol

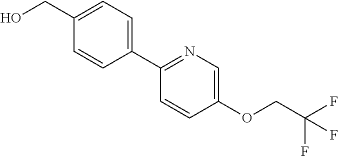 [SMILES: OCc1ccc(-c2ccc(OCC(F)(F)F)cn2)cc1]

To a solution of p-hydroxymethylphenylboronic acid (500 mg, 3.29 mmol, 1.0 eq), 2-bromo-5-(2,2,2-trifluoroethoxy)pyridine (1011 mg, 3.95 mmol, 1.2 eq), and K₂CO₃ (1364.25 mg, 9.87 mmol, 3.0 eq) in water (2 mL) and 1,4-dioxane (16 mL) was added 1,1-bis(diphenylphosphino)ferrocene dichloro palladium(II) CH₂Cl₂ adduct (268 mg, 0.33 mmol, 0.1 eq) under inert conditions. The mixture was stirred at 90° C. for 12 h. The mixture was quenched with water (50 mL) and extracted with EtOAc (100 mL×3). The combined organic phases were washed with brine (200 mL×2), dried over sodium sulfate and concentrated under reduced pressure. The remaining crude was purified by column chromatography on silica gel (0-60% EtOAc in petroleum ether) to afford [4-[5-(2,2,2-trifluoroethoxy)-2-pyridyl]phenyl]methanol (520 mg, 1.84 mmol, 56% yield) as a yellow solid. MS (ESI): 284.1 [M+H]⁺

### Step b) 2-[4-(chloromethyl)phenyl]-5-(2,2,2-trifluoroethoxy)pyridine

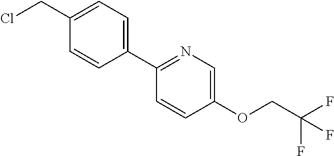 [SMILES: FC(F)(F)COc1ccc(-c2ccc(CCl)cc2)nc1]

To a solution of [4-[5-(2,2,2-trifluoroethoxy)-2-pyridyl]phenyl]methanol (500 mg, 1.77 mmol, 1.0 eq) in DCM (10 mL) was added thionyl chloride (0.38 mL, 5.3 mmol, 3.0 eq) at room temperature and the mixture was stirred for 1 h. The reacting was concentrated under reduced pressure to afford 2-[4-(chloromethyl)phenyl]-5-(2,2,2-trifluoroethoxy)pyridine (510 mg, 1.69 mmol, 96% yield) as a yellow oil, which was used without further purification. MS (ESI): 302.1 [M+H]⁺

### Intermediate 52

### 5-[4-(chloromethyl)phenyl]-2-(trifluoromethoxy)pyridine

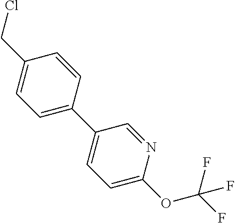 [SMILES: FC(F)(F)Oc1ccc(-c2ccc(CCl)cc2)cn1]

### Step a) [4-[6-(trifluoromethoxy)-3-pyridyl]phenyl]methanol

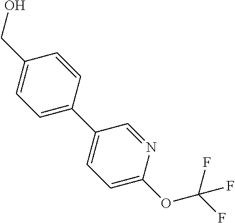 [SMILES: OCc1ccc(-c2ccc(OC(F)(F)F)nc2)cc1]

The solution of [4-[6-(trifluoromethoxy)-3-pyridyl]phenyl]methanol (450 mg, 1.86 mmol, 1.0 eq), 4-(hydroxymethyl)phenylboronic acid (339 mg, 2.23 mmol, 1.2 eq), K₂CO₃ (514 mg, 3.72 mmol, 2.0 eq) and 1,1-bis(diphenylphosphino)ferrocene dichloro palladium(II) CH₂Cl₂ adduct (75.87 mg, 0.09 mmol, 0.05 eq) in 1,4-Dioxane (5 mL) and water (3 mL) was stirred at 90° C. for 12 h under inert atmosphere. The reaction mixture was concentrated under reduced pressure and the remaining residue directly purified by column chromatography on silica gel (25% EtOAc in petroleum ether) to afford the title compound (560 mg, 2.08 mmol, 99% yield) as light yellow solid. MS (ESI): 270.1 [M+H]⁺

### Step b) 5-[4-(chloromethyl)phenyl]-2-(trifluoromethoxy)pyridine

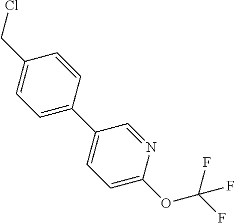 [SMILES: FC(F)(F)Oc1ccc(-c2ccc(CCl)cc2)cn1]

To a solution of [4-[6-(trifluoromethoxy)-3-pyridyl]phenyl]methanol (540 mg, 2.01 mmol, 1.0 eq) in DCM (6 mL) at 0° C. was added dropwise thionyl chloride (0.15 mL, 2.11 mmol, 1.05 eq). After complete addition, the mixture was allowed to warm to room temperature and stirred for 3 h., after the mixture was stirred at 20° C. for 3 h. The reaction mixture was directly concentrated under reduced pressure to afford 5-[4-(chloromethyl)phenyl]-2-(trifluoromethoxy)pyridine (345 mg, 1.2 mmol, 58% yield) as light yellow solid, which was used in the next step without further purification. MS (ESI): 288.1 [M+H]⁺

### Intermediate 53

### 1-[4-(bromomethyl)phenyl]-4-(trifluoromethoxy)pyrazole

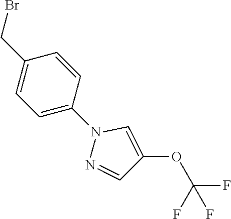 [SMILES: FC(F)(F)Oc1cnn(-c2ccc(CBr)cc2)c1]

### Step a) methoxy-[1-(p-tolyl)pyrazol-4-yl]oxy-methanethione

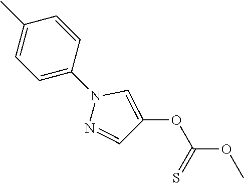

To a solution of 1-(4-methylphenyl)-1H-pyrazol-4-ol (2300 mg, 13.2 mmol, 1.0 eq, CAS 77458-34-5) and methyl 3-methylimidazol-3-ium-1-carbodithioate (3432 mg, 19.8 mmol, 1.5 eq) in MeCN (40 mL) was added triethylamine (3.68 mL, 26.41 mmol, 2.0 eq) at room temperature and the mixture was stirred for 1 h. The mixture was poured into water (40 mL) and the aqueous phase was extracted with EtOAc (30 mL×3). The combined organic phase was washed with brine (100 mL), dried with anhydrous sodium sulfate, filtered and concentrated under reduced pressure. The remaining residue was purified by column chromatography (0-10% EtOAc in petroleum ether) to give methoxy-[1-(p-tolyl)pyrazol-4-yl]oxy-methanethione (2.7 g, 10.21 mmol, 76% yield) as a yellow solid. MS (ESI): 265.1 [M+H]⁺

### Step b) 1-(p-tolyl)-4-(trifluoromethoxy)pyrazole

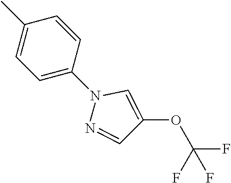 [SMILES: Cc1ccc(-n2cc(OC(F)(F)F)cn2)cc1]

A solution of 1,3-dibromo-5,5-dimethylimidazolidine-2,4-dione (8652 mg, 30.26 mmol, 4.0 eq) and pyridine hydrofluoride (65%, 1165 mg, 7.57 mmol, 1.0 eq) in DCM (15 mL) was stirred at −70° C. for 30 min. The solution was then cooled to −78° C. and a solution of methoxy-[1-(p-tolyl)pyrazol-4-yl]oxy-methanethione (2.0 g, 7.57 mmol, 1.0 eq) in DCM (15 mL) was added drop-wise. After complete addition, the mixture was stirred at 0° C. for 30 min. The reaction was quenched by pouring the mixture in sat. aq. NaHCO₃ (80 mL) and the aqueous phase was extracted with EtOAc (3×60 mL). The combined organic layers were washed with brine (200 mL), dried over anhydrous sodium sulfate, filtered, and concentrated under reduced pressure. The remaining crude material was purified by column chromatography on silica gel (0-10% EtOAc in petroleum ether) to afford a yellow solid (2.15 g). This yellow solid (containing 5-bromo and 3,5-dibromo pyrazole) was dissolved in MeOH (10 ml) and Pd/C (500 mg, 18.69 mmol, 4.8 eq) was added in one portion under argon atmosphere. The reaction mixture was evacuated and backfilled with H₂ three times using a hydrogen balloon. The reaction was then stirred under H₂ atmosphere for 1 h. The resulting black suspension was filtered over celite and concentrated under reduced pressure to afford 1-(p-tolyl)-4-(trifluoromethoxy)pyrazole (1.56 g, 6.44 mmol, 85%) as yellow solid. MS (ESI): 243.3 [M+H]⁺

### Step c) 1-[4-(bromomethyl)phenyl]-4-(trifluoromethoxy)pyrazole

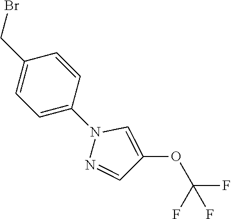 [SMILES: FC(F)(F)Oc1cnn(-c2ccc(CBr)cc2)c1]

The title compound was prepared from 1-(p-tolyl)-4-(trifluoromethoxy)pyrazole (1.56 g, 6.44 mmol, 1.0 eq) in analogy to general procedure 12 and was obtained as yellow solid (1.67 g, 5.2 mmol, 81% yield). MS (ESI): 323.0 [M+H]⁺

### Intermediate 55

### 5,5-difluoro-1-(2-methoxyethyl)piperidine-3-carboxylic acid

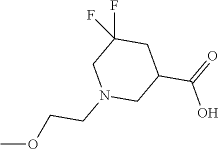

### Step a) methyl 5,5-difluoropiperidine-3-carboxylate hydrochloride

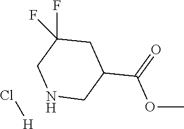

To a solution of 1-tert-butyl 3-methyl 5,5-difluoropiperidine-1,3-dicarboxylate (2000 mg, 7.16 mmol, 1.0 eq) in EtOAc (5 mL) was added HCl in EtOAc (4M, 25 mL, 100 mmol, 14 eq) at room temperature and stirred for 1 h. The reaction mixture was directly concentrated under reduced pressure to afford methyl 5,5-difluoropiperidine-3-carboxylate (1500 mg, 6.96 mmol, 97% yield) as white solid, as hydrochloride salt. MS (ESI): 180.2 [M+H]⁺

### Step b) methyl 5,5-difluoro-1-(2-methoxyethyl)piperidine-3-carboxylate

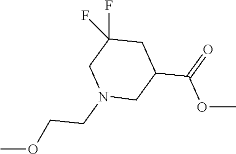

To a solution of methyl 5,5-difluoropiperidine-3-carboxylate hydrochloride (1000 mg, 4.64 mmol, 1.0 eq), 2-bromoethyl methyl ether (1.31 mL, 13.91 mmol, 3.0 eq), potassium carbonate (1922.8 mg, 13.91 mmol, 3.0 eq) in MeCN (20 mL) was added potassium iodide (0.12 mL, 2.32 mmol, 0.5 eq) at room temperature then the mixture was stirred at 80° C. for 12 h. The reaction was poured into water (50 mL). The aqueous phase was extracted with EtOAc (50 mL×3). The combined organic phase was washed with brine (100 mL), dried with anhydrous sodium sulfate, filtered and concentrated in vacuum to give methyl 5,5-difluoro-1-(2-methoxyethyl)piperidine-3-carboxylate (1050 mg, 4.43 mmol, 95% yield) as yellow oil. ¹H-NMR (CDCl3, 400 MHz): δ=3.71 (s, 3H), 3.55-3.49 (m, 2H), 3.35 (s, 3H), 3.22-3.09 (m, 2H), 2.95-2.86 (m, 1H), 2.82-2.65 (m, 2H), 2.48-2.35 (m, 2H), 2.34-2.27 (m, 1H), 1.97-1.80 (m, 1H).

### Step c) 5,5-difluoro-1-(2-methoxyethyl)piperidine-3-carboxylic acid

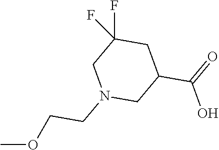

To a solution of methyl 5,5-difluoro-1-(2-methoxyethyl)piperidine-3-carboxylate (600 mg, 2.53 mmol, 1.0 eq) in THF (10 mL) was added a solution of LiOH hydrate (202 mg, 5.06 mmol, 2.0 eq) in water (2 mL) dropwise and the reaction mixture was stirred at room temperature for 2 h. The residue was poured into water (10 mL) and the pH adjusted to pH=5 with 1M HCl. The mixture was lyophilized to give 5,5-difluoro-1-(2-methoxyethyl)piperidine-3-carboxylic acid (550 mg, 2.46 mmol, 97% yield) as white solid. ¹H-NMR (DMSO-d⁶, 400 MHz): δ=3.78-3.74 (m, 4H), 3.51-3.49 (m, 2H), 3.33-3.31 (m, 2H), 3.26 (s, 3H), 3.16-3.12 (m, 1H), 2.45-2.41 (m, 1H), 2.27-2.14 (m, 1H).

### Intermediate 59

### 2-[4-(bromomethyl)phenyl]-4-methyl-5-(trifluoromethoxy)pyridine

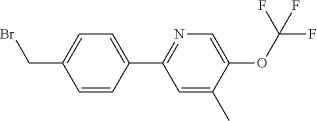 [SMILES: Cc1cc(-c2ccc(CBr)cc2)ncc1OC(F)(F)F]

### Step a) [4-[4-methyl-5-(trifluoromethoxy)-2-pyridyl]phenyl]methanol

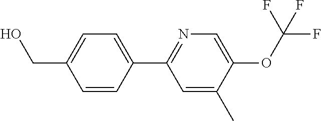 [SMILES: Cc1cc(-c2ccc(CO)cc2)ncc1OC(F)(F)F]

2-chloro-4-methyl-5-(trifluoromethoxy)pyridine (243 mg, 953 μmol, 1.0 eq) was dissolved in 1,4-dioxane (4.77 mL) and water (4.77 mL). 4-(hydroxymethyl)phenylboronic acid (173.8 mg, 1.14 mmol, 1.2 eq), K₂CO₃ (329.39 mg, 2.38 mmol, 2.5 eq) and 1,1-bis(diphenylphosphino)ferrocene dichloro palladium(II) CH₂Cl₂ adduct (38.93 mg, 47.67 μmol, 0.05 eq) were added to the solution. The reaction mixture was heated up to 80° C. and stirred for 1 hour. The reaction mixture was poured into water and extracted twice with EtOAc. The organic layers were combined, washed with brine, dried over sodium sulfate and concentrated under reduced pressure. The remaining crude material was purified by column chromatography on silica gel (0-50% EtOAc in heptane) to afford the title compound (247.3 mg, 78%) as white solid. MS (ESI): 284.0 [M+H]⁺

### Step b) 2-[4-(bromomethyl)phenyl]-4-methyl-5-(trifluoromethoxy)pyridine

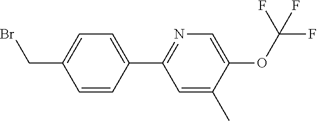 [SMILES: Cc1cc(-c2ccc(CBr)cc2)ncc1OC(F)(F)F]

[4-[4-methyl-5-(trifluoromethoxy)-2-pyridyl]phenyl]methanol (247.3 mg, 742.1 μmol, 1.0 eq) was dissolved in DCM (2.74 mL). Carbon tetrabromide (295.3 mg, 890.5 μmol, 1.2 eq) and Ph₃P (233.6 mg, 890.5 μmol, 1.2 eq) were added to the solution. The reaction mixture was stirred for 90 min at room temperature. The reaction mixture was concentrated completely under reduced pressure and the remaining crude material purified by column chromatography on silica gel (0-30% EtOAc in heptane) to afford the title compound as white solid (127.5 mg, 44%). MS (ESI): 346.0 [M+H]⁺

### Intermediate 60

### 1-[4-(bromomethyl)phenyl]-4-(trifluoromethyl)triazole

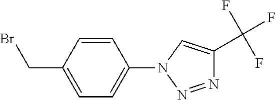 [SMILES: FC(F)(F)c1cn(-c2ccc(CBr)cc2)nn1]

### Step a) 1-(p-tolyl)-4-(trifluoromethyl)triazole

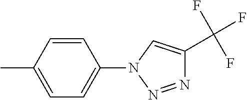 [SMILES: Cc1ccc(-n2cc(C(F)(F)F)nn2)cc1]

A suspension of 4-bromotoluene (2.5 g, 1.8 mL, 14.59 mmol, 2.0 eq), 4-(trifluoromethyl)-1H-triazole (1 g, 7.3 mmol, 1.0 eq), cesium carbonate (7.13 g, 21.9 mmol, 3.0 eq) and N,N′-dimethylethane-1,2-diamine (1.29 g, 1.57 mL, 14.6 mmol, 2.0 eq) in DMF (50 mL) was degassed with Argon for 10 min. Then copper (I) iodide (2.78 g, 14.59 mmol, 2.0 eq) was added at room temperature to give a green suspension. The reaction mixture was heated up to 110° C. and stirred for 3 h. Another batch of 4-bromotoluene (1.25 g, 897.7 uL, 7.3 mmol, 1.0 eq) was added and stirring was continued for 3 h. The reaction mixture cooled to room temperature and partitioned between EtOAc (50 ml) and water (50 ml). The layers were separated and the aqueous layer was extracted with EtOAc. The combined organic layers were washed with brine, dried over anhydrous sodium sulfate and concentrated under reduced pressure. The crude material was purified by column chromatography on silica gel (0-8% EtOAc in heptane) to afford the title compound as white solid (130 mg, 8%). MS (ESI): 228.2 [M+H]⁺

### Step b) 1-[4-(bromomethyl)phenyl]-4-(trifluoromethyl)triazole

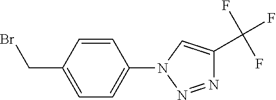 [SMILES: FC(F)(F)c1cn(-c2ccc(CBr)cc2)nn1]

The title compound was prepared from 1-(p-tolyl)-4-(trifluoromethyl)triazole (150 mg, 627 mol) in analogy to general procedure 12 and was obtained as white sold (100 mg, 51%). MS (ESI): 306.0 [M+H]⁺

### Intermediate 63

### 5-[4-(bromomethyl)phenyl]-1-methyl-3-(trifluoromethoxy)pyrazole

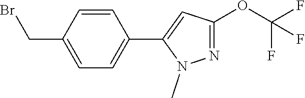 [SMILES: Cn1nc(OC(F)(F)F)cc1-c1ccc(CBr)cc1]

### Step a) 3-[bromo(difluoro)methoxy]-1-methyl-5-(p-tolyl)pyrazole

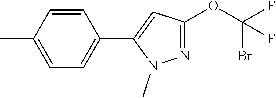 [SMILES: Cc1ccc(-c2cc(OC(F)(F)Br)nn2C)cc1]

To a solution of 1-methyl-5-(p-tolyl)pyrazol-3-ol (1000 mg, 5.31 mmol, 1.0 eq, CAS 199587-27-4) and tetrabutylammonium bromide (0.16 mL, 0.53 mmol, 0.1 eq) in DMF (10 mL) was added NaH (424.97 mg, 10.63 mmol, 2.0 eq) at room temperature. After stirring for 30 min, the mixture was stirred cooled to −30° C. and dibromodifluoromethane (5574 mg, 26.56 mmol, 5.0 eq) was added dropwise. After complete addition, the reaction mixture was allowed to warm to room temperature over 2 hours. The mixture was carefully warmed and stirred at 35° C. for 2 h. The solution was poured into water and extracted with EtOAc (2×). The combined organic phases were washed with brine, dried over sodium sulfate and concentrated under reduced pressure. The remaining residue was purified by column chromatography on silica gel (5-50% EtOAc in petroleum ether) to give the title product (130 mg, 0.41 mmol, 5% yield) as a light yellow oil. MS (ESI): 319.0 [M+H]⁺

### Step b) 1-methyl-5-(p-tolyl)-3-(trifluoromethoxy)pyrazole

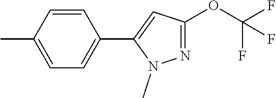 [SMILES: Cc1ccc(-c2cc(OC(F)(F)F)nn2C)cc1]

To a solution of 3-[bromo(difluoro)methoxy]-1-methyl-5-(p-tolyl)pyrazole (130 mg, 0.41 mmol, 1.0 eq) in DCM (5 ml) was added AgBF₄ (160 mg, 0.82 mmol, 2.0 eq) at room temperature and stirred for 1 h. The reaction mixture was filtered and the filtrate concentrated under reduced pressure. The remaining crude material was purified by column chromatography on silica gel (20% EtOAc in petroleum ether) to afford the title product (70 mg, 0.27 mmol, 54% yield) as colorless oil. MS (ESI): 257.0 [M+H]⁺

### Step c) 5-[4-(bromomethyl)phenyl]-1-methyl-3-(trifluoromethoxy)pyrazole

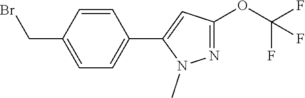 [SMILES: Cn1nc(OC(F)(F)F)cc1-c1ccc(CBr)cc1]

The title compound was prepared from 1-methyl-5-(p-tolyl)-3-(trifluoromethoxy)pyrazole (63 mg, 0.25 mmol) in analogy to general procedure 12 and was obtained as yellow oil (40 mg, 0.12 mmol, 49% yield). MS (ESI): 335.0 [M+H]⁺

### 2) Biological Examples

### 2.1) In Vitro DGK Inhibition Assays

DGKα and ζ kinases use ATP to phosphorylate the substrate 1,2-dilauroyl-sn-glycerol (DLG, incorporated in the liposomes). ATP is converted to ADP as a result of this enzymatic reaction. After the kinase reaction, an ATP-depletion reagent is added to terminate the kinase reaction and deplete any remaining ATP, leaving only ADP. Second, a detection reagent is added to simultaneously convert ADP to ATP and allow the newly synthesized ATP to be converted to light using a coupled luciferase/luciferin reaction.

### Reagents and Material

### Buffer Ingredients (Solutions & Salts)

| Chemicals | Vendor | Cat. Nr.: |
|---|---|---|
| DTT | AppliChem | Cat# A3668 |
| BSA | Sigma - Aldrich | Cat# A2153-10G |
| MOPS | Sigma - Aldrich | Cat# M1254 |
| Sodium chloride (NaCl) | Sigma - Aldrich | Cat# S7663-1KG |
| Magnesium chloride (MgCl₂) | MERCK | Cat# 1.05833.0250 |
| Calcium chloride (CaCl₂) | Sigma - Aldrich | Cat# C4901-500G |

### Protein/Substrates/Tracer

| Proteins / Substrate / Tracer | Vendor | Lot#; Probe# |
|---|---|---|
| DGKA | In house production | hDGKalpha(1-735)-N-His |
| DGKZ | In house production | hDGKzeta(1-928)-N-His |
| 18:1 PS (DOPS) | Sigma - Aldrich | Cat# 840035C |
| 16:0-18:1 PC | Sigma - Aldrich | Cat# 850457C |
| 1,2-dilauroyl-sn-glycerol (DLG) | Sigma - Aldrich | Cat# 800812C |
| Ultra Pure ATP | Promega | Cat# V9101 Part No. V915A |
| ADP-Glo Reagent | Promega | Cat# V9101 Part No. V912A |
| Kinase Detection Substr. | Promega | Cat# V9101 Part No. V914A |
| Kinase Detection Buffer | Promega | Cat# V9101 Part No. V913A |

Full length DGK α and ζ were expressed in Sf21 insect cells by infecting the cells with the baculovirus stock at MOI of 2. Purification of both enzymes was performed as previously described by Takahashi et al., PeerJ, 2018 (Takahashi, D.; Sakane, F. Expression and purification of human diacylglycerol kinase alpha from baculovirus-infected insect cells for structural studies. PeerJ2018, 6, No. e5449).

### Hardware

| Material | Vendor | Cat. Nr. / Volume range |
|---|---|---|
| 384 multiwell plate, white | Greiner | Cat# 781904 |
| 384 multiwell plate, white | Corning | Cat# CLS3574 |
| Matrix Multichannel Pipette | Thermo Fisher | 2-125 μl |
| | SCIENTIFIC | |
| Multidrop Combi | Thermo Fisher | 0.5-50 μl/well |
| | SCIENTIFIC | |
| Envision plate reader | Perkin Elmer | |

### Assay Buffer (30 ml)

| | initial | | | | | | |
|---|---|---|---|---|---|---|---|
| | concentration | | Working | | | final Assay | |
| | (Stock) | | Solution (1-time) | | | concentration | |
| | | [mM, | | [mM, | Volume | | [mM, |
| Chemical | conc. | %] | conc. | %] | [μl] | conc. | %] |
| MOPS | 1000 | mM | 50 | mM | 1500 | 50 | mM |
| (pH 7.5) | | | | | | | |
| NaCl | 5000 | mM | 100 | mM | 600 | 100 | mM |
| MgCl₂ | 1000 | mM | 10 | mM | 300 | 10 | mM |
| CaCl₂ | 1 | mM | 0.001 | mM | 30 | 0.001 | mM |
| DTT | 1000 | mM | 1 | mM | 30 | 1 | mM |
| BSA | 10 | % | 0.01 | % | 30 | 0.01 | % |

| | | |
|---|---|---|
| filled up with: | ddWater: | 27510 |

### Assay Procedure

A concentrated liposome solution was prepared in assay buffer without DTT and BSA: 2 mM of DLG in 21 mM of total liposome (2 mM DLG/8 mM PS/11 mM PC). The reaction mixtures contain the assay buffer with a final DLG concentration of 125 uM ATP concentrations of 25 μM (for DGKA assay) or 50 μM (for DGKZ assay). The reactions were started by addition of DGK α and ζ kinases at 4 nM and 2 nM final concentrations, respectively. After 1 hour reaction, the amount of ADP formed was detected with the ADP-Glo kinase assay (Promega) according to the manufacturer instructions. Compounds were added in 11-points dose response, starting at 10 mM, 1:3 dilutions, with a final DMSO concentration of 2%. The multidrop combi was used as a liquid handler and luminescence was read with 0.5 s by the envision reader (PE).

### Results

| | DGKalpha ADP Glo assay | DGKzeta ADP Glo assay |
|---|---|---|
| Example | IC₅₀ (μM) | IC₅₀ (μM) |
| 21 | 16.799 | 2.31 |
| 20 | 4.956 | 1.239 |
| 23 | 1.53 | 0.523 |
| 1 | 2.356 | 0.488 |
| 2 | 0.613 | 0.128 |
| 6 | 1.866 | 0.696 |
| 35 | 10.064 | 3.056 |
| 34 | 12.167 | 1.672 |
| 5 | 0.635 | 0.068 |
| 4 | 3.414 | 0.127 |
| 36 | 2.604 | 0.956 |
| 37 | 2.001 | 8.828 |
| 3 | 10.269 | 1.281 |
| 24 | 9.863 | 1.438 |
| 9 | 3.288 | 0.468 |
| 10 | 5.161 | 0.282 |
| 8 | 7.029 | 0.137 |
| 18 | 3.87 | 0.151 |
| 56 | 1.985 | 0.228 |
| 50 | 3.382 | 0.851 |
| 51 | 6.263 | 1.802 |
| 57 | 3.343 | 2.344 |
| 11 | >6.25200 | 0.69 |
| 32 | 39.35 | 1.743 |
| 14 | 36.868 | 0.144 |
| 19 | 18.185 | 0.783 |
| 22 | 1.864 | 1.089 |
| 13 | >62.49997 | 0.769 |
| 12 | 3.037 | 0.169 |
| 17 | 3.205 | 0.464 |
| 16 | 1.736 | 0.281 |
| 58 | 26.366 | 2.141 |
| 54 | 12.575 | 1.382 |
| 64 | 1.958 | 2.789 |
| 45 | 2.086 | 0.467 |
| 45 | 27.72 | 3.209 |
| 38 | 9.774 | 1.145 |
| 80 | 11.813 | 1.261 |
| 39 | 19.008 | 1.78 |
| 43 | 17.056 | 1.093 |
| 42 | 30.4 | 8.125 |
| 68 | 2.645 | 0.625 |
| 62 | >19.8 | 1.502 |
| 41 | 30.647 | 3.235 |
| 74 | 3.664 | 0.77 |
| 75 | 45.743 | 2.682 |
| 73 | >62.5 | 13.638 |
| 77 | 48.173 | 1.532 |
| 78 | 43.304 | 2.533 |
| 79 | 39.726 | 0.88 |
| 46 | 16.226 | 1.265 |
| 47 | 7.284 | 0.842 |
| 65 | 2.49 | 6.444 |
| 15 | 40.318 | 0.466 |
| 7 | >62.5 | 1.396 |
| 49 | 17.764 | 3.097 |
| 83 | 29.977 | 1.113 |
| 66 | 14.939 | 6.612 |
| 72 | >62.49997 | 20.896 |
| 85 | >62.49997 | 2.097 |
| 86 | >62.5 | 0.642 |
| 87 | >62.5 | 2.314 |
| 59 | 3.039 | 1.14 |
| 60 | 3.633 | 3.078 |
| 53 | 43.351 | 2.421 |
| 52 | 25.16 | 2.317 |
| 76 | 26.088 | 1.084 |
| 25 | 3.544 | 1.273 |
| 81 | 38.851 | 2.45 |
| 63 | 33.722 | 1.715 |
| 84 | >62.5 | 3.066 |
| 27 | 2.948 | 0.758 |
| 28 | 37.311 | 4.264 |
| 55 | >62.5 | 20.31 |
| 48 | >62.5 | 1.588 |
| 71 | 24.718 | 0.895 |
| 33 | 23.755 | 2.639 |
| 164 | >62.49997 | 2.03 |
| 163 | >62.49997 | 10.637 |
| 98 | 23.825 | 1.034 |
| 161 | >62.49997 | 1.915 |
| 89 | 8.266 | 0.613 |
| 182 | >62.49997 | 2.001 |
| 183 | >62.49997 | 0.97 |
| 97 | 4.685 | 2.082 |
| 181 | >62.49997 | 0.2 |
| 150 | >19.77000 | 9.913 |
| 91 | 4.241 | 3.014 |
| 159 | 1.949 | 1.352 |
| 149 | >62.49997 | 4.253 |
| 157 | 41.395 | 2.277 |
| 166 | *3.0295298 | 1.719 |
| 121 | >6.25200 | 0.767 |
| 169 | >19.77000 | 1.768 |
| 167 | >62.49997 | 0.924 |
| 124 | 2.397 | 0.801 |
| 162 | >62.49997 | 1.095 |
| 92 | 17.478 | 1.711 |
| 170 | >62.49997 | 2 |
| 158 | >59.99997 | 1.126 |
| 109 | 2.107 | 0.864 |
| 100 | >62.49997 | 1.464 |
| 116 | >62.49997 | 0.918 |
| 113 | >62.49997 | 1.13 |
| 111 | >62.49997 | 1.125 |
| 168 | >62.49997 | 1.597 |
| 125 | 3.519 | 0.93 |
| 171 | >59.99997 | 0.722 |
| 127 | >59.99997 | 2.446 |
| 96 | *24.616715 | 3.381 |
| 90 | 23.908 | 2.138 |
| 152 | >59.99997 | 12.202 |
| 94 | >59.99997 | 9.188 |
| 88 | 0.762 | 0.183 |
| 154 | 23.64 | 3.101 |
| 172 | 2.52 | 0.732 |
| 95 | 18.982 | 2.419 |
| 93 | 0.884 | 0.485 |
| 153 | 2.048 | 2.251 |
| 126 | 4.382 | 0.541 |
| 110 | >59.99997 | 0.507 |
| 165 | 1.145 | 2.223 |
| 102 | 1.124 | 0.747 |
| 108 | 1.388 | 0.939 |
| 120 | 13.472 | 2.017 |
| 114 | >59.99997 | 3.724 |
| 107 | 1.3 | 0.843 |
| 160 | 5.185 | 0.922 |
| 156 | 17.815 | 2.956 |
| 106 | 14.399 | 2.367 |
| 119 | 3.452 | 1.629 |
| 123 | 1.863 | 0.893 |
| 105 | 0.694 | 0.787 |
| 117 | 1.137 | 1.009 |
| 112 | 1.746 | 0.473 |
| 101 | 25.598 | 0.192 |
| 115 | 0.585 | 0.803 |
| 118 | 16.405 | 0.814 |
| 175 | 0.961 | 0.734 |
| 129 | 1.676 | 0.948 |
| 135 | 1.734 | 0.786 |
| 173 | 2.056 | 0.416 |
| 174 | 9.297 | 1.142 |
| 137 | 11.181 | 0.66 |
| 138 | 0.657 | 0.465 |
| 176 | 1.528 | 1.549 |
| 178 | 7.098 | 1.686 |
| 144 | 15.46 | 0.86 |
| 145 | 18.021 | 0.955 |
| 148 | 0.737 | 1.051 |

In vitro DGK Inhibition Assays (ADP Glo)

DGK α and ζ kinases use ATP to phosphorylate the substrate 1,2-dilauroyl-sn-glycerol (DLG). ATP is converted to ADP as a result of this enzymatic reaction.

After the kinase reaction, an ATP-depletion reagent is added to terminate the kinase reaction and deplete any remaining ATP, leaving only ADP. Second, a detection reagent is added to simultaneously convert ADP to ATP and allow the newly synthesized ATP to be converted to light using a coupled luciferase/luciferin reaction.

### Experimental Procedure, Reagents and Material

DGK α and ζ kinase ADP Glo assays were ran by Reaction Biology Corp., 1 Great Valley Parkway, Suite 2, Malvern, 19355, PA, USA. Information provided by the service provider are the following: DGK α and ζ kinases were used at 2 nM final concentration. Reactions were carried out at 50 M ATP. 500 uM of the substrate DLG (Dilauroyl-sn-glycerol) was used. Compounds were received at 10 mM DMSO stock solution and were tested in 10-dose IC50 duplicate with 3-fold serial dilution starting at 1 μM. A control compound, Calphostin C, was tested in 10-dose IC50 with 3-fold serial dilution starting at 100 μM.

| | DGKalpha | DGKzeta |
|---|---|---|
| Example | IC₅₀ (nM) | IC₅₀ (nM) |
| 20 | 1414 | 438.9 |
| 36 | 58.5 | 50.7 |
| 24 | 282.3 | 509.5 |
| 32 | 173.5 | 128.3 |
| 39 | 94.6 | 56.4 |
| 78 | 105.4 | 25.4 |
| 47 | 238.2 | 71.3 |
| 49 | 227.4 | 364.5 |
| 53 | 160.1 | 92.8 |
| 52 | 63.6 | 101.2 |
| 81 | 225.7 | 53.5 |
| 124 | 23.3 | 56.7 |
| 96 | 44.7 | 107.2 |
| 88 | 11.1 | 4.1 |

### 2.2) IL2 Secretion Measurements

As readouts for T-cell activation, IL2 secretion after 24 hours and proliferation after 5 days was measured. Increases in IL2 secretion and proliferation upon compound treatment were assessed as the % of the maximum of reference compound A1. WO 2016/139181 discloses reference compound A1 as example 70. As a counter screen and to make sure that no unwanted TCR-independent activation was triggered, PBS conditions were run for all compounds.

### Reagents and Material

| Reagents | Information | Stock conc. | | End conc. |
|---|---|---|---|---|
| Compound dilution Buffer | Sigma, #D2650 | >99% | | 0.2% DMSO |
| (CDB) DMSO | | | | |
| RPMI + GlutaMAX | Gibco, #61870-010 | | | |
| Human serum (HS) | Sigma, #H3667-100ML | 100% | | 5% |
| Sodium Pyruvate (100 mM) | Gibco, #11360-039 | 100x | | 1x |
| 2-Mercaptoethanol | Gibco, #31350-010 | 50 | mM | 50 μM |
| Penicillin-Streptomycin | Life Technologies, #15140122 | 100X | | 1X |
| (10,000 U/mL) | | | | |
| Primary T Lymphocyte cells | Buffy coats for PBMCs | | | 312′500 |
| enriched from isolated | ordered from | | | cells/cm2 in 96- |
| Peripheral blood | Blutspendezentrum Basel- | | | well (100k/well) |
| mononuclear cells (PBMCs) | Stadt, RNCB ID: CL008438 | | | |
| Activator Control Ref cpd | reference compound A1, stock | 10 | mM | 20 μM |
| | in DMSO, 0.2% DMSO final | | | |
| Neutral Control DMSO | Sigma, #D2650 | 100% | | 0.2% DMSO |
| Compounds | Stock in DMSO | 10 | mM | DR starting at |
| | | | | 20 μM; Dil. |
| | | | | Factor 3.333; 5 |
| | | | | steps |
| No stimulation Control | PBS++, Gibco #14040-091 | 100% | | 100% |
| PBS++ | | | | |
| Stimulation Control CD3 | CD3 Monoclonal Antibody | 1 | mg/ml | Depending on |

| | | | |
|---|---|---|---|
| | (OKT3), Thermo Fisher | | donor (0.1- |
| | #16-0037-81 | | 1.0 ug/ml) |
| 96-well cell culture plates, | Corning, #354640 | | |
| Poly-D lysine coated | | | |
| Coating buffer for stim | PBS++, Gibco #14040-091 | | |
| control CD3 | | | |
| Washing buffer for coated | PBS−−, Gibco # 14190-094 | | |
| plates and ELISA | | | |
| IL-2 Human ProQuantum | Invitrogen, #A35603 | | |
| Immunoassay Kit | | | |
| MicroAmp ™ EnduraPlate ™ | Applied Biosystems, | | |
| Optical 384-Well for | #4483273 | | |
| immunoassay | | | |
| LightCycler ® 480 Sealing | Roche, #04729757001 | | |
| Foil for immunoassay | | | |
| IL-2 Human Uncoated | Thermo Fisher Scientific, | | |
| ELISA Kit | #88-7025-88 | | |
| Tween 10% for ELISA | Bio-Rad, #161-0781 | 10% | 0.05% |
| Nunc MaxiSorp plates for | Thermo Fisher Scientific, | | |
| ELISA | #439454 | | |
| V-bottom plates for ELISA | Greiner Bio-One, #651201 | | |
| dilutions | | | |
| MACS filter (cell strainer) | Miltenyi Biotech, #130-041- | | |
| | 407 | | |
| TopSeal-A Plus (sealing for | Perkin Elmer, #6050185 | | |
| sn collection) | | | |
| U-bottom plates for IL-2/ | Costar, #3799 | | |
| supernatant harvest | | | |
| LightCycler ® 480 Multiwell | Roche, #04729692001 | | |
| Plate 96 white, ProQuantum | | | |
| working plate | | | |

### Cell Culture

Expanded primary human T-cells were thawed and cultured in RPMI 1640 (Gibco, #61870-010)+5% human serum (HS, Sigma, #H3667)+1 mM Sodium Pyruvate (Gibco, #11360-039)+50 μM 2-mercaptoethanol (Gibco, #31350-010) and 1× Pen-Strep (Life Technologies, #15140122) medium at density of 2 Mio/ml for 3 hours in 5% CO2, 37° C. and 95% humidity. For coating of plates, PBS++ with PBS−− or PBS++ with CD3 antibody (concentration depending on donor and determined by CD3 titrations) was added 100 μl/well to Poly-D Lysine coated 96-well plates. Plates were sealed and incubated at room temperature for 3 hours on a table-top rocking platform. After incubation, plates were washed once with PBS−− and filled with 40 μl/well culture medium only. Compounds were then added (see next section) to medium only plates. After 3 hours of culturing the T-cells, cells were filtered through a cell strainer (Miltenyi Biotech, #130-041-407), counted again and concentration was adjusted to 1.25 Mio/ml.

Cells were then seeded 80 μl/well to the 40 μl/well including dispensed compounds according to plate layout. By adding cells, compounds were further diluted 1:3, and resulting in 100 k cells/120 μl/well. After 24 hours 40 μl of supernatant was collected carefully from the top while not disturbing the cells and transferred into a round bottom 96well plate. Collected and frozen supernatant was used for detection of IL2 using the IL-2 Human ProQuantum Immunoassay Kit (Invitrogen) or using the Human IL-2 ELISA Kit (Thermo Fisher).

Compound Treatment Compounds were added in a 5 or 6pt dose response with the Tecan D300e Digital Dispenser, all conditions 3 times more concentrated than the end-concentration, since cells are added afterwards (80 μl cells to 40 μl prepared medium with treatment). The DR was starting at 20 μM or 10 μM final top concentration and a dilution factor of 3.333. The positive control was the reference compound A1 that was added in a dose response as well, additionally to 3 wells of only 20 μM representing the positive stimulator control. All wells were normalized with DMSO to a final concentration of 0.6% (0.2% end-concentration).

### IL2 ProQuantum Immunoassay

The immunoassay is done following the manufacturer's manual (Invitrogen, #A35603). Additional information: For the immunoassay, MicroAmp™ EnduraPlate™ Optical 384-Well plates are used. Frozen supernatant is thawed and centrifuged for 5 minutes at 1000×g, both steps at 4° C. After centrifugation, required sample amount is taken from the top, and in a separate LightCycler V-bottom plate (working plate) diluted with assay dilution buffer, dilution factor depending on the PBS or CD3 condition but at least 1:3. IL-2 standard and blanks are prepared in the same V-bottom plate, standard with a range of 0.0128-5000 pg/ml (extended version). After preparation, 5 μl of sample dilutions or standard/blanks are transferred to the optical 384-well plate (assay plate) and the 10 μl reaction protocol is being followed. For measurement, the QuantStudio 12K Flex system is used. Raw data is extracted and IL-2 concentrations are calculated with the Thermo Fisher online app (apps.thermofisher.com/apps/proquantum).

### IL2 Elisa

ELISA is done following the manufacturer's manual (Thermo Fisher Scientific, #88-7025-88). Additional information: For the ELISA Nunc MaxiSorp 96we11 plates are used. Frozen supernatant is thawed and centrifuged for 5 minutes at 1000×g, both steps at 4° C. After that, required sample amount is taken from the top, and in a separate V-bottom plate diluted with ELISA diluent, dilution factor depending on the PBS or CD3 condition. IL-2 standard and blanks are prepared in the same V-bottom plate. After preparation, 50 μl of sample dilutions and 100 μl of standard or blanks are transferred to the Nunc plates.

Calculations and Data Reporting CD3 and PBS plates were analysed separately in Genedata Screener using Roche Normalization PCT_POS_CTRL with DMSO set as Neutral Control and 20 μM of the reference compound A1 set as Stimulator Control/100%.

For CD3 conditions EC50 and Emax of the fitted sigmoidal curve were reported. If no curve could be fitted, the EC50 was reported as blank field and the Emax was based on individual data points. The Emax did not always correspond to the highest concentration tested. Compounds which activate unstimulated cells or compounds which negatively affected viability (see proliferation assay) were flagged.

### Results

| | Tcell IL2 CD3 | Eₘₐₓ IL2 |
|---|---|---|
| Example | EC₅₀ (nM) | (% activation) |
| 21 | 8,012.7 | 53 |
| 20 | 7,629.9 | 82 |
| 23 | 769.4 | 120 |
| 1 | 1,310.1 | 91 |
| 2 | 891.4 | 168 |
| 6 | 1,174.1 | 212 |
| 35 | 2,151.0 | 193 |
| 34 | 4,900.9 | 69 |
| 5 | 322.7 | 537 |
| 4 | 207.0 | 220 |
| 36 | 616.0 | 223 |
| 37 | 338.4 | 198 |
| 3 | 213.4 | 594 |
| 24 | 1,056.6 | 131 |
| 9 | 1,104.7 | 558 |
| 10 | 9,430.0 | 123 |
| 8 | 5,603.1 | 96 |
| 18 | 848.8 | 529 |
| 56 | 940.1 | 109 |
| 50 | 3,599.3 | 119 |
| 51 | 2,485.4 | 135 |
| 57 | 651.8 | 100 |
| 11 | 7,696.6 | 64 |
| 32 | 1,923.7 | 321 |
| 14 | 3,987.1 | 93 |
| 19 | >10,000.0 | 40 |
| 22 | 4,579.0 | 75 |
| 13 | >10,000.0 | 26 |
| 12 | 3,052.8 | 167 |
| 17 | 1,535.6 | 221 |
| 16 | 367.8 | 315 |
| 58 | 4,713.2 | 505 |
| 54 | 3,342.5 | 126 |
| 64 | 889.0 | 543 |
| 45 | 840.1 | 179 |
| 45 | >10,000.0 | 25 |
| 38 | 3,114.6 | 311 |
| 80 | 3,541.4 | 360 |
| 39 | 621.9 | 143 |
| 43 | 771.1 | 89 |
| 42 | 1,394.9 | 128 |
| 68 | 1,159.7 | 351 |
| 62 | 2,114.2 | 110 |
| 41 | 3,337.7 | 66 |
| 74 | 1,460.7 | 206 |
| 75 | 2,550.5 | 140 |
| 73 | 2,533.4 | 116 |
| 77 | 3,454.8 | 170 |
| 78 | 2,341.6 | 216 |
| 79 | 2,452.6 | 236 |
| 46 | 330.6 | 263 |
| 47 | 669.7 | 163 |
| 65 | 1,418.4 | 92 |
| 15 | >10,000.0 | 39 |
| 7 | >10,000.0 | 38 |
| 72 | 3,275.9 | 238 |
| 85 | >10,000.0 | 4 |
| 86 | 4,159.8 | 101 |
| 87 | >10,000.0 | 16 |
| 59 | 595.6 | 175 |
| 60 | 424.3 | 163 |
| 53 | 2,066.8 | 187 |
| 52 | 645.1 | 164 |
| 76 | 2,636.0 | 297 |
| 25 | 608.7 | 137 |
| 81 | 522.7 | 66 |
| 63 | 2,409.5 | 78 |
| 69 | 448.3 | 83 |
| 84 | >10,000.0 | 33 |
| 40 | 3,790.1 | 105 |
| 26 | 1,602.4 | 155 |
| 61 | 605.6 | 241 |
| 70 | >10,000.0 | 43 |
| 27 | 476.3 | 160 |
| 28 | 5,012.0 | 81 |
| 55 | 3,113.2 | 74 |
| 48 | 984.0 | 114 |
| 71 | 3,260.9 | 111 |
| 33 | >10,000.0 | 24 |
| 29 | 955.7 | 156 |
| 30 | 3,511.3 | 117 |
| 82 | >10,000.0 | 25 |
| 44 | 1,042.9 | 112 |
| 31 | 906.3 | 133 |
| 67 | 739.2 | 168 |
| 164 | 3,300.5 | 156 |
| 163 | 2,385.3 | 152 |
| 98 | 1,371.4 | 97 |
| 161 | 3,209.9 | 150 |
| 89 | 376.6 | 182 |
| 182 | 3,263.2 | 149 |
| 183 | 6,439.5 | 69 |
| 97 | 6,348.3 | 115 |
| 181 | 6,887.5 | 66 |
| 150 | >10,000.0 | 35 |
| 91 | 3,600.4 | 113 |
| 159 | 912.2 | 188 |
| 149 | 566.7 | 268 |
| 157 | 1,780.1 | 106 |
| 166 | 157.5 | 385 |
| 121 | 732.1 | 207 |
| 169 | 3,166.4 | 128 |
| 167 | 635.4 | 279 |
| 124 | 278.0 | 154 |
| 162 | 1,412.9 | 185 |
| 92 | 1,998.4 | 157 |
| 170 | 8,551.4 | 79 |
| 158 | 1,340.4 | 345 |
| 109 | 3,400.2 | 360 |
| 100 | 3,222.0 | 100 |
| 116 | 1,030.0 | 87 |
| 113 | 1,316.2 | 85 |
| 111 | 1,860.1 | 151 |
| 168 | 4,869.6 | 67 |
| 125 | 4,275.9 | 97 |
| 171 | 3,777.3 | 122 |
| 127 | 4,561.7 | 112 |
| 96 | 1,364.2 | 232 |
| 90 | 1,006.1 | 159 |
| 152 | >10,000.0 | 19 |
| 94 | 2,664.8 | 76 |
| 88 | 263.7 | 121 |
| 154 | 990.8 | 127 |
| 172 | 1,086.6 | 120 |
| 95 | 807.5 | 172 |
| 93 | *202.42734 | 279 |
| 153 | 245.3 | 193 |
| 126 | 2,113.9 | 254 |
| 110 | 4,110.2 | 85 |
| 165 | *1329.0968 | 123 |
| 102 | 635.0 | 143 |
| 108 | 1,559.7 | 245 |
| 120 | 1,328.1 | 135 |
| 151 | *903.3585 | 65 |
| 114 | 1,051.3 | 48 |
| 107 | 1,385.6 | 24 |
| 160 | 6,434.0 | 55 |
| 156 | 2,111.1 | 554 |
| 106 | 3,841.5 | 121 |
| 119 | 1,277.7 | 102 |
| 123 | 1,251.7 | 144 |
| 105 | 999.1 | 146 |
| 117 | 587.3 | 114 |
| 112 | 832.1 | 139 |
| 101 | 3,653.0 | 429 |
| 115 | 506.8 | 112 |
| 118 | 4,069.5 | 172 |
| 175 | >10,000.0 | 52 |
| 129 | 552.5 | 187 |

### 2.3) Proliferation Assay

### Reagents and Material

| Reagents | Information | Stock conc. | | End conc. |
|---|---|---|---|---|
| Compound dilution Buffer | Sigma, #D2650 | >99% | | 0.2% DMSO |
| (CDB) DMSO | | | | |
| RPMI + GlutaMAX | Gibco, #61870-010 | | | |
| Human serum (HS) heat | Sigma, #H3667-100ML | | | 5% |
| inactivated | | | | |
| Sodium Pyruvate (100 mM) | Gibco, #11360-039 | 100x | | 1x |
| 2-Mercaptoethanol | Gibco, #31350-010 | 50 | mM | 50 μM |
| Penicillin-Streptomycin | Life Technologies, #15140122 | 100X | | 1X |
| (10,000 U/mL) | | | | |
| Primary T Lymphocyte cells | Buffy coats for PBMCs | | | 312′500 |
| enriched from isolated | ordered from | | | cells/cm2 in 96- |
| Peripheral blood | Blutspendezentrum Basel- | | | well (100k/well) |
| mononuclear cells (PBMCs) | Stadt, RNCB ID: CL008438 | | | |
| Activator Control Ref cpd | reference compound A1, stock | 10 | mM | 20 μM |
| | in DMSO, 0.2% DMSO final | | | |
| Neutral Control DMSO | Sigma, #D2650 | | | 0.2% DMSO |
| Compounds | Stock in DMSO | 10 | mM | DR starting at |
| | | | | 20 μM; Dil. |
| | | | | Factor 3.333; 5 |
| | | | | steps |
| No stimulation Control | PBS++, Gibco #14040-091 | 100% | | 100% |
| PBS++ | | | | |
| Stimulation Control CD3 | CD3 Monoclonal Antibody | 1 | mg/ml | Depending |

| | | | |
|---|---|---|---|
| | (OKT3), Thermo Fisher | | on donor |
| | #16-0037-81 | | (0.1-1.0 |
| 96-well cell culture plates, | Corning, #354640 | | ug/ml) |
| Poly-D lysine coated | | | |
| Coating buffer for stim | PBS++, Gibco #14040-091 | | |
| control CD3 | | | |
| Washing buffer for coated | PBS−−, Gibco # 14190-094 | | |
| plates | | | |
| CellTiter-Glo ® Luminescent | Promega, #G7572 | 2x | 1x |
| Cell Viability Assay | | | |
| MACS filter (cell strainer) | Miltenyi Biotech, #130-041- | | |
| | 407 | | |
| TopSeal-A Plus (sealing for | Perkin Elmer, #6050185 | | |
| coating) | | | |
| Backing tape (for | Perkin Elmer, #6005199 | | |
| measurement) | | | |

Expanded primary human T-cells are thawed and cultured in RPMI 1640 (Gibco, #61870-010)+5% human serum (HS, Sigma, #H3667)+1 mM Sodium Pyruvate (Gibco, #11360-039)+50 μM 2-mercaptoethanol (Gibco, #31350-010) and 1× Pen-Strep (Life Technologies, #15140122) medium at density of 2 Mio/ml for 3 hours in 5% CO2, 37° C. and 95% humidity. For coating of plates, PBS++ only or PBS++ with CD3 antibody (concentration depending on donor and determined by CD3 titrations) is added 100 μl/well to Poly-D Lysine coated 96-well plates. Plates are sealed and incubated at room temperature for 3 hours on a table-top rocking platform. After incubation, plates are washed once with PBS−− and filled with 40 μl/well culture medium only. Compounds are then added (see next section) to medium only plates. After 3 hours of culturing the T-cells, cells are filtered through a cell strainer (Miltenyi Biotech, #130-041-407), counted again and concentration is adjusted to 1.25 Mio/ml.

Cells are then seeded 80 μl/well to the 40 μl/well including dispensed compounds according to plate layout. By adding cells, compounds are further diluted 1:3, and resulting in 100 k cells/120 μl/well. After 48 hours 40 μl of supernatant is collected carefully from the top while not disturbing the cells. Cells are assessed for proliferation 5 days later by measuring ATP consumption using CellTiterGlo (Promega).

### Compound Treatment

Compounds were added in a 5 or 6pt dose response with the Tecan D300e Digital Dispenser, all conditions 3 times more concentrated than the end-concentration, since cells are added afterwards (80 μl cells to 40 μl prepared medium with treatment). The DR was starting at 20 μM or 10 μM final top concentration and a dilution factor of 3.333. The positive control was the reference compound A1 that was added in a dose response as well, additionally to 3 wells of only 20 μM representing the positive stimulator control. All wells were normalized with DMSO to a final concentration of 0.6% (0.2% end-concentration).

### Cell Titer Glo Measurements

After 5 days, for detection of ATP which is directly proportional to the number of cells present per well, the CellTiter-Glo® 2.0 Reagent is used. After visual control for toxicity or precipitations of the tested compounds, the plates are equilibrated to room temperature for 45 minutes. CellTiter-Glo® 2.0 Reagent is equilibrated to room temperature as well. After equilibration, an equal amount of CellTiter-Glo reagent is added to the cells (80 μl/well) with an electronic multichannel pipette. Plates are placed on a rocking platform for 15 minutes at room temperature. After incubation, the bottom of the plates is sealed with backing tape. Luminescence is measured with PHERAstar FSX (interval time 0.5 sec, gain 3000, focal height 15 mm) and exported as CSV file for analysis in Genedata screener.

### Calculations and Data Reporting

CD3 and PBS plates were analysed separately in Genedata Screener using Roche Normalization PCT_POS_CTRL with DMSO set as Neutral Control and 20 μM of the reference compound A1 set as Stimulator Control/100%.

For CD3 conditions EC50 and Emax of the fitted sigmoidal curve were reported. If no curve could be fitted, the EC50 was reported as blank field and the Emax was based on individual data points. The Emax did not always correspond to the highest concentration tested. Compounds which activate unstimulated cells (see IL2 measurements) or compounds which negatively affected viability were flagged.

### Results

| | Tcell Proliferation CD3 | Eₘₐₓ proliferation |
|---|---|---|
| Example | EC₅₀ (nM) | (% activation) |
| 21 | 7056.9 | 63 |
| 20 | 2383 | 82 |
| 23 | 316.2 | 136 |
| 1 | 843.2 | 73 |
| 2 | 296 | 143 |
| 6 | 765.4 | 109 |
| 35 | 2493.9 | 99 |
| 34 | 1175.2 | 67 |
| 5 | 120.5 | 200 |
| 4 | 83.1 | 207 |
| 36 | 91.7 | 108 |
| 37 | 304 | 173 |
| 3 | 795.4 | 165 |
| 24 | 775.3 | 142 |
| 9 | 506.1 | 163 |
| 10 | 1924.5 | 133 |
| 8 | 3113 | 143 |
| 18 | 444.6 | 275 |
| 56 | 2208.1 | 425 |
| 50 | 1035 | 219 |
| 51 | 882.5 | 112 |
| 57 | 567.3 | 181 |
| 11 | 1870.6 | 136 |
| 32 | 468.1 | 153 |
| 14 | 3854.2 | 96 |
| 19 | 3608.1 | 100 |
| 22 | 3931.1 | 69 |
| 13 | 1082.7 | 84 |
| 12 | 3875.2 | 125 |
| 17 | 50.1 | 167 |
| 16 | 115.3 | 228 |
| 58 | 531.3 | 114 |
| 54 | 891.9 | 94 |
| 64 | 20.8 | 132 |
| 45 | 62.1 | 104 |
| 45 | 226.4 | 34 |
| 38 | 640.5 | 97 |
| 80 | 402.4 | 130 |
| 39 | 180.9 | 91 |
| 43 | 284.2 | 64 |
| 42 | 123.2 | 66 |
| 68 | 26.1 | 104 |
| 62 | 1565.8 | 101 |
| 41 | 902.4 | 85 |
| 74 | 526.7 | 148 |
| 75 | 1202.8 | 115 |
| 73 | 828.1 | 95 |
| 77 | 3925 | 58 |
| 78 | 1092.7 | 150 |
| 79 | 988.9 | 167 |
| 46 | 115.8 | 94 |
| 47 | 459.3 | 159 |
| 65 | 681 | 102 |
| 15 | 3195.5 | 64 |
| 7 | 3523.3 | 97 |
| 49 | 121.6 | 86 |
| 83 | 1007.8 | 99 |
| 66 | 1289.8 | 112 |
| 72 | 1646.5 | 252 |
| 85 | 8169.1 | 62 |
| 86 | 1660.5 | 99 |
| 87 | 3623.2 | 106 |
| 59 | 29 | 103 |
| 60 | 41.7 | 110 |
| 53 | 2221.9 | 120 |
| 52 | 195.8 | 126 |
| 76 | 1363.5 | 263 |
| 25 | 2932.1 | 131 |
| 81 | 728.5 | 138 |
| 63 | 1059.1 | 103 |
| 69 | 408.9 | 125 |
| 84 | 1747.5 | 151 |
| 40 | 2475.5 | 118 |
| 26 | 301.7 | 184 |
| 61 | 216.2 | 228 |
| 70 | 2755.3 | 76 |
| 27 | 251.8 | 212 |
| 28 | 3512 | 108 |
| 55 | 1090.7 | 59 |
| 48 | 320.6 | 139 |
| 71 | 2871.5 | 129 |
| 33 | 3194.6 | 105 |
| 29 | 844.4 | 208 |
| 30 | 4355.2 | 139 |
| 82 | 6123 | 78 |
| 44 | 741 | 124 |
| 31 | 349.4 | 83 |
| 67 | 261.8 | 119 |
| 164 | 390.1 | 86 |
| 163 | 6439.2 | 106 |
| 98 | 1822.6 | 97 |
| 161 | 327.4 | 178 |
| 89 | 202.8 | 153 |
| 182 | 1265.4 | 175 |
| 183 | 1679 | 156 |
| 97 | 375.3 | 96 |
| 181 | 717.5 | 105 |
| 150 | 656.3 | 79 |
| 91 | 404.4 | 127 |
| 159 | 152.1 | 146 |
| 149 | 1205.3 | 118 |
| 157 | 600.4 | 130 |
| 166 | 75.9 | 164 |
| 121 | 1087.4 | 160 |
| 169 | 832.6 | 106 |
| 167 | 629.3 | 186 |
| 124 | 114 | 153 |
| 162 | 562.6 | 186 |
| 92 | 835 | 170 |
| 170 | 1012.4 | 89 |
| 158 | 304.1 | 150 |
| 109 | 445.3 | 129 |
| 100 | 1898.1 | 96 |
| 116 | 439.2 | 97 |
| 113 | 1057.9 | 79 |
| 111 | 926.5 | 117 |
| 168 | 2624.9 | 62 |
| 125 | 2313.9 | 127 |
| 171 | 1516.2 | 109 |
| 127 | 1984.5 | 130 |
| 96 | 174.5 | 103 |
| 90 | 343.6 | 152 |
| 152 | 1437 | 90 |
| 94 | >10,000.0 | 42 |
| 88 | 45.1 | 106 |
| 154 | 984 | 151 |
| 172 | 303.2 | 101 |
| 95 | 1638.7 | 104 |
| 93 | 119.7 | 104 |
| 153 | 130.3 | 178 |
| 126 | 2489.9 | 212 |
| 110 | 5780.8 | 85 |
| 165 | 153.2 | 139 |
| 102 | 363.3 | 127 |
| 108 | 819.7 | 204 |
| 120 | 1520.5 | 205 |
| 151 | 1851.5 | 105 |
| 114 | 7419.1 | 78 |
| 107 | 2163.6 | 165 |
| 160 | 1303.8 | 150 |
| 156 | 1707 | 141 |
| 106 | 3790.8 | 79 |
| 119 | 808.7 | 119 |
| 123 | 859.8 | 103 |
| 105 | 391 | 138 |
| 117 | 174 | 136 |
| 112 | 291.7 | 116 |
| 101 | 552.3 | 175 |
| 115 | 277.5 | 140 |
| 118 | 1571.2 | 144 |
| 175 | 121.1 | 105 |
| 129 | 22.8 | 119 |
| 135 | 256.8 | 149 |
| 173 | 235 | 101 |
| 174 | 543.3 | 124 |
| 137 | 406 | 124 |
| 138 | 83.2 | 158 |
| 176 | 85.7 | 140 |
| 139 | 1017 | 93 |
| 177 | 805.9 | 101 |
| 178 | 133.4 | 96 |
| 179 | 935.9 | 106 |
| 180 | 650.7 | 102 |
| 144 | 291 | 87 |
| 145 | 4262.8 | 86 |
| 148 | 182.4 | 186 |

### 2.4) T-Cell—TCB—MV3 Killing Assays

### Reagents and Material

| Reagents | Information | Stock conc. | End conc. |
|---|---|---|---|
| Compound dilution Buffer | Sigma, #D2650 | >99% | 0.2% DMSO |
| (CDB) DMSO | | | |

MV-3 RFP medium

| | | | |
|---|---|---|---|
| DMEM + GlutaMAX | Gibco, #31966-021 | | |
| Fetal Bovine Serum (FBS) | VWR, #97068-085 | | |
| Penicillin-Streptomycin | Life Technologies, | 100X | 1X |
| (PenStrep) | #15140122 | | |
| (10,000 U/mL) | | | |
| Puromycin dihydrochloride | Sigma, #P9620-10M | | |

T-cell medium

| | | | |
|---|---|---|---|
| RPMI + GlutaMAX | Gibco, #61870-010 | | |
| Human serum (HS) heat | Sigma, #H3667-100ML | | 5% |
| inactivated | | | |
| Sodium Pyruvate (100 mM) | Gibco, #11360-039 | 100x | 1x |
| 2-Mercaptoethanol | Gibco, #31350-010 | 50 mM | 50 μM |
| Penicillin-Streptomycin | Life Technologies, | 100X | 1X |
| (PenStrep) | #15140122 | | |
| (10,000 U/mL) | | | |
| Primary T Lymphocyte cells | PBMCs ordered from | | 312′500 cells/cm2 |
| enriched from isolated | stemcell technologies, | | in 96-well |
| Peripheral blood mononuclear | #70025 | | (100k/well) |
| cells (PBMCs) | | | |
| MV-3 RFP (nuclear | RNCB Specimen ID: | | 31′250 cells/cm2 |
| expression), clone 22 | | | in 96-well |
| | | | (10k/well) |
| Activator Control Ref cpd | reference compound | 10 mM | 20 μM |
| | A1, stock in DMSO, | | |
| | 0.2% DMSO final | | |
| Trypsin 0.05% | Gibco, #25300-054 | | |
| T-flask | Falcon, #353133 | | |
| Neutral Control DMSO | Sigma, #D2650 | | 0.2% DMSO |
| Compounds | Stock in DMSO | 10 mM | DR starting at |
| | | | 20 μM; Dil. Factor |
| | | | 3.333; 8 steps |
| No stimulation Control PBS−− | PBS−−, Gibco # 14190- | 100% | Depending on |
| | 094 | | TCB concentration |
| Stimulation Control MCSP- | In-house generated | Lot | 1.5 pM-5 pM |
| TCB | TCB, Concept ID | dependent | (depending on |
| | P1AD8773-001, WO | | donor sensitivity) |
| | 2014/131711 A1 | | |
| 96-well cell culture plates, | TTP, #92696 | | |
| low evaporation | | | |
| Round bottom plate | Costar, #3799 | | |
| 6-well cell culture plate | Corning, #3506 | | |
| MACS filter (cell strainer) | Miltenyi Biotech, #130- | | |
| | 041-407 | | |

### Cell Culture

All culturing steps are executed at 5% CO2, 37° C. and 95% humidity.

MV-3 RFP cells are cultured in MV-3 medium (DMEM+10% FBS, 1× PenStrep and 0.5 μg/mL Puromycin) for at least 3 weeks. Cultured MV-3 cells at 80% confluency are washed once with PBS−− and trypsinized until detached. Cells are then counted and resuspended to 1*105 cells/mL in T-cell medium (RPMI 1640+5% human serum+1 mM Sodium Pyruvate+50 μM 2-mercaptoethanol and 1× Pen-Strep). Cells are seeded with 100 μL/well into a 96-well plate (TTP, #92696), and placed for 40 minutes without moving at room temperature in order to achieve evenly distributed attachment of cells. Plates are then incubated until further use.

On the next day, expanded primary human T-cells are thawed and resuspended in T-cell medium to 4*106 cells/mL. For 3 hours, they are cultured in a 6-well plate with 6 mL per well at maximum. After culturing the T-cells, they are filtered through a cell strainer (Miltenyi Biotech, #130-041-407), counted again, and cell concentration is adjusted to 2*106 cells/mL.

### Compound Treatment

MCSP-TCB or PBS are pre-diluted in T-cell medium (concentration depending on T-cell donor), 4 times more concentrated than the end-concentration. 60 μL/well of pre-dilutions are then distributed into a round bottom plate (Costar, #3799) according to plate layout. Compounds are added in a 9pt dose response with the Tecan D300e Digital Dispenser, as well 4 times more concentrated than the end-concentration. DMSO concentration of all wells is adjusted to 0.8%, resulting in 0.2% as final concentration.

60 μL per well of T-cell suspension are added to the prepared round bottom plate and resuspended with a manual multichannel. 100 μL/well of the resuspended T-cell suspension including treatments are then transferred cautiously to the over-night cultured MV-3 cells according to plate layout. 100 μL T-cell medium only is added to the outer MV-3 wells only. Final compound DR is starting at 20 μM with a dilution factor of 3.333. Final TCB concentration is between 1.5 pM to 5 pM and was determined for each T-cell donor individually by running TCB titrations. For each donor, a TCB concentration was chosen which corresponds to 10-20% of MV3 baseline cell killing in the absence of compound treatment. Positive control is the reference compound A1 which is added in a DR, as well as additional wells with only 20 μM. 20 μM of reference compound A1 represent the positive stimulator control, TCB only (DMSO wells) the neutral control.

### Calculations

After transfer of T-cells with treatment pre-dilutions, MV-3 cells are imaged by time-lapse microscopy using IncucyteZOOM™ (Essen BioScience, MI, USA). Imaging is performed every 3 hours for a total of 120 hours (10X objective, phase and red image channels, acquisition time 400 ms, Green/Red 4614 optical module). RFP object count per well is analysed in the IncucyteZOOM™ Software (Version 2019B Rev2) with a mask that was previously created and optimized for MV-3 cells. Raw data is exported as object count/well and values are normalized as % TCL compared to wells with MV-3 only, representing 100% growth and therefore 0% TCL.

RFP measurements Calculated % TCL values are analysed in Genedata Screener using Roche Normalization PCT_POS_CTRL with MCSP-TCB only set as Neutral Control and 20 μM of the reference compound A1 set as Stimulator Control/100%.

EC50 and Emax values were provided in the table below.

Induced TCL by compounds without TCB treatment or toxicity (observed in the PBS condition) were be flagged.

### Results

| | MSCP TCB killing | MSCP TCB killing |
|---|---|---|
| | MV-3 cells | MV-3 cells |
| Example | 5 d EC₅₀ (μM) | 5 d Eₘₐₓ (%) |
| 35 | 0.373 | 91 |
| 37 | 0.058 | 133 |
| 24 | 0.273 | 108 |
| 18 | 0.029 | 67 |
| 32 | 0.12 | 100 |
| 54 | 0.246 | 86 |
| 80 | 0.257 | 86 |
| 39 | 0.19 | 72 |
| 78 | 0.093 | 112 |
| 47 | 0.28 | 103 |
| 53 | 0.137 | 98 |
| 52 | 0.052 | 95 |
| 81 | 0.343 | 98 |
| 91 | 0.237 | 96 |
| 159 | 0.166 | 102 |
| 157 | 0.119 | 100 |
| 166 | 0.01 | 102 |
| 121 | 0.109 | 98 |
| 124 | 0.023 | 100 |
| 162 | 0.168 | 100 |
| 92 | 0.12 | 99 |

1. A compound of formula (I) 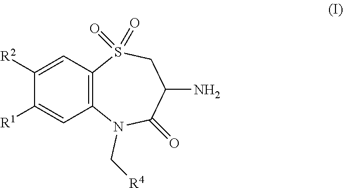
or a pharmaceutically acceptable salt thereof, wherein:
R¹ is oxadiazole, wherein R¹ is optionally substituted with one or more R¹⁰ which can be the same or different;
R² is selected from hydrogen and halogen;
R⁴ is selected from C₅₋₁₄-aryl and 5-14 membered heteroaryl, wherein R⁴ is optionally substituted with one or more R¹¹ which can be the same or different;
R¹⁰ is selected from:
i) C₁₋₁₀-alkyl, optionally substituted with one or more halogen, amino, hydroxy, C₁₋₆-alkoxy, 3-10 membered cycloalkyl, phenyl, cyano;
ii) C₃₋₁₀-cycloalkyl, optionally substituted with one or more halogen, cyano, amino;
iii) 3-10 membered heterocyclyl, optionally substituted with one or more halogen, C₁₋₁₀-alkyl, amino, halo-C₁₋₆-alkyl, hydroxy, cyano, —C(O)O—(R^{10q}), C₃₋₁₀-cycloalkyl, wherein C₁₋₁₀-alkyl is optionally substituted with one or more hydroxy, C₁₋₆-alkoxy;
iv) —N(R^{10e}R^{10f}); and
v) heteroaryl, optionally substituted with one or more C₁₋₁₀-alkyl, halogen;
R^{10e} and R^{10f} are each independently selected from:
i) hydrogen;
ii) C₁₋₆-alkyl, optionally substituted with one or more, cyano, halogen, hydroxy; and
iii) C₃₋₁₀-cycloalkyl, optionally substituted with one or more halogen, C₁₋₁₀-alkyl;
R^{10q} is C₁₋₅-alkyl, wherein C₁₋₅-alkyl is optionally substituted with one or more hydroxy;
R¹¹ is selected from:
i) 5-6 membered heteroaryl, optionally substituted with one or more C₁₋₆-alkyl, C₃₋₁₀ cycloalkyl, halo-C₁₋₆-alkyl, C₁₋₆-alkoxy, halo-C₁₋₆-alkoxy, wherein C₃₋₁₀ cycloalkyl is optionally substituted with one or more halogen; and
ii) phenyl, optionally substituted with one or more C₁₋₆-alkoxy, —OH, halo-C₁₋₆-alkyl.

2. The compound of claim 1, or a pharmaceutically acceptable salt thereof, wherein R¹¹ is selected from:
(i) 5-6 membered heteroaryl, optionally substituted with one or more C₁₋₆-alkyl, C₃₋₁₀ cycloalkyl, halo-C₁₋₆-alkyl; and
(ii) phenyl, optionally substituted with one or more C₁₋₆-alkoxy, halo-C₁₋₆-alkyl, halo-C₁₋₆-alkoxy.

3. The compound of claim 1, or a pharmaceutically acceptable salt thereof, wherein R² is selected from hydrogen and fluorine.

4. The compound of claim 1, or a pharmaceutically acceptable salt thereof, wherein R⁴ is selected from phenyl and pyridinyl, wherein R⁴ is optionally substituted with one or more R¹¹ which can be the same or different.

5. The compound of claim 1, or a pharmaceutically acceptable salt thereof, wherein R¹⁰ is selected from tert-butyl, pyrrolidinyl, tetrafluoro-methoxy-ethyl, methyl-propanenitrile, difluoromorpholinyl, oxa-azaspiro[2.5]octan-yl, (trifluoromethyl)morpholinyl, aminocyclohexyl, cyclopropanecarbonitrile, difluoro-piperidyl, ethoxy-tetrafluoro-ethyl, (hydroxymethyl)tetrahydrofuranyl, azabicyclo[3.1.1]heptane-methylcarboxylate, amino-trifluoromethyl-ethyl, difluoro-piperidine-methylcarboxylate, fluoro-methyl-piperidyl, aminooxetanyl, (difluoro-methyl-cyclobutyl)aminoyl, cyclopropyltetrahydrofuranyl, amino-dimethyl-propyl, propanenitrile, isopropylaminoyl, fluoro-methyl-pyridyl, methyl-pyridyl, chloro-pyridyl, tetrafluoroethyl, trifluoro-dihydroxy-ethyl, hydroxy-(trifluoromethyl)propyl, pentafluoroethyl, trifluoro-dimethyl-ethyl, trifluoro-phenyl-ethyl, benzyl-trifluoroethyl, (trifluoromethyl)oxetanyl, trifluoro(hydroxymethyl)ethyl, amino-cyclopropyl-trifluoro-ethyl, trifluoro-hydroxy-methyl-ethyl, trifluoroethyl, morpholino, hexahydro-2H-pyrano[4,3-b]pyrrolyl, hexahydro-2H-cyclopenta[b][1,4]oxazinyl, dioxaazabicyclo[3.3.1]nonanyl, morpholinyl-carbonitrile, (methoxymethyl)morpholinyl, (hydroxymethyl)morpholinyl, (hydroxyethyl)morpholinyl, oxazepanyl, difluoro-(methoxyethyl)-piperidyl, aminocyclohexyl, amino-trifluoro-methyl-ethyl, methyloxetanyl, trifluoro-hydroxy-(trifluoromethyl)ethyl, (trifluoromethyl)oxetan-3-yl, trifluoro-(hydroxymethyl)ethyl, amino-trifluoro-methyl-ethyl, hexahydrofuro[3,2-b]pyrrolyl, difluoro-azabicyclo[4.1.0]heptanyl, hexahydrofuro[2,3-b][1,4]oxazinyl, hexahydro-2H-cyclopenta[b][1,4]oxazinyl, hexahydro-2H-pyrano[4,3-b][1,4]oxazinyl, hexahydro-2H-cyclopenta[b][1,4]oxazinyl, oxa-azabicyclo[3.2.1]octanyl, cyclopropyl-difluoro-tetrahydrofuranyl, difluorocyclohexyl, amino-trifluoro-ethyl, difluoroethyl(hydroxyethyl)amino, difluoroethyl-aminoyl-acetonitrile, cyclopropyl(difluoroethyl)amino, difluoropyrrolidinyl, (trifluoro-methyl-ethyl)amino, trifluoroethylamino, methyl(trifluoroethyl)amino, ethyl-difluoro-piperidyl, dimethyl-pyridyl, and trifluoro-methoxy-ethyl.

6. The compound of claim 1, or a pharmaceutically acceptable salt thereof, wherein R¹⁰ is selected from tert-butyl, tetrafluoro-methoxy-ethyl, methyl-propanenitrile, difluoromorpholinyl, oxa-azaspiro[2.5]octan-yl, (trifluoromethyl)morpholinyl, cyclopropanecarbonitrile, difluoro-piperidyl, (hydroxymethyl)tetrahydrofuranyl, amino-trifluoromethyl-ethyl, aminooxetanyl, cyclopropyltetrahydrofuranyl, propanenitrile, and aminocyclohexyl.

7. The compound of claim 1, or a pharmaceutically acceptable salt thereof, wherein R¹¹ is selected from (hydroxymethyl)phenyl, (trifluoromethyl)oxadiazolyl, cyclopropyl-oxadiazolyl, (trifluoromethyl)pyridyl, (trifluoromethyl)phenyl, methoxyphenyl, (trifluoromethyl)pyridyl, dimethylpyrazolyl, tert-butyl-oxadiazolyl, methyl-oxadiazolyl, methylpyrazolyl, (difluoromethyl)-oxadiazolyl, (trifluoromethyl)oxazolyl, methyl-(trifluoromethyl)pyrazolyl, (trifluoromethyl)pyrazolyl, (trifluoromethyl)isoxazolyl, (trifluoromethyl-ethyl)oxadiazolyl, (trifluoroethyl)oxadiazolyl, (trifluoromethoxy)phenyl, cyclopropyl-triazolyl, (trifluoromethoxy)pyridyl, (trifluoromethoxy)pyrimidinyl, (pentafluoroethoxy)pyridyl, (trifluoromethoxy), pyridyl, and methyl-(trifluoromethoxy)pyrazolyl.

8. The compound of claim 1, or a pharmaceutically acceptable salt thereof, wherein R¹¹ is selected from (trifluoromethyl)oxadiazolyl, cyclopropyl-oxadiazolyl, (trifluoromethyl)pyridyl, methoxyphenyl, (trifluoromethoxy)pyridyl, (trifluoromethoxy)pyrimidinyl, (pentafluoroethoxy)pyridyl, (trifluoromethoxy), pyridyl, and methyl-(trifluoromethoxy)pyrazolyl.

9. The compound of claim 1, or a pharmaceutically acceptable salt thereof, wherein:
R¹ is oxadiazole, wherein R¹ is optionally substituted with one or more R¹⁰ which can be the same or different;
R² is selected from hydrogen and fluorine;
R⁴ is selected from phenyl and pyridinyl, wherein R⁴ is optionally substituted with one or more R¹¹ which can be the same or different;
R¹⁰ is selected from:
i) C₁₋₁₀-alkyl, optionally substituted with one or more halogen, amino, hydroxy, C₁₋₆-alkoxy, 3-10 membered cycloalkyl, phenyl, cyano;
ii) C₃₋₁₀-cycloalkyl, optionally substituted with one or more halogen, cyano, amino;
iii) 3-10 membered heterocyclyl, optionally substituted with one or more halogen, C₁₋₁₀-alkyl, amino, halo-C₁₋₆-alkyl, hydroxy, cyano, —C(O)O—(R^{10q}), C₃₋₁₀-cycloalkyl, wherein C₁₋₁₀-alkyl is optionally substituted with one or more hydroxy, C₁₋₆-alkoxy;
iv) —N(R^{10e}R^{10f}); and
v) heteroaryl, optionally substituted with one or more C₁₋₁₀-alkyl, halogen;
R^{10e} and R^{10f} are each independently selected from:
i) hydrogen;
ii) C₁₋₆-alkyl, optionally substituted with one or more, cyano, halogen, hydroxy; and
iii) C₃₋₁₀-cycloalkyl, optionally substituted with one or more halogen, C₁₋₁₀-alkyl;
R^{10q} is C₁₋₅-alkyl, wherein C₁₋₅-alkyl is optionally substituted with one or more hydroxy;
R¹¹ is selected from:
i) 5-6 membered heteroaryl, optionally substituted with one or more C₁₋₆-alkyl, C₃₋₁₀ cycloalkyl, halo-C₁₋₆-alkyl; and
ii) phenyl, optionally substituted with one or more C₁₋₆-alkoxy, halo-C₁₋₆-alkyl, halo-C₁₋₆-alkoxy.

10. The compound of claim 1, or a pharmaceutically acceptable salt thereof, wherein:
R¹ is oxadiazole, wherein R¹ is optionally substituted with one or more R¹⁰ which can be the same or different;
R² is selected from hydrogen and fluorine;
R⁴ is selected from phenyl and pyridinyl, wherein R⁴ is optionally substituted with one or more R¹¹ which can be the same or different;
R¹⁰ is selected from tert-butyl, pyrrolidinyl, tetrafluoro-methoxy-ethyl, methyl-propanenitrile, difluoromorpholinyl, oxa-azaspiro[2.5]octan-yl, (trifluoromethyl)morpholinyl, aminocyclohexyl, cyclopropanecarbonitrile, difluoro-piperidyl, ethoxy-tetrafluoro-ethyl, (hydroxymethyl)tetrahydrofuranyl, azabicyclo[3.1.1]heptane-methylcarboxylate, amino-trifluoromethyl-ethyl, difluoro-piperidine-methylcarboxylate, fluoro-methyl-piperidyl, aminooxetanyl, (difluoro-methyl-cyclobutyl)aminoyl, cyclopropyltetrahydrofuranyl, amino-dimethyl-propyl, propanenitrile, isopropylaminoyl, fluoro-methyl-pyridyl, methyl-pyridyl, chloro-pyridyl, tetrafluoroethyl, trifluoro-dihydroxy-ethyl, hydroxy-(trifluoromethyl)propyl, pentafluoroethyl, trifluoro-dimethyl-ethyl, trifluoro-phenyl-ethyl, benzyl-trifluoroethyl, (trifluoromethyl)oxetanyl, trifluoro(hydroxymethyl)ethyl, amino-cyclopropyl-trifluoro-ethyl, trifluoro-hydroxy-methyl-ethyl, trifluoroethyl, morpholino, hexahydro-2H-pyrano[4,3-b]pyrrolyl, hexahydro-2H-cyclopenta[b][1,4]oxazinyl, dioxaazabicyclo[3.3.1]nonanyl, morpholinyl-carbonitrile, (methoxymethyl)morpholinyl, (hydroxymethyl)morpholinyl, (hydroxyethyl)morpholinyl, oxazepanyl, difluoro-(methoxyethyl)-piperidyl, aminocyclohexyl, amino-trifluoro-methyl-ethyl, methyloxetanyl, trifluoro-hydroxy-(trifluoromethyl)ethyl, (trifluoromethyl)oxetan-3-yl, trifluoro-(hydroxymethyl)ethyl, amino-trifluoro-methyl-ethyl, hexahydrofuro[3,2-b]pyrrolyl, difluoro-azabicyclo[4.1.0]heptanyl, hexahydrofuro[2,3-b][1,4]oxazinyl, hexahydro-2H-cyclopenta[b][1,4]oxazinyl, hexahydro-2H-pyrano[4,3-b][1,4]oxazinyl, hexahydro-2H-cyclopenta[b][1,4]oxazinyl, oxa-azabicyclo[3.2.1]octanyl, cyclopropyl-difluoro-tetrahydrofuranyl, difluorocyclohexyl, amino-trifluoro-ethyl, difluoroethyl(hydroxyethyl)amino, difluoroethyl-aminoyl-acetonitrile, cyclopropyl(difluoroethyl)amino, difluoropyrrolidinyl, (trifluoro-methyl-ethyl)amino, trifluoroethylamino, methyl(trifluoroethyl)amino, ethyl-difluoro-piperidyl, dimethyl-pyridyl, and trifluoro-methoxy-ethyl;
R¹¹ is selected from (hydroxymethyl)phenyl, (trifluoromethyl)oxadiazolyl, cyclopropyl-oxadiazolyl, (trifluoromethyl)pyridyl, (trifluoromethyl)phenyl, methoxyphenyl, (trifluoromethyl)pyridyl, dimethylpyrazolyl, tert-butyl-oxadiazolyl, methyl-oxadiazolyl, methylpyrazolyl, (difluoromethyl)-oxadiazolyl, (trifluoromethyl)oxazolyl, methyl-(trifluoromethyl)pyrazolyl, (trifluoromethyl)pyrazolyl, (trifluoromethyl)isoxazolyl, (trifluoromethyl-ethyl)oxadiazolyl, (trifluoroethyl)oxadiazolyl, (trifluoromethoxy)phenyl, cyclopropyl-triazolyl, (trifluoromethoxy)pyridyl, (trifluoromethoxy)pyrimidinyl, (pentafluoroethoxy)pyridyl, (trifluoromethoxy), and pyridyl, methyl-(trifluoromethoxy)pyrazolyl.

11. The compound of claim 1, or a pharmaceutically acceptable salt thereof, wherein:
R¹ is oxadiazole, wherein R¹ is optionally substituted with one or more R¹⁰ which can be the same or different;
R² is selected from hydrogen and fluorine;
R⁴ is selected from phenyl and pyridinyl, wherein R⁴ is optionally substituted with one or more R¹¹ which can be the same or different;
R¹⁰ is selected from tert-butyl, tetrafluoro-methoxy-ethyl, methyl-propanenitrile, difluoromorpholinyl, oxa-azaspiro[2.5]octan-yl, (trifluoromethyl)morpholinyl, cyclopropanecarbonitrile, difluoro-piperidyl, (hydroxymethyl)tetrahydrofuranyl, amino-trifluoromethyl-ethyl, aminooxetanyl, cyclopropyltetrahydrofuranyl, and propanenitrile, aminocyclohexyl;
R¹¹ is selected from (trifluoromethyl)oxadiazolyl, cyclopropyl-oxadiazolyl, (trifluoromethyl)pyridyl, methoxyphenyl, (trifluoromethoxy)pyridyl, (trifluoromethoxy)pyrimidinyl, (pentafluoroethoxy)pyridyl, (trifluoromethoxy), pyridyl, and methyl-(trifluoromethoxy)pyrazolyl.

12. The compound of claim 1, or a pharmaceutically acceptable salt thereof, selected from:
(3R)-3-amino-7-(5-tert-butyl-1,3,4-oxadiazol-2-yl)-8-fluoro-5-[[4-(1-methylpyrazol-3-yl)phenyl]methyl]-1,1-dioxo-2,3-dihydro-1λ⁶,5-benzothiazepin-4-one;
(3R)-3-amino-7-(5-tert-butyl-1,3,4-oxadiazol-2-yl)-8-fluoro-5-[[4-(3-methyl-1,2,4-oxadiazol-5-yl)phenyl]methyl]-1,1-dioxo-2,3-dihydro-1λ⁶,5-benzothiazepin-4-one;
2-[5-[(3R)-3-amino-8-fluoro-1,1,4-trioxo-5-[[4-[5-(trifluoromethyl)-2-pyridyl]phenyl]methyl]-2,3-dihydro-6,5-benzothiazepin-7-yl]-1,3,4-oxadiazol-2-yl]-2-methyl-propanenitrile,
(3R)-3-amino-7-[5-[(3,3-difluoro-1-methyl-cyclobutyl)amino]-1,3,4-oxadiazol-2-yl]-8-fluoro-1,1-dioxo-5-[[4-[5-(trifluoromethyl)-1,2,4-oxadiazol-3-yl]phenyl]methyl]-2,3-dihydro-1λ⁶, 5-benzothiazepin-4-one;
(3R)-3-amino-8-fluoro-7-[5-(4-oxa-7-azaspiro[2.5]octan-7-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-[5-(4,4-difluoro-1-piperidyl)-1,3,4-oxadiazol-2-yl]-8-fluoro-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-8-fluoro-7-[5-(isopropylamino)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one;
(3R)-3-amino-7-(5-tert-butyl-1,2,4-oxadiazol-3-yl)-8-fluoro-5-[[6-[4-(hydroxymethyl)phenyl]-3-pyridyl]methyl]-1,1-dioxo-2,3-dihydro-1λ⁶,5-benzothiazepin-4-one,
(3R)-3-amino-7-[5-(3-aminooxetan-3-yl)-1,2,4-oxadiazol-3-yl]-8-fluoro-5-[[4-(4-methoxyphenyl)phenyl]methyl]-1,1-dioxo-2,3-dihydro-1λ⁶, 5-benzothiazepin-4-one;
(3R)-3-amino-7-[5-(3-aminooxetan-3-yl)-1,2,4-oxadiazol-3-yl]-8-fluoro-1,1-dioxo-5-[[4-[5-(trifluoromethyl)-1,2,4-oxadiazol-3-yl]phenyl]methyl]-2,3-dihydro-1λ⁶,5-benzothiazepin-4-one,
(3R)-3-amino-7-[5-(3-aminooxetan-3-yl)-1,2,4-oxadiazol-3-yl]-8-fluoro-1,1-dioxo-5-[[4-[5-(trifluoromethyl)-2-pyridyl]phenyl]methyl]-2,3-dihydro-1λ⁶, 5-benzothiazepin-4-one;
(3R)-3-amino-8-fluoro-7-[5-(4-oxa-7-azaspiro[2.5]octan-7-yl)-1,2,4-oxadiazol-3-yl]-1,1-dioxo-5-[[6-[4-(trifluoromethyl)phenyl]-3-pyridyl]methyl]-2,3-dihydro-1λ⁶,5-benzothiazepin-4-one;
(3R)-3-amino-7-[5-(2,2-difluoromorpholin-4-yl)-1,2,4-oxadiazol-3-yl]-8-fluoro-1,1-dioxo-5-[[6-[5-(trifluoromethyl)-1,2,4-oxadiazol-3-yl]-3-pyridyl]methyl]-2,3-dihydro-1λ⁶,5-benzothiazepin-4-one,
(3R)-3-amino-7-[5-(2,2-difluoromorpholin-4-yl)-1,2,4-oxadiazol-3-yl]-8-fluoro-1,1-dioxo-5-[[6-[4-(trifluoromethyl)phenyl]-3-pyridyl]methyl]-2,3-dihydro-1λ⁶,5-benzothiazepin-4-one;
(3R)-3-amino-8-fluoro-7-[5-(4-oxa-7-azaspiro[2.5]octan-7-yl)-1,2,4-oxadiazol-3-yl]-1,1-dioxo-5-[[6-[5-(trifluoromethyl)-1,2,4-oxadiazol-3-yl]-3-pyridyl]methyl]-2,3-dihydro-1λ⁶,5-benzothiazepin-4-one;
(3R)-3-amino-8-fluoro-1,1-dioxo-7-(5-pyrrolidin-1-yl-1,2,4-oxadiazol-3-yl)-5-[[6-[5-(trifluoromethyl)-1,2,4-oxadiazol-3-yl]-3-pyridyl]methyl]-2,3-dihydro-1λ⁶,5-benzothiazepin-4-one,
1-[3-[(3R)-3-amino-8-fluoro-1,1,4-trioxo-5-[[4-[5-(trifluoromethyl)-1,2,4-oxadiazol-3-yl]phenyl]methyl]-2,3-dihydro-6,5-benzothiazepin-7-yl]-1,2,4-oxadiazol-5-yl]cyclopropanecarbonitrile;
1-[3-[(3R)-3-amino-8-fluoro-1,1,4-trioxo-5-[[4-[5-(trifluoromethyl)-2-pyridyl]phenyl]methyl]-2,3-dihydro-1λ⁶, 5-benzothiazepin-7-yl]-1,2,4-oxadiazol-5-yl]cyclopropanecarbonitrile;
(3R)-3-amino-7-(5-tert-butyl-1,3,4-oxadiazol-2-yl)-5-[[4-(5-tert-butyl-1,3,4-oxadiazol-2-yl)phenyl]methyl]-1,1-dioxo-2,3-dihydro-1λ⁶,5-benzothiazepin-4-one;
(3R)-3-amino-7-(5-tert-butyl-1,3,4-oxadiazol-2-yl)-5-[[4-(3-methyl-1,2,4-oxadiazol-5-yl)phenyl]methyl]-1,1-dioxo-2,3-dihydro-1λ⁶,5-benzothiazepin-4-one;
(3R)-3-amino-7-(5-tert-butyl-1,3,4-oxadiazol-2-yl)-5-[[4-(1-methylpyrazol-3-yl)phenyl]methyl]-1,1-dioxo-2,3-dihydro-1λ⁶,5-benzothiazepin-4-one;
(3R)-3-amino-7-(5-tert-butyl-1,3,4-oxadiazol-2-yl)-5-[[4-(3,5-dimethylpyrazol-1-yl)phenyl]methyl]-1,1-dioxo-2,3-dihydro-1λ⁶,5-benzothiazepin-4-one;
(3R)-3-amino-7-(5-tert-butyl-1,3,4-oxadiazol-2-yl)-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-(5-tert-butyl-1,3,4-oxadiazol-2-yl)-1,1-dioxo-5-[[4-[5-(trifluoromethyl)-1,3,4-oxadiazol-2-yl]phenyl]methyl]-2,3-dihydro-1λ⁶,5-benzothiazepin-4-one;
(3R)-3-amino-7-(5-tert-butyl-1,3,4-oxadiazol-2-yl)-1,1-dioxo-5-[[4-[4-(trifluoromethyl)pyrazol-1-yl]phenyl]methyl]-2,3-dihydro-11λ⁶,5-benzothiazepin-4-one;
(3R)-3-amino-7-(5-tert-butyl-1,3,4-oxadiazol-2-yl)-1,1-dioxo-5-[[4-[3-(trifluoromethyl)-1,2,4-oxadiazol-5-yl]phenyl]methyl]-2,3-dihydro-1λ⁶,5-benzothiazepin-4-one;
(3R)-3-amino-7-(5-tert-butyl-1,3,4-oxadiazol-2-yl)-1,1-dioxo-5-[[4-[3-(trifluoromethyl)pyrazol-1-yl]phenyl]methyl]-2,3-dihydro-11λ⁶,5-benzothiazepin-4-one;
(3R)-3-amino-7-(5-tert-butyl-1,3,4-oxadiazol-2-yl)-1,1-dioxo-5-[[4-[4-(trifluoromethyl)imidazol-1-yl]phenyl]methyl]-2,3-dihydro-1λ⁶,5-benzothiazepin-4-one;
(3R)-3-amino-7-(5-tert-butyl-1,3,4-oxadiazol-2-yl)-5-[[4-(3-cyclopropyl-1,2,4-oxadiazol-5-yl)phenyl]methyl]-1,1-dioxo-2,3-dihydro-1λ⁶,5-benzothiazepin-4-one;
(3R)-3-amino-7-(5-tert-butyl-1,3,4-oxadiazol-2-yl)-1,1-dioxo-5-[[4-[5-(trifluoromethyl)isoxazol-3-yl]phenyl]methyl]-2,3-dihydro-11λ⁶,5-benzothiazepin-4-one;
(3R)-3-amino-7-(5-tert-butyl-1,3,4-oxadiazol-2-yl)-1,1-dioxo-5-[[4-[3-(trifluoromethyl)isoxazol-5-yl]phenyl]methyl]-2,3-dihydro-11λ⁶,5-benzothiazepin-4-one;
(3R)-3-amino-7-(5-tert-butyl-1,3,4-oxadiazol-2-yl)-1,1-dioxo-5-[[4-[4-(trifluoromethyl)oxazol-2-yl]phenyl]methyl]-2,3-dihydro-11λ⁶,5-benzothiazepin-4-one;
(3R)-3-amino-7-(5-tert-butyl-1,3,4-oxadiazol-2-yl)-1,1-dioxo-5-[[4-[5-(trifluoromethyl)tetrazol-2-yl]phenyl]methyl]-2,3-dihydro-11λ⁶,5-benzothiazepin-4-one;
(3R)-3-amino-7-(5-tert-butyl-1,3,4-oxadiazol-2-yl)-1,1-dioxo-5-[[4-[3-(trifluoromethyl)-1,2,4-triazol-1-yl]phenyl]methyl]-2,3-dihydro-1λ⁶,5-benzothiazepin-4-one;
(3R)-3-amino-7-(5-tert-butyl-1,3,4-oxadiazol-2-yl)-5-[[4-[2-methyl-5-(trifluoromethyl)pyrazol-3-yl]phenyl]methyl]-1,1-dioxo-2,3-dihydro-1λ⁶, 5-benzothiazepin-4-one;
(3R)-3-amino-7-(5-tert-butyl-1,3,4-oxadiazol-2-yl)-1,1-dioxo-5-[[4-[5-(trifluoromethyl)oxazol-2-yl]phenyl]methyl]-2,3-dihydro-11λ⁶,5-benzothiazepin-4-one;
(3R)-3-amino-7-[5-(1-amino-2,2,2-trifluoro-1-methyl-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one,
(3R)-3-amino-7-[5-(1-amino-2,2,2-trifluoro-1-methyl-ethyl)-1,3,4-oxadiazol-2-yl]-1,1-dioxo-5-[[4-[5-(2,2,2-trifluoroethyl)-1,2,4-oxadiazol-3-yl]phenyl]methyl]-2,3-dihydro-1λ⁶,5-benzothiazepin-4-one;
(3R)-3-amino-7-[5-(1-amino-2,2,2-trifluoro-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one;
(3R)-3-amino-7-[5-[1-hydroxy-1-(trifluoromethyl)propyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one,
(3R)-3-amino-1,1-dioxo-7-[5-(2,2,2-trifluoro-1-hydroxy-1-methyl-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-[5-(2-cyclopropyltetrahydrofuran-2-yl)-1,3,4-oxadiazol-2-yl]-1,1-dioxo-5-[[4-[5-(trifluoromethyl)-1,3,4-oxadiazol-2-yl]phenyl]methyl]-2,3-dihydro-1λ⁶,5-benzothiazepin-4-one (*);
(3R)-3-amino-7-[5-(1-amino-2,2-dimethyl-propyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one;
2-[5-[(3R)-3-amino-1,1,4-trioxo-5-[[4-[5-(trifluoromethyl)-1,2,4-oxadiazol-3-yl]phenyl]methyl]-2,3-dihydro-1λ⁶, 5-benzothiazepin-7-yl]-1,3,4-oxadiazol-2-yl]propanenitrile;
(3R)-3-amino-7-[5-(2-cyclopropyltetrahydrofuran-2-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-One;
(3R)-3-amino-1,1-dioxo-7-[5-(1,2,2,2-tetrafluoro-1-methoxy-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-One;
2-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]-2-methyl-propanenitrile;
(3R)-3-amino-7-[5-(1-aminocyclohexyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one;
2-[5-[(3R)-3-amino-5-[[4-[5-(difluoromethyl)-2-pyridyl]phenyl]methyl]-1,1,4-trioxo-2,3-dihydro-1λ⁶,5-benzothiazepin-7-yl]-1,3,4-oxadiazol-2-yl]-2-methyl-propanenitrile;
(3R)-3-amino-7-[5-(1-aminocyclohexyl)-1,3,4-oxadiazol-2-yl]-1,1-dioxo-5-[[4-[5-(trifluoromethyl)-2-pyridyl]phenyl]methyl]-2,3-dihydro-1λ⁶,5-benzothiazepin-4-one;
(3R)-3-amino-7-[5-(1-aminocyclohexyl)-1,3,4-oxadiazol-2-yl]-1,1-dioxo-5-[[4-[4-(trifluoromethyl)phenyl]phenyl]methyl]-2,3-dihydro-1)⁶,5-benzothiazepin-4-one;
(3R)-3-amino-7-[5-(1-aminocyclohexyl)-1,3,4-oxadiazol-2-yl]-5-[[4-(4-methoxyphenyl)phenyl]methyl]-1,1-dioxo-2,3-dihydro-1)⁶,5-benzothiazepin-4-one;
(3R)-3-amino-7-[5-(1-aminocyclohexyl)-1,3,4-oxadiazol-2-yl]-5-[[4-[5-(3,3-difluorocyclopentyl)-1,2,4-oxadiazol-3-yl]phenyl]methyl]-1,1-dioxo-2,3-dihydro-1λ⁶,5-benzothiazepin-4-one;
(3R)-3-amino-7-[5-(1-aminocyclohexyl)-1,3,4-oxadiazol-2-yl]-1,1-dioxo-5-[[4-[5-(2,2,2-trifluoro-1-methyl-ethyl)-1,2,4-oxadiazol-3-yl]phenyl]methyl]-2,3-dihydro-1λ⁶,5-benzothiazepin-4-one;
2-[5-[(3R)-3-amino-5-[[4-(4-methoxyphenyl)phenyl]methyl]-1,1,4-trioxo-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[5-(trifluoromethyl)-2-pyridyl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[4-(trifluoromethyl)phenyl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[2-(trifluoromethyl)pyrimidin-5-yl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-5-[[4-[5-(4,4-difluorocyclohexyl)-1,2,4-oxadiazol-3-yl]phenyl]methyl]-1,1,4-trioxo-2,3-dihydro-1λ⁶,5-benzothiazepin-7-yl]-1,3,4-oxadiazol-2-yl]-2-methyl-propanenitrile,
2-[5-[(3R)-3-amino-1,1,4-trioxo-5-[[4-[4-(trifluoromethyl)pyrazol-1-yl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-5-[[4-[5-(4,4-difluoro-1-piperidyl)-1,2,4-oxadiazol-3-yl]phenyl]methyl]-1,1,4-trioxo-2,3-dihydro-1λ⁶,5-benzothiazepin-7-yl]-1,3,4-oxadiazol-2-yl]-2-methyl-Propanenitrile;
2-[5-[(3R)-3-amino-1,1,4-trioxo-5-[[4-[5-(trifluoromethyl)oxazol-2-yl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[6-[4-(trifluoromethyl)phenyl]-3-pyridyl]methyl]-2,3-dihydro-1λ⁶,5-benzothiazepin-7-yl]-1,3,4-oxadiazol-2-yl]-2-methyl-propanenitrile (*);
2-[5-[(3R)-3-amino-5-[[4-[2-methyl-5-(trifluoromethyl)pyrazol-3-yl]phenyl]methyl]-1,1,4-trioxo-2,3-dihydro-1λ⁶,5-benzothiazepin-7-yl]-1,3,4-oxadiazol-2-yl]-2-methyl-propanenitrile;
2-[5-[(3R)-3-amino-5-[[4-[5-(difluoromethoxy)-2-pyridyl]phenyl]methyl]-1,1,4-trioxo-2,3-dihydro-1λ⁶,5-benzothiazepin-7-yl]-1,3,4-oxadiazol-2-yl]-2-methyl-propanenitrile;
2-[5-[(3R)-3-amino-5-[[4-[5-(1,1-difluoroethyl)-2-pyridyl]phenyl]methyl]-1,1,4-trioxo-2,3-dihydro-1λ⁶,5-benzothiazepin-7-yl]-1,3,4-oxadiazol-2-yl]-2-methyl-propanenitrile;
2-[5-[(3R)-3-amino-5-[[4-[4-(difluoromethoxy)-2-pyridyl]phenyl]methyl]-1,1,4-trioxo-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[2-(trifluoromethyl)-4-pyridyl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[5-(trifluoromethoxy)pyrazin-2-yl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[6-(trifluoromethyl)-3-pyridyl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-5-[[4-[6-methyl-5-(trifluoromethyl)-2-pyridyl]phenyl]methyl]-1,1,4-trioxo-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[1-(trifluoromethyl)pyrazol-4-yl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[4-(trifluoromethyl)-2-pyridyl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[5-(trifluoromethoxy)pyrimidin-2-yl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-5-[[4-(5-cyclopropyl-2-pyridyl)phenyl]methyl]-1,1,4-trioxo-2,3-dihydro-1λ⁶, 5-benzothiazepin-7-yl]-1,3,4-oxadiazol-2-yl]-2-methyl-propanenitrile;
2-[5-[(3R)-3-amino-1,1,4-trioxo-5-[[6-[4-(trifluoromethoxy)phenyl]-3-pyridyl]methyl]-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[5-(trifluoromethyl)tetrazol-2-yl]phenyl]methyl]-2,3-dihydro-120,5-benzothiazepin-7-yl]-1,3,4-oxadiazol-2-yl]-2-methyl-propanenitrile;
2-[5-[(3R)-3-amino-1,1,4-trioxo-5-[[4-[3-(trifluoromethyl)pyrazol-1-yl]phenyl]methyl]-2,3-dihydro-120,5-benzothiazepin-7-yl]-1,3,4-oxadiazol-2-yl]-2-methyl-propanenitrile;
2-[5-[(3R)-3-amino-1,1,4-trioxo-5-[[4-[4-(trifluoromethyl)oxazol-2-yl]phenyl]methyl]-2,3-dihydro-120,5-benzothiazepin-7-yl]-1,3,4-oxadiazol-2-yl]-2-methyl-propanenitrile;
2-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-5-[[4-(5-methoxy-2-pyridyl)phenyl]methyl]-1,1,4-trioxo-2,3-dihydro-1λ⁶, 5-benzothiazepin-7-yl]-1,3,4-oxadiazol-2-yl]-2-methyl-propanenitrile;
2-[5-[(3R)-3-amino-1,1,4-trioxo-5-[[4-[4-(trifluoromethoxy)-2-pyridyl]phenyl]methyl]-2,3-dihydro-120,5-benzothiazepin-7-yl]-1,3,4-oxadiazol-2-yl]-2-methyl-propanenitrile;
2-[5-[(3R)-3-amino-1,1,4-trioxo-5-[[4-[5-(trifluoromethyl)pyrimidin-2-yl]phenyl]methyl]-2,3-dihydro-120,5-benzothiazepin-7-yl]-1,3,4-oxadiazol-2-yl]-2-methyl-propanenitrile;
2-[5-[(3R)-3-amino-1,1,4-trioxo-5-[[4-[5-(1,1,2,2,2-pentafluoroethoxy)-2-pyridyl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[5-(2,2,2-trifluoroethoxy)-2-pyridyl]phenyl]methyl]-2,3-dihydro-120,5-benzothiazepin-7-yl]-1,3,4-oxadiazol-2-yl]-2-methyl-propanenitrile;
2-[5-[(3R)-3-amino-1,1,4-trioxo-5-[[4-[6-(trifluoromethoxy)-3-pyridyl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[4-(trifluoromethoxy)pyrazol-1-yl]phenyl]methyl]-2,3-dihydro-120,5-benzothiazepin-7-yl]-1,3,4-oxadiazol-2-yl]-2-methyl-propanenitrile;
2-[5-[(3R)-3-amino-5-[[4-(3-cyclopropyl-1,2,4-oxadiazol-5-yl)phenyl]methyl]-1,1,4-trioxo-2,3-dihydro-1λ⁶,5-benzothiazepin-7-yl]-1,3,4-oxadiazol-2-yl]-2-methyl-propanenitrile;
2-[5-[(3R)-3-amino-5-[[4-[4-methyl-5-(trifluoromethoxy)-2-pyridyl]phenyl]methyl]-1,1,4-trioxo-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[4-(trifluoromethyl)triazol-1-yl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-5-[[4-[2-methyl-5-(trifluoromethoxy)pyrazol-3-yl]phenyl]methyl]-1,1,4-trioxo-2,3-dihydro-1λ⁶,5-benzothiazepin-7-yl]-1,3,4-oxadiazol-2-yl]-2-methyl-propanenitrile;
(3R)-3-amino-7-[5-(2-methyloxetan-2-yl)-1,3,4-oxadiazol-2-yl]-1,1-dioxo-5-[[4-[4-(trifluoromethyl)phenyl]phenyl]methyl]-2,3-dihydro-1lambda6,5-benzothiazepin-4-one;
(3R)-3-amino-7-[5-(3-methyloxetan-3-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one;
(3R)-3-amino-7-[5-(3-methyloxetan-3-yl)-1,3,4-oxadiazol-2-yl]-1,1-dioxo-5-[[4-[5-(trifluoromethyl)-2-pyridyl]phenyl]methyl]-2,3-dihydro-1λ⁶,5-benzothiazepin-4-one;
(3R)-3-amino-7-[5-(1-amino-2,2,2-trifluoro-ethyl)-1,3,4-oxadiazol-2-yl]-1,1-dioxo-5-[[4-[4-(trifluoromethyl)phenyl]phenyl]methyl]-2,3-dihydro-1)⁶,5-benzothiazepin-4-one;
(3R)-3-amino-7-[5-[3-(difluoromethyl)azetidin-3-yl]-1,3,4-oxadiazol-2-yl]-1,1-dioxo-5-[[4-[4-(trifluoromethyl)phenyl]phenyl]methyl]-2,3-dihydro-1)⁶,5-benzothiazepin-4-one;
(3R)-3-amino-7-[5-(3-fluoro-1-methyl-3-piperidyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one;
(3R)-3-amino-7-[5-(3-fluoro-1-methyl-3-piperidyl)-1,3,4-oxadiazol-2-yl]-5-[[4-(4-methoxyphenyl)phenyl]methyl]-1,1-dioxo-2,3-dihydro-1)⁶,5-benzothiazepin-4-one;
(3R)-3-amino-7-[5-(3-fluoro-1-methyl-3-piperidyl)-1,3,4-oxadiazol-2-yl]-1,1-dioxo-5-[[4-[5-(trifluoromethyl)-2-pyridyl]phenyl]methyl]-2,3-dihydro-1λ⁶,5-benzothiazepin-4-one;
(3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-[5-[(1S)-1-amino-2,2,2-trifluoro-1-methyl-ethyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethoxy)-2-pyridyl]phenyl]methyl]-2,3-dihydro-1λ⁶,5-benzothiazepin-4-one;
(3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethyl)-2-pyridyl]phenyl]methyl]-2,3-dihydro-1λ⁶,5-benzothiazepin-4-one;
(3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethyl)tetrazol-2-yl]phenyl]methyl]-2,3-dihydro-1λ⁶,5-benzothiazepin-4-one;
(3R)-3-amino-7-[5-[(1R)-1-amino-2,2,2-trifluoro-1-methyl-ethyl]-1,3,4-oxadiazol-2-yl]-1,1-dioxo-5-[[4-[3-(trifluoromethyl)pyrazol-1-yl]phenyl]methyl]-2,3-dihydro-1λ⁶, 5-benzothiazepin-4-one;
(3R)-3-amino-7-[5-[2-(hydroxymethyl)tetrahydrofuran-2-yl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-[5-[2-(hydroxymethyl)tetrahydrofuran-2-yl]-1,3,4-oxadiazol-2-yl]-1,1-dioxo-5-[[4-[4-(trifluoromethyl)phenyl]phenyl]methyl]-2,3-dihydro-1λ⁶, 5-benzothiazepin-4-one;
methyl 3,3-difluoro-5-[5-[(3R)-3-amino-5-[[4-(5-tert-butyl-1,2,4-oxadiazol-3-yl)phenyl]methyl]-1,1,4-trioxo-2,3-dihydro-1λ⁶,5-benzothiazepin-7-yl]-1,3,4-oxadiazol-2-yl]piperidine-1-carboxylate;
methyl 3,3-difluoro-5-[5-[(3R)-3-amino-1,1,4-trioxo-5-[[6-[4-(trifluoromethyl)phenyl]-3-pyridyl]methyl]-2,3-dihydro-1λ⁶,5-benzothiazepin-7-yl]-1,3,4-oxadiazol-2-yl]piperidine-1-carboxylate;
methyl 1-[5-[(3R)-3-amino-1,1,4-trioxo-5-[[6-[4-(trifluoromethyl)phenyl]-3-pyridyl]methyl]-2,3-dihydro-1λ⁶,5-benzothiazepin-7-yl]-1,3,4-oxadiazol-2-yl]-3-azabicyclo[3.1.1]heptane-3-carboxylate;
methyl 1-[5-[(3R)-3-amino-5-[[4-(4-methoxyphenyl)phenyl]methyl]-1,1,4-trioxo-2,3-dihydro-1λ⁶,5-benzothiazepin-7-yl]-1,3,4-oxadiazol-2-yl]-3-azabicyclo[3.1.1]heptane-3-carboxylate;
methyl 1-[5-[(3R)-3-amino-1,1,4-trioxo-5-[[4-[4-(trifluoromethyl)phenyl]phenyl]methyl]-2,3-dihydro-1λ⁶,5-benzothiazepin-7-yl]-1,3,4-oxadiazol-2-yl]-3-azabicyclo[3.1.1]heptane-3-carboxylate;
methyl 1-[5-[(3R)-3-amino-1,1,4-trioxo-5-[[4-[5-(trifluoromethyl)-1,2,4-oxadiazol-3-yl]phenyl]methyl]-2,3-dihydro-1λ⁶, 5-benzothiazepin-7-yl]-1,3,4-oxadiazol-2-yl]-3-azabicyclo[3.1.1]heptane-3-carboxylate;
1-[5-[(3R)-3-amino-1,1,4-trioxo-5-[[4-[5-(trifluoromethyl)-2-pyridyl]phenyl]methyl]-2,3-dihydro-1λ⁶,5-benzothiazepin-7-yl]-1,3,4-oxadiazol-2-yl]cyclopropanecarbonitrile;
methyl 1-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]-3-azabicyclo[3.1.1]heptane-3-carboxylate;
methyl 1-[5-[(3R)-3-amino-1,1,4-trioxo-5-[[4-[5-(trifluoromethyl)-2-pyridyl]phenyl]methyl]-2,3-dihydro-1λ⁶,5-benzothiazepin-7-yl]-1,3,4-oxadiazol-2-yl]-3-azabicyclo[3.1.1]heptane-3-carboxylate;
(3R)-3-amino-7-[5-[2-(hydroxymethyl)tetrahydrofuran-2-yl]-1,3,4-oxadiazol-2-yl]-5-[[4-(4-methoxyphenyl)phenyl]methyl]-1,1-dioxo-2,3-dihydro-1)⁶,5-benzothiazepin-4-one;
(3R)-3-amino-7-[5-(1-ethoxy-1,2,2,2-tetrafluoro-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one;
((3R)-3-amino-5-[[4-(5-cyclopropyl-1,3,4-oxadiazol-2-yl)phenyl]methyl]-1,1-dioxo-7-[5-(1,2,2,2-tetrafluoro-1-methoxy-ethyl)-1,3,4-oxadiazol-2-yl]-2,3-dihydro-1λ⁶,5-benzothiazepin-4-One;
(3R)-3-amino-5-[[4-(1-cyclopropyl-1,2,4-triazol-3-yl)phenyl]methyl]-1,1-dioxo-7-[5-(1,2,2,2-tetrafluoro-1-methoxy-ethyl)-1,3,4-oxadiazol-2-yl]-2,3-dihydro-1λ⁶,5-benzothiazepin-4-one;
(3R)-3-amino-1,1-dioxo-7-[5-(1,2,2,2-tetrafluoro-1-methoxy-ethyl)-1,3,4-oxadiazol-2-yl]-5-[[4-[5-(trifluoromethyl)-1,3,4-oxadiazol-2-yl]phenyl]methyl]-2,3-dihydro-1λ⁶,5-benzothiazepin-4-One;
(3R)-3-amino-7-[5-(4,4-difluoro-1-piperidyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one;
(3R)-3-amino-5-[[4-(5-cyclopropyl-1,2,4-oxadiazol-3-yl)phenyl]methyl]-7-[5-(4,4-difluoro-1-piperidyl)-1,3,4-oxadiazol-2-yl]-1,1-dioxo-2,3-dihydro-1λ⁶,5-benzothiazepin-4-one;
(3R)-3-amino-5-[[4-[5-(difluoromethyl)-1,3,4-oxadiazol-2-yl]phenyl]methyl]-7-[5-(4,4-difluoro-1-piperidyl)-1,3,4-oxadiazol-2-yl]-1,1-dioxo-2,3-dihydro-1λ⁶,5-benzothiazepin-4-one;
(3R)-3-amino-7-[5-(4,4-difluoro-1-piperidyl)-1,3,4-oxadiazol-2-yl]-1,1-dioxo-5-[[4-[5-(trifluoromethyl)-1,3,4-oxadiazol-2-yl]phenyl]methyl]-2,3-dihydro-1λ⁶, 5-benzothiazepin-4-one;
(3R)-3-amino-7-[5-(4,4-difluoro-1-piperidyl)-1,3,4-oxadiazol-2-yl]-1,1-dioxo-5-[[4-[5-(trifluoromethyl)-2-pyridyl]phenyl]methyl]-2,3-dihydro-1λ⁶,5-benzothiazepin-4-one;
(3R)-3-amino-7-[5-(4,4-difluoro-1-piperidyl)-1,3,4-oxadiazol-2-yl]-1,1-dioxo-5-[[4-[3-(trifluoromethyl)-1,2,4-oxadiazol-5-yl]phenyl]methyl]-2,3-dihydro-1λ⁶, 5-benzothiazepin-4-one;
(3R)-3-amino-7-[5-(4,4-difluoro-1-piperidyl)-1,3,4-oxadiazol-2-yl]-1,1-dioxo-5-[[4-[3-(trifluoromethyl)pyrazol-1-yl]phenyl]methyl]-2,3-dihydro-1λ⁶,5-benzothiazepin-4-one;
4-[5-[(3R)-3-amino-1,1,4-trioxo-5-[[4-[5-(trifluoromethyl)-1,2,4-oxadiazol-3-yl]phenyl]methyl]-2,3-dihydro-1λ⁶, 5-benzothiazepin-7-yl]-1,3,4-oxadiazol-2-yl]morpholine-2-carbonitrile;
(3R)-3-amino-7-[5-[2-(methoxymethyl)morpholin-4-yl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-One;
(3R)-3-amino-7-[5-[(3,3-difluoro-1-methyl-cyclobutyl)amino]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-1,1-dioxo-7-[5-[2-(trifluoromethyl)morpholin-4-yl]-1,3,4-oxadiazol-2-yl]-5-[[4-[4-(trifluoromethyl)phenyl]phenyl]methyl]-2,3-dihydro-1λ⁶,5-benzothiazepin-4-one;
(3R)-3-amino-1,1-dioxo-7-[5-[2-(trifluoromethyl)morpholin-4-yl]-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-One;
(3R)-3-amino-1,1-dioxo-7-[5-[2-(trifluoromethyl)morpholin-4-yl]-1,3,4-oxadiazol-2-yl]-5-[[4-[5-(trifluoromethyl)-2-pyridyl]phenyl]methyl]-2,3-dihydro-1λ⁶,5-benzothiazepin-4-one;
(3R)-3-amino-7-[5-(3,3-difluoropyrrolidin-1-yl)-1,3,4-oxadiazol-2-yl]-5-[[4-(4-methoxyphenyl)phenyl]methyl]-1,1-dioxo-2,3-dihydro-1λ⁶,5-benzothiazepin-4-one;
(3R)-3-amino-7-[5-(4-oxa-7-azaspiro[2.5]octan-7-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one;
(3R)-3-amino-7-[5-(2,2-difluoromorpholin-4-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶, 5-benzothiazepin-4-one;
(3R)-3-amino-7-[5-(2,2-difluoromorpholin-4-yl)-1,3,4-oxadiazol-2-yl]-5-[[4-(4-methoxyphenyl)phenyl]methyl]-1,1-dioxo-2,3-dihydro-1λ⁶,5-benzothiazepin-4-one;
(3R)-3-amino-7-[5-(2,2-difluoromorpholin-4-yl)-1,3,4-oxadiazol-2-yl]-1,1-dioxo-5-[[4-[5-(trifluoromethyl)-1,3,4-oxadiazol-2-yl]phenyl]methyl]-2,3-dihydro-1λ⁶, 5-benzothiazepin-4-one;
(3R)-3-amino-5-[[4-(4-methoxyphenyl)phenyl]methyl]-7-[5-[methyl(2,2,2-trifluoroethyl)amino]-1,3,4-oxadiazol-2-yl]-1,1-dioxo-2,3-dihydro-1λ⁶, 5-benzothiazepin-4-one;
(3R)-3-amino-5-[[4-[5-(difluoromethyl)-1,3,4-oxadiazol-2-yl]phenyl]methyl]-7-[5-(2,2-difluoromorpholin-4-yl)-1,3,4-oxadiazol-2-yl]-1,1-dioxo-2,3-dihydro-1λ⁶, 5-benzothiazepin-4-one;
(3R)-3-amino-7-(5-tert-butyl-1,3,4-oxadiazol-2-yl)-5-[[4-(6-methoxy-3-pyridyl)phenyl]methyl]-1,1-dioxo-2,3-dihydro-11λ⁶,5-benzothiazepin-4-one;
(3R)-3-amino-1,1-dioxo-7-[5-(2,2,2-trifluoro-1,1-dimethyl-ethyl)-1,3,4-oxadiazol-2-yl]-5-[[4-[5-(trifluoromethoxy)-2-pyridyl]phenyl]methyl]-2,3-dihydro-1λ⁶,5-benzothiazepin-4-one;
(3R)-3-amino-1,1-dioxo-7-[5-(2,2,2-trifluoro-1,1-dimethyl-ethyl)-1,3,4-oxadiazol-2-yl]-5-[[4-[5-(trifluoromethyl)-1,2,4-oxadiazol-3-yl]phenyl]methyl]-2,3-dihydro-1λ⁶, 5-benzothiazepin-4-one;
(3R)-3-amino-5-[[4-(5-cyclopropyl-1,2,4-oxadiazol-3-yl)phenyl]methyl]-1,1-dioxo-7-[5-(2,2,2-trifluoro-1,1-dimethyl-ethyl)-1,3,4-oxadiazol-2-yl]-2,3-dihydro-1λ⁶,5-benzothiazepin-4-one;
(3R)-3-amino-5-[[4-(5-cyclopropyl-1,2,4-oxadiazol-3-yl)phenyl]methyl]-1,1-dioxo-7-[5-(1,2,2,2-tetrafluoroethyl)-1,3,4-oxadiazol-2-yl]-2,3-dihydro-1λ⁶,5-benzothiazepin-4-one;
(3R)-3-amino-5-[[4-(5-cyclopropyl-1,2,4-oxadiazol-3-yl)phenyl]methyl]-7-[5-[1-hydroxy-1-(trifluoromethyl)propyl]-1,3,4-oxadiazol-2-yl]-1,1-dioxo-2,3-dihydro-1λ⁶,5-benzothiazepin-4-One;
(3R)-3-amino-5-[[4-(5-cyclopropyl-1,2,4-oxadiazol-3-yl)phenyl]methyl]-1,1-dioxo-7-[5-(2,2,2-trifluoro-1-hydroxy-1-methyl-ethyl)-1,3,4-oxadiazol-2-yl]-2,3-dihydro-1λ⁶,5-benzothiazepin-4-one;
(3R)-3-amino-7-[5-(1-amino-1-cyclopropyl-2,2,2-trifluoro-ethyl)-1,3,4-oxadiazol-2-yl]-5-[[4-(5-cyclopropyl-1,2,4-oxadiazol-3-yl)phenyl]methyl]-1,1-dioxo-2,3-dihydro-1λ⁶,5-benzothiazepin-4-one;
1-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]cyclobutanecarbonitrile;
4-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]tetrahydropyran-4-carbonitrile;
3-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-2,2-dimethyl-propanenitrile;
3-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-3-methyl-butanenitrile;
2-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-2-ethyl-butanenitrile;
3-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]tetrahydrofuran-3-carbonitrile;
(3R)-3-amino-7-[5-(1-amino-4,4-difluoro-cyclohexyl)-1,3,4-oxadiazol-2-yl]-1,1-dioxo-5-[[4-[5-(trifluoromethoxy)-2-pyridyl]phenyl]methyl]-2,3-dihydro-1λ⁶,5-benzothiazepin-4-one;
(3R)-3-amino-7-[5-(1-amino-3,3-difluoro-cyclobutyl)-1,3,4-oxadiazol-2-yl]-1,1-dioxo-5-[[4-[5-(trifluoromethoxy)-2-pyridyl]phenyl]methyl]-2,3-dihydro-1λ⁶,5-benzothiazepin-4-one;
4-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-1-methyl-piperidine-4-carbonitrile;
(3R)-3-amino-5-[[4-(5-tert-butyl-1,2,4-oxadiazol-3-yl)phenyl]methyl]-7-[5-(1-ethyl-5,5-difluoro-3-piperidyl)-1,3,4-oxadiazol-2-yl]-1,1-dioxo-2,3-dihydro-1λ⁶,5-benzothiazepin-4-one;
(3R)-3-amino-7-[5-[5,5-difluoro-1-(2-methoxyethyl)-3-piperidyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-[5-[5,5-difluoro-1-(2-methoxyethyl)-3-piperidyl]-1,3,4-oxadiazol-2-yl]-5-[[4-(4-methoxyphenyl)phenyl]methyl]-1,1-dioxo-2,3-dihydro-1)⁶,5-benzothiazepin-4-one;
(3R)-3-amino-7-[5-(3-aminooxetan-3-yl)-1,2,4-oxadiazol-3-yl]-1,1-dioxo-5-[[4-[5-(trifluoromethyl)-1,2,4-oxadiazol-3-yl]phenyl]methyl]-2,3-dihydro-1lambda6,5-benzothiazepin-4-one;
(3R)-3-amino-7-[5-(2-chloro-3-pyridyl)-1,2,4-oxadiazol-3-yl]-5-[[4-(5-cyclopropyl-1,2,4-oxadiazol-3-yl)phenyl]methyl]-1,1-dioxo-2,3-dihydro-1λ⁶,5-benzothiazepin-4-one;
(3R)-3-amino-5-[[4-(5-cyclopropyl-1,2,4-oxadiazol-3-yl)phenyl]methyl]-7-[5-(6-fluoro-2-methyl-3-pyridyl)-1,2,4-oxadiazol-3-yl]-1,1-dioxo-2,3-dihydro-1λ⁶,5-benzothiazepin-4-one; and
(3R)-3-amino-5-[[4-(5-cyclopropyl-1,2,4-oxadiazol-3-yl)phenyl]methyl]-1,1-dioxo-7-[5-[2-(trifluoromethyl)-3-pyridyl]-1,2,4-oxadiazol-3-yl]-2,3-dihydro-1λ⁶,5-benzothiazepin-4-one; or a pharmaceutically acceptable salt thereof.

13. The compound of claim 1, or a pharmaceutically acceptable salt thereof, selected from:
(3R)-3-amino-8-fluoro-7-[5-(4-oxa-7-azaspiro[2.5]octan-7-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-[5-(4,4-difluoro-1-piperidyl)-1,3,4-oxadiazol-2-yl]-8-fluoro-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-[5-(3-aminooxetan-3-yl)-1,2,4-oxadiazol-3-yl]-8-fluoro-1,1-dioxo-5-[[4-[5-(trifluoromethyl)-1,2,4-oxadiazol-3-yl]phenyl]methyl]-2,3-dihydro-1λ⁶,5-benzothiazepin-4-One;
1-[3-[(3R)-3-amino-8-fluoro-1,1,4-trioxo-5-[[4-[5-(trifluoromethyl)-1,2,4-oxadiazol-3-yl]phenyl]methyl]-2,3-dihydro-6,5-benzothiazepin-7-yl]-1,2,4-oxadiazol-5-yl]cyclopropanecarbonitrile;
1-[3-[(3R)-3-amino-8-fluoro-1,1,4-trioxo-5-[[4-[5-(trifluoromethyl)-2-pyridyl]phenyl]methyl]-2,3-dihydro-6,5-benzothiazepin-7-yl]-1,2,4-oxadiazol-5-yl]cyclopropanecarbonitrile;
(3R)-3-amino-8-fluoro-7-[5-[2-(hydroxymethyl)tetrahydrofuran-2-yl]-1,2,4-oxadiazol-3-yl]-1,1-dioxo-5-[[4-[5-(trifluoromethyl)-1,2,4-oxadiazol-3-yl]phenyl]methyl]-2,3-dihydro-16,5-benzothiazepin-4-one;
(3R)-3-amino-7-(5-tert-butyl-1,3,4-oxadiazol-2-yl)-1,1-dioxo-5-[[4-[5-(trifluoromethyl)-1,3,4-oxadiazol-2-yl]phenyl]methyl]-2,3-dihydro-1λ⁶, 5-benzothiazepin-4-one;
(3R)-3-amino-7-(5-tert-butyl-1,3,4-oxadiazol-2-yl)-1,1-dioxo-5-[[4-[3-(trifluoromethyl)-1,2,4-oxadiazol-5-yl]phenyl]methyl]-2,3-dihydro-1λ⁶,5-benzothiazepin-4-one;
(3R)-3-amino-7-(5-tert-butyl-1,3,4-oxadiazol-2-yl)-5-[[4-(3-cyclopropyl-1,2,4-oxadiazol-5-yl)phenyl]methyl]-1,1-dioxo-2,3-dihydro-11λ⁶,5-benzothiazepin-4-one;
(3R)-3-amino-7-[5-(1-amino-2,2,2-trifluoro-1-methyl-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
2-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]propanenitrile;
(3R)-3-amino-7-[5-(2-cyclopropyltetrahydrofuran-2-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-1,1-dioxo-7-[5-(1,2,2,2-tetrafluoro-1-methoxy-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one;
2-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]-2-methyl-propanenitrile;
(3R)-3-amino-7-[5-(1-aminocyclohexyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
2-[5-[(3R)-3-amino-5-[[4-(4-methoxyphenyl)phenyl]methyl]-1,1,4-trioxo-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[4-(trifluoromethyl)phenyl]phenyl]methyl]-2,3-dihydro-1λ⁶,5-benzothiazepin-7-yl]-1,3,4-oxadiazol-2-yl]-2-methyl-propanenitrile;
(3R)-3-amino-7-[5-[(1R)-1-amino-2,2,2-trifluoro-1-methyl-ethyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-[5-[(1S)-1-amino-2,2,2-trifluoro-1-methyl-ethyl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-[5-[2-(hydroxymethyl)tetrahydrofuran-2-yl]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
methyl 1-[5-[(3R)-3-amino-5-[[4-(4-methoxyphenyl)phenyl]methyl]-1,1,4-trioxo-2,3-dihydro-1λ⁶,5-benzothiazepin-7-yl]-1,3,4-oxadiazol-2-yl]-3-azabicyclo[3.1.1]heptane-3-carboxylate;
methyl 1-[5-[(3R)-3-amino-1,1,4-trioxo-5-[[4-[4-(trifluoromethyl)phenyl]phenyl]methyl]-2,3-dihydro-1λ⁶,5-benzothiazepin-7-yl]-1,3,4-oxadiazol-2-yl]-3-azabicyclo[3.1.1]heptane-3-Carboxylate;
methyl 1-[5-[(3R)-3-amino-1,1,4-trioxo-5-[[4-[5-(trifluoromethyl)-1,2,4-oxadiazol-3-yl]phenyl]methyl]-2,3-dihydro-1λ⁶,5-benzothiazepin-7-yl]-1,3,4-oxadiazol-2-yl]-3-azabicyclo[3.1.1]heptane-3-carboxylate;
(3R)-3-amino-7-[5-(1-ethoxy-1,2,2,2-tetrafluoro-ethyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-[5-(4,4-difluoro-1-piperidyl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-One;
(3R)-3-amino-7-[5-[(3,3-difluoro-1-methyl-cyclobutyl)amino]-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-1,1-dioxo-7-[5-[2-(trifluoromethyl)morpholin-4-yl]-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-[5-(2,2-difluoromorpholin-4-yl)-1,3,4-oxadiazol-2-yl]-1,1-dioxo-5-[[4-[5-(trifluoromethyl)-1,2,4-oxadiazol-3-yl]phenyl]methyl]-2,3-dihydro-1λ⁶,5-benzothiazepin-4-one;
(3R)-3-amino-7-[5-(2,2-difluoromorpholin-4-yl)-1,3,4-oxadiazol-2-yl]-1,1-dioxo-5-[[4-[5-(trifluoromethyl)-1,3,4-oxadiazol-2-yl]phenyl]methyl]-2,3-dihydro-1λ⁶,5-benzothiazepin-4-one;
(3R)-3-amino-5-[[4-(4-methoxyphenyl)phenyl]methyl]-7-[5-[methyl(2,2,2-trifluoroethyl)amino]-1,3,4-oxadiazol-2-yl]-1,1-dioxo-2,3-dihydro-1λ⁶,5-benzothiazepin-4-one;
(3R)-3-amino-1,1-dioxo-7-[5-(2,2,2-trifluoro-1,1-dimethyl-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one;
2-[5-[(3R)-3-amino-1,1,4-trioxo-5-[[4-[5-(trifluoromethoxy)-2-pyridyl]phenyl]methyl]-2,3-dihydro-1λ, 5-benzothiazepin-7-yl]-1,3,4-oxadiazol-2-yl]-2-methyl-propanenitrile;
(3R)-3-amino-1,1-dioxo-7-[5-(2,2,2-trifluoro-1-hydroxy-1-methyl-ethyl)-1,3,4-oxadiazol-2-yl]-5-[[4-[5-(trifluoromethyl)-1,2,4-oxadiazol-3-yl]phenyl]methyl]-2,3-dihydro-1λ⁶,5-benzothiazepin-4-one;
2-[5-[(3R)-3-amino-8-fluoro-1,1,4-trioxo-5-[[4-[5-(trifluoromethyl)-2-pyridyl]phenyl]methyl]-2,3-dihydro-6,5-benzothiazepin-7-yl]-1,3,4-oxadiazol-2-yl]-2-methyl-propanenitrile;
(3R)-3-amino-1,1-dioxo-7-[5-(2,2,2-trifluoro-1,1-dimethyl-ethyl)-1,3,4-oxadiazol-2-yl]-5-[[4-[5-(trifluoromethoxy)-2-pyridyl]phenyl]methyl]-2,3-dihydro-1λ⁶,5-benzothiazepin-4-one;
2-[5-[(3R)-3-amino-1,1,4-trioxo-5-[[4-[5-(trifluoromethoxy)pyrimidin-2-yl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[5-(1,1,2,2,2-pentafluoroethoxy)-2-pyridyl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[4-(trifluoromethoxy)pyrazol-1-yl]phenyl]methyl]-2,3-dihydro-1λ⁶,5-benzothiazepin-7-yl]-1,3,4-oxadiazol-2-yl]-2-methyl-propanenitrile;
2-[5-[(3R)-3-amino-1,1,4-trioxo-5-[[4-[5-(trifluoromethoxy)-2-pyridyl]phenyl]methyl]-2,3-dihydro-1λ⁶,5-benzothiazepin-7-yl]-1,3,4-oxadiazol-2-yl]-2-ethyl-butanenitrile; and
2-[5-[(3R)-3-amino-5-[[4-[2-methyl-5-(trifluoromethoxy)pyrazol-3-yl]phenyl]methyl]-1,1,4-trioxo-2,3-dihydro-k, 5-benzothiazepin-7-yl]-1,3,4-oxadiazol-2-yl]-2-methyl-propanenitrile, or a pharmaceutically acceptable salt thereof.

14. A process for the preparation of a compound of formula (I) 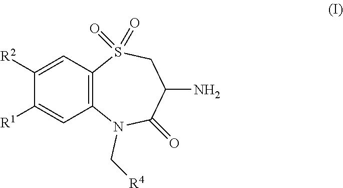
or a pharmaceutically acceptable salt thereof, wherein:
R¹ is oxadiazole, wherein R¹ is optionally substituted with one or more R¹⁰ which can be the same or different;
R² is selected from hydrogen and halogen;
R⁴ is selected from C₅₋₁₄-aryl and 5-14 membered heteroaryl, wherein R⁴ is optionally substituted with one or more R¹¹ which can be the same or different;
R¹⁰ is selected from:
vi) C₁₋₁₀-alkyl, optionally substituted with one or more halogen, amino, hydroxy, C₁₋₆-alkoxy, 3-10 membered cycloalkyl, phenyl, cyano;
vii) C₃₋₁₀-cycloalkyl, optionally substituted with one or more halogen, cyano, amino;
viii) 3-10 membered heterocyclyl, optionally substituted with one or more halogen, C₁₋₁₀-alkyl, amino, halo-C₁₋₆-alkyl, hydroxy, cyano, —C(O)O—(R^{10q}), C₃₋₁₀-cycloalkyl, wherein C₁₋₁₀-alkyl is optionally substituted with one or more hydroxy, C₁₋₆-alkoxy;
ix) —N(R^{10e}R^{10f});
x) heteroaryl, optionally substituted with one or more C₁₋₁₀-alkyl, halogen;
R^{10e} and R^{10f} are each independently selected from:
iv) hydrogen;
v) C₁₋₆-alkyl, optionally substituted with one or more, cyano, halogen, hydroxy;
vi) C₃₋₁₀-cycloalkyl, optionally substituted with one or more halogen, C₁₋₁₀-alkyl;
R^{10q} is C₁₋₅-alkyl, wherein C₁₋₅-alkyl is optionally substituted with one or more hydroxy;
R¹¹ is selected from:
vi) 5-6 membered heteroaryl, optionally substituted with one or more C₁₋₆-alkyl, C₃₋₁₀ cycloalkyl, halo-C₁₋₆-alkyl, C₁₋₆-alkoxy, halo-C₁₋₆-alkoxy, wherein C₃₋₁₀ cycloalkyl is optionally substituted with one or more halogen;
phenyl, optionally substituted with one or more C₁₋₆-alkoxy, —OH, halo-C₁₋₆-alkyl, comprising reacting a compound of formula (IX) 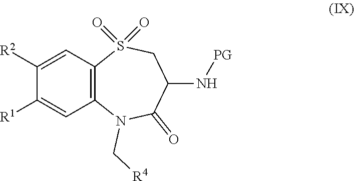
wherein R¹, R², and R⁴ are as defined in formula (I) and PG is an amino protecting group, with a suitable deprotection agent to form said compound of formula (I).

15-16. (canceled)

17. A pharmaceutical composition comprising a compound according to claim 1, or a pharmaceutically acceptable salt thereof, and a pharmaceutically acceptable excipient.

18. The pharmaceutical composition according to claim 17, further comprising an additional therapeutic agent.

19-23. (canceled)

24. A method for the treatment and/or delay of progression of cancer in a subject in need thereof, comprising administering to the subject a therapeutically effective amount of a compound according to claim 1, or a pharmaceutically acceptable salt thereof, wherein the cancer is associated with aberrant diacylglycerol kinase signaling, wherein the diacylglycerol kinase is selected from DGKα and/or DGKζ.

25. (canceled)

26. A method of inhibiting activity of at least one of diacylglycerol kinases selected from DGKα and DGKζ in a subject in need thereof, comprising administering to the subject in need thereof a therapeutically effective amount of a compound according to claim 1, or a pharmaceutically acceptable salt thereof.

27. (canceled)

28. The method of claim 24, wherein the cancer is selected from the group consisting of B-cell acute lymphoid leukemia, T-cell acute lymphoid leukemia, acute lymphoid leukemia, chronic myelogenous leukemia, chronic lymphocytic leukemia B-cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom's macroglobulinemia, preleukemia, sarcoma, carcinoma, melanoma, neuroblastoma, renal cell carcinoma, colon cancer, colorectal cancer, breast cancer, epithelial squamous cell cancer, melanoma, stomach cancer, brain cancer, lung cancer, pancreatic cancer, cervical cancer, ovarian cancer, liver cancer, bladder cancer, prostate cancer, testicular cancer, thyroid cancer, uterine cancer, adrenal cancer, and head and neck cancer.
